(19) United States
(12) Patent Application Publication
(10) Pub. No.: US 2024/0166641 A1
(43) Pub. Date: May 23, 2024

(54) HETEROCYCLES AND USES THEREOF

(71) Applicant: Kumquat Biosciences Inc., San Diego, CA (US)

(72) Inventors: Liansheng Li, San Diego, CA (US); Xiuwen Zhu, San Diego, CA (US); Pingda Ren, San Diego, CA (US); Jonathan Deane, San Diego, CA (US); Lomon So, Poway, CA (US); Yi Liu, San Diego, CA (US)

(21) Appl. No.: 18/463,134

(22) Filed: Sep. 7, 2023

Related U.S. Application Data
(63) Continuation of application No. PCT/US2022/019840, filed on Mar. 10, 2022.
(60) Provisional application No. 63/159,966, filed on Mar. 11, 2021.

(51) Int. Cl. C07D 417/14; A61P 35/00; C07D 417/04; C07D 471/04

(52) U.S. Cl. CPC C07D 417/14; A61P 35/00; C07D 417/04; C07D 471/04

(57) ABSTRACT

The present disclosure provides compounds and pharmaceutically acceptable salt thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of International Application No. PCT/US2022/019840, filed Mar. 10, 2022, which claims the benefit of U.S. Provisional Patent Application 63/159,966, filed on Mar. 11, 2021, each of which is incorporated herein by reference in its entirety.

### SEQUENCE LISTING

The instant application contains a Sequence Listing which has been submitted electronically in XML format and is hereby incorporated by references in its entirety. Said XML copy, created Sep. 7, 2023, is named 56690_729_301_SL.xml and is 4,760 bytes in size.

### BACKGROUND

A number of protein tyrosine phosphatase have been implicated in regulating a diversity of cellular processes and known to be involved a range of diseases. Amongst them are metabolic, cardiovascular, neurological and cancerous disease conditions. PTPN2 encodes a protein tyrosine phosphatase that has been implicated in a number of intracellular signaling pathways of immune cells. PTPN2 can negatively regulate as TCR T cell receptor (TCR) signaling by dephosphorylating and inactivating, e.g., the Src family kinase including LCK. In addition, PTPN2 can antagonize growth factor or cytokine-mediated signaling required for T cell function, homeostasis, and/or differentiation by dephosphorylating and inactivating JAK family kinases, e.g., JAK-1 and JAK-3, and/or target substrates of the JAK family kinases, e.g., STAT-1, STAT-3, and STAT-5.

Based on genome-wide association studies, PTPN2 single nucleotide polymorphisms (SNPs) have been linked with the development of several human autoimmune diseases including, but are not limited to, type 1 diabetes, rheumatoid arthritis, Crohn's disease, and celiac disease. For example, a PTPN2 variant, rs1893217(C), has been associated with about 40% decrease in PTPN2 mRNA expression in CD4+ T cells, as well as the development of type 1 diabetes. In addition, PTPN2 mRNA expression levels in lung cancer tissues have been shown to be higher than those in normal lung tissues or adjacent normal tissues, such overexpression of PTPN2 promoting proliferation of lung cancer cells. Furthermore, two PTPN2 SNPs, rs2847297 and rs2847282, have been associated with a decrease in both PTPN2 mRNA expression and lung cancer risk, especially squamous cell lung carcinoma risk.

Cancer is the second leading cause of human death. There were close to 10 million deaths from cancer worldwide in 2018 and 17 million new cases were diagnosed. In the United States alone, cancer causes the death of over a half-million people annually, with some 1.7 million new cases diagnosed per year (excluding basal cell and squamous cell skin cancers). Lung, liver, stomach, and bowel are the most common causes of cancer death worldwide, accounting for more than four in ten of all cancer deaths.

### SUMMARY OF THE INVENTION

In view of the foregoing, there exists a considerable need for alternative compositions and methods to carry out treatment for diseases associated with PTPN2 activity. The compositions and methods of the present disclosure address these needs and provide additional advantages.

In an aspect is provided a compound of Formula (I-3), or a pharmaceutically acceptable salt or solvate thereof:

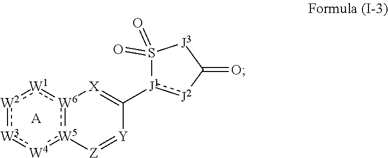

wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R^{2a}), C(O), S(O), S(O)₂, S(O)(NR²), S(O)(R²), or P(O)(R)
W³ is N, N(R³), C(R³), C(R³)(R^{3a}), C(O), S(O), S(O)₂, S(O)(NR³), S(O)(R³), or P(O)(R³)
W⁴ is N, N(R⁴), C(R⁴), C(R⁴)(R^{4a}), C(O), S(O), S(O)₂, S(O)(NR⁴), S(O)(R⁴), or P(O)(R⁴).
W⁵ is N or C;
W⁶ is N or C; wherein at least one of W¹, W², W³, W⁴, W⁵, and W⁶ is N, N(R¹), N(R²), N(R³), or N(R⁴); and
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰);
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and provided that:
at least two of Ring A [symbol] are double bonds.

In some embodiments, X is C(R⁵).

In some embodiments, Y is C(R⁶).

In some embodiments, Z is C(R⁷).

In some embodiments, J¹ is N and J² is C(R⁹)(R^{9a}).

In some embodiments, J¹ is C(R⁸) and J² is C(R⁹)(R^{9a}).

In some embodiments, J² is CH₂.

In some embodiments, J¹ is C and J² is C(R⁹).

The compound of claim 8, or a pharmaceutically acceptable salt or solvate thereof, wherein J² is C(H).

In some embodiments, J³ is N(H).

In some embodiments, the subject compound, or a pharmaceutically acceptable salt or solvate thereof, has the structure of Formula (Ia-3), (Ib-3), or (Ic-3):

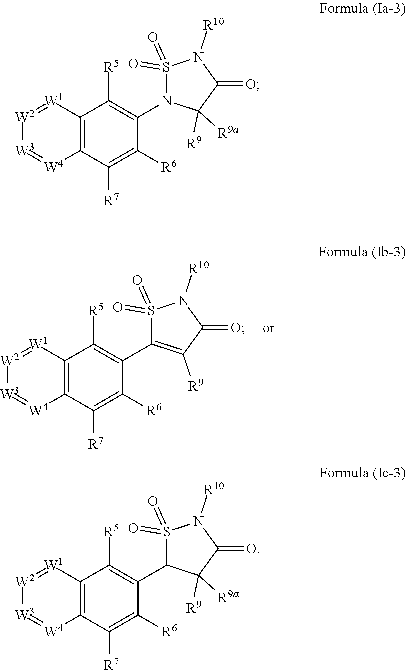

In some embodiments, the subject compound, or a pharmaceutically acceptable salt or solvate thereof, has the structure of Formula (Ia-3):

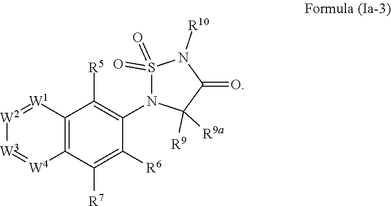

In some embodiments, V¹ is N, W² is C(R²), W³ is C(R³), and W⁴ is C(R⁴).

In some embodiments, R² is hydrogen, R³ is hydrogen, and R⁴ is hydrogen.

In some embodiments, the subject compound, or a pharmaceutically acceptable salt or solvate thereof, has the structure of Formula (Ig-3), (Ih-3), or (Ii-3):

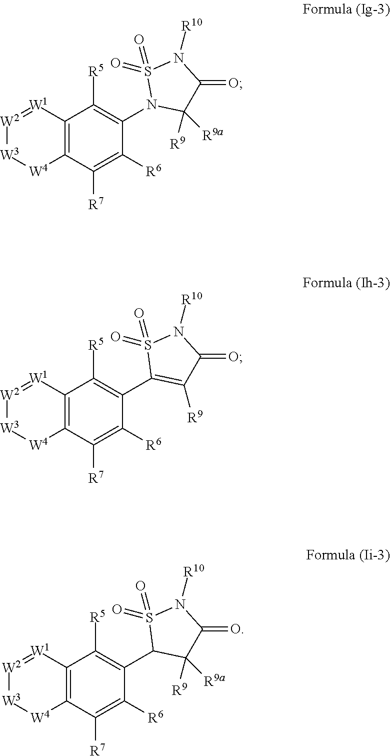

In some embodiments, the subject compound, or a pharmaceutically acceptable salt or solvate thereof, has the structure of Formula (Ig-3):

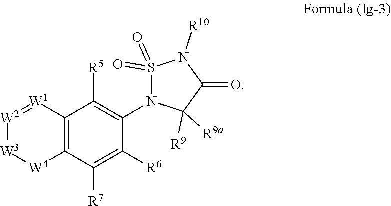

In some embodiments, W¹ is C(R¹) and W² is C(R²).

In some embodiments, R¹ is hydrogen and R² is hydrogen.

In some embodiments, W³ is C(O) and W⁴ is N(R⁴).

In some embodiments, R⁴ is methyl.

In an aspect is provided a acompound of Formula (III-3), or a pharmaceutically acceptable salt or solvate thereof:

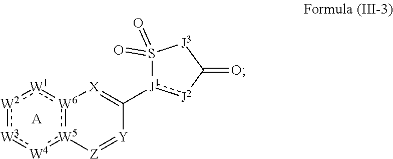

wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R^{2a}), C(O), S(O), S(O)₂, S(O)(NR²), S(O)(R²), or P(O)(R²)
W³ is N, N(R³), C(R³), C(R³)(R^{3a}), C(O), S(O), S(O)₂, S(O)(NR³), S(O)(R³), or P(O)(R³)
W⁴ is N, N(R⁴), C(R⁴), C(R⁴)(R^{4a}), C(O), S(O), S(O)₂, S(O)(NR⁴), S(O)(R⁴), or P(O)(R⁴).
W⁵ is N or C;
W⁶ is N or C; wherein
at least one of W¹, W², W³, W⁴, W⁵, and W⁶ is N, N(R¹), N(R²), N(R³), N(R⁴), or O; and
at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied.

In an aspect is provided a compound of Formula (II-3), or a pharmaceutically acceptable salt or solvate thereof:

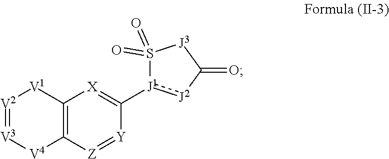

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³)
X is N or C(R⁵);
Y is N or C(R⁶
Z is N or C(R⁷)
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl.

In some embodiments, R⁵ is selected from hydrogen and halogen.

In some embodiments, R⁶ is —OH.

In some embodiments, R⁷ is hydrogen.

In some embodiments, R⁹ is hydrogen, R^{9a} is hydrogen, and R¹⁰ is hydrogen.

In some embodiments, R² is —OR¹²; and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}.

In some embodiments, R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

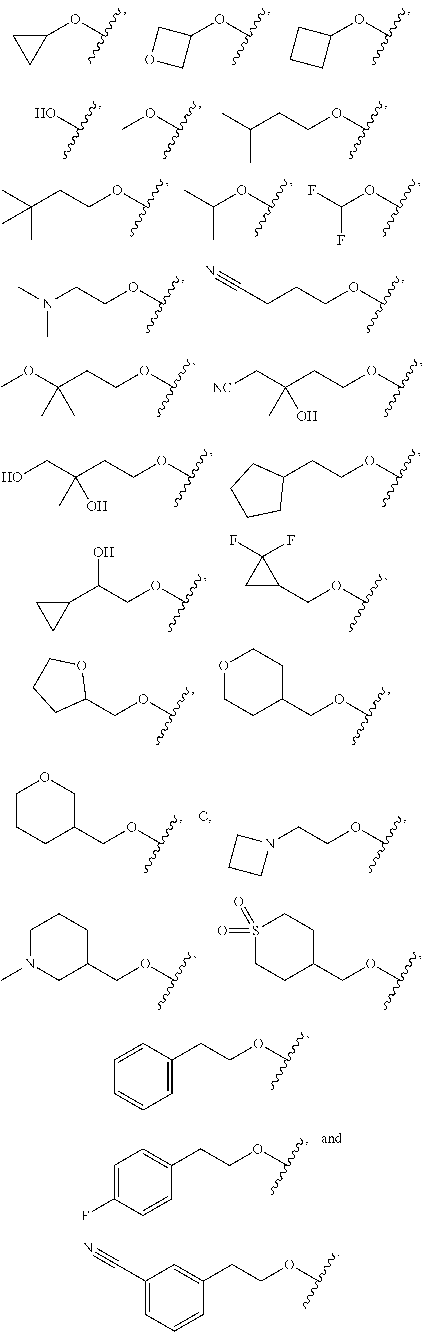

In some embodiments, R² is —N(R¹²)(R¹³); R¹³ is independently hydrogen; and R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three R^{20k}.

In some embodiments, R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

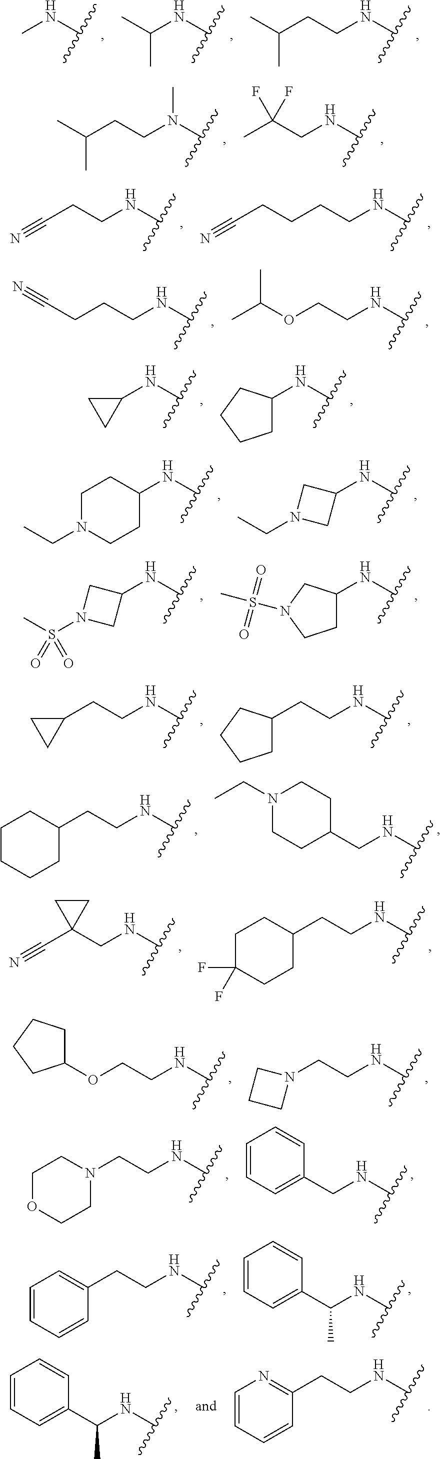

In some embodiments, R² is selected from C₁₋₆alkyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkynyl, C₃₋₆cycloalkcyl, C₂₋₉heterocycloalkcyl, C₆₋₁₀aryl, and C₁₋₉heteroaryl are optionally substituted with one, two, or three R^{20b}.

In some embodiments, R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkcyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from is

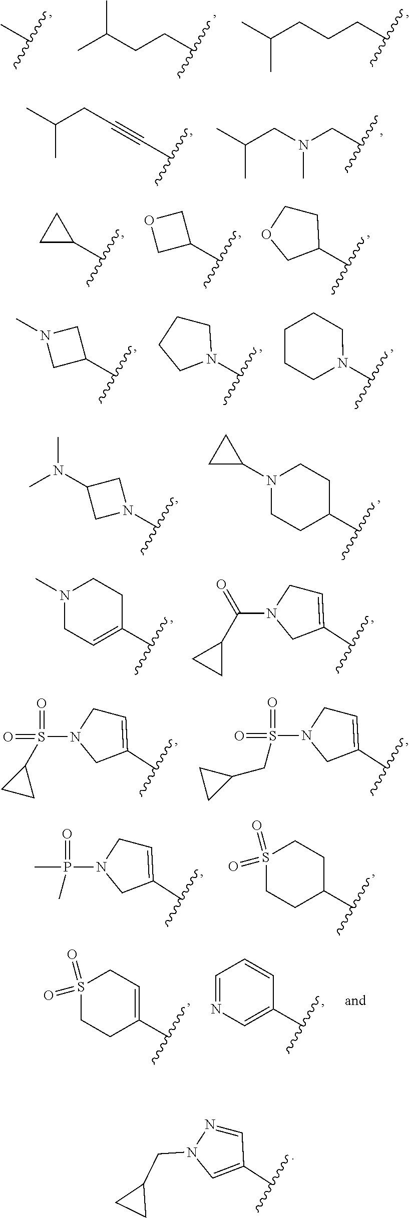

In some embodiments, R² is selected from C₁₋₆alkyl, C₂₋₉heterocycloalkyl, C₁₋₉heteroaryl, —OR¹², —N(R¹²)(R¹³), —C(O)R¹⁵, —C(O)N(R¹²)(R¹³), wherein C₁₋₆alkyl, C₂₋₉heterocycloalkyl, and C₁₋₉heteroaryl are optionally substituted with one, two, or three R^{20b}.

In some embodiments, R² is selected from —OR¹², —N(R¹²)(R¹³), —C(O)R¹⁵, —C(O)N(R¹²)(R¹³);
R¹³ is hydrogen;
R¹² and R¹⁵ are independently

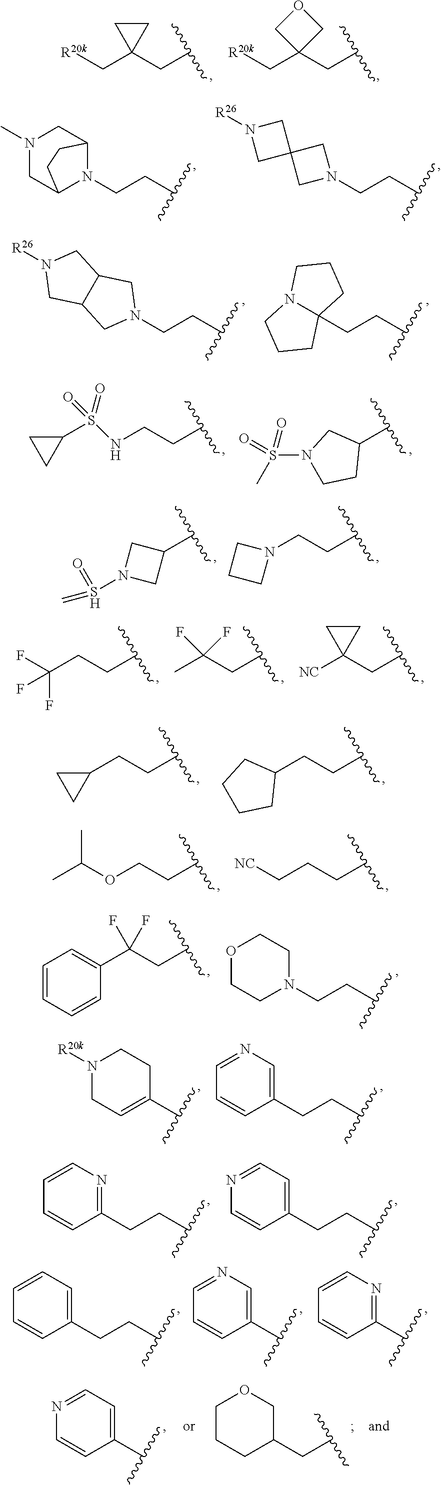

R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from —OR¹², —N(R¹²)(R¹³), —C(O)R¹⁵, —C(O)N(R¹²)(R¹³);
R¹³ is hydrogen;
R¹² and R¹⁵ are independently

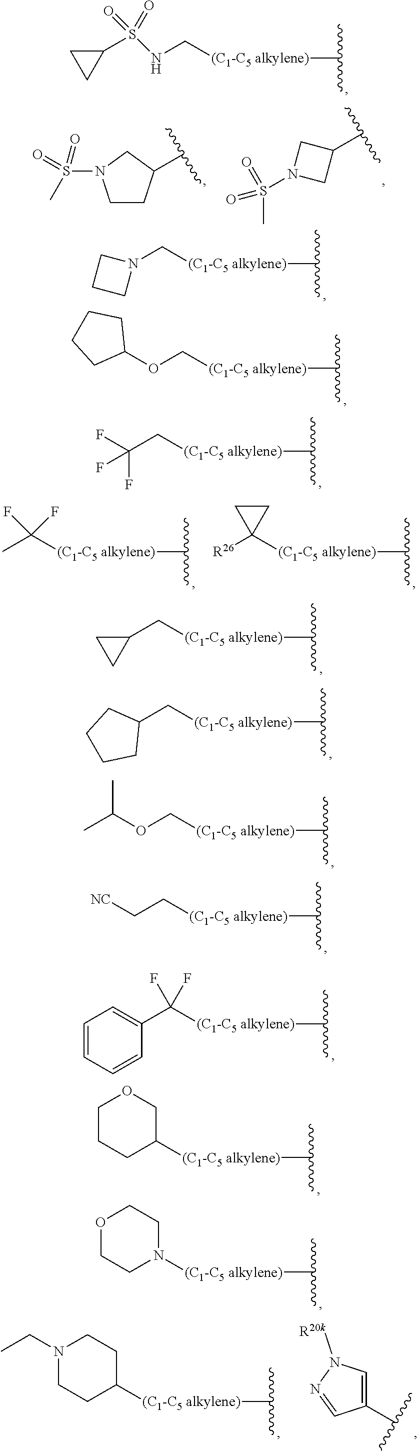

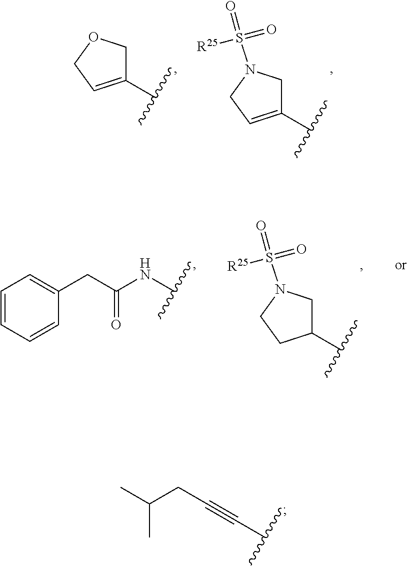

and

R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

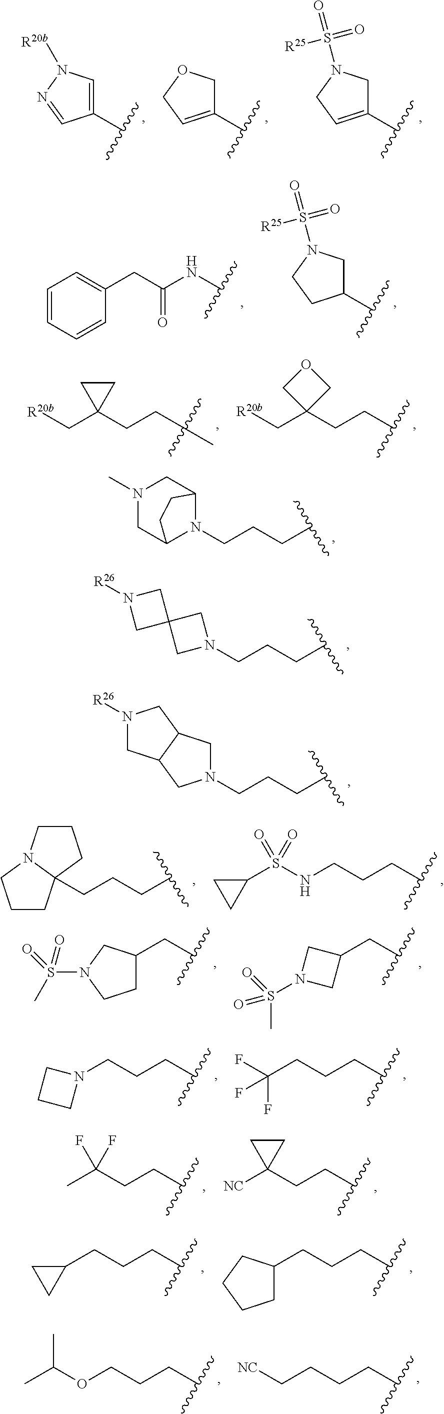

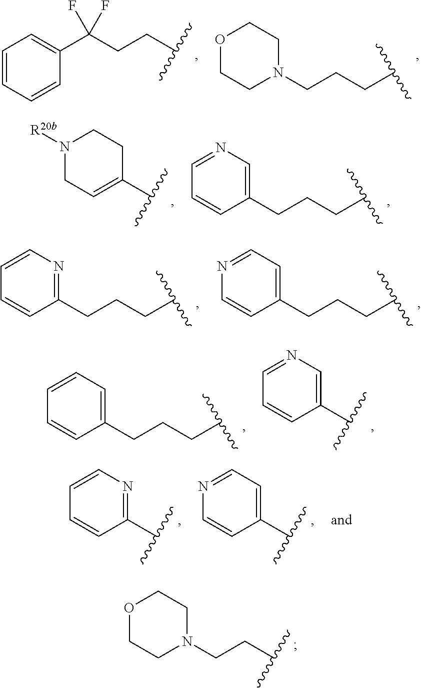

and

R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. n

In some embodiments, R² is independently

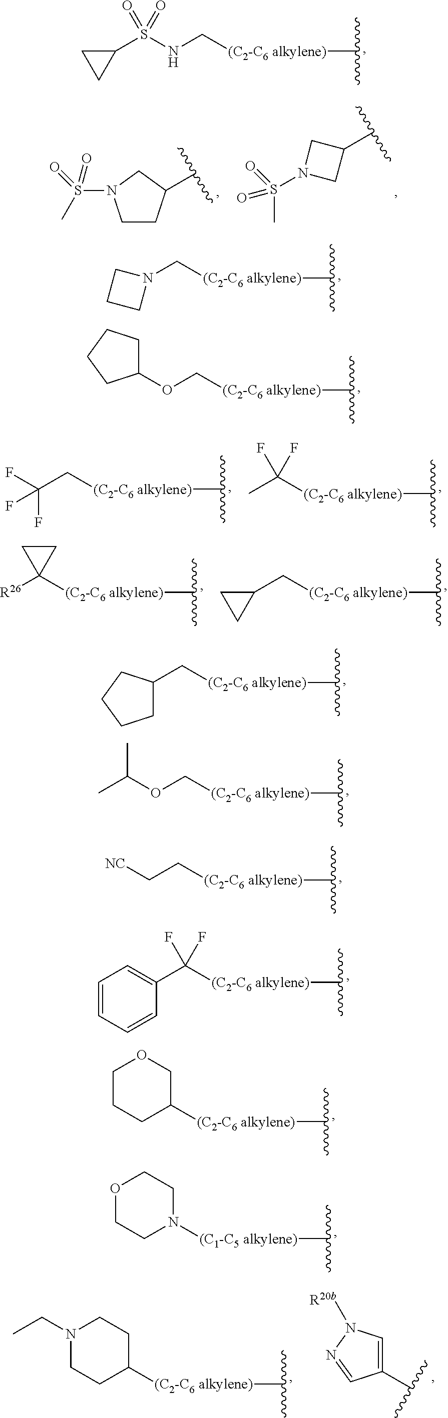

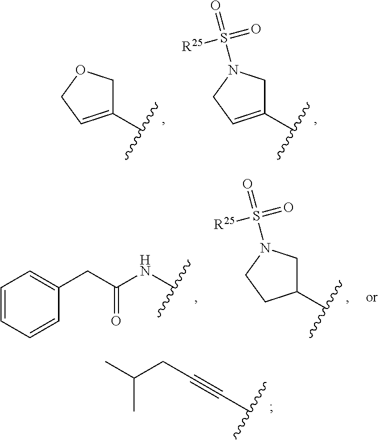

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}.

In some embodiments, R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}.

In an aspect is provided a compound, or a pharmaceutically acceptable salt or solvate thereof, selected from:

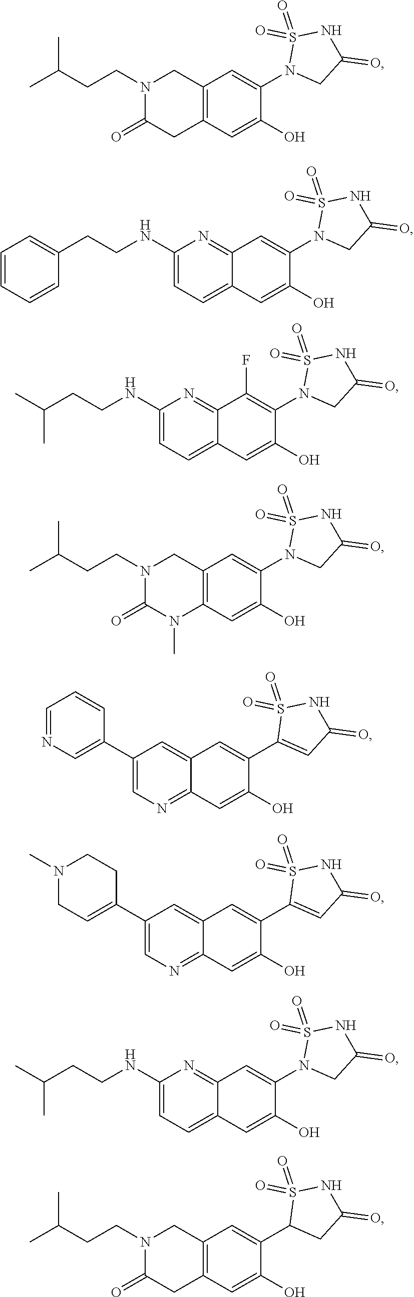

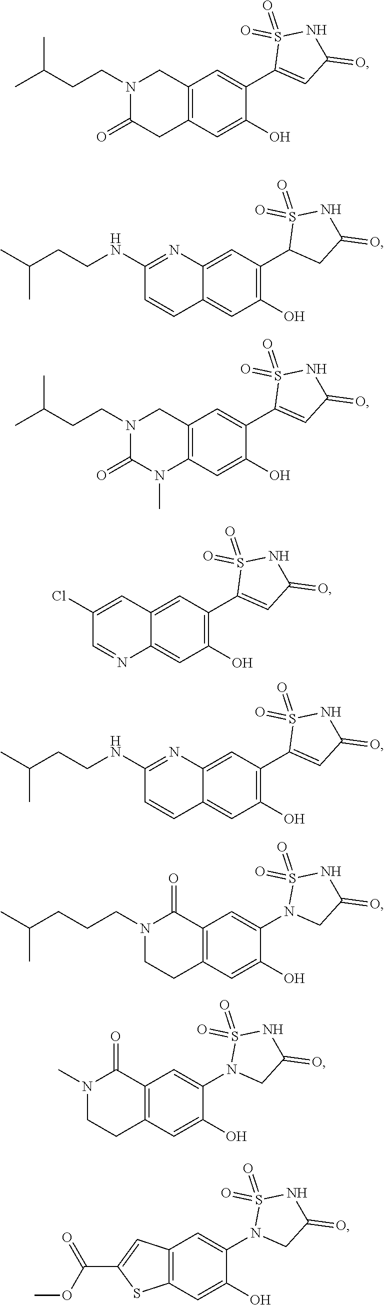

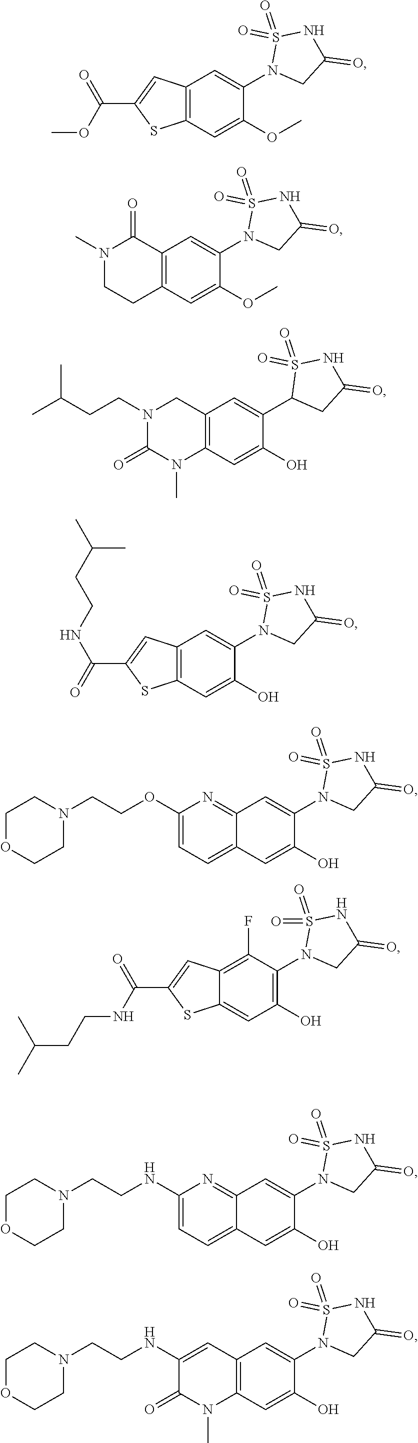

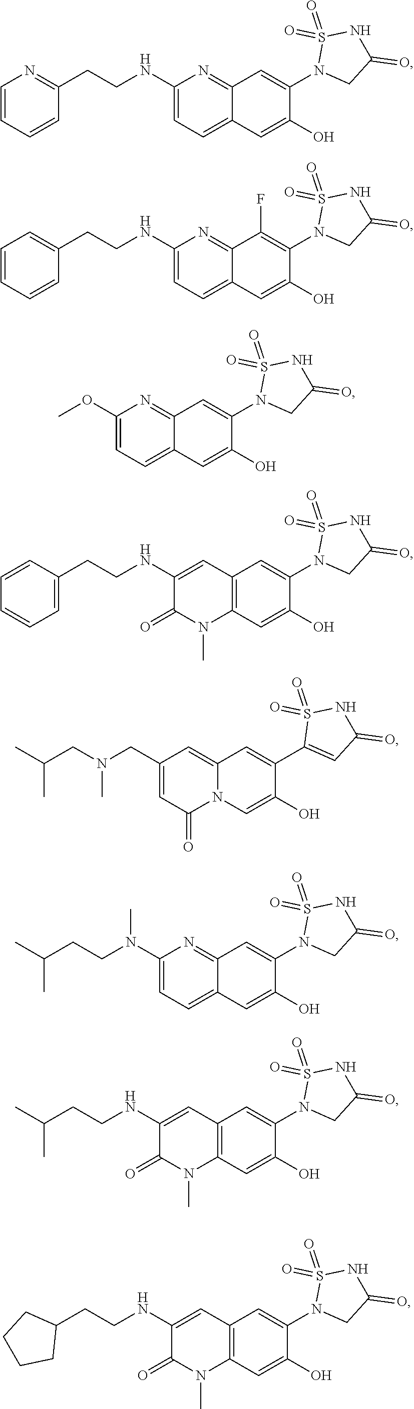

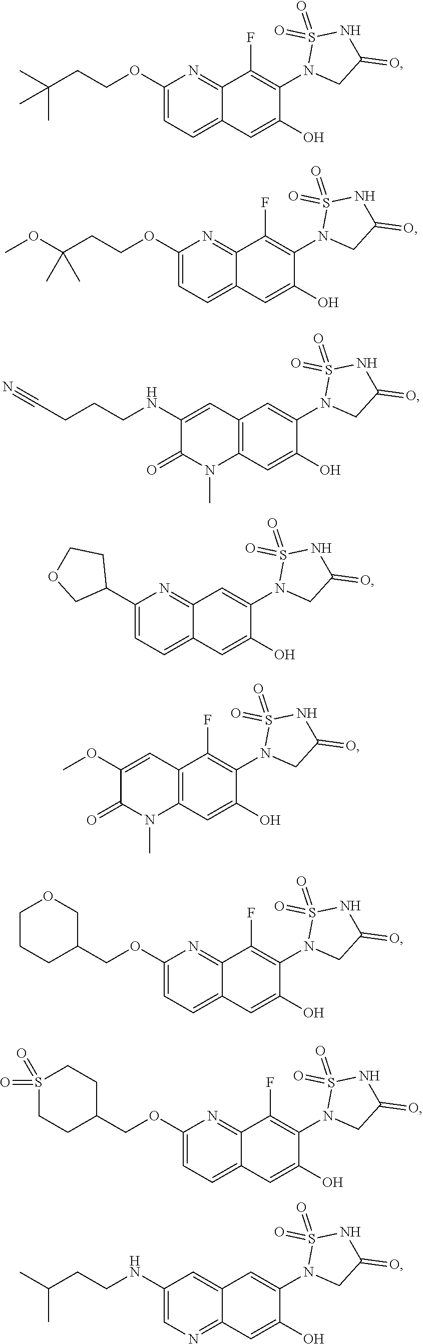

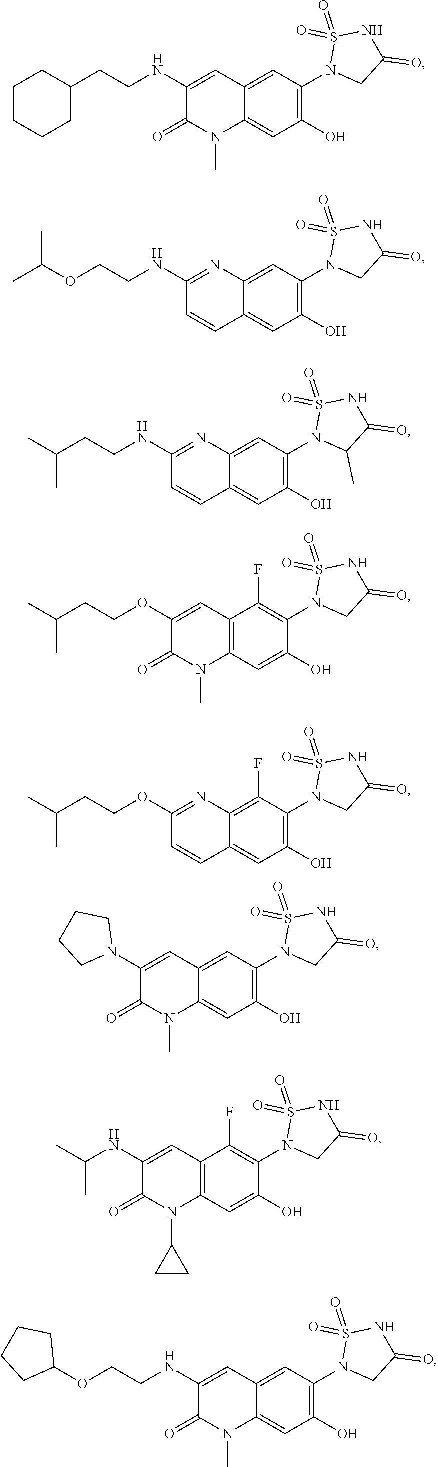

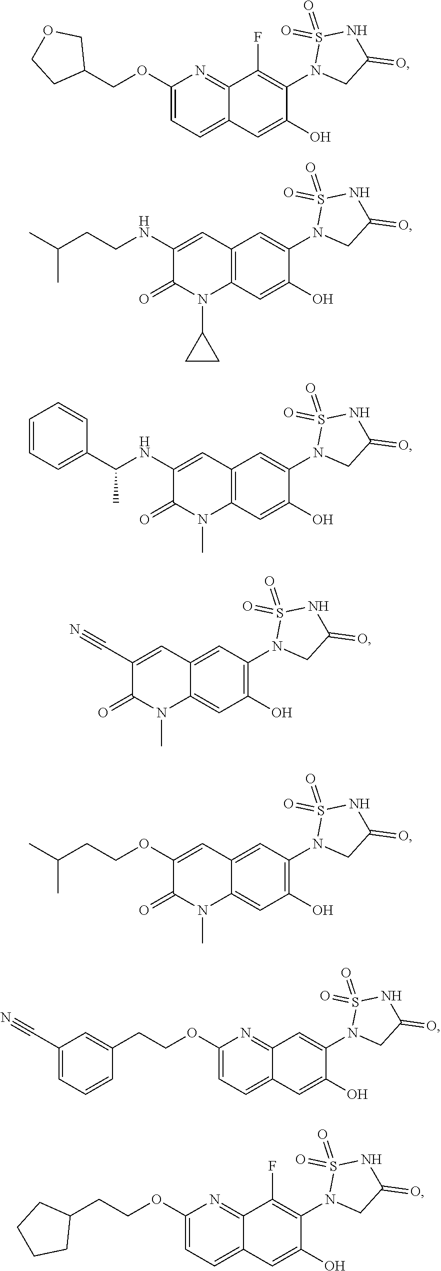

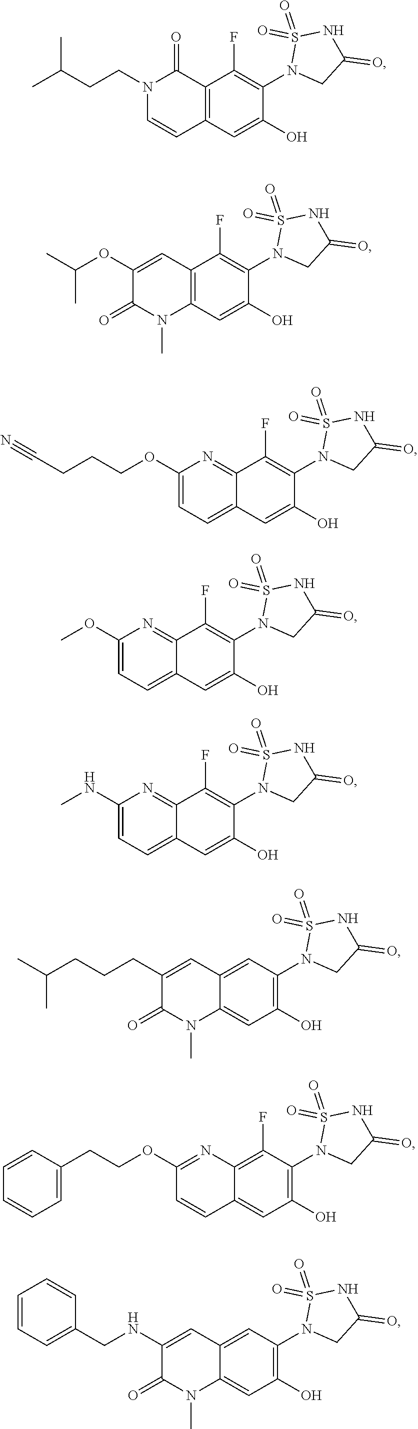

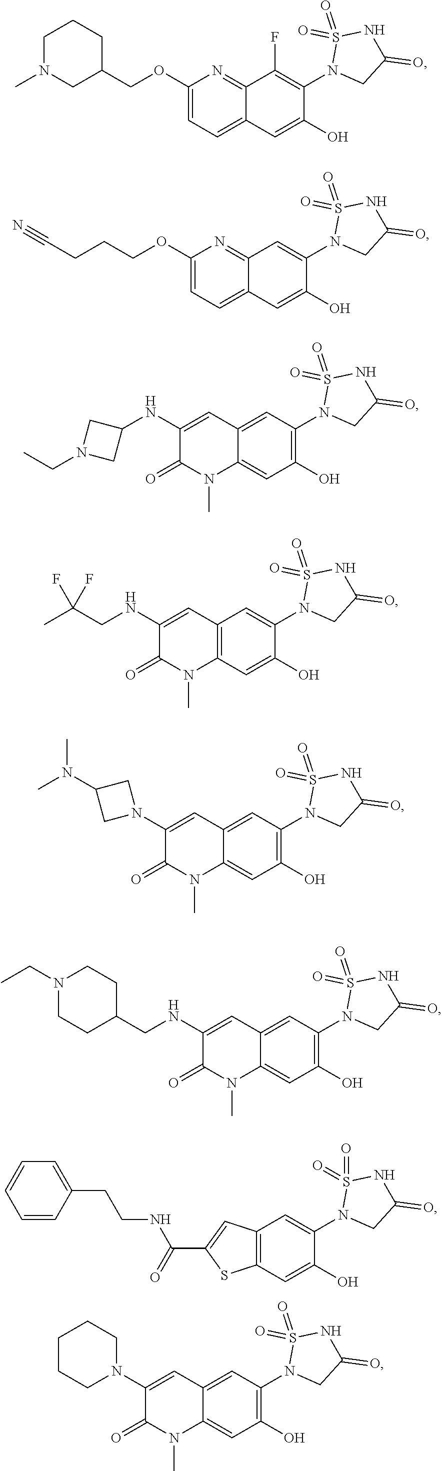

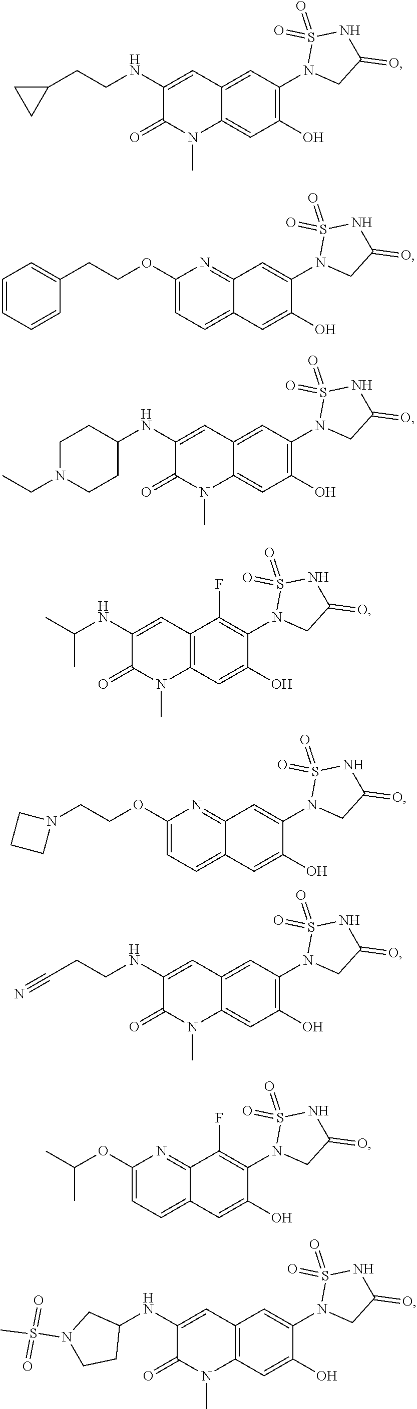

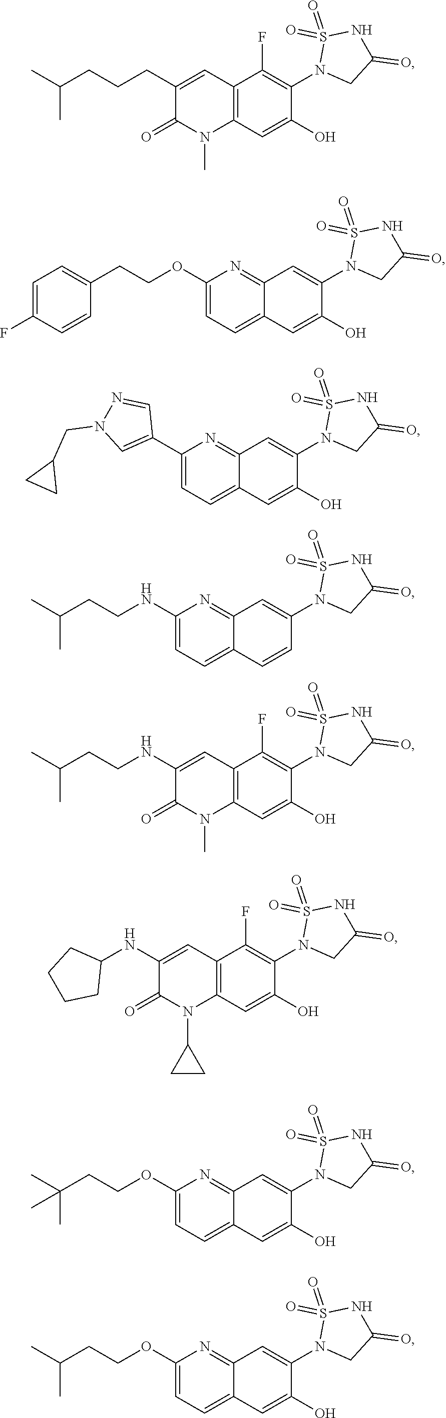

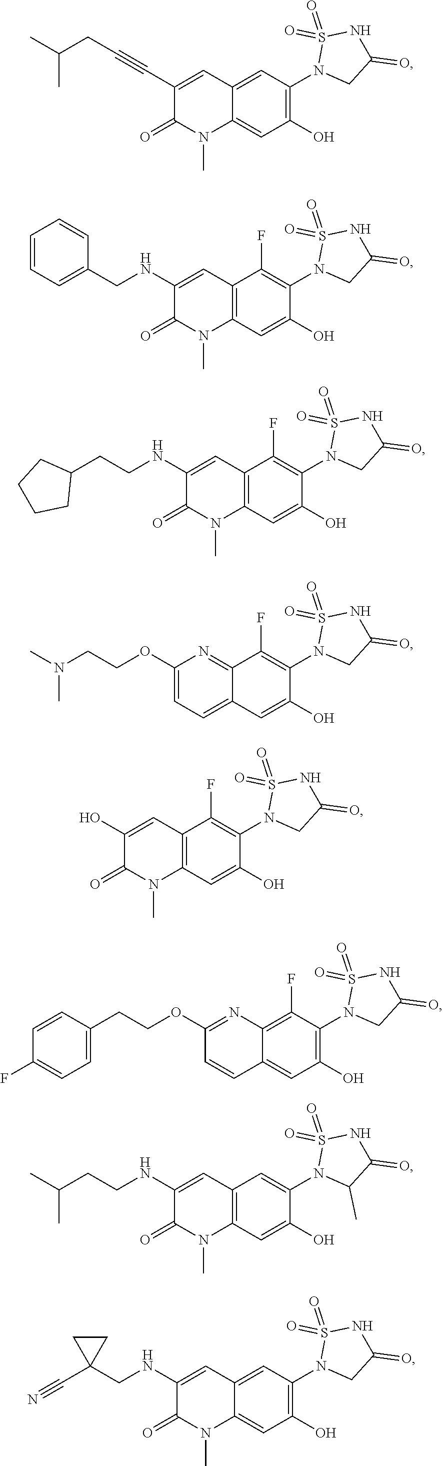

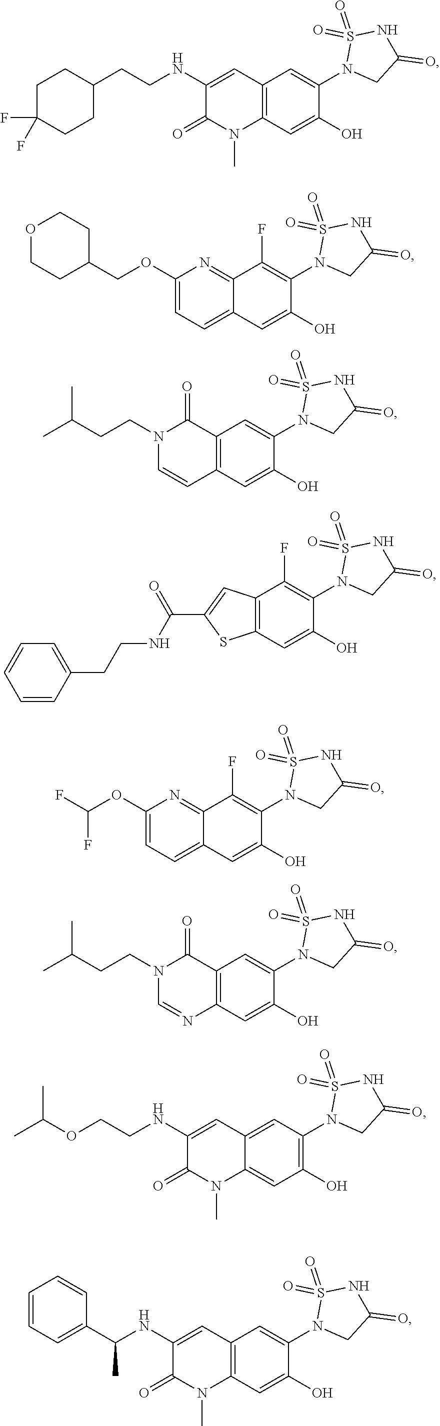

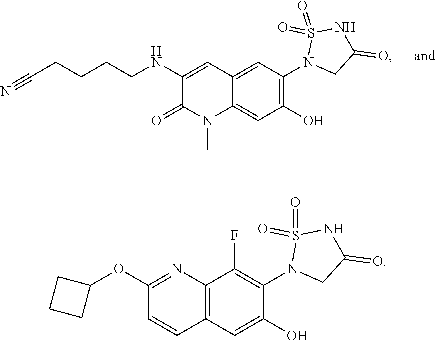 [SMILES: Cn1c(=O)c(NCCCCC#N)cc2cc(N3CC(=O)NS3(=O)=O)c(O)cc21 ; "and" ; O=C1CN(c2c(O)cc3ccc(OC4CCC4)nc3c2F)S(=O)(=O)N1]

In an aspect is provided a pharmaceutical composition comprising a compound described herein, or a pharmaceutically acceptable salt or solvate thereof, and a pharmaceutically acceptable excipient.

In an aspect is provided a method of treating cancer in a subject in need thereof, comprising administering to the subject a therapeutically effective amount of a compound described herein, or a pharmaceutically acceptable salt or solvate thereof.

In an aspect is provided a method of potentiating immunity of a cell, comprising: contacting the cell with a compound described herein, thereby potentiating immunity of the cell,
wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.

In an aspect is provided a method of potentiating immunity of a cell, comprising:
(a) contacting the cell with a compound described herein; and
(b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell.

In some embodiments, the cell is a lymphoid cell.

In some embodiments, the method further comprises administering the cell to a subject in need thereof.

In some embodiments, the method further comprises administering the compound described herein to the subject prior to, concurrent with, or subsequent to the administering the cell.

In some embodiments, prior to the administering the compound described herein, a cell of the subject exhibits expression or activity of PTPN2.

In an aspect is provided a method of potentiating immunity of a subject in need thereof, comprising:
(a) selecting the subject that exhibits expression or activity of PTPN2; and
(b) downregulating expression or activity of PTPN2 by introducing a compound described herein to a cell of the subject, thereby potentiating immunity of the subject.

In an aspect is provided a method of potentiating immunity of a subject in need thereof, comprising:
administering a lymphoid cell to the subject; and
administering a compound described herein to the subject, thereby potentiating immunity of the subject.

In an aspect is provided a method of potentiating anti-tumor or anti-cancer immunity of a subject in need thereof, comprising:
(a) contacting a lymphoid cell of the subject with a compound described herein, thereby potentiating the anti-tumor or anti-cancer immunity of the subject.

In an aspect is provided a method of treating tumor or cancer of a subject in need thereof, comprising:
(a) contacting a lymphoid cell of the subject with a compound described herein, thereby treating the tumor or cancer of the subject.

In an aspect is provided a method of treating tumor or cancer of a subject in need thereof comprising administering to said subject an effective amount of a compound described herein and an additional agent selected from the group consisting of a chemotherapeutic agent and an immune modulator.

In an aspect is provided a method of treating tumor or cancer of a subject in need thereof comprising administering to said subject an effective amount of a compound described herein in conjunction with a cell therapy.

In an aspect, provided is a compound of Formula (I), or a pharmaceutically acceptable salt or solvate thereof:

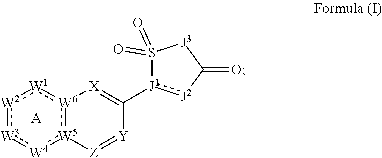

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein at least one of W¹, W², W³, W⁴, W⁵, and W⁶ is N, N(R¹), N(R²), N(R³), N(R⁴), or O; and
X is N or C(R⁵);
Y is N or C(R⁶);
Z is N or C(R⁷);
J¹ is N, C, or C(R¹);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
R^{12a} is selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}; each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl
[symbol] indicates a single or double bond such that all valences are satisfied; and provided that:
   i) at least two of Ring A [symbol] are double bonds;
   ii) when J³ is CH₂; R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}; and
   iii) the compound is not

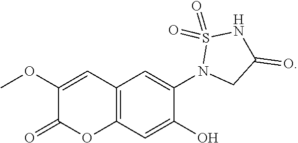 [SMILES: COc1cc2cc(N3CC(=O)NS3(=O)=O)c(O)cc2oc1=O]

In some embodiments of the compound, or a pharmaceutically acceptable salt or solvate thereof, X is C(R⁵). In some embodiments of the compound, or a pharmaceutically acceptable salt or solvate thereof, Y is C(R⁶). In some embodiments of the compound, or a pharmaceutically acceptable salt or solvate thereof, Z is C(R⁷). In some embodiments of the compound, or a pharmaceutically acceptable salt or solvate thereof, J¹ is N and J² is C(R⁹)(R^{9a}). In some embodiments of the compound, or a pharmaceutically acceptable salt or solvate thereof, J¹ is C(R⁸) and J² is C(R⁹)(R^{9a}). In some embodiments of the compound, or a pharmaceutically acceptable salt or solvate thereof, J² is CH₂. In some embodiments of the compound, or a pharmaceutically acceptable salt or solvate thereof, J¹ is C and J² is C(R⁹). In some embodiments of the compound, or a pharmaceutically acceptable salt or solvate thereof, J² is C(H). In some embodiments of the compound, or a pharmaceutically acceptable salt or solvate thereof, wherein J³ is N(R¹⁰). In some embodiments of the compound, or a pharmaceutically acceptable salt or solvate thereof, J³ is N(H). In some embodiments of the compound, or a pharmaceutically acceptable salt or solvate thereof, the compound has the structure of Formula (Ia):

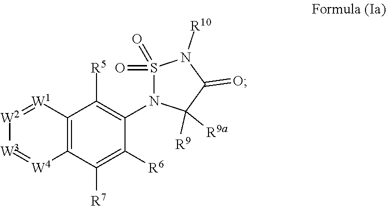

wherein at least one of W¹, W², W³, and W⁴ is N.

In some embodiments of the compound, or a pharmaceutically acceptable salt or solvate thereof, the compound has the structure of Formula (Ib):

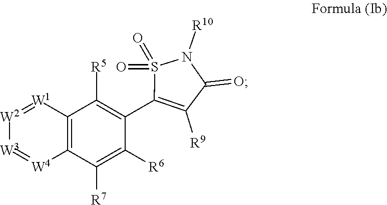

wherein at least one of W¹, W², W³, and W⁴ is N.

In some embodiments of the compound, or a pharmaceutically acceptable salt or solvate thereof, the compound has the structure of Formula (Ic):

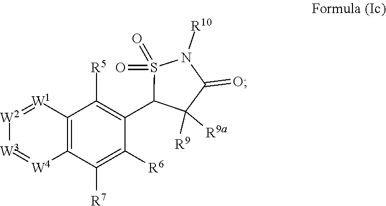

wherein at least one of W¹, W², W³, and W⁴ is N.

In some embodiments of the compound, or a pharmaceutically acceptable salt or solvate thereof, W¹ is N, W² is C(R²), W³ is C(R³), and W⁴ is C(R⁴). In some embodiments of the compound, or a pharmaceutically acceptable salt or solvate thereof, W¹ is C(R¹), W² is N, W³ is C(R³), and W⁴ is C(R⁴). In some embodiments of the compound, or a pharmaceutically acceptable salt or solvate thereof, W¹ is C(R¹), W² is C(R²), W³ is N, and W⁴ is C(R⁴). In some embodiments of the compound, or a pharmaceutically acceptable salt or solvate thereof, W¹ is C(R¹), W² is C(R²), W³ is C(R³), and W⁴ is N.

In some embodiments of the compound, or a pharmaceutically acceptable salt or solvate thereof, the compound has the structure of Formula (Ig):

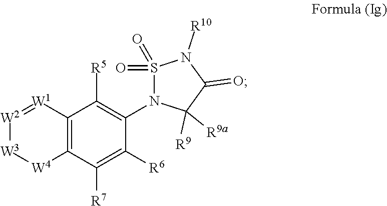

wherein at least one of W¹, W², W³, and W⁴ is N, N(R³), N(R⁴), or O.

In some embodiments of the compound, or a pharmaceutically acceptable salt or solvate thereof, the compound has the structure of Formula (Ih):

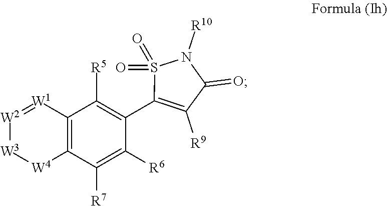

wherein at least one of W¹, W², W³, and W⁴ is N, N(R³), N(R⁴), or O.

In some embodiments of the compound, or a pharmaceutically acceptable salt or solvate thereof, the compound has the structure of Formula (Ii):

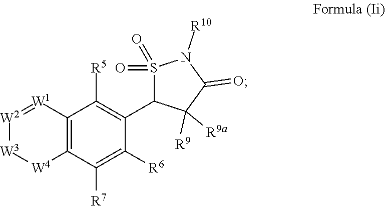

wherein at least one of W¹, W², W³, and W⁴ is N, N(R³), N(R⁴), or O.

In some embodiments of the compound, or a pharmaceutically acceptable salt or solvate thereof, W¹ is C(R¹) and W² is C(R²). In some embodiments of the compound, or a pharmaceutically acceptable salt or solvate thereof, W³ is N(R³) and W⁴ is C(O). In some embodiments of the compound, or a pharmaceutically acceptable salt or solvate thereof, W³ is C(O) and W⁴ is N(R⁴). In some embodiments of the compound, or a pharmaceutically acceptable salt or solvate thereof, W³ is O and W⁴ is C(R⁴)(R^{4a}).

In some embodiments of the compound, or a pharmaceutically acceptable salt or solvate thereof, the compound has the structure of Formula (Ij):

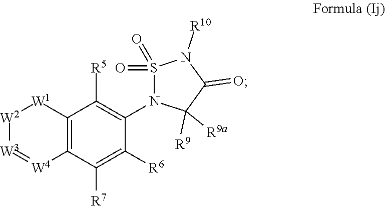

wherein at least one of W¹, W², W³, and W⁴ is N(R¹), N(R²), N, or O.

In some embodiments of the compound, or a pharmaceutically acceptable salt or solvate thereof, the compound has the structure of Formula (Ik):

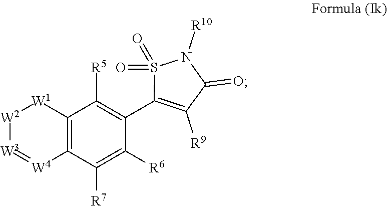

wherein at least one of W¹, W², W³, and W⁴ is N(R¹), N(R²), N, or O.

In some embodiments of the compound, or a pharmaceutically acceptable salt or solvate thereof, the compound has the structure of Formula (Im):

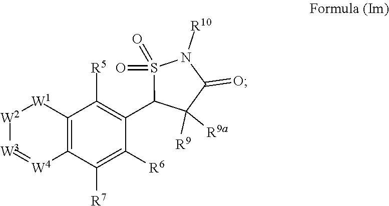

wherein at least one of W¹, W², W³, and W⁴ is N, N(R³), N(R⁴), or O.

In some embodiments of the compound, or a pharmaceutically acceptable salt or solvate thereof, W¹ is C(R¹)(R^{1a}), W² is O, W³ is C(R³), and W⁴ is C(R⁴). In some embodiments of the compound, or a pharmaceutically acceptable salt or solvate thereof, W¹ is CO, N(R1) or C(R¹)(R^{1a}); W² is CO, N(R2), or C(R²)(R^{2a}); W³ is N, or C(R³); and W⁴ is N, or C(R⁴).

In some embodiments of the compound, or a pharmaceutically acceptable salt or solvate thereof, the compound has the structure of Formula (Iv):

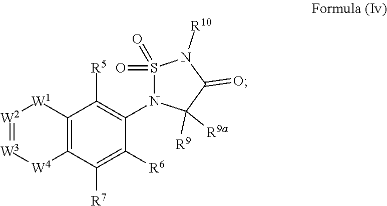

wherein at least one of W¹, W², W³, and W⁴ is N(R¹), N(R⁴), N, or O.

In some embodiments of the compound, or a pharmaceutically acceptable salt or solvate thereof, the compound has the structure of Formula (Iw):

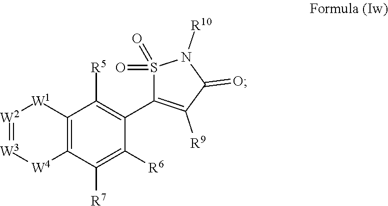

wherein at least one of W¹, W², W³, and W⁴ is N(R¹), N(R⁴), N, or O.

In some embodiments of the compound, or a pharmaceutically acceptable salt or solvate thereof, the compound has the structure of Formula (Ix):

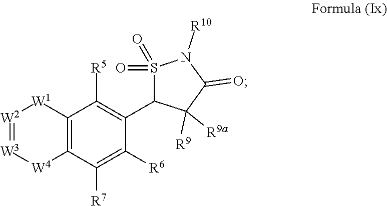

wherein at least one of W¹, W², W³, and W⁴ is N(R¹), N(R⁴), N, or O.

In some embodiments of the compound, or a pharmaceutically acceptable salt or solvate thereof, the compound has the structure of Formula (Io):

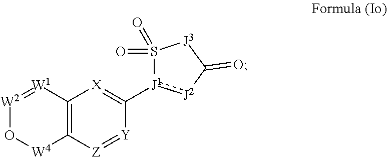

wherein W¹ is C(R¹); W² is C(R²); and W⁴ is C(R⁴)(R^{4a}), C(O), S(O), S(O)₂, S(O)(NR⁴), S(O)(R⁴), or P(O)(R⁴).

In some embodiments of the compound, or a pharmaceutically acceptable salt or solvate thereof, the compound has the structure of Formula (Ioa):

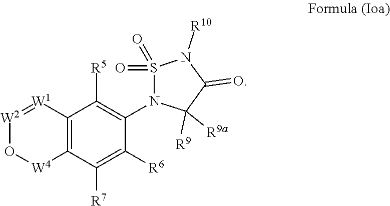

In some embodiments of the compound, or a pharmaceutically acceptable salt or solvate thereof, the compound has the structure of Formula (Iob):

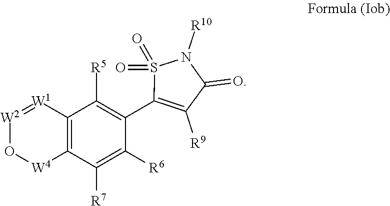

In some embodiments of the compound, or a pharmaceutically acceptable salt or solvate thereof, the compound has the structure of Formula (Ic):

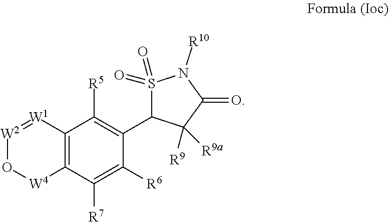

In some embodiments of the compound, or a pharmaceutically acceptable salt or solvate thereof, the compound has the structure of Formula (Ip):

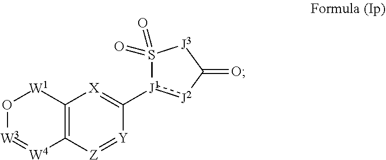

wherein: W¹ is C(R¹)(R^{1a}), C(O), S(O), S(O)₂, S(O)(NR¹), S(O)(R¹), or P(O)(R¹); W³ is C(R³); and W⁴ is C(R⁴).

In some embodiments of the compound, or a pharmaceutically acceptable salt or solvate thereof, the compound has the structure of Formula (Ipa):

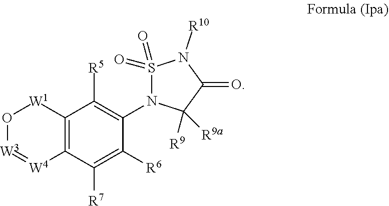

In some embodiments of the compound, or a pharmaceutically acceptable salt or solvate thereof, the compound has the structure of Formula (Ipb):

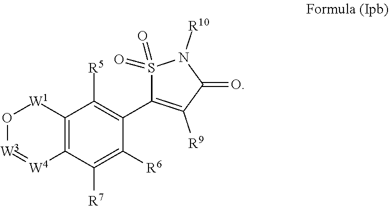

In some embodiments of the compound, or a pharmaceutically acceptable salt or solvate thereof, the compound has the structure of Formula (Ipc):

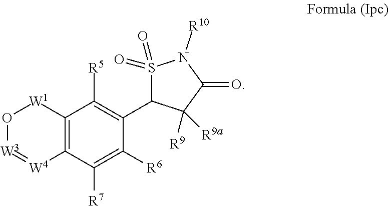

In some embodiments of the compound, or a pharmaceutically acceptable salt or solvate thereof, the compound has the structure of Formula (Ipd):

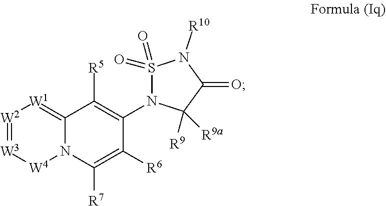

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R), or P(O)(R⁴).

In some embodiments of the compound, or a pharmaceutically acceptable salt or solvate thereof, the compound has the structure of Formula (Iq):

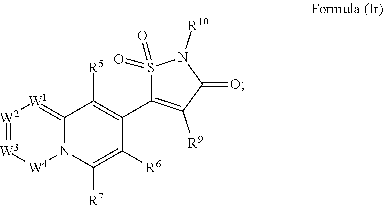

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R), or P(O)(R⁴).

In some embodiments of the compound, or a pharmaceutically acceptable salt or solvate thereof, the compound has the structure of Formula (Ir):

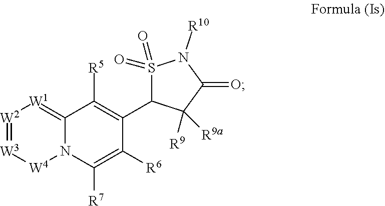

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R), or P(O)(R⁴).

In some embodiments of the compound, or a pharmaceutically acceptable salt or solvate thereof, W⁴ is C(O).

In an aspect, provided is a compound of Formula (III), or a pharmaceutically acceptable salt or solvate thereof:

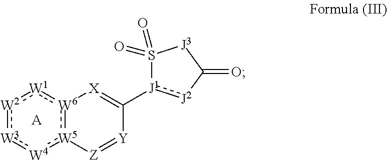

wherein:
W¹ is N, N(R¹), O, C(R), C(R¹)(R^{1a}), C(O), S(O), S(O)₂, S(O)(NR¹), S(O)(R¹), or P(O)(R¹);
W² is N, N(R²), O, C(R²), C(R²)(R^{2a}), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein
   i) at least one of W¹, W², W³, W⁴, W⁵, and W⁶ is N, N(R¹), N(R²), N(R³), N(R⁴), or O; and
   ii) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R);
X is N or C(R⁵);
Y is N or C(R⁶);
Z is N or C(R¹)
J¹ is N, C, or C(R¹);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁_.heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and provided the compound is not

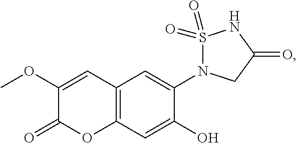 [SMILES: COc1cc2cc(N3CC(=O)NS3(=O)=O)c(O)cc2oc1=O]

In some embodiments of the compound, or a pharmaceutically acceptable salt or solvate thereof, X is C(R⁵). In some embodiments of the compound, or a pharmaceutically acceptable salt or solvate thereof, Y is C(R⁶). In some embodiments of the compound, or a pharmaceutically acceptable salt or solvate thereof, Z is C(R⁷). In some embodiments of the compound, or a pharmaceutically acceptable salt or solvate thereof, J¹ is N and J² is C(R⁹)(R^{9a}). In some embodiments of the compound, or a pharmaceutically acceptable salt or solvate thereof, J¹ is C(R⁸) and J² is C(R⁹)(R^{9a}). In some embodiments of the compound, or a pharmaceutically acceptable salt or solvate thereof, J² is CH₂. In some embodiments of the compound, or a pharmaceutically acceptable salt or solvate thereof, J¹ is C and J² is C(R⁹). In some embodiments of the compound, or a pharmaceutically acceptable salt or solvate thereof, J² is C(H). In some embodiments of the compound, or a pharmaceutically acceptable salt or solvate thereof, J³ is N(R¹⁰). In some embodiments of the compound, or a pharmaceutically acceptable salt or solvate thereof, J³ is N(H).

In some embodiments of the compound, or a pharmaceutically acceptable salt or solvate thereof, the compound has the structure of Formula (IIIa):

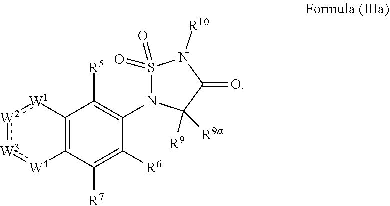

In some embodiments of the compound, or a pharmaceutically acceptable salt or solvate thereof, the compound has the structure of Formula (IIIb):

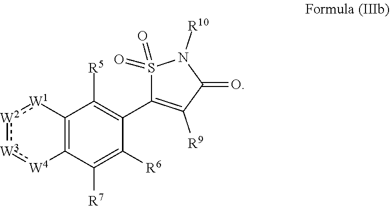

In some embodiments of the compound, or a pharmaceutically acceptable salt or solvate thereof, the compound has the structure of Formula (IIIc):

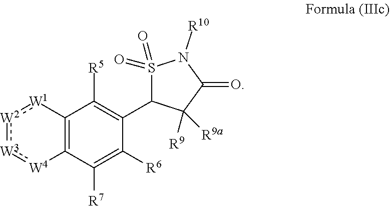

In some embodiments of the compound, or a pharmaceutically acceptable salt or solvate thereof, W¹ is C(O). In some embodiments of the compound, or a pharmaceutically acceptable salt or solvate thereof, W² is N(R²). In some embodiments of the compound, or a pharmaceutically acceptable salt or solvate thereof, W³ and W⁴ are connected by a double bond. In some embodiments of the compound, or a pharmaceutically acceptable salt or solvate thereof, W² is C(O). In some embodiments of the compound, or a pharmaceutically acceptable salt or solvate thereof, W¹ is N(R¹) and W³ and W⁴ are connected by a double bond. In some embodiments of the compound, or a pharmaceutically acceptable salt or solvate thereof, W³ is C(O). In some embodiments of the compound, or a pharmaceutically acceptable salt or solvate thereof, W² is N(R²) and W¹ is C(R¹)(R^{1a}). In some embodiments of the compound, or a pharmaceutically acceptable salt or solvate thereof, W⁴ is N(R⁴). In some embodiments of the compound, or a pharmaceutically acceptable salt or solvate thereof, W⁴ is C(R⁴)(R^{4a}). In some embodiments of the compound, or a pharmaceutically acceptable salt or solvate thereof, W⁴ is N(R⁴) and W¹ and W² are connected by a double bond. In some embodiments of the compound, or a pharmaceutically acceptable salt or solvate thereof, W⁴ is C(O). In some embodiments of the compound, or a pharmaceutically acceptable salt or solvate thereof, W¹ is O. In some embodiments of the compound, or a pharmaceutically acceptable salt or solvate thereof, W² is C(R²)(R^{2a}) and W³ is C(R³)(R^{3a}). In some embodiments of the compound, or a pharmaceutically acceptable salt or solvate thereof, W² is C(R²) and W³ is C(R³).

In some embodiments of the compound, or a pharmaceutically acceptable salt or solvate thereof, the compound has the structure of Formula (IIId):

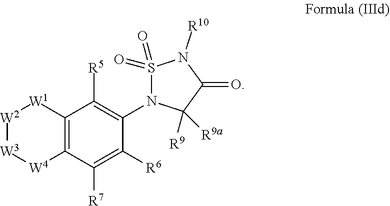

In some embodiments of the compound, or a pharmaceutically acceptable salt or solvate thereof, the compound has the structure of Formula (IIIe):

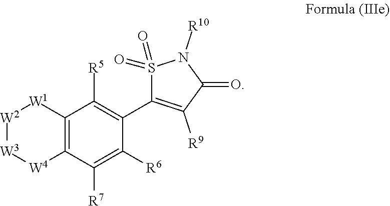

In some embodiments of the compound, or a pharmaceutically acceptable salt or solvate thereof, the compound has the structure of Formula (IIIf):

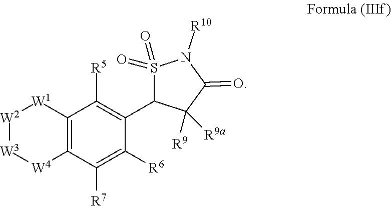

In some embodiments of the compound, or a pharmaceutically acceptable salt or solvate thereof, the compound has the structure of Formula (IIIg):

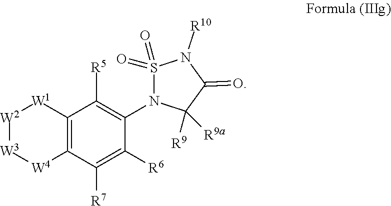

In some embodiments of the compound, or a pharmaceutically acceptable salt or solvate thereof, the compound has the structure of Formula (IIIh):

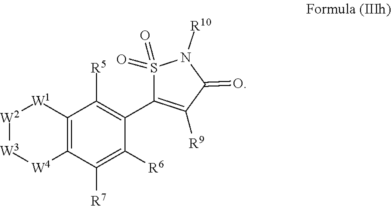

In some embodiments of the compound, or a pharmaceutically acceptable salt or solvate thereof, the compound has the structure of Formula (IIIi):

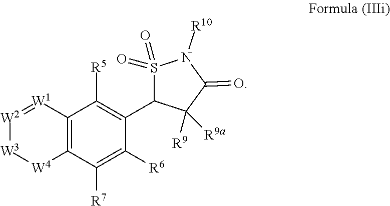

In some embodiments of the compound, or a pharmaceutically acceptable salt or solvate thereof, the compound has the structure of Formula (IIIj):

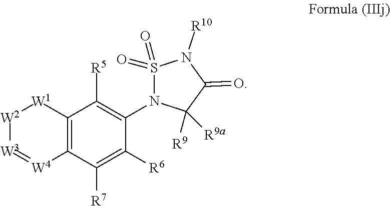

In some embodiments of the compound, or a pharmaceutically acceptable salt or solvate thereof, the compound has the structure of Formula (IIIk):

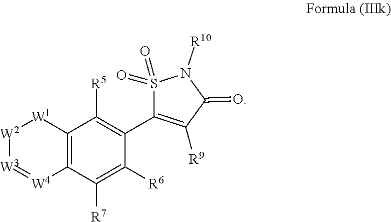

In some embodiments of the compound, or a pharmaceutically acceptable salt or solvate thereof, the compound has the structure of Formula (IIIl):

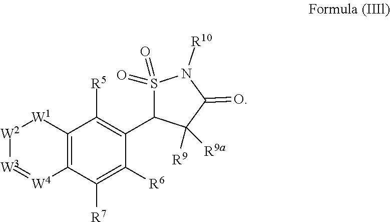

In some embodiments of the compound, or a pharmaceutically acceptable salt or solvate thereof, the compound has the structure of Formula (IIIm):

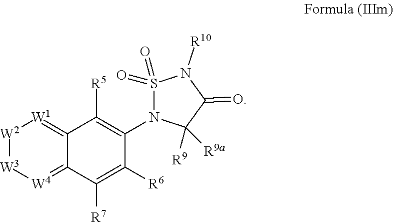

In some embodiments of the compound, or a pharmaceutically acceptable salt or solvate thereof, the compound has the structure of Formula (IIIn):

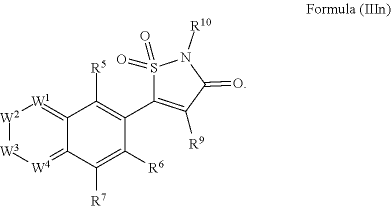

In some embodiments of the compound, or a pharmaceutically acceptable salt or solvate thereof, the compound has the structure of Formula (IIIo):

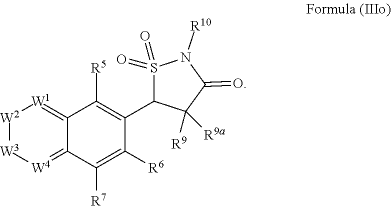

In some embodiments of the compound, or a pharmaceutically acceptable salt or solvate thereof, the compound has the structure of Formula (IIIp):

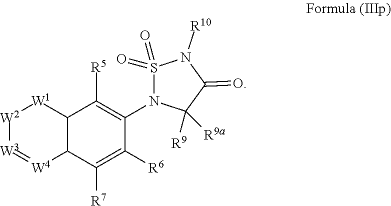

In some embodiments of the compound, or a pharmaceutically acceptable salt or solvate thereof, the compound has the structure of Formula (IIIq):

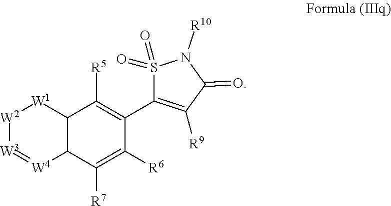

In some embodiments of the compound, or a pharmaceutically acceptable salt or solvate thereof, the compound has the structure of Formula (IIIr):

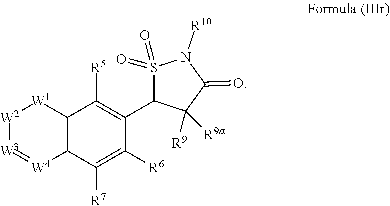

In some embodiments of the compound, or a pharmaceutically acceptable salt or solvate thereof, the compound has the structure of Formula (IIIs):

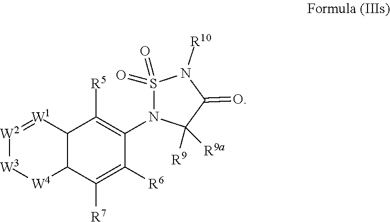

In some embodiments of the compound, or a pharmaceutically acceptable salt or solvate thereof, the compound has the structure of Formula (IIIt):

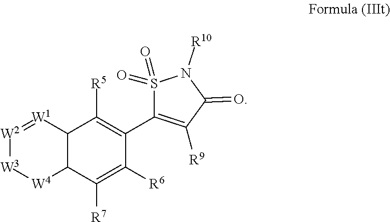

In some embodiments of the compound, or a pharmaceutically acceptable salt or solvate thereof, the compound has the structure of Formula (IIIu):

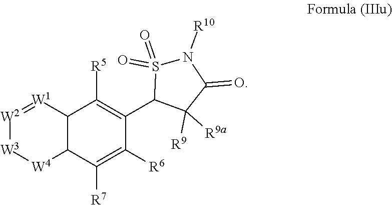

In an aspect, provided is a compound of Formula (II), or a pharmaceutically acceptable salt or solvate thereof:

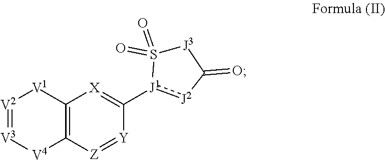

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl.

In some embodiments of the compound, or a pharmaceutically acceptable salt or solvate thereof, X is C(R⁵). In some embodiments of the compound, or a pharmaceutically acceptable salt or solvate thereof, Y is C(R⁶). In some embodiments of the compound, or a pharmaceutically acceptable salt or solvate thereof, Z is C(R⁷). In some embodiments of the compound, or a pharmaceutically acceptable salt or solvate thereof, J¹ is N and J² is C(RY)(R^{9a}). In some embodiments of the compound, or a pharmaceutically acceptable salt or solvate thereof, J¹ is C(R⁸) and J² is C(R⁹)(R^{9a}). In some embodiments of the compound, or a pharmaceutically acceptable salt or solvate thereof, J² is CH₂. In some embodiments of the compound, or a pharmaceutically acceptable salt or solvate thereof, J¹ is C and J² is C(R⁷). In some embodiments of the compound, or a pharmaceutically acceptable salt or solvate thereof, J² is C(H). In some embodiments of the compound, or a pharmaceutically acceptable salt or solvate thereof, J³ is N(R¹⁰). In some embodiments of the compound, or a pharmaceutically acceptable salt or solvate thereof, J³ is N(H). In some embodiments of the compound, or a pharmaceutically acceptable salt or solvate thereof, V¹ is a bond and V⁴ is S. In some embodiments of the compound, or a pharmaceutically acceptable salt or solvate thereof, V¹ is S and V⁴ is a bond.

In some embodiments of the compound, or a pharmaceutically acceptable salt or solvate thereof, the compound has the structure of Formula (IIa):

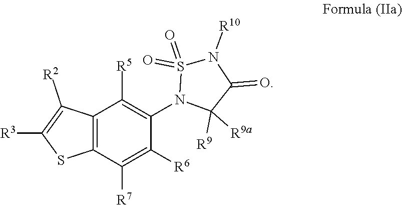

In some embodiments of the compound, or a pharmaceutically acceptable salt or solvate thereof, the compound has the structure of Formula (IIb):

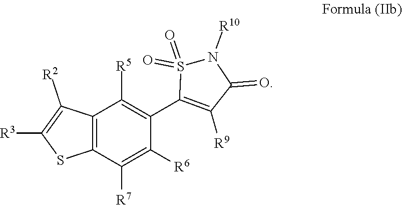

In some embodiments of the compound, or a pharmaceutically acceptable salt or solvate thereof, the compound has the structure of Formula (IIc):

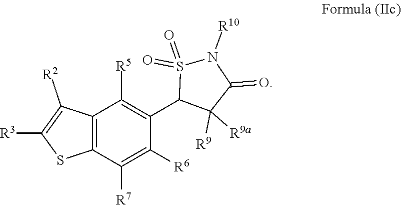

In some embodiments of the compound, or a pharmaceutically acceptable salt or solvate thereof, the compound has the structure of Formula (IId):

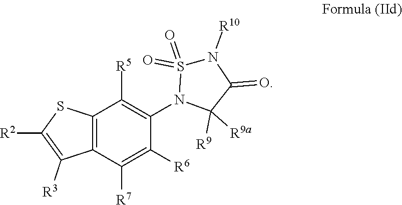

In some embodiments of the compound, or a pharmaceutically acceptable salt or solvate thereof, the compound has the structure of Formula (IIe):

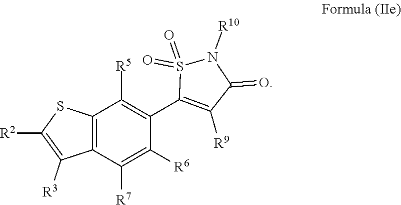

In some embodiments of the compound, or a pharmaceutically acceptable salt or solvate thereof, the compound has the structure of Formula (IIf):

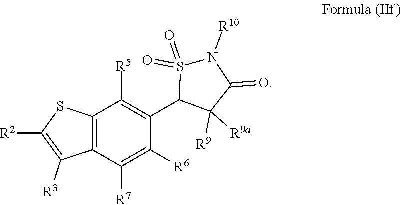

In some embodiments of the compound, or a pharmaceutically acceptable salt or solvate thereof, R⁵ is selected from hydrogen, halogen, and C₁₋₆alkyl optionally substituted with one, two, or three R^{20e}.

In some embodiments of the compound, or a pharmaceutically acceptable salt or solvate thereof, R⁵ is selected from hydrogen and halogen.

In some embodiments of the compound, or a pharmaceutically acceptable salt or solvate thereof, R⁶ is selected from hydrogen, halogen, —OR^{12a}, and C₁₋₆alkyl optionally substituted with one, two, or three R^{20f}.

In some embodiments of the compound, or a pharmaceutically acceptable salt or solvate thereof, R⁶ is —OH.

In some embodiments of the compound, or a pharmaceutically acceptable salt or solvate thereof, R⁷ is selected from hydrogen, halogen, and C₁₋₆alkyl optionally substituted with one, two, or three R^{20g}.

In some embodiments of the compound, or a pharmaceutically acceptable salt or solvate thereof, R⁷ is hydrogen.

In some embodiments of the compound, or a pharmaceutically acceptable salt or solvate thereof, R⁹ is hydrogen.

In some embodiments of the compound, or a pharmaceutically acceptable salt or solvate thereof, R^{9a} is hydrogen.

In some embodiments of the compound, or a pharmaceutically acceptable salt or solvate thereof, R¹⁰ is hydrogen.

In some embodiments of the compound, or a pharmaceutically acceptable salt or solvate thereof, R² is selected from hydrogen, halogen, C₁₋₆alkyl, C₂₋₉heterocycloalkyl, C₁₋₉heteroaryl, —OR¹², —SR¹², —N(R¹²)(R¹³), —C(O)R¹⁵, —C(O)N(R¹²)(R¹³), —S(O)₂R¹⁵, and —S(O)₂N(R¹²)(R¹³)—, wherein C₁₋₆alkyl, C₂₋₉heterocycloalkyl, and C₁₋₉heteroaryl are optionally substituted with one, two, or three R^{20b}.

In some embodiments of the compound, or a pharmaceutically acceptable salt or solvate thereof, R² is selected from C₁₋₆ alkyl, C₂₋₉heterocycloalkyl, C₁₋₉heteroaryl, —OR¹², —N(R¹²)(R¹³), —C(O)R¹⁵, —C(O)N(R¹²)(R¹³), wherein C₁₋₆alkyl, C₂₋₉heterocycloalkyl, and C₁₋₉heteroaryl are optionally substituted with one, two, or three R^{20b}.

In some embodiments of the compound, or a pharmaceutically acceptable salt or solvate thereof, R² is selected from —OR¹², N(R¹²)(R¹³), —C(O)R¹⁵, —C(O)N(R¹²)(R¹³); R¹³ is hydrogen;
R¹² and R¹⁵ are independently

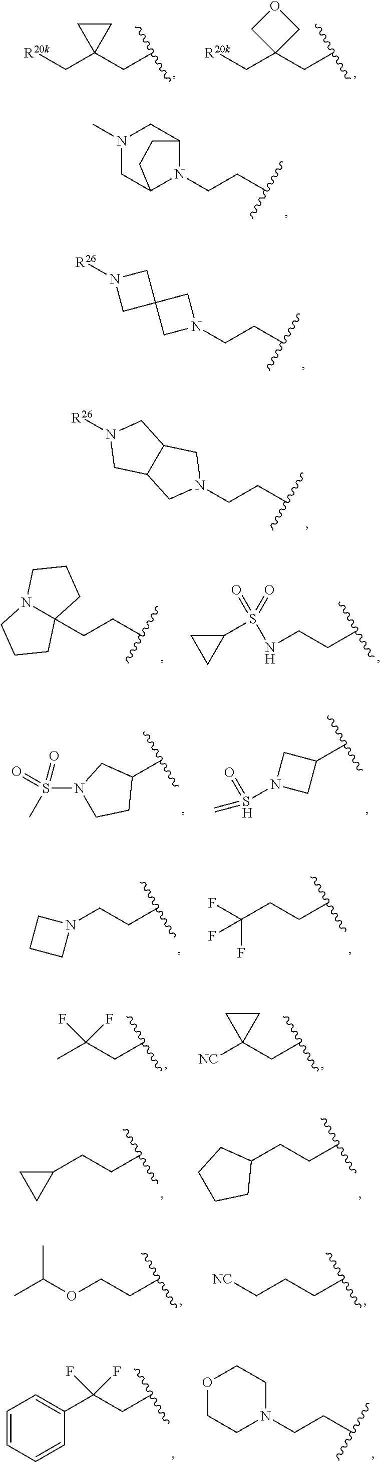

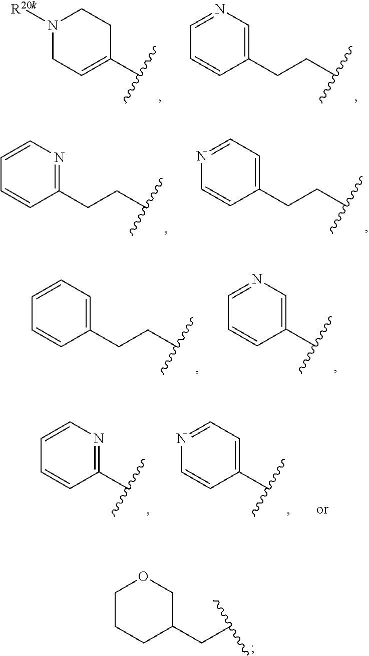

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments of the compound, or a pharmaceutically acceptable salt or solvate thereof, R² is selected from —OR¹², N(R¹²)(R¹³), —C(O)R¹⁵, —C(O)N(R¹²)(R¹³);
R¹³ is hydrogen;
R¹² and R¹⁵ are independently

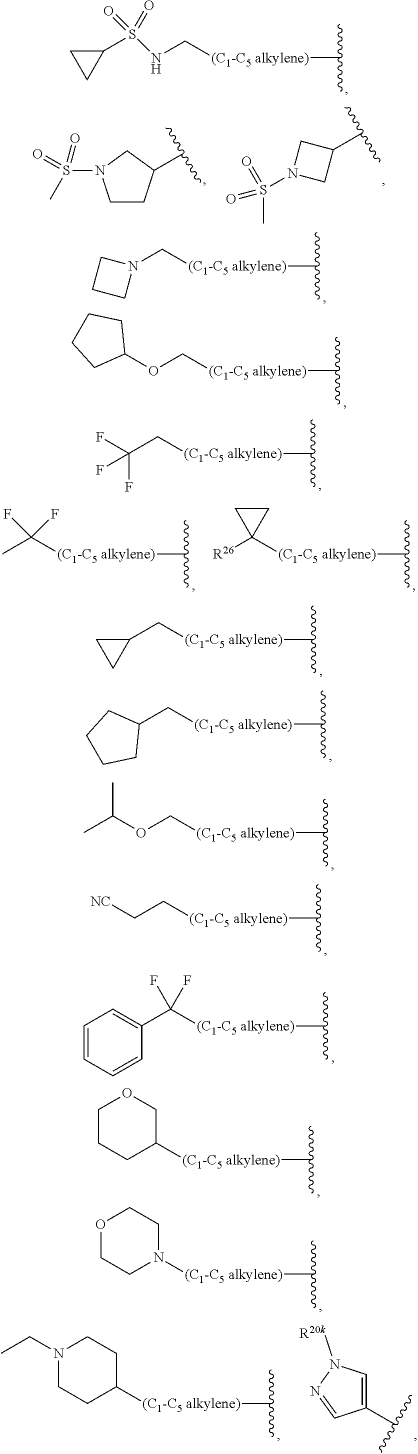

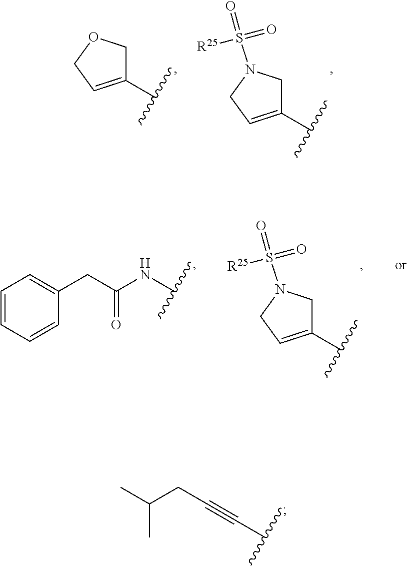

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments of the compound, or a pharmaceutically acceptable salt or solvate thereof,
R²⁶ is selected from

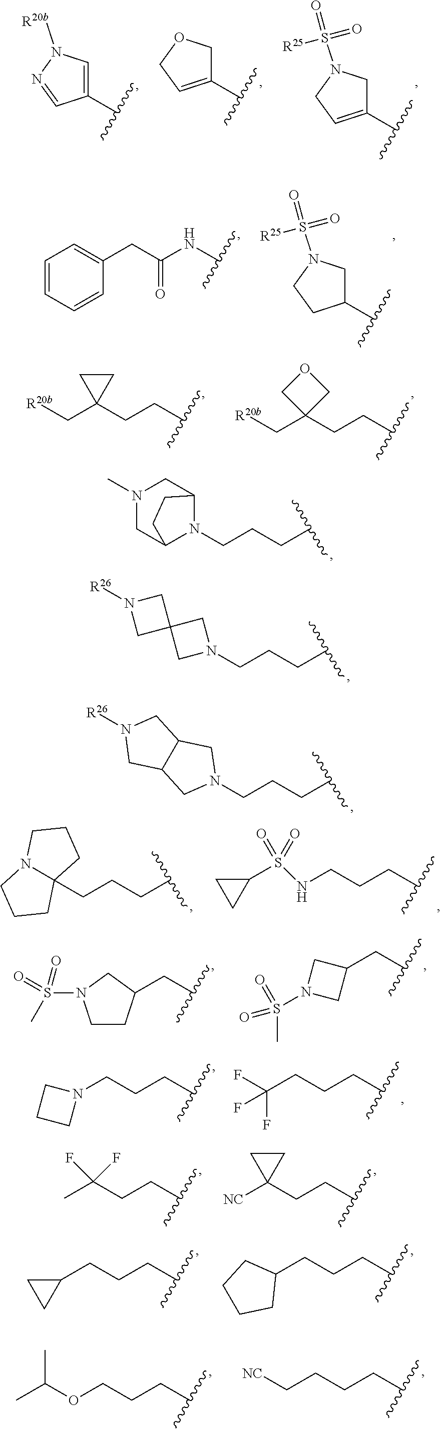

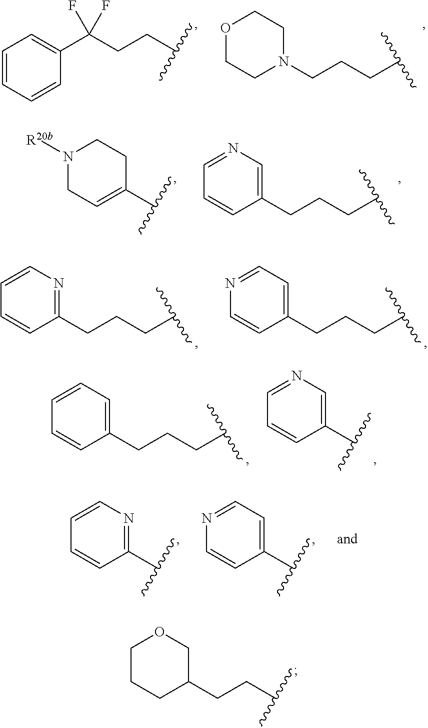

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. n

In some embodiments of the compound, or a pharmaceutically acceptable salt or solvate thereof,

R² is independently

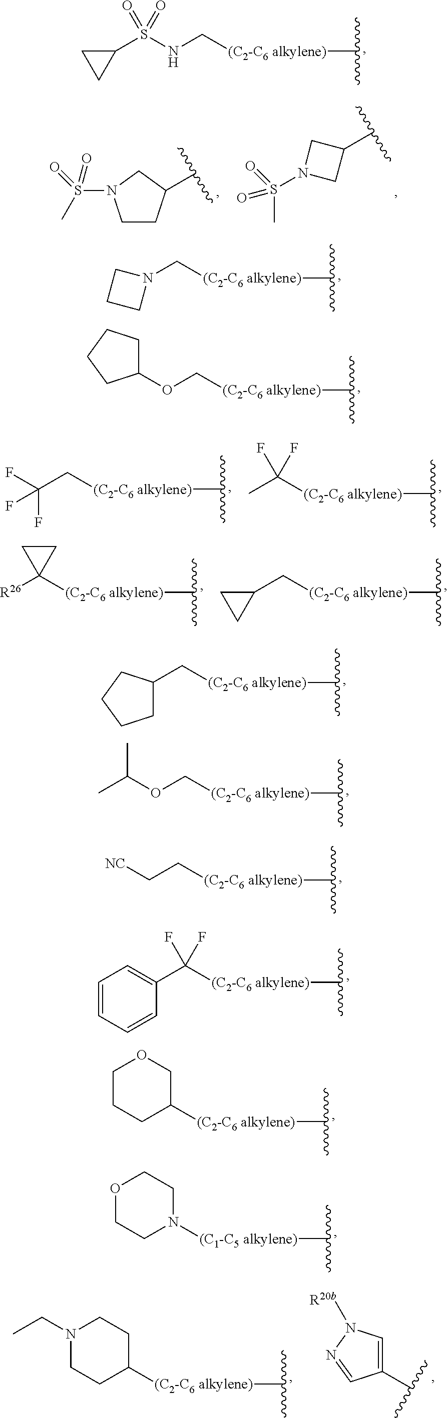

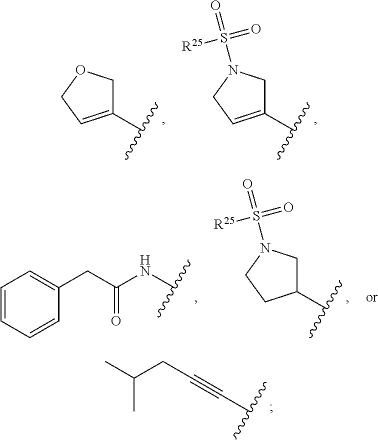

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments of the compound, or a pharmaceutically acceptable salt or solvate thereof, R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}. In some embodiments of the compound, or a pharmaceutically acceptable salt or solvate thereof, R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}.

In an aspect, provided is a compound, or a pharmaceutically acceptable salt or solvate thereof, selected from:

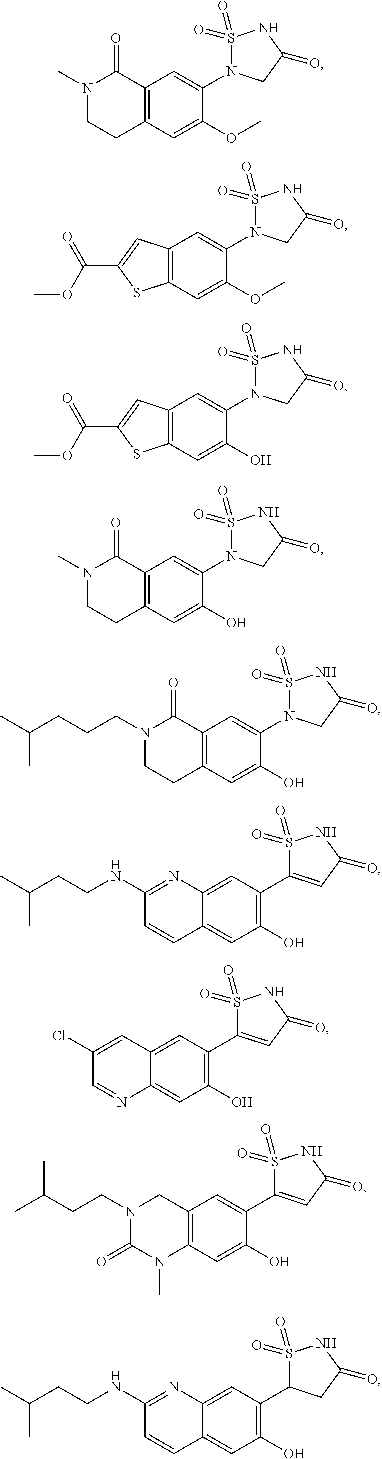

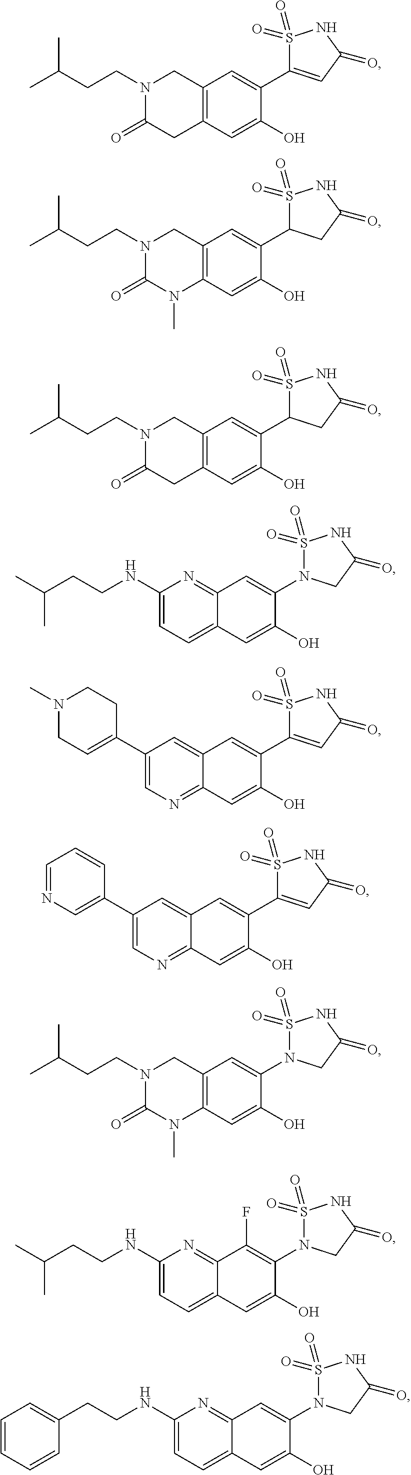

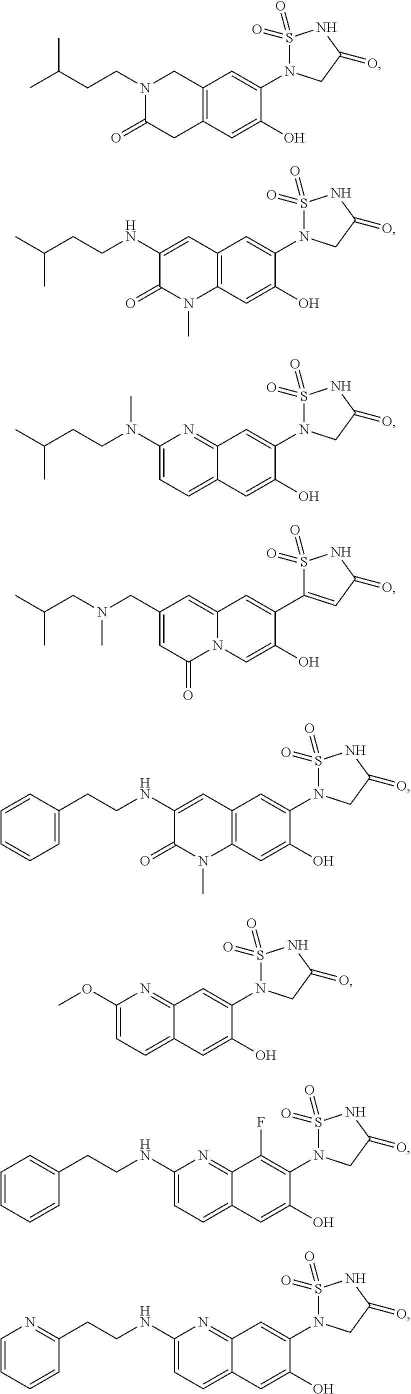

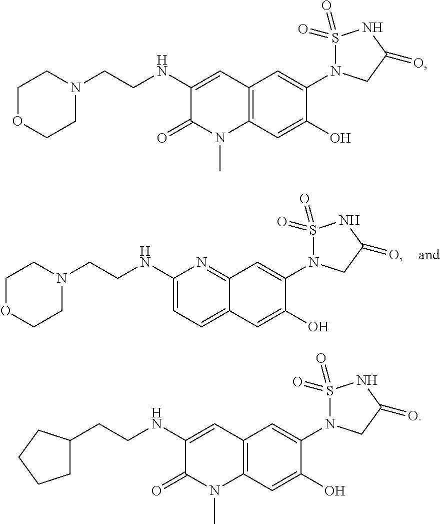

In an aspect, provided is a pharmaceutical composition comprising a compound described herein, or a pharmaceutically acceptable salt or solvate thereof, and a pharmaceutically acceptable excipient.

In an aspect, provided is a method of treating cancer in a subject in need thereof, comprising administering to the subject a therapeutically effective amount of a compound described herein, including in embodiments thereof, or a pharmaceutically acceptable salt or solvate thereof.

In an aspect, provided is a method of potentiating immunity of a cell, comprising: (a) contacting the cell with a compound described herein, including in embodiments thereof, thereby potentiating immunity of the cell, wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.

In an aspect, provided is a method of potentiating immunity of a cell, comprising: (a) contacting the cell with a compound described herein, including in embodiments; and (b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell.

In some embodiments of a method described herein, including in embodiments thereof, (a) is performed prior to, concurrent with, or subsequent to (b). In some embodiments, the cell (e.g., lymphoid cell) retains expression or activity of PTPN2 prior to (a).

In some embodiments, a subject method further comprises administering the cell to a subject in need thereof. In some embodiments, a subject method further comprises administering a compound described herein, including in embodiments thereof, to the subject prior to, concurrent with, or subsequent to the administering the cell. In some embodiment, a cell of the subject exhibits expression or activity of PTPN2.

In an aspect, provided is a method of potentiating immunity of a subject in need thereof, comprising: administering a lymphoid cell to the subject, thereby potentiating immunity of the subject, wherein expression or activity of PTPN2 in the lymphoid cell is transiently downregulated.

In some embodiments of a method described herein, including in embodiments thereof, the method further comprises transiently downregulating the expression or activity of PTPN2 in the lymphoid cell. Where desired, the transiently downregulating is performed once, or performed intermittently for a desired number of times, such as two or more times. In some embodiments, the transiently downregulating comprises introducing a compound described herein, including in embodiments thereof, to a cell of interest. In some embodiments, a first intermittent dosing regimen of a compound described herein and a second intermittent dosing regimen of the compound is the same or different. In some embodiments, prior to the transiently downregulating, a lymphoid cell being contacted by a subject compound exhibits expression or activity of PTPN2. In some embodiments of a method described herein, including in embodiments thereof, the lymphoid cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments, the method further comprises administering a compound described herein to the subject prior to, concurrent with, or subsequent to the administering the lymphoid cell. In some embodiment, the subject exhibits expression or activity of PTPN2. In an aspect, provided is a method of potentiating immunity of a subject in need thereof, comprising: (a) selecting the subject that exhibits expression or activity of PTPN2; and (b) downregulating expression or activity of PTPN2 in a cell of the subject, thereby potentiating immunity of the subject. In some embodiments, a method described herein, the step (b) is performed in vivo or ex vivo. In some embodiments of a method described herein, the downregulating comprises introducing a compound described herein, to the cell. Where desired, the downregulating comprises transiently downregulating the expression or activity of PTPN2. In some embodiments, the transiently downregulating is performed once or multiple times such as two, three or more times. In some embodiments, a first intermittent dosing regimen of a compound described herein is the same as a second intermittent dosing regimen utilizing a the compound disclosed herein. In some embodiments, the first intermittent dosing regimen of a compound described herein is different from that of a second intermittent dosing regimen. In practicing any of the aforementioned methods, a cell of the subject being treated can be a lymphoid cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments, the cell of the subject does not exhibit a mutation of (i) a first gene encoding PTPN2 or (ii) a second gene operatively linked to PTPN2, wherein the mutation inhibits the expression and/or activity of PTPN2. In some embodiments, a method described herein further comprises selecting a subject that exhibits expression or activity of PTPN2. Where desired the step of selecting comprises performing a nucleic acid assay using at least a portion of a genome or transcriptome of the cell of the subject to detect the mutation. In some embodiments, the selecting comprises performing a protein assay to detect a functionally active PTPN2 or a functionally inactive PTPN2.

In an aspect, provided is a method of potentiating immunity of a subject in need thereof, comprising: administering a lymphoid cell to the subject; and administering a compound described herein to the subject, thereby potentiating immunity of the subject. Where desired, the administering the compound described herein can be performed prior to, concurrent with, or subsequent to the administering the lymphoid cell. In some embodiments, of a method described herein, the administering a compound described herein, is performed separately from the administering the lymphoid cell. In some embodiments, a cell of the subject typically exhibits expression or activity of PTPN2

In an aspect, provided is a method of potentiating anti-tumor or anti-cancer immunity of a subject in need thereof, comprising: (a) contacting a lymphoid cell of the subject with a compound described herein, thereby potentiating the anti-tumor or anti-cancer immunity of the subject. In some embodiment, contemplated immunity comprises anti-tumor, anti-cancer activity, anti-viral infection activity, and/or anti-bacterial infection activity. In yet another aspect, provided is a method of treating tumor or cancer of a subject in need thereof, comprising: (a) contacting a lymphoid cell of the subject with a compound described herein, thereby treating the tumor or cancer of the subject. In some embodiments, the step of contacting a lymphoid cell is performed in vivo. In some embodiments, the step of contacting is performed ex vivo, and subsequently followed by introducing the lymphoid cell to the subject.

In some embodiments, the lymphoid cell being contacted by a compound disclosed herein comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiment, accordingly, the method further comprises (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments of a method described herein, (a) is performed prior to, concurrent with, or subsequent to (b). In an aspect, provided is a method of potentiating anti-tumor or anti-cancer immunity of a subject in need thereof, comprising: (a) downregulating expression or activity of PTPN2 in a lymphoid cell of the subject, thereby potentiating the anti-tumor or anti-cancer immunity of the subject. In yet another aspect, provided is a method of treating tumor or cancer of a subject in need thereof, comprising: (a) downregulating expression or activity of PTPN2 in a lymphoid cell of the subject, thereby treating the tumor or cancer of the subject. In some embodiments of practicing one or more of these methods, the downregulating is performed in vivo. In some embodiments, the downregulating is performed ex vivo, and subsequently followed by introducing the lymphoid cell to the subject. In some embodiments, the method further comprises administering the lymphoid cell to the subject prior to, concurrent with, or subsequent to the downregulating. In some embodiments, the downregulating comprises introducing a compound described herein, to the lymphoid cell. In some embodiments, the downregulating comprises transiently downregulating the expression or activity of PTPN2. Where desired, the transiently downregulating is performed once or multiple times such as two, three or more times. In some embodiments, a first intermittent dosing regimen of a compound described herein, is the same as that of a second intermittent dosing regimen of the compound. In some embodiments, the first intermittent dosing regimen of a compound described herein is different from that of the second intermittent dosing regimen of the compound. In some embodiments, the method fiurther comprises (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments of a method described herein, (a) is performed prior to, concurrent with, or subsequent to (b).

In an aspect, provided is a method of increasing efficacy or reducing side effect of a cell therapy for a subject in need thereof, comprising: (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and (b) administering a compound described herein to the subject prior to, concurrent with, or subsequent to (a). In some embodiments, the cell including but not limited to lymphoid cell retains expression or activity of PTPN2 prior to (b). In some embodiments a cell of the subject typically exhibits expression or activity of PTPN2.

In an aspect, provided is a method of increasing efficacy or reducing side effect of a cell therapy for a subject in need thereof, comprising: (a) administering to the subject a sub-therapeutic amount of a cell comprising a chimeric antigen receptor (CAR) sequence encoding a CAR, (b) administering a compound described herein, to the subject prior to, concurrent with, or subsequent to (a). In some embodiments, the cell including but not limited to lymphoid cell retains expression or activity of PTPN2 prior to (b).

In some embodiments, the compounds disclosed herein or utilized in the method disclosed reduces PTPN2 signaling in a cell. In some embodiments, the compounds disclosed herein do not regulate site-specific recombination of a gene encoding PTPN2. In some embodiments, the compounds disclosed herein are configured to bind PTPN2, or are exhibiting binding specificity to PTPN2 in comparison to other tyrosine phosphatases. In some embodiments, a compound described herein exhibits IC₅₀ of less than or equal to 5 μM for PTPN2.

In some embodiments, the practice of any of the subject methods further comprises monitoring, concurrent with or subsequent to the administration of the compound described herein, and/or the lymphoid cell, one or more health parameters of the subject selected from the group consisting of: temperature, wheezing, sweating, fatigue, weight, insomnia, diarrhea, infections, and mental disorders. In some embodiments, the method further comprises detecting, concurrent with or subsequent to the administration of the compound described herein, the lymphoid cell, one or more inflammatory biomarkers selected from the group consisting of: antibodies, cytokines, radicals, and coagulation factors, disclosed herein. In some embodiments, practicing a subject method may involve (1) contacting a cell with a compound described herein (2) administering a lymphoid cell to the subject, (3) downregulating the expression or activity of PTPN2 in a cell of a subject, (4) administering a compound described herein, to a subject, (5) contacting a lymphoid cell of a subject with a compound described herein, and/or (6) downregulating the expression or activity of PTPN2 in the lymphoid cell of the subject is performed prior to, concurrent with, or subsequent to an administration of another agent (second agent) or therapy to the subject. In some embodiments, the second agent is selected from the group consisting of a chemotherapeutic agent, a radioactive agent, a small molecule agent targeting a tumor marker, an antigen-binding agent specifically binding to a tumor marker, and an immune modulator. In some embodiments, the second agent is a checkpoint inhibitor. In some embodiments, the second agent includes without limitation an inhibitor of PD1, PD-L1, LAG3, CTLA4, CD160, BTLA, LAIR1, TIM3, 2B4, CD93, OX40, Siglec-15, and TIGIT. In some embodiments, the second agent is an inhibitor of IDO or mTOR. In some embodiments, a second therapy to be combined is a cell therapy comprising stem cells or lymphoid cells.

In some embodiments of a method described herein, the TFP comprises a TCR subunit that comprises (1) a TCR extracellular domain capable of specific binding to an antigen, and (2) an intracellular signaling domain, wherein the TFP forms a TCR complex. In some embodiments, the TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together. In some embodiments, the TCR intracellular domain comprises a stimulatory domain from an intracellular signaling domain of epsilon chain, delta chain, and/or a gamma chain of cluster of differentiation 3 (CD3). In some embodiments, the TCR intracellular domain comprises a stimulatory domain from an intracellular signaling domain of TCR alpha, or from an intracellular signaling domain of TCR beta. In some embodiments, the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154. In some embodiments, the TFP comprises a costimulatory domain. In some embodiments, the costimulatory domain of the TFP is selected from the group consisting of: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments of a method described herein, the CAR comprises an antigen-binding domain and an intracellular signaling domain. In some embodiments, the intracellular signaling domain of the CAR comprises a primary signaling domain and/or a costimulatory signaling domain, wherein the primary signaling domain comprises a functional signaling domain of a protein chosen from CD3 zeta, CD3 gamma, CD3 delta, CD3 epsilon, common FcR gamma (FCERIG), FcR beta (Fc Epsilon Rib), CD79a, CD79b, Fcgamma Rlla, DAP10, or DAP12. In some embodiments, the intracellular signaling domain of the CAR comprises a costimulatory signaling domain that comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In some embodiments, the intracellular signaling domain of the CAR comprises a primary signaling domain and/or a costimulatory signaling domain, wherein the primary signaling domain and/or the costimulatory signaling domain is minimally required for activation of the CAR upon binding to an antigen. In some embodiments, the CAR is a first generation CAR in which the primary signaling domain is a member selected from the group consisting of CD3zeta, CD28, 4-1BB, OX40, DAP10, ICOS, and a variant thereof. In some embodiments, the CAR is a second generation CAR in which (i) the primary signaling domain is a member selected from the group consisting of CD3zeta, CD28, 4-1BB, OX40, DAP10, ICOS, and a variant thereof, and (ii) the co-stimulatory signaling domain is a different member selected from the group consisting of CD3zeta, CD28, 4-1BB, OX40, DAP10, ICOS, and a variant thereof. In some embodiments of a method described herein, the antigen is a tumor antigen or cancer antigen a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-llRa, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTl, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments of a method described herein, the antigen comprises a neoantigen encoded by a tumor-specific mutated gene. In some embodiments of a method described herein, the side effect comprises cytokine release syndrome (CRS), inflammatory disorder, or autoimmune disorder.

In an aspect, provided is a modified cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, wherein expression or activity of PTPN2 in the cell is downregulated, to potentiate immunity of the modified cell. In some embodiments, the modified cell exhibits a mutation of (i) a first gene encoding PTPN2 or (ii) a second gene operatively linked to PTPN2, wherein the mutation inhibits the expression and/or activity of PTPN2. In some embodiments, the expression or activity of PTPN2 is transiently downregulated in the modified cell. In some embodiments of the modified cell, the expression or activity of PTPN2 is downregulated by a compound described herein. In some embodiments, a subject compound modifying the cell (i) does not regulate site-specific recombination of a gene encoding PTPN2.; (ii) is configured to bind PTPN2; and/or (iii) exhibits binding specificity to PTPN2 in comparison to other tyrosine phosphatases. In some embodiments, a subject compound modifying a cell is a the small molecule exhibiting IC₅₀ of less than or equal to 5 μM for PTPN2. In some embodiments, the TFP (contained in a modified cell) comprises a TCR subunit that comprises (1) a TCR extracellular domain capable of specific binding to the antigen, and (2) an intracellular signaling domain, wherein the TFP forms a TCR complex. In some embodiments, the TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together. In some embodiments, the TCR intracellular domain comprises a stimulatory domain from an intracellular signaling domain of epsilon chain, delta chain, and/or a gamma chain of cluster of differentiation 3 (CD3). In some embodiments, the TCR intracellular domain comprises a stimulatory domain from an intracellular signaling domain of TCR alpha, or from an intracellular signaling domain of TCR beta. In some embodiments, the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154. In some embodiments, the TFP comprises a costimulatory domain, including without limitation: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In some embodiments, the modified cell is a modified lymphoid cell. In some embodiments, the modified lymphoid cell is a member or variant thereof selected from the group consisting of: a T cell, B cell, NK cell, KHYG cell, T helper cell, regulatory T cell, memory T cell, tumor infiltration T cell (TIL), antigen presenting cell, and dendritic cell. In some embodiments of the modified cell, the modified lymphoid cell is a variant of a member selected from the group consisting of: a CD4+ T cell, a CD8+ T cell, and a CD4+ and CD8+ T cell.

In some embodiments, any of the subject compounds, modified cells, and any other agent described herein, may be applied (e.g., utilized) in any of the subject methods described herein. Similarly, any compounds, modified cell and any other agent described in the context of a subject method is embodied and contemplated as a subject composition.

### BRIEF DESCRIPTION OF DRAWINGS

FIG. 1A-1C. FIG. 1A. MHC1 Assay for PTPN2 Cellular Activity. Clusters of points represent, from left to right in form of a group of 3 vertical dots representative of replicate experiments: 1 ng/ml IFNγ DMSO, a first reference compound at 100 μM, the first reference compound at 50 μM, a subject compound in Table 6, a second reference compound, a third reference compound, another subject compound in Table 6, and yet another subject compound in Table 6, a fourth reference compound; FIG. 1B. Clusters of points represent, from left to right: No IFNγ, IFNγ, the fourth reference compound at 10 μM, a subject compound in Table 6 at 10 μM; Y-axis % MHCI Increase (Normalized) 0-200; FIG. 1C. Clusters of points represent, from left to right: No IFNγ, IFNγ, the fourth reference compound at 10 μM, a subject compound in Table 6 at 10 μM, another subject compound in Table 6 at 10 μM, and yet another subject compound in Table 6 at 10 μM; Y-axis % MHCI Increase (Normalized) 0-200. The results demonstrate that subject compounds exhibiting PTPN2 inhibitory activity enhance MHC expression level.

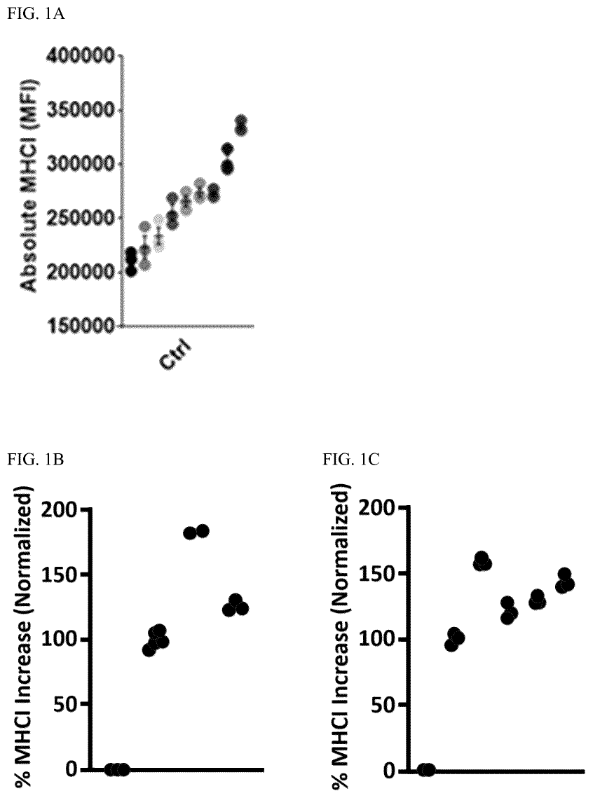

FIG. 2 depicts tumor infiltrating lymphocyte exhaustion as shown by high TIM3 and PD1 expression and low Slamf6 expression in tumor associated CD8+ cells but not spleen associated cells.

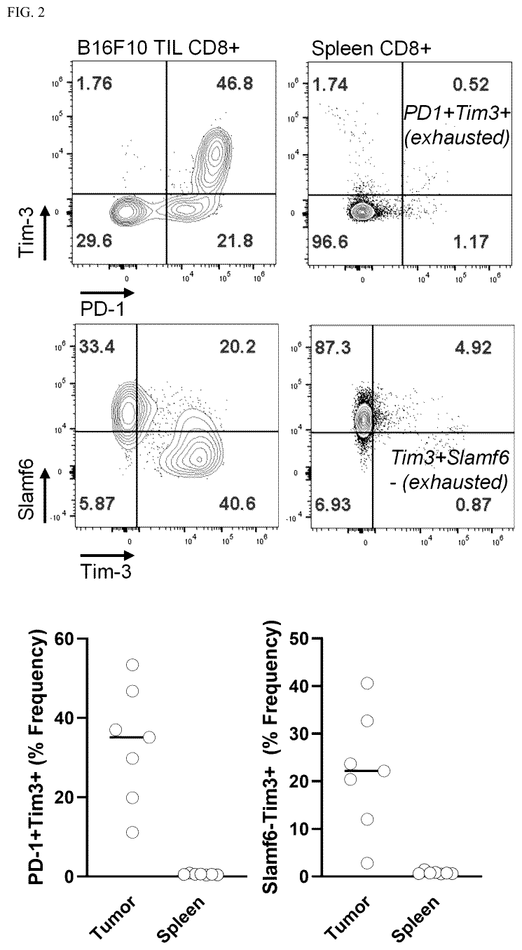

FIG. 3 depicts tumor growth inhibition using B16F10 mouse model treated with a subject PTPN2 inhibitor disclosed herein.

FIG. 4A depicts day 7 tumor infiltrating CD8 T cell phenotype in B16F10 Model, and FIG. 4B depicts day 7 tumor infiltrating macrophage phenotype in B16F10 Model.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

“About” as used herein when referring to a measurable value such as an amount, a duration, and the like, is meant to encompass variations of ±10% of a stated number or value.

As used herein, “expression” refers to the process by which a polynucleotide is transcribed into mRNA and/or the process by which the transcribed mRNA (also referred to as a “transcript”) is subsequently translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell. The level of expression (or alternatively, the “expression level”) of a PTPN2 gene can be determined, for example, by determining the level of PTPN2polynucleotides, polypeptides or gene products.

“Aberrantly expressed” or “aberrant expression” as applied to a nucleotide sequence (e.g., a gene) or polypeptide sequence in a subject, refers to the aberrant production of the mRNA transcribed and/or translated from the nucleotide sequence or the protein product encoded by the nucleotide sequence. A differentially expressed sequence may be overexpressed (or aberrantly high expression) or underexpressed (or aberrantly low expression) as compared to the expression level of a reference sample (i.e., a reference level). As used herein, overexpression is an increase in expression can be at least 1.25 fold, or alternatively, at least 1 fold, or alternatively, at least 2 fold, or alternatively, at least 3 fold, or alternatively, at least 4 fold, or alternatively, at least 10 fold expression over that detected in a reference sample. As used herein, underexpression is a reduction in expression can be at least 1.25 fold, or alternatively, at least 1 fold, or alternatively, at least 2 fold, or alternatively, at least 3 fold, or alternatively, at least 4 fold, or alternatively, at least 10 fold expression under that detected in a reference sample. Underexpression also encompasses absence of expression of a particular sequence as evidenced by the absence of detectable expression in a test subject when compared to a reference sample.

“Signal transduction” is a process during which stimulatory or inhibitory signals are transmitted into and within a cell to elicit an intracellular response. A molecule can mediate its signaling effect via direct or indirect interaction with downstream molecules of the same pathway or related pathway(s). For instance, PTPN2 signaling can involve a host of downstream molecules including but not limited to one or more of the following proteins: STAT1 and STAT5.

A “control” or “control sample” is an alternative sample or subject used in an experiment for comparison purpose.

The term “reference level” refers to a control level used to evaluate a test level. In some examples, a reference level may be a control. For example, a biomarker may be considered to be underexpressed when the expression level of that biomarker is lower than a reference level. The reference level can be determined by a plurality of methods, provided that the resulting reference level accurately provides a level of a biomarker above which exists a first group of subjects having a different probability of exhibiting a clinically beneficial response to treatment with a PTPN2 inhibitor than that of a second group of patients having levels of the biomarker below the reference level. The reference level may be determined, for example, by measuring the level of expression of a biomarker in tumorous or non-tumorous cancer cells from the same tissue as the tissue of the cancer cells to be tested. In some examples, the reference level may be a level of a biomarker determined in vitro. A reference level may be determined by comparison of the level of a biomarker in populations of subjects having the same cancer. Two or more separate groups of subjects may be determined by identification of subsets of populations of the cohort that have the same or similar levels of a biomarker. Determination of a reference level can then be made based on a level that distinguishes these separate groups. A reference level may be a single number, equally applicable to every subject, or a reference level can vary according to specific subpopulations of subjects. For example, older men may have a different reference level than younger men for the same cancer, and women may have a different reference level than men for the same cancer. Furthermore, the reference level may be some level determined for each subject individually. For example, the reference level may be a ratio of a biomarker level in a cancer cell of a subject relative to the biomarker level in a normal cell within the same subject. In some embodiments, a reference level is a numerical range of gene expression that is obtained from a statistical sampling from a population of individuals having cancer. The sensitivity of the individuals having cancer to treatment with a PTPN2 inhibitor may be known. In some embodiments, the reference level is derived by comparing gene expression to a control gene that is expressed in the same cellular environment at relatively stable levels (e.g. a housekeeping gene such as an actin). Comparison to a reference level may be a qualitative assessment or a quantitative determination.

The terms “determining,” “measuring,” “evaluating,” “assessing,” “assaying,” “testing,” and “analyzing” are used interchangeably herein to refer to any form of measurement, and include determining if an analyte is present or not (e.g., detection). These terms can include both quantitative and/or qualitative determinations. Assessing may be relative or absolute. A relative amount could be, for example, high, medium or low. An absolute amount could reflect the measured strength of a signal or the translation of this signal strength into another quantitative format, such as micrograms/mL. “Detecting the presence of” can include determining the amount of something present, as well as determining whether it is present or absent.

The terms “antagonist” and “inhibitor” are used interchangeably, and they refer to a compound, or a biological molecule having the ability to effect inhibition of a biological function of a target protein (e.g., PTPN2), whether by inhibiting the activity or expression of the target protein. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. Alternatively or in addition to, an activity of a target protein may involve interaction (e.g., binding) between the target protein and a substrate of the target protein, and the terms “antagonist” and “inhibitors” can refer to a compound having the ability to interact with (e.g., bind to) the subject of the target protein, to indirectly inhibit the biological activity of the target protein. In some cases, such compound may bind both the target protein and one or more kinds of the substrate. A preferred biological activity inhibited by an antagonist is associated with the development, growth, maintenance, or spread of a cancer or a tumor.

The term “cell proliferation” refers to a phenomenon by which the cell number has changed as a result of division. This term also encompasses cell growth by which the cell morphology has changed (e.g., increased in size) consistent with a proliferative signal.

The terms “administer,” “administering,” “administration,” and derivatives thereof refer to the methods that may be used to enable delivery of agents or compositions to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease.

The term “systemic administration” refers to administration of agents or compositions such that the agents or compositions become distributed in a subject's body. The distribution of the agents or compositions throughout the subject's body may be an even distribution. Alternatively, the distribution may be preferential, resulting in a higher localization of the agents or compositions in one or more desired sites. A desired site may be the blood or another site that is reachable by the vascular system. Non-limiting examples of systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood. By contrast, “non-systemic administration” refers to administration of agents or compositions such that the agents or compositions are administered locally to the target site of interest of a subject's body to effect primarily a local effect.

The terms “co-administration,” “administered in combination with,” and their grammatical equivalents, encompass administration of two or more agents to a subject so that both agents and/or their metabolites can assert their respective functions. Co-administration includes simultaneous administration in separate compositions, administration at different times in separate compositions, or administration in a composition in which both agents are present.

The term “subject” includes, but is not limited to, humans of any age group, e.g., a pediatric subject (e.g., infant, child or adolescent) or adult subject (e.g., young adult, middle-aged adult or senior adult)) and/or other primates (e.g., cynomolgus monkeys or rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The methods described herein can be useful in both human therapeutics and veterinary applications. In some embodiments, the subject is a mammal, and in some embodiments, the subject is human.

The term “downregulating PTPN2 activity”, as used herein, refers to slowing, reducing, altering, inhibiting, as well as completely eliminating and/or preventing PTPN2 activity.

The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function.

The terms “immune effector cell” and “effector cell” are used interchangeably here. They refer to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

The term “lymphoid cell” or “lymphoid cells” refers to any of the cells responsible for the production of immunity mediated by cells or antibodies and including lymphocytes, lymphoblasts, and plasma cells. Lymphoid cells include granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells (including activated T cells). In some examples, T cells include both naive and memory cells (e.g. central memory or T_{CM}, effector memory or T_{EM} and effector memory RA or T_{EMRA}), effector cells (e.g. cytotoxic T cells or CTLs or Tc cells), helper cells (e.g. Th1, Th2, Th3, Th9, Th7, TFH), regulatory cells (e.g. Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage.

The term “transiently downregulated” as used herein generally means that a downregulation of expression or activity of a target molecule (e.g., PTPN2) is not permanent. A transient downregulation may not be a permanent downregulation. In some cases, a transient downregulation may involve downregulating (e.g., reducing) expression or activity of a target molecule for a period of time, followed by regaining at least a portion of expression or activity level of the target molecule that was previously downregulated. A transient downregulation can involve an intermittent downregulation of a target molecule (e.g., PTPN2).

The term “intermittent” is used herein to describe a process that is not continuous. An intermittent process may be followed by a break or stop. A plurality of intermittent processes may involve alternatively starting and stopping a same process or different processes. In some embodiments, the term “intermittent dosing regimen” as used here refers to a dosing regimen that comprises administering a pharmaceutical composition, followed by a rest period.

The term “side effect” as used herein refers to any complication, unwanted, or pathological outcome of a therapy that occurs in addition to or in place of a desired treatment outcome of the therapy. Examples of a side effect may include, but are not limited to, (i) off-target cell toxicity, (ii) on-target off-tumor toxicity, and/or (iii) autoimmunity (e.g., chronic autoimmunity).

The term “efficacy” of a treatment or method, as used herein, can be measured based on changes in the course of disease or condition in response to such treatment or method. For example, the efficacy of a treatment or method of the present disclosure may be measured by its impact on signs or symptoms of a disease or condition of a subject, e.g., a tumor or cancer of the subject. A response may be achieved when a subject having the disease or condition experiences partial or total alleviation of the disease or condition, or reduction of one or more symptoms of the disease or condition. In an example, a response is achieved when a subject suffering from a tumor exhibits a reduction in the tumor size after the treatment or method, as provided in the present disclosure. In some examples, the efficacy may be measured by assessing cancer cell death, reduction of tumor (e.g., as evidenced by tumor size reduction), and/or inhibition of tumor growth, progression, and dissemination.
“Amino” refers to the —NH2 radical.
“Cyano” refers to the —CN radical.
“Nitro” refers to the —NO2 radical.
“Oxa” refers to the —O— radical.
“Oxo” refers to the ═O radical.
“Thioxo” refers to the ═S radical.
“Imino” refers to the ═N—H radical.
“Oximo” refers to the ═N—OH radical.
“Hydrazino” refers to the ═N—NH₂ radical.

The term “moiety” refers to a specific segment or functional group of a molecule. Chemical moieties are often recognized chemical entities embedded in or appended to a molecule.

“Optional” or “optionally” means that a subsequently described event or circumstance may or may not occur and that the description includes instances when the event or circumstance occurs and instances in which it does not.

The term “optionally substituted” or “substituted” means that the referenced group may be substituted with one or more additional group(s) individually and independently selected from alkyl, alkenyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R³ is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂(where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” or “alkoxyl” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In some embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂(where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In some embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The term “aromatic” refers to a planar ring having a delocalized t-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

The term “cycloalkyl” refers to a monocyclic or polycyclic non-aromatic radical, wherein each of the atoms forming the ring (i.e. skeletal atoms) is a carbon atom. Cycloalkyls may be saturated or partially unsaturated. In some embodiments, a cycloalkyl ring is fused with an aryl, heteroaryl, heterocycloalkyl, or a second cycloalkyl ring. In some embodiments, a cycloalkyl ring is a spirocyclic cycloalkyl ring. In some embodiments, cycloalkyl groups include groups having from 3 to 10 ring atoms. Depending on the structure, a cycloalkyl group can be a monoradical or a diradical (i.e., a cycloalkylene group).

In some embodiments, “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. In some embodiments, the cycloalkyl is attached to the rest of the molecule by a single bond. Cycloalkyl is saturated (i.e., containing single C—C bonds only) or unsaturated (i.e., containing one or more double bonds or triple bonds). Examples of monocyclic cycloalkyls include, e.g., cyclopropyl, cyclobutyl, cyclopentyl, cyclohexyl, cycloheptyl, and cyclooctyl. An unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkylalkyl” refers to a radical of the formula —R^{c}-cycloalkyl where R^{c} is an alkylene chain as defined above. The alkylene chain and the cycloalkyl radical may be optionally substituted.

“Cycloalkylalkynyl” refers to a radical of the formula —R^{c}-cycloalkyl where R^{c} is an alkynylene chain as defined above. The alkynylene chain and the cycloalkyl radical may be optionally substituted as defined above.

“Cycloalkylalkoxy” refers to a radical bonded through an oxygen atom of the formula —O—R^{c}-cycloalkyl where R^{c} is an alkylene chain as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

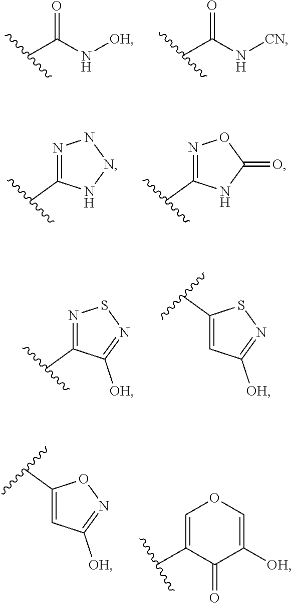

and the like.

“Halo” or “halogen” refers to bromo, chloro, fluoro or iodo substituents.

The term “haloalkyl” refers to an alkyl group that is substituted with one or more halogens. The halogens may be the same or they may be different. Non-limiting examples of haloalkyls include —CH₂Cl, —CF₃, —CHF₂, —CH₂CF₃, —CF₂CF₃, and the like.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “bond” or “single bond” refers to a chemical bond between two atoms, or two moieties when the atoms joined by the bond are considered to be part of larger substructure.

As used herein, unless otherwise stated specifically, the substituent “R” appearing by itself and without a number designation refers to a substituent selected from among from alkyl, haloalkyl, heteroalkyl, alkenyl, alkynyl, cycloalkyl, aryl, heteroaryl (bonded through a ring carbon), and heterocycloalkyl.

A “heterocycloalkyl” group or “heteroalicyclic” group refers to a cycloalkyl group, wherein at least one skeletal ring atom is a heteroatom selected from nitrogen, oxygen and sulfur. Heterocycloalkyls may be saturated or partially unsaturated. In some embodiments, a heterocycloalkyl ring is fused with an aryl, heteroaryl, cycloalkyl, or a second heterocycloalkyl ring. The term heterocycloalkyl also includes all ring forms of the carbohydrates, including but not limited to the monosaccharides, the disaccharides and the oligosaccharides. In some embodiments, a heterocycloalkyl ring is a spirocyclic heterocycloalkyl ring. In some embodiments, a heterocycloalkyl ring is a bridged heterocycloalkyl ring. Unless otherwise noted, heterocycloalkyls may have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Depending on the structure, a heterocycloalkyl group can be a monoradical or a diradical (i.e., a heterocycloalkylene group).

In some embodiments, “heterocycloalkyl” refers to a stable 3- to 18-membered non-aromatic ring radical that comprises two to twelve carbon atoms and from one to six heteroatoms selected from nitrogen, oxygen and sulfur. In some embodiments, unless stated otherwise specifically in the specification, the heterocycloalkyl radical is a monocyclic, bicyclic, tricyclic or tetracyclic ring system, which optionally includes fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofiuryl, trithianyl, tetrahydropyranyl, thiomorpholinyl, thiamorpholinyl, 1-oxo-thiomorpholinyl, and 1,1-dioxo-thiomorpholinyl.

“N-heterocycloalkyl” or “N-attached heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. Examples of such N-heterocycloalkyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocycloalkyl” or “C-attached heterocycloalkyl” refers to a heterocycloalkyl radical as defined above containing at least one heteroatom and where the point of attachment of the heterocycloalkyl radical to the rest of the molecule is through a carbon atom in the heterocycloalkyl radical. Examples of such C-heterocycloalkyl radicals include, but are not limited to, 2-morpholinyl, 2- or 3- or 4-piperidinyl, 2-piperazinyl, 2- or 3-pyrrolidinyl, and the like.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical may be optionally substituted. The heterocycloalkyl part of the heterocycloalkylalkyl radical may be optionally substituted.

“Heterocycloalkylalkoxy” refers to a radical bonded through an oxygen atom of the formula —O—R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkoxy radical may be optionally substituted. The heterocycloalkyl part of the heterocycloalkylalkoxy radical may be optionally substituted.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). In some embodiments, unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

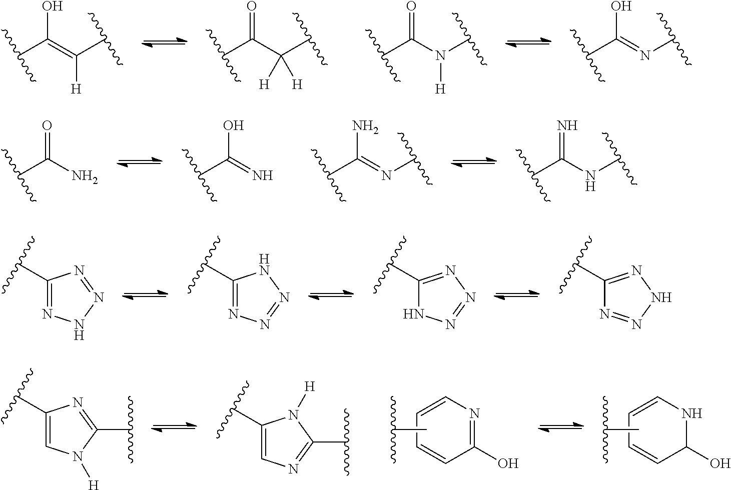

In some instances, the compounds disclosed herein exist in tautomeric forms. The structures of said compounds are illustrated in the one tautomeric form for clarity. The alternative tautomeric forms are expressly included in this disclosure, such as, for example, the structures illustrated below.

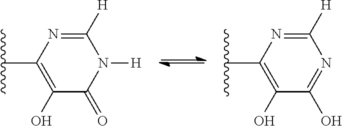 [SMILES: *c1nc[nH]c(=O)c1O ; *c1ncnc(O)c1O]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

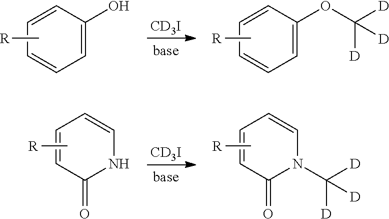

Deuterium-transfer reagents, such as lithium aluminum deuteride (LiAID₄), are employed to transfer deuterium under reducing conditions to the reaction substrate. The use of LiAID₄ is illustrated, by way of example only, in the reaction schemes below.

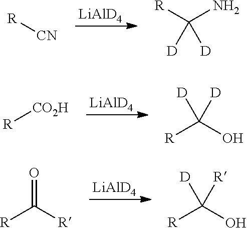

Deuterium gas and palladium catalyst are employed to reduce unsaturated carbon-carbon linkages and to perform a reductive substitution of aryl carbon-halogen bonds as illustrated, by way of example only, in the reaction schemes below.

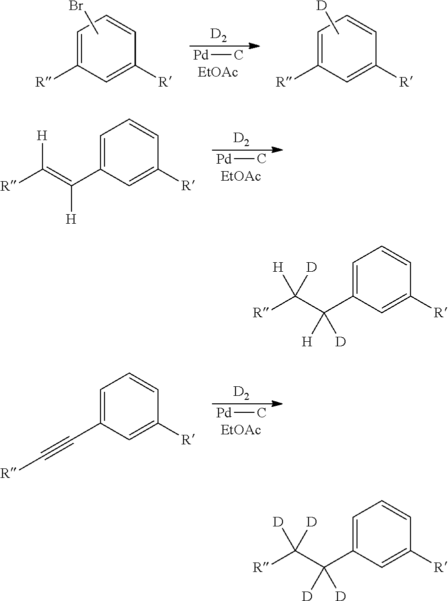

“Pharmaceutically acceptable salt” includes both acid and base addition salts. A pharmaceutically acceptable salt of any of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject). In some embodiments, “treatment”, “treating”, palliating”, or “ameliorating” does not include a prophylactic benefit.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

### Compounds

The compounds, or a pharmaceutically acceptable salts or solvates thereof, disclosed herein are PTPN2 inhibitors and have a wide range of applications in therapeutics, diagnostics, and other biomedical research.

In as aspect is provided a compound of Formula (I), or a pharmaceutically acceptable salt or solvate thereof:

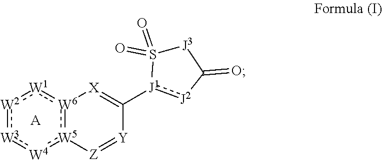

wherein:
W¹ is N, N(R¹), O, C(R), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴).
W⁵ is N or C;
W⁶ is N or C; wherein at least one of W¹, W², W³, W⁴, W⁵, and W⁶ is N, N(R¹), N(R²), N(R³), N(R⁴), or O; and
X is N or C(R⁵);
Y is N or C(R⁶);
Z is N or C(R¹)
J¹ is N, C, or C(R¹);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
R^{12a} is selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20f};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and
provided that:
i) at least two of Ring A [symbol] are double bonds;
ii) when J³ is CH₂; R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}; and
iii) the compound is not

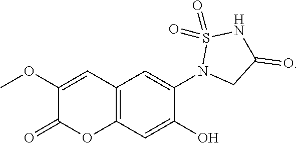 [SMILES: COc1cc2cc(N3CC(=O)NS3(=O)=O)c(O)cc2oc1=O]

It will be understood that the formulae below, for example, are equivalent resonance structure representations of one compound formula wherein at least two of Ring A [symbol] are double bonds:

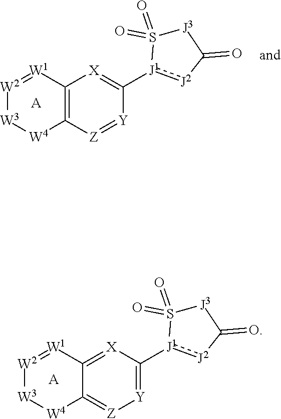

In some embodiments, when J³ is C(R¹⁰)(R^{10a}); R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}.

In some embodiments, W¹ is N, N(R¹), O, C(R¹), C(R¹)(R^{1a}), C(O), S(O), S(O)₂, S(O)(NR¹), S(O)(R¹), or P(O)(R¹); W² is N, N(R²), O, C(R²), C(R²)(R^{2a}), C(O), S(O), S(O)₂, S(O)(NR²), S(O)(R²), or P(O)(R²); W³ is N, N(R³), O, C(R³), C(R³)(R^{3a}), C(O), S(O), S(O)₂, S(O)(NR³), S(O)(R³), or P(O)(R³); W⁴ is N, N(R⁴), C(R⁴), C(R⁴)(R^{a}), C(O), S(O), S(O)₂, S(O)(NR⁴), S(O)(R⁴), or P(O)(R⁴). W⁵ is N or C; W⁶ is N or C wherein at least one of W¹, W², W³, W⁴, W⁵, and W⁶ is N, N(R¹), N(R²), N(R³), N(R⁴), or O; and at least two of Ring A [symbol] are double bonds.

In some embodiments, W¹ is N, N(R¹), O, C(R¹), C(R¹)(R^{1a}), C(O), S(O), S(O)₂, S(O)(NR¹), S(O)(R¹), or P(O)(R¹); W² is N, N(R²), O, C(R²), C(R²)(R^{2a}), C(O), S(O), S(O)₂, S(O)(NR²), S(O)(R²), or P(O)(R²); W³ is N, N(R³), O, C(R³), C(R³)(R^{3a}), S(O), S(O)₂, S(O)(NR³), S(O)(R³), or P(O)(R³); W⁴ is N, N(R⁴), C(R⁴), C(R⁴)(R^{a}), C(O), S(O), S(O)₂, S(O)(NR⁴), S(O)(R⁴), or P(O)(R⁴); W⁵ is N or C; W⁶ is N or C; wherein at least one of W¹, W², W³, W⁴, W⁵, and W⁶ is N, N(R¹), N(R²), N(R³), N(R⁴), or O; and at least two of Ring A [symbol] are double bonds.

In some embodiments, the compound is a compound of Formula (Ia):

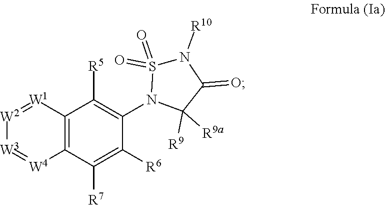

wherein at least one of W¹, W², W³, and W⁴ is N. R⁵, R⁶, R⁷, R⁹, R^{9a}, and R¹⁰ are as described herein.

In some embodiments, the compound is a compound of Formula (Ib):

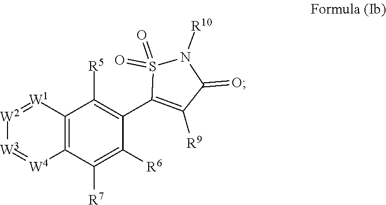

wherein at least one of W¹, W², W³, and W⁴ is N. R⁵, R⁶, R⁷, R⁹, and R¹⁰ are as described herein.

In some embodiments, the compound is a compound of Formula (Ic):

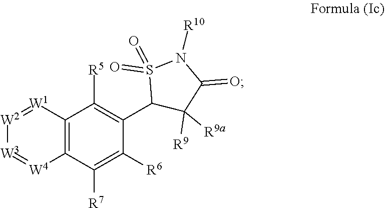

wherein at least one of W¹, W², W³, and W⁴ is N. R⁵, R⁶, R⁷, R⁹, R^{9a}, and R¹⁰ are as described herein.

In some embodiments, the compound is a compound of Formula (Ig):

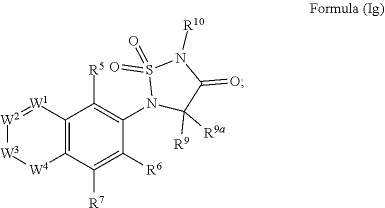

wherein at least one of W¹, W², W³, and W⁴ is N, N(R³), N(R⁴), or O. R⁵, R⁶, R⁷, R⁹, R^{9a}, and R¹⁰ are as described herein.

In some embodiments, the compound is a compound of Formula (Ih):

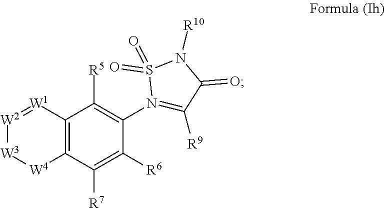

wherein at least one of W¹, W², W³, and W⁴ is N, N(R³), N(R⁴), or O. R⁵, R⁶, R⁷, R⁹, and R¹⁰ are as described herein.

In some embodiments, the compound is a compound of Formula (Ii):

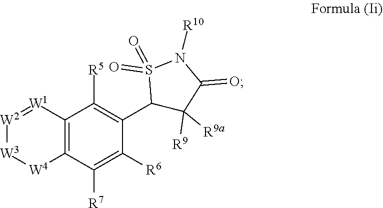

wherein at least one of W¹, W², W³, and W⁴ is N, N(R³), N(R⁴), or O. R⁵, R⁶, R⁷, R⁹, R^{9a}, and R¹⁰ are as described herein.

In some embodiments, the compound is a compound of Formula (Ij):

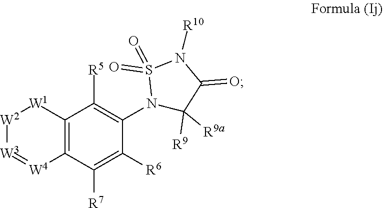

wherein at least one of W¹, W², W³, and W⁴ is N(R¹), N(R²), N, or O. R⁵, R⁶, R⁷, R⁹, R^{9a}, and R¹⁰ are as described herein.

In some embodiments, the compound is a compound of Formula (Ik):

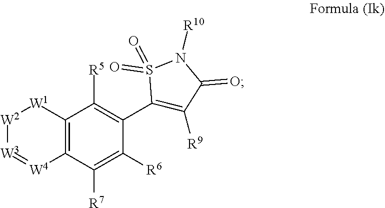

wherein at least one of W¹, W², W³, and W⁴ is N(R¹), N(R²), N, or O. R⁵, R⁶, R⁷, R⁹, and R¹⁰ are as described herein.

In some embodiments, the compound is a compound of Formula (Im):

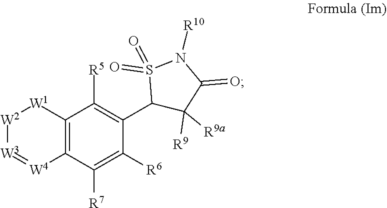

wherein at least one of W¹, W², W³, and W⁴ is N, N(R³), N(R⁴), or O. R⁵, R⁶, R⁷, R⁹, R^{9a}, and R¹⁰ are as described herein.

In some embodiments, the compound is a compound of Formula (Iv):

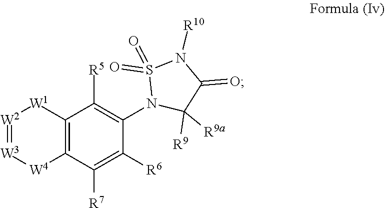

wherein at least one of W¹, W², W³, and W⁴ is N(R¹), N(R⁴), N, or O. R⁵, R⁶, R⁷, R⁹, R^{9a}, and R¹⁰ are as described herein.

In some embodiments, the compound is a compound of Formula (Iw):

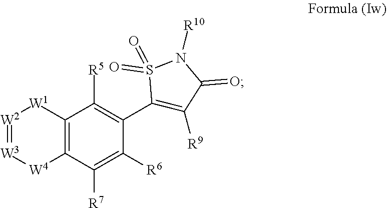

wherein at least one of W¹, W², W³, and W⁴ is N(R¹), N(R⁴), N, or O. R⁵, R⁶, R⁷, R⁹, and R¹⁰ are as described herein.

In some embodiments, the compound is a compound of Formula (Ix):

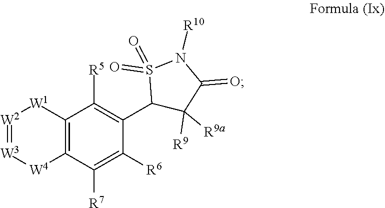

wherein at least one of W¹, W², W³, and W⁴ is N(R¹), N(R⁴), N, or O. R⁵, R⁶, R⁷, R⁹, R^{9a}, and R¹⁰ are as described herein.

In some embodiments, the compound is a compound of Formula (Io):

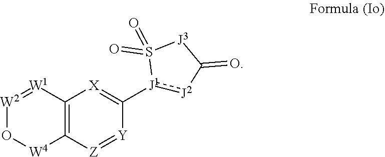

W¹, W², W⁴, X, Y, Z, JV, J², and J³ are as described herein. In some embodiments, W¹ is C(R¹); W² is C(R²); and W⁴ is C(R⁴)(R^{4a}), C(O), S(O), S(O)₂, S(O)(NR⁴), S(O)(R⁴), or P(O)(R⁴).

In some embodiments, the compound is a compound of Formula (Ioa):

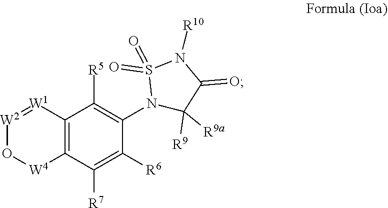

W¹, W², W⁴, R⁵, R⁶, R⁷, R⁹, R^{9a}, and R¹⁰ are as described herein. In some embodiments, W¹ is C(R¹); W² is C(R²); and W⁴ is C(R⁴)(R^{4a}), C(O), S(O), S(O)₂, S(O)(NR⁴), S(O)(R⁴), or P(O)(R⁴).

In some embodiments, the compound is a compound of Formula (Iob):

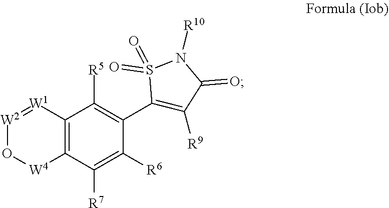

W¹, W², W⁴, R⁵, R⁶, R⁷, R⁹, and R¹⁰ are as described herein. In some embodiments, W¹ is C(R¹); W² is C(R²); and W⁴ is C(R⁴)(R^{4a}), C(O), S(O), S(O)₂, S(O)(NR⁴), S(O)(R⁴), or P(O)(R⁴).

In some embodiments, the compound is a compound of Formula (Ioc):

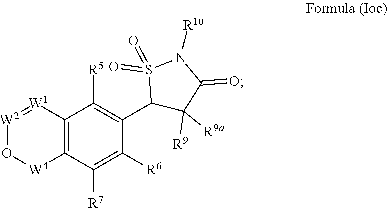

W¹, W², W⁴, R⁵, R⁶, R⁷, R⁹, R^{9a}, and R¹⁰ are as described herein. In some embodiments, W¹ is C(R¹); W² is C(R²); and W⁴ is C(R⁴)(R^{4a}), C(O), S(O), S(O)₂, S(O)(NR⁴), S(O)(R⁴), or P(O)(R⁴).

In some embodiments, the compound is a compound of Formula (Ip):

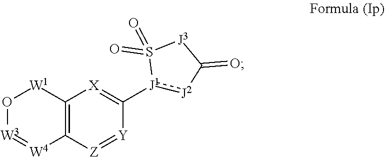

W¹, W³, W⁴, X, Y, Z, JP, J², and J³ are as described herein. In some embodiments, W¹ is C(R¹)(R^{1a}), C(O), S(O), S(O)₂, S(O)(NR¹), S(O)(R¹), or P(O)(R¹); W³ is C(R³); and W⁴ is C(R⁴).

In some embodiments, the compound is a compound of Formula (Ipa):

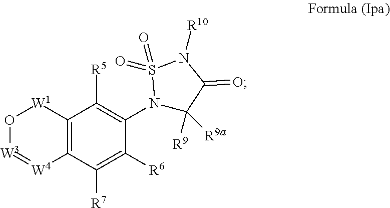

W¹, W³, W⁴, R⁵, R⁶, R⁷, R⁹, R^{9a}, and R¹⁰ are as described herein. In some embodiments, W¹ is C(R¹)(R^{1a}), C(O), S(O), S(O)₂, S(O)(NR¹), S(O)(R¹), or P(O)(R¹); W³ is C(R³); and W⁴ is C(R⁴).

In some embodiments, the compound is a compound of Formula (Ipb):

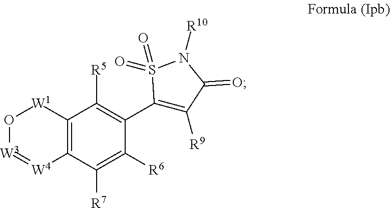

W¹, W³, W⁴, R⁵, R⁶, R⁷, R⁹, and R¹⁰ are as described herein. In some embodiments, W¹ is C(R¹)(R^{1a}), C(O), S(O), S(O)₂, S(O)(NR¹), S(O)(R¹), or P(O)(R¹); W³ is C(R³); and W⁴ is C(R⁴).

In some embodiments, the compound is a compound of Formula (Ipc):

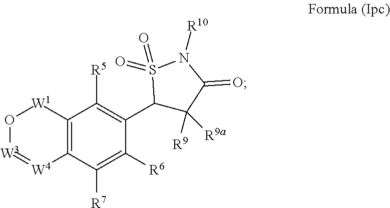

W¹, W³, W⁴, R⁵, R⁶, R⁷, R⁹, R^{9a}, and R¹⁰ are as described herein. In some embodiments, W¹ is C(R¹)(R^{1a}), C(O), S(O), S(O)₂, S(O)(NR¹), S(O)(R¹), or P(O)(R¹); W³ is C(R³); and W⁴ is C(R⁴).

In some embodiments, the compound is a compound of Formula (Ipd):

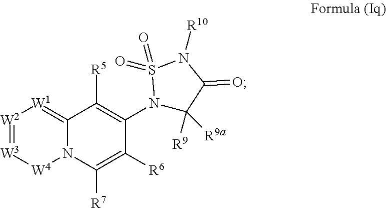

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R⁴), or P(O)(R⁴). R⁵, R⁶, R⁷, R⁹, R^{9a}, and R¹⁰ are as described herein.

In some embodiments, the compound is a compound of Formula (Ir):

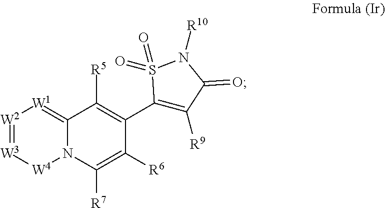

wherein W¹ is N or C(R¹); W² is N or C(R¹); W³ is N or C(R³); and W⁴ is N(R⁴), C(R⁴)(R^{4a}), C(O), S(O), S(O)₂, S(O)(NR⁴), S(O)(R⁴), or P(O)(R⁴). R⁵, R⁶, R⁷, R⁹, and R¹⁰ are as described herein.

In some embodiments, the compound is a compound of Formula (Is):

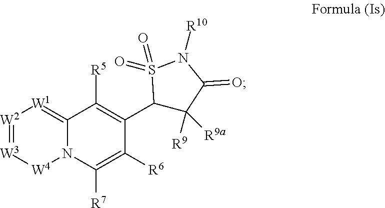

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R), or P(O)(R⁴). R⁵, R⁶, R⁷, R⁹, R^{9a}, and R¹⁰ are as described herein.

In some embodiments, the compound is a compound of Formula (Ipd):

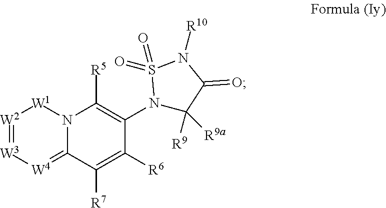

wherein W¹ is N(R¹), C(R¹)(R^{1a}), C(O), S(O), S(O)₂, S(O)(NR¹), S(O)(R), or P(O)(R¹); W² is N or C(R²); W³ is N or C(R³); and
W⁴ is N or C(R⁴). R⁵, R⁶, R⁷, R⁹, R^{9a}, and R¹⁰ are as described herein.

In some embodiments, the compound is a compound of Formula (Ir):

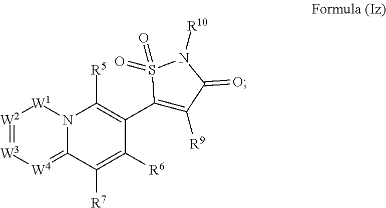

wherein W¹ is N(R¹), C(R¹)(R^{1a}), C(O), S(O), S(O)₂, S(O)(NR¹), S(O)(R), or P(O)(R¹); W² is N or C(R²); W³ is N or C(R³); and
W⁴ is N or C(R⁴). R⁵, R⁶, R⁷, R⁹, and R¹⁰ are as described herein.

In some embodiments, the compound is a compound of Formula (Is):

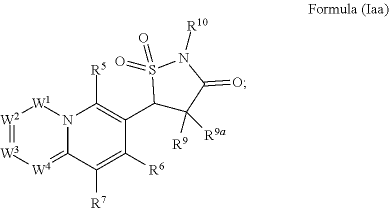

wherein W¹ is N(R¹), C(R¹)(R^{1a}), C(O), S(O), S(O)₂, S(O)(NR¹), S(O)(R¹), or P(O)(R¹); W² is N or C(R²); W³ is N or C(R³); and W⁴ is N or C(R⁴). R⁵, R⁶, R⁷, R⁹, R^{9a}, and R¹⁰ are as described herein.

In an aspect, provided is a compound of Formula (III), or a pharmaceutically acceptable salt or solvate thereof:

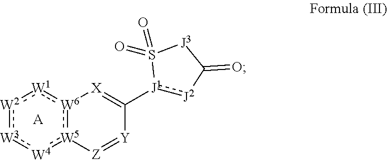

wherein:
W¹ is N, N(R¹), O, C(R), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R);
W³ is N, N(R³), O, C(R³), C(R³)(R^{3a}), C(O), S(O), S(O)₂, S(O)(NR³), S(O)(R³), or P(O)(R);
W⁴ is N, N(R⁴), O, C(R⁴), C(R⁴)(R^{4a}), C(O), S(O), S(O)₂, S(O)(NR⁴), S(O)(R⁴), or P(O)(R);
W⁵ is N or C;
W⁶ is N or C; wherein
   i) at least one of W¹, W², W³, W⁴, W⁵, and W⁶ is N, N(R¹), N(R²), N(R³), N(R⁴), or O; and
   ii) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶
Z is N or C(R⁷)
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NIH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20c}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl;
[symbol] indicates a single or double bond such that all valences are satisfied; and
   provided the compound is not

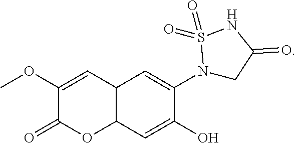 [SMILES: COC1=CC2C=C(N3CC(=O)NS3(=O)=O)C(O)=CC2OC1=O]

In some embodiments, W¹ is N, N(R¹), O, C(R¹), C(R¹)(R^{1a}), C(O), S(O), S(O)₂, S(O)(NR¹), S(O)(R¹), or P(O)(R¹); W² is N, N(R²), O, C(R²), C(R²)(R^{2a}), C(O), S(O), S(O)₂, S(O)(NR²), S(O)(R²), or P(O)(R²); W³ is N, N(R³), O, C(R³), C(R³)(R^{3a}), C(O), S(O), S(O)₂, S(O)(NR³), S(O)(R³), or P(O)(R³); W⁴ is N, N(R⁴), C(R⁴), C(R⁴)(R¹), C(O), S(O), S(O)₂, S(O)(NR⁴), S(O)(R⁴), or P(O)(R⁴). W⁵ is N or C; W⁶ is N or C; wherein i) at least one of W¹, W², W³, W⁴, W⁵, and W⁶ is N, N(R¹), N(R²), N(R³), N(R⁴), or O; and ii) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).

In some embodiments, W¹ is N, N(R¹), O, C(R¹), C(R¹)(R^{1a}), C(O), S(O), S(O)₂, S(O)(NR¹), S(O)(R¹), or P(O)(R¹); W² is N, N(R²), O, C(R²), C(R²)(R^{2a}), C(O), S(O), S(O)₂, S(O)(NR²), S(O)(R²), or P(O)(R²); W³ is N, N(R³), O, C(R³), C(R³)(R^{3a}), S(O), S(O)₂, S(O)(NR³), S(O)(R³), or P(O)(R³); W⁴ is N, N(R⁴), C(R⁴), C(R⁴)(R^{4a}), C(O), S(O), S(O)₂, S(O)(NR⁴), S(O)(R⁴), or P(O)(R⁴); W⁵ is N or C; W⁶ is N or C; wherein i) at least one of W¹, W², W³, W⁴, W⁵, and W⁶ is N, N(R¹), N(R²), N(R³), N(R⁴), or O; and ii) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).

In some embodiments the compound, or a pharmaceutically acceptable salt or solvate thereof, has the structure of Formula (IIIa):

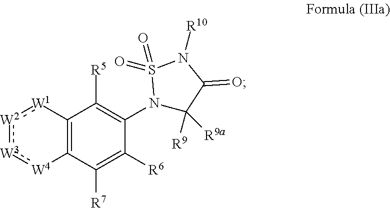

wherein at least one of W¹, W², W³, and W⁴ is N, N(R¹), N(R²), N(R³), N(R⁴), or O and at least one of/W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIIb):

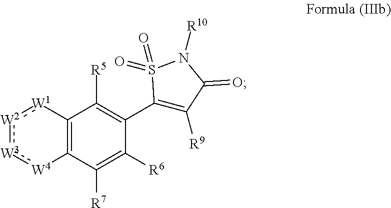

wherein at least one of W¹, W², W³, and W⁴ is N, N(R¹), N(R²), N(R³), N(R⁴), or O and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, and R¹⁰ are as described herein.

In some embodiments the compound, or a pharmaceutically acceptable salt or solvate thereof, has the structure of Formula (IIIc):

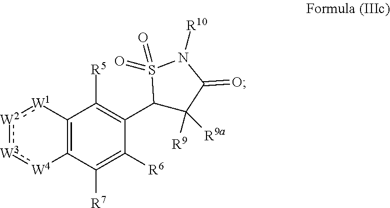

wherein at least one of W¹, W², W³, and W⁴ is N, N(R¹), N(R²), N(R³), N(R⁴), or O and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIId):

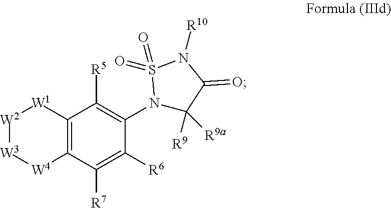

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R²), C(O), S(O), S(O)₂, S(O)(NR²), S(O)(R²), or P(O)(R)
W³ is N(R³), C(R³)(R^{3a}), C(O), S(O), S(O)₂, S(O)(NR³), S(O)(R³), or P(O)(R³)
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N(R¹), N(R²), N(R³), N(R⁴), or O and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
R⁵, R⁶, R⁷, R⁹, R^{9a}, and R¹⁰ are as described herein.

In some embodiments the compound, or a pharmaceutically acceptable salt or solvate thereof, has the structure of Formula (IIIe):

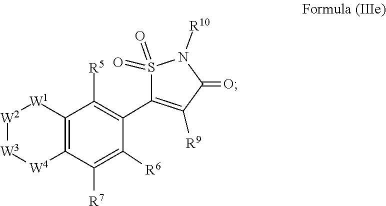

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R^{2a}), C(O), S(O), S(O)₂, S(O)(NR²), S(O)(R²), or P(O)(R²)
W³ is N(R³), C(R³)(R^{3a}), C(O), S(O), S(O)₂, S(O)(NR³), S(O)(R³), or P(O)(R³)
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N(R¹), N(R²), N(R³), N(R⁴), or O and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).

R⁵, R⁶, R⁷, R⁹, and R¹⁰ are as described herein.

In some embodiments the compound, or a pharmaceutically acceptable salt or solvate thereof, has the structure of Formula (IIIf):

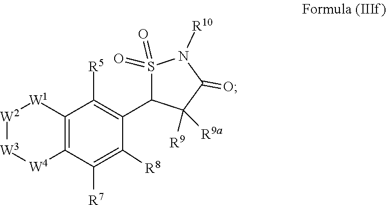

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R^{2a}), C(O), S(O), S(O)₂, S(O)(NR²), S(O)(R²), or P(O)(R²)
W³ is N(R³), C(R³)(R^{3a}), C(O), S(O), S(O)₂, S(O)(NR³), S(O)(R³), or P(O)(R³)
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N(R¹), N(R²), N(R³), N(R⁴), or O and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
R⁵, R⁶, R⁷, R⁹, R^{9a}, and R¹⁰ are as described herein.

In some embodiments the compound, or a pharmaceutically acceptable salt or solvate thereof, has the structure of Formula (IIIg):

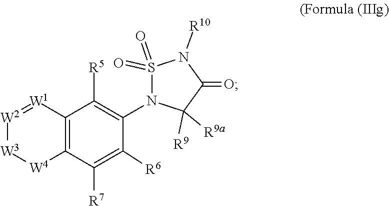

wherein
W¹ is N or C(R¹);
W² is N or C(R²)
W³ is N(R³), C(R³)(R^{3a}), C(O), S(O), S(O)₂, S(O)(NR³), S(O)(R³), or P(O)(R³);
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIIh):

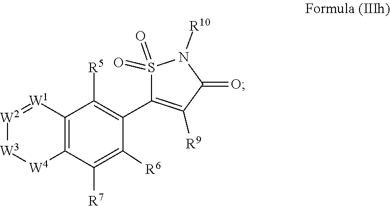

wherein
W¹ is N or C(R¹);
W² is N or C(R²)
W³ is N(R³), C(R³)(R^{3a}), C(O), S(O), S(O)₂, S(O)(NR³), S(O)(R³), or P(O)(R)
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula

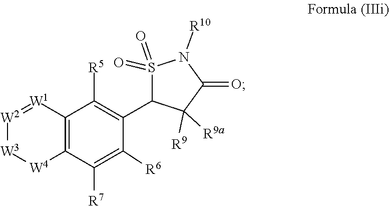

wherein
W¹ is N or C(R¹);
W² is N or C(R²)
W³ is N(R³), C(R³)(R^{3a}), C(O), S(O), S(O)₂, S(O)(NR³), S(O)(R³), or P(O)(R³);
W⁴ is N(R⁴), C(R⁴)(R^{4a}), C(O), S(O), S(O)₂, S(O)(NR⁴), S(O)(R⁴), or P(O)(R⁴); and wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIIj):

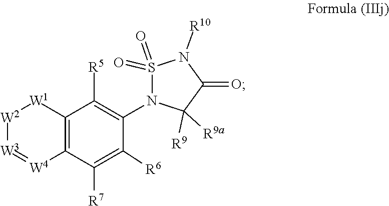

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R²), C(O), S(O), S(O)₂, S(O)(NR²), S(O)(R²), or P(O)(R)
W³ is N or C(R³)
W⁴ is N or C(R⁴); and wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIIk):

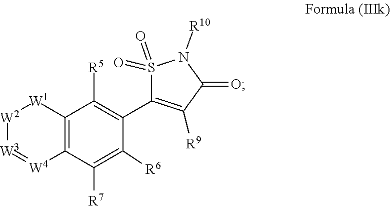

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R^{2a}), C(O), S(O), S(O)₂, S(O)(NR²), S(O)(R²), or P(O)(R²)
W³ is N or C(R³);
W⁴ is N or C(R⁴); and
wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIII):

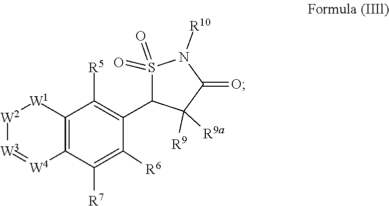

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R²), C(O), S(O), S(O)₂, S(O)(NR²), S(O)(R²), or P(O)(R)
W³ is N or C(R³)
W⁴ is N or C(R⁴); and
wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIIm):

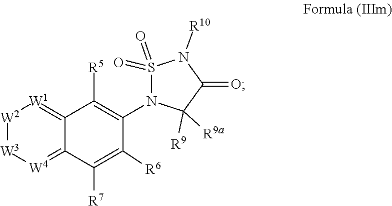

wherein
W¹ is N or C(R¹);
W² is N(R²), C(R²)(R^{2a}), C(O), S(O), S(O)₂, S(O)(NR²), S(O)(R²), or P(O)(R²)
W³ is N(R³), C(R³)(R^{3a}), C(O), S(O), S(O)₂, S(O)(NR³), S(O)(R³), or P(O)(R)
W⁴ is N or C(R⁴); and wherein at least one of W¹, W², W³, and W⁴ is N, N(R¹), N(R²), N(R³), N(R⁴), or O and at least one of W² and W³ is C(O), S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IIIn):

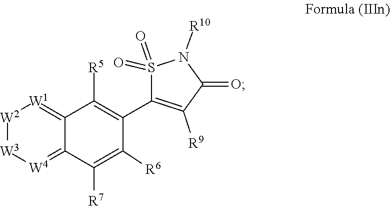

wherein
W¹ is N or C(R¹);
W² is N(R²), C(R²)(R²), C(O), S(O), S(O)₂, S(O)(NR²), S(O)(R²), or P(O)(R)
W³ is N(R³), C(R³)(R^{3a}), C(O), S(O), S(O)₂, S(O)(NR³), S(O)(R³), or P(O)(R)
W⁴ is N or C(R⁴); and
wherein at least one of W¹, W², W³, and W⁴ is N, N(R¹), N(R²), N(R³), N(R⁴), or O and at least one of W² and W³ is C(O), S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IIIo):

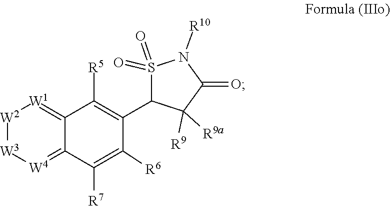

wherein
W¹ is N or C(R¹);
W² is N(R²), C(R²)(R²), C(O), S(O), S(O)₂, S(O)(NR²), S(O)(R²), or P(O)(R)
W³ is N(R³), C(R³)(R^{3a}), C(O), S(O), S(O)₂, S(O)(NR³), S(O)(R³), or P(O)(R)
W⁴ is N or C(R⁴); and
wherein at least one of W¹, W², W³, and W⁴ is N, N(R¹), N(R²), N(R³), N(R⁴), or O and at least one of W² and W³ is C(O), S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IIIp):

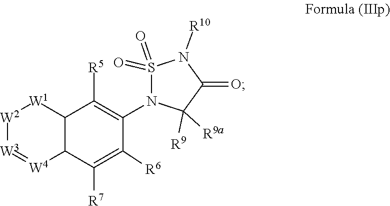

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R^{2a}), C(O), S(O), S(O)₂, S(O)(NR²), S(O)(R²), or P(O)(R)
W³ is N or C(R³)
W⁴ is N or C(R⁴); and
wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIIq):

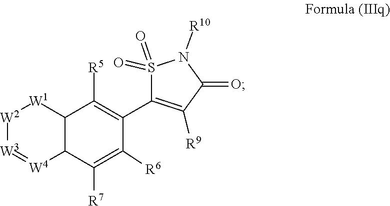

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R²), C(O), S(O), S(O)₂, S(O)(NR²), S(O)(R²), or P(O)(R)
W³ is N or C(R³)
W⁴ is N or C(R⁴); and
wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula

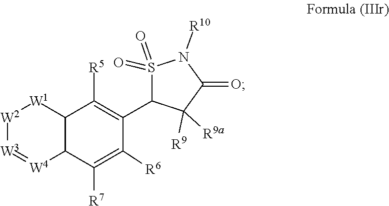

wherein
W¹ is N(R¹), C(R¹)(R^{1a}), C(O), S(O), S(O)₂, S(O)(NR¹), S(O)(R¹), or P(O)(R¹);
W² is N(R²), C(R²)(R^{2a}), C(O), S(O), S(O)₂, S(O)(NR²), S(O)(R²), or P(O)(R²)
W³ is N or C(R³);
W⁴ is N or C(R⁴); and
wherein at least one of W¹, W², W³, and W⁴ is N, N(R¹), N(R²), N(R³), N(R⁴), or O and at least one of W¹ and W² is C(O), S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIIs):

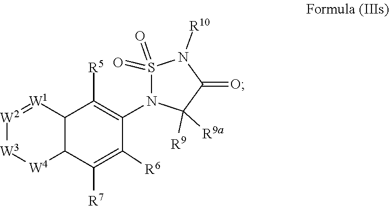

wherein
W¹ is N or C(R¹);
W² is N or C(R²)
W³ is N(R³), C(R³)(R^{3a}), C(O), S(O), S(O)₂, S(O)(NR³), S(O)(R³), or P(O)(R³);
W⁴ is N(R⁴), C(R⁴)(R^{4a}), C(O), S(O), S(O)₂, S(O)(NR⁴), S(O)(R⁴), or P(O)(R⁴); and
wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIIt):

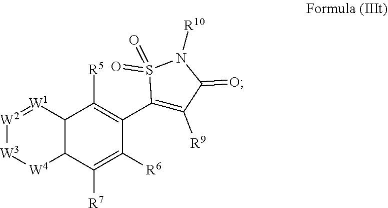

wherein
W¹ is N or C(R¹);
W² is N or C(R²)
W³ is N(R³), C(R³)(R^{3a}), C(O), S(O), S(O)₂, S(O)(NR³), S(O)(R³), or P(O)(R)
W⁴ is N(R⁴), C(R⁴)(R^{4a}), C(O), S(O), S(O)₂, S(O)(NR⁴), S(O)(R⁴), or P(O)(R⁴); and
wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IIIu):

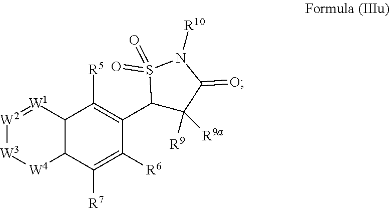

wherein
W¹ is N or C(R¹);
W² is N or CR)
W³ is N(R³), C(R³)(R^{3a}), C(O), S(O), S(O)₂, S(O)(NR³), S(O)(R³), or P(O)(R)
W⁴ is N(R⁴), C(R⁴)(R^{4a}), C(O), S(O), S(O)₂, S(O)(NR⁴), S(O)(R⁴), or P(O)(R⁴); and
wherein at least one of W¹, W², W³, and W⁴ is N, N(R¹), N(R²), N(R³), N(R⁴), or O and at least one of W³, and W⁴ is C(O), S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In an aspect, provided is a compound of Formula (IV), or a pharmaceutically acceptable salt or solvate thereof:

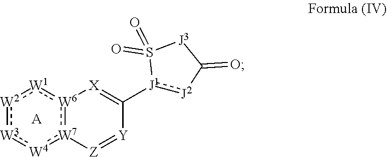

wherein:
W¹ is N, N(R¹), O, C(R¹), C(R¹)(R^{1a}), S(O), S(O)₂, S(O)(NR¹), S(O)(R¹), or P(O)(R¹);
W² is N, N(R²), O, C(R²), C(R²)(R^{2a}), S(O), S(O)₂, S(O)(NR²), S(O)(R²), or P(O)(R²)
W³ is N, N(R³), O, C(R³), C(R³)(R^{3a}), S(O), S(O)₂, S(O)(NR³), S(O)(R³), or P(O)(R³)
W⁴ is N, N(R⁴), O, C(R⁴), C(R⁴)(R^{4a}), S(O), S(O)₂, S(O)(NR⁴), S(O)(R⁴), or P(O)(R⁴).
W⁵ is N or C;
W⁶ is N or C; wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷)
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl; and
[symbol] indicates a single or double bond such that all valences are satisfied.

In some embodiments, W¹ is N, N(R¹), O, C(R¹), C(R¹)(R^{1a}), S(O), S(O)₂, S(O)(NR¹), S(O)(R¹), or P(O)(R¹); W² is N, N(R²), O, C(R²), C(R²)(R^{2a}), S(O), S(O)₂, S(O)(NR²), S(O)(R²), or P(O)(R²); W³ is N, N(R³), O, C(R³), C(R³)(R^{3a}), S(O), S(O)₂, S(O)(NR³), S(O)(R³), or P(O)(R³); W⁴ is N, N(R⁴), C(R⁴), C(R⁴)(R^{4a}), S(O), S(O)₂, S(O)(NR⁴), S(O)(R⁴), or P(O)(R⁴); W⁵ is N or C; W⁶ is N or C; wherein i) at least one of W¹, W², W³, W⁴, W⁵, and W⁶ is N, N(R¹), N(R²), N(R³), N(R⁴), or O; and ii) at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R), or P(O)(R⁴).

In some embodiments the compound, or a pharmaceutically acceptable salt or solvate thereof, has the structure of Formula (IVa):

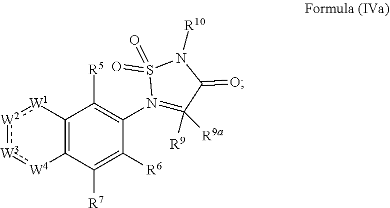

wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVb):

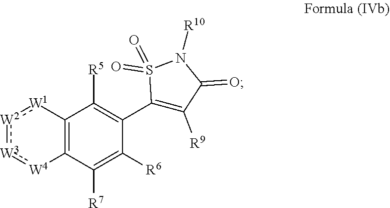

wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, and R¹⁰ are as described herein.

In some embodiments the compound, or a pharmaceutically acceptable salt or solvate thereof, has the structure of Formula (IVc):

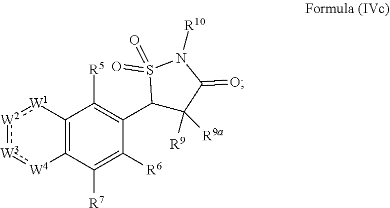

wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVd):

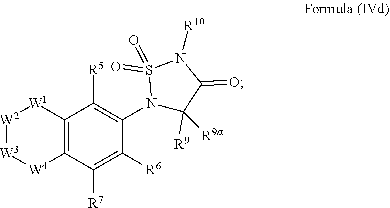

wherein
W¹ is N(R¹), C(R¹)(R^{1a}), S(O), S(O)₂, S(O)(NR¹), S(O)(R¹), or P(O)R¹);
W² is N(R²), C(R²)(R^{2a}), S(O), S(O)₂, S(O)(NR²), S(O)(R²), or PO(R²);
W³ is N(R³), C(R³)(R^{3a}), S(O), S(O)₂, S(O)(NR³), S(O)(R³), or PO(R³);
W⁴ is N(R⁴), C(R⁴)(R^{4a}), S(O), S(O)₂, S(O)(NR⁴), S(O)(R⁴), or P(O)(R⁴); and
wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
R⁵, R⁶, R⁷, R⁹, R^{9a}, and R¹⁰ are as described herein.

In some embodiments the compound, or a pharmaceutically acceptable salt or solvate thereof, has the structure of Formula (IVe):

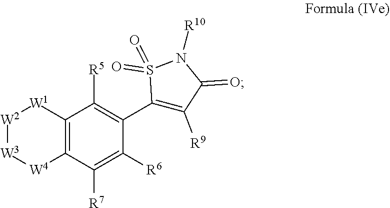

wherein
W¹ is N(R¹), C(R¹)(R^{1a}), S(O), S(O)₂, S(O)(NR¹), S(O)(R¹), or P(O)(R¹);
W² is N(R²), C(R²)(R^{2a}), S(O), S(O)₂, S(O)(NR²), S(O)(R²), or P(O)(R²);
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
   wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
   R⁵, R⁶, R⁷, R⁹, and R¹⁰ are as described herein.

In some embodiments the compound, or a pharmaceutically acceptable salt or solvate thereof, has the structure of Formula (IVf):

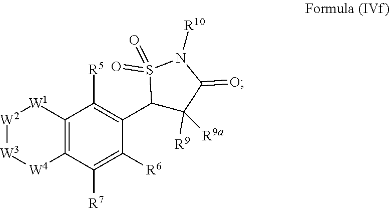

wherein
W¹ is N(R¹), C(R¹)(R^{1a}), S(O), S(O)₂, S(O)(NR¹), S(O)(R¹), or P(O)(R¹);
W² is N(R²), C(R²)(R^{2a}), S(O), S(O)₂, S(O)(NR²), S(O)(R²), or P(O)(R²);
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
   wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
   R⁵, R⁶, R⁷, R⁹, R^{9a}, and R¹⁰ are as described herein.

In some embodiments the compound, or a pharmaceutically acceptable salt or solvate thereof, has the structure of Formula (IVg):

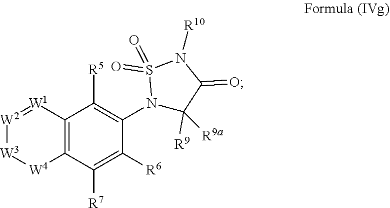

wherein
W¹ is N or C(R¹);
W² is N or C(R²)
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVh):

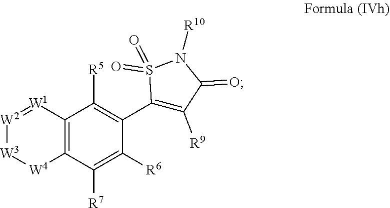

wherein
W¹ is N or C(R¹);
W² is N or C(R²);
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVi):

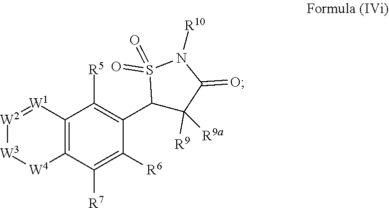

wherein
W¹ is N or C(R¹);
W² is N or C(R²)
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVj):

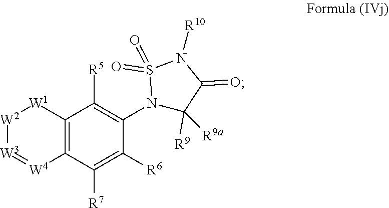

wherein
W¹ is N(R¹), C(R¹)(R^{1a}), S(O), S(O)₂, S(O)(NR¹), S(O)(R¹), or P(O)(R¹);
W² is N(R²), C(R²)(R²), S(O), S(O)₂, S(O)(NR²), S(O)(R²), or P(O)(R²)
W³ is N or C(R³);
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IVk):

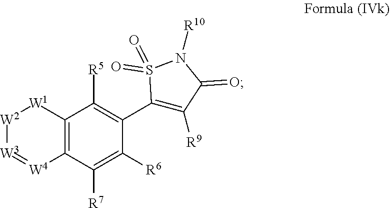

wherein
W¹ is N(R¹), C(R¹)(R^{1a}), S(O), S(O)₂, S(O)(NR¹), S(O)(R¹), or P(O)(R¹);
W² is N(R²), C(R²)(R²), S(O), S(O)₂, S(O)(NR²), S(O)(R²), or P(O)(R)
W³ is N or C(R³)
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IVl):

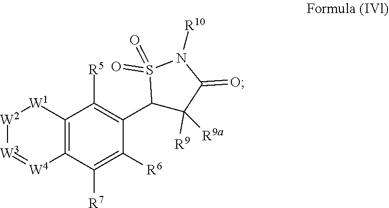

wherein
W¹ is N(R¹), C(R¹)(R^{1a}), S(O), S(O)₂, S(O)(NR¹), S(O)(R¹), or P(O)(R¹);
W² is N(R²), C(R²)(R²), S(O), S(O)₂, S(O)(NR²), S(O)(R²), or P(O)(R)
W³ is N or C(R³)
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IVm):

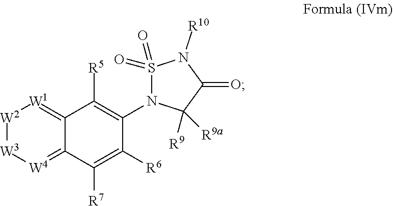

wherein
W¹ is N or C(R¹);
W² is N(R²), C(R²)(R²), S(O), S(O)₂, S(O)(NR²), S(O)(R²), or P(O)(R²);
W³ is N(R³), C(R³)(R^{3a}), S(O), S(O)₂, S(O)(NR³), S(O)(R³), or P(O)(R³);
W⁴ is N or C(R⁴); and
wherein at least one of W² and W³ is S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IVn):

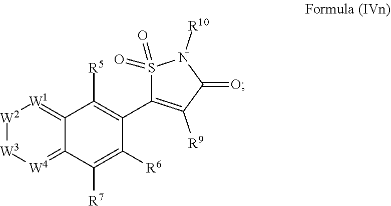

wherein
W¹ is N or C(R¹);
W² is N(R²), C(R²)(R^{2a}), S(O), S(O)₂, S(O)(NR²), S(O)(R²), or P(O)(R²)
W³ is N(R³), C(R³)(R^{3a}), S(O), S(O)₂, S(O)(NR³), S(O)(R³), or P(O)(R³)
W⁴ is N or C(R⁴); and
wherein at least one of W² and W³ is S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IVo):

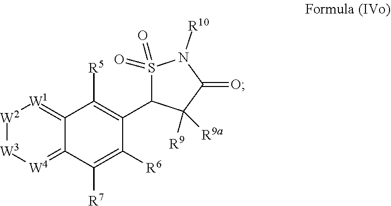

wherein
W¹ is N or C(R¹);
W² is N(R²), C(R²)(R^{2a}), S(O), S(O)₂, S(O)(NR²), S(O)(R²), or P(O)(R²)
W³ is N(R³), C(R³)(R^{3a}), S(O), S(O)₂, S(O)(NR³), S(O)(R³), or P(O)(R³)
W⁴ is N or C(R⁴); and
wherein at least one of W² and W³ is S(O), S(O)₂, S(O)(NR²), S(O)(R²), P(O)(R²), S(O)(NR³), S(O)(R³), or P(O)(R³). R⁵, R⁶, R⁷, R⁹, R^{9a}, and R¹⁰ are as described herein.

In some embodiments the compound, or a pharmaceutically acceptable salt or solvate thereof, has the structure of Formula (IVp):

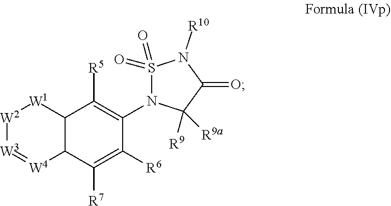

wherein
W¹ is N(R¹), C(R¹)(R^{1a}), S(O), S(O)₂, S(O)(NR¹), S(O)(R¹), or P(O)(R¹);
W² is N(R²), C(R²)(R^{2a}), S(O), S(O)₂, S(O)(NR²), S(O)(R²), or P(O)(R²)
W³ is N or C(R³);
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IVq):

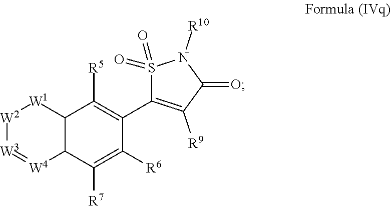

wherein
W¹ is N(R¹), C(R¹)(R^{1a}), S(O), S(O)₂, S(O)(NR¹), S(O)(R¹), or P(O)(R¹);
W² is N(R²), C(R²)(R²), S(O), S(O)₂, S(O)(NR²), S(O)(R²), or P(O)(R)
W³ is N or C(R³)
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IVr):

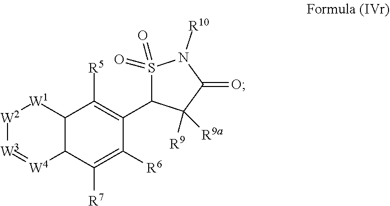

wherein
W¹ is N(R¹), C(R¹)(R^{1a}), S(O), S(O)₂, S(O)(NR¹), S(O)(R¹), or P(O)(R¹);
W² is N(R²), C(R²)(R²), S(O), S(O)₂, S(O)(NR²), S(O)(R²), or P(O)(R²);
W³ is N or C(R³);
W⁴ is N or C(R⁴); and
wherein at least one of W¹ and W² is S(O), S(O)₂, S(O)(NR¹), S(O)(R¹), P(O)(R¹), S(O)(NR²), S(O)(R²), or P(O)(R²). R⁵, R⁶, R⁷, R⁹, R^{9a}, and R¹⁰ are as described herein.

In some embodiments the compound, or a pharmaceutically acceptable salt or solvate thereof, has the structure of Formula (IIIs):

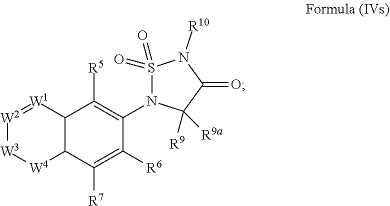

wherein
W¹ is N or C(R¹);
W² is N or CR)
W³ is N(R³), C(R³)(R^{3a}), S(O), S(O)₂, S(O)(NR³), S(O)(R³), or P(O)(R³)
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVt):

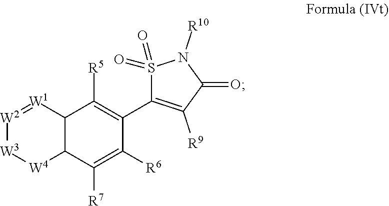

wherein
W¹ is N or C(R¹);
W² is N or C(R²);
W³ is N(R³), C(R³)(R^{3a}), S(O), S(O)₂, S(O)(NR³), S(O)(R³), or P(O)(R³);
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In some embodiments the compound, or a pharmaceutically acceptable salt or solvate thereof, has the structure of Formula (IVu):

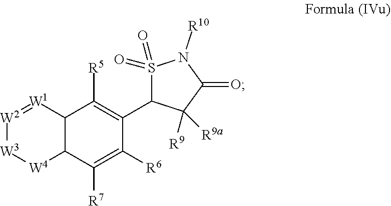

wherein
W¹ is N or C(R¹);
W² is N or CR)
W³ is N(R³), C(R³)(R^{3a}), S(O), S(O)₂, S(O)(NR³), S(O)(R³), or P(O)(R³)
W⁴ is N(R⁴), C(R⁴)(R^{4a}), S(O), S(O)₂, S(O)(NR⁴), S(O)(R⁴), or P(O)(R⁴); and
wherein at least one of W³, and W⁴ is S(O), S(O)₂, S(O)(NR³), S(O)(R³), P(O)(R³), S(O)(NR⁴), S(O)(R⁴), or P(O)(R⁴). R⁵, R⁶, R⁷, R⁹, R^{9a}, and R¹⁰ are as described herein.

In an aspect, provided is a compound of Formula (II), or a pharmaceutically acceptable salt or solvate thereof:

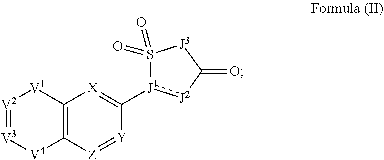

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³)
X is N or C(R⁵);
Y is N or C(R⁶);
Z is N or C(R⁷)
J¹ is N, C, or C(R¹);
J² is N, N(R⁹), C(R⁹), C(R⁹)(R^{9a}), or C(O);
J³ is N(R¹⁰) or C(R¹⁰)(R^{10a});
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl.

In some embodiments the compound, or a pharmaceutically acceptable salt or solvate thereof, has the structure of Formula (IIa):

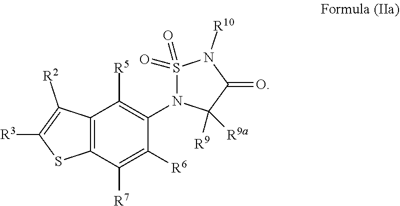

R², R³, R⁵, R⁶, R⁷, R⁹, R^{9a}, and R¹⁰ are as described herein.

In some embodiments the compound, or a pharmaceutically acceptable salt or solvate thereof, has the structure of Formula (IIb):

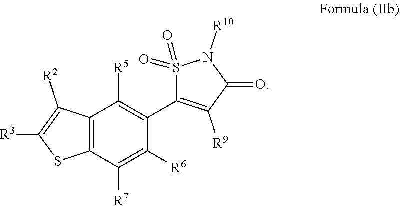

R², R³, R⁵, R¹, R⁷, R⁹, and R¹⁰ are as described herein.

In some embodiments the compound, or a pharmaceutically acceptable salt or solvate thereof, has the structure of Formula (IIc):

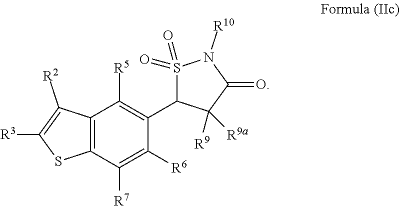

R², R³, R⁵, R⁶, R⁷, R⁹, R^{9a}, and R¹⁰ are as described herein.

In some embodiments the compound, or a pharmaceutically acceptable salt or solvate thereof, has the structure of Formula (IId):

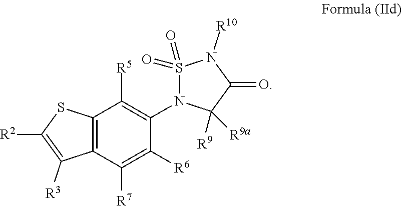

R², R³, R⁵, R⁶, R⁷, R⁹, R^{9a}, and R¹⁰ are as described herein.

In some embodiments the compound, or a pharmaceutically acceptable salt or solvate thereof, has the structure of Formula (IIe):

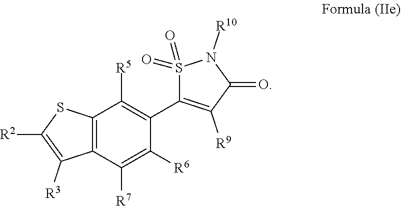

R², R³, R⁵, R⁶, R⁷, R⁹, and R¹⁰ are as described herein.

In some embodiments the compound, or a pharmaceutically acceptable salt or solvate thereof, has the structure of Formula (IIf):

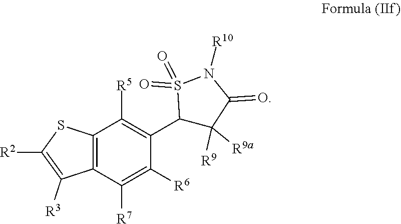

R², R³, R⁵, R⁶, R⁷, R⁹, R^{9a}, and R¹⁰ are as described herein.

In some embodiments the compound, or a pharmaceutically acceptable salt or solvate thereof, has the structure of Formula (IIg):

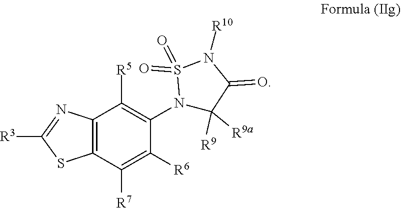

R³, R⁵, R⁶, R⁷, R⁹, R^{9a}, and R¹⁰ are as described herein.

In some embodiments the compound, or a pharmaceutically acceptable salt or solvate thereof, has the structure of Formula (IIh):

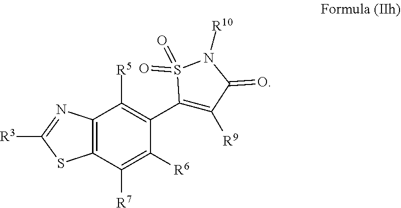

R³, R⁵, R⁶, R⁷, R⁹, and R¹⁰ are as described herein.

In some embodiments the compound, or a pharmaceutically acceptable salt or solvate thereof, has the structure of Formula (IIh):

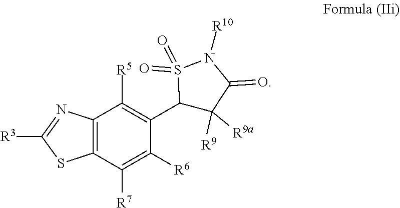

R³, R⁵, R⁶, R⁷, R⁹, R^{9a}, and R¹⁰ are as described herein.

In some embodiments the compound, or a pharmaceutically acceptable salt or solvate thereof, has the structure of Formula (IIj):

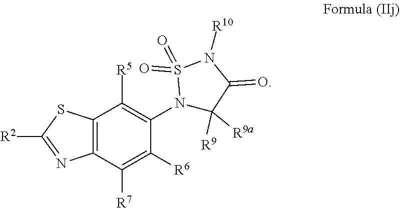

R², R⁵, R⁶, R⁷, R⁹, R^{9a}, and R¹⁰ are as described herein.

In some embodiments the compound, or a pharmaceutically acceptable salt or solvate thereof, has the structure of Formula (IIk):

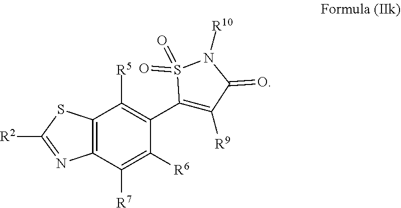

R², R⁵, R⁶, R⁷, R⁹, and R¹⁰ are as described herein.

In some embodiments the compound, or a pharmaceutically acceptable salt or solvate thereof, has the structure of Formula (III):

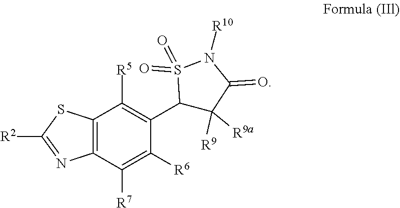

R², R¹, R⁶, R⁷, R⁹, R^{9a}, and R¹⁰ are as described herein.

Sub-formulae of Formula I, II, III, and/or IV are those formula having the same number as the parent formula (e.g., I, II, III, and/or IV) followed by one or more letters. Examples of sub-formulae of Formula I include Ia, Ib, Ic, Id, Ie, If, Ig, Ih, Ii, Ij, Ik, Il, Im, In, Io, Ioa, Iob, Ioc, Ip, Ipa, Ipb, Ipc, Iq, Ir, Is, It, Iu, Iv, Iw, Ix, Iy, Iz, Iaa, Iab, and Iac. Examples of sub-formulae of Formula II include IIa, IIb, IIc, IId, IIe, IIf, IIg, IIh, IIi, IIj, and ILk. Examples of sub-formulae of Formula III Include IIIa, IIIb, IIIc, IIId, bILe, IIIf, IIIg, IIIh, IIIi, IIIj, IIIk, IIII, IIIm, IIIn, IIIo, IIIoa, IIIob, IIIoc, IIIp, IIIpa, IIIpb, IIIpc, IIIq, IIIr, IIIs, IIIt, IIIu, IIIy, IIIw, IIIx, LILy, and IIIz. Examples of sub-formulae of Formula IV Include IVa, IVb, IVc, IVd, IVe, IVf, IVg, IVh, IVi, IVj, IVk, IVI, IVm, IVn, and IVo. Therefore, “Formulae (I), (II), (III), (IV); or sub-formulae thereof” is understood to include a formula selected from Formulae I, Ia, Ib, Ic, Id, Ie, If, Ig, Ih, Ii, Ij, Ik, Il, Im, In, Io, Ioa, Iob, Ioc, Ip, Ipa, Ipb, Ipc, Iq, Ir, Is, It, Iu, Iv, Iw, Ix, Iy, Iz, Iz, Iaa, Iab, Iac, II, IIa, IIb, IIc, IId, IIe, IIf, IIg, IIh, ILi, IIj, IIk, III, IIIa, IIIb, IIIc, IIId, IIIe, IIIf, IIIg, IIIh, IIIi, IIIj, IIIk, IIIl, IIIm, IIIn, IIIo, IIIoa, IIIob, IIIoc, IIIp, IIIpa, IIIpb, IIIpc, IIIq, IIIr, IIIs, IIIt, IIIt, IIIu, IIIv, IIIw, IIIX, LLLy, IIIz, IV, IVa, IVb, IVc, IVd, IVe, IVf, IVg, IVh, IVi, IVj, IVk, IVI, IVm, IVn, IVo, and any other formulae described herein.

In some embodiments of Formula (II), or sub-formulae thereof, V¹ is a bond. In some embodiments of Formula (II), or sub-formulae thereof, V¹ is S. In some embodiments of Formula (II), or sub-formulae thereof, V⁴ is a bond. In some embodiments of Formula (II), or sub-formulae thereof, V⁴ is S.

In some embodiments of Formula (II), or sub-formulae thereof, V¹ is a bond and V⁴ is S. In some embodiments of Formula (II), or sub-formulae thereof, V⁴ is a bond and V¹ is S.

In some embodiments of Formula (II), or sub-formulae thereof, V² is N. In some embodiments of Formula (II), or sub-formulae thereof, V² is C(R²).

In some embodiments of Formula (II), or sub-formulae thereof, V³ is N. In some embodiments of Formula (II), or sub-formulae thereof, V³ is C(R³).

In some embodiments, the compound has Formula:

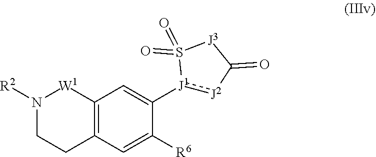

and J¹, J², J³, R², and R⁶ are as described herein. In some embodiments, W¹ is C(O), S(O), S(O)₂, S(O)(NR¹), S(O)(R¹), or P(O)(R¹). In some embodiments, W¹ is C(O). In some embodiments, R⁶ is —OH.

In some embodiments, the compound has Formula

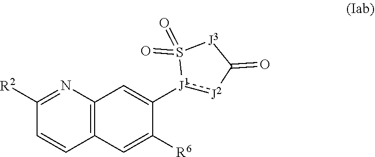

and J¹, J², J³, R², and R⁶ are as described herein. In some embodiments, R⁶ is —OH.

In some embodiments, the compound has Formula:

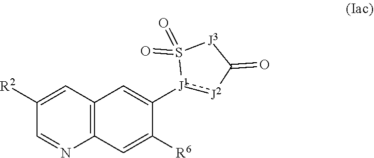

and J¹, J², J³, R², and R⁶ are as described herein. In some embodiments, R⁶ is —OH.

In some embodiments, the compound has Formula:

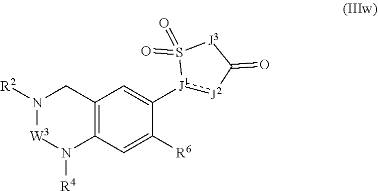

and J¹, J², J³, R², and R⁶ are as described herein. In some embodiments, W³ is C(O), S(O), S(O)₂, S(O)(NR³), S(O)(R³), or P(O)(R³). In some embodiments, W³ is C(O). In some embodiments, R⁶ is —OH. In some embodiments, R⁴ is —CH₃.

In some embodiments, the compound has Formula:

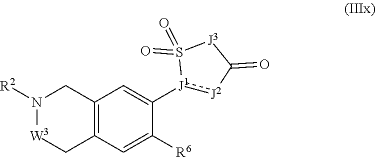

and J¹, J², J³, R², and R⁶ are as described herein. In some embodiments, W³ is C(O), S(O), S(O)₂, S(O)(NR³), S(O)(R³), or P(O)(R³). In some embodiments, W³ is C(O). In some embodiments, R⁶ is —OH.

In some embodiments, the compound has Formula:

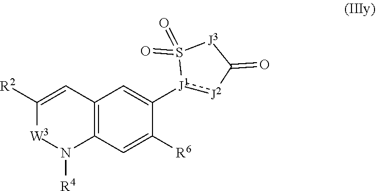

and J¹, J², J³, R², and R⁶ are as described herein. In some embodiments, W³ is C(O), S(O), S(O)₂, S(O)(NR³), S(O)(R³), or P(O)(R³). In some embodiments, W³ is C(O). In some embodiments, R⁶ is —OH. In some embodiments, R⁴ is —CH₃.

In some embodiments, the compound has Formula:

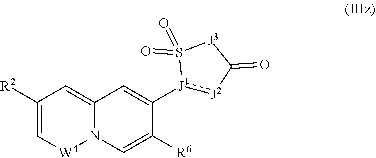

and J¹, J², J³, R², and R⁶ are as described herein. In some embodiments, W⁴ is C(O), S(O), S(O)₂, S(O)(NR⁴), S(O)(R⁴), or P(O)(R⁴). In some embodiments, W⁴ is C(O). In some embodiments, R⁶ is —OH.

In as aspect is provided a compound of Formula (XI), or a pharmaceutically acceptable salt or solvate thereof:

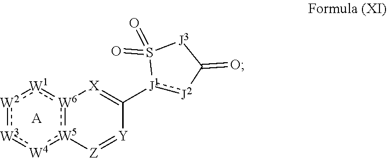

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R);
W⁵ is N, C, or CH;
W⁶ is N, C, or CH;
   wherein at least one of W¹, W², W³, W⁴, W¹, and W⁶ is N, N(R¹), N(R²), N(R³), N(R⁴), or O; and
X is N or C(R⁵);
Y is N or C(R⁶);
Z is N or C(R⁷)
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
R^{12a} is selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², —OC(O)R²⁵, and —P(O)(R²⁵)₂, wherein C₁₋₆alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and provided that:
iv) at least two of Ring A [symbol] are double bonds;
v) when J³ is CH₂; R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f}; and
vi) the compound is not

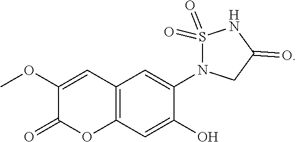 [SMILES: COc1cc2cc(N3CC(=O)NS3(=O)=O)c(O)cc2oc1=O]

In an aspect, provided is a compound of Formula (XIII), or a pharmaceutically acceptable salt or solvate thereof:

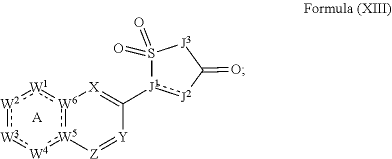

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R^{2a}), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴).
W⁵ is N, C, or CH;
W⁶ is N, C, or CH; wherein
   iii) at least one of W¹, W², W³, W⁴, W⁵, and W⁶ is N, N(R¹), N(R²), N(R³), N(R⁴), or O; and
   iv) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², —OC(O)R²⁵, and —P(O)(R²⁵)₂, wherein C₁₋₆alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉ heteroaryl;
[symbol] indicates a single or double bond such that all valences are satisfied; and
provided the compound is not

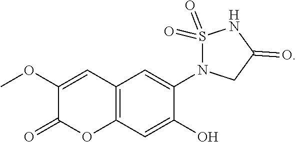 [SMILES: COc1cc2cc(N3CC(=O)NS3(=O)=O)c(O)cc2oc1=O]

In an aspect, provided is a compound of Formula (XII), or a pharmaceutically acceptable salt or solvate thereof:

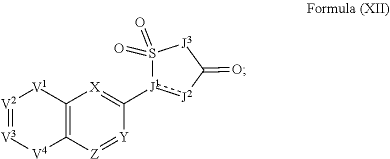

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b};
R³ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20c}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², —OC(O)R²⁵, and —P(O)(R²⁵)₂, wherein C₁₋₆alkyl, C₂₋₆alkenyl, C_{2- 6}alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl; and each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl; and
[symbol] indicates a single or double bond such that all valences are satisfied.

In an aspect, provided is a compound of Formula (XIV), or a pharmaceutically acceptable salt or solvate thereof:

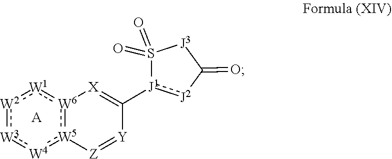

wherein:
W¹ is N, N(R¹), O, C(R¹), C(R¹)(R^{1a}), S(O), S(O)₂, S(O)(NR^{I}), S(O)(R¹), or P(O)(R¹);
W² is N, N(R²), O, C(R²), C(R²)(R^{2a}), S(O), S(O)₂, S(O)(NR²), S(O)(R²), or P(O)(R²);
W³ is N, N(R³), O, C(R³), C(R³)(R^{3a}), S(O), S(O)₂, S(O)(NR³), S(O)(R³), or P(O)(R³);
W⁴ is N, N(R⁴), O, C(R⁴), C(R⁴)(R^{4a}), S(O), S(O)₂, S(O)(NR⁴), S(O)(R⁴), or P(O)(R⁴);
W⁵ is N, C, or CH;
W⁶ is N, C, or CH; wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆ alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², —OC(O)R²⁵, and —P(O)(R²⁵)₂, wherein C₁₋₆alkyl, C₂₋₆alkenyl, C₂₋₆alkynyl, C₃₋₁₀cycloalkyl, —CH₂—C₃₋₁₀cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In some embodiments of a compound of Formula (I) or (XI), the compound is a compound of Formula (I′), or a pharmaceutically acceptable salt or solvate thereof:

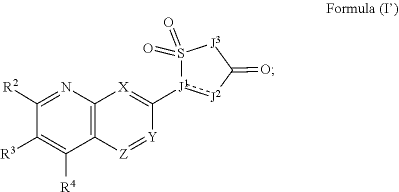

wherein:
X is N or C(R⁵);
Y is N or C(R⁶);
Z is N or C(R⁷)
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁴ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
R^{12a} is selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{61b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and
provided that when J³ is CH₂; R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}.

In embodiments of Formula I′, R⁵ is —F and R⁶ is —OH.

In some embodiments, the compound is a compound of Formula (Ia′):

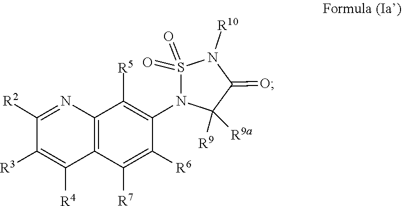

R², R³, R⁴, R⁵, R⁶, R⁷, R⁹, R^{9a}, and R¹⁰ are as described herein. In embodiments of Formula Ia′, R⁵ is —F and R⁶ is —OH.

In some embodiments, the compound is a compound of Formula (Ib′):

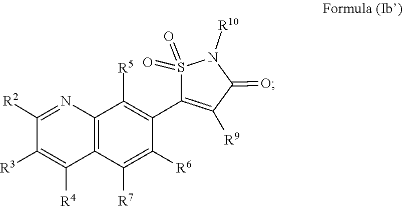

R², R³, R⁴, R⁵, R⁶, R⁷, R⁹, R^{9a}, and R¹⁰ are as described herein. In embodiments of Formula Ib′, R⁵ is —F and R⁶ is —OH.

In some embodiments, the compound is a compound of Formula (Ic′):

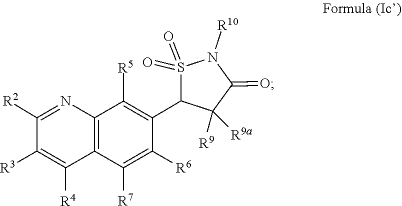

R², R³, R⁴, R⁵, R⁶, R⁷, R⁹, R^{9a}, and R¹⁰ are as described herein. In embodiments of Formula Ic′, R⁵ is —F and R⁶ is —OH.

In some embodiments, the compound is a compound of Formula (Ia′-1):

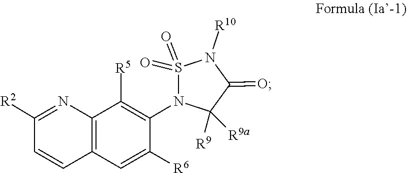

R², R³, R⁴, R⁵, R⁶, R⁷, R⁹, R^{9a}, and R¹⁰ are as described herein. In embodiments of Formula Ia′-1, R⁵ is —F and R⁶ is —OH.

In some embodiments, the compound is a compound of Formula (Ib′-1):

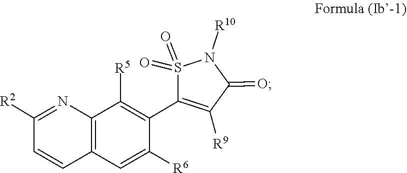

R², R⁵, R⁶, R⁹, R^{9a}, and R¹⁰ are as described herein. In embodiments of Formula Ib′-1, R⁵ is —F and R⁶ is —OH.

In some embodiments, the compound is a compound of Formula (Ic′-1):

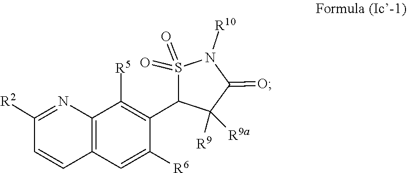

R², R⁵, R⁶, R⁹, R^{9a}, and R¹⁰ are as described herein. In embodiments of Formula Ic′-1, R⁵ is —F and R⁶ is —OH.

In some embodiments, the compound is a compound of Formula (Ia′-2):

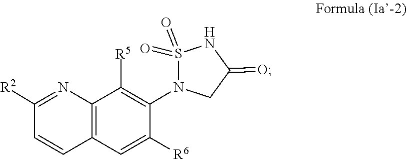

R², R⁵, and R⁶ are as described herein. In embodiments of Formula Ia′-2, R⁵ is —F and R⁶ is —OH.

In some embodiments, the compound is a compound of Formula (Ib′-2):

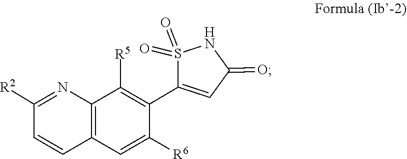

R², R⁵, and R⁶ are as described herein. In embodiments of Formula Ib′-2, R⁵ is —F and R⁶ is —OH.

In some embodiments, the compound is a compound of Formula (Ic′-2):

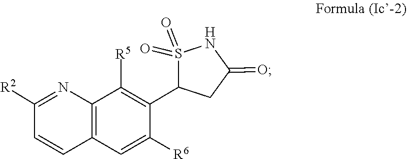

R², R⁵, and R⁶ are as described herein. In embodiments of Formula Ic′-2, R⁵ is —F and R⁶ is —OH.

In some embodiments of a compound of Formula (I) or (XI), the compound is a compound of Formula (I″), or a pharmaceutically acceptable salt or solvate thereof:

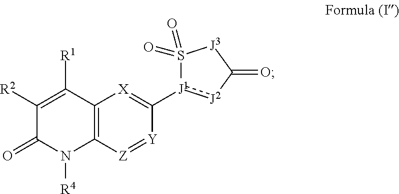

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R⁴ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{61b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C_{1- 6}alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiment of Formula I″, R⁵ is —F and R⁶ is —OH.

In some embodiments, the compound is a compound of Formula (Ia″):

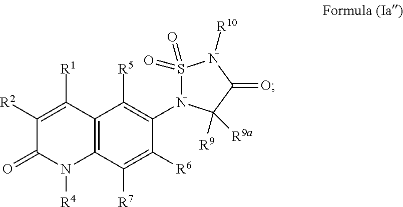

R¹, R², R⁴, R⁵, R⁶, R⁷, R⁹, R^{9a}, and R¹⁰ are as described herein. In embodiments of Formula Ia″, R⁵ is —F and R⁶ is —OH.

In some embodiments, the compound is a compound of Formula (Ib″):

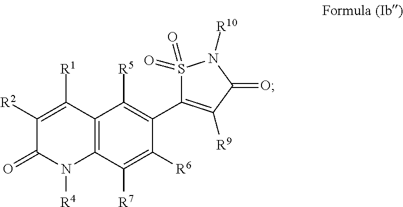

R¹, R², R⁴, R⁵, R⁶, R⁷, R⁹, R^{9a}, and R¹⁰ are as described herein. In embodiments of Formula Ib″, R⁵ is —F and R⁶ is —OH.

In some embodiments, the compound is a compound of Formula (Ic″):

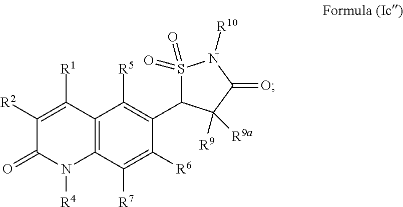

R¹, R², R⁴, R⁵, R⁶, R⁷, R⁹, R^{9a}, and R¹⁰ are as described herein. In embodiments of Formula Ic″, R⁵ is —F and R⁶ is —OH.

In some embodiments, the compound is a compound of Formula (Ia″-1):

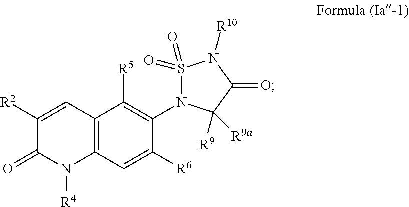

R², R⁴, R⁵, R⁶, R⁹, R^{9a}, and R¹⁰ are as described herein. In embodiments of Formula Ia″-1, R⁵ is —F and R⁶ is —OH.

In some embodiments, the compound is a compound of Formula (Ib″-1):

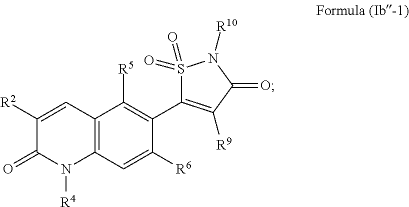

R², R⁴, R⁵, R⁶, R⁹, R^{9a}, and R¹⁰ are as described herein. In embodiments of Formula Ib″-1, R⁵ is —F and R⁶ is —OH.

In some embodiments, the compound is a compound of Formula (Ic″-1):

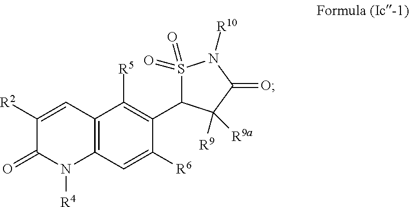

R², R⁴, R⁵, R⁶, R⁹, R^{9a}, and R¹⁰ are as described herein. In embodiments of Formula Ic″-1, R⁵ is —F and R⁶ is —OH.

In some embodiments, the compound is a compound of Formula (Ia″-2):

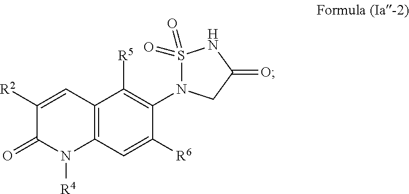

R², R⁴, R⁵, and R⁶ are as described herein. In embodiments of Formula Ia″-2, R⁵ is —F and R⁶ is —OH.

In some embodiments, the compound is a compound of Formula (Ib″-2):

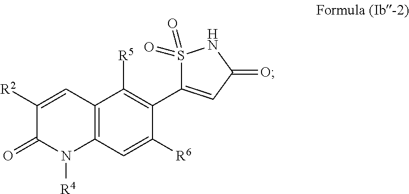

R², R⁴, R⁵, and R⁶ are as described herein. In embodiments of Formula Ib″-2, R⁵ is —F and R⁶ is —OH.

In some embodiments, the compound is a compound of Formula (Ic″-2):

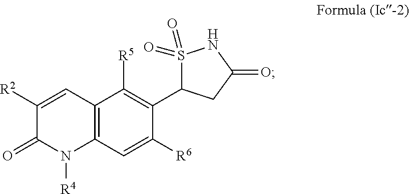

R², R⁴, R⁵, and R⁶ are as described herein. In embodiments of Formula Ic″-2, R⁵ is —F and R⁶ is —OH.

In some embodiments of a compound of Formula (I) or (XI), the compound is a compound of Formula (I′″), or a pharmaceutically acceptable salt or solvate thereof:

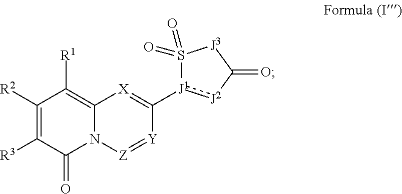

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three
R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
R^{12a} is selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{61b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and
provided that when J³ is CH₂; R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}.

In embodiments of Formula I′″, R⁵ is —F and R⁶ is —OH.

In some embodiments, the compound is a compound of Formula (Ia′″):

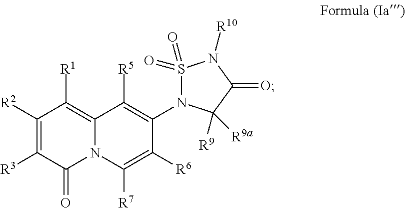

R¹, R², R³, R⁵, R⁶, R⁷, R⁹, R^{9a}, and R¹⁰ are as described herein. In embodiments of Formula Ia′″, R⁵ is —F and R⁶ is —OH.

In some embodiments, the compound is a compound of Formula (Ib′″):

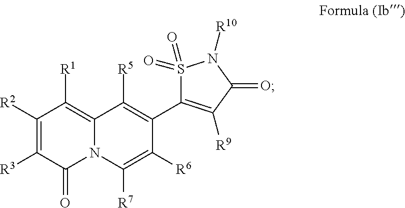

R¹, R², R³, R⁵, R⁶, R⁷, R⁹, R^{9a}, and R¹⁰ are as described herein. In embodiments of Formula Ib′″, R⁵ is —F and R⁶ is —OH.

In some embodiments, the compound is a compound of Formula (Ic′″):

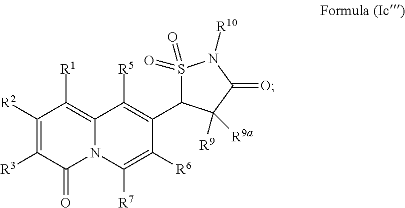

R¹, R², R³, R⁵, R⁶, R⁷, R⁹, R^{9a}, and R¹⁰ are as described herein. In embodiments of Formula Ic′″, R⁵ is —F and R⁶ is —OH.

In some embodiments, the compound is a compound of Formula (Ia′″-1):

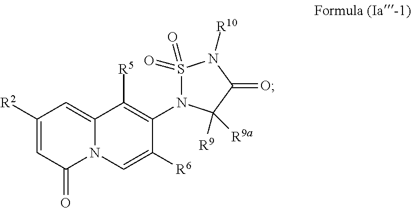

R², R³, R⁵, R⁶, R⁷, R⁹, R^{9a}, and R¹⁰ are as described herein. In embodiments of Formula Ia′″-1, R⁵ is —F and R⁶ is —OH.

In some embodiments, the compound is a compound of Formula (Ib′″-1):

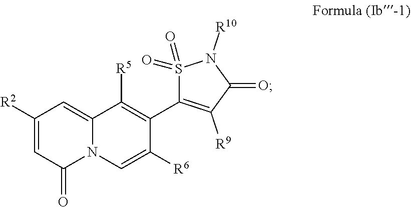

R², R⁵, R⁶, R⁹, R^{9a}, and R¹⁰ are as described herein. In embodiments of Formula Ib′″-1, R⁵ is —F and R⁶ is —OH.

In some embodiments, the compound is a compound of Formula (Ic′″-1):

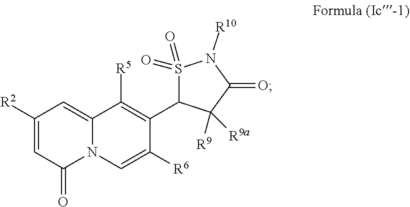

R², R⁵, R⁶, R⁹, R^{9a}, and R¹⁰ are as described herein. In embodiments of Formula Ic′″-1, R⁵ is —F and R⁶ is —OH.

In some embodiments, the compound is a compound of Formula (Ia′″-2):

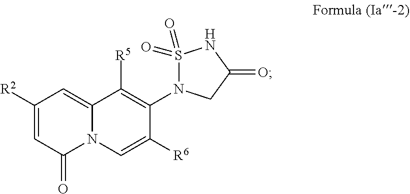

R², R⁵, and R⁶ are as described herein. In embodiments of Formula Ib′″-2, R⁵ is —F and R⁶ is —OH.

In some embodiments, the compound is a compound of Formula (Ic′″-2):

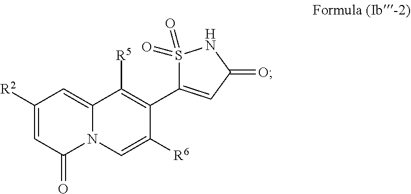

R², R⁵, and R⁶ are as described herein. In embodiments of Formula Ib′″-2, R⁵ is —F and R⁶ is —OH.

In some embodiments, the compound is a compound of Formula (Ic′″-2):

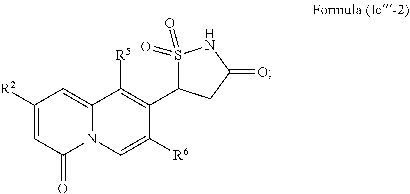

R², R⁵, and R⁶ are as described herein. In embodiments of Formula Ic′″-2, R⁵ is —F and R⁶ is —OH.

In some embodiments of a compound of Formula (III) or (XIII), the compound is a compound of Formula (III′), or a pharmaceutically acceptable salt or solvate thereof:

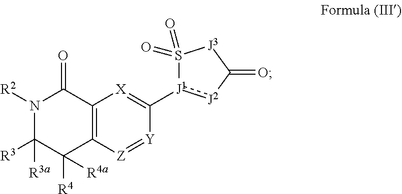

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR¹⁶ⁱ*) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C_{1- 6}alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiments of Formula III′, R⁵ is —F and R⁶ is —OH.

In some embodiments, the compound is a compound of Formula (IIIa′):

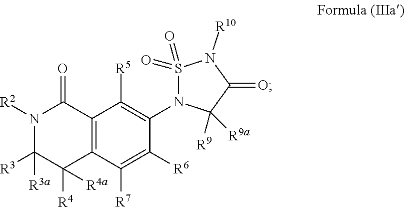

R², R³, R^{3a}, R⁴, R^{4a}, R⁵, R⁶, R⁷, R⁹, R^{9a}, and R¹⁰ are as described herein. In embodiments of Formula IIIa′, R⁵ is —F and R⁶ is —OH.

In some embodiments, the compound is a compound of Formula (IIIb′):

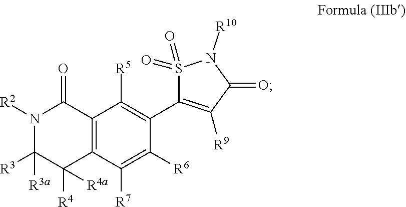

R², R³, R^{3a}, R⁴, R^{4a}, R⁵, R⁶, R⁷, R⁹, R^{9a}, and R¹⁰ are as described herein. In embodiments of Formula IIIb′, R⁵ is —F and R⁶ is —OH.

In some embodiments, the compound is a compound of Formula (IIIc′):

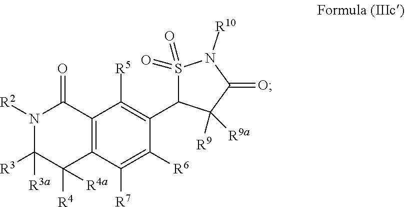

R², R³, R^{3a}, R⁴, R^{4a}, R⁵, R⁶, R⁷, R⁹, R^{9a}, and R¹⁰ are as described herein. In embodiments of Formula IIIc′, R⁵ is —F and R⁶ is —OH.

In some embodiments, the compound is a compound of Formula (IIIa′-1):

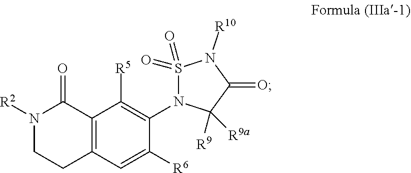

R², R⁵, R⁶, R⁹, R^{9a}, and R¹⁰ are as described herein. In embodiments of Formula IIIa′-1, R⁵ is —F and R⁶ is —OH.

In some embodiments, the compound is a compound of Formula (IIIb′-1):

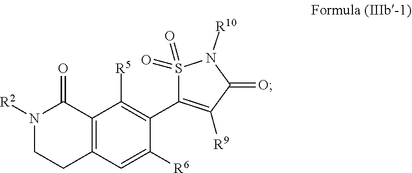

R², R⁵, R⁶, R⁹, R^{9a}, and R¹⁰ are as described herein. In embodiments of Formula IIIb′-1, R⁵ is —F and R⁶ is —OH.

In some embodiments, the compound is a compound of Formula (IIIc′-1):

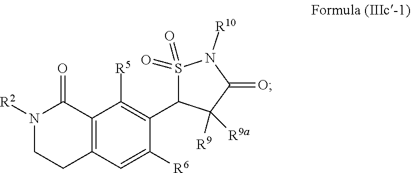

R², R⁵, R⁶, R⁹, R^{9a}, and R¹⁰ are as described herein. In embodiments of Formula IIIc′-1, R⁵ is —F and R⁶ is —OH.

In some embodiments, the compound is a compound of Formula (IIIa′-2):

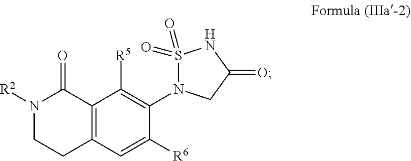

R², R⁵, and R⁶ are as described herein. In embodiments of Formula IIIa′-2, R⁵ is —F and R⁶ is —OH.

In some embodiments, the compound is a compound of Formula (IIIb′-2):

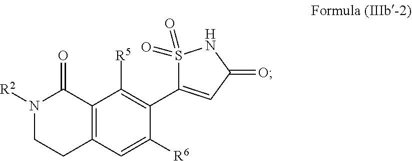

R², R⁵, and R⁶ are as described herein. In embodiments of Formula IIIb′-2, R⁵ is —F and R⁶ is —OH.

In some embodiments, the compound is a compound of Formula (IIIc′-2):

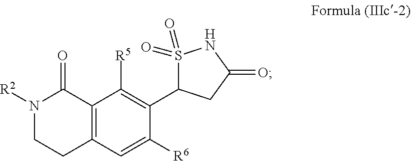

R², R⁵, and R⁶ are as described herein. In embodiments of Formula IIIc′-2, R⁵ is —F and R⁶ is —OH.

In some embodiments of a compound of Formula (III) or (XIII), the compound is a compound of Formula (III″), or a pharmaceutically acceptable salt or solvate thereof:

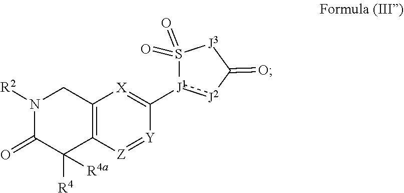

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁_.heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiments of Formula III″, R⁵ is —F and R⁶ is —OH.

In some embodiments, the compound is a compound of Formula (IIIa″):

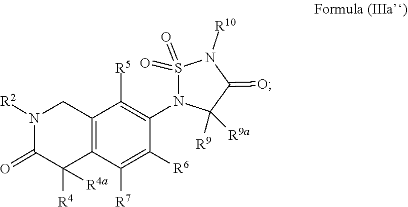

R², R⁴, R^{4a}, R⁵, R⁶, R⁷, R⁹, R^{9a}, and R¹⁰ are as described herein. In embodiments of Formula IIIa″, R⁵ is —F and R⁶ is —OH.

In some embodiments, the compound is a compound of Formula (IIIb″):

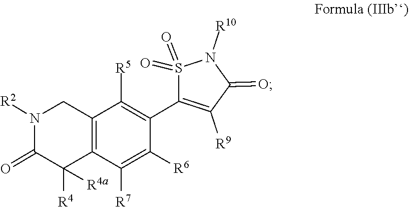

R², R⁴, R^{4a}, R⁵, R⁶, R⁷, R⁹, R^{9a}, and R¹⁰ are as described herein. In embodiments of Formula IIIb″, R⁵ is —F and R⁶ is —OH.

In some embodiments, the compound is a compound of Formula (IIIc″):

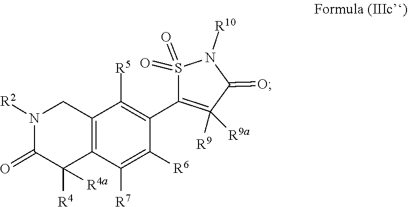

R², R⁴, R^{4a}, R⁵, R⁶, R⁷, R⁹, R^{9a}, and R¹⁰ are as described herein. In embodiments of Formula IIIc″, R⁵ is —F and R⁶ is —OH.

In some embodiments, the compound is a compound of Formula (IIIa″-1):

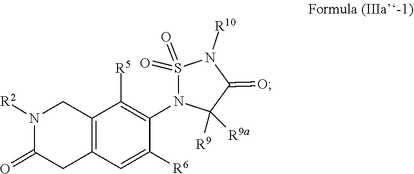

R², R⁵, R⁶, R⁹, R^{9a}, and R¹⁰ are as described herein. In embodiments of Formula IIIa″-1, R⁵ is —F and R⁶ is —OH.

In some embodiments, the compound is a compound of Formula (IIIb″-1):

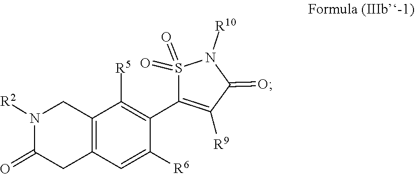

R², R⁵, R⁶, R⁹, R^{9a}, and R¹⁰ are as described herein. In embodiments of Formula IIIb″-1, R⁵ is —F and R⁶ is —OH.

In some embodiments, the compound is a compound of Formula (IIIc″-1):

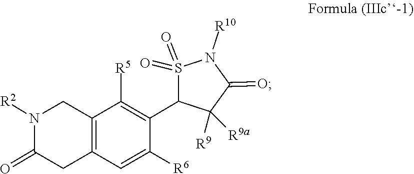

R², R⁵, R⁶, R⁹, R^{9a}, and R¹⁰ are as described herein. In embodiments of Formula IIIc″-1, R⁵ is —F and R⁶ is —OH.

In some embodiments, the compound is a compound of Formula (IIIa″-2):

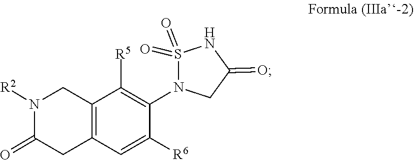

R², R⁵, and R⁶ are as described herein. In embodiments of Formula IIIa″-2, R⁵ is —F and R⁶ is —OH.

In some embodiments, the compound is a compound of Formula (IIIb″-2):

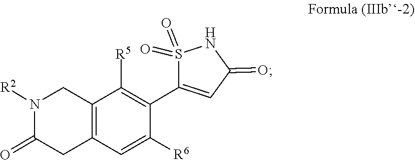

R², R⁵, and R⁶ are as described herein. In embodiments of Formula IIIb″-2, R⁵ is —F and R⁶ is —OH.

In some embodiments, the compound is a compound of Formula (IIIc″-2):

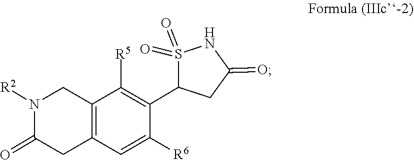

R², R⁵, and R⁶ are as described herein. In embodiments of Formula IIIc″-2, R⁵ is —F and R⁶ is —OH.

In some embodiments of a compound of Formula (III) or (XIII), the compound is a compound of Formula (III′″), or a pharmaceutically acceptable salt or solvate thereof:

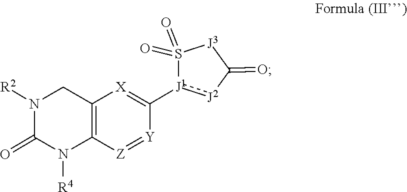

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R⁴ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{61b}) and —P(O)(OR^{16a})(OR^{61b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiments of Formula III′″, R⁵ is —F and R⁶ is —OH.

In some embodiments, the compound is a compound of Formula (IIIa′″):

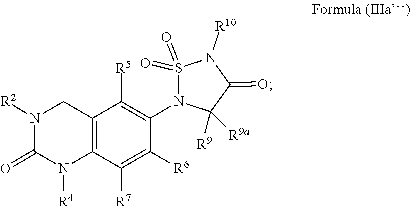

R², R⁴, R⁵, R⁶, R⁷, R⁹, R^{9a}, and R¹⁰ are as described herein. In embodiments of Formula IIIa′″, R⁵ is —F and R⁶ is —OH.

In some embodiments, the compound is a compound of Formula (IIIb′″):

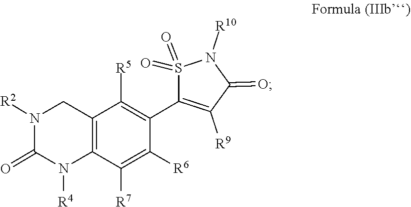

R², R⁴, R⁵, R⁶, R⁷, R⁹, R^{9a}, and R¹⁰ are as described herein. In embodiments of Formula IIIb′″, R⁵ is —F and R⁶ is —OH.

In some embodiments, the compound is a compound of Formula (IIIc′″):

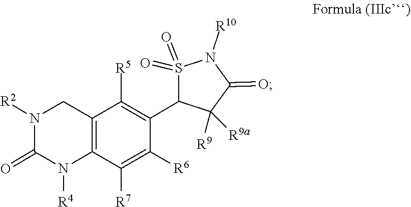

R², R⁴, R⁵, R⁶, R⁷, R⁹, R^{9a}, and R¹⁰ are as described herein. In embodiments of Formula IIIc′″, R⁵ is —F and R⁶ is —OH.

In some embodiments, the compound is a compound of Formula (IIIa′″-2):

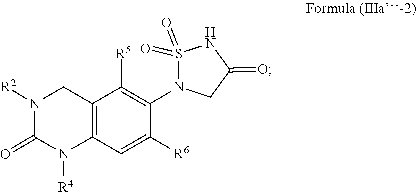

R², R⁴, R⁵, and R⁶ are as described herein. In embodiments of Formula IIIa′″-2, R⁵ is —F and R⁶ is —OH.

In some embodiments, the compound is a compound of Formula (IIIb′″-2):

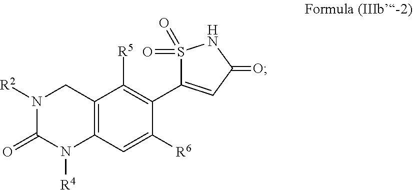

R², R⁴, R⁵, and R⁶ are as described herein. In embodiments of Formula IIIb′″-2, R⁵ is —F and R⁶ is —OH.

In some embodiments, the compound is a compound of Formula (IIIc′″-2):

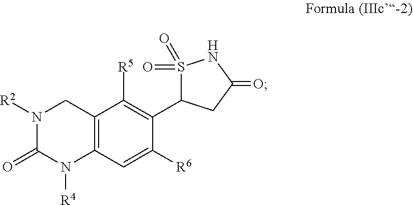

R², R⁴, R⁵, and R⁶ are as described herein. In embodiments of Formula IIIc′″-2, R⁵ is —F and R⁶ is —OH.

In some embodiments of a compound of Formula (III) or (XIII), the compound is a compound of Formula (III) or a pharmaceutically acceptable salt or solvate thereof:

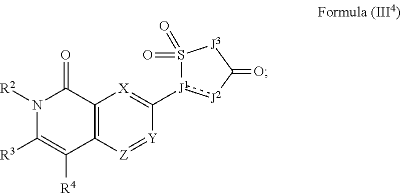

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁴ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C_{1- 6}alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiments of Formula III⁴, R⁵ is —F and R⁶ is —OH.

In some embodiments, the compound is a compound of Formula (IIIa⁴):

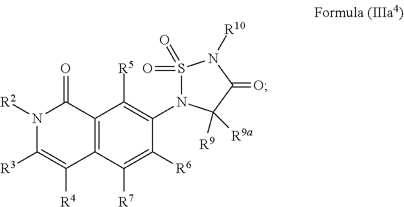

R², R³, R⁴, R⁵, R⁶, R⁷, R⁹, R^{9a}, and R¹⁰ are as described herein. In embodiments of Formula IIIa⁴, R⁵ is —F and R⁶ is —OH.

In some embodiments, the compound is a compound of Formula (IIIb⁴):

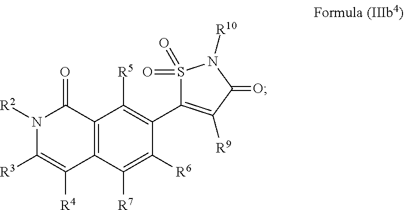

R², R³, R⁴, R⁵, R⁶, R⁷, R⁹, R^{9a}, and R¹⁰ are as described herein. In embodiments of Formula IIIb⁴, R⁵ is —F and R⁶ is —OH.

In some embodiments, the compound is a compound of Formula (IIIc⁴):

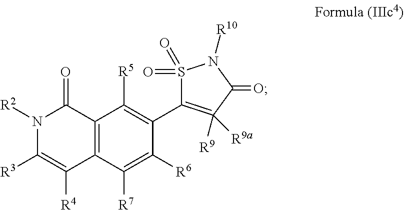

R², R³, R⁴, R⁵, R⁶, R⁷, R⁹, R^{9a}, and R¹⁰ are as described herein. In embodiments of Formula IIIc⁴, R⁵ is —F and R⁶ is —OH.

In some embodiments, the compound is a compound of Formula (IIIa⁴-1):

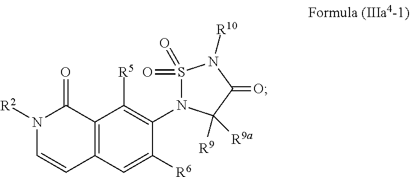

R², R⁵, R⁶, R⁹, R^{9a}, and R¹⁰ are as described herein. In embodiments of Formula IIIa⁴-1, R⁵ is —F and R⁶ is —OH.

In some embodiments, the compound is a compound of Formula (IIIb⁴-1):

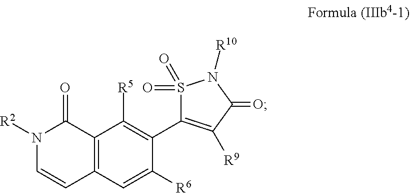

R², R⁵, R⁶, R⁹, R^{9a}, and R¹⁰ are as described herein. In embodiments of Formula IIIb⁴-1, R⁵ is —F and R⁶ is —OH.

In some embodiments, the compound is a compound of Formula (IIIc⁴-1):

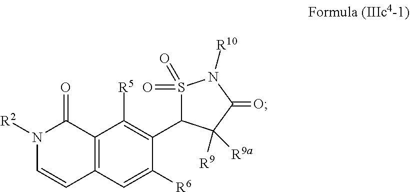

R², R⁵, R⁶, R⁹, R^{9a}, and R¹⁰ are as described herein. In embodiments of Formula IIIc⁴-1, R⁵ is —F and R⁶ is —OH.

In some embodiments, the compound is a compound of Formula (IIIa⁴-2):

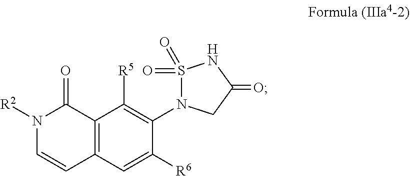

R², R⁵, and R⁶ are as described herein. In embodiments of Formula IIIa⁴-2, R⁵ is —F and R⁶ is —OH.

In some embodiments, the compound is a compound of Formula (IIIb⁴-2):

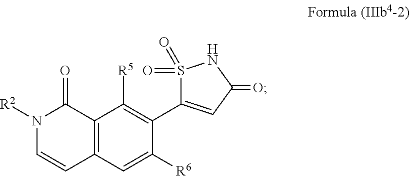

R², R⁵, and R⁶ are as described herein. In embodiments of Formula IIIb⁴-2, R⁵ is —F and R⁶ is —OH.

In some embodiments, the compound is a compound of Formula (IIIc⁴-2):

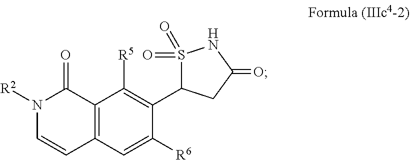

R², R⁵, and R⁶ are as described herein. In embodiments of Formula IIIc⁴-2, R⁵ is —F and R⁶ is —OH.

In some embodiments of a compound of Formula (III) or (XIII), the compound is a compound of Formula (III⁵), or a pharmaceutically acceptable salt or solvate thereof:

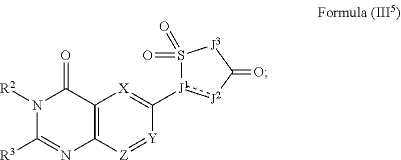

wherein:
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20b}, R^{20c}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl; and
[symbol] indicates a single or double bond such that all valences are satisfied.

In embodiments of Formula III⁵, R⁵ is —F and R⁶ is —OH.

In some embodiments, the compound is a compound of Formula (IIIa⁵):

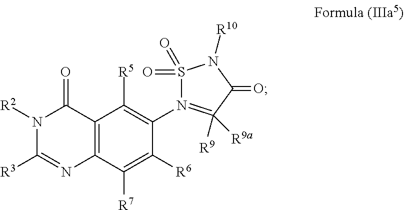

R², R³, R⁵, R⁶, R⁷, R⁹, R^{9a}, and R¹⁰ are as described herein. In embodiments of Formula IIIa⁵, R⁵ is —F and R⁶ is —OH.

In some embodiments, the compound is a compound of Formula (IIIb⁵):

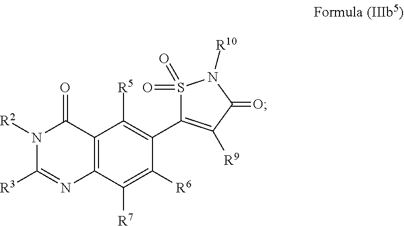

R², R³, R⁵, R⁶, R⁷, R⁹, R^{9a}, and R¹⁰ are as described herein. In embodiments of Formula IIIb⁵, R⁵ is —F and R⁶ is —OH.

In some embodiments, the compound is a compound of Formula (IIIc⁵):

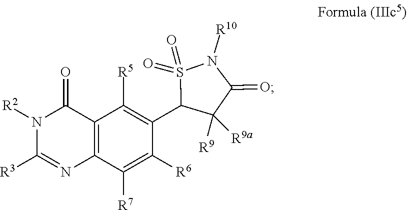

R², R³, R⁵, R⁶, R⁷, R⁹, R^{9a}, and R¹⁰ are as described herein. In embodiments of Formula IIIc⁵, R⁵ is —F and R⁶ is —OH.

In some embodiments, the compound is a compound of Formula (IIIa⁵-1):

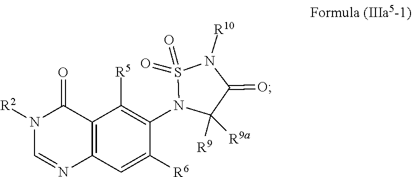

R², R⁵, R⁶, R⁹, R^{9a}, and R¹⁰ are as described herein. In embodiments of Formula IIIa⁵-1, R⁵ is —F and R⁶ is —OH.

In some embodiments, the compound is a compound of Formula (IIIb⁵-1):

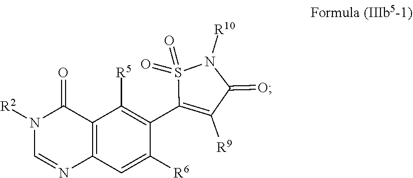

R², R⁵, R⁶, R⁹, R^{9a}, and R¹⁰ are as described herein. In embodiments of Formula IIIb⁵-1, R⁵ is —F and R⁶ is —OH.

In some embodiments, the compound is a compound of Formula (IIIc⁵-1):

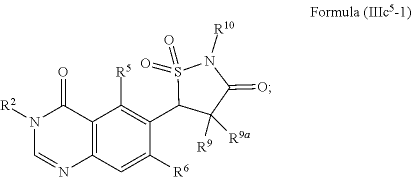

R², R⁵, R⁶, R⁹, R^{9a}, and R¹⁰ are as described herein. In embodiments of Formula IIIc⁵-1, R⁵ is —F and R⁶ is —OH.

In some embodiments, the compound is a compound of Formula (IIIa⁵-2):

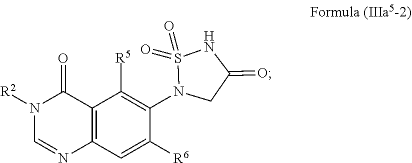

R², R⁵, and R⁶ are as described herein. In embodiments of Formula IIIa⁵-2, R⁵ is —F and R⁶ is —OH.

In some embodiments, the compound is a compound of Formula (IIIb⁵-2):

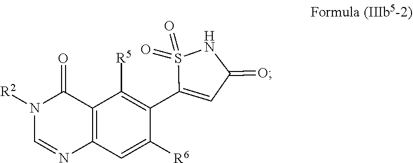

R², R⁵, and R⁶ are as described herein. In embodiments of Formula IIIb⁵-2, R⁵ is —F and R⁶ is —OH.

In some embodiments, the compound is a compound of Formula (IIIc⁵-2):

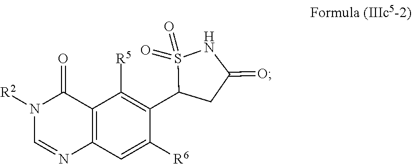

R², R⁵, and R⁶ are as described herein. In embodiments of Formula IIIc⁵-2, R⁵ is —F and R⁶ is —OH.

In some embodiments, W¹ is N, N(R¹), C(R¹), C(R¹)(R^{1a}), C(O), S(O), S(O)₂, S(O)(NR¹), S(O)(R¹), or P(O)(R¹). In some embodiments, W² is N, N(R²), C(R²), C(R²)(R^{2a}), C(O), S(O), S(O)₂, S(O)(NR²), S(O)(R²), or P(O)(R²). In some embodiments, W³ is N, N(R³), C(R³), C(R³)(R^{3a}), C(O), S(O), S(O)₂, S(O)(NR³), S(O)(R³), or P(O)(R³). In some embodiments, W⁴ is N, N(R⁴), C(R⁴), C(R⁴)(R^{4a}), C(O), S(O), S(O)₂, S(O)(NR⁴), S(O)(R⁴), or P(O)(R⁴). In some embodiments, W¹ is N, N(R¹), C(R¹), C(R¹)(R^{1a}), or C(O). In some embodiments, W² is N, N(R²), C(R²), C(R²)(R²), or C(O). In some embodiments, W³ is N, N(R³), C(R³), C(R³)(R^{3a}), or C(O). In some embodiments, W⁴ is N, N(R⁴), C(R⁴), C(R⁴)(R^{4a}), or C(O).

In some embodiments, R² is C₁₋₆alkyl, optionally substituted with one, two, or three R^{20b}. In some embodiments, R² is C₁₋₅alkyl, optionally substituted with one, two, or three R^{20b}. In some embodiments, R² is C₁₋₄alkyl, optionally substituted with one, two, or three R^{20b}. In some embodiments, R² is C₁₋₃alkyl, optionally substituted with one, two, or three R^{20b}. In some embodiments, R² is C₁₋₂alkyl, optionally substituted with one, two, or three R^{20b}. In some embodiments, R² is C₁alkyl, optionally substituted with one, two, or three R^{20b}. In some embodiments, R² is C₂alkyl, optionally substituted with one, two, or three R^{20b}. In some embodiments, R² is C₃alkyl, optionally substituted with one, two, or three R^{20b}. In some embodiments, R² is C₄alkyl, optionally substituted with one, two, or three R^{20b}. In some embodiments, R² is C₅alkyl, optionally substituted with one, two, or three R^{20b}. In some embodiments, R² is C₆alkyl, optionally substituted with one, two, or three R^{20b}. In some embodiments, R² is C₁₋₆alkyl. In some embodiments, R² is C₁₋₅alkyl. In some embodiments, R² is C₁₋₄alkyl. In some embodiments, R² is C₁₋₃alkyl. In some embodiments, R² is C₁₋₂alkyl. In some embodiments, R² is C₁alkyl. In some embodiments, R² is C₂alkyl. In some embodiments, R² is C₃alkyl. In some embodiments, R² is C₄alkyl. In some embodiments, R² is C₅alkyl. In some embodiments, R² is C₆alkyl. In some embodiments, each R^{20b} is independently selected from —N(R²²)(R²³), R²² is C₁₋₆alkyl, and R²³ is C₁₋₆alkyl. In some embodiments, R² is C₂₋₆alkynyl, optionally substituted with one, two, or three R^{20b}. In some embodiments, R² is C₂₋₅alkynyl, optionally substituted with one, two, or three R^{20b}In some embodiments, R² is C₂₋₄alkynyl, optionally substituted with one, two, or three R^{20b}. In some embodiments, R² is C₂₋₃alkynyl, optionally substituted with one, two, or three R^{20b}. In some embodiments, R² is C₂alkynyl, optionally substituted with one, two, or three R^{20b}. In some embodiments, R² is C₃alkynyl, optionally substituted with one, two, or three R^{20b}. In some embodiments, R² is C₄alkynyl, optionally substituted with one, two, or three R^{20b}. In some embodiments, R² is C₅alkynyl, optionally substituted with one, two, or three R^{20b}. In some embodiments, R² is C₆alkynyl, optionally substituted with one, two, or three R^{20b}. In some embodiments, R² is C₂₋₆alkynyl. In some embodiments, R² is C₂₋₅alkynyl. In some embodiments, R² is C₂₋₄alkynyl. In some embodiments, R² is C₂₋₃alkynyl. In some embodiments, R² is C₂alkynyl. In some embodiments, R² is C₃alkynyl. In some embodiments, R² is C₄alkynyl. In some embodiments, R² is C₅alkynyl. In some embodiments, R² is C₆alkynyl. In some embodiments, R² is selected from

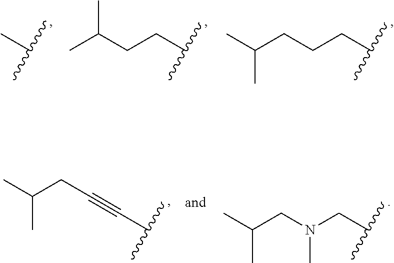

In some embodiments, R² is

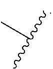

In some embodiments, R² is

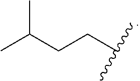 [SMILES: *CCC(C)C]

In some embodiments, R² is

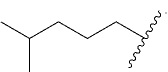 [SMILES: *CCCC(C)C]

In some embodiments, R² is

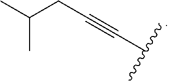 [SMILES: *C#CCC(C)C]

In some embodiments, R² is

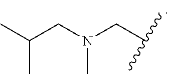 [SMILES: *CN(C)CC(C)C]

In some embodiments, R² is

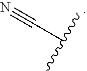 [SMILES: *C#N]

In some embodiments, R² is

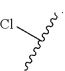 [SMILES: *Cl]

In some embodiments, R² is —OR¹². In some embodiments, R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}. In some embodiments, R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, R² is —OR¹² and R¹² is independently selected from C₁₋₆alkyl, C₃₄cycloalkyl, C₃₋₅heterocycloalkyl, and —CH₂—C₄₋₅heterocycloalkyl, wherein C₁₋₆alkyl, C₃₋₄cycloalkyl, C₃₋₅heterocycloalkyl, and —CH₂—C₄₋₅heterocycloalkyl, are optionally substituted with one, two, or three R^{20k}; and each R^{20k} is independently selected from oxo, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, R² is

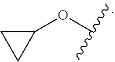 [SMILES: *OC1CC1]

In some embodiments, R² is

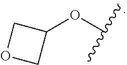 [SMILES: *OC1COC1]

In some embodiments, R² is,

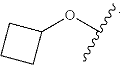 [SMILES: *OC1CCC1]

In some embodiments, R² is

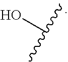 [SMILES: *O]

In some embodiments, R² is

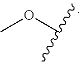 [SMILES: *OC]

In some embodiments, R² is

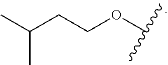 [SMILES: *OCCC(C)C]

In some embodiments, R² is

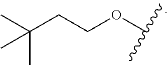 [SMILES: *OCCC(C)(C)C]

In some embodiments, R² is

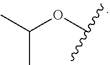 [SMILES: *OC(C)C]

In some embodiments, R² is

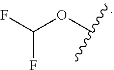 [SMILES: *OC(F)F]

In some embodiments, R² is

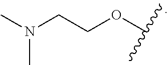 [SMILES: *OCCN(C)C]

In some embodiments, R² is

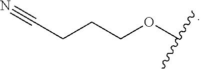 [SMILES: *OCCCC#N]

In some embodiments, R² is

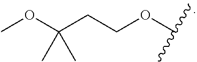 [SMILES: *OCCC(C)(C)OC]

In some embodiments, R² is

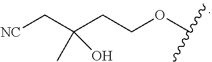 [SMILES: *OCCC(C)(O)CC#N]

In some embodiments, R² is

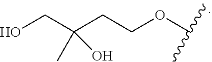 [SMILES: *OCCC(C)(O)CO]

In some embodiments, R² is

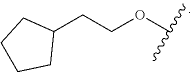 [SMILES: *OCCC1CCCC1]

In some embodiments, R² is

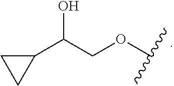 [SMILES: *OCC(O)C1CC1]

In some embodiments, R² is

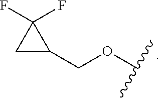 [SMILES: *OCC1CC1(F)F]

In some emobodiments, R² is

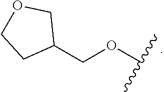 [SMILES: *OCC1CCOC1]

In embodiment, R² is

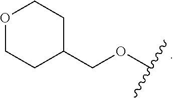 [SMILES: *OCC1CCOCC1]

In some embodiments, R² is

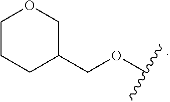 [SMILES: *OCC1CCCOC1]

In some embodiments, R² is C. In some embodiments, R² is

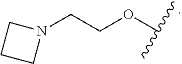 [SMILES: *OCCN1CCC1]

In some embodiments, R² is

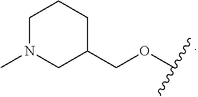 [SMILES: *OCC1CCCN(C)C1]

In some embodiments, R² is

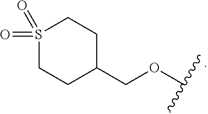 [SMILES: *OCC1CCS(=O)(=O)CC1]

In some embodiments, R² is

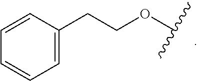 [SMILES: *OCCc1ccccc1]

In some embodiments, R² is

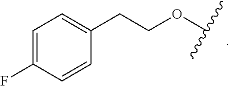 [SMILES: *OCCc1ccc(F)cc1]

In some embodiments, R² is

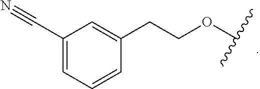 [SMILES: *OCCc1cccc(C#N)c1]

In some embodiments, R² is —N(R¹²)(R¹³). In some embodiments, R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments, R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}; each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, and —S(O)₂R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen; R²¹ is C₁₋₆alkyl; and R²⁵ is C₁₋₆alkyl. In some embodiments, R² is

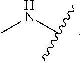 [SMILES: *NC]

In some embodiments, R² is

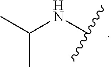 [SMILES: *NC(C)C]

In some embodiments, R² is

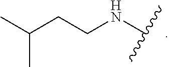 [SMILES: *NCCC(C)C]

In some embodiments, R² is

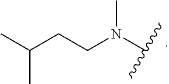 [SMILES: *N(C)CCC(C)C]

In some embodiments, R² is

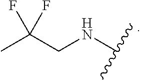 [SMILES: *NCC(C)(F)F]

In some embodiments, R² is

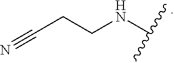 [SMILES: *NCCC#N]

In some embodiments R² is

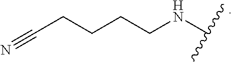 [SMILES: *NCCCCC#N]

In some embodiments, R² is

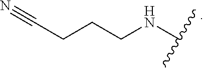 [SMILES: *NCCCC#N]

In some embodiments, R² is

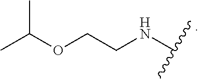 [SMILES: *NCCOC(C)C]

In some embodiments, R² is

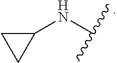 [SMILES: *NC1CC1]

In some embodiments, R² is

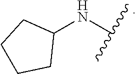 [SMILES: *NC1CCCC1]

In some embodiments, R² is

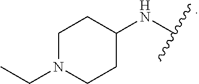 [SMILES: *NC1CCN(CC)CC1]

In some embodiments, R² is

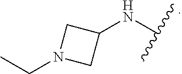 [SMILES: *NC1CN(CC)C1]

In some embodiments, R² is

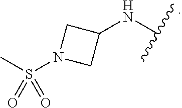 [SMILES: *NC1CN(S(C)(=O)=O)C1]

In some embodiments, R² is

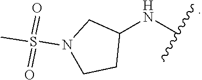 [SMILES: *NC1CCN(S(C)(=O)=O)C1]

In some embodiments, R² is

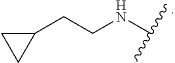 [SMILES: *NCCC1CC1]

In some embodiments, R² is

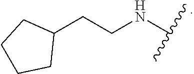 [SMILES: *NCCC1CCCC1]

In some embodiments, R² is

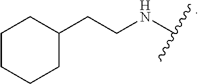 [SMILES: *NCCC1CCCCC1]

In some embodiments, R² is

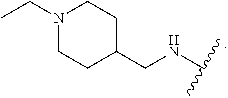 [SMILES: *NCC1CCN(CC)CC1]

In some embodiments, R² is

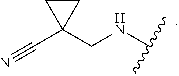 [SMILES: *NCC1(C#N)CC1]

In some embodiments, R² is

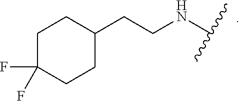 [SMILES: *NCCC1CCC(F)(F)CC1]

In some embodiments, R² is

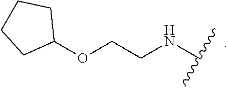 [SMILES: *NCCOC1CCCC1]

In some embodiments, R² is

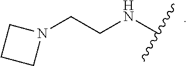 [SMILES: *NCCN1CCC1]

In some embodiments, R² is

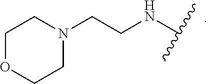 [SMILES: *NCCN1CCOCC1]

In some embodiments, R² is

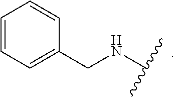 [SMILES: *NCc1ccccc1]

In some embodiments, R² is

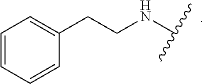 [SMILES: *NCCc1ccccc1]

In some embodiments, R² is

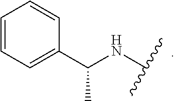 [SMILES: *N[C@H](C)c1ccccc1]

In some embodiments, R² is

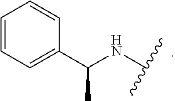 [SMILES: *N[C@@H](C)c1ccccc1]

In some embodiments, R² is

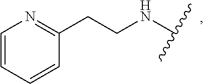 [SMILES: *NCCc1ccccn1]

In some embodiments, R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}. In some embodiments, R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkyl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is selected from C₃₋₆cycloalkyl, C₂₋₉heterocycloalkyl, C₆₋₁₀aryl, and C₁₋₉ heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂; each R²² is independently C₁₋₆alkyl; each R²³ is C₁₋₆alkyl; and each R²⁵ is selected from C₁₋₆alkyl, C₃₋₆cycloalkyl, and —CH₂—C₃₋₆cycloalkyl. In some embodiments, R² is

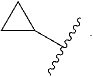 [SMILES: *C1CC1]

In some embodiments, R² is

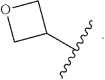 [SMILES: *C1COC1]

In some embodiments, R² is

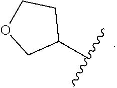 [SMILES: *C1CCOC1]

In some embodiments, R² is

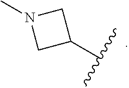 [SMILES: *C1CN(C)C1]

In some embodiments, R² is

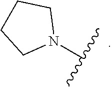 [SMILES: *N1CCCC1]

In some embodiments, R² is

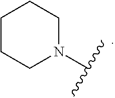 [SMILES: *N1CCCCC1]

In some embodiments, R² is

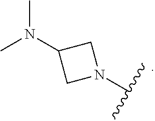 [SMILES: *N1CC(N(C)C)C1]

In some embodiments, R² is

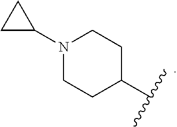 [SMILES: *C1CCN(C2CC2)CC1]

In some embodiments, R² is

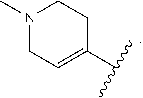 [SMILES: *C1=CCN(C)CC1]

In some embodiments, R² is

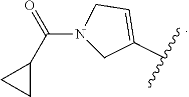 [SMILES: *C1=CCN(C(=O)C2CC2)C1]

In some embodiments, R² is

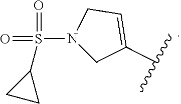 [SMILES: *C1=CCN(S(=O)(=O)C2CC2)C1]

In some embodiments, R² is

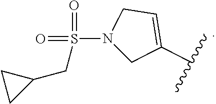 [SMILES: *C1=CCN(S(=O)(=O)CC2CC2)C1]

In some embodiments, R² is

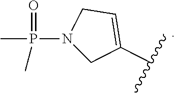 [SMILES: *C1=CCN(P(C)(C)=O)C1]

In some embodiments, R² is

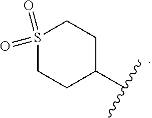 [SMILES: *C1CCS(=O)(=O)CC1]

In some embodiments, R² is

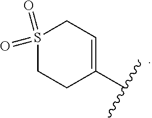 [SMILES: *C1=CCS(=O)(=O)CC1]

In some embodiments, R² is

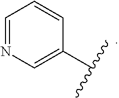 [SMILES: *c1cccnc1]

In some embodiments, R² is

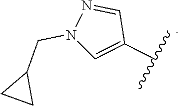 [SMILES: *c1cnn(CC2CC2)c1]

In some embodiments, R² is independently selected from —C(O)OR¹² and —C(O)N(R¹²)(R¹³). In some embodiments, R² is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, R³ is independently selected from —C(O)OR¹² and —C(O)N(R¹²)(R¹³). In some embodiments, R³ is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R³ is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, R² is

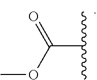 [SMILES: *C(=O)OC]

In some embodiments, R² is

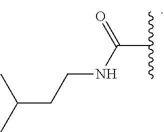 [SMILES: *C(=O)NCCC(C)C]

In some embodiments, R² is

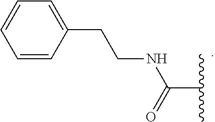 [SMILES: *C(=O)NCCc1ccccc1]

In some embodiments, R³ is

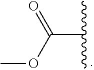 [SMILES: *C(=O)OC]

In some embodiments, R³ is

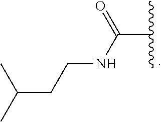 [SMILES: *C(=O)NCCC(C)C]

In some embodiments, R³ is

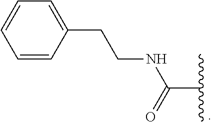 [SMILES: *C(=O)NCCc1ccccc1]

In some embodiments, R² is selected from

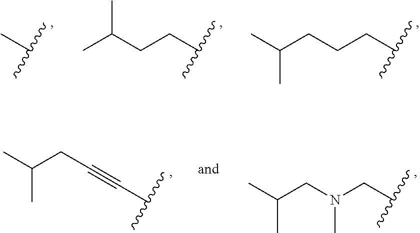

each of which is optionally substituted with one, two, or three R^{20b}. In some embodiments, R² is

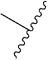

optionally substituted with one, two, or three R^{20b}. In some embodiments, R² is

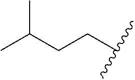 [SMILES: *CCC(C)C]

optionally substituted with one, two, or three R^{20b}. In some embodiments, R² is

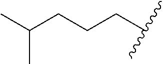 [SMILES: *CCCC(C)C]

optionally substituted with one, two, or three R^{20b}. In some embodiments, R² is

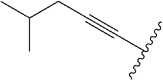 [SMILES: *C#CCC(C)C]

optionally substituted with one, two, or three R^{20b}. In some embodiments, R² is

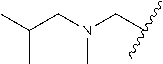 [SMILES: *CN(C)CC(C)C]

optionally substituted with one, two, or three R^{20b}.

In some embodiments, R² is

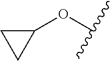 [SMILES: *OC1CC1]

optionally substituted with one, two, or three R^{20k}. In some embodiments, R² is

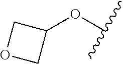 [SMILES: *OC1COC1]

optionally substituted with one, two, or three R^{20k}. In some embodiments, R² is,

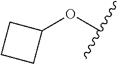 [SMILES: *OC1CCC1]

optionally substituted with one, two, or three R^{20k}. In some embodiments, R² is

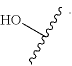 [SMILES: *O]

In some embodiments, R² is

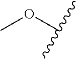 [SMILES: *OC]

optionally substituted with one, two, or three R^{20k}. In some embodiments, R² is

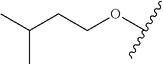 [SMILES: *OCCC(C)C]

optionally substituted with one, two, or three R^{20k}. In some embodiments, R² is

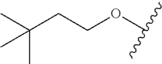 [SMILES: *OCCC(C)(C)C]

optionally substituted with one, two, or three R^{20k}. In some embodiments, R² is

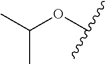 [SMILES: *OC(C)C]

optionally substituted with one, two, or three R^{20k}. In some embodiments, R² is

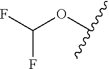 [SMILES: *OC(F)F]

optionally substituted with one R^{k}. In some embodiments, R² is

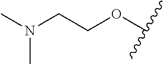 [SMILES: *OCCN(C)C]

optionally substituted with one, two, or three R^{20k}. In some embodiments, R² is

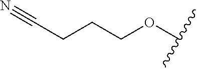 [SMILES: *OCCCC#N]

optionally substituted with one, two, or three R^{20k}. In some embodiments, R² is

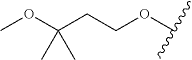 [SMILES: *OCCC(C)(C)OC]

optionally substituted with one, two, or three R^{20k}. In some embodiments, R² is

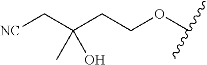 [SMILES: *OCCC(C)(O)CC#N]

optionally substituted with one, two, or three R^{20k}. In some embodiments, R² is

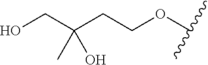 [SMILES: *OCCC(C)(O)CO]

optionally substituted with one, two, or three R^{20k}. In some embodiments, R² is

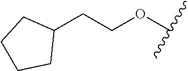 [SMILES: *OCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

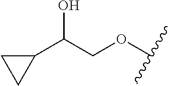 [SMILES: *OCC(O)C1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

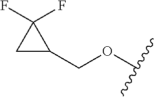 [SMILES: *OCC1CC1(F)F]

optionally substituted with one, two, or three R^{20k}. In some embodiments, R² is

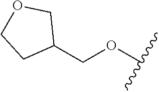 [SMILES: *OCC1CCOC1]

optionally substituted with one, two, or three R^{20k}. In some embodiments, R² is

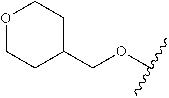 [SMILES: *OCC1CCOCC1]

optionally substituted with one, two, or three R^{20k}. In some embodiments, R² is

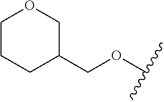 [SMILES: *OCC1CCCOC1]

optionally substituted with one, two, or three R^{20k}. In some embodiments, R² is

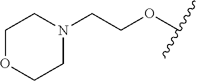 [SMILES: *OCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

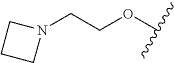 [SMILES: *OCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

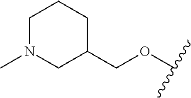 [SMILES: *OCC1CCCN(C)C1]

optionally substituted with one, two, or three R^{20k}. In some embodiments, R² is

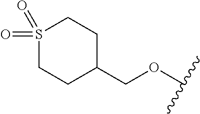 [SMILES: *OCC1CCS(=O)(=O)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

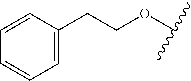 [SMILES: *OCCc1ccccc1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

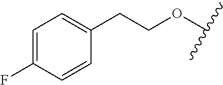 [SMILES: *OCCc1ccc(F)cc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

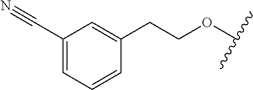 [SMILES: *OCCc1cccc(C#N)c1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is

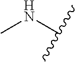 [SMILES: *NC]

optionally substituted with one, two, or three R^{20k}. In some embodiments, R² is

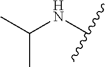 [SMILES: *NC(C)C]

optionally substituted with one, two, or three R^{20k}. In some embodiments, R²

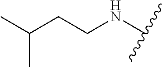 [SMILES: *NCCC(C)C]

is optionally substituted with one, two, or three R^{20k}. In some embodiments, R² is

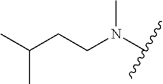 [SMILES: *N(C)CCC(C)C]

optionally substituted with one, two, or three R^{20k}. In some embodiments, R² is

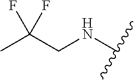 [SMILES: *NCC(C)(F)F]

optionally substituted with one, two, or three R^{20k}. In some embodiments, R² is

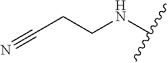 [SMILES: *NCCC#N]

optionally substituted with one, two, or three R^{20k}. In some embodiments, R² is

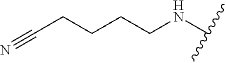 [SMILES: *NCCCCC#N]

optionally substituted with one, two, or three R^{20k}. In some embodiments, R² is

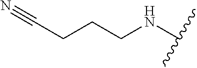 [SMILES: *NCCCC#N]

optionally substituted with one, two, or three R^{20k}. In some embodiments, R² is

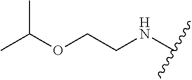 [SMILES: *NCCOC(C)C]

optionally substituted with one, two, or three R^{20k}. In some embodiments, R² is

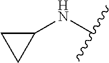 [SMILES: *NC1CC1]

optionally substituted with one, two, or three R^{20k}. In some embodiments, R² is

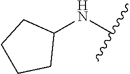 [SMILES: *NC1CCCC1]

optionally substituted with one, two, or three R^{20k}. In some embodiments, R² is

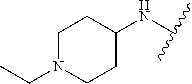 [SMILES: *NC1CCN(CC)CC1]

optionally substituted with one, two, or three R^{20k}. In some embodiments, R² is

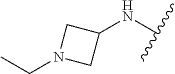 [SMILES: *NC1CN(CC)C1]

optionally substituted with one, two, or three R^{20k}. In some embodiments, R² is

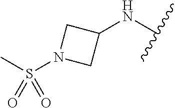 [SMILES: *NC1CN(S(C)(=O)=O)C1]

optionally substituted with one, two, or three R^{20k}. In some embodiments, R² is

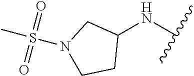 [SMILES: *NC1CCN(S(C)(=O)=O)C1]

optionally substituted with one, two, or three R^{20k}. In some embodiments, R² is

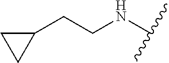 [SMILES: *NCCC1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

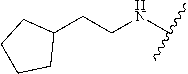 [SMILES: *NCCC1CCCC1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R⁴)S(O)₂R²⁵, —C(O)R²⁵, —S(O)₂R²⁵, —S(O)₂N(R²²)(R²³), and —OC(O)R²⁵. In some embodiments, R² is

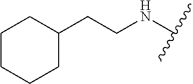 [SMILES: *NCCC1CCCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

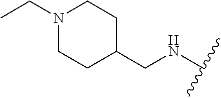 [SMILES: *NCC1CCN(CC)CC1]

optionally substituted with one, two, or three R^{20k}. In some embodiments, R² is

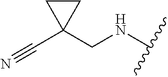 [SMILES: *NCC1(C#N)CC1]

optionally substituted with one, two, or three R^{20k}. In some embodiments, R² is

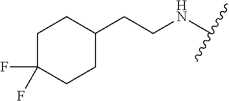 [SMILES: *NCCC1CCC(F)(F)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

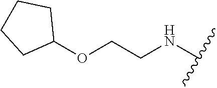 [SMILES: *NCCOC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

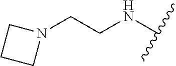 [SMILES: *NCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

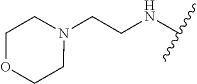 [SMILES: *NCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

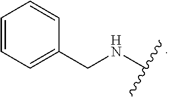 [SMILES: *NCc1ccccc1]

In some embodiments, R² is

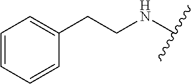 [SMILES: *NCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

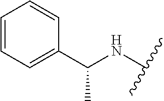 [SMILES: *N[C@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

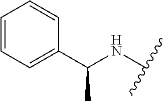 [SMILES: *N[C@@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

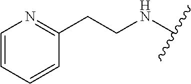 [SMILES: *NCCc1ccccn1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is

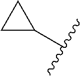 [SMILES: *C1CC1]

optionally substituted with one, two, or three R^{20b}. In some embodiments, R² is

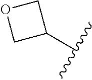 [SMILES: *C1COC1]

optionally substituted with one, two, or three R^{20b}. In some embodiments, R² is

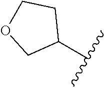 [SMILES: *C1CCOC1]

optionally substituted with one, two, or three R^{20b}. In some embodiments, R² is

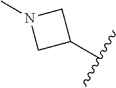 [SMILES: *C1CN(C)C1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

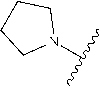 [SMILES: *N1CCCC1]

optionally substituted with one, two, or three R^{20b}. In some embodiments, R² is

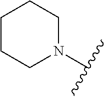 [SMILES: *N1CCCCC1]

optionally substituted with one, two, or three R^{20b}. In some embodiments, R² is

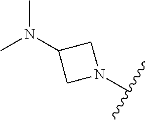 [SMILES: *N1CC(N(C)C)C1]

optionally substituted with one, two, or three R^{20b}. In some embodiments, R² is

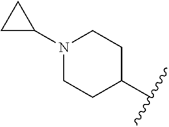 [SMILES: *C1CCN(C2CC2)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R² is

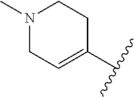 [SMILES: *C1=CCN(C)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R² is

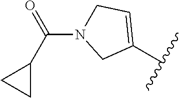 [SMILES: *C1=CCN(C(=O)C2CC2)C1]

optionally substituted with one, two, or three R^{20b}. In some embodiments, R² is

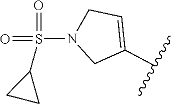 [SMILES: *C1=CCN(S(=O)(=O)C2CC2)C1]

optionally substituted with one, two, or three R^{20b}. In some embodiments, R² is

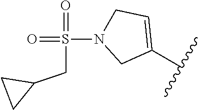 [SMILES: *C1=CCN(S(=O)(=O)CC2CC2)C1]

optionally substituted with one, two, or three R^{20b}. In some embodiments, R² is

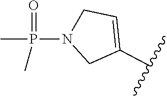 [SMILES: *C1=CCN(P(C)(C)=O)C1]

optionally substituted with one, two, or three R^{20b}. In some embodiments, R² is

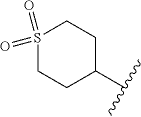 [SMILES: *C1CCS(=O)(=O)CC1]

optionally substituted with one, two, or three R^{20b}. In some embodiments, R² is

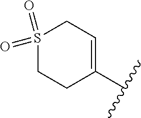 [SMILES: *C1=CCS(=O)(=O)CC1]

optionally substituted with one, two, or three R^{20b}. In some embodiments, R² is

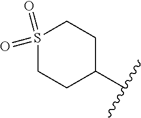 [SMILES: *C1CCS(=O)(=O)CC1]

optionally substituted with one, two, or three R^{20b}. In some embodiments, R² is

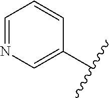 [SMILES: *c1cccnc1]

optionally substituted with one, two, or three R^{20b}. In some embodiments, R² is

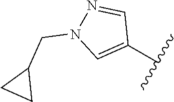 [SMILES: *c1cnn(CC2CC2)c1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

some embodiments, R² is

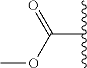 [SMILES: *C(=O)OC]

optionally substituted with one, two, or three R^{20k}. In some embodiments, R² is

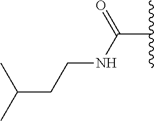 [SMILES: *C(=O)NCCC(C)C]

optionally substituted with one, two, or three R^{20k}. In some embodiments, R² is

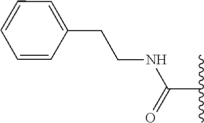 [SMILES: *C(=O)NCCc1ccccc1]

optionally substituted with one, two, or three R^{20k}. In some embodiments, R³ is

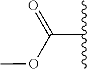 [SMILES: *C(=O)OC]

optionally substituted with one, two, or three R^{20k}. In some embodiments, R³ is

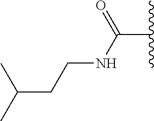 [SMILES: *C(=O)NCCC(C)C]

optionally substituted with one, two, or three R^{20k}. In some embodiments, R³ is

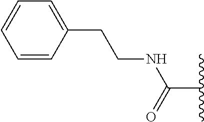 [SMILES: *C(=O)NCCc1ccccc1]

optionally substituted with one, two, or three R^{20k}.

TABLE 1
selected compound formula

| | A |
|---|---|
| 1 | 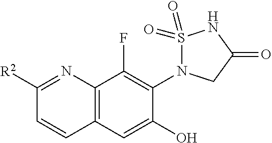 |
| 2 | 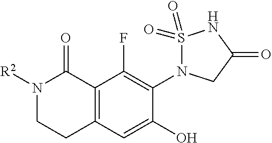 |
| 3 | 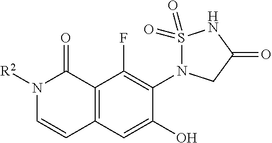 |
| 4 | 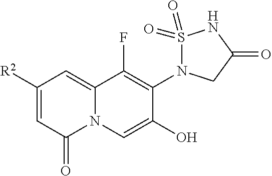 |
| 5 | 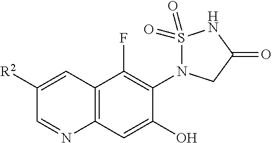 |
| 6 | 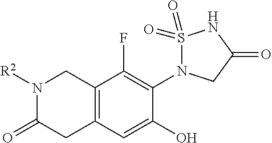 |
| 7 | 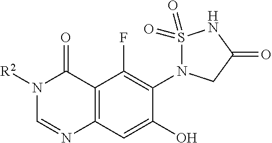 |
| 8 | 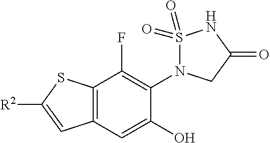 |
| 9 | 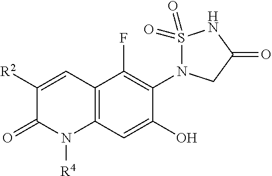 |
| 10 | 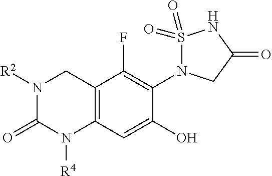 |
| 11 | 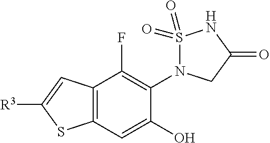 |

TABLE 2
selected R² or R³ embodiments

| | B |
|---|---|
| 1 | 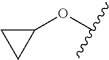 [SMILES: *OC1CC1] |
| 2 | 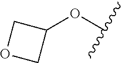 [SMILES: *OC1COC1] |
| 3 | 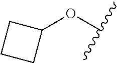 [SMILES: *OC1CCC1] |
| 4 | 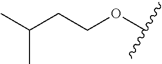 [SMILES: *OCCC(C)C] |
| 5 | 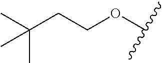 [SMILES: *OCCC(C)(C)C] |
| 6 | 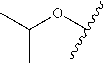 [SMILES: *OC(C)C] |
| 7 | 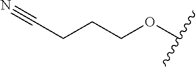 [SMILES: *OCCCC#N] |
| 8 | 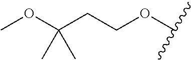 [SMILES: *OCCC(C)(C)OC] |
| 9 | 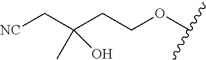 [SMILES: *OCCC(C)(O)CC#N] |
| 10 | 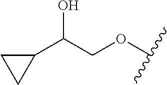 [SMILES: *OCC(O)C1CC1] |
| 11 | 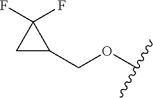 [SMILES: *OCC1CC1(F)F] |
| 12 | 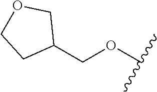 [SMILES: *OCC1CCOC1] |
| 13 | 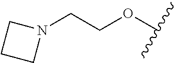 [SMILES: *OCCN1CCC1] |
| 14 | 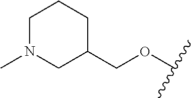 [SMILES: *OCC1CCCN(C)C1] |
| 15 | 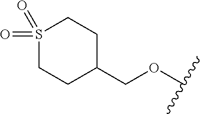 [SMILES: *OCC1CCS(=O)(=O)CC1] |
| 16 | 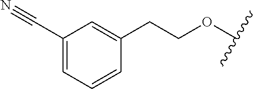 [SMILES: *OCCc1cccc(C#N)c1] |
| 17 | 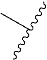 |
| 18 | 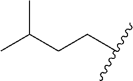 [SMILES: *CCC(C)C] |
| 19 | 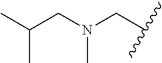 [SMILES: *CN(C)CC(C)C] |
| 20 | 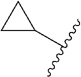 [SMILES: *C1CC1] |
| 21 | 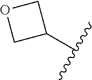 [SMILES: *C1COC1] |
| 22 | 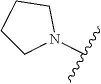 [SMILES: *N1CCCC1] |
| 23 | 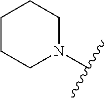 [SMILES: *N1CCCCC1] |
| 24 | 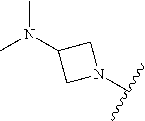 [SMILES: *N1CC(N(C)C)C1] |
| 25 | 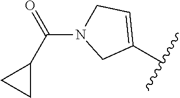 [SMILES: *C1=CCN(C(=O)C2CC2)C1] |
| 26 | 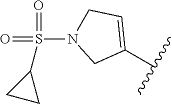 [SMILES: *C1=CCN(S(=O)(=O)C2CC2)C1] |
| 27 | 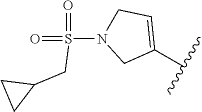 [SMILES: *C1=CCN(S(=O)(=O)CC2CC2)C1] |
| 28 | 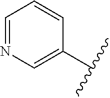 [SMILES: *c1cccnc1] |
| 29 | 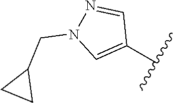 [SMILES: *c1cnn(CC2CC2)c1] |
| 30 | 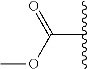 [SMILES: *C(=O)OC] |
| 31 | 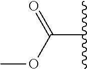 [SMILES: *C(=O)OC] |
| 32 | 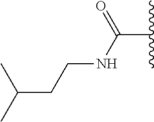 [SMILES: *C(=O)NCCC(C)C] |
| 33 | 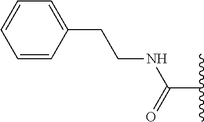 [SMILES: *C(=O)NCCc1ccccc1] |
| 34 | 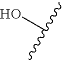 [SMILES: *O] |
| 35 | 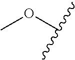 [SMILES: *OC] |
| 36 | 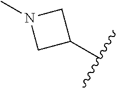 [SMILES: *C1CN(C)C1] |
| 37 | 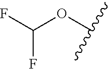 [SMILES: *OC(F)F] |
| 38 | 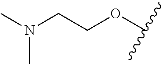 [SMILES: *OCCN(C)C] |
| 39 | 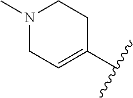 [SMILES: *C1=CCN(C)CC1] |
| 40 | 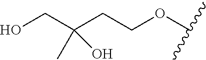 [SMILES: *OCCC(C)(O)CO] |
| 41 | 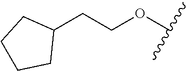 [SMILES: *OCCC1CCCC1] |
| 42 | 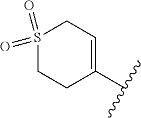 [SMILES: *C1=CCS(=O)(=O)CC1] |
| 43 | 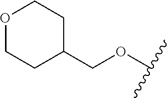 [SMILES: *OCC1CCOCC1] |
| 44 | 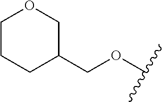 [SMILES: *OCC1CCCOC1] |
| 45 | 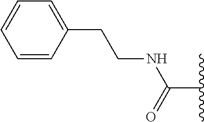 [SMILES: *C(=O)NCCc1ccccc1] |
| 46 | 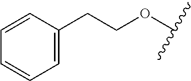 [SMILES: *OCCc1ccccc1] |
| 47 | 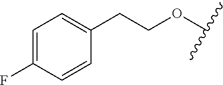 [SMILES: *OCCc1ccc(F)cc1] |
| 48 | 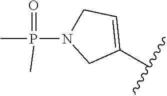 [SMILES: *C1=CCN(P(C)(C)=O)C1] |
| 49 | 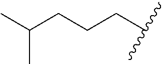 [SMILES: *CCCC(C)C] |
| 50 | 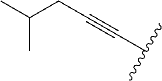 [SMILES: *C#CCC(C)C] |
| 51 | 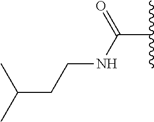 [SMILES: *C(=O)NCCC(C)C] |
| 52 | 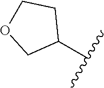 [SMILES: *C1CCOC1] |
| 53 | 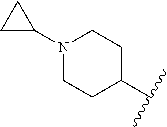 [SMILES: *C1CCN(C2CC2)CC1] |
| 54 | 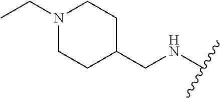 [SMILES: *NCC1CCN(CC)CC1] |
| 55 | 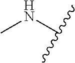 [SMILES: *NC] |
| 56 | 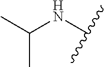 [SMILES: *NC(C)C] |
| 57 | 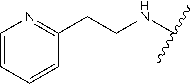 [SMILES: *NCCc1ccccn1] |
| 58 | 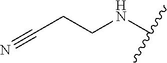 [SMILES: *NCCC#N] |
| 59 | 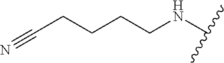 [SMILES: *NCCCCC#N] |
| 60 | 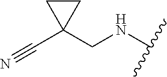 [SMILES: *NCC1(C#N)CC1] |
| 61 | 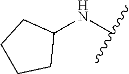 [SMILES: *NC1CCCC1] |
| 62 | 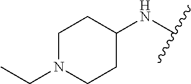 [SMILES: *NC1CCN(CC)CC1] |
| 63 | 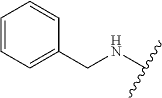 [SMILES: *NCc1ccccc1] |
| 64 | 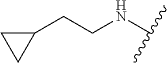 [SMILES: *NCCC1CC1] |
| 65 | 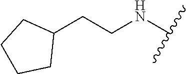 [SMILES: *NCCC1CCCC1] |
| 66 | 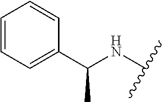 [SMILES: *N[C@@H](C)c1ccccc1] |
| 67 | 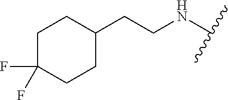 [SMILES: *NCCC1CCC(F)(F)CC1] |
| 68 | 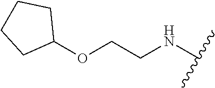 [SMILES: *NCCOC1CCCC1] |
| 69 | 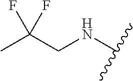 [SMILES: *NCC(C)(F)F] |
| 70 | 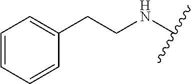 [SMILES: *NCCc1ccccc1] |
| 71 | 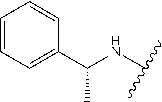 [SMILES: *N[C@H](C)c1ccccc1] |
| 72 | 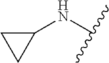 [SMILES: *NC1CC1] |
| 73 | 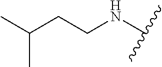 [SMILES: *NCCC(C)C] |
| 74 | 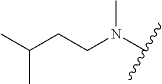 [SMILES: *N(C)CCC(C)C] |
| 75 | 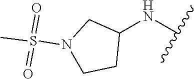 [SMILES: *NC1CCN(S(C)(=O)=O)C1] |
| 76 | 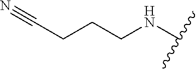 [SMILES: *NCCCC#N] |
| 77 | 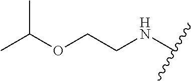 [SMILES: *NCCOC(C)C] |
| 78 | 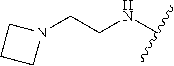 [SMILES: *NCCN1CCC1] |
| 79 | 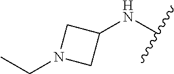 [SMILES: *NC1CN(CC)C1] |
| 80 | 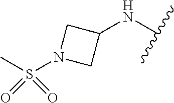 [SMILES: *NC1CN(S(C)(=O)=O)C1] |
| 81 | 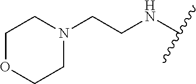 [SMILES: *NCCN1CCOCC1] |
| 82 | 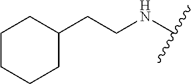 [SMILES: *NCCC1CCCCC1] |
| 83 | 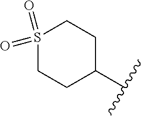 [SMILES: *C1CCS(=O)(=O)CC1] |

TABLE 3
selected R⁴ embodiments

| | C |
|---|---|
| 1 | H |
| 2 | CH₂CH₃ |
| 3 | Cyclopropyl |
| 4 | CH₃ |
| 5 | CN |
| 6 | Cyclobutyl |

In embodiments, the subject compound is a compound having a formula comprising a combination of a cell of Table 1 and a cell of Table 2. For example, a compound may have the formula of cell A1 of Table 1 and the R² of cell B1 of Table 2 and would be described by the formula “A1B1”. For example, a compound may have the formula of cell A11 of Table 1 and the R³ of cell B1 of Table 2 and would be described by the formula “A11B1”. For example, a compound may have the formula of cell A9 of Table 1 and the R² of cell B1 of Table 2 and R⁴ of cell C4 of Table 3 and would be described by the formula “A9B1C4”. In embodiments, the subject compound has a formula selected from A1B1, A1B2, A1B3, A1B4, A1B5, A1B6, A1B7, A1B8, A1B9, A1B10, A1B11, A1B12, A1B13, A1B14, A1B15, A1B16, A1B17, A1B18, A1B19, A1B20, A1B21, A1B22, A1B23, A1B24, A1B25, A1B26, A1B27, A1B28, A1B29, A1B30, A1B31, A1B32, A1B33, A1B34, A1B35, A1B36, A1B37, A1B38, A1B39, A1B40, A1B41, A1B42, A1B43, A1B44, A1B45, A1B46, A1B47, A1B48, A1B49, A1B50, A1B51, A1B52, A1B53, A1B83, A1B54, A1B55, A1B56, A1B57, A1B58, A1B59, A1B60, A1B61, A1B62, A1B63, A1B64, A1B65, A1B66, A1B67, A1B68, A1B69, A1B70, A1B71, A1B72, A1B73, A1B74, A1B75, A1B76, A1B77, A1B78, A1B79, A1B80, A1B81, A1B82, A2B1, A2B2, A2B3, A2B4, A2B5, A2B6, A2B7, A2B8, A2B9, A2B10, A2B11, A2B12, A2B13, A2B14, A2B15, A2B16, A2B17, A2B18, A2B19, A2B20, A2B21, A2B22, A2B23, A2B24, A2B25, A2B26, A2B27, A2B28, A2B29, A2B30, A2B31, A2B32, A2B33, A2B34, A2B35, A2B36, A2B37, A2B38, A2B39, A2B40, A2B41, A2B42, A2B43, A2B44, A2B45, A2B46, A2B47, A2B48, A2B49, A2B50, A2B51, A2B52, A2B53, A2B83, A3B1, A3B2, A3B3, A3B4, A3B5, A3B6, A3B7, A3B8, A3B9, A3B10, A3B11, A3B12, A3B13, A3B14, A3B15, A3B16, A3B17, A3B18, A3B19, A3B20, A3B21, A3B22, A3B23, A3B24, A3B25, A3B26, A3B27, A3B28, A3B29, A3B30, A3B31, A3B32, A3B33, A3B34, A3B35, A3B36, A3B37, A3B38, A3B39, A3B40, A3B41, A3B42, A3B43, A3B44, A3B45, A3B46, A3B47, A3B48, A3B49, A3B50, A3B51, A3B52, A3B53, A3B83, A4B1, A4B2, A4B3, A4B4, A4B5, A4B6, A4B7, A4B8, A4B9, A4B10, A4B11, A4B12, A4B13, A4B14, A4B15, A4B16, A4B17, A4B18, A4B19, A4B20, A4B21, A4B22, A4B23, A4B24, A4B25, A4B26, A4B27, A4B28, A4B29, A4B30, A4B31, A4B32, A4B33, A4B34, A4B35, A4B36, A4B37, A4B38, A4B39, A4B40, A4B41, A4B42, A4B43, A4B44, A4B45, A4B46, A4B47, A4B48, A4B49, A4B50, A4B51, A4B52, A4B53, A4B83, A4B54, A4B55, A4B56, A4B57, A4B58, A4B59, A4B60, A4B61, A4B62, A4B63, A4B64, A4B65, A4B66, A4B67, A4B68, A4B69, A4B70, A4B71, A4B72, A4B73, A4B74, A4B75, A4B76, A4B77, A4B78, A4B79, A4B80, A4B81, A4B82, A5B1, A5B2, A5B3, A5B4, A5B5, A5B6, A5B7, A5B8, A5B9, A5B10, A5B11, A5B12, A5B13, A5B14, A5B15, A5B16, A5B17, A5B18, A5B19, A5B20, A5B21, A5B22, A5B23, A5B24, A5B25, A5B26, A5B27, A5B28, A5B29, A5B30, A5B31, A5B32, A5B33, A5B34, A5B35, A5B36, A5B37, A5B38, A5B39, A5B40, A5B41, A5B42, A5B43, A5B44, A5B45, A5B46, A5B47, A5B48, A5B49, A5B50, A5B51, A5B52, A5B53, A5B83, A5B54, A5B55, A5B56, A5B57, A5B58, A5B59, A5B60, A5B61, A5B62, A5B63, A5B64, A5B65, A5B66, A5B67, A5B68, A5B69, A5B70, A5B71, A5B72, A5B73, A5B74, A5B75, A5B76, A5B77, A5B78, A5B79, A5B80, A5B81, A5B82, A6B1, A6B2, A6B3, A6B4, A6B5, A6B6, A6B7, A6B8, A6B9, A6B10, A6B11, A6B12, A6B13, A6B14, A6B15, A6B16, A6B17, A6B18, A6B19, A6B20, A6B21, A6B22, A6B23, A6B24, A6B25, A6B26, A6B27, A6B28, A6B29, A6B30, A6B31, A6B32, A6B33, A6B34, A6B35, A6B36, A6B37, A6B38, A6B39, A6B40, A6B41, A6B42, A6B43, A6B44, A6B45, A6B46, A6B47, A6B48, A6B49, A6B50, A6B51, A6B52, A6B53, A6B83,A7B1, A7B2, A7B3, A7B4, A7B5, A7B6, A7B7, A7B8, A7B9, A7B10, A7B11, A7B12, A7B13, A7B14, A7B15, A7B16, A7B17, A7B18, A7B19, A7B20, A7B21, A7B22, A7B23, A7B24, A7B25, A7B26, A7B27, A7B28, A7B29, A7B30, A7B31, A7B32, A7B33, A7B34, A7B35, A7B36, A7B37, A7B38, A7B39, A7B40, A7B41, A7B42, A7B43, A7B44, A7B45, A7B46, A7B47, A7B48, A7B49, A7B50, A7B51, A7B52, A7B53, A7B83, A8B1, A8B2, A8B3, A8B4, A8B5, A8B6, A8B7, A8B8, A8B9, A8B10, A8B11, A8B12, A8B13, A8B14, A8B15, A8B16, A8B17, A8B18, A8B19, A8B20, A8B21, A8B22, A8B23, A8B24, A8B25, A8B26, A8B27, A8B28, A8B29, A8B30, A8B31, A8B32, A8B33, A8B34, A8B35, A8B36, A8B37, A8B38, A8B39, A8B40, A8B41, A8B42, A8B43, A8B44, A8B45, A8B46, A8B47, A8B48, A8B49, A8B50, A8B51, A8B52, A8B53, A8B83, A8B54, A8B55, A8B56, A8B57, A8B58, A8B59, A8B60, A8B61, A8B62, A8B63, A8B64, A8B65, A8B66, A8B67, A8B68, A8B69, A8B70, A8B71, A8B72, A8B73, A8B74, A8B75, A8B76, A8B77, A8B78, A8B79, A8B80, A8B81, A8B82, A9B1C1, A9B2C1, A9B3C1, A9B4C1, A9B5C1, A9B6C1, A9B7C1, A9B8C1, A9B9C1, A9B10C1, A9B11C1, A9B12C1, A9B13C1, A9B14C1, A9B15C1, A9B16C1, A9B17C1, A9B18C1, A9B19C1, A9B20C1, A9B21C1, A9B22C1, A9B23C1, A9B24C1, A9B25C1, A9B26C1, A9B27C1, A9B28C1, A9B29C1, A9B30C1, A9B31C1, A9B32C1, A9B33C1, A9B34C1, A9B35C1, A9B36C1, A9B37C1, A9B38C1, A9B39C1, A9B40C1, A9B41C1, A9B42C1, A9B43C1, A9B44C1, A9B45C1, A9B46C1, A9B47C1, A9B48C1, A9B49C1, A9B50C1, A9B51C1, A9B52C1, A9B53C1, A9B83C1,A9B54C1, A9B55C1, A9B56C1, A9B57C1, A9B58C1, A9B59C1, A9B60C1, A9B61C1, A9B62C1, A9B63C1, A9B64C1, A9B65C1, A9B66C1, A9B67C1, A9B68C1, A9B69C1, A9B70C1, A9B71C1, A9B72C1, A9B73C1, A9B74C1, A9B75C1, A9B76C1, A9B77C1, A9B78C1, A9B79C1, A9B80C1, A9B81C1, A9B82C1, A9B1C2, A9B2C2, A9B3C2, A9B4C2, A9B5C2, A9B6C2, A9B7C2, A9B8C2, A9B9C2, A9B10C2, A9B11C2, A9B12C2, A9B13C2, A9B14C2, A9B15C2, A9B16C2, A9B17C2, A9B18C2, A9B19C2, A9B20C2, A9B21C2, A9B22C2, A9B23C2, A9B24C2, A9B25C2, A9B26C2, A9B27C2, A9B28C2, A9B29C2, A9B30C2, A9B31C2, A9B32C2, A9B33C2, A9B34C2, A9B35C2, A9B36C2, A9B37C2, A9B38C2, A9B39C2, A9B40C2, A9B41C2, A9B42C2, A9B43C2, A9B44C2, A9B45C2, A9B46C2, A9B47C2, A9B48C2, A9B49C2, A9B50C2, A9B51C2, A9B52C2, A9B53C2, A9B83C2,A9B54C2, A9B55C2, A9B56C2, A9B57C2, A9B58C2, A9B59C2, A9B60C2, A9B61C2, A9B62C2, A9B63C2, A9B64C2, A9B65C2, A9B66C2, A9B67C2, A9B68C2, A9B69C2, A9B70C2, A9B71C2, A9B72C2, A9B73C2, A9B74C2, A9B75C2, A9B76C2, A9B77C2, A9B78C2, A9B79C2, A9B80C2, A9B81C2, A9B82C2, A9B1C3, A9B2C3, A9B3C3, A9B4C3, A9B5C3, A9B6C3, A9B7C3, A9B8C3, A9B9C3, A9B10C3, A9B11C3, A9B12C3, A9B13C3, A9B14C3, A9B15C3, A9B16C3, A9B17C3, A9B18C3, A9B19C3, A9B20C3, A9B21C3, A9B22C3, A9B23C3, A9B24C3, A9B25C3, A9B26C3, A9B27C3, A9B28C3, A9B29C3, A9B30C3, A9B31C3, A9B32C3, A9B33C3, A9B34C3, A9B35C3, A9B36C3, A9B37C3, A9B38C3, A9B39C3, A9B40C3, A9B41C3, A9B42C3, A9B43C3, A9B44C3, A9B45C3, A9B46C3, A9B47C3, A9B48C3, A9B49C3, A9B50C3, A9B51C3, A9B52C3, A9B53C3, A9B83C3, A9B54C3, A9B55C3, A9B56C3, A9B57C3, A9B58C3, A9B59C3, A9B60C3, A9B61C3, A9B62C3, A9B63C3, A9B64C3, A9B65C3, A9B66C3, A9B67C3, A9B68C3, A9B69C3, A9B70C3, A9B71C3, A9B72C3, A9B73C3, A9B74C3, A9B75C3, A9B76C3, A9B77C3, A9B78C3, A9B79C3, A9B80C3, A9B81C3, A9B82C3, A9B1C4, A9B2C4, A9B3C4, A9B4C4, A9B5C4, A9B6C4, A9B7C4, A9B8C4, A9B9C4, A9B10C4, A9B11C4, A9B12C4, A9B13C4, A9B14C4, A9B15C4, A9B16C4, A9B17C4, A9B18C4, A9B19C4, A9B20C4, A9B21C4, A9B22C4, A9B23C4, A9B24C4, A9B25C4, A9B26C4, A9B27C4, A9B28C4, A9B29C4, A9B30C4, A9B31C4, A9B32C4, A9B33C4, A9B34C4, A9B35C4, A9B36C4, A9B37C4, A9B38C4, A9B39C4, A9B40C4, A9B41C4, A9B42C4, A9B43C4, A9B44C4, A9B45C4, A9B46C4, A9B47C4, A9B48C4, A9B49C4, A9B50C4, A9B51C4, A9B52C4, A9B53C4, A9B83C4,A9B54C4, A9B55C4, A9B56C4, A9B57C4, A9B58C4, A9B59C4, A9B60C4, A9B61C4, A9B62C4, A9B63C4, A9B64C4, A9B65C4, A9B66C4, A9B67C4, A9B68C4, A9B69C4, A9B70C4, A9B71C4, A9B72C4, A9B73C4, A9B74C4, A9B75C4, A9B76C4, A9B77C4, A9B78C4, A9B79C4, A9B80C4, A9B81C4, A9B82C4, A9B1C5, A9B2C5, A9B3C5, A9B4C5, A9B5C5, A9B6C5, A9B7C5, A9B8C5, A9B9C5, A9B10C5, A9B11C5, A9B12C5, A9B13C5, A9B14C5, A9B15C5, A9B16C5, A9B17C5, A9B18C5, A9B19C5, A9B20C5, A9B21C5, A9B22C5, A9B23C5, A9B24C5, A9B25C5, A9B26C5, A9B27C5, A9B28C5, A9B29C5, A9B30C5, A9B31C5, A9B32C5, A9B33C5, A9B34C5, A9B35C5, A9B36C5, A9B37C5, A9B38C5, A9B39C5, A9B40C5, A9B41C5, A9B42C5, A9B43C5, A9B44C5, A9B45C5, A9B46C5, A9B47C5, A9B48C5, A9B49C5, A9B50C5, A9B51C5, A9B52C5, A9B53C5, A9B83C5, A9B54C5, A9B55C5, A9B56C5, A9B57C5, A9B58C5, A9B59C5, A9B60C5, A9B61C5, A9B62C5, A9B63C5, A9B64C5, A9B65C5, A9B66C5, A9B67C5, A9B68C5, A9B69C5, A9B70C5, A9B71C5, A9B72C5, A9B73C5, A9B74C5, A9B75C5, A9B76C5, A9B77C5, A9B78C5, A9B79C5, A9B80C5, A9B81C5, A9B82C5, A9B1C6, A9B2C6, A9B3C6, A9B4C6, A9B5C6, A9B6C6, A9B7C6, A9B8C6, A9B9C6, A9B10C6, A9B11C6, A9B12C6, A9B13C6, A9B14C6, A9B15C6, A9B16C6, A9B17C6, A9B18C6, A9B19C6, A9B20C6, A9B21C6, A9B22C6, A9B23C6, A9B24C6, A9B25C6, A9B26C6, A9B27C6, A9B28C6, A9B29C6, A9B30C6, A9B31C6, A9B32C6, A9B33C6, A9B34C6, A9B35C6, A9B36C6, A9B37C6, A9B38C6, A9B39C6, A9B40C6, A9B41C6, A9B42C6, A9B43C6, A9B44C6, A9B45C6, A9B46C6, A9B47C6, A9B48C6, A9B49C6, A9B50C6, A9B51C6, A9B52C6, A9B53C6, A9B83C6, A9B54C6, A9B55C6, A9B56C6, A9B57C6, A9B58C6, A9B59C6, A9B60C6, A9B61C6, A9B62C6, A9B63C6, A9B64C6, A9B65C6, A9B66C6, A9B67C6, A9B68C6, A9B69C6, A9B70C6, A9B71C6, A9B72C6, A9B73C6, A9B74C6, A9B75C6, A9B76C6, A9B77C6, A9B78C6, A9B79C6, A9B80C6, A9B81C6, A9B82C6, A10B1C1, A10B2C1, A10B3C1, A10B4C1, A10B5C1, A10B6C1, A10B7C1, A10B8C1, A10B9C1, A10B10C1, A10B11C1, A10B12C1, A10B13C1, A10B14C1, A10B15C1, A10B16C1, A10B17C1, A10B18C1, A10B19C1, A10B20C1, A10B21C1, A10B22C1, A10B23C1, A10B24C1, A10B25C1, A10B26C1, A10B27C1, A10B28C1, A10B29C1, A10B30C1, A10B31C1, A10B32C1, A10B33C1, A10B34C1, A10B35C1, A10B36C1, A10B37C1, A10B38C1, A10B39C1, A10B40C1, A10B41C1, A10B42C1, A10B43C1, A10B44C1, A10B45C1, A10B46C1, A10B47C1, A10B48C1, A10B49C1, A10B50C1, A10B51C1, A10B52C1, A10B53C1, A10B83C1, A10B1C2, A10B2C2, A10B3C2, A10B4C2, A10B5C2, A10B6C2, A10B7C2, A10B8C2, A10B9C2, A10B10C2, A10B11C2, A10B12C2, A10B13C2, A10B14C2, A10B15C2, A10B16C2, A10B17C2, A10B18C2, A10B19C2, A10B20C2, A10B21C2, A10B22C2, A10B23C2, A10B24C2, A10B25C2, A10B26C2, A10B27C2, A10B28C2, A10B29C2, A10B30C2, A10B31C2, A10B32C2, A10B33C2, A10B34C2, A10B35C2, A10B36C2, A10B37C2, A10B38C2, A10B39C2, A10B40C2, A10B41C2, A10B42C2, A10B43C2, A10B44C2, A10B45C2, A10B46C2, A10B47C2, A10B48C2, A10B49C2, A10B50C2, A10B51C2, A10B52C2, A10B53C2, A10B83C2, A10B1C3, A10B2C3, A10B3C3, A10B4C3, A10B5C3, A10B6C3, A10B7C3, A10B8C3, A10B9C3, A10B10C3, A10B11C3, A10B12C3, A10B13C3, A10B14C3, A10B15C3, A10B16C3, A10B17C3, A10B18C3, A10B19C3, A10B20C3, A10B21C3, A10B22C3, A10B23C3, A10B24C3, A10B25C3, A10B26C3, A10B27C3, A10B28C3, A10B29C3, A10B30C3, A10B31C3, A10B32C3, A10B33C3, A10B34C3, A10B35C3, A10B36C3, A10B37C3, A10B38C3, A10B39C3, A10B40C3, A10B41C3, A10B42C3, A10B43C3, A10B44C3, A10B45C3, A10B46C3, A10B47C3, A10B48C3, A10B49C3, A10B50C3, A10B51C3, A10B52C3, A10B53C3, A10B83C3,A10B1C4, A10B2C4, A10B3C4, A10B4C4, A10B5C4, A10B6C4, A10B7C4, A10B8C4, A10B9C4, A10B10C4, A10B11C4, A10B12C4, A10B13C4, A10B14C4, A10B15C4, A10B16C4, A10B17C4, A10B18C4, A10B19C4, A10B20C4, A10B21C4, A10B22C4, A10B23C4, A10B24C4, A10B25C4, A10B26C4, A10B27C4, A10B28C4, A10B29C4, A10B30C4, A10B31C4, A10B32C4, A10B33C4, A10B34C4, A10B35C4, A10B36C4, A10B37C4, A10B38C4, A10B39C4, A10B40C4, A10B41C4, A10B42C4, A10B43C4, A10B44C4, A10B45C4, A10B46C4, A10B47C4, A10B48C4, A10B49C4, A10B50C4, A10B51C4, A10B52C4, A10B53C4, A10B83C4, A10B1C5, A10B2C5, A10B3C5, A10B4C5, A10B5C5, A10B6C5, A10B7C5, A10B8C5, A10B9C5, A10B10C5, A10B11C5, A10B12C5, A10B13C5, A10B14C5, A10B15C5, A10B16C5, A10B17C5, A10B18C5, A10B19C5, A10B20C5, A10B21C5, A10B22C5, A10B23C5, A10B24C5, A10B25C5, A10B26C5, A10B27C5, A10B28C5, A10B29C5, A10B30C5, A10B31C5, A10B32C5, A10B33C5, A10B34C5, A10B35C5, A10B36C5, A10B37C5, A10B38C5, A10B39C5, A10B40C5, A10B41C5, A10B42C5, A10B43C5, A10B44C5, A10B45C5, A10B46C5, A10B47C5, A10B48C5, A10B49C5, A10B50C5, A10B51C5, A10B52C5, A10B53C5, A10B83C5, A10B1C6, A10B2C6, A10B3C6, A10B4C6, A10B5C6, A10B6C6, A10B7C6, A10B8C6, A10B9C6, A10B10C6, A10B11C6, A10B12C6, A10B13C6, A10B14C6, A10B15C6, A10B16C6, A10B17C6, A10B18C6, A10B19C6, A10B20C6, A10B21C6, A10B22C6, A10B23C6, A10B24C6, A10B25C6, A10B26C6, A10B27C6, A10B28C6, A10B29C6, A10B30C6, A10B31C6, A10B32C6, A10B33C6, A10B34C6, A10B35C6, A10B36C6, A10B37C6, A10B38C6, A10B39C6, A10B40C6, A10B41C6, A10B42C6, A10B43C6, A10B44C6, A10B45C6, A10B46C6, A10B47C6, A10B48C6, A10B49C6, A10B50C6, A10B51C6, A10B52C6, A10B53C6, A10B83C6, A11B1, A11B2, A11B3, A11B4, A11B5, A11B6, A11B7, A11B8, A11B9, A11B10, A11B11, A11B12, A11B13, A11B14, A11B15, A11B16, A11B17, A11B18, A11B19, A11B20, A11B21, A11B22, A11B23, A11B24, A11B25, A11B26, A11B27, A11B28, A11B29, A11B30, A11B31, A11B32, A11B33, A11B34, A11B35, A11B36, A11B37, A11B38, A11B39, A11B40, A11B41, A11B42, A11B43, A11B44, A11B45, A11B46, A11B47, A11B48, A11B49, A11B50, A11B51, A11B52, A11B53, A11B83, A11B54, A11B55, A11B56, A11B57, A11B58, A11B59, A11B60, A11B61, A11B62, A11B63, A11B64, A11B65, A11B66, A11B67, A11B68, A11B69, A11B70, A11B71, A11B72, A11B73, A11B74, A11B75, A11B76, A11B77, A11B78, A11B79, A11B80, A11B81, and A11B82.

In embodiments, the compound is selected from

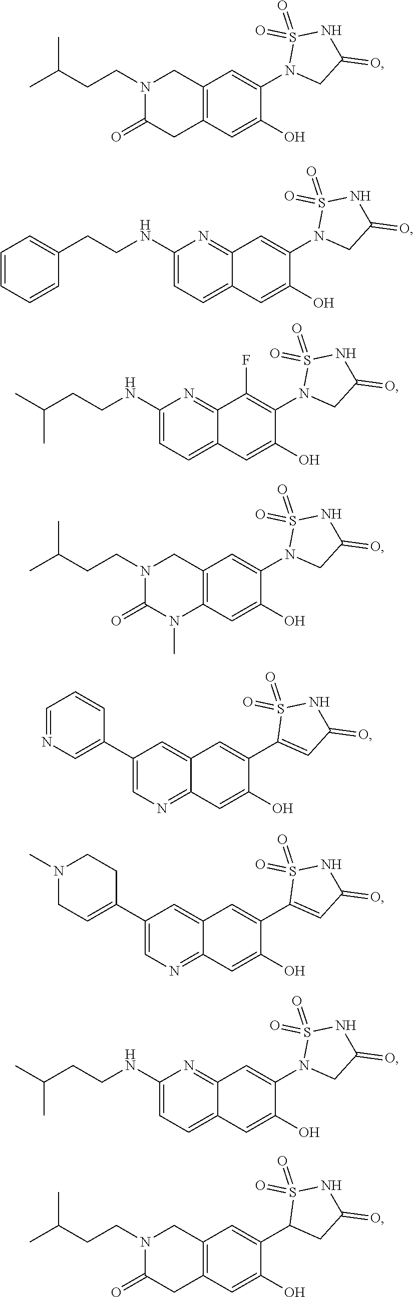

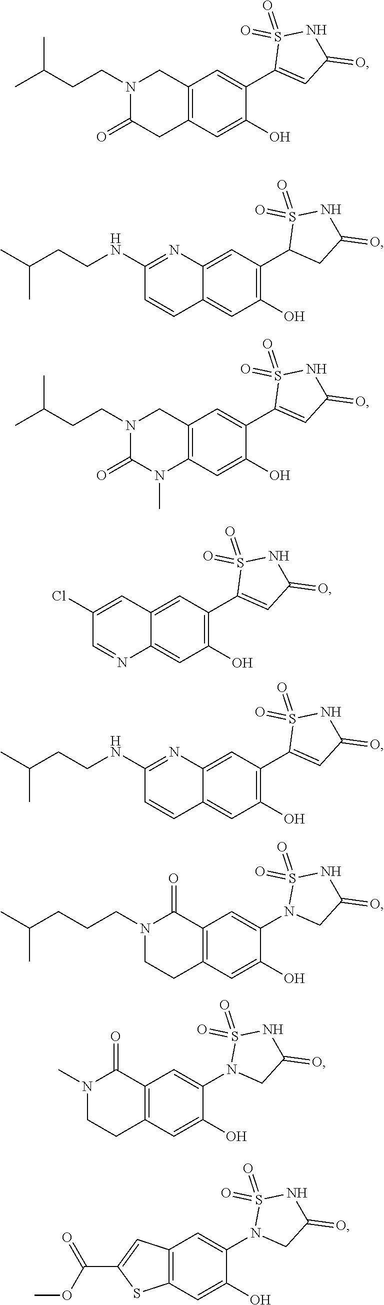

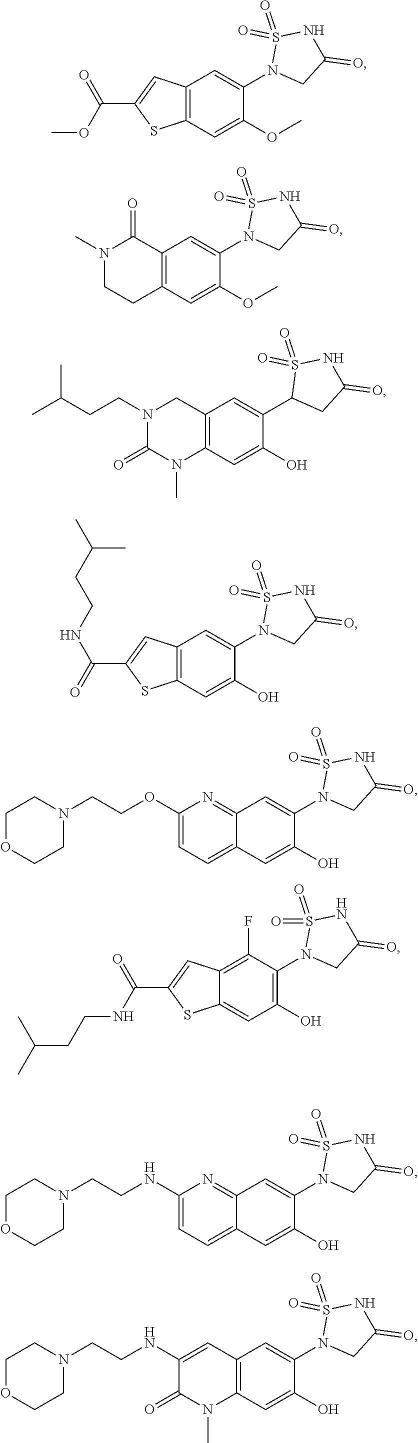

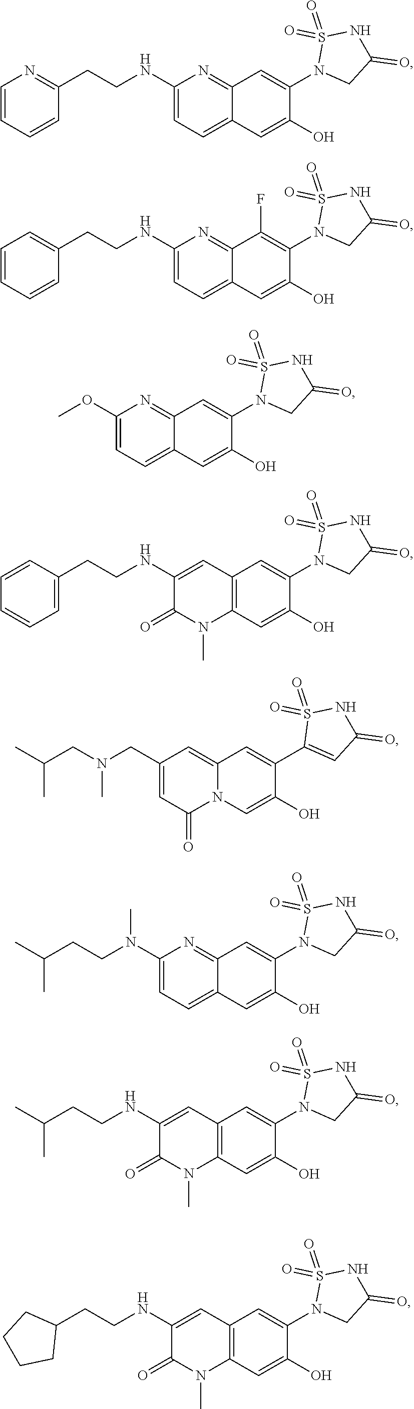

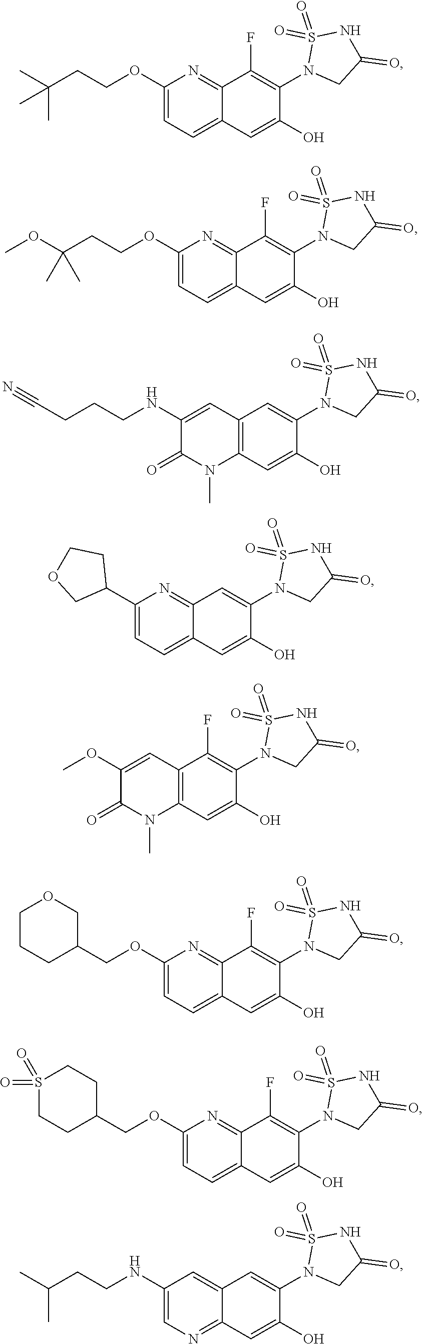

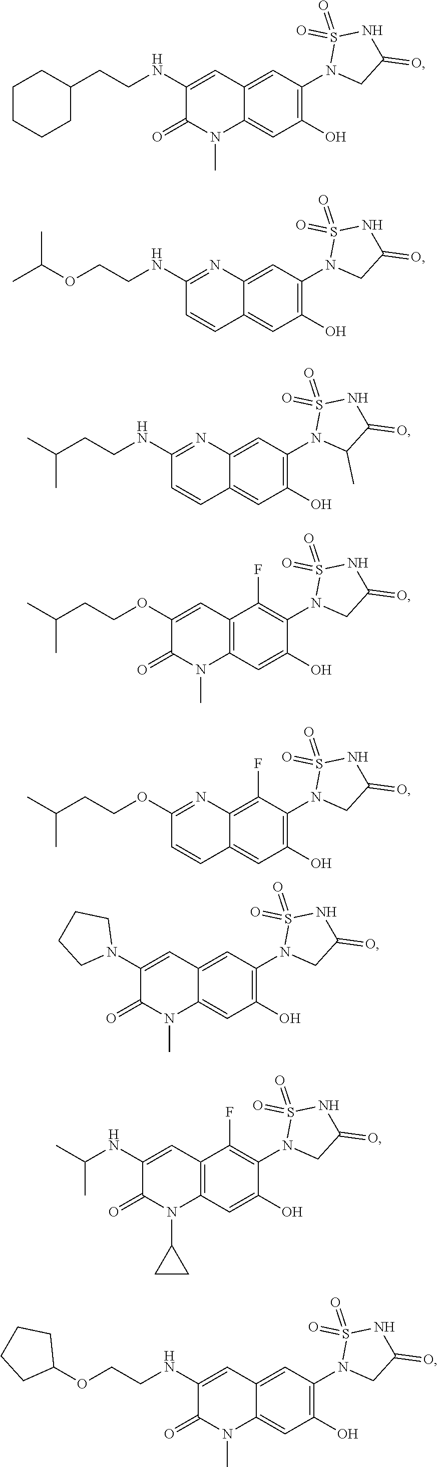

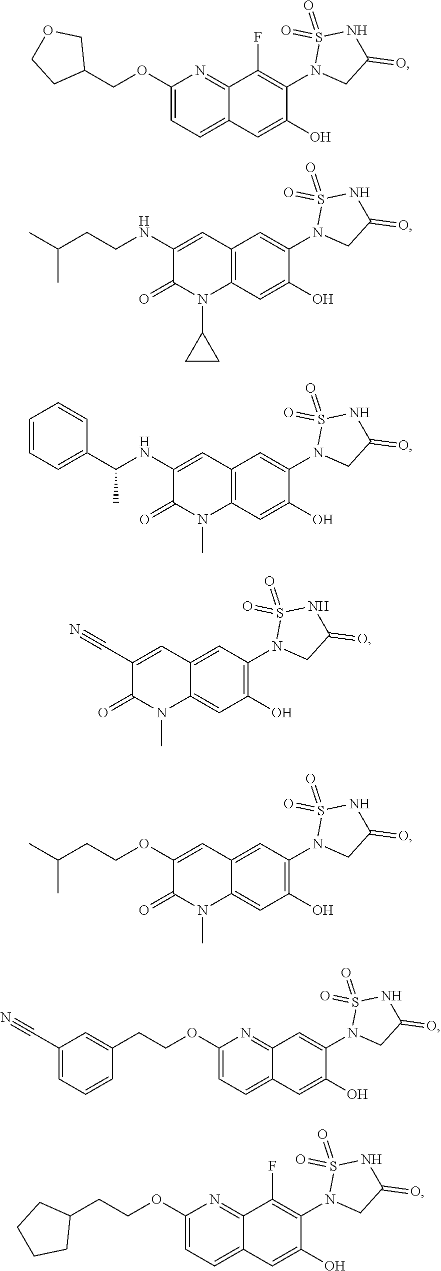

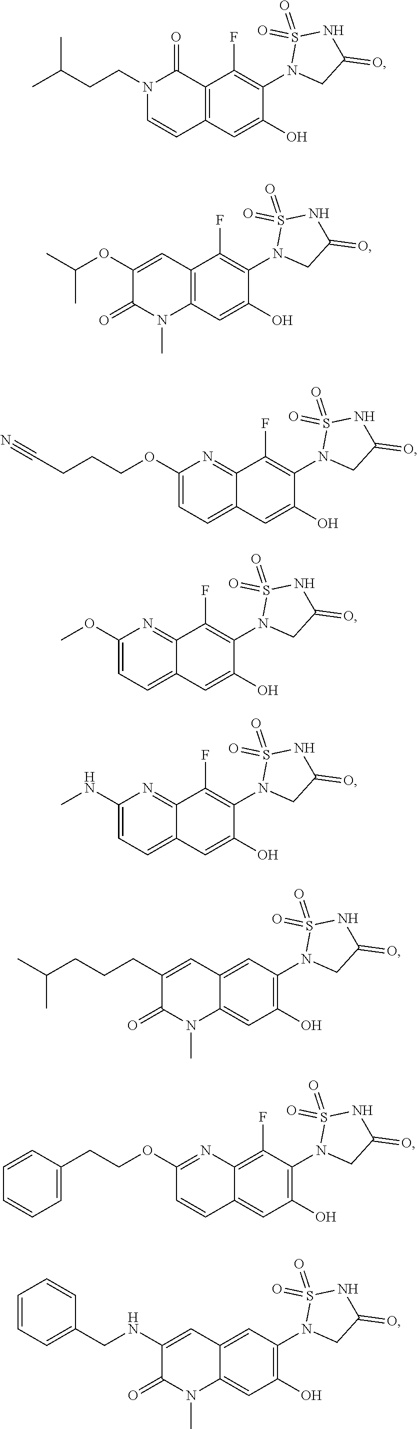

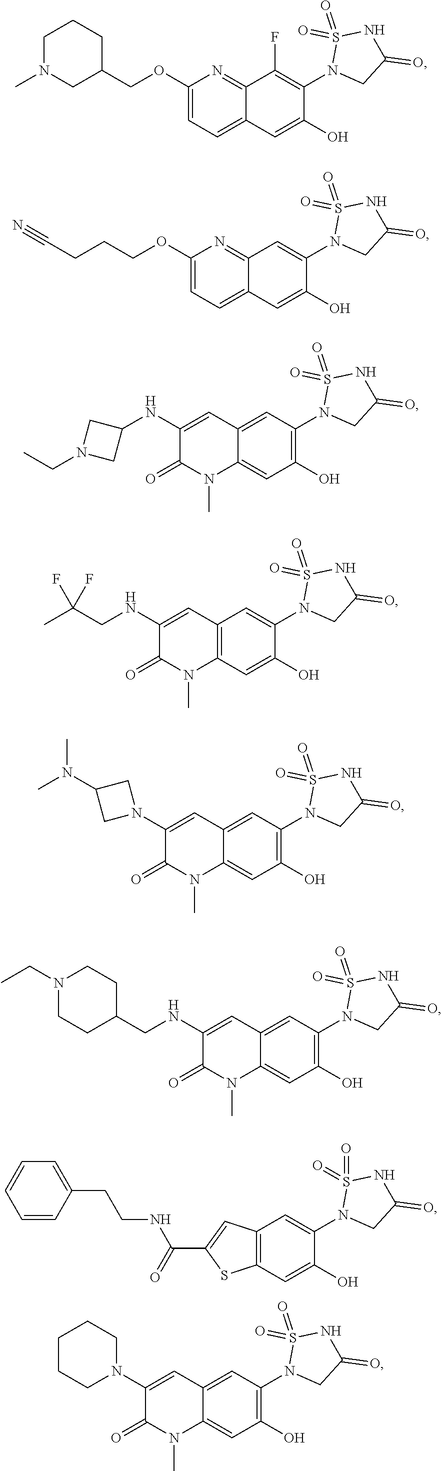

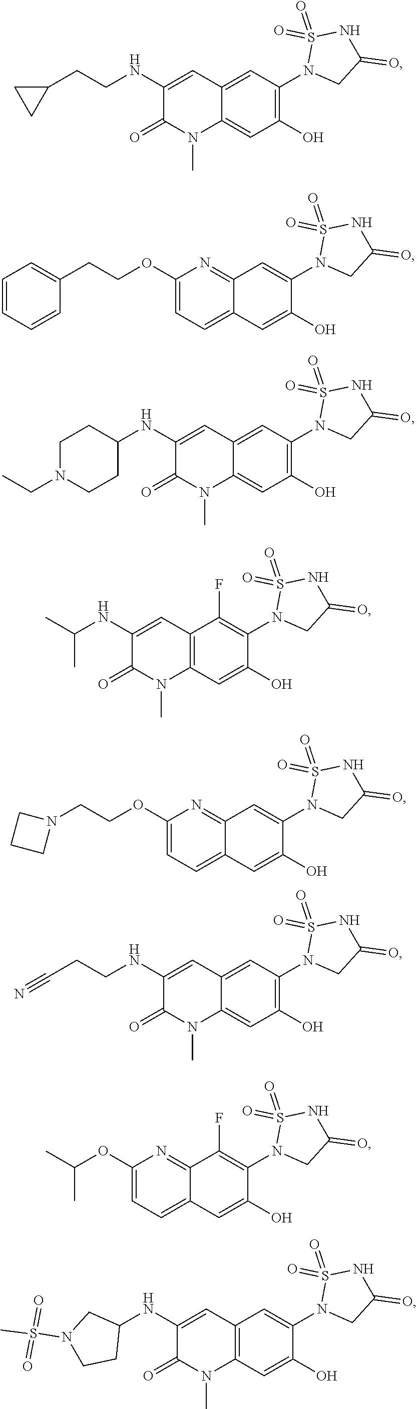

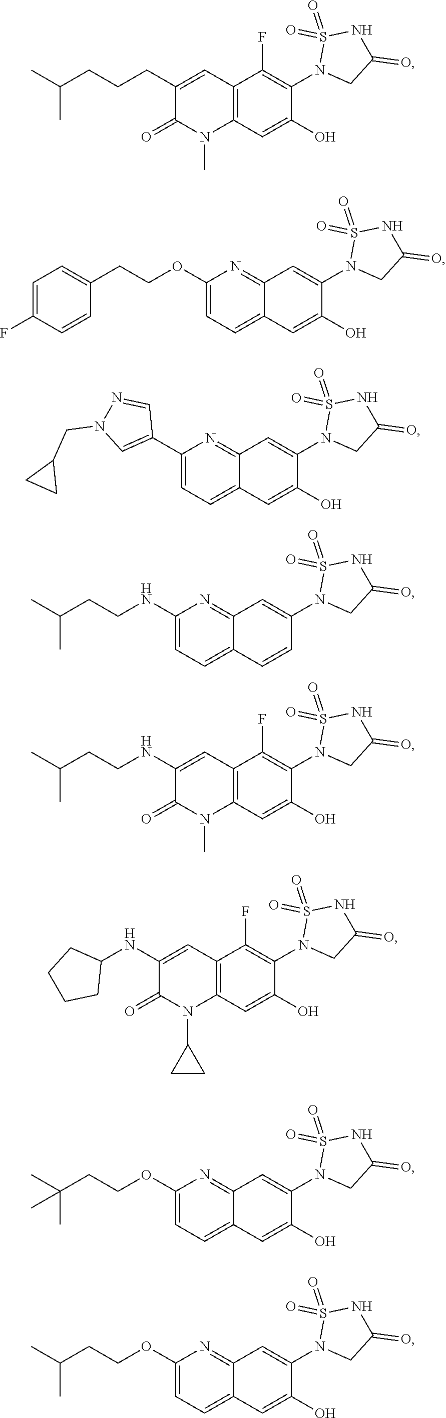

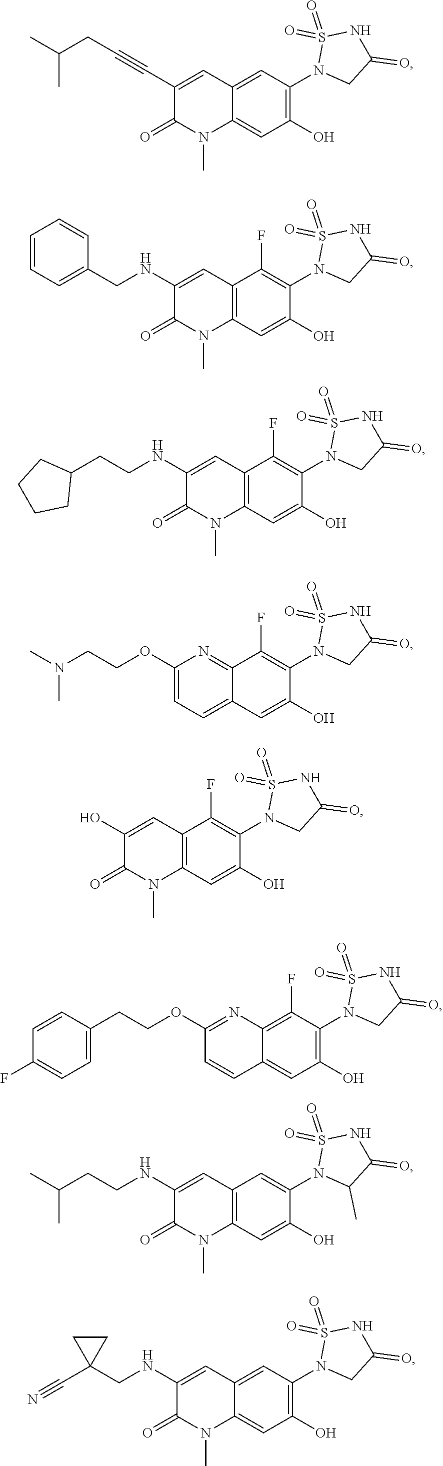

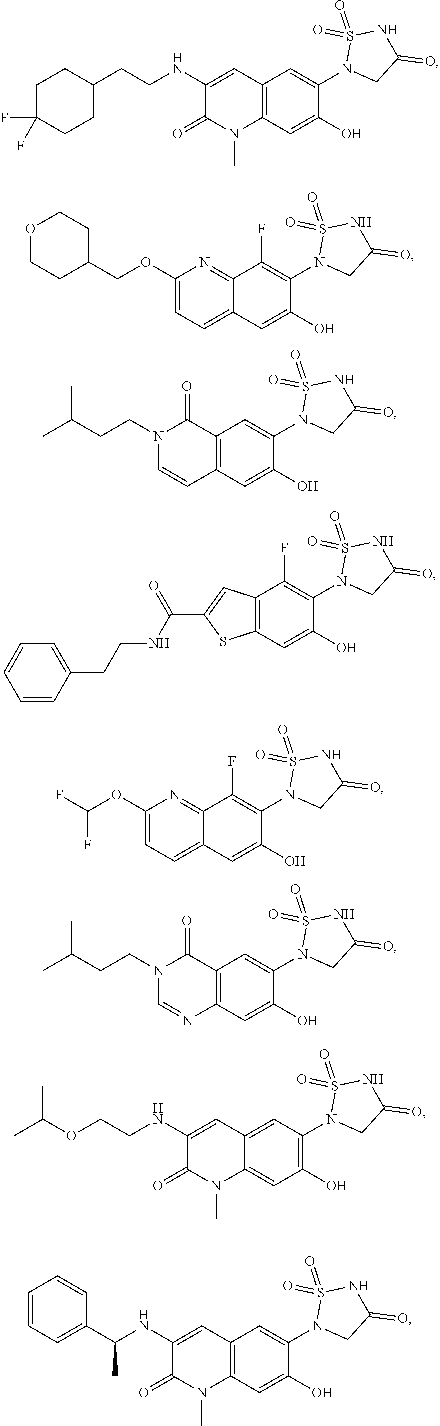

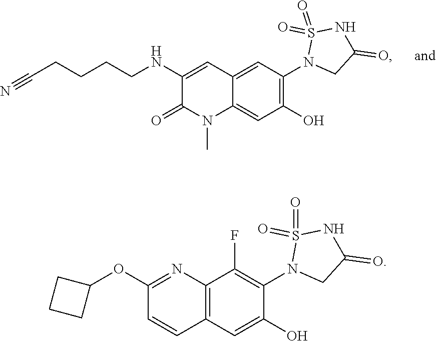 [SMILES: Cn1c(=O)c(NCCCCC#N)cc2cc(N3CC(=O)NS3(=O)=O)c(O)cc21 ; "and" ; O=C1CN(c2c(O)cc3ccc(OC4CCC4)nc3c2F)S(=O)(=O)N1]

In embodiments, the compound is selected from

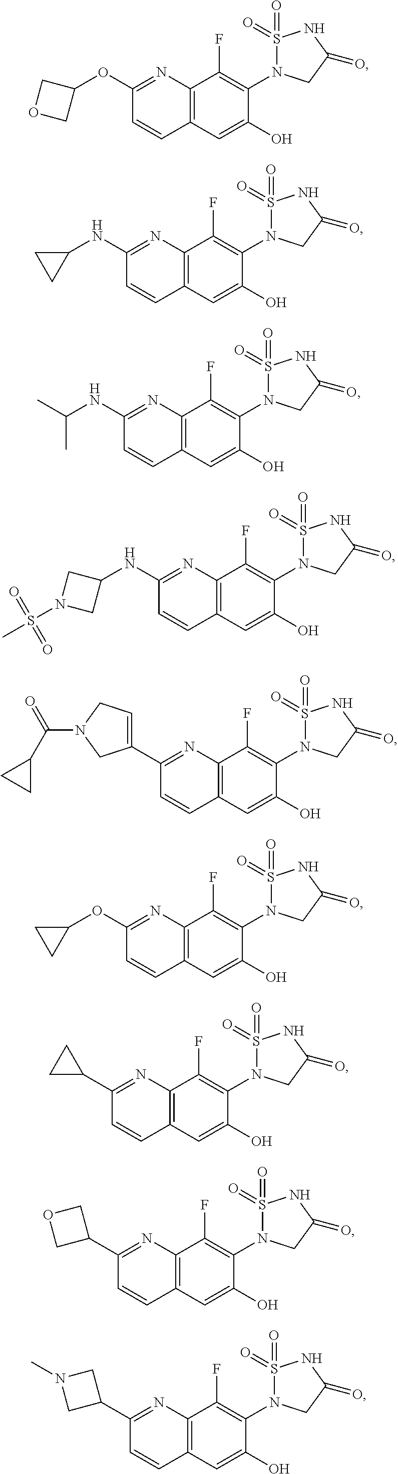

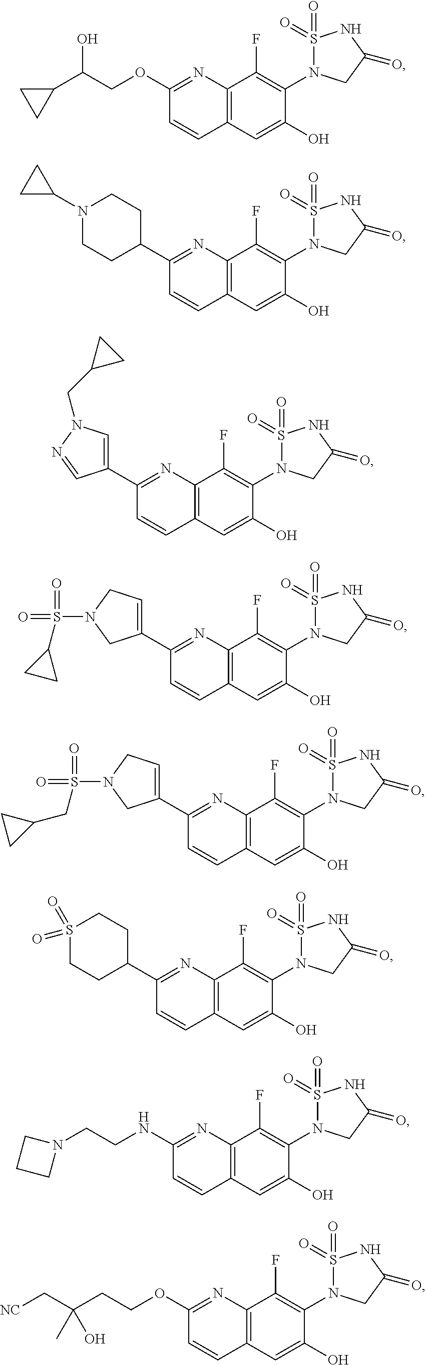

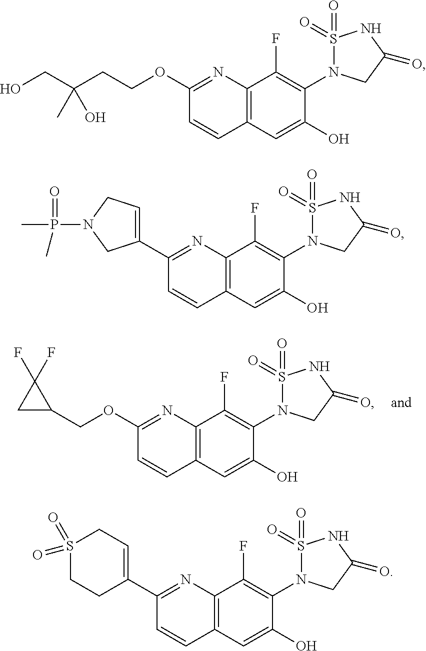

In some embodiments (e.g., including Formulae (I), (II), (III), (IV); or sub-formulae thereof), X is N. In some embodiments (e.g., including Formulae (I), (II), (III), (IV); or sub-formulae thereof), X is C(R⁵). In some embodiments (e.g., including Formulae (I), (II), (III), (IV); or sub-formulae thereof), X is CH. In some embodiments (e.g., including Formulae (I), (II), (III), (IV); or sub-formulae thereof), X is C(halogen). In some embodiments (e.g., including Formulae (I), (II), (III), (IV); or sub-formulae thereof), X is C(F).

In some embodiments (e.g., including Formulae (I), (II), (III), (IV); or sub-formulae thereof), R⁵ is hydrogen. In some embodiments (e.g., including Formulae (I), (II), (III), (IV); or sub-formulae thereof), R⁵ is halogen. In some embodiments (e.g., including Formulae (I), (II), (III), (IV); or sub-formulae thereof), R⁵ is —F. In some embodiments (e.g., including Formulae (I), (II), (III), (IV); or sub-formulae thereof), R⁵ is —Cl. In some embodiments (e.g., including Formulae (I), (II), (III), (IV); or sub-formulae thereof), R⁵ is —Br. In some embodiments (e.g., including Formulae (I), (II), (III), (IV); or sub-formulae thereof), R⁵ is —I. In some embodiments (e.g., including Formulae (I), (II), (III), (IV); or sub-formulae thereof), R⁵ is —CN. In some embodiments (e.g., including Formulae (I), (II), (III), (IV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e}. In some embodiments (e.g., including Formulae (I), (II), (III), (IV); or sub-formulae thereof), R⁵ is C₁₋₆ alkyl. In some embodiments (e.g., including Formulae (I), (II), (III), (IV); or sub-formulae thereof), R⁵ is C₁₋₄alkyl. In some embodiments (e.g., including Formulae (I), (II), (III), (IV); or sub-formulae thereof), R⁵ is C₁₋₃alkyl. In some embodiments (e.g., including Formulae (I), (II), (III), (IV); or sub-formulae thereof), R⁵ is C₁₋₂alkyl. In some embodiments (e.g., including Formulae (I), (II), (III), (IV); or sub-formulae thereof), R⁵ is methyl.

In some embodiments (e.g., including Formulae (I), (II), (III), (IV); or sub-formulae thereof), R⁵ is hydrogen. In some embodiments (e.g., including Formulae (I), (II), (III), (IV); or sub-formulae thereof), R⁵ is halogen. In some embodiments (e.g., including Formulae (I), (II), (III), (IV); or sub-formulae thereof), R⁵ is —CN. In some embodiments (e.g., including Formulae (I), (II), (III), (IV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R⁵ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In some embodiments (e.g., including Formulae (I), (II), (III), (IV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20e}.

In some embodiments (e.g., including Formulae (I), (II), (III), (IV); or sub-formulae thereof), R⁵ is selected from hydrogen, halogen, and C₁₋₆alkyl optionally substituted with one, two, or three R^{20e}. In some embodiments (e.g., including Formulae (I), (II), (III), (IV); or sub-formulae thereof), R⁵ is selected from hydrogen and halogen.

In some embodiments (e.g., including Formulae (I), (II), (III), (IV); or sub-formulae thereof), Y is N. In some embodiments (e.g., including Formulae (I), (II), (III), (IV); or sub-formulae thereof), Y is C(R⁶). In some embodiments (e.g., including Formulae (I), (II), (III), (IV); or sub-formulae thereof), Y is CH. In some embodiments (e.g., including Formulae (I), (II), (III), (IV); or sub-formulae thereof), Y is C(R⁶). In some embodiments (e.g., including Formulae (I), (II), (III), (IV); or sub-formulae thereof), Y is C(OH).

In some embodiments (e.g., including Formulae (I), (II), (III), (IV); or sub-formulae thereof), R⁶ is —OR¹⁶. In some embodiments (e.g., including Formulae (I), (II), (III), (IV); or sub-formulae thereof), R⁶ is —N(R¹²)(R¹⁶). In some embodiments (e.g., including Formulae (I), (II), (III), (IV); or sub-formulae thereof), R⁶ is —OR¹². In some embodiments (e.g., including Formulae (I), (II), (III), (IV); or sub-formulae thereof), R⁶ is —OR¹a. In some embodiments (e.g., including Formulae (I), (II), (III), (IV); or sub-formulae thereof), R⁶ is —SR¹². In some embodiments (e.g., including Formulae (I), (II), (III), (IV); or sub-formulae thereof), R⁶ is —N(R¹²)(R¹³). In some embodiments (e.g., including Formulae (I), (II), (III), (IV); or sub-formulae thereof), R⁶ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl optionally substituted with one, two, or three R^{20f}. In some embodiments (e.g., including Formulae (I), (II), (III), (IV); or sub-formulae thereof), R⁶ is —OH. In some embodiments (e.g., including Formulae (I), (II), (III), (IV); or sub-formulae thereof), R⁶ is C₁₋₆alkyl. In some embodiments (e.g., including Formulae (I), (II), (III), (IV); or sub-formulae thereof), R⁶ is C₁₋₄alkyl. In some embodiments (e.g., including Formulae (I), (II), (III), (IV); or sub-formulae thereof), R⁶ is C₁₋₃alkyl. In some embodiments (e.g., including Formulae (I), (II), (III), (IV); or sub-formulae thereof), R⁶ is C₁₋₂alkyl. In some embodiments (e.g., including Formulae (I), (II), (III), (IV); or sub-formulae thereof), R⁶ is methyl.

In some embodiments (e.g., including Formulae (I), (II), (III), (IV); or sub-formulae thereof), R⁶ is hydrogen. In some embodiments (e.g., including Formulae (I), (II), (III), (IV); or sub-formulae thereof), R⁶ is halogen. In some embodiments (e.g., including Formulae (I), (II), (III), (IV); or sub-formulae thereof), R⁶ is —CN. In some embodiments (e.g., including Formulae (I), (II), (III), (IV); or sub-formulae thereof), R⁶ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R⁶ is —OR¹⁶. In some embodiments (e.g., including Formulae (I), (II), (III), (IV); or sub-formulae thereof), R⁶ is —N(R¹²)(R¹⁶). In some embodiments (e.g., including Formulae (I), (II), (III), (IV); or sub-formulae thereof), R⁶ is —OR¹². In some embodiments (e.g., including Formulae (I), (II), (III), (IV); or sub-formulae thereof), R⁶ is —OR¹². In some embodiments (e.g., including Formulae (I), (II), (III), (IV); or sub-formulae thereof), R⁶ is —SR¹². In some embodiments (e.g., including Formulae (I), (II), (III), (IV); or sub-formulae thereof), R⁶ is —N(R¹²)(R¹³). In some embodiments (e.g., including Formulae (I), (II), (III), (IV); or sub-formulae thereof), R⁶ is —C(O)OR¹². In some embodiments (e.g., including Formulae (I), (II), (III), (IV); or sub-formulae thereof), R⁶ is —OC(O)N(R¹²)(R¹³). In some embodiments (e.g., including Formulae (I), (II), (III), (IV); or sub-formulae thereof), R⁶ is —N(R¹⁴)C(O)N(R¹²)(R¹³). In some embodiments (e.g., including Formulae (I), (II), (III), (IV); or sub-formulae thereof), R⁶ is —N(R¹⁴)C(O)OR¹². In some embodiments (e.g., including Formulae (I), (II), (III), (IV); or sub-formulae thereof), R⁶ is —N(R¹⁴)S(O)₂R¹⁵. In some embodiments (e.g., including Formulae (I), (II), (III), (IV); or sub-formulae thereof), R⁶ is —C(O)R¹⁵. In some embodiments (e.g., including Formulae (I), (II), (III), (IV); or sub-formulae thereof), R⁶ is —S(O)R¹⁵. In some embodiments (e.g., including Formulae (I), (II), (III), (IV); or sub-formulae thereof), R⁶ is —OC(O)R¹⁵. In some embodiments (e.g., including Formulae (I), (II), (III), (IV); or sub-formulae thereof), R⁶ is —C(O)N(R¹²)(R¹³). In some embodiments (e.g., including Formulae (I), (II), (III), (IV); or sub-formulae thereof), R⁶ is —C(O)C(O)N(R¹²)(R¹³). In some embodiments (e.g., including Formulae (I), (II), (III), (IV); or sub-formulae thereof), R⁶ is —N(R¹⁴)C(O)R¹⁵. In some embodiments (e.g., including Formulae (I), (II), (III), (IV); or sub-formulae thereof), R⁶ is —S(O)₂R¹⁵. In some embodiments (e.g., including Formulae (I), (II), (III), (IV); or sub-formulae thereof), R⁶ is —S(O)₂N(R¹²)(R¹³). In some embodiments (e.g., including Formulae (I), (II), (III), (IV); or sub-formulae thereof), R⁶ is —S(═O)(═NH)N(R¹²)(R¹³). In some embodiments (e.g., including Formulae (I), (II), (III), (IV); or sub-formulae thereof), R⁶ is —CH₂C(O)N(R¹²)(R¹³). In some embodiments (e.g., including Formulae (I), (II), (III), (IV); or sub-formulae thereof), R⁶ is —CH₂N(R¹⁴)C(O)R¹⁵. In some embodiments (e.g., including Formulae (I), (II), (III), (IV); or sub-formulae thereof), R⁶ is —CH₂S(O)₂R¹⁵. In some embodiments (e.g., including Formulae (I), (II), (III), (IV); or sub-formulae thereof), R⁶ is —CH₂S(O)₂N(R¹²)(R¹³). In some embodiments (e.g., including Formulae (I), (II), (III), (IV); or sub-formulae thereof), R⁶ is C₁₋₆ alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20f}. In some embodiments (e.g., including Formulae (I), (II), (III), (IV); or sub-formulae thereof), R⁶ is —OH.

In some embodiments (e.g., including Formulae (I), (II), (III), (IV); or sub-formulae thereof), R⁶ is selected from hydrogen, halogen, —OR^{12a}, and C₁₋₆alkyl optionally substituted with one, two, or three R^{20f}.

In some embodiments of Formulae (I), or sub-formulae thereof, J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of Formulae (I), or sub-formulae thereof, J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of Formulae (I), or sub-formulae thereof, J³ is CH₂ and R⁶ is —OR¹⁶. In some embodiments of Formulae (I), or sub-formulae thereof, J³ is CH₂ and R⁶ is —N(R¹²)(R¹⁶).

In some embodiments (e.g., including Formulae (I), (II), (III), (IV); or sub-formulae thereof), Z is N. In some embodiments (e.g., including Formulae (I), (II), (III), (IV); or sub-formulae thereof), Z is C(R⁷). In some embodiments (e.g., including Formulae (I), (II), (III), (IV); or sub-formulae thereof), Z is CH. In some embodiments (e.g., including Formulae (I), (II), (III), (IV); or sub-formulae thereof), Z is C(R⁷).

In some embodiments (e.g., including Formulae (I), (II), (III), (IV); or sub-formulae thereof), R⁷ is hydrogen. In some embodiments (e.g., including Formulae (I), (II), (III), (IV); or sub-formulae thereof), R⁷ is halogen. In some embodiments (e.g., including Formulae (I), (II), (III), (IV); or sub-formulae thereof), R⁷ is —CN. In some embodiments (e.g., including Formulae (I), (II), (III), (IV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g}. In some embodiments (e.g., including Formulae (I), (II), (III), (IV); or sub-formulae thereof), R⁷ is C₁₋₆alkyl. In some embodiments (e.g., including Formulae (I), (II), (III), (IV); or sub-formulae thereof), R⁷ is C₁₋₄alkyl. In some embodiments (e.g., including Formulae (I), (II), (III), (IV); or sub-formulae thereof), R⁷ is C₁₋₂alkyl. In some embodiments (e.g., including Formulae (I), (II), (III), (IV); or sub-formulae thereof), R⁷ is methyl.

In some embodiments (e.g., including Formulae (I), (II), (III), (IV); or sub-formulae thereof), R⁷ is hydrogen. In some embodiments (e.g., including Formulae (I), (II), (III), (IV); or sub-formulae thereof), R⁷ is halogen. In some embodiments (e.g., including Formulae (I), (II), (III), (IV); or sub-formulae thereof), R⁷ is —CN. In some embodiments (e.g., including Formulae (I), (II), (III), (IV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20g}. In some embodiments (e.g., including Formulae (I), (II), (III), (IV); or sub-formulae thereof), R⁷ is selected from hydrogen, halogen, and C₁₋₆alkyl optionally substituted with one, two, or three R^{20g}.

In some embodiments (e.g., including Formulae (I), (II), (III), (IV); or sub-formulae thereof), R⁸ is hydrogen. In some embodiments (e.g., including Formulae (I), (II), (III), (IV); or sub-formulae thereof), R⁸ is halogen. In some embodiments (e.g., including Formulae (I), (II), (III), (IV); or sub-formulae thereof), R⁸ is —CN. In some embodiments (e.g., including Formulae (I), (II), (III), (IV); or sub-formulae thereof), R⁸ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R⁸ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20h}.

In some embodiments (e.g., including Formulae (I), (II), (III), (IV); or sub-formulae thereof), R⁹ is hydrogen. In some embodiments (e.g., including Formulae (I), (II), (III), (IV); or sub-formulae thereof), R⁹ is halogen. In some embodiments (e.g., including Formulae (I), (II), (III), (IV); or sub-formulae thereof), R⁹ is —CN. In some embodiments (e.g., including Formulae (I), (II), (III), (IV); or sub-formulae thereof), R⁹ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R⁹ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R⁹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R²⁰ⁱ.

In some embodiments (e.g., including Formulae (I), (II), (III), (IV); or sub-formulae thereof), R^{9a} is hydrogen. In some embodiments (e.g., including Formulae (I), (II), (III), (IV); or sub-formulae thereof), R^{9a} is halogen. In some embodiments (e.g., including Formulae (I), (II), (III), (IV); or sub-formulae thereof), R^{9a} is —CN. In some embodiments (e.g., including Formulae (I), (II), (III), (IV); or sub-formulae thereof), R^{9a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R^{9a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R^{9a} is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R²⁰ⁱ.

In some embodiments (e.g., including Formulae (I), (II), (III), (IV); or sub-formulae thereof), R⁹ and R^{9a} are combined to form a C₃₋₆cycloalkyl. In some embodiments (e.g., including Formulae (I), (II), (III), (IV); or sub-formulae thereof), R⁹ and R^{9a} are combined to form a C₂₋₉heterocycloalkyl. In some embodiments (e.g., including Formulae (I), (II), (III), (IV); or sub-formulae thereof), R⁹ and R^{9a} are combined to form a C₃₋₆cycloalkyl substituted with one, two, or three R²⁰ⁱ. In some embodiments (e.g., including Formulae (I), (II), (III), (IV); or sub-formulae thereof), R⁹ and R^{9a} are combined to form a C₂₋₉heterocycloalkyl substituted with one, two, or three R²⁰ⁱ.

In some embodiments (e.g., including Formulae (I), (II), (III), (IV); or sub-formulae thereof), R⁹ and R^{9a} are hydrogen.

In some embodiments (e.g., including Formulae (I), (II), (III), (IV); or sub-formulae thereof), R¹⁰ is hydrogen. In some embodiments (e.g., including Formulae (I), (II), (III), (IV); or sub-formulae thereof), R¹⁰ is halogen. In some embodiments (e.g., including Formulae (I), (II), (III), (IV); or sub-formulae thereof), R¹⁰ is —CN. In some embodiments (e.g., including Formulae (I), (II), (III), (IV); or sub-formulae thereof), R¹⁰ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R¹⁰ is —R¹⁶. In some embodiments (e.g., including Formulae (I), (II), (III), (IV); or sub-formulae thereof), R¹⁰ is —OR¹⁶. In some embodiments (e.g., including Formulae (I), (II), (III), (IV); or sub-formulae thereof), R¹⁰ is —N(R¹²)(R¹⁶). In some embodiments (e.g., including Formulae (I), (II), (III), (IV); or sub-formulae thereof), R¹⁰ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R¹⁰ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20j}.

In some embodiments (e.g., including Formulae (I), (II), (III), (IV); or sub-formulae thereof), R^{10a} is hydrogen. In some embodiments (e.g., including Formulae (I), (II), (III), (IV); or sub-formulae thereof), R^{10a} is halogen. In some embodiments (e.g., including Formulae (I), (II), (III), (IV); or sub-formulae thereof), R^{10a} is —CN. In some embodiments (e.g., including Formulae (I), (II), (III), (IV); or sub-formulae thereof), R^{10a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R^{10a} is —R¹⁶. In some embodiments (e.g., including Formulae (I), (II), (III), (IV); or sub-formulae thereof), R^{10a} is —OR¹⁶. In some embodiments (e.g., including Formulae (I), (II), (III), (IV); or sub-formulae thereof), R^{10a} is —N(R¹²)(R¹⁶). In some embodiments (e.g., including Formulae (I), (II), (III), (IV); or sub-formulae thereof), R^{10a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R^{10a} is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20j}.

In some embodiments (e.g., including Formulae (I), (II), (III), (IV); or sub-formulae thereof), R¹⁰ and R^{10a} are combined to form a C₃₋₆cycloalkyl. In some embodiments (e.g., including Formulae (I), (II), (III), (IV); or sub-formulae thereof), R¹⁰ and R^{10a} are combined to form a C₂₋₉heterocycloalkyl. In some embodiments (e.g., including Formulae (I), (II), (III), (IV); or sub-formulae thereof), R¹⁰ and R^{10a} are combined to form a C₃₋₆cycloalkyl substituted with one, two, or three R^{20j}. In some embodiments (e.g., including Formulae (I), (II), (III), (IV); or sub-formulae thereof), R¹⁰ and R^{10a} are combined to form a C₂₋₉heterocycloalkyl substituted with one, two, or three R^{20j}.

In some embodiments (e.g., including Formulae (I), (II), (III), (IV); or sub-formulae thereof), J¹ is N. In some embodiments (e.g., including Formulae (I), (II), (III), (IV); or sub-formulae thereof), J¹ is C. In some embodiments (e.g., including Formulae (I), (II), (III), (IV); or sub-formulae thereof), J¹ is C(R⁸).

In some embodiments (e.g., including Formulae (I), (II), (III), (IV); or sub-formulae thereof), J² is N. In some embodiments (e.g., including Formulae (I), (II), (III), (IV); or sub-formulae thereof), J² is N(R⁹). In some embodiments (e.g., including Formulae (I), (II), (III), (IV); or sub-formulae thereof), J² is C(R⁹). In some embodiments (e.g., including Formulae (I), (II), (III), (IV); or sub-formulae thereof), J² is C(H). In some embodiments (e.g., including Formulae (I), (II), (III), (IV); or sub-formulae thereof), J² is C(R⁹)(R^{9a}). In some embodiments (e.g., including Formulae (I), (II), (III), (IV); or sub-formulae thereof), J² is CH₂. In some embodiments (e.g., including Formulae (I), (II), (III), (IV); or sub-formulae thereof), J² is C(O).

In some embodiments (e.g., including Formulae (I), (II), (III), (IV); or sub-formulae thereof), J³ is N(R¹⁰). In some embodiments (e.g., including Formulae (I), (II), (III), (IV); or sub-formulae thereof), J³ is C(R¹⁰)(R^{10a}). In some embodiments (e.g., including Formulae (I), (II), (III), (IV); or sub-formulae thereof), J³ is NH.

In some embodiments of (e.g., of Formulae (I), or sub-formulae thereof), J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶) OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of (e.g., of Formulae (I), or sub-formulae thereof), J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶) SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_9 S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of (e.g., of Formulae (I)), or sub-formulae thereof, J³ is CH₂ and R⁶ is —OR¹⁶. In some embodiments of (e.g., of Formulae (I)), or sub-formulae thereof, J³ is CH₂ and R⁶ is —N(R¹²)(R¹⁶).

In some embodiments (e.g., including Formulae (I), (II), (III), (IV); or sub-formulae thereof), J¹ is N and J² is C(R⁹)(R^{9a}). In some embodiments (e.g., including Formulae (I), (II), (III), (IV); or sub-formulae thereof), J¹ is C(R⁸) and J² is C(R⁹)(R^{9a}). In some embodiments (e.g., including Formulae (I), (II), (III), (IV); or sub-formulae thereof), J¹ is C and J² is C(R⁹). In some embodiments (e.g., including Formulae (I), (II), (III), (IV); or sub-formulae thereof), J¹ is N and J² is C(R⁹)(R^{9a}). In some embodiments (e.g., including Formulae (I), (II), (III), (IV); or sub-formulae thereof), J¹ is C(R¹) and J² is C(R⁹)(R^{9a}). In some embodiments (e.g., including Formulae (I), (II), (III), (IV); or sub-formulae thereof), J¹ is C and J² is C(R⁹).

In some embodiments, R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, when J² is N(R⁹); R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, when J² is N(R⁹); R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

In some embodiments, R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, when J³ is N(R¹⁰); R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, when J³ is N(R¹⁰); R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}.

In some embodiments (e.g., including Formulae (I), (II), (III), (IV); or sub-formulae thereof), W¹ is N. In some embodiments (e.g., including Formulae (I), (II), (III), (LV); or sub-formulae thereof), W¹ is N(R¹). In some embodiments (e.g., including Formulae (I), (II), (III), (IV); or sub-formulae thereof), W¹ is O. In some embodiments (e.g., including Formulae (I), (II), (III), (IV); or sub-formulae thereof), W¹ is C(R¹). In some embodiments (e.g., including Formulae (I), (II), (III), (IV); or sub-formulae thereof), W¹ is C(R¹)(R^{1a}). In some embodiments (e.g., including Formulae (I), (II), (III), (IV); or sub-formulae thereof), W¹ is C(O). In some embodiments (e.g., including Formulae (I), (II), (III), (IV); or sub-formulae thereof), W¹ is S(O). In some embodiments (e.g., including Formulae (I), (II), (III), (IV); or sub-formulae thereof), W¹ is S(O)₂. In some embodiments (e.g., including Formulae (I), (II), (III), (IV); or sub-formulae thereof), W¹ is S(O)(NR¹). In some embodiments (e.g., including Formulae (I), (II), (III), (IV); or sub-formulae thereof), W¹ is S(O)(R¹). In some embodiments (e.g., including Formulae (I), (II), (III), (IV); or sub-formulae thereof), W¹ is P(O)(R¹).

In some embodiments (e.g., including Formulae (I), (II), (III), (IV); or sub-formulae thereof), R¹ is hydrogen. In some embodiments (e.g., including Formulae (I), (II), (III), (IV); or sub-formulae thereof), R¹ is halogen. In some embodiments (e.g., including Formulae (I), (II), (III), (IV); or sub-formulae thereof), R¹ is —CN. In some embodiments (e.g., including Formulae (I), (II), (III), (IV); or sub-formulae thereof), R¹ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (IV); or sub-formulae thereof), R¹ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R¹ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20a}.

In some embodiments, R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, when W¹ is N(R¹), S(O)(NR¹), S(O)(R¹), or P(O)(R¹); R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, when W¹ is N(R¹), S(O)(NR¹), S(O)(R¹), or P(O)(R¹); R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, R¹ is —CN. In some embodiments, R¹ is —F. In some embodiments, R¹ is —CF₃. In some embodiments, R¹ is —OCH₃. In some embodiments, R¹ is cyclopropyl. In some embodiments, R¹ is —OCH₂CF₃. In some embodiments, R¹ is —CH₃.

In some embodiments (e.g., including Formulae (I), (II), (III), (IV); or sub-formulae thereof), R^{1a} is hydrogen. In some embodiments (e.g., including Formulae (I), (II), (III), (IV); or sub-formulae thereof), R^{1a} is halogen. In some embodiments (e.g., including Formulae (I), (II), (III), (IV); or sub-formulae thereof), R^{1a} is —CN. In some embodiments (e.g., including Formulae (I), (II), (III), (IV); or sub-formulae thereof), R^{1a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl. In some embodiments (e.g., including Formulae (I), (II), (III), (LV); or sub-formulae thereof), R^{1a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R^{1a} is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20a}.

In some embodiments (e.g., including Formulae (I), (II), (III), (IV); or sub-formulae thereof), R¹ and R^{1a} are combined to form a C₃₋₆cycloalkyl. In some embodiments (e.g., including Formulae (I), (II), (III), (IV); or sub-formulae thereof), R¹ and R^{1a} are combined to form a C₂₋₉heterocycloalkyl. In some embodiments (e.g., including Formulae (I), (II), (III), (IV); or sub-formulae thereof), R¹ and R^{1a} are combined to form a C₃₋₆cycloalkyl substituted with one, two, or three R^{20a}. In some embodiments (e.g., including Formulae (I), (II), (III), (IV); or sub-formulae thereof), R¹ and R^{1a} are combined to form a C₂₋₉heterocycloalkyl substituted with one, two, or three R^{20a}.

In some embodiments (e.g., including Formulae (I), (II), (III), (IV); or sub-formulae thereof), W² is N. In some embodiments (e.g., including Formulae (I), (II), (III), (IV); or sub-formulae thereof), W² is N(R²). In some embodiments (e.g., including Formulae (I), (II), (III), (IV); or sub-formulae thereof), W² is O. In some embodiments (e.g., including Formulae (I), (II), (III), (IV); or sub-formulae thereof), W² is C(R²). In some embodiments (e.g., including Formulae (I), (II), (III), (IV); or sub-formulae thereof), W² is C(R²)(R^{2a}). In some embodiments (e.g., including Formulae (I), (II), (III), (IV); or sub-formulae thereof), W² is C(O). In some embodiments (e.g., including Formulae (I), (II), (III), (IV); or sub-formulae thereof), W² is S(O). In some embodiments (e.g., including Formulae (I), (II), (III), (IV); or sub-formulae thereof), W² is S(O)₂. In some embodiments (e.g., including Formulae (I), (II), (III), (IV); or sub-formulae thereof), W² is S(O)(NR²). In some embodiments (e.g., including Formulae (I), (II), (III), (IV); or sub-formulae thereof), W² is S(O)(R²). In some embodiments (e.g., including Formulae (I), (II), (III), (IV); or sub-formulae thereof), W² is P(O)(R²).

In some embodiments, R² is independently hydrogen. In some embodiments, R² is independently halogen. In some embodiments, R² is independently —CN. In some embodiments, R² is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments, R² is independently —OR¹². In some embodiments, R² is independently —SR¹². In some embodiments, R² is independently —N(R¹²)(R¹³). In some embodiments, R² is independently —C(O)OR¹². In some embodiments, R² is independently —OC(O)N(R¹²)(R¹³). In some embodiments, R² is independently —N(R¹⁴)C(O)N(R¹²)(R¹³). In some embodiments, R² is independently —N(R¹⁴)C(O)OR¹². In some embodiments, R² is independently —N(R¹⁴)S(O)₂R¹⁵. In some embodiments, R² is independently —C(O)R¹⁵. In some embodiments, R² is independently —S(O)R¹⁵. In some embodiments, R² is independently —OC(O)R¹⁵. In some embodiments, R² is independently —C(O)N(R¹²)(R¹³). In some embodiments, R² is independently —C(O)C(O)N(R¹²)(R¹³). In some embodiments, R² is independently —N(R¹⁴)C(O)R¹⁵. In some embodiments, R² is independently —S(O)₂R¹⁵. In some embodiments, R² is independently —S(O)₂N(R¹²)(R¹³). In some embodiments, R² is independently —S(═O)(═NH)N(R¹²)(R¹³). In some embodiments, R² is independently —CH₂C(O)N(R¹²)(R¹³). In some embodiments, R² is independently —CH₂N(R¹⁴)C(O)R¹⁵. In some embodiments, R² is independently —CH₂S(O)₂R¹⁵. In some embodiments, R² is independently —CH₂S(O)₂N(R¹²)(R¹³). In some embodiments, R² is independently C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20b}. In some embodiments, R² is independently C₁₋₆alkyl substituted with one, two, or three R^{20b}. In some embodiments, R² is independently C₂₋₆alkenyl substituted with one, two, or three R^{20b}. In some embodiments, R² is independently C₂₋₆alkynyl substituted with one, two, or three R^{20b}. In some embodiments, R² is independently C₃₋₆cycloalkyl substituted with one, two, or three R^{20b}. In some embodiments, R² is independently C₂₋₉heterocycloalkyl substituted with one, two, or three R²¹. In some embodiments, R² is independently C₆₋₁₀aryl substituted with one, two, or three R^{20b}. In some embodiments, R² is independently C₁₋₉heteroaryl substituted with one, two, or three R^{20b}. In some embodiments, R² is independently C₁₋₆alkyl. In some embodiments, R² is independently C₂₋₆alkenyl. In some embodiments, R² is independently C₂₋₆alkynyl. In some embodiments, R² is independently C₃₋₆cycloalkyl. In some embodiments, R² is independently C₂₋₉heterocycloalkyl. In some embodiments, R² is independently C₆₋₁₀aryl. In some embodiments, R² is independently C₁₋₉heteroaryl. In some embodiments, R² is independently C₁alkyl. In some embodiments, R² is independently C₂alkyl. In some embodiments, R² is independently C₃alkyl. In some embodiments, R² is independently C₄alkyl. In some embodiments, R² is independently C₅alkyl. In some embodiments, R² is independently C₆alkyl.

In some embodiments, R² is independently —OH. In some embodiments, R² is independently —SH. In some embodiments, R² is independently —NH₂. In some embodiments, R² is independently —C(O)OH. In some embodiments, R² is independently —OC(O)NH₂. In some embodiments, R² is independently —NHC(O)NH₂. In some embodiments, R² is independently —NHC(O)OH. In some embodiments, R² is independently —NHS(O)₂R¹⁵. In some embodiments, R² is independently —C(O)R¹⁵. In some embodiments, R² is independently —S(O)R¹⁵. In some embodiments, R² is independently —OC(O)R¹⁵. In some embodiments, R² is independently —OC(O)CH₃. In some embodiments, R² is independently —C(O)NH₂. In some embodiments, R² is independently —C(O)C(O)NH₂. In some embodiments, R² is independently —NHC(O)R¹⁵. In some embodiments, R² is independently —S(O)₂R¹⁵. In some embodiments, R² is independently —S(O)₂N(R¹²)(R¹³). In some embodiments, R² is independently —S(═O)(═NH)NH₂. In some embodiments, R² is independently —CH₂C(O)NH₂. In some embodiments, R² is independently —CH₂NHC(O)R¹⁵. In some embodiments, R² is independently —CH₂S(O)₂R¹⁵. In some embodiments, R² is independently —CH₂S(O)₂NH₂.

In some embodiments, R² is independently —NH(R¹²). In some embodiments, R² is independently —OC(O)N(R¹²)(R¹³). In some embodiments, R² is independently —NHC(O)N(R¹²). In some embodiments, R² is independently —NHC(O)OR¹². In some embodiments, R² is independently —C(O)N(R¹²)(R¹³). In some embodiments, R² is independently —C(O)C(O)NH(R¹²). In some embodiments, R² is independently —S(O)₂NH(R¹²). In some embodiments, R² is independently —S(═O)(═NH)NH(R¹²). In some embodiments, R² is independently —CH₂C(O)NH(R¹²). In some embodiments, R² is independently —CH₂S(O)₂NH(R¹²).

In some embodiments, R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In some embodiments, R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In some embodiments, when W² is N(R²), S(O)(NR²), S(O)(R²), or P(O)(R²) R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²¹. In some embodiments, when W² is N(R²), S(O)(NR²), S(O)(R²), or P(O)(R²); R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}.

In some embodiments, R² is independently hydrogen. In some embodiments, R² is independently halogen. In some embodiments, R^{2a} is independently —CN. In some embodiments, R^{2a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments, R² is independently —OR¹². In some embodiments, R^{2a} is independently —SR¹². In some embodiments, R² is independently —N(R¹²)(R¹³). In some embodiments, R^{2a} is independently —C(O)OR¹². In some embodiments, R^{2a} is independently —OC(O)N(R¹²)(R¹³). In some embodiments, R^{2a} is independently —N(R¹⁴)C(O)N(R¹²)(R¹³). In some embodiments, R^{2a} is independently —N(R¹⁴)C(O)OR¹². In some embodiments, R^{2a} is independently —N(R¹⁴)S(O)₂R¹⁵. In some embodiments, R^{2a} is independently —C(O)R¹⁵. In some embodiments, R^{2a} is independently —S(O)R¹⁵. In some embodiments, R^{2a} is independently —OC(O)R¹⁵. In some embodiments, R^{2a} is independently —C(O)N(R¹²)(R¹³). In some embodiments, R^{2a} is independently —C(O)C(O)N(R¹²)(R¹³). In some embodiments, R^{2a} is independently —N(R¹⁴)C(O)R¹⁵. In some embodiments, R^{2a} is independently —S(O)₂R¹⁵. In some embodiments, R² is independently —S(O)₂N(R¹²)(R¹³). In some embodiments, R^{2a} is independently —S(═O)(═NH)N(R¹²)(R¹³). In some embodiments, R^{2a} is independently —CH₂C(O)N(R¹²)(R¹³). In some embodiments, R² is independently —CH₂N(R¹⁴)C(O)R¹⁵. In some embodiments, R² is independently —CH₂S(O)₂R¹⁵. In some embodiments, R^{2a} is independently —CH₂S(O)₂N(R¹²)(R¹³). In some embodiments, R^{a} is independently C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20b}. In some embodiments, R^{2a} is independently C₁₋₆alkyl substituted with one, two, or three R^{20b}. In some embodiments, R^{2a} is independently C₂₋₆alkenyl substituted with one, two, or three R^{20b}. In some embodiments, R² is independently C₂₋₆alkynyl substituted with one, two, or three R^{20b}. In some embodiments, R^{2a} is independently C₃₋₆cycloalkyl substituted with one, two, or three R^{20b}. In some embodiments, R^{2a} is independently C₂₋₉heterocycloalkyl substituted with one, two, or three R^{20b}. In some embodiments, R^{2a} is independently C₆₋₁₀aryl substituted with one, two, or three R^{20b}. In some embodiments, R^{2a} is independently C₁₋₉heteroaryl substituted with one, two, or three R^{20b}. In some embodiments, R^{2a} is independently C₁₋₆alkyl. In some embodiments, R^{2a} is independently C₂₋₆alkenyl. In some embodiments, R^{2a} is independently C₂₋₆alkynyl. In some embodiments, R^{2a} is independently C₃₋₆cycloalkyl. In some embodiments, R^{2a} is independently C₂₋₉heterocycloalkyl. In some embodiments, R^{2a} is independently C₆₋₁₀aryl. In some embodiments, R^{2a} is independently C₁₋₉heteroaryl.

In some embodiments, R^{2a} is independently —OH. In some embodiments, R^{2a} is independently —SH. In some embodiments, R^{2a} is independently —NH₂. In some embodiments, R^{2a} is independently —C(O)OH. In some embodiments, R^{2a} is independently —OC(O)NH₂. In some embodiments, R^{2a} is independently —NHC(O)NH₂. In some embodiments, R^{2a} is independently —NHC(O)OH. In some embodiments, R² is independently —NHS(O)₂R¹⁵. In some embodiments, R^{2a} is independently —C(O)R¹⁵. In some embodiments, R² is independently —S(O)R¹⁵. In some embodiments, R^{2a} is independently —OC(O)R¹⁵. In some embodiments, R^{2a} is independently —C(O)NH₂. In some embodiments, R^{2a} is independently —C(O)C(O)NH₂. In some embodiments, R² is independently —NHC(O)R¹⁵. In some embodiments, R^{2a} is independently —S(O)₂R¹⁵. In some embodiments, R² is independently —S(O)₂N(R¹²)(R¹³). In some embodiments, R^{2a} is independently —S(═O)(═NH)NH₂. In some embodiments, R^{2a} is independently —CH₂C(O)NH₂. In some embodiments, R^{2a} is independently —CH₂NHC(O)R¹⁵. In some embodiments, R² is independently —CH₂S(O)₂R¹⁵. In some embodiments, R^{2a} is independently —CH₂S(O)₂NH₂.

In some embodiments, R² is independently —NH(R¹²). In some embodiments, R^{2a} is independently —OC(O)N(R¹²)(R¹³). In some embodiments, R² is independently —NHC(O)N(R¹²). In some embodiments, R^{2a} is independently —NHC(O)OR¹². In some embodiments, R^{2a} is independently —C(O)N(R¹²)(R¹³). In some embodiments, R^{2a} is independently —C(O)C(O)NH(R¹²). In some embodiments, R^{2a} is independently —S(O)₂NH(R¹²). In some embodiments, R^{2a} is independently —S(═O)(═NH)NH(R¹²). In some embodiments, R^{2a} is independently —CH₂C(O)NH(R¹²). In some embodiments, R² is independently —CH₂S(O)₂NH(R¹²).

In some embodiments, R² and R^{2a} are combined to form a C₃₋₆cycloalkyl. In some embodiments, R² and R^{2a} are combined to form a C₂₋₉heterocycloalkyl. In some embodiments, R² and R^{2a} are combined to form a C₃₋₆cycloalkyl substituted with one, two, or three R^{20b}. In some embodiments, R² and R^{2a} are combined to form a C₂₋₉heterocycloalkyl substituted with one, two, or three R^{20b}. In some embodiments, R² and R^{2a} are combined to form a C₃ cycloalkyl. In some embodiments, R² and R² are combined to form a C₄ cycloalkyl. In some embodiments, R² and R² are combined to form a C₅ cycloalkyl. In some embodiments, R² and R^{2a} are combined to form a C₆ cycloalkyl. In some embodiments, R² and R² are combined to form a C₂heterocycloalkyl. In some embodiments, R² and R² are combined to form a C₃ heterocycloalkyl. In some embodiments, R² and R^{2a} are combined to form a C₄ heterocycloalkyl. In some embodiments, R² and R^{2a} are combined to form a C₅ heterocycloalkyl. In some embodiments, R² and R^{2a} are combined to form a C₆ heterocycloalkyl. In some embodiments, R² and R^{2a} are combined to form a C₇ heterocycloalkyl. In some embodiments, R² and R^{2a} are combined to form a C₈ heterocycloalkyl. In some embodiments, R² and R² are combined to form a C₉ heterocycloalkyl.

In some embodiments, R² and R^{2a} are combined to form a C₃ heterocycloalkyl substituted with one, two, or three R^{20b}. In some embodiments, R² and R^{2a} are combined to form a C₄ heterocycloalkyl substituted with one, two, or three R^{20b}. In some embodiments, R² and R^{2a} are combined to form a C₅ heterocycloalkyl substituted with one, two, or three R^{20b}. In some embodiments, R² and R^{2a} are combined to form a C₆ heterocycloalkyl substituted with one, two, or three R^{20b}. In some embodiments, R² and R^{2a} are combined to form a C₇ heterocycloalkyl substituted with one, two, or three R^{20b}. In some embodiments, R² and R^{2a} are combined to form a C₈ heterocycloalkyl substituted with one, two, or three R^{20b}. In some embodiments, R² and R^{2a} are combined to form a C₉ heterocycloalkyl substituted with one, two, or three R^{20b}.

In some embodiments, R² is selected from hydrogen, halogen, C₁₋₆alkyl, C₂₋₉heterocycloalkyl, C₁₋₉heteroaryl, —OR¹², —SR¹², —N(R¹²)(R¹³), —C(O)R¹⁵, —C(O)N(R¹²)(R¹³), —S(O)₂R¹⁵, and —S(O)₂N(R¹²)(R¹³)—, wherein C₁₋₆alkyl, C₂₋₉heterocycloalkyl, and C₁₋₉ heteroaryl are optionally substituted with one, two, or three R^{20b}.

In some embodiments, R² is selected from C₁₋₆alkyl, C₂₋₉heterocycloalkyl, C₁₋₉heteroaryl, —OR¹², —N(R¹²)(R¹³), —C(O)R¹⁵, —C(O)N(R¹²)(R¹³), wherein C₁₋₆alkyl, C₂₋₉heterocycloalkyl, and C₁₋₉heteroaryl are optionally substituted with one, two, or three R^{20b}.

In some embodiments, R² is —OR¹². In some embodiments, R² is —N(R¹²)(R¹³). In some embodiments, R² is —C(O)R¹⁵. In some embodiments, R² is —C(O)N(R¹²)(R¹³). In some embodiments, R² is —OR¹². In some embodiments, R² is —NH(R¹²). In some embodiments, R² is —C(O)R¹⁵. In some embodiments, R² is —C(O)NH(R¹²). In some embodiments, R¹² is C₁-C₆alkyl. In some embodiments, R¹² is C₁-C₆alkyl substituted with one R^{20k}. In some embodiments, R¹² is C₁alkyl substituted with one R^{20k}. In some embodiments, R¹² is C₂alkyl substituted with one R^{20k}. In some embodiments, R¹² is C₃alkyl substituted with one R^{20k}. In some embodiments, R¹² is C₄alkyl substituted with one R^{20k}. In some embodiments, R¹² is C₅alkyl substituted with one R^{20k}. In some embodiments, R¹² is C₆alkyl substituted with one R^{20k}. In some embodiments, R¹² is C₂₋₉heterocycloalkyl substituted with one R^{20k}. In some embodiments, R¹² is C₄heterocycloalkyl substituted with one R^{20k}. In some embodiments, R¹² is C₃heterocycloalkyl substituted with one R^{20k}. In some embodiments, R¹² is C₃heterocycloalkyl. In some embodiments, R¹² is C₁-C₆alkyl substituted with two R^{20k}. In some embodiments, R¹⁵ is C₁-C₆alkyl. In some embodiments, R¹⁵ is C₁-C₆alkyl substituted with one R^{20k}. In some embodiments, R¹⁵ is C₁alkyl substituted with one R^{20k}. In some embodiments, R¹⁵ is C₂alkyl substituted with one R^{20k}. In some embodiments, R¹⁵ is C₃alkyl substituted with one R^{20k}. In some embodiments, R¹⁵ is C₄alkyl substituted with one R^{20k}. In some embodiments, R¹⁵ is C₅alkyl substituted with one R^{20k}. In some embodiments, R¹⁵ is C₆alkyl substituted with one R^{20k}. In some embodiments, R¹⁵ is C₂₋₉heterocycloalkyl substituted with one R^{20k}. In some embodiments, R¹⁵ is C₄heterocycloalkyl substituted with one R^{20k}. In some embodiments, R¹⁵ is C₃heterocycloalkyl substituted with one R^{20k}. In some embodiments, R¹⁵ is C₃heterocycloalkyl. In some embodiments, R¹⁵ is C₁-C₆alkyl substituted with two R^{20k}. In some embodiments, R^{20k} is independently —NHS(O)₂R²⁵ and R²⁵ is C₃₋₆cycloalkyl. In some embodiments, R^{20k} is independently —S(O)₂R²⁵ and R²⁵ is C₁₋₆alkyl. In some embodiments, R^{20k} is —OR²¹ and R²¹ is C₃₋₆cycloalkyl. In some embodiments, R^{20k} is —CF₃. In some embodiments, R^{20k} is —C(halogen)₃. In some embodiments, R^{20k} is independently halogen. In some embodiments, R^{20k} is independently —F. In some embodiments, R^{20k} is independently C₃₋₆cycloalkyl. In some embodiments, R^{20k} is —OR²¹ and R²¹ is C₁₋₆alkyl. In some embodiments, R^{20k} is —CN. In some embodiments, R^{20k} is C₆₋₁₀aryl. In some embodiments, R^{20k} is phenyl. In some embodiments, R^{20k} is C₂₋₉heterocycloalkyl. In some embodiments, R^{20k} is C₅heterocycloalkyl. In some embodiments, R^{20k} is C₅heterocycloalkyl substituted with C₁₋₆alkyl.

In some embodiments, R² is C₁₋₉heteroaryl substituted with one R^{20k}. In some embodiments, R² is C₃heteroaryl substituted with one R^{20b}. In some embodiments, R² is C₂₋₉heterocycloalkyl. In some embodiments, R² is C₄heterocycloalkyl. In some embodiments, R² is C₂₋₉heterocycloalkyl substituted with one R^{20b}. In some embodiments, R² is —N(R¹⁴)C(O)R¹⁵. In some embodiments, R² is C₂₋₆alkynyl. In some embodiments, R^{20b} is independently —NHS(O)₂R²⁵ and R²⁵ is C₃₋₆cycloalkyl. In some embodiments, R^{20b} is independently —S(O)₂R²⁵ and R²⁵ is C₁₋₆alkyl. In some embodiments, R^{20b} is —OR²¹ and R²¹ is C₃₋₆cycloalkyl. In some embodiments, R^{20b} is —CF₃. In some embodiments, R^{20b} is —C(halogen)₃. In some embodiments, R^{20b} is independently halogen. In some embodiments, R^{20b} is independently —F. In some embodiments, R^{20b} is independently C₃₋₆cycloalkyl. In some embodiments, R^{20b} is —OR²¹ and R²¹ is C₁₋₆alkyl. In some embodiments, R^{20b} is —CN. In some embodiments, R^{20b} is C₆₋₁₀aryl. In some embodiments, R^{20b} is phenyl. In some embodiments, R^{20b} is C₂₋₉heterocycloalkyl. In some embodiments, R^{20b} is C₅heterocycloalkyl. In some embodiments, R^{20b} is C₅heterocycloalkyl substituted with C₁₋₆alkyl. In some embodiments, R² is C₁₋₆alkyl substituted with one R^{20b} In some embodiments, R^{20b} is C₁-C₆alkyl. In some embodiments, R^{20b} is C₁-C₆alkyl substituted with C₁-C₆alkyl substituted with —NHS(O)₂R²⁵ and R²⁵ is C₃₋₆cycloalkyl. In some embodiments, R^{20b} is C₁-C₆alkyl substituted with C₂₋₉heterocycloalkyl substituted with —S(O)₂R²⁵ and R²⁵ is C₁₋₆alkyl. In some embodiments, R^{20b} is C₁-C₆alkyl substituted with C₄heterocycloalkyl substituted with —S(O)₂R²⁵ and R²⁵ is C₁₋₆alkyl. In some embodiments, R^{20b} is C₁-C₆alkyl substituted with C₃heterocycloalkyl substituted with —S(O)₂R²⁵ and R²⁵ is C₁₋₆alkyl. In some embodiments, R^{20b} is C₁-C₆alkyl substituted with C₃heterocycloalkyl. In some embodiments, R^{20b} is C₁-C₆alkyl substituted with C₁-C₆alkyl substituted with two halogen. In some embodiments, R^{20b} is C₁-C₆alkyl substituted with C₁-C₆alkyl substituted with —OR²¹ and R²¹ is C₃₋₆cycloalkyl. In some embodiments, R^{20b} is C₁-C₆alkyl substituted with C₁-C₆alkyl substituted with —CF₃. In some embodiments, R^{20b} is C₁-C₆alkyl substituted with C₁-C₆alkyl substituted with —C(halogen)₃. In some embodiments, R^{20b} is C₁-C₆alkyl substituted with C₁-C₆alkyl substituted with halogen. In some embodiments, R^{20b} is C₁-C₆alkyl substituted with C₁-C₆alkyl substituted with —F. In some embodiments, R^{20b} is C₁-C₆alkyl substituted with C₁-C₆alkyl substituted with C₃₋₆cycloalkyl. In some embodiments, R^{20b} is C₁-C₆alkyl substituted with C₁-C₆alkyl substituted with —OR²¹ and R²¹ is C₁₋₆alkyl. In some embodiments, R^{20b} is C₁-C₆alkyl substituted with C₁-C₆alkyl substituted with —CN. In some embodiments, R^{20b} is C₁-C₆alkyl substituted with C₁-C₆alkyl substituted with C₆₋₁₀aryl. In some embodiments, R^{20b} is C₁-C₆alkyl substituted with C₁-C₆alkyl substituted with phenyl. In some embodiments, R^{20b} is C₁-C₆alkyl substituted with C₁-C₆alkyl substituted with C₂₋₉heterocycloalkyl. In some embodiments, R^{20b} is C₁-C₆alkyl substituted with C₁-C₆alkyl substituted with C₅heterocycloalkyl. In some embodiments, R^{20b} is C₁-C₆alkyl substituted with C₁-C₆alkyl substituted with C₅heterocycloalkyl substituted with C₁₋₆alkyl.

In some embodiments, R² is selected from —OR¹², —N(R¹²)(R¹³), —C(O)R¹⁵, and —C(O)N(R¹²)(R¹³); R¹³ is hydrogen; R¹² is independently

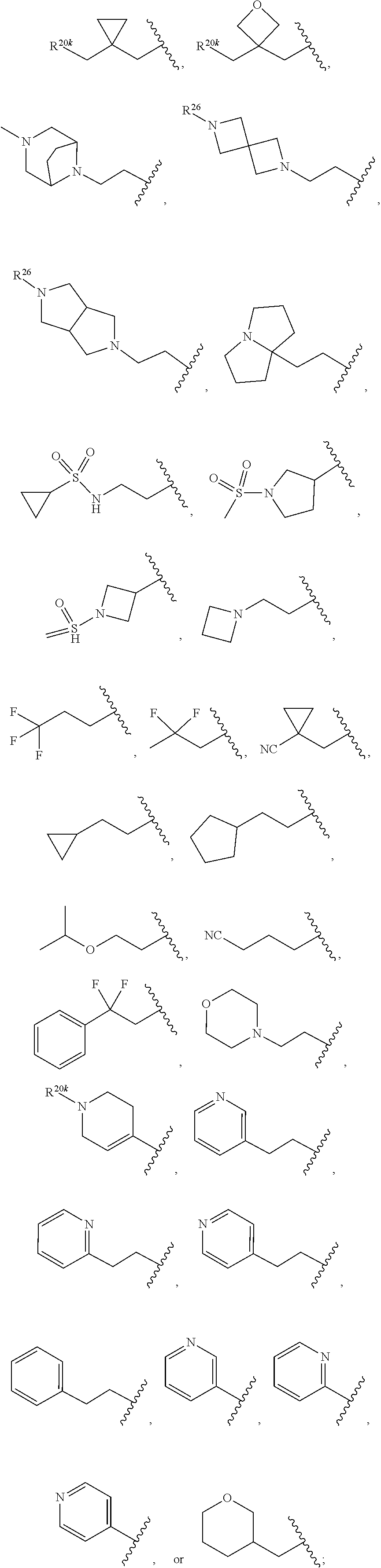

R¹⁵ is independently

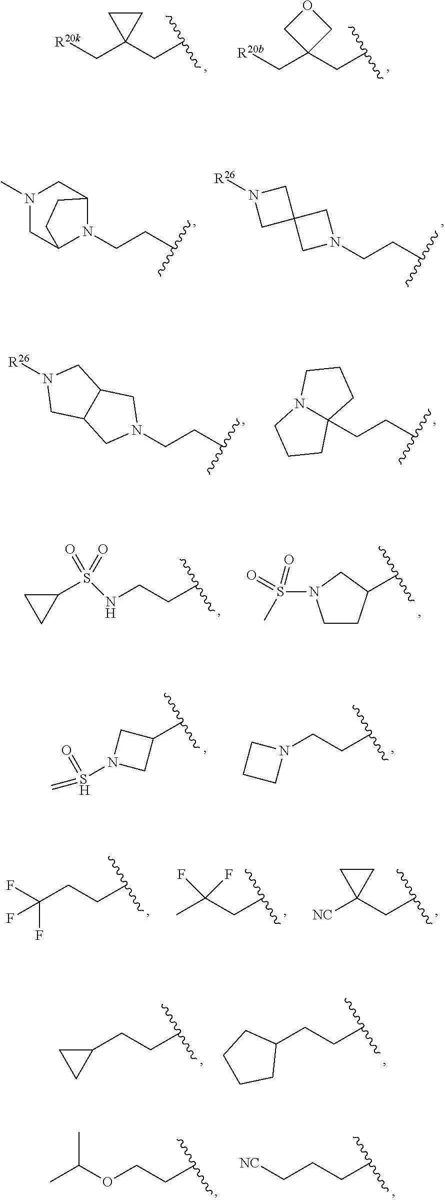

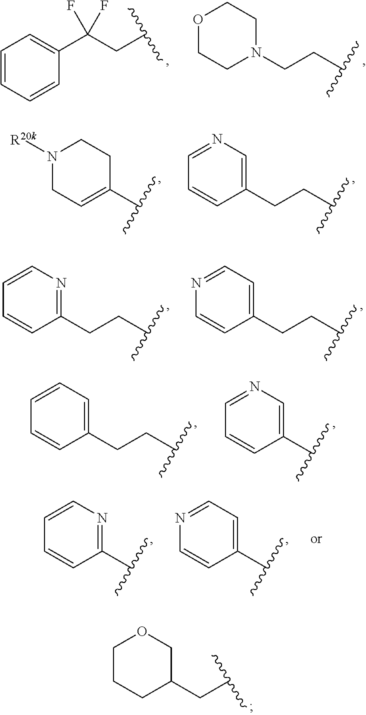

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, R² is selected from —OR¹², —N(R¹²)(R¹³), —C(O)R¹⁵, and —C(O)N(R¹²)(R¹³); R¹³ is hydrogen; R¹² is independently

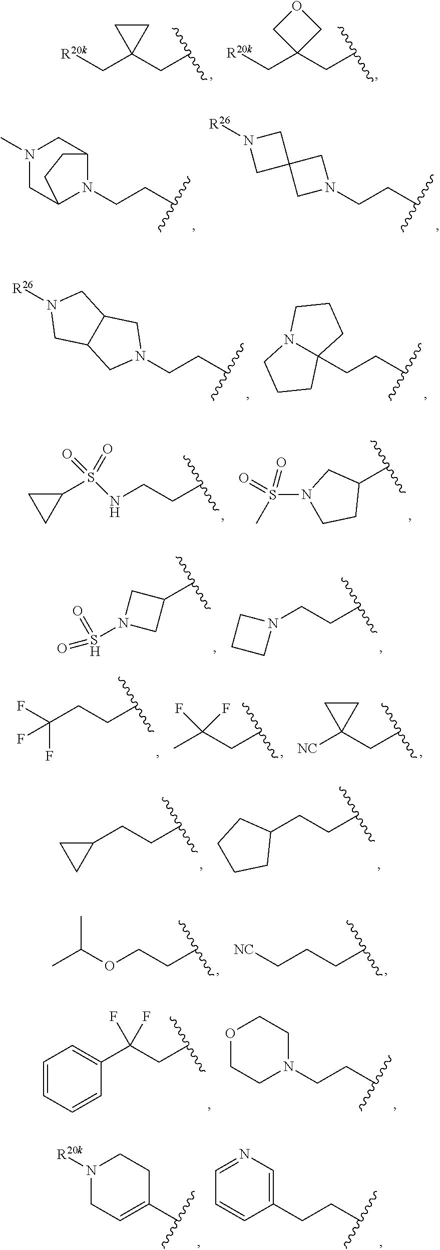

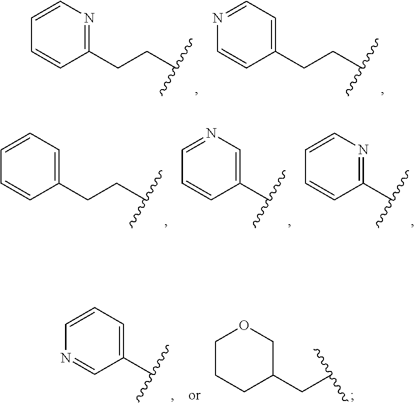

R¹⁵ is independently

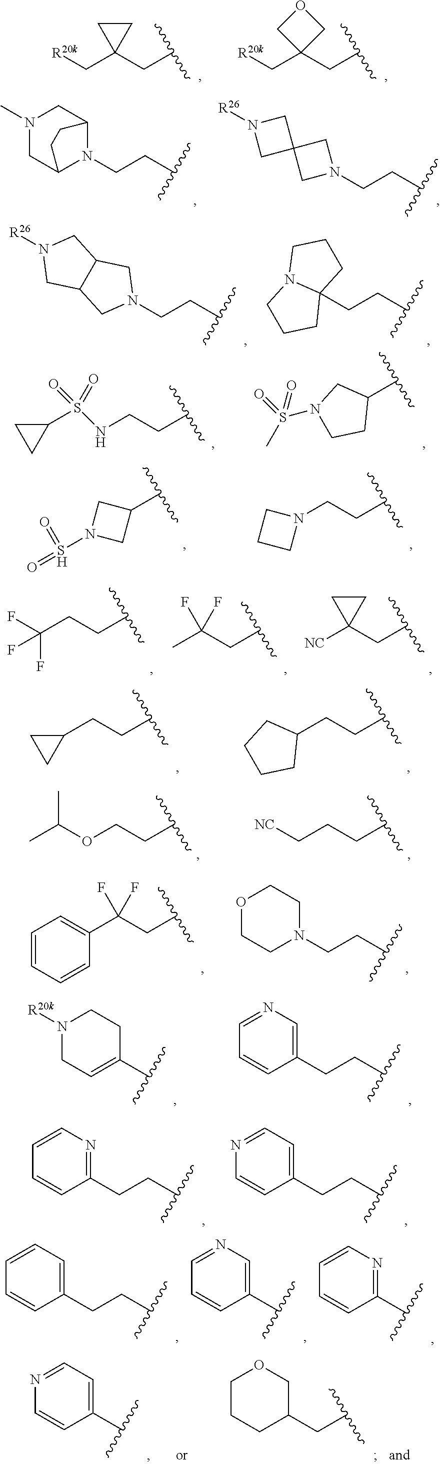

R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, R² is selected from

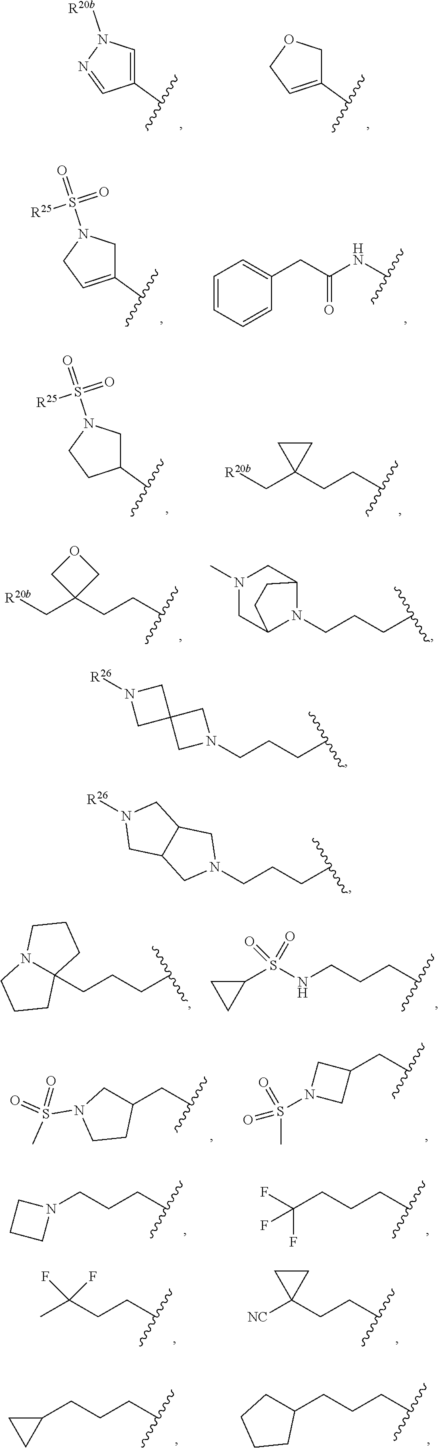

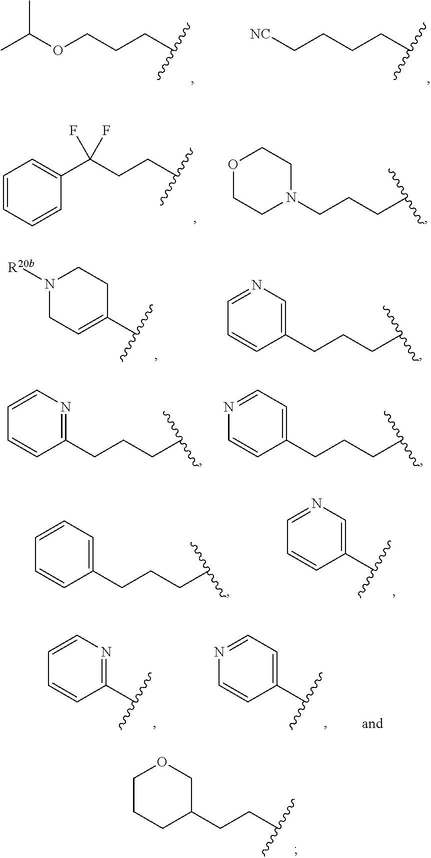

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, R² is selected from —OR¹², —N(R¹²)(R¹³), —C(O)R¹⁵, and —C(O)N(R¹²)(R¹³); R¹³ is hydrogen; R¹² and R¹⁵ are independently

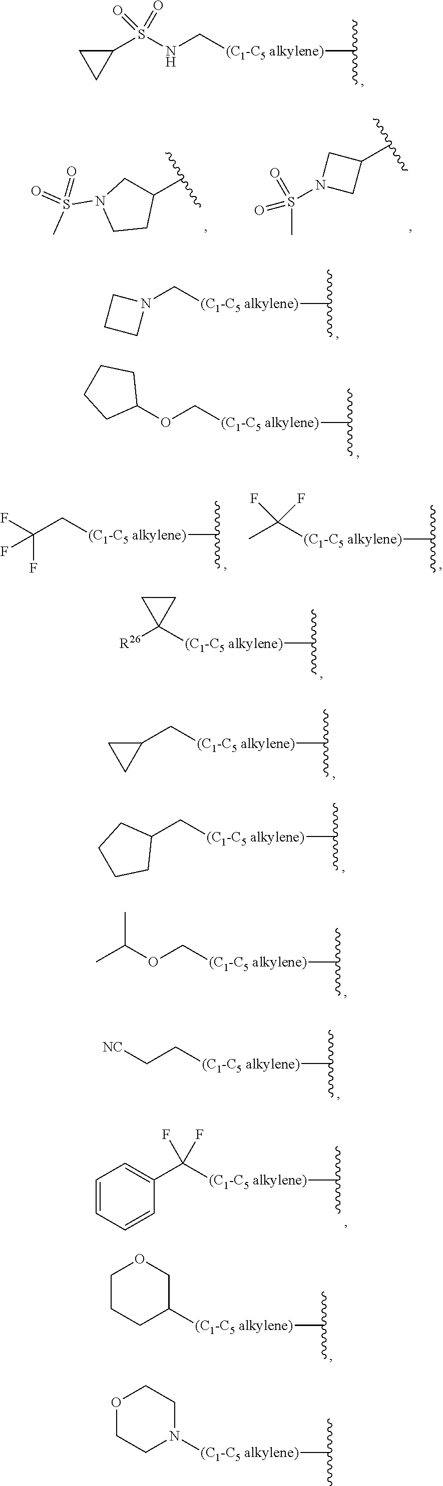

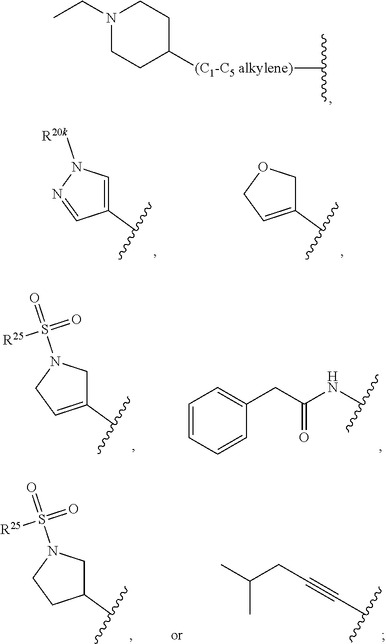

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³) C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O₂R²⁵, —C(O)R²⁵, —S(O)₂R²⁵, —S(O)₂N(R²²)(R²³), and —OC(O)R²⁵.

In some embodiments, R² is independently

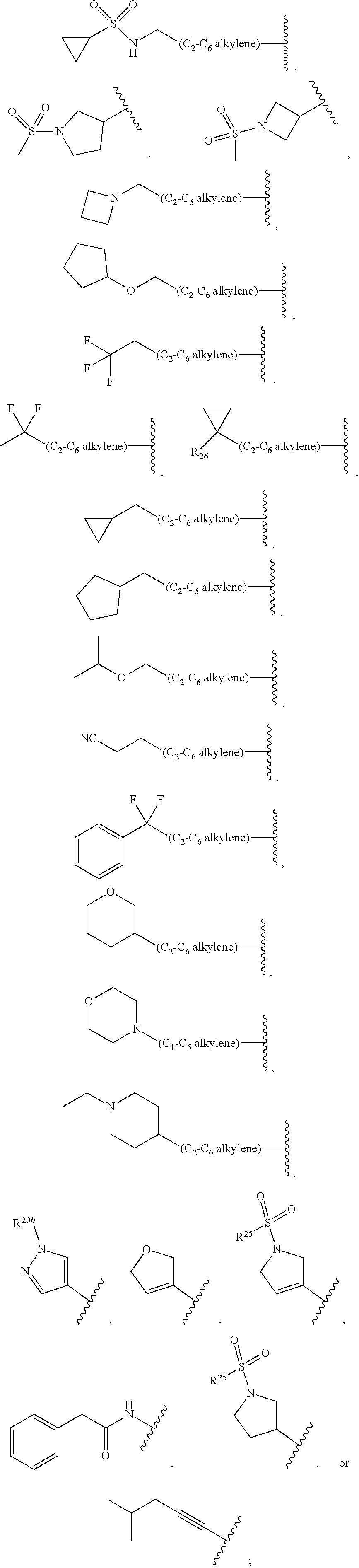

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is

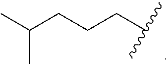 [SMILES: *CCCC(C)C]

In some embodiments, R² is

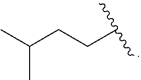 [SMILES: *CCC(C)C]

In some embodiments, R² is

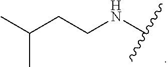 [SMILES: *NCCC(C)C]

In some embodiments, R² is

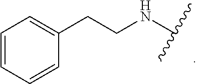 [SMILES: *NCCc1ccccc1]

In some embodiments, R² is

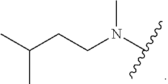 [SMILES: *N(C)CCC(C)C]

In some embodiments, R² is

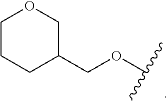 [SMILES: *OCC1CCCOC1]

In some embodiments, R² is

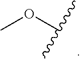 [SMILES: *OC]

In some embodiments, R² is —CN. In some embodiments, R² is —F. In some embodiments, R² is —CF₃. In some embodiments, R² is —OCH₃. In some embodiments, R² is cyclopropyl. In some embodiments, R² is —OCH₂CF₃. In some embodiments, R² is —CH₃.

In some embodiments (e.g., including Formulae (I), (II), (III), (IV); or sub-formulae thereof), W³ is N. In some embodiments (e.g., including Formulae (I), (II), (III), (IV); or sub-formulae thereof), W³ is N(R³). In some embodiments (e.g., including Formulae (I), (II), (III), (IV); or sub-formulae thereof), W³ is O. In some embodiments (e.g., including Formulae (I), (II), (III), (IV); or sub-formulae thereof), W³ is C(R³). In some embodiments (e.g., including Formulae (I), (II), (III), (IV); or sub-formulae thereof), W³ is C(R³)(R^{3a}). In some embodiments (e.g., including Formulae (I), (II), (III), (IV); or sub-formulae thereof), W³ is C(O). In some embodiments (e.g., including Formulae (I), (II), (III), (IV); or sub-formulae thereof), W³ is S(O). In some embodiments (e.g., including Formulae (I), (II), (III), (IV); or sub-formulae thereof), W³ is S(O)₂. In some embodiments (e.g., including Formulae (I), (II), (III), (IV); or sub-formulae thereof), W³ is S(O)(NR³). In some embodiments (e.g., including Formulae (I), (II), (III), (IV); or sub-formulae thereof), W³ is S(O)(R³). In some embodiments (e.g., including Formulae (I), (II), (III), (IV); or sub-formulae thereof), W³ is P(O)(R³).

In some embodiments (e.g., including Formulae (I), (II), (III), (IV); or sub-formulae thereof), R³ is independently hydrogen. In some embodiments (e.g., including Formulae (I), (II), (III), (IV); or sub-formulae thereof), R³ is independently halogen. In some embodiments (e.g., including Formulae (I), (II), (III), (IV); or sub-formulae thereof), R³ is independently —CN. In some embodiments (e.g., including Formulae (I), (II), (III), (IV); or sub-formulae thereof), R³ is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments (e.g., including Formulae (I), (II), (III), (IV); or sub-formulae thereof), R³ is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NIH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R³ is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20c}.

In some embodiments, R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments, when W³ is N(R³), S(O)(NR³), S(O)(R³), or P(O)(R³); R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —C(O)OR¹², —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20c}. In some embodiments, R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments, when W³ is N(R³), S(O)(NR³), S(O)(R³), or P(O)(R³); R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}.

In some embodiments, R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments, R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments, when W³ is N(R³), S(O)(NR³), S(O)(R³), or P(O)(R³); R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R². In some embodiments, when W³ is N(R³), S(O)(NR³), S(O)(R³), or P(O)(R³); R³ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}.

In some embodiments (e.g., including Formulae (I), (II), (III), (IV); or sub-formulae thereof), R^{3a} is independently hydrogen. In some embodiments (e.g., including Formulae (I), (II), (III), (IV); or sub-formulae thereof), R^{3a} is independently halogen. In some embodiments (e.g., including Formulae (I), (II), (III), (IV); or sub-formulae thereof), R^{3a} is independently —CN. In some embodiments (e.g., including Formulae (I), (II), (III), (IV); or sub-formulae thereof), R^{3a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments (e.g., including Formulae (I), (II), (III), (IV); or sub-formulae thereof), R^{3a} is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R^{3a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20c}.

In some embodiments (e.g., including Formulae (I), (II), (III), (IV); or sub-formulae thereof), R³ and R^{3a} are combined to form a C₃₋₆cycloalkyl. In some embodiments (e.g., including Formulae (I), (II), (III), (IV); or sub-formulae thereof), R³ and R^{3a} are combined to form a C₂₋₉heterocycloalkyl. In some embodiments (e.g., including Formulae (I), (II), (III), (IV); or sub-formulae thereof), R³ and R^{3a} are combined to form a C₃₋₆cycloalkyl substituted with one, two, or three R^{20c}. In some embodiments (e.g., including Formulae (I), (II), (III), (IV); or sub-formulae thereof), R³ and R^{3a} are combined to form a C₂₋₉heterocycloalkyl substituted with one, two, or three R^{20c}.

In some embodiments (e.g., including Formulae (I), (II), (III), (IV); or sub-formulae thereof), R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}.

In some embodiments (e.g., including Formulae (I), (II), (III), (IV); or sub-formulae thereof), R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}. In some embodiments, R³ is —CN. In some embodiments, R³ is —F. In some embodiments, R³ is —CF₃. In some embodiments, R³ is —OCH₃. In some embodiments, R³ is cyclopropyl. In some embodiments, R³ is —OCH₂CF₃. In some embodiments, R³ is —CH₃.

In some embodiments (e.g., including Formulae (I), (II), (III), (IV); or sub-formulae thereof), W⁴ is N. In some embodiments (e.g., including Formulae (I), (II), (III), (IV); or sub-formulae thereof), W⁴ is N(R⁴). In some embodiments (e.g., including Formulae (I), (II), (III), (IV); or sub-formulae thereof), W⁴ is O. In some embodiments (e.g., including Formulae (I), (II), (III), (IV); or sub-formulae thereof), W⁴ is C(R⁴). In some embodiments (e.g., including Formulae (I), (II), (III), (IV); or sub-formulae thereof), W⁴ is C(R⁴)(R^{4a}). In some embodiments (e.g., including Formulae (I), (II), (III), (IV); or sub-formulae thereof), W⁴ is C(O). In some embodiments (e.g., including Formulae (I), (II), (III), (IV); or sub-formulae thereof), W⁴ is S(O). In some embodiments (e.g., including Formulae (I), (II), (III), (IV); or sub-formulae thereof), W⁴ is S(O)₂. In some embodiments (e.g., including Formulae (I), (II), (III), (IV); or sub-formulae thereof), W⁴ is S(O)(NR⁴). In some embodiments (e.g., including Formulae (I), (II), (III), (IV); or sub-formulae thereof), W⁴ is S(O)(R⁴). In some embodiments (e.g., including Formulae (I), (II), (III), (IV); or sub-formulae thereof), W⁴ is P(O)(R⁴).

In some embodiments (e.g., including Formulae (I), (II), (III), (IV); or sub-formulae thereof), R⁴ is independently hydrogen. In some embodiments (e.g., including Formulae (I), (II), (III), (IV); or sub-formulae thereof), R⁴ is independently halogen. In some embodiments (e.g., including Formulae (I), (II), (III), (IV); or sub-formulae thereof), R⁴ is independently —CN. In some embodiments (e.g., including Formulae (I), (II), (III), (IV); or sub-formulae thereof), R⁴ is independently C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (I), (II), (III), (IV); or sub-formulae thereof), R⁴ is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R⁴ is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20d}. In some embodiments (e.g., including Formulae (I), (II), (III), (IV); or sub-formulae thereof), R⁴ is independently C₁₋₆alkyl. In some embodiments (e.g., including Formulae (I), (II), (III), (IV); or sub-formulae thereof), R⁴ is independently C₁₋₄alkyl. In some embodiments (e.g., including Formulae (I), (II), (III), (IV); or sub-formulae thereof), R⁴ is independently C₁₋₃alkyl. In some embodiments (e.g., including Formulae (I), (II), (III), (IV); or sub-formulae thereof), R⁴ is independently C₁₋₂alkyl. In some embodiments (e.g., including Formulae (I), (II), (III), (IV); or sub-formulae thereof), R⁴ is independently methyl. In some embodiments, R⁴ is —CN. In some embodiments, R⁴ is —F. In some embodiments, R⁴ is —CF₃. In some embodiments, R⁴ is —OCH₃. In some embodiments, R⁴ is cyclopropyl. In some embodiments, R⁴ is —OCH₂CF₃. In some embodiments, R⁴ is —CH₃.

In some embodiments, R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}. In some embodiments, R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}. In some embodiments, when W⁴ is N(R⁴), S(O)(NR⁴), S(O)(R⁴), or P(O)(R⁴). R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}. In some embodiments, when W⁴ is N(R⁴), S(O)(NR⁴), S(O)(R⁴), or P(O)(R⁴); R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}.

In some embodiments (e.g., including Formulae (I), (II), (III), (IV); or sub-formulae thereof), R^{4a} is independently hydrogen. In some embodiments (e.g., including Formulae (I), (II), (III), (IV); or sub-formulae thereof), R^{4a} is independently halogen. In some embodiments (e.g., including Formulae (I), (II), (III), (IV); or sub-formulae thereof), R^{4a} is independently —CN. In some embodiments (e.g., including Formulae (I), (II), (III), (IV); or sub-formulae thereof), R^{4a} is independently C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (I), (II), (III), (IV); or sub-formulae thereof), R^{4a} is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (I), (II), (III), (IV); or sub-formulae thereof), R^{4a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20d}. In some embodiments (e.g., including Formulae (I), (II), (III), (IV); or sub-formulae thereof), R^{4a} is independently C₁₋₆alkyl. In some embodiments (e.g., including Formulae (I), (II), (III), (IV); or sub-formulae thereof), R^{4a} is independently C₁₋₄alkyl. In some embodiments (e.g., including Formulae (I), (II), (III), (IV); or sub-formulae thereof), R^{4a} is independently C₁₋₃alkyl. In some embodiments (e.g., including Formulae (I), (II), (III), (IV); or sub-formulae thereof), R^{4a} is independently C₁₋₂alkyl. In some embodiments (e.g., including Formulae (I), (II), (III), (IV); or sub-formulae thereof), R^{4a} is independently methyl.

In some embodiments (e.g., including Formulae (I), (II), (III), (IV); or sub-formulae thereof), R⁴ and R^{4a} are combined to form a C₃₋₆cycloalkyl. In some embodiments (e.g., including Formulae (I), (II), (III), (IV); or sub-formulae thereof), R⁴ and R^{4a} are combined to form a C₂₋₉heterocycloalkyl. In some embodiments (e.g., including Formulae (I), (II), (III), (IV); or sub-formulae thereof), R⁴ and R^{4a} are combined to form a C₃₋₆cycloalkyl substituted with one, two, or three R^{20d}. In some embodiments (e.g., including Formulae (I), (II), (III), (IV); or sub-formulae thereof), R⁴ and R^{4a} are combined to form a C₂₋₉heterocycloalkyl substituted with one, two, or three R^{20d}.

In some embodiments (e.g., including Formulae (I), (II), (III), (IV); or sub-formulae thereof), W⁵ is N. In some embodiments (e.g., including Formulae (I), (II), (III), (IV); or sub-formulae thereof), W⁵ is C.

In some embodiments (e.g., including Formulae (I), (II), (III), (IV); or sub-formulae thereof), W⁶ is N. In some embodiments (e.g., including Formulae (I), (II), (III), (IV); or sub-formulae thereof), W⁶ is C.

In some embodiments (e.g., including Formulae (I), (II), (III), (IV); or sub-formulae thereof), at least one of W¹, W², W³, W⁴, W⁵, and W⁶ is N, N(R¹), N(R²), N(R³), or N(R⁴). In some embodiments (e.g., including Formulae (I), (II), (III), (IV); or sub-formulae thereof), at least one of W¹, W², W³, and W⁴ is N, N(R¹), N(R²), N(R³), or N(R⁴).

In some embodiments (e.g., including Formulae (I); or sub-formulae thereof), two Ring A [symbol] are double bonds. In some embodiments (e.g., including Formulae (I); or sub-formulae thereof), three Ring A [symbol] are double bonds. In some embodiments of Formulae (III), or sub-formulae thereof, no Ring A [symbol] are double bonds. In some embodiments of Formulae (III), or sub-formulae thereof, one Ring A [symbol] is a double bond. In some embodiments of Formulae (III), or sub-formulae thereof, two Ring A [symbol] are double bonds.

In some embodiments of Formula (III), or sub-formulae thereof, W³ is C(O), S(O), S(O)₂, S(O)(NR³), S(O)(R³), or P(O)(R³) W⁴ is N(R⁴); W¹ is C(R¹); and W² is C(R²). In some embodiments of Formula (III), or sub-formulae thereof, W¹ is C(O), S(O), S(O)₂, S(O)(NR¹), S(O)(R¹), or P(O)(R¹); W² is N(R²); W³ is C(R³)(R^{3a}); and W⁴ is C(R⁴)(R^{4a}). In some embodiments of Formula (III), or sub-formulae thereof, W³ is C(O), S(O), S(O)₂, S(O)(NR³), S(O)(R³), or P(O)(R³); W⁴ is N(R⁴); W² is N(R²); and W¹ is C(R¹)(R^{1a}). In some embodiments of Formula (III), or sub-formulae thereof, W³ is C(O), S(O), S(O)₂, S(O)(NR³), S(O)(R³), or P(O)(R³); W² is N(R²); W¹ is C(R¹)(R^{1a}); and W⁴ is C(R⁴)(R^{4a}). In some embodiments of Formula (III), or sub-formulae thereof, W³ is C(O), S(O), S(O)₂, S(O)(NR³), S(O)(R³), or P(O)(R³); W² is N(R²); W¹ is C(R¹); and W⁴ is N. In some embodiments of Formula (III), or sub-formulae thereof, W³ is C(O), S(O), S(O)₂, S(O)(NR³), S(O)(R³), or P(O)(R³); W² is N(R²); W¹ is C(R¹); and W⁴ is C(R⁴). In some embodiments of Formula (III), or sub-formulae thereof, W⁴ is C(O), S(O), S(O)₂, S(O)(NR⁴), S(O)(R⁴), or P(O)(R⁴); W³ is N(R³); W¹ is N; and W² is C(R²). In some embodiments of Formula (III), or sub-formulae thereof, W⁴ is C(O), S(O), S(O)₂, S(O)(NR⁴), S(O)(R), or P(O)(R⁴); W³ is N(R³); W¹ is C(R¹); and W² is C(R²). In some embodiments of Formula (III), or sub-formulae thereof, W³ is C(O), S(O), S(O)₂, S(O)(NR³), S(O)(R³), or P(O)(R³); W⁴ is N(R⁴); W² is C(R²); and W¹ is C(R¹). In some embodiments of Formula (III), or sub-formulae thereof, W¹ is C(O), S(O), S(O)₂, S(O)(NR¹), S(O)(R¹), or P(O)(R¹); W² is N(R²); W³ is C(R³); and W⁴ is C(R⁴). In some embodiments of Formula (III), or sub-formulae thereof, W¹ is C(O), S(O), S(O)₂, S(O)(NR¹), S(O)(R¹), or P(O)(R¹); W² is N(R²); W³ is C(R³); and W⁴ is N. In some embodiments of Formula (III), or sub-formulae thereof, W⁴ is C(O), S(O), S(O)₂, S(O)(NR⁴), S(O)(R⁴), or P(O)(R⁴); W³ is C(R³)(R^{3a}); W¹ is O; and W² is C(R²)(R^{2a}). In some embodiments of Formula (III), or sub-formulae thereof, W⁴ is C(O), S(O), S(O)₂, S(O)(NR⁴), S(O)(R⁴), or P(O)(R⁴); W³ is C(R³); W¹ is O; and W² is C(R²). In some embodiments of Formula (III), or sub-formulae thereof, W¹ is C(O), S(O), S(O)₂, S(O)(NR¹), S(O)(R¹), or P(O)(R¹); W³ is C(R³)(R^{3a}); W⁴ is O; and W² is C(R²)(R^{2a}). In some embodiments of Formula (III), or sub-formulae thereof, W¹ is C(O), S(O), S(O)₂, S(O)(NR¹), S(O)(R¹), or P(O)(R¹); W³ is C(R³); W⁴ is O; and W² is C(R²).

In some embodiments (e.g., including Formulae (I), (II), (III), (IV); or sub-formulae thereof), W¹ is C(R¹); W² is C(R²); W³ is N; and W⁴ is N. In some embodiments (e.g., including Formulae (I), (II), (III), (IV); or sub-formulae thereof), W¹ is C(R¹); W² is C(R²); W³ is C(R³); and W⁴ is N. In some embodiments (e.g., including Formulae (I), (II), (III), (IV); or sub-formulae thereof), W¹ is C(R¹); W² is C(R²); W³ is N; and W⁴ is C(R⁴). In some embodiments (e.g., including Formulae (I), (II), (III), (IV); or sub-formulae thereof), W¹ is N; W² is C(R²); W³ is C(R³); and W⁴ is C(R⁴).

In some embodiments (e.g., including Formulae (I), (II), (III), (IV); or sub-formulae thereof), W¹ is N, W² is C(R²), W³ is C(R³), and W⁴ is C(R⁴). In some embodiments (e.g., including Formulae (I), (II), (III), (IV); or sub-formulae thereof), W¹ is C(R¹), W² is N, W³ is C(R³), and W⁴ is C(R⁴). In some embodiments (e.g., including Formulae (I), (II), (III), (IV); or sub-formulae thereof), W¹ is C(R¹), W² is C(R²), W³ is N, and W⁴ is C(R⁴). In some embodiments (e.g., including Formulae (I), (II), (III), (IV); or sub-formulae thereof), W¹ is C(R¹), W² is C(R²), W³ is C(R³), and W⁴ is N. In some embodiments (e.g., including Formulae (I), (II), (III), (IV); or sub-formulae thereof), W¹ is C(R¹) and W² is C(R²). In some embodiments (e.g., including Formulae (I), (II), (III), (IV); or sub-formulae thereof), W³ is N(R³) and W⁴ is C(O). In some embodiments (e.g., including Formulae (I), (II), (III), (IV); or sub-formulae thereof), W³ is C(O) and W⁴ is N(R⁴). In some embodiments (e.g., including Formulae (I), (II), (III), (IV); or sub-formulae thereof), W³ is O and W⁴ is C(R⁴)(R^{4a}). In some embodiments (e.g., including Formulae (I), (II), (III), (IV); or sub-formulae thereof), W¹ is C(R¹)(R^{1a}), W² is O, W³ is C(R³), and W⁴ is C(R⁴). In some embodiments (e.g., including Formulae (I), (II), (III), (IV); or sub-formulae thereof), W¹ is CO, N(R¹) or C(R¹)(R^{1a}); W² is CO, N(R²), or C(R²)(R^{2a}); W³ is N, or C(R³); and W⁴ is N, or C(R⁴). In some embodiments (e.g., including Formulae (I), (II), (III), (IV); or sub-formulae thereof), W³ and W⁴ are connected by a double bond. In some embodiments (e.g., including Formulae (I), (II), (III), (IV); or sub-formulae thereof), W¹ is is N(R¹) and W³ and W⁴ are connected by a double bond. In some embodiments (e.g., including Formulae (I), (II), (III), (IV); or sub-formulae thereof), W² is N(R²) and W¹ is C(R¹)(R^{1a}). In some embodiments (e.g., including Formulae (I), (II), (III), (IV); or sub-formulae thereof), W⁴ is N(R⁴) and W¹ and W² are connected by a double bond. In some embodiments (e.g., including Formulae (I), (II), (III), (IV); or sub-formulae thereof), W² is C(R²)(R²) and W³ is C(R³)(R^{3a}). In some embodiments (e.g., including Formulae (I), (II), (III), (IV); or sub-formulae thereof), W² is C(R²) and W³ is C(R³). In some embodiments of Formula (III), or sub-formulae thereof, W⁴ is C(O), S(O), S(O)₂, S(O)(NR⁴), S(O)(R⁴), or P(O)(R⁴); W³ is N; and W² is C(R²). In some embodiments of Formula (III), or sub-formulae thereof, W⁴ is C(O), S(O), S(O)₂, S(O)(NR⁴), S(O)(R⁴), or P(O)(R⁴); W² is N; and W³ is C(R³). In some embodiments of Formula (III), or sub-formulae thereof, W⁴ is C(O), S(O), S(O)₂, S(O)(NR⁴), S(O)(R⁴), or P(O)(R⁴); W³ is C(R³); and W² is C(R²). In some embodiments of Formula (III), or sub-formulae thereof, W⁴ is C(O), S(O), S(O)₂, S(O)(NR⁴), S(O)(R⁴), or P(O)(R⁴); W³ is N; and W² is N. In some embodiments of Formula (III), or sub-formulae thereof, W¹ is C(O), S(O), S(O)₂, S(O)(NR¹), S(O)(R¹), or P(O)(R¹); W³ is N; and W² is C(R²). In some embodiments of Formula (III), or sub-formulae thereof, W¹ is C(O), S(O), S(O)₂, S(O)(NR¹), S(O)(R¹), or P(O)(R¹); W² is N; and W³ is C(R³). In some embodiments of Formula (III), or sub-formulae thereof, W¹ is C(O), S(O), S(O)₂, S(O)(NR¹), S(O)(R¹), or P(O)(R¹); W³ is C(R³); and W² is C(R²). In some embodiments of Formula (III), or sub-formulae thereof, W¹ is C(O), S(O), S(O)₂, S(O)(NR¹), S(O)(R¹), or P(O)(R¹); W³ is N; and W² is N.

In some embodiments (e.g., including Formulae (I), (II), (III), (IV); or sub-formulae thereof), R¹² is independently hydrogen. In some embodiments (e.g., including Formulae (I), (II), (III), (IV); or sub-formulae thereof), R¹² is independently C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, or C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments (e.g., including Formulae (I), (II), (III), (IV); or sub-formulae thereof), R¹² is independently C₁₋₆alkyl optionally substituted with one, two, or three R^{20k}. In some embodiments (e.g., including Formulae (I), (II), (III), (IV); or sub-formulae thereof), R¹² is independently C₁₋₆alkyl. In some embodiments (e.g., including Formulae (I), (II), (III), (IV); or sub-formulae thereof), R¹² is independently C₁₋₄alkyl. In some embodiments (e.g., including Formulae (I), (II), (III), (IV); or sub-formulae thereof), R¹² is independently C₁₋₃alkyl. In some embodiments (e.g., including Formulae (I), (II), (III), (IV); or sub-formulae thereof), R¹² is independently C₁₋₂alkyl. In some embodiments (e.g., including Formulae (I), (II), (III), (IV); or sub-formulae thereof), R¹² is methyl.

In some embodiments (e.g., including Formulae (I), (II), (III), (IV); or sub-formulae thereof), R^{12a} is independently hydrogen. In some embodiments (e.g., including Formulae (I), (II), (III), (IV); or sub-formulae thereof), R^{12a} is independently C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments (e.g., including Formulae (I), (II), (III), (IV); or sub-formulae thereof), R^{12a} is independently C₁₋₆alkyl optionally substituted with one, two, or three R^{20k}. In some embodiments (e.g., including Formulae (I), (II), (III), (IV); or sub-formulae thereof), R^{12a} is independently C₁₋₆alkyl. In some embodiments (e.g., including Formulae (I), (II), (III), (IV); or sub-formulae thereof), R^{12a} is independently C₁₋₄alkyl. In some embodiments (e.g., including Formulae (I), (II), (III), (IV); or sub-formulae thereof), R¹² is independently C₁₋₃alkyl. In some embodiments (e.g., including Formulae (I), (II), (III), (IV); or sub-formulae thereof), R^{1a} is independently C₁₋₂alkyl. In some embodiments (e.g., including Formulae (I), (II), (III), (IV); or sub-formulae thereof), R^{12a} is methyl.

In some embodiments (e.g., including Formulae (I), (II), (III), (IV); or sub-formulae thereof), R¹² is independently hydrogen. In some embodiments (e.g., including Formulae (I), (II), (III), (IV); or sub-formulae thereof), R¹² is independently C₁₋₆alkyl. In some embodiments (e.g., including Formulae (I), (II), (III), (IV); or sub-formulae thereof), R¹² is independently C₂₋₆alkenyl. In some embodiments (e.g., including Formulae (I), (II), (III), (IV); or sub-formulae thereof), R¹² is independently C₂₋₆alkynyl. In some embodiments (e.g., including Formulae (I), (II), (III), (IV); or sub-formulae thereof), R¹² is independently C₃₋₆cycloalkyl. In some embodiments (e.g., including Formulae (I), (II), (III), (IV); or sub-formulae thereof), R¹² is independently —CH₂—C₃₋₆cycloalkyl. In some embodiments (e.g., including Formulae (I), (II), (III), (IV); or sub-formulae thereof), R¹² is independently C₂₋₉heterocycloalkyl.

In some embodiments (e.g., including Formulae (I), (II), (III), (IV); or sub-formulae thereof), R¹² is independently —CH₂—C₂₋₉heterocycloalkyl. In some embodiments (e.g., including Formulae (I), (II), (III), (IV); or sub-formulae thereof), R¹² is independently C₆₋₁₀aryl. In some embodiments (e.g., including Formulae (I), (II), (III), (IV); or sub-formulae thereof), R¹² is independently —CH₂—C₆₋₁₀aryl. In some embodiments (e.g., including Formulae (I), (II), (III), (IV); or sub-formulae thereof), R¹² is independently C₁₋₉ heteroaryl. In some embodiments (e.g., including Formulae (I), (II), (III), (IV); or sub-formulae thereof), R¹² is independently C₁₋₆alkyl substituted with one, two, or three R^{20k}. In some embodiments (e.g., including Formulae (I), (II), (III), (IV); or sub-formulae thereof), R¹² is independently C₂₋₆alkenyl substituted with one, two, or three R^{20k}. In some embodiments (e.g., including Formulae (I), (II), (III), (IV); or sub-formulae thereof), R¹² is independently C₂₋₆alkynyl substituted with one, two, or three R^{20k}. In some embodiments (e.g., including Formulae (I), (II), (III), (IV); or sub-formulae thereof), R¹² is independently C₃₋₆cycloalkyl substituted with one, two, or three R^{20k}. In some embodiments (e.g., including Formulae (I), (II), (III), (IV); or sub-formulae thereof), R¹² is independently —CH₂—C₃₋₆cycloalkyl substituted with one, two, or three R^{20k}. In some embodiments (e.g., including Formulae (I), (II), (III), (IV); or sub-formulae thereof), R¹² is independently C₂₋₉heterocycloalkyl substituted with one, two, or three R^{20k}. In some embodiments (e.g., including Formulae (I), (II), (III), (IV); or sub-formulae thereof), R¹² is independently —CH₂—C₂₋₉heterocycloalkyl substituted with one, two, or three R^{20k}. In some embodiments (e.g., including Formulae (I), (II), (III), (IV); or sub-formulae thereof), R¹² is independently C₆₋₁₀aryl substituted with one, two, or three R^{20k}. In some embodiments (e.g., including Formulae (I), (II), (III), (IV); or sub-formulae thereof), R¹² is independently —CH₂—C₆₋₁₀aryl substituted with one, two, or three R^{20k}. In some embodiments (e.g., including Formulae (I), (II), (III), (IV); or sub-formulae thereof), R¹² is independently C₁₋₉heteroaryl substituted with one, two, or three R^{20k}.

In some embodiments (e.g., including Formulae (I), (II), (III), (IV); or sub-formulae thereof), R^{12a} is independently hydrogen. In some embodiments (e.g., including Formulae (I), (II), (III), (IV); or sub-formulae thereof), R^{12a} is independently C₁₋₆alkyl. In some embodiments (e.g., including Formulae (I), (II), (III), (IV); or sub-formulae thereof), R^{1a} is independently C₂₋₆alkenyl. In some embodiments (e.g., including Formulae (I), (II), (III), (IV); or sub-formulae thereof), R^{1a} is independently C₂₋₆alkynyl. In some embodiments (e.g., including Formulae (I), (II), (III), (IV); or sub-formulae thereof), R^{1a} is independently C₃₋₆cycloalkyl. In some embodiments (e.g., including Formulae (I), (II), (III), (IV); or sub-formulae thereof), R^{12a} is independently —CH₂—C₃₋₆cycloalkyl. In some embodiments (e.g., including Formulae (I), (II), (III), (IV); or sub-formulae thereof), R^{12a} is independently —CH₂—C₂₋₉heterocycloalkyl. In some embodiments (e.g., including Formulae (I), (II), (III), (IV); or sub-formulae thereof), R^{12a} is independently C₆₋₁₀aryl. In some embodiments (e.g., including Formulae (I), (II), (III), (IV); or sub-formulae thereof), R^{12a} is independently —CH₂—C₆₋₁₀aryl. In some embodiments (e.g., including Formulae (I), (II), (III), (IV); or sub-formulae thereof), R^{12a} is independently C₁₋₉ heteroaryl. In some embodiments (e.g., including Formulae (I), (II), (III), (IV); or sub-formulae thereof), R^{1a} is independently C₁₋₆alkyl substituted with one, two, or three R^{20k}. In some embodiments (e.g., including Formulae (I), (II), (III), (IV); or sub-formulae thereof), R^{12a} is independently C₂₋₆alkenyl substituted with one, two, or three R^{20k}. In some embodiments (e.g., including Formulae (I), (II), (III), (IV); or sub-formulae thereof), R^{1a} is independently C₂₋₆alkynyl substituted with one, two, or three R^{20k}. In some embodiments (e.g., including Formulae (I), (II), (III), (IV); or sub-formulae thereof), R^{1a} is independently C₃₋₆cycloalkyl substituted with one, two, or three R^{20k}. In some embodiments (e.g., including Formulae (I), (II), (III), (IV); or sub-formulae thereof), R^{12a} is independently —CH₂—C₃₋₆cycloalkyl substituted with one, two, or three R^{20k}. In some embodiments (e.g., including Formulae (I), (II), (III), (IV); or sub-formulae thereof), R^{12a} is independently —CH₂—C₂₋₉heterocycloalkyl substituted with one, two, or three R^{20k}. In some embodiments (e.g., including Formulae (I), (II), (III), (IV); or sub-formulae thereof), R¹² is independently C₆₋₁₀aryl substituted with one, two, or three R^{20k}. In some embodiments (e.g., including Formulae (I), (II), (III), (IV); or sub-formulae thereof), R^{1a} is independently —CH₂—C₆₋₁₀aryl substituted with one, two, or three R^{20k}. In some embodiments (e.g., including Formulae (I), (II), (III), (IV); or sub-formulae thereof), R^{12a} is independently C₁₋₉heteroaryl substituted with one, two, or three R^{20k}.

In some embodiments (e.g., including Formulae (I), (II), (III), (IV); or sub-formulae thereof), R¹³ is independently hydrogen. In some embodiments (e.g., including Formulae (I), (II), (III), (IV); or sub-formulae thereof), R¹³ is independently C₁₋₆alkyl. In some embodiments (e.g., including Formulae (I), (II), (III), (IV); or sub-formulae thereof), R¹³ is independently C₁₋₆haloalkyl.

In some embodiments (e.g., including Formulae (I), (II), (III), (IV); or sub-formulae thereof), R¹² and R¹³, together with the nitrogen to which they are attached, form a C₂₋₉heterocycloalkyl ring. In some embodiments (e.g., including Formulae (I), (II), (III), (IV); or sub-formulae thereof), R¹² and R¹³, together with the nitrogen to which they are attached, form a C₂₋₉heterocycloalkyl ring substituted with one, two, or three R^{20k}.

In some embodiments (e.g., including Formulae (I), (II), (III), (IV); or sub-formulae thereof), R¹⁴ is independently hydrogen. In some embodiments (e.g., including Formulae (I), (II), (III), (IV); or sub-formulae thereof), R¹⁴ is independently C₁₋₆alkyl. In some embodiments (e.g., including Formulae (I), (II), (III), (IV); or sub-formulae thereof), R¹⁴ is independently C₁₋₆haloalkyl.

In some embodiments (e.g., including Formulae (I), (II), (III), (IV); or sub-formulae thereof), R¹⁵ is independently C₁₋₆alkyl. In some embodiments (e.g., including Formulae (I), (II), (III), (IV); or sub-formulae thereof), R¹⁵ is independently C₂₋₆alkenyl. In some embodiments (e.g., including Formulae (I), (II), (III), (IV); or sub-formulae thereof), R¹⁵ is independently C₂₋₆alkynyl. In some embodiments (e.g., including Formulae (I), (II), (III), (IV); or sub-formulae thereof), R¹⁵ is independently C₃₋₆cycloalkyl. In some embodiments (e.g., including Formulae (I), (II), (III), (IV); or sub-formulae thereof), R¹⁵ is independently C₁₋₆heteroalkyl. In some embodiments (e.g., including Formulae (I), (II), (III), (IV); or sub-formulae thereof), R¹⁵ is independently C₂₋₉heterocycloalkyl. In some embodiments (e.g., including Formulae (I), (II), (III), (IV); or sub-formulae thereof), R¹⁵ is independently C₆₋₁₀aryl. In some embodiments (e.g., including Formulae (I), (II), (III), (IV); or sub-formulae thereof), R¹⁵ is independently C₁₋₉heteroaryl. In some embodiments (e.g., including Formulae (I), (II), (III), (IV); or sub-formulae thereof), R¹⁵ is independently C₁₋₆alkyl substituted with one, two, or three R^{20k}. In some embodiments (e.g., including Formulae (I), (II), (III), (IV); or sub-formulae thereof), R¹⁵ is independently C₂₋₆alkenyl substituted with one, two, or three R^{20k}. In some embodiments (e.g., including Formulae (I), (II), (III), (IV); or sub-formulae thereof), R¹⁵ is independently C₂₋₆alkynyl substituted with one, two, or three R^{20k}. In some embodiments (e.g., including Formulae (I), (II), (III), (IV); or sub-formulae thereof), R¹⁵ is independently C₃₋₆cycloalkyl substituted with one, two, or three R^{20k}. In some embodiments (e.g., including Formulae (I), (II), (III), (IV); or sub-formulae thereof), R¹⁵ is independently C₁₋₆ heteroalkyl substituted with one, two, or three R^{20k}. In some embodiments (e.g., including Formulae (I), (II), (III), (IV); or sub-formulae thereof), R¹⁵ is independently C₂₋₉heterocycloalkyl substituted with one, two, or three R^{20k}. In some embodiments (e.g., including Formulae (I), (II), (III), (IV); or sub-formulae thereof), R¹⁵ is independently C₆₋₁₀aryl substituted with one, two, or three R^{20k}. In some embodiments (e.g., including Formulae (I), (II), (III), (IV); or sub-formulae thereof), R¹⁵ is independently C₁₋₉heteroaryl substituted with one, two, or three R^{20k}. In some embodiments (e.g., including Formulae (I), (II), (III), (IV); or sub-formulae thereof), R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20k}. In some embodiments (e.g., including Formulae (I), (II), (III), (IV); or sub-formulae thereof), R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In some embodiments (e.g., including Formulae (I), (II), (III), (IV); or sub-formulae thereof), R¹⁵ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20k}.

In some embodiments (e.g., including Formulae (I), (II), (III), (IV); or sub-formulae thereof), R¹⁶ is independently —C₁₋₆ alkylene-OP(O)(OR^{16a})(OR^{16b}). In some embodiments (e.g., including Formulae (I), (II), (III), (IV); or sub-formulae thereof), R¹⁶ is independently —C₁₋₆alkylene-OP(O)(OR^{16a})(OR^{16b}), wherein the C₁₋₆alkylene is substituted with one, two, or three R^{20l}. In some embodiments (e.g., including Formulae (I), (II), (III), (IV); or sub-formulae thereof), R¹⁶ is independently —P(O)(OR^{16a})(OR^{16b}).

In some embodiments (e.g., including Formulae (I), (II), (III), (IV); or sub-formulae thereof), R¹⁶ is independently —C₁alkylene-OP(O)(OR¹⁶)(OR^{16b}). In some embodiments (e.g., including Formulae (I), (II), (III), (IV); or sub-formulae thereof), R¹⁶ is independently —C₁alkylene-OP(O)(OR^{16a})(OR^{61b}), wherein the C₁alkylene is substituted with one or two R^{20l}. In some embodiments (e.g., including Formulae (I), (II), (III), (IV); or sub-formulae thereof), R¹⁶ is independently —C₂alkylene-OP(O)(OR^{16a})(OR^{16b}). In some embodiments (e.g., including Formulae (I), (II), (III), (IV); or sub-formulae thereof), R¹⁶ is independently −C₂alkylene-OP(O)(OR^{16a})(OR^{16b}), wherein the C₂alkylene is substituted with one, two, or three R^{20l}. In some embodiments (e.g., including Formulae (I), (II), (III), (IV); or sub-formulae thereof), R¹⁶ is independently-C₃alkylene-OP(O)(OR^{16a})(OR^{16b}). In some embodiments (e.g., including Formulae (I), (II), (III), (IV); or sub-formulae thereof), R¹⁶ is independently-C₃alkylene-OP(O)(OR¹⁶)(OR^{16b}), wherein the C₃alkylene is substituted with one, two, or three R^{20l}. In some embodiments (e.g., including Formulae (I), (II), (III), (IV); or sub-formulae thereof), R¹⁶ is independently-C₄alkylene-OP(O)(OR^{16a})(OR^{16b}). In some embodiments (e.g., including Formulae (I), (II), (III), (IV); or sub-formulae thereof), R¹⁶ is independently-C₄alkylene-OP(O)(OR¹⁶)(OR^{16b}), wherein the C₄alkylene is substituted with one, two, or three R^{20l}. In some embodiments (e.g., including Formulae (I), (II), (III), (IV); or sub-formulae thereof), R¹⁶ is independently-C₅alkylene-OP(O)(OR^{16a})(OR^{16b}). In some embodiments (e.g., including Formulae (I), (II), (III), (IV); or sub-formulae thereof), R¹⁶ is independently-C₅alkylene-OP(O)(OR^{16a})(OR^{16b}), wherein the C₅alkylene is substituted with one, two, or three R^{20l}. In some embodiments (e.g., including Formulae (I), (II), (III), (IV); or sub-formulae thereof), R¹⁶ is independently-C₆alkylene-OP(O)(OR¹⁶)(OR^{16b}). In some embodiments (e.g., including Formulae (I), (II), (III), (IV); or sub-formulae thereof), R¹⁶ is independently-C₆alkylene-OP(O)(OR^{16a})(OR⁶I), wherein the C₆alkylene is substituted with one, two, or three R^{20l}.

In some embodiments (e.g., including Formulae (I), (II), (III), (IV); or sub-formulae thereof), R^{16a} is independently hydrogen. In some embodiments (e.g., including Formulae (I), (II), (III), (IV); or sub-formulae thereof), R^{16a} is independently C₁₋₆alkyl. In some embodiments (e.g., including Formulae (I), (II), (III), (IV); or sub-formulae thereof), R¹⁶ is independently C₂₋₆alkenyl. In some embodiments (e.g., including Formulae (I), (II), (III), (IV); or sub-formulae thereof), R¹⁶ is independently C₂₋₆alkynyl. In some embodiments (e.g., including Formulae (I), (II), (III), (IV); or sub-formulae thereof), R¹⁶ is independently C₃₋₆cycloalkyl. In some embodiments (e.g., including Formulae (I), (II), (III), (IV); or sub-formulae thereof), R¹⁶ is independently —CH₂—C₃₋₆cycloalkyl. In some embodiments (e.g., including Formulae (I), (II), (III), (IV); or sub-formulae thereof), R¹⁶ is independently —CH₂—C₂₋₉heterocycloalkyl. In some embodiments (e.g., including Formulae (I), (II), (III), (IV); or sub-formulae thereof), R¹⁶ is independently C₆₋₁₀aryl. In some embodiments (e.g., including Formulae (I), (II), (III), (IV); or sub-formulae thereof), R¹⁶ is independently —CH₂—C₆₋₁₀aryl. In some embodiments (e.g., including Formulae (I), (II), (III), (IV); or sub-formulae thereof), R¹⁶ is independently C₁₋₉ heteroaryl. In some embodiments (e.g., including Formulae (I), (II), (III), (IV); or sub-formulae thereof), R¹⁶ is independently C₁₋₆alkyl substituted with one, two, or three R². In some embodiments (e.g., including Formulae (I), (II), (III), (IV); or sub-formulae thereof), R^{16a} is independently C₂₋₆alkenyl substituted with one, two, or three R^{20m}. In some embodiments (e.g., including Formulae (I), (II), (III), (IV); or sub-formulae thereof), R¹⁶ is independently C₂₋₆alkynyl substituted with one, two, or three R^{20m}. In some embodiments (e.g., including Formulae (I), (II), (III), (IV); or sub-formulae thereof), R¹⁶ is independently C₃₋₆cycloalkyl substituted with one, two, or three R^{20m}. In some embodiments (e.g., including Formulae (I), (II), (III), (IV); or sub-formulae thereof), R^{16a} is independently —CH₂—C₃₋₆cycloalkyl substituted with one, two, or three R^{20m}. In some embodiments (e.g., including Formulae (I), (II), (III), (IV); or sub-formulae thereof), R^{16a} is independently —CH₂—C₂₋₉heterocycloalkyl substituted with one, two, or three R^{20m}. In some embodiments (e.g., including Formulae (I), (II), (III), (IV); or sub-formulae thereof), R¹⁶ is independently C₆₋₁₀aryl substituted with one, two, or three R². In some embodiments (e.g., including Formulae (I), (II), (III), (IV); or sub-formulae thereof), R^{16a} is independently —CH₂—C₆₋₁₀aryl substituted with one, two, or three R². In some embodiments (e.g., including Formulae (I), (II), (III), (IV); or sub-formulae thereof), R^{16a} is independently C₁₋₉heteroaryl substituted with one, two, or three R^{20m}.

In some embodiments (e.g., including Formulae (I), (II), (III), (IV); or sub-formulae thereof), R^{16b} is independently hydrogen. In some embodiments (e.g., including Formulae (I), (II), (III), (IV); or sub-formulae thereof), R^{16b} is independently C₁₋₆alkyl. In some embodiments (e.g., including Formulae (I), (II), (III), (IV); or sub-formulae thereof), R^{16b} is independently C₂₋₆alkenyl. In some embodiments (e.g., including Formulae (I), (II), (III), (IV); or sub-formulae thereof), R^{16b} is independently C₂₋₆alkynyl. In some embodiments (e.g., including Formulae (I), (II), (III), (IV); or sub-formulae thereof), R^{16b} is independently C₃₋₆cycloalkyl. In some embodiments (e.g., including Formulae (I), (II), (III), (IV); or sub-formulae thereof), R^{16b} is independently —CH₂—C₃₋₆cycloalkyl. In some embodiments (e.g., including Formulae (I), (II), (III), (IV); or sub-formulae thereof), R^{16b} is independently —CH₂—C₂₋₉heterocycloalkyl. In some embodiments (e.g., including Formulae (I), (II), (III), (IV); or sub-formulae thereof), R^{16b} is independently C₆₋₁₀aryl. In some embodiments (e.g., including Formulae (I), (II), (III), (IV); or sub-formulae thereof), R^{16b} is independently —CH₂—C₆₋₁₀aryl. In some embodiments (e.g., including Formulae (I), (II), (III), (IV); or sub-formulae thereof), R^{16b} is independently C₁₋₉ heteroaryl. In some embodiments (e.g., including Formulae (I), (II), (III), (IV); or sub-formulae thereof), R^{16b} is independently C₁₋₆ alkyl substituted with one, two, or three R^{20m}. In some embodiments (e.g., including Formulae (I), (II), (III), (IV); or sub-formulae thereof), R^{16b} is independently C₂₋₆alkenyl substituted with one, two, or three R^{20m}. In some embodiments (e.g., including Formulae (I), (II), (III), (IV); or sub-formulae thereof), R^{16b} is independently C₂₋₆alkynyl substituted with one, two, or three R^{20m}. In some embodiments (e.g., including Formulae (I), (II), (III), (IV); or sub-formulae thereof), R^{16b} is independently C₃₋₆cycloalkyl substituted with one, two, or three R^{20m}. In some embodiments (e.g., including Formulae (I), (II), (III), (IV); or sub-formulae thereof), R^{16b} is independently —CH₂—C₃₋₆cycloalkyl substituted with one, two, or three R^{20m}. In some embodiments (e.g., including Formulae (I), (II), (III), (IV); or sub-formulae thereof), R^{16b} is independently —CH₂—C₂₋₉heterocycloalkyl substituted with one, two, or three R^{20m}. In some embodiments (e.g., including Formulae (I), (II), (III), (IV); or sub-formulae thereof), R^{16b} is independently C₆₋₁₀aryl substituted with one, two, or three R^{20m}. In some embodiments (e.g., including Formulae (I), (II), (III), (IV); or sub-formulae thereof), R^{16b} is independently —CH₂—C₆₋₁₀aryl substituted with one, two, or three R². In some embodiments (e.g., including Formulae (I), (II), (III), (IV); or sub-formulae thereof), R^{16b} is independently C₁₋₉heteroaryl substituted with one, two, or three R^{20m}.

In some embodiments (e.g., including Formulae (XI), (XII), (XIII), and/or (XIV); or sub-formulae thereof), X is N. In some embodiments (e.g., including Formulae (XI), (XII), (XIII), and/or (XIV); or sub-formulae thereof), X is C(R⁵). In some embodiments (e.g., including Formulae (XI), (XII), (XIII), and/or (XIV); or sub-formulae thereof), X is CH. In some embodiments (e.g., including Formulae (XI), (XII), (XIII), and/or (XIV); or sub-formulae thereof), X is C(halogen). In some embodiments (e.g., including Formulae (XI), (XII), (XIII), and/or (XIV); or sub-formulae thereof), X is C(F).

In some embodiments (e.g., including Formulae (XI), (XII), (XIII), and/or (XIV); or sub-formulae thereof), R⁵ is hydrogen. In some embodiments (e.g., including Formulae (XI), (XII), (XIII), and/or (XIV); or sub-formulae thereof), R⁵ is halogen. In some embodiments (e.g., including Formulae (XI), (XII), (XIII), and/or (XIV); or sub-formulae thereof), R⁵ is —F. In some embodiments (e.g., including Formulae (XI), (XII), (XIII), and/or (XIV); or sub-formulae thereof), R⁵ is —Cl. In some embodiments (e.g., including Formulae (XI), (XII), (XIII), and/or (XIV); or sub-formulae thereof), R⁵ is —Br. In some embodiments (e.g., including Formulae (XI), (XII), (XIII), and/or (XIV); or sub-formulae thereof), R⁵ is —I. In some embodiments (e.g., including Formulae (XI), (XII), (XIII), and/or (XIV); or sub-formulae thereof), R⁵ is —CN. In some embodiments (e.g., including Formulae (XI), (XII), (XIII), and/or (XIV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e}. In some embodiments (e.g., including Formulae (XI), (XII), (XIII), and/or (XIV); or sub-formulae thereof), R⁵ is C₁₋₆alkyl.

In some embodiments (e.g., including Formulae (XI), (XII), (XIII), and/or (XIV); or sub-formulae thereof), R⁵ is C₁₋₄alkyl. In some embodiments (e.g., including Formulae (XI), (XII), (XIII), and/or (XIV); or sub-formulae thereof), R⁵ is C₁₋₃alkyl. In some embodiments (e.g., including Formulae (XI), (XII), (XIII), and/or (XIV); or sub-formulae thereof), R⁵ is C₁₋₂alkyl. In some embodiments (e.g., including Formulae (XI), (XII), (XIII), and/or (XIV); or sub-formulae thereof), R⁵ is methyl.

In some embodiments (e.g., including Formulae (XI), (XII), (XIII), and/or (XIV); or sub-formulae thereof), R⁵ is hydrogen. In some embodiments (e.g., including Formulae (XI), (XII), (XIII), and/or (XIV); or sub-formulae thereof), R⁵ is halogen. In some embodiments (e.g., including Formulae (XI), (XII), (XIII), and/or (XIV); or sub-formulae thereof), R⁵ is —CN. In some embodiments (e.g., including Formulae (XI), (XII), (XIII), and/or (XIV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R⁵ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In some embodiments (e.g., including Formulae (XI), (XII), (XIII), and/or (XIV); or sub-formulae thereof), R⁵ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20e}.

In some embodiments (e.g., including Formulae (XI), (XII), (XIII), and/or (XIV); or sub-formulae thereof), R⁵ is selected from hydrogen, halogen, and C₁₋₆alkyl optionally substituted with one, two, or three R^{20e}. In some embodiments (e.g., including Formulae (XI), (XII), (XIII), and/or (XIV); or sub-formulae thereof), R⁵ is selected from hydrogen and halogen.

In some embodiments (e.g., including Formulae (XI), (XII), (XIII), and/or (XIV); or sub-formulae thereof), Y is N. In some embodiments (e.g., including Formulae (XI), (XII), (XIII), and/or (XIV); or sub-formulae thereof), Y is C(R⁶). In some embodiments (e.g., including Formulae (XI), (XII), (XIII), and/or (XIV); or sub-formulae thereof), Y is CH. In some embodiments (e.g., including Formulae (XI), (XII), (XIII), and/or (XIV); or sub-formulae thereof), Y is C(R⁶). In some embodiments (e.g., including Formulae (XI), (XII), (XIII), and/or (XIV); or sub-formulae thereof), Y is C(OH).

In some embodiments (e.g., including Formulae (XI), (XII), (XIII), and/or (XIV); or sub-formulae thereof), R⁶ is —OR¹⁶. In some embodiments (e.g., including Formulae (XI), (XII), (XIII), and/or (XIV); or sub-formulae thereof), R⁶ is —N(R¹²)(R¹⁶). In some embodiments (e.g., including Formulae (XI), (XII), (XIII), and/or (XIV); or sub-formulae thereof), R⁶ is —OR¹². In some embodiments (e.g., including Formulae (XI), (XII), (XIII), and/or (XIV); or sub-formulae thereof), R⁶ is —OR^{12a}. In some embodiments (e.g., including Formulae (XI), (XII), (XIII), and/or (XIV); or sub-formulae thereof), R⁶ is —SR¹². In some embodiments (e.g., including Formulae (XI), (XII), (XIII), and/or (XIV); or sub-formulae thereof), R⁶ is —N(R¹²)(R¹³). In some embodiments (e.g., including Formulae (XI), (XII), (XIII), and/or (XIV); or sub-formulae thereof), R⁶ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl optionally substituted with one, two, or three R^{20f}. In some embodiments (e.g., including Formulae (XI), (XII), (XIII), and/or (XIV); or sub-formulae thereof), R⁶ is —OH. In some embodiments (e.g., including Formulae (XI), (XII), (XIII), and/or (XIV); or sub-formulae thereof), R⁶ is C₁₋₆alkyl. In some embodiments (e.g., including Formulae (XI), (XII), (XIII), and/or (XIV); or sub-formulae thereof), R⁶ is C₁₋₄alkyl. In some embodiments (e.g., including Formulae (XI), (XII), (XIII), and/or (XIV); or sub-formulae thereof), R⁶ is C₁₋₃alkyl. In some embodiments (e.g., including Formulae (XI), (XII), (XIII), and/or (XIV); or sub-formulae thereof), R⁶ is C₁₋₂alkyl. In some embodiments (e.g., including Formulae (XI), (XII), (XIII), and/or (XIV); or sub-formulae thereof), R⁶ is methyl.

In some embodiments (e.g., including Formulae (XI), (XII), (XIII), and/or (XIV); or sub-formulae thereof), R⁶ is hydrogen. In some embodiments (e.g., including Formulae (XI), (XII), (XIII), and/or (XIV); or sub-formulae thereof), R⁶ is halogen. In some embodiments (e.g., including Formulae (XI), (XII), (XIII), and/or (XIV); or sub-formulae thereof), R⁶ is —CN. In some embodiments (e.g., including Formulae (XI), (XII), (XIII), and/or (XIV); or sub-formulae thereof), R⁶ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R⁶ is —OR¹⁶. In some embodiments (e.g., including Formulae (XI), (XII), (XIII), and/or (XIV); or sub-formulae thereof), R⁶ is —N(R¹²)(R¹⁶). In some embodiments (e.g., including Formulae (XI), (XII), (XIII), and/or (XIV); or sub-formulae thereof), R⁶ is —OR¹². In some embodiments (e.g., including Formulae (XI), (XII), (XIII), and/or (XIV); or sub-formulae thereof), R⁶ is —OR^{12a}. In some embodiments (e.g., including Formulae (XI), (XII), (XIII), and/or (XIV); or sub-formulae thereof), R⁶ is —SR¹². In some embodiments (e.g., including Formulae (XI), (XII), (XIII), and/or (XIV); or sub-formulae thereof), R⁶ is —N(R¹²)(R¹³). In some embodiments (e.g., including Formulae (XI), (XII), (XIII), and/or (XIV); or sub-formulae thereof), R⁶ is —C(O)OR¹². In some embodiments (e.g., including Formulae (XI), (XII), (XIII), and/or (XIV); or sub-formulae thereof), R⁶ is —OC(O)N(R¹²)(R¹³). In some embodiments (e.g., including Formulae (XI), (XII), (XIII), and/or (XIV); or sub-formulae thereof), R⁶ is —N(R¹⁴)C(O)N(R¹²)(R¹³). In some embodiments (e.g., including Formulae (XI), (XII), (XIII), and/or (XIV); or sub-formulae thereof), R⁶ is —N(R¹⁴)C(O)OR¹². In some embodiments (e.g., including Formulae (XI), (XII), (XIII), and/or (XIV); or sub-formulae thereof), R⁶ is —N(R¹⁴)S(O)₂R¹⁵. In some embodiments (e.g., including Formulae (XI), (XII), (XIII), and/or (XIV); or sub-formulae thereof), R⁶ is —C(O)R¹⁵. In some embodiments (e.g., including Formulae (XI), (XII), (XIII), and/or (XIV); or sub-formulae thereof), R⁶ is —S(O)R¹⁵. In some embodiments (e.g., including Formulae (XI), (XII), (XIII), and/or (XIV); or sub-formulae thereof), R⁶ is —OC(O)R¹⁵. In some embodiments (e.g., including Formulae (XI), (XII), (XIII), and/or (XIV); or sub-formulae thereof), R⁶ is —C(O)N(R¹²)(R¹³). In some embodiments (e.g., including Formulae (XI), (XII), (XIII), and/or (XIV); or sub-formulae thereof), R⁶ is —C(O)C(O)N(R¹²)(R¹³). In some embodiments (e.g., including Formulae (XI), (XII), (XIII), and/or (XIV); or sub-formulae thereof), R⁶ is —N(R¹⁴)C(O)R¹⁵. In some embodiments (e.g., including Formulae (XI), (XII), (XIII), and/or (XIV); or sub-formulae thereof), R⁶ is —S(O)₂R¹⁵. In some embodiments (e.g., including Formulae (XI), (XII), (XIII), and/or (XIV); or sub-formulae thereof), R⁶ is —S(O)₂N(R¹²)(R¹³). In some embodiments (e.g., including Formulae (XI), (XII), (XIII), and/or (XIV); or sub-formulae thereof), R⁶ is —S(═O)(═NH)N(R¹²)(R¹³). In some embodiments (e.g., including Formulae (XI), (XII), (XIII), and/or (XIV); or sub-formulae thereof), R⁶ is —CH₂C(O)N(R¹²)(R¹³). In some embodiments (e.g., including Formulae (XI), (XII), (XIII), and/or (XIV); or sub-formulae thereof), R⁶ is —CH₂N(R¹⁴)C(O)R¹⁵. In some embodiments (e.g., including Formulae (XI), (XII), (XIII), and/or (XIV); or sub-formulae thereof), R⁶ is —CH₂S(O)₂R¹⁵. In some embodiments (e.g., including Formulae (XI), (XII), (XIII), and/or (XIV); or sub-formulae thereof), R⁶ is —CH₂S(O)₂N(R¹²)(R¹³). In some embodiments (e.g., including Formulae (XI), (XII), (XIII), and/or (XIV); or sub-formulae thereof), R⁶ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20f}.

In some embodiments (e.g., including Formulae (XI), (XII), (XIII), and/or (XIV); or sub-formulae thereof), R⁶ is selected from hydrogen, halogen, —OR¹a, and C₁₋₆alkyl optionally substituted with one, two, or three R^{20f}.

In some embodiments of Formulae (I), or sub-formulae thereof, J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of Formulae (I), or sub-formulae thereof, J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of Formulae (I), or sub-formulae thereof, J³ is CH₂ and R⁶ is —OR¹⁶. In some embodiments of Formulae (I), or sub-formulae thereof, J³ is CH₂ and R⁶ is —N(R¹²)(R¹⁶).

In some embodiments (e.g., including Formulae (XI), (XII), (XIII), and/or (XIV); or sub-formulae thereof), Z is N. In some embodiments (e.g., including Formulae (XI), (XII), (XIII), and/or (XIV); or sub-formulae thereof), Z is C(R⁷). In some embodiments (e.g., including Formulae (XI), (XII), (XIII), and/or (XIV); or sub-formulae thereof), Z is CH. In some embodiments (e.g., including Formulae (XI), (XII), (XIII), and/or (XIV); or sub-formulae thereof), Z is C(R⁷).

In some embodiments (e.g., including Formulae (XI), (XII), (XIII), and/or (XIV); or sub-formulae thereof), R⁷ is hydrogen. In some embodiments (e.g., including Formulae (XI), (XII), (XIII), and/or (XIV); or sub-formulae thereof), R⁷ is halogen. In some embodiments (e.g., including Formulae (XI), (XII), (XIII), and/or (XIV); or sub-formulae thereof), R⁷ is —CN. In some embodiments (e.g., including Formulae (XI), (XII), (XIII), and/or (XIV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g}. In some embodiments (e.g., including Formulae (XI), (XII), (XIII), and/or (XIV); or sub-formulae thereof), R⁷ is C₁₋₆alkyl. In some embodiments (e.g., including Formulae (XI), (XII), (XIII), and/or (XIV); or sub-formulae thereof), R⁷ is C₁₋₄alkyl. In some embodiments (e.g., including Formulae (XI), (XII), (XIII), and/or (XIV); or sub-formulae thereof), R⁷ is C₁₋₂alkyl. In some embodiments (e.g., including Formulae (XI), (XII), (XIII), and/or (XIV); or sub-formulae thereof), R⁷ is methyl.

In some embodiments (e.g., including Formulae (XI), (XII), (XIII), and/or (XIV); or sub-formulae thereof), R⁷ is hydrogen. In some embodiments (e.g., including Formulae (XI), (XII), (XIII), and/or (XIV); or sub-formulae thereof), R⁷ is halogen. In some embodiments (e.g., including Formulae (XI), (XII), (XIII), and/or (XIV); or sub-formulae thereof), R⁷ is —CN. In some embodiments (e.g., including Formulae (XI), (XII), (XIII), and/or (XIV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R⁷ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉ heteroaryl substituted with one, two, or three R^{20g}. In some embodiments (e.g., including Formulae (XI), (XII), (XIII), and/or (XIV); or sub-formulae thereof), R⁷ is selected from hydrogen, halogen, and C₁₋₆alkyl optionally substituted with one, two, or three R^{20g}.

In some embodiments (e.g., including Formulae (XI), (XII), (XIII), and/or (XIV); or sub-formulae thereof), R⁸ is hydrogen. In some embodiments (e.g., including Formulae (XI), (XII), (XIII), and/or (XIV); or sub-formulae thereof), R⁸ is halogen. In some embodiments (e.g., including Formulae (XI), (XII), (XIII), and/or (XIV); or sub-formulae thereof), R¹ is —CN. In some embodiments (e.g., including Formulae (XI), (XII), (XIII), and/or (XIV); or sub-formulae thereof), R⁸ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R⁸ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉ heteroaryl substituted with one, two, or three R^{20h}.

In some embodiments (e.g., including Formulae (XI), (XII), (XIII), and/or (XIV); or sub-formulae thereof), R⁹ is hydrogen. In some embodiments (e.g., including Formulae (XI), (XII), (XIII), and/or (XIV); or sub-formulae thereof), R⁹ is halogen. In some embodiments (e.g., including Formulae (XI), (XII), (XIII), and/or (XIV); or sub-formulae thereof), R⁹ is —CN. In some embodiments (e.g., including Formulae (XI), (XII), (XIII), and/or (XIV); or sub-formulae thereof), R⁹ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R⁹ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R⁹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R²⁰ⁱ.

In some embodiments (e.g., including Formulae (XI), (XII), (XIII), and/or (XIV); or sub-formulae thereof), R^{9a} is hydrogen.

In some embodiments (e.g., including Formulae (XI), (XII), (XIII), and/or (XIV); or sub-formulae thereof), R^{9a} is halogen. In some embodiments (e.g., including Formulae (XI), (XII), (XIII), and/or (XIV); or sub-formulae thereof), R^{9a} is —CN. In some embodiments (e.g., including Formulae (XI), (XII), (XIII), and/or (XIV); or sub-formulae thereof), R^{9a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R^{9a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R^{9a} is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R²⁰ⁱ.

In some embodiments (e.g., including Formulae (XI), (XII), (XIII), and/or (XIV); or sub-formulae thereof), R⁹ and R^{9a} are combined to form a C₃₋₆cycloalkyl. In some embodiments (e.g., including Formulae (XI), (XII), (XIII), and/or (XIV); or sub-formulae thereof), R⁹ and R^{9a} are combined to form a C₂₋₉heterocycloalkyl. In some embodiments (e.g., including Formulae (XI), (XII), (XIII), and/or (XIV); or sub-formulae thereof), R⁹ and R^{9a} are combined to form a C₃₋₆cycloalkyl substituted with one, two, or three R²⁰ⁱ. In some embodiments (e.g., including Formulae (XI), (XII), (XIII), and/or (XIV); or sub-formulae thereof), R⁹ and R^{9a} are combined to form a C₂₋₉heterocycloalkyl substituted with one, two, or three R²⁰ⁱ.

In some embodiments (e.g., including Formulae (XI), (XII), (XIII), and/or (XIV); or sub-formulae thereof), R⁹ and R^{9a} are hydrogen.

In some embodiments (e.g., including Formulae (XI), (XII), (XIII), and/or (XIV); or sub-formulae thereof), R¹⁰ is hydrogen. In some embodiments (e.g., including Formulae (XI), (XII), (XIII), and/or (XIV); or sub-formulae thereof), R¹⁰ is halogen. In some embodiments (e.g., including Formulae (XI), (XII), (XIII), and/or (XIV); or sub-formulae thereof), R¹⁰ is —CN. In some embodiments (e.g., including Formulae (XI), (XII), (XIII), and/or (XIV); or sub-formulae thereof), R¹⁰ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R¹⁰ is —R¹⁶. In some embodiments (e.g., including Formulae (XI), (XII), (XIII), and/or (XIV); or sub-formulae thereof), R¹⁰ is —OR¹⁶. In some embodiments (e.g., including Formulae (XI), (XII), (XIII), and/or (XIV); or sub-formulae thereof), R¹⁰ is —N(R¹²)(R¹⁶). In some embodiments (e.g., including Formulae (XI), (XII), (XIII), and/or (XIV); or sub-formulae thereof), R¹⁰ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R¹⁰ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20j}.

In some embodiments (e.g., including Formulae (XI), (XII), (XIII), and/or (XIV); or sub-formulae thereof), R^{10a} is hydrogen. In some embodiments (e.g., including Formulae (XI), (XII), (XIII), and/or (XIV); or sub-formulae thereof), R^{10a} is halogen. In some embodiments (e.g., including Formulae (XI), (XII), (XIII), and/or (XIV); or sub-formulae thereof), R^{10a} is —CN. In some embodiments (e.g., including Formulae (XI), (XII), (XIII), and/or (XIV); or sub-formulae thereof), R^{10a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R^{10a} is —R¹⁶. In some embodiments (e.g., including Formulae (XI), (XII), (XIII), and/or (XIV); or sub-formulae thereof), R^{10a} is —OR¹⁶. In some embodiments (e.g., including Formulae (XI), (XII), (XIII), and/or (XIV); or sub-formulae thereof), R^{10a} is —N(R¹²)(R¹⁶). In some embodiments (e.g., including Formulae (XI), (XII), (XIII), and/or (XIV); or sub-formulae thereof), R^{10a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R^{10a} is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20j}.

In some embodiments (e.g., including Formulae (XI), (XII), (XIII), and/or (XIV); or sub-formulae thereof), R¹⁰ and R^{10a} are combined to form a C₃₋₆cycloalkyl. In some embodiments (e.g., including Formulae (XI), (XII), (XIII), and/or (XIV); or sub-formulae thereof), R¹⁰ and R^{10a} are combined to form a C₂₋₉heterocycloalkyl. In some embodiments (e.g., including Formulae (XI), (XII), (XIII), and/or (XIV); or sub-formulae thereof), R¹⁰ and R^{10a} are combined to form a C₃₋₆cycloalkyl substituted with one, two, or three R^{20j}. In some embodiments (e.g., including Formulae (XI), (XII), (XIII), and/or (XIV); or sub-formulae thereof), R¹⁰ and R^{10a} are combined to form a C₂₋₉heterocycloalkyl substituted with one, two, or three R^{20j}.

In some embodiments (e.g., including Formulae (XI), (XII), (XIII), and/or (XIV); or sub-formulae thereof), J¹ is N. In some embodiments (e.g., including Formulae (XI), (XII), (XIII), and/or (XIV); or sub-formulae thereof), J¹ is C. In some embodiments (e.g., including Formulae (XI), (XII), (XIII), and/or (XIV); or sub-formulae thereof), J¹ is C(R⁸).

In some embodiments (e.g., including Formulae (XI), (XII), (XIII), and/or (XIV); or sub-formulae thereof), J² is N. In some embodiments (e.g., including Formulae (XI), (XII), (XIII), and/or (XIV); or sub-formulae thereof), J² is N(R⁹). In some embodiments (e.g., including Formulae (XI), (XII), (XIII), and/or (XIV); or sub-formulae thereof), J² is C(R⁹). In some embodiments (e.g., including Formulae (XI), (XII), (XIII), and/or (XIV); or sub-formulae thereof), J² is C(H). In some embodiments (e.g., including Formulae (XI), (XII), (XIII), and/or (XIV); or sub-formulae thereof), J² is C(R⁹)(R^{9a}). In some embodiments (e.g., including Formulae (XI), (XII), (XIII), and/or (XIV); or sub-formulae thereof), J² is CH₂. In some embodiments (e.g., including Formulae (XI), (XII), (XIII), and/or (XIV); or sub-formulae thereof), J² is C(O).

In some embodiments (e.g., including Formulae (XI), (XII), (XIII), and/or (XIV); or sub-formulae thereof), J³ is N(R¹⁰). In some embodiments (e.g., including Formulae (XI), (XII), (XIII), and/or (XIV); or sub-formulae thereof), J³ is C(R¹⁰)(R^{10a}). In some embodiments (e.g., including Formulae (XI), (XII), (XIII), and/or (XIV); or sub-formulae thereof), J³ is NH.

In some embodiments of (e.g., of Formulae (I), or sub-formulae thereof), J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of (e.g., of Formulae (I), or sub-formulae thereof), J³ is CH₂ and R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶) SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}. In some embodiments of (e.g., of Formulae (I)), or sub-formulae thereof, J³ is CH₂ and R⁶ is —OR¹⁶. In some embodiments of (e.g., of Formulae (I)), or sub-formulae thereof, J³ is CH₂ and R⁶ is —N(R¹²)(R¹⁶).

In some embodiments (e.g., including Formulae (XI), (XII), (XIII), and/or (XIV); or sub-formulae thereof), J¹ is N and J² is C(R⁹)(R^{9a}). In some embodiments (e.g., including Formulae (XI), (XII), (XIII), and/or (XIV); or sub-formulae thereof), J¹ is C(R⁸) and J² is C(R⁹)(R^{9a}). In some embodiments (e.g., including Formulae (XI), (XII), (XIII), and/or (XIV); or sub-formulae thereof), J¹ is C and J² is C(R⁹). In some embodiments (e.g., including Formulae (XI), (XII), (XIII), and/or (XIV); or sub-formulae thereof), J¹ is N and J² is C(R⁹)(R^{9a}). In some embodiments (e.g., including Formulae (XI), (XII), (XIII), and/or (XIV); or sub-formulae thereof), J¹ is C(R⁸) and J² is C(R⁹)(R^{9a}). In some embodiments (e.g., including Formulae (XI), (XII), (XIII), and/or (XIV); or sub-formulae thereof), J¹ is C and J² is C(R⁹).

In some embodiments, R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, when J² is N(R⁹); R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments, when J² is N(R⁹); R⁹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

In some embodiments, R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, when J³ is N(R¹⁰); R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}. In some embodiments, when J³ is N(R¹⁰); R¹⁰ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}.

In some embodiments (e.g., including Formulae (XI), (XII), (XIII), and/or (XIV); or sub-formulae thereof), W¹ is N. In some embodiments (e.g., including Formulae (XI), (XII), (XIII), and/or (XIV); or sub-formulae thereof), W¹ is N(R¹). In some embodiments (e.g., including Formulae (XI), (XII), (XIII), and/or (XIV); or sub-formulae thereof), W¹ is O. In some embodiments (e.g., including Formulae (XI), (XII), (XIII), and/or (XIV); or sub-formulae thereof), W¹ is C(R¹). In some embodiments (e.g., including Formulae (XI), (XII), (XIII), and/or (XIV); or sub-formulae thereof), W¹ is C(R¹)(R^{1a}). In some embodiments (e.g., including Formulae (XI), (XII), (XIII), and/or (XIV); or sub-formulae thereof), W¹ is C(O). In some embodiments (e.g., including Formulae (XI), (XII), (XIII), and/or (XIV); or sub-formulae thereof), W¹ is S(O). In some embodiments (e.g., including Formulae (XI), (XII), (XIII), and/or (XIV); or sub-formulae thereof), W¹ is S(O)₂. In some embodiments (e.g., including Formulae (XI), (XII), (XIII), and/or (XIV); or sub-formulae thereof), W¹ is S(O)(NR¹). In some embodiments (e.g., including Formulae (XI), (XII), (XIII), and/or (XIV); or sub-formulae thereof), W¹ is S(O)(R¹). In some embodiments (e.g., including Formulae (XI), (XII), (XIII), and/or (XIV); or sub-formulae thereof), W¹ is P(O)(R¹).

In some embodiments (e.g., including Formulae (XI), (XII), (XIII), and/or (XIV); or sub-formulae thereof), R¹ is hydrogen. In some embodiments (e.g., including Formulae (XI), (XII), (XIII), and/or (XIV); or sub-formulae thereof), R¹ is halogen. In some embodiments (e.g., including Formulae (XI), (XII), (XIII), and/or (XIV); or sub-formulae thereof), R¹ is —CN. In some embodiments (e.g., including Formulae (XI), (XII), (XIII), and/or (XIV); or sub-formulae thereof), R¹ is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R¹ is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R¹ is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20a}.

In some embodiments, R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, when W¹ is N(R¹), S(O)(NR¹), S(O)(R¹), or P(O)(R¹); R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, when W¹ is N(R¹), S(O)(NR¹), S(O)(R¹), or P(O)(R¹); R¹ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments, R¹ is —CN. In some embodiments, R¹ is —F. In some embodiments, R¹ is —CF₃. In some embodiments, R¹ is —OCH₃. In some embodiments, R¹ is cyclopropyl. In some embodiments, R¹ is —OCH₂CF₃. In some embodiments, R¹ is —CH₃.

In some embodiments (e.g., including Formulae (XI), (XII), (XIII), and/or (XIV); or sub-formulae thereof), R^{1a} is hydrogen. In some embodiments (e.g., including Formulae (XI), (XII), (XIII), and/or (XIV); or sub-formulae thereof), R^{1a} is halogen. In some embodiments (e.g., including Formulae (XI), (XII), (XIII), and/or (XIV); or sub-formulae thereof), R^{1a} is —CN. In some embodiments (e.g., including Formulae (XI), (XII), (XIII), and/or (XIV); or sub-formulae thereof), R^{1a} is C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R^{1a} is —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R^{1a} is C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20a}.

In some embodiments (e.g., including Formulae (XI), (XII), (XIII), and/or (XIV); or sub-formulae thereof), R¹ and R^{1a} are combined to form a C₃₋₆cycloalkyl. In some embodiments (e.g., including Formulae (XI), (XII), (XIII), and/or (XIV); or sub-formulae thereof), R¹ and R^{1a} are combined to form a C₂₋₉heterocycloalkyl. In some embodiments (e.g., including Formulae (XI), (XII), (XIII), and/or (XIV); or sub-formulae thereof), R¹ and R^{1a} are combined to form a C₃₋₆cycloalkyl substituted with one, two, or three R^{20a}. In some embodiments (e.g., including Formulae (XI), (XII), (XIII), and/or (XIV); or sub-formulae thereof), R¹ and R^{1a} are combined to form a C₂₋₉heterocycloalkyl substituted with one, two, or three R^{20a}.

In some embodiments (e.g., including Formulae (XI), (XII), (XIII), and/or (XIV); or sub-formulae thereof), W² is N. In some embodiments (e.g., including Formulae (XI), (XII), (XIII), and/or (XIV); or sub-formulae thereof), W² is N(R²). In some embodiments (e.g., including Formulae (XI), (XII), (XIII), and/or (XIV); or sub-formulae thereof), W² is O. In some embodiments (e.g., including Formulae (XI), (XII), (XIII), and/or (XIV); or sub-formulae thereof), W² is C(R²). In some embodiments (e.g., including Formulae (XI), (XII), (XIII), and/or (XIV); or sub-formulae thereof), W² is C(R²)(R^{2a}). In some embodiments (e.g., including Formulae (XI), (XII), (XIII), and/or (XIV); or sub-formulae thereof), W² is C(O). In some embodiments (e.g., including Formulae (XI), (XII), (XIII), and/or (XIV); or sub-formulae thereof), W² is S(O). In some embodiments (e.g., including Formulae (XI), (XII), (XIII), and/or (XIV); or sub-formulae thereof), W² is S(O)₂. In some embodiments (e.g., including Formulae (XI), (XII), (XIII), and/or (XIV); or sub-formulae thereof), W² is S(O)(NR²). In some embodiments (e.g., including Formulae (XI), (XII), (XIII), and/or (XIV); or sub-formulae thereof), W² is S(O)(R²). In some embodiments (e.g., including Formulae (XI), (XII), (XIII), and/or (XIV); or sub-formulae thereof), W² is P(O)(R²).

In some embodiments (e.g., including Formulae (XI), (XII), (XIII), and/or (XIV); or sub-formulae thereof), R^{3a} is independently hydrogen. In some embodiments (e.g., including Formulae (XI), (XII), (XIII), and/or (XIV); or sub-formulae thereof), R^{3a} is independently halogen. In some embodiments (e.g., including Formulae (XI), (XII), (XIII), and/or (XIV); or sub-formulae thereof), R^{3a} is independently —CN. In some embodiments (e.g., including Formulae (XI), (XII), (XIII), and/or (XIV); or sub-formulae thereof), R^{3a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments (e.g., including Formulae (XI), (XII), (XIII), and/or (XIV); or sub-formulae thereof), R^{3a} is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R^{3a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20c}.

In some embodiments (e.g., including Formulae (XI), (XII), (XIII), and/or (XIV); or sub-formulae thereof), R³ and R^{3a} are combined to form a C₃₋₆cycloalkyl. In some embodiments (e.g., including Formulae (XI), (XII), (XIII), and/or (XIV); or sub-formulae thereof), R³ and R^{3a} are combined to form a C₂₋₉heterocycloalkyl. In some embodiments (e.g., including Formulae (XI), (XII), (XIII), and/or (XIV); or sub-formulae thereof), R³ and R^{3a} are combined to form a C₃₋₆cycloalkyl substituted with one, two, or three R^{20c}. In some embodiments (e.g., including Formulae (XI), (XII), (XIII), and/or (XIV); or sub-formulae thereof), R³ and R^{3a} are combined to form a C₂₋₉heterocycloalkyl substituted with one, two, or three R^{20c}.

In some embodiments (e.g., including Formulae (XI), (XII), (XIII), and/or (XIV); or sub-formulae thereof), R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}.

In some embodiments (e.g., including Formulae (XI), (XII), (XIII), and/or (XIV); or sub-formulae thereof), R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}. In some embodiments, R³ is —CN. In some embodiments, R³ is —F. In some embodiments, R³ is —CF₃. In some embodiments, R³ is —OCH₃. In some embodiments, R³ is cyclopropyl. In some embodiments, R³ is —OCH₂CF₃. In some embodiments, R³ is —CH₃.

In some embodiments (e.g., including Formulae (XI), (XII), (XIII), and/or (XIV); or sub-formulae thereof), W⁴ is N. In some embodiments (e.g., including Formulae (XI), (XII), (XIII), and/or (XIV); or sub-formulae thereof), W⁴ is N(R⁴). In some embodiments (e.g., including Formulae (XI), (XII), (XIII), and/or (XIV); or sub-formulae thereof), W⁴ is O. In some embodiments (e.g., including Formulae (XI), (XII), (XIII), and/or (XIV); or sub-formulae thereof), W⁴ is C(R⁴). In some embodiments (e.g., including Formulae (XI), (XII), (XIII), and/or (XIV); or sub-formulae thereof), W⁴ is C(R⁴)(R^{4a}). In some embodiments (e.g., including Formulae (XI), (XII), (XIII), and/or (XIV); or sub-formulae thereof), W⁴ is C(O). In some embodiments (e.g., including Formulae (XI), (XII), (XIII), and/or (XIV); or sub-formulae thereof), W⁴ is S(O). In some embodiments (e.g., including Formulae (XI), (XII), (XIII), and/or (XIV); or sub-formulae thereof), W⁴ is S(O)₂. In some embodiments (e.g., including Formulae (XI), (XII), (XIII), and/or (XIV); or sub-formulae thereof), W⁴ is S(O)(NR⁴). In some embodiments (e.g., including Formulae (XI), (XII), (XIII), and/or (XIV); or sub-formulae thereof), W⁴ is S(O)(R⁴). In some embodiments (e.g., including Formulae (XI), (XII), (XIII), and/or (XIV); or sub-formulae thereof), W⁴ is P(O)(R⁴).

In some embodiments (e.g., including Formulae (XI), (XII), (XIII), and/or (XIV); or sub-formulae thereof), R⁴ is independently hydrogen. In some embodiments (e.g., including Formulae (XI), (XII), (XIII), and/or (XIV); or sub-formulae thereof), R⁴ is independently halogen. In some embodiments (e.g., including Formulae (XI), (XII), (XIII), and/or (XIV); or sub-formulae thereof), R⁴ is independently —CN. In some embodiments (e.g., including Formulae (XI), (XII), (XIII), and/or (XIV); or sub-formulae thereof), R⁴ is independently C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R⁴ is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R⁴ is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20d}. In some embodiments (e.g., including Formulae (XI), (XII), (XIII), and/or (XIV); or sub-formulae thereof), R⁴ is independently C₁₋₆ alkyl. In some embodiments (e.g., including Formulae (XI), (XII), (XIII), and/or (XIV); or sub-formulae thereof), R⁴ is independently C₁₋₄alkyl. In some embodiments (e.g., including Formulae (XI), (XII), (XIII), and/or (XIV); or sub-formulae thereof), R⁴ is independently C₁₋₃alkyl. In some embodiments (e.g., including Formulae (XI), (XII), (XIII), and/or (XIV); or sub-formulae thereof), R⁴ is independently C₁₋₂alkyl. In some embodiments (e.g., including Formulae (XI), (XII), (XIII), and/or (XIV); or sub-formulae thereof), R⁴ is independently methyl. In some embodiments, R⁴ is —CN. In some embodiments, R⁴ is —F. In some embodiments, R⁴ is —CF₃. In some embodiments, R⁴ is —OCH₃. In some embodiments, R⁴ is cyclopropyl. In some embodiments, R⁴ is —OCH₂CF₃. In some embodiments, R⁴ is —CH₃.

In some embodiments, R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}. In some embodiments, R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}. In some embodiments, when W⁴ is N(R⁴), S(O)(NR⁴), S(O)(R⁴), or P(O)(R⁴). R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R². In some embodiments, when W⁴ is N(R⁴), S(O)(NR⁴), S(O)(R⁴), or P(O)(R⁴); R⁴ is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}.

In some embodiments (e.g., including Formulae (XI), (XII), (XIII), and/or (XIV); or sub-formulae thereof), R^{4a} is independently hydrogen. In some embodiments (e.g., including Formulae (XI), (XII), (XIII), and/or (XIV); or sub-formulae thereof), R^{4a} is independently halogen. In some embodiments (e.g., including Formulae (XI), (XII), (XIII), and/or (XIV); or sub-formulae thereof), R^{4a} is independently —CN. In some embodiments (e.g., including Formulae (XI), (XII), (XIII), and/or (XIV); or sub-formulae thereof), R^{4a} is independently C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl. In some embodiments (e.g., including Formulae (XI), (XII), (XIII), and/or (XIV); or sub-formulae thereof), R^{4a} is independently —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³). In some embodiments (e.g., including Formulae (XI), (XII), (XIII), and/or (XIV); or sub-formulae thereof), R^{4a} is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl substituted with one, two, or three R^{20d}. In some embodiments (e.g., including Formulae (XI), (XII), (XIII), and/or (XIV); or sub-formulae thereof), R^{4a} is independently C₁₋₆ alkyl. In some embodiments (e.g., including Formulae (XI), (XII), (XIII), and/or (XIV); or sub-formulae thereof), R^{4a} is independently C₁₋₄alkyl. In some embodiments (e.g., including Formulae (XI), (XII), (XIII), and/or (XIV); or sub-formulae thereof), R^{4a} is independently C₁₋₃alkyl. In some embodiments (e.g., including Formulae (XI), (XII), (XIII), and/or (XIV); or sub-formulae thereof), R^{4a} is independently C₁₋₂alkyl. In some embodiments (e.g., including Formulae (XI), (XII), (XIII), and/or (XIV); or sub-formulae thereof), R^{4a} is independently methyl.

In some embodiments (e.g., including Formulae (XI), (XII), (XIII), and/or (XIV); or sub-formulae thereof), R⁴ and R^{4a} are combined to form a C₃₋₆cycloalkyl. In some embodiments (e.g., including Formulae (XI), (XII), (XIII), and/or (XIV); or sub-formulae thereof), R⁴ and R^{4a} are combined to form a C₂₋₉heterocycloalkyl. In some embodiments (e.g., including Formulae (XI), (XII), (XIII), and/or (XIV); or sub-formulae thereof), R⁴ and R^{4a} are combined to form a C₃₋₆cycloalkyl substituted with one, two, or three R^{20d}. In some embodiments (e.g., including Formulae (XI), (XII), (XIII), and/or (XIV); or sub-formulae thereof), R⁴ and R^{4a} are combined to form a C₂₋₉heterocycloalkyl substituted with one, two, or three R^{20d}.

In some embodiments (e.g., including Formulae (XI), (XII), (XIII), and/or (XIV); or sub-formulae thereof), W⁵ is N. In some embodiments (e.g., including Formulae (XI), (XII), (XIII), and/or (XIV); or sub-formulae thereof), W⁵ is C. In some embodiments (e.g., including Formulae (XI), (XII), (XIII), and/or (XIV); or sub-formulae thereof), W⁵ is CH.

In some embodiments (e.g., including Formulae (XI), (XII), (XIII), and/or (XIV); or sub-formulae thereof), W⁶ is N. In some embodiments (e.g., including Formulae (XI), (XII), (XIII), and/or (XIV); or sub-formulae thereof), W⁶ is C. In some embodiments (e.g., including Formulae (XI), (XII), (XIII), and/or (XIV); or sub-formulae thereof), W⁶ is CH.

In some embodiments (e.g., including Formulae (XI), (XII), (XIII), and/or (XIV); or sub-formulae thereof), at least one of W¹, W², W³, W⁴, W⁵, and W⁶ is N, N(R¹), N(R²), N(R³), or N(R⁴). In some embodiments (e.g., including Formulae (XI), (XII), (XIII), and/or (XIV); or sub-formulae thereof), at least one of W¹, W², W³, and W⁴ is N, N(R¹), N(R²), N(R³), or N(R⁴).

In some embodiments (e.g., including Formulae (XI); or sub-formulae thereof), two Ring A [symbol] are double bonds. In some embodiments (e.g., including Formulae (XI); or sub-formulae thereof), three Ring A [symbol] are double bonds. In some embodiments of Formulae (XIII), or sub-formulae thereof, no Ring A [symbol] are double bonds. In some embodiments of Formulae (XIII), or sub-formulae thereof, one Ring A [symbol] is a double bond. In some embodiments of Formulae (XIII), or sub-formulae thereof, two Ring A [symbol] are double bonds.

In some embodiments of Formula X(III), or sub-formulae thereof, W³ is C(O), S(O), S(O)₂, S(O)(NR³), S(O)(R³), or P(O)(R³); W⁴ is N(R⁴); W¹ is C(R¹); and W² is C(R²). In some embodiments of Formula (XIII), or sub-formulae thereof, W¹ is C(O), S(O), S(O)₂, S(O)(NR¹), S(O)(R¹), or P(O)(R¹); W² is N(R²); W³ is C(R³)(R^{3a}); and W⁴ is C(R⁴)(R^{4a}). In some embodiments of Formula (XIII), or sub-formulae thereof, W³ is C(O), S(O), S(O)₂, S(O)(NR³), S(O)(R³), or P(O)(R³); W⁴ is N(R⁴); W² is N(R²); and W¹ is C(R¹)(R^{1a}). In some embodiments of Formula (XIII), or sub-formulae thereof, W³ is C(O), S(O), S(O)₂, S(O)(NR³), S(O)(R³), or P(O)(R³); W² is N(R²); W¹ is C(R¹)(R^{1a}); and W⁴ is C(R⁴)(R^{4a}). In some embodiments of Formula (XIII), or sub-formulae thereof, W³ is C(O), S(O), S(O)₂, S(O)(NR³), S(O)(R³), or P(O)(R³); W² is N(R²); W¹ is C(R¹); and W⁴ is N. In some embodiments of Formula (XIII), or sub-formulae thereof, W³ is C(O), S(O), S(O)₂, S(O)(NR³), S(O)(R³), or P(O)(R³); W² is N(R²); W¹ is C(R¹); and W⁴ is C(R⁴). In some embodiments of Formula (XIII), or sub-formulae thereof, W⁴ is C(O), S(O), S(O)₂, S(O)(NR⁴), S(O)(R⁴), or P(O)(R⁴); W³ is N(R³); W¹ is N; and W² is C(R²). In some embodiments of Formula (XIII), or sub-formulae thereof, W⁴ is C(O), S(O), S(O)₂, S(O)(NR⁴), S(O)(R⁴), or P(O)(R⁴); W³ is N(R³); W¹ is C(R¹); and W² is C(R²). In some embodiments of Formula (XIII), or sub-formulae thereof, W³ is C(O), S(O), S(O)₂, S(O)(NR³), S(O)(R³), or P(O)(R³); W⁴ is N(R⁴); W² is C(R²); and W¹ is C(R¹). In some embodiments of Formula (XIII), or sub-formulae thereof, W¹ is C(O), S(O), S(O)₂, S(O)(NR¹), S(O)(R¹), or P(O)(R¹); W² is N(R²); W³ is C(R³); and W⁴ is C(R⁴). In some embodiments of Formula (XIII), or sub-formulae thereof, W¹ is C(O), S(O), S(O)₂, S(O)(NR¹), S(O)(R¹), or P(O)(R¹); W² is N(R²); W³ is C(R³); and W⁴ is N. In some embodiments of Formula (XIII), or sub-formulae thereof, W⁴ is C(O), S(O), S(O)₂, S(O)(NR⁴), S(O)(R⁴), or P(O)(R⁴); W³ is C(R³)(R^{3a}); W¹ is O; and W² is C(R²)(R^{2a}). In some embodiments of Formula (XIII), or sub-formulae thereof, W⁴ is C(O), S(O), S(O)₂, S(O)(NR⁴), S(O)(R⁴), or P(O)(R⁴); W³ is C(R³); W¹ is O; and W² is C(R²). In some embodiments of Formula (XIII), or sub-formulae thereof, W¹ is C(O), S(O), S(O)₂, S(O)(NR¹), S(O)(R¹), or P(O)(R¹); W³ is C(R³)(R^{3a}); W⁴ is O; and W² is C(R²)(R^{2a}). In some embodiments of Formula (XIII), or sub-formulae thereof, W¹ is C(O), S(O), S(O)₂, S(O)(NR¹), S(O)(R¹), or P(O)(R¹); W³ is C(R³); W⁴ is O; and W² is C(R²).

In some embodiments (e.g., including Formulae (XI), (XII), (XIII), and/or (XIV); or sub-formulae thereof), W¹ is C(R¹); W² is C(R²); W³ is N; and W⁴ is N. In some embodiments (e.g., including Formulae (XI), (XII), (XIII), and/or (XIV); or sub-formulae thereof), W¹ is C(R¹); W² is C(R²); W³ is C(R³); and W⁴ is N. In some embodiments (e.g., including Formulae (XI), (XII), (XIII), and/or (XIV); or sub-formulae thereof), W¹ is C(R¹); W² is C(R²); W³ is N; and W⁴ is C(R⁴). In some embodiments (e.g., including Formulae (XI), (XII), (XIII), and/or (XIV); or sub-formulae thereof), W¹ is N; W² is C(R²); W³ is C(R³); and W⁴ is C(R⁴).

In some embodiments (e.g., including Formulae (XI), (XII), (XIII), and/or (XIV); or sub-formulae thereof), W¹ is N, W² is C(R²), W³ is C(R³), and W⁴ is C(R⁴). In some embodiments (e.g., including Formulae (XI), (XII), (XIII), and/or (XIV); or sub-formulae thereof), W¹ is C(R¹), W² is N, W³ is C(R³), and W⁴ is C(R⁴). In some embodiments (e.g., including Formulae (XI), (XII), (XIII), and/or (XIV); or sub-formulae thereof), W¹ is C(R¹), W² is C(R²), W³ is N, and W⁴ is C(R⁴). In some embodiments (e.g., including Formulae (XI), (XII), (XIII), and/or (XIV); or sub-formulae thereof), W¹ is C(R¹), W² is C(R²), W³ is C(R³), and W⁴ is N. In some embodiments (e.g., including Formulae (XI), (XII), (XIII), and/or (XIV); or sub-formulae thereof), W¹ is C(R¹) and W² is C(R²). In some embodiments (e.g., including Formulae (XI), (XII), (XIII), and/or (XIV); or sub-formulae thereof), W³ is N(R³) and W⁴ is C(O). In some embodiments (e.g., including Formulae (XI), (XII), (XIII), and/or (XIV); or sub-formulae thereof), W³ is C(O) and W⁴ is N(R⁴). In some embodiments (e.g., including Formulae (XI), (XII), (XIII), and/or (XIV); or sub-formulae thereof), W³ is O and W⁴ is C(R⁴)(R^{4a}). In some embodiments (e.g., including Formulae (XI), (XII), (XIII), and/or (XIV); or sub-formulae thereof), W¹ is C(R¹)(R^{1a}), W² is O, W³ is C(R³), and W⁴ is C(R⁴). In some embodiments (e.g., including Formulae (XI), (XII), (XIII), and/or (XIV); or sub-formulae thereof), W¹ is CO, N(R¹) or C(R¹)(R^{1a}); W² is CO, N(R²), or C(R²)(R^{a}); W³ is N, or C(R³); and W⁴ is N, or C(R⁴).

In some embodiments (e.g., including Formulae (XI), (XII), (XIII), and/or (XIV); or sub-formulae thereof), W³ and W⁴ are connected by a double bond. In some embodiments (e.g., including Formulae (XI), (XII), (XIII), and/or (XIV); or sub-formulae thereof), W¹ is is N(R¹) and W³ and W⁴ are connected by a double bond. In some embodiments (e.g., including Formulae (XI), (XII), (XIII), and/or (XIV); or sub-formulae thereof), W² is N(R²) and W¹ is C(R¹)(R^{1a}). In some embodiments (e.g., including Formulae (XI), (XII), (XIII), and/or (XIV); or sub-formulae thereof), W⁴ is N(R⁴) and W¹ and W² are connected by a double bond. In some embodiments (e.g., including Formulae (XI), (XII), (XIII), and/or (XIV); or sub-formulae thereof), W² is C(R²)(R²) and W³ is C(R³)(R^{3a}). In some embodiments (e.g., including Formulae (XI), (XII), (XIII), and/or (XIV); or sub-formulae thereof), W² is C(R²) and W³ is C(R³). In some embodiments of Formula (III), or sub-formulae thereof, W⁴ is C(O), S(O), S(O)₂, S(O)(NR⁴), S(O)(R⁴), or P(O)(R⁴); W³ is N; and W² is C(R²). In some embodiments of Formula (III), or sub-formulae thereof, W⁴ is C(O), S(O), S(O)₂, S(O)(NR⁴), S(O)(R⁴), or P(O)(R⁴); W² is N; and W³ is C(R³). In some embodiments of Formula (XIII), or sub-formulae thereof, W⁴ is C(O), S(O), S(O)₂, S(O)(NR⁴), S(O)(R⁴), or P(O)(R⁴); W³ is C(R³); and W² is C(R²). In some embodiments of Formula (XIII), or sub-formulae thereof, W⁴ is C(O), S(O), S(O)₂, S(O)(NR⁴), S(O)(R⁴), or P(O)(R⁴); W³ is N; and W² is N. In some embodiments of Formula (XIII), or sub-formulae thereof, W¹ is C(O), S(O), S(O)₂, S(O)(NR¹), S(O)(R¹), or P(O)(R¹); W³ is N; and W² is C(R²). In some embodiments of Formula (XIII), or sub-formulae thereof, W¹ is C(O), S(O), S(O)₂, S(O)(NR¹), S(O)(R¹), or P(O)(R¹); W² is N; and W³ is C(R³). In some embodiments of Formula (XIII), or sub-formulae thereof, W¹ is C(O), S(O), S(O)₂, S(O)(NR¹), S(O)(R¹), or P(O)(R¹); W³ is C(R³); and W² is C(R²). In some embodiments of Formula (XIII), or sub-formulae thereof, W¹ is C(O), S(O), S(O)₂, S(O)(NR¹), S(O)(R¹), or P(O)(R¹); W³ is N; and W² is N.

In some embodiments (e.g., including Formulae (XI), (XII), (XIII), and/or (XIV); or sub-formulae thereof), R¹² is independently hydrogen. In some embodiments (e.g., including Formulae (XI), (XII), (XIII), and/or (XIV); or sub-formulae thereof), R¹² is independently C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, or C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments (e.g., including Formulae (XI), (XII), (XIII), and/or (XIV); or sub-formulae thereof), R¹² is independently C₁₋₆alkyl optionally substituted with one, two, or three R^{20k}. In some embodiments (e.g., including Formulae (XI), (XII), (XIII), and/or (XIV); or sub-formulae thereof), R¹² is independently C₁₋₆alkyl. In some embodiments (e.g., including Formulae (XI), (XII), (XIII), and/or (XIV); or sub-formulae thereof), R¹² is independently C₁₋₄alkyl. In some embodiments (e.g., including Formulae (XI), (XII), (XIII), and/or (XIV); or sub-formulae thereof), R¹² is independently C₁₋₃alkyl. In some embodiments (e.g., including Formulae (XI), (XII), (XIII), and/or (XIV); or sub-formulae thereof), R¹² is independently C₁₋₂alkyl. In some embodiments (e.g., including Formulae (XI), (XII), (XIII), and/or (XIV); or sub-formulae thereof), R¹² is methyl.

In some embodiments (e.g., including Formulae (XI), (XII), (XIII), and/or (XIV); or sub-formulae thereof), R^{12a} is independently hydrogen. In some embodiments (e.g., including Formulae (XI), (XII), (XIII), and/or (XIV); or sub-formulae thereof), R^{12a} is independently C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments (e.g., including Formulae (XI), (XII), (XIII), and/or (XIV); or sub-formulae thereof), R^{12a} is independently C₁₋₆alkyl optionally substituted with one, two, or three R^{20k}. In some embodiments (e.g., including Formulae (XI), (XII), (XIII), and/or (XIV); or sub-formulae thereof), R^{12a} is independently C₁₋₆alkyl. In some embodiments (e.g., including Formulae (XI), (XII), (XIII), and/or (XIV); or sub-formulae thereof), R^{12a} is independently C₁₋₄alkyl. In some embodiments (e.g., including Formulae (XI), (XII), (XIII), and/or (XIV); or sub-formulae thereof), R¹² is independently C₁₋₃alkyl. In some embodiments (e.g., including Formulae (XI), (XII), (XIII), and/or (XIV); or sub-formulae thereof), R^{12a} is independently C₁₋₂alkyl. In some embodiments (e.g., including Formulae (XI), (XII), (XIII), and/or (XIV); or sub-formulae thereof), R^{12a} is methyl.

In some embodiments (e.g., including Formulae (XI), (XII), (XIII), and/or (XIV); or sub-formulae thereof), R¹² is independently hydrogen. In some embodiments (e.g., including Formulae (XI), (XII), (XIII), and/or (XIV); or sub-formulae thereof), R¹² is independently C₁₋₆alkyl. In some embodiments (e.g., including Formulae (XI), (XII), (XIII), and/or (XIV); or sub-formulae thereof), R¹² is independently C₂₋₆alkenyl. In some embodiments (e.g., including Formulae (XI), (XII), (XIII), and/or (XIV); or sub-formulae thereof), R¹² is independently C₂₋₆alkynyl. In some embodiments (e.g., including Formulae (XI), (XII), (XIII), and/or (XIV); or sub-formulae thereof), R¹² is independently C₃₋₆cycloalkyl. In some embodiments (e.g., including Formulae (XI), (XII), (XIII), and/or (XIV); or sub-formulae thereof), R¹² is independently —CH₂—C₃₋₆cycloalkyl. In some embodiments (e.g., including Formulae (XI), (XII), (XIII), and/or (XIV); or sub-formulae thereof), R¹² is independently C₂₋₉heterocycloalkyl. In some embodiments (e.g., including Formulae (XI), (XII), (XIII), and/or (XIV); or sub-formulae thereof), R¹² is independently —CH₂—C₂₋₉heterocycloalkyl. In some embodiments (e.g., including Formulae (XI), (XII), (XIII), and/or (XIV); or sub-formulae thereof), R¹² is independently C₆₋₁₀aryl. In some embodiments (e.g., including Formulae (XI), (XII), (XIII), and/or (XIV); or sub-formulae thereof), R¹² is independently —CH₂—C₆₋₁₀aryl. In some embodiments (e.g., including Formulae (XI), (XII), (XIII), and/or (XIV); or sub-formulae thereof), R¹² is independently C₁₋₉heteroaryl.

In some embodiments (e.g., including Formulae (XI), (XII), (XIII), and/or (XIV); or sub-formulae thereof), R¹² is independently C₁₋₆alkyl substituted with one, two, or three R^{20k}. In some embodiments (e.g., including Formulae (XI), (XII), (XIII), and/or (XIV); or sub-formulae thereof), R¹² is independently C₂₋₆alkenyl substituted with one, two, or three R^{20k}. In some embodiments (e.g., including Formulae (XI), (XII), (XIII), and/or (XIV); or sub-formulae thereof), R¹² is independently C₂₋₆alkynyl substituted with one, two, or three R^{20k}. In some embodiments (e.g., including Formulae (XI), (XII), (XIII), and/or (XIV); or sub-formulae thereof), R¹² is independently C₃₋₆cycloalkyl substituted with one, two, or three R^{20k}. In some embodiments (e.g., including Formulae (XI), (XII), (XIII), and/or (XIV); or sub-formulae thereof), R¹² is independently —CH₂—C₃₋₆cycloalkyl substituted with one, two, or three R^{20k}. In some embodiments (e.g., including Formulae (XI), (XII), (XIII), and/or (XIV); or sub-formulae thereof), R¹² is independently C₂₋₉heterocycloalkyl substituted with one, two, or three R^{20k}. In some embodiments (e.g., including Formulae (XI), (XII), (XIII), and/or (XIV); or sub-formulae thereof), R¹² is independently —CH₂—C₂₋₉heterocycloalkyl substituted with one, two, or three R^{20k}. In some embodiments (e.g., including Formulae (XI), (XII), (XIII), and/or (XIV); or sub-formulae thereof), R¹² is independently C₆₋₁₀aryl substituted with one, two, or three R^{20k}. In some embodiments (e.g., including Formulae (XI), (XII), (XIII), and/or (XIV); or sub-formulae thereof), R¹² is independently —CH₂—C₆₋₁₀aryl substituted with one, two, or three R^{20k}. In some embodiments (e.g., including Formulae (XI), (XII), (XIII), and/or (XIV); or sub-formulae thereof), R¹² is independently C₁₋₉heteroaryl substituted with one, two, or three R^{20k}.

In some embodiments (e.g., including Formulae (XI), (XII), (XIII), and/or (XIV); or sub-formulae thereof), R^{12a} is independently hydrogen. In some embodiments (e.g., including Formulae (XI), (XII), (XIII), and/or (XIV); or sub-formulae thereof), R^{12a} is independently C₁₋₆alkyl. In some embodiments (e.g., including Formulae (XI), (XII), (XIII), and/or (XIV); or sub-formulae thereof), R^{12a} is independently C₂₋₆alkenyl. In some embodiments (e.g., including Formulae (XI), (XII), (XIII), and/or (XIV); or sub-formulae thereof), R^{12a} is independently C₂₋₆alkynyl. In some embodiments (e.g., including Formulae (XI), (XII), (XIII), and/or (XIV); or sub-formulae thereof), R^{12a} is independently C₃₋₆cycloalkyl. In some embodiments (e.g., including Formulae (XI), (XII), (XIII), and/or (XIV); or sub-formulae thereof), R^{12a} is independently —CH₂—C₃₋₆cycloalkyl. In some embodiments (e.g., including Formulae (XI), (XII), (XIII), and/or (XIV); or sub-formulae thereof), R^{12a} is independently —CH₂—C₂₋₉heterocycloalkyl. In some embodiments (e.g., including Formulae (XI), (XII), (XIII), and/or (XIV); or sub-formulae thereof), R^{12a} is independently C₆₋₁₀aryl. In some embodiments (e.g., including Formulae (XI), (XII), (XIII), and/or (XIV); or sub-formulae thereof), R^{12a} is independently —CH₂—C₆₋₁₀aryl. In some embodiments (e.g., including Formulae (XI), (XII), (XIII), and/or (XIV); or sub-formulae thereof), R^{12a} is independently C₁₋₉heteroaryl. In some embodiments (e.g., including Formulae (XI), (XII), (XIII), and/or (XIV); or sub-formulae thereof), R^{12a} is independently C₁₋₆alkyl substituted with one, two, or three R^{20k}. In some embodiments (e.g., including Formulae (XI), (XII), (XIII), and/or (XIV); or sub-formulae thereof), R^{12a} is independently C₂₋₆alkenyl substituted with one, two, or three R^{20k}. In some embodiments (e.g., including Formulae (XI), (XII), (XIII), and/or (XIV); or sub-formulae thereof), R^{12a} is independently C₂₋₆alkynyl substituted with one, two, or three R^{20k}. In some embodiments (e.g., including Formulae (XI), (XII), (XIII), and/or (XIV); or sub-formulae thereof), R^{12a} is independently C₃₋₆cycloalkyl substituted with one, two, or three R^{20k}. In some embodiments (e.g., including Formulae (XI), (XII), (XIII), and/or (XIV); or sub-formulae thereof), R^{12a} is independently —CH₂—C₃₋₆cycloalkyl substituted with one, two, or three R^{20k}. In some embodiments (e.g., including Formulae (XI), (XII), (XIII), and/or (XIV); or sub-formulae thereof), R^{12a} is independently —CH₂—C₂₋₉heterocycloalkyl substituted with one, two, or three R^{20k}. In some embodiments (e.g., including Formulae (XI), (XII), (XIII), and/or (XIV); or sub-formulae thereof), R^{12a} is independently C₆₋₁₀aryl substituted with one, two, or three R^{20k}. In some embodiments (e.g., including Formulae (XI), (XII), (XIII), and/or (XIV); or sub-formulae thereof), R^{12a} is independently —CH₂—C₆₋₁₀aryl substituted with one, two, or three R^{20k}. In some embodiments (e.g., including Formulae (XI), (XII), (XIII), and/or (XIV); or sub-formulae thereof), R^{12a} is independently C₁₋₉heteroaryl substituted with one, two, or three R^{20k}.

In some embodiments (e.g., including Formulae (XI), (XII), (XIII), and/or (XIV); or sub-formulae thereof), R¹³ is independently hydrogen. In some embodiments (e.g., including Formulae (XI), (XII), (XIII), and/or (XIV); or sub-formulae thereof), R¹³ is independently C₁₋₆alkyl. In some embodiments (e.g., including Formulae (XI), (XII), (XIII), and/or (XIV); or sub-formulae thereof), R¹³ is independently C₁₋₆haloalkyl.

In some embodiments (e.g., including Formulae (XI), (XII), (XIII), and/or (XIV); or sub-formulae thereof), R¹² and R¹³, together with the nitrogen to which they are attached, form a C₂₋₉heterocycloalkyl ring. In some embodiments (e.g., including Formulae (XI), (XII), (XIII), and/or (XIV); or sub-formulae thereof), R¹² and R¹³, together with the nitrogen to which they are attached, form a C₂₋₉heterocycloalkyl ring substituted with one, two, or three R^{20k}.

In some embodiments (e.g., including Formulae (XI), (XII), (XIII), and/or (XIV); or sub-formulae thereof), R¹⁴ is independently hydrogen. In some embodiments (e.g., including Formulae (XI), (XII), (XIII), and/or (XIV); or sub-formulae thereof), R¹⁴ is independently C₁₋₆alkyl. In some embodiments (e.g., including Formulae (XI), (XII), (XIII), and/or (XIV); or sub-formulae thereof), R¹⁴ is independently C₁₋₆haloalkyl.

In some embodiments (e.g., including Formulae (XI), (XII), (XIII), and/or (XIV); or sub-formulae thereof), R¹⁵ is independently C₁₋₆alkyl. In some embodiments (e.g., including Formulae (XI), (XII), (XIII), and/or (XIV); or sub-formulae thereof), R¹⁵ is independently C₂₋₆alkenyl. In some embodiments (e.g., including Formulae (XI), (XII), (XIII), and/or (XIV); or sub-formulae thereof), R¹⁵ is independently C₂₋₆alkynyl. In some embodiments (e.g., including Formulae (XI), (XII), (XIII), and/or (XIV); or sub-formulae thereof), R¹⁵ is independently C₃₋₆cycloalkyl. In some embodiments (e.g., including Formulae (XI), (XII), (XIII), and/or (XIV); or sub-formulae thereof), R¹⁵ is independently C₁₋₆heteroalkyl. In some embodiments (e.g., including Formulae (XI), (XII), (XIII), and/or (XIV); or sub-formulae thereof), R¹⁵ is independently C₂₋₉heterocycloalkyl. In some embodiments (e.g., including Formulae (XI), (XII), (XIII), and/or (XIV); or sub-formulae thereof), R¹⁵ is independently C₆₋₁₀aryl. In some embodiments (e.g., including Formulae (XI), (XII), (XIII), and/or (XIV); or sub-formulae thereof), R¹⁵ is independently C₁₋₉heteroaryl. In some embodiments (e.g., including Formulae (XI), (XII), (XIII), and/or (XIV); or sub-formulae thereof), R¹⁵ is independently C₁₋₆alkyl substituted with one, two, or three R^{20k}. In some embodiments (e.g., including Formulae (XI), (XII), (XIII), and/or (XIV); or sub-formulae thereof), R¹⁵ is independently C₂₋₆alkenyl substituted with one, two, or three R^{20k}. In some embodiments (e.g., including Formulae (XI), (XII), (XIII), and/or (XIV); or sub-formulae thereof), R¹⁵ is independently C₂₋₆alkynyl substituted with one, two, or three R^{20k}. In some embodiments (e.g., including Formulae (XI), (XII), (XIII), and/or (XIV); or sub-formulae thereof), R¹⁵ is independently C₃₋₆cycloalkyl substituted with one, two, or three R^{20k}. In some embodiments (e.g., including Formulae (XI), (XII), (XIII), and/or (XIV); or sub-formulae thereof), R¹⁵ is independently C₁₋₆heteroalkyl substituted with one, two, or three R^{20k}. In some embodiments (e.g., including Formulae (XI), (XII), (XIII), and/or (XIV); or sub-formulae thereof), R¹⁵ is independently C₂₋₉heterocycloalkyl substituted with one, two, or three R^{20k}. In some embodiments (e.g., including Formulae (XI), (XII), (XIII), and/or (XIV); or sub-formulae thereof), R¹⁵ is independently C₆₋₁₀aryl substituted with one, two, or three R^{20k}. In some embodiments (e.g., including Formulae (XI), (XII), (XIII), and/or (XIV); or sub-formulae thereof), R¹⁵ is independently C₁₋₉heteroaryl substituted with one, two, or three R^{20k}. In some embodiments (e.g., including Formulae (XI), (XII), (XIII), and/or (XIV); or sub-formulae thereof), R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20k}. In some embodiments (e.g., including Formulae (XI), (XII), (XIII), and/or (XIV); or sub-formulae thereof), R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl. In some embodiments (e.g., including Formulae (XI), (XII), (XIII), and/or (XIV); or sub-formulae thereof), R¹⁵ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20k}.

In some embodiments (e.g., including Formulae (XI), (XII), (XIII), and/or (XIV); or sub-formulae thereof), R¹⁶ is independently —C₁₋₆alkylene-OP(O)(OR^{16a})(OR^{16b}). In some embodiments (e.g., including Formulae (XI), (XII), (XIII), and/or (XIV); or sub-formulae thereof), R¹⁶ is independently —C₁₋₆alkylene-OP(O)(OR^{16a})(OR^{16b}), wherein the C₁₋₆alkylene is substituted with one, two, or three R^{20l}. In some embodiments (e.g., including Formulae (XI), (XII), (XIII), and/or (XIV); or sub-formulae thereof), R¹⁶ is independently —P(O)(OR^{16a})(OR^{16b}).

In some embodiments (e.g., including Formulae (XI), (XII), (XIII), and/or (XIV); or sub-formulae thereof), R¹⁶ is independently-C₁alkylene-OP(O)(OR^{16a})(OR^{16b}). In some embodiments (e.g., including Formulae (XI), (XII), (XIII), and/or (XIV); or sub-formulae thereof), R¹⁶ is independently-C₁alkylene-OP(O)(OR^{16a})(OR^{16b}), wherein the C₁alkylene is substituted with one or two R^{20l}. In some embodiments (e.g., including Formulae (XI), (XII), (XIII), and/or (XIV); or sub-formulae thereof), R¹⁶ is independently-C₂alkylene-OP(O)(OR^{16a})(OR^{16b}). In some embodiments (e.g., including Formulae (XI), (XII), (XIII), and/or (XIV); or sub-formulae thereof), R¹⁶ is independently-C₂alkylene-OP(O)(OR^{16a})(OR^{16b}), wherein the C₂alkylene is substituted with one, two, or three R^{20l}. In some embodiments (e.g., including Formulae (XI), (XII), (XIII), and/or (XIV); or sub-formulae thereof), R¹⁶ is independently-C₃alkylene-OP(O)(OR^{16a})(OR^{61b}). In some embodiments (e.g., including Formulae (XI), (XII), (XIII), and/or (XIV); or sub-formulae thereof), R¹⁶ is independently-C₃alkylene-OP(O)(OR^{16a})(OR^{16b}), wherein the C₃alkylene is substituted with one, two, or three R^{20l}. In some embodiments (e.g., including Formulae (XI), (XII), (XIII), and/or (XIV); or sub-formulae thereof), R¹⁶ is independently-C₄alkylene-OP(O)(OR^{16a})(OR^{16b}). In some embodiments (e.g., including Formulae (XI), (XII), (XIII), and/or (XIV); or sub-formulae thereof), R¹⁶ is independently-C₄alkylene-OP(O)(OR^{16a})(OR^{16b}), wherein the C₄alkylene is substituted with one, two, or three R^{20l}. In some embodiments (e.g., including Formulae (XI), (XII), (XIII), and/or (XIV); or sub-formulae thereof), R¹⁶ is independently-C₅alkylene-OP(O)(OR^{16a})(OR^{16b}). In some embodiments (e.g., including Formulae (XI), (XII), (XIII), and/or (XIV); or sub-formulae thereof), R¹⁶ is independently-C₅alkylene-OP(O)(OR^{16a})(OR^{16b}), wherein the C₅alkylene is substituted with one, two, or three R^{20l}. In some embodiments (e.g., including Formulae (XI), (XII), (XIII), and/or (XIV); or sub-formulae thereof), R¹⁶ is independently-C₆alkylene-OP(O)(OR^{16a})(OR⁶I). In some embodiments (e.g., including Formulae (XI), (XII), (XIII), and/or (XIV); or sub-formulae thereof), R¹⁶ is independently-C₆alkylene-OP(O)(OR^{16a})(OR^{16b}), wherein the C₆alkylene is substituted with one, two, or three R^{20l}.

In some embodiments (e.g., including Formulae (XI), (XII), (XIII), and/or (XIV); or sub-formulae thereof), R^{16a} is independently hydrogen. In some embodiments (e.g., including Formulae (XI), (XII), (XIII), and/or (XIV); or sub-formulae thereof), R^{16a} is independently C₁₋₆alkyl. In some embodiments (e.g., including Formulae (XI), (XII), (XIII), and/or (XIV); or sub-formulae thereof), R^{16a} is independently C₂₋₆alkenyl. In some embodiments (e.g., including Formulae (XI), (XII), (XIII), and/or (XIV); or sub-formulae thereof), R^{16a} is independently C₂₋₆alkynyl. In some embodiments (e.g., including Formulae (XI), (XII), (XIII), and/or (XIV); or sub-formulae thereof), R¹⁶ is independently C₃₋₆cycloalkyl. In some embodiments (e.g., including Formulae (XI), (XII), (XIII), and/or (XIV); or sub-formulae thereof), R^{16a} is independently —CH₂—C₃₋₆cycloalkyl. In some embodiments (e.g., including Formulae (XI), (XII), (XIII), and/or (XIV); or sub-formulae thereof), R^{16a} is independently —CH₂—C₂₋₉heterocycloalkyl. In some embodiments (e.g., including Formulae (XI), (XII), (XIII), and/or (XIV); or sub-formulae thereof), R¹⁶ is independently C₆₋₁₀aryl. In some embodiments (e.g., including Formulae (XI), (XII), (XIII), and/or (XIV); or sub-formulae thereof), R^{16a} is independently —CH₂—C₆₋₁₀aryl. In some embodiments (e.g., including Formulae (XI), (XII), (XIII), and/or (XIV); or sub-formulae thereof), R^{16a} is independently C₁₋₉heteroaryl. In some embodiments (e.g., including Formulae (XI), (XII), (XIII), and/or (XIV); or sub-formulae thereof), R^{16a} is independently C₁₋₆alkyl substituted with one, two, or three R^{20l}. In some embodiments (e.g., including Formulae (XI), (XII), (XIII), and/or (XIV); or sub-formulae thereof), R¹⁶ is independently C₂₋₆alkenyl substituted with one, two, or three R^{20m}.

In some embodiments (e.g., including Formulae (XI), (XII), (XIII), and/or (XIV); or sub-formulae thereof), R^{16a} is independently C₂₋₆alkynyl substituted with one, two, or three R^{20m}. In some embodiments (e.g., including Formulae (XI), (XII), (XIII), and/or (XIV); or sub-formulae thereof), R^{16a} is independently C₃₋₆cycloalkyl substituted with one, two, or three R^{20m}. In some embodiments (e.g., including Formulae (XI), (XII), (XIII), and/or (XIV); or sub-formulae thereof), R¹⁶ is independently —CH₂—C₃₋₆cycloalkyl substituted with one, two, or three R^{20m}. In some embodiments (e.g., including Formulae (XI), (XII), (XIII), and/or (XIV); or sub-formulae thereof), R^{16a} is independently —CH₂—C₂₋₉heterocycloalkyl substituted with one, two, or three R^{20m}. In some embodiments (e.g., including Formulae (XI), (XII), (XIII), and/or (XIV); or sub-formulae thereof), R¹⁶ is independently C₆₋₁₀aryl substituted with one, two, or three R^{20m}. In some embodiments (e.g., including Formulae (XI), (XII), (XIII), and/or (XIV); or sub-formulae thereof), R^{16a} is independently —CH₂—C₆₋₁₀aryl substituted with one, two, or three R^{20m}. In some embodiments (e.g., including Formulae (XI), (XII), (XIII), and/or (XIV); or sub-formulae thereof), R^{16a} is independently C₁₋₉heteroaryl substituted with one, two, or three R^{20m}.

In some embodiments (e.g., including Formulae (XI), (XII), (XIII), and/or (XIV); or sub-formulae thereof), R^{16b} is independently hydrogen. In some embodiments (e.g., including Formulae (XI), (XII), (XIII), and/or (XIV); or sub-formulae thereof), R^{16b} is independently C₁₋₆alkyl. In some embodiments (e.g., including Formulae (XI), (XII), (XIII), and/or (XIV); or sub-formulae thereof), R^{16b} is independently C₂₋₆alkenyl. In some embodiments (e.g., including Formulae (XI), (XII), (XIII), and/or (XIV); or sub-formulae thereof), R^{16b} is independently C₂₋₆alkynyl. In some embodiments (e.g., including Formulae (XI), (XII), (XIII), and/or (XIV); or sub-formulae thereof), R^{16b} is independently C₃₋₆cycloalkyl. In some embodiments (e.g., including Formulae (XI), (XII), (XIII), and/or (XIV); or sub-formulae thereof), R^{16b} is independently —CH₂—C₃₋₆cycloalkyl. In some embodiments (e.g., including Formulae (XI), (XII), (XIII), and/or (XIV); or sub-formulae thereof), R^{16b} is independently —CH₂—C₂₋₉heterocycloalkyl. In some embodiments (e.g., including Formulae (XI), (XII), (XIII), and/or (XIV); or sub-formulae thereof), R^{16b} is independently C₆₋₁₀aryl. In some embodiments (e.g., including Formulae (XI), (XII), (XIII), and/or (XIV); or sub-formulae thereof), R^{16b} is independently —CH₂—C₆₋₁₀aryl. In some embodiments (e.g., including Formulae (XI), (XII), (XIII), and/or (XIV); or sub-formulae thereof), R^{16b} is independently C₁₋₉heteroaryl. In some embodiments (e.g., including Formulae (XI), (XII), (XIII), and/or (XIV); or sub-formulae thereof), R^{16b} is independently C₁₋₆alkyl substituted with one, two, or three R^{20m}. In some embodiments (e.g., including Formulae (XI), (XII), (XIII), and/or (XIV); or sub-formulae thereof), R^{16b} is independently C₂₋₆alkenyl substituted with one, two, or three R^{20m}. In some embodiments (e.g., including Formulae (XI), (XII), (XIII), and/or (XIV); or sub-formulae thereof), R^{16b} is independently C₂₋₆alkynyl substituted with one, two, or three R^{20m}. In some embodiments (e.g., including Formulae (XI), (XII), (XIII), and/or (XIV); or sub-formulae thereof), R^{16b} is independently C₃₋₆cycloalkyl substituted with one, two, or three R^{20m}. In some embodiments (e.g., including Formulae (XI), (XII), (XIII), and/or (XIV); or sub-formulae thereof), R^{16b} is independently —CH₂—C₃₋₆cycloalkyl substituted with one, two, or three R^{20m}. In some embodiments (e.g., including Formulae (XI), (XII), (XIII), and/or (XIV); or sub-formulae thereof), R^{16b} is independently —CH₂—C₂₋₉heterocycloalkyl substituted with one, two, or three R^{20m}. In some embodiments (e.g., including Formulae (XI), (XII), (XIII), and/or (XIV); or sub-formulae thereof), R^{16b} is independently C₆₋₁₀aryl substituted with one, two, or three R^{20m}. In some embodiments (e.g., including Formulae (XI), (XII), (XIII), and/or (XIV); or sub-formulae thereof), R^{16b} is independently —CH₂—C₆₋₁₀aryl substituted with one, two, or three R^{20m}. In some embodiments (e.g., including Formulae (XI), (XII), (XIII), and/or (XIV); or sub-formulae thereof), R^{16b} is independently C₁₋₉heteroaryl substituted with one, two, or three R^{20m}.

Sub-formulae of Formula I, II, III, and/or IV, are those formula having the same number as the parent formula (e.g., I, II, III, and/or IV) followed by one or more letters and/or apostrophes and/or numbers. In embodiments, sub-formulae of Formula I include I-3, Ia-3, Ib-3, Ic-3, Ig-3, Ih-3, Ii-3, I′, Ia′, Ib′, Ic′, Ia′-1, Ib′-1, Ic′-1, Ia′-2, Ib′-2, Ic′-2, I′″, Ia′, Ib′″, Ic′″, Ia′″-1, Ib′″-1, Ic′″-1, Ia′″-2, Ib′″-2, Ic′″-2, I′″, Ia′″, Ib′″, Ic′″, Ia′″-1, Ib′″-1, Ic′″-1, Ia′″-2, Ib′″-2, Ic′″-2, Ia, Ib, Ic, Id, Ie, If, Ig, Ih, Ii, Ij, Ik, Il, Im, In, Io, Ioa, Iob, Ioc, Ip, Ipa, Ipb, Ipc, Iq, Ir, Is, It, Iu, Iv, Iw, Ix, Iy, Iz, Iaa, Iab, and Iac. In embodiments, sub-formulae of Formula II include II-3, IIa, IIb, IIc, IId, lIe, IIf, IIg, IIh, IIi, IIj, and Ilk. In embodiments, sub-formulae of Formula III Include III-3, III′, IIIa′, IIIb′, IIIc′, IIIa′-1, IIIb′-1, IIIc′-1, IIIa′-2, IIIb′-2, IIIc′-2, III″, IIIa″, IIIb″, IIIc″, IIIa″-1, IIIb″-1, IIIc″-1, IIIa″-2, IIIb″- 2, IIIc″-2, III′″, IIIa′″, IIIb′″, IIIc′″, IIIa′″-1, IIIb′″-1, IIIc′″-1, IIIa′″-2, IIIb′″-2, IIIc′″-2, III⁴, IIIa⁴, IIIb⁴, IIIc⁴, IIIa⁴-1, IIIb¹-1, IIIc⁴-1, IIIa⁴-2, IIIb⁴-2, IIIc⁴-2, III′, IIIa⁵, IIIb′, IIIc⁵, IIIa⁵-1, IIIb⁵-1, IIIc⁵-1, IIIa⁵-2, IIIb⁵-2, IIIc⁵-2, IIIa, IIIb, IIIc, IIId, Ie, IIIf, bg, IIIh, IIIi, IIIj, IIIk, IIll, IIIm, IIIn, IIIo, IIIoa, IIIob, IIIoc, IIIp, IIIpa, IIIpb, IIIpc, IIIq, IIIr, IIIs, IIIt, IIIu, IIIy, IIIw, IIIx, lily, and IIIz. In embodiments, sub-formulae of Formula IV Include IVa, IVb, IVc, IVd, IVe, IVf, IVg, IVh, IVi, IVj, IVk, IVI, IVm, IVn, and IVo.

In embodiments, a compound of Formulae (XI) or sub-formulae thereof is understood to include a formula selected from Formulae I-3, Ia-3, Ib-3, Ic-3, Ig-3, Ih-3, Ii-3, I, I′, Ia′, Ib′, Ic′, Ia′-1, Ib′-1, Ic′-1, Ia′-2, Ib′-2, Ic′-2, I′″, Ia′″, Ib′″, Ic″, Ia′″-1, Ib′″-1, Ic′″-1, Ia′″-2, Ib′″-2, Ic′″-2, I′″, Ia′″, Ib′″, Ic′″, Ia′″-1, Ib′″-1, Ic′″-1, Ia′″-2, Ib′″-2, Ic′″-2, Ia, Ib, Ic, Id, Ie, If, Ig, Ih, Ii, Ij, Ik, Il, Im, In, Io, Ioa, Iob, Ioc, Ip, Ipa, Ipb, Ipc, Iq, Ir, Is, It, Iu, Iv, Iw, Ix, Iy, Iz, Iz, Iaa, Iab, and Iac.

In embodiments, a compound of Formulae (XII) or sub-formulae thereof is understood to include a formula selected from Formulae II-3, II, IIa, IIb, IIc, IId, IIe, IIf, IIg, IIh, IIi, IIj, and Ilk.

In embodiments, a compound of Formulae (XIII) or sub-formulae thereof is understood to include a formula selected from Formulae III-3, III, III′, IIIa′, IIIb′, IIIc′, IIIa′-1, IIIb′-1, IIIc′-1, IIIa′-2, IIIb′-2, IIIc′-2, III″, IIIa″, IIIb″, IIIc″, IIIa″-1, IIIb″-1, IIIc″-1, IIIa″-2, IIIb″-2, IIIc″-2, III″, IIIa′″, IIIb′″, IIIc′″, IIIa′″-1, IIIb′″-1, IIIc′″-1, IIIa′″-2, IIIb′″-2, IIIc′″-2, III⁴, IIIa⁴, IIIb⁴, IIIc⁴, IIIa⁴-1, IIIb⁴-1, IIIc⁴-1, IIIa⁴-2, IIIb⁴-2, IIIc⁴-2, III′, IIIa⁵, IIIb⁵, IIIc⁵, IIIa⁵-1, IIlb⁵-1, IIIc⁵-1, IIIa⁵-2, IIIb⁵-2, IIIc⁵-2, IIIa, IIIb, IIIc, IIId, bIle, IIIf, g, IIIh, IIIi, IIIj, IIIk, IIIl, IIIm, IIIn, IIIo, IIIoa, IIIob, IIIoc, IIIp, IIIpa, IIIpb, IIIpc, IIIq, IIIr, IIIs, IIIt, IIIt, IIIu, IIIy, IIIw, IIIx, lIly, and IIIz.

In embodiments, a compound of Formulae (XIV) or sub-formulae thereof is understood to include a formula selected from Formulae IV, IVa, IVb, IVc, IVd, IVe, IVf, IVg, IVh, IVi, IVj, IVk, IVl, IVm, IVn, and IVo. In some embodiments, the compound is a compound described herein, including a compound of any one of Formulae XI, XII, XIII, or XIV; a sub-formulae of any of the foregoing; an embodiment of any of the foregoing; or a compound described in a figure, table, aspect, embodiment, or claim herein. In some embodiments of the methods described herein, the method may include (e.g., use, implementation, or administration) any compound described herein, including a compound of any one of Formulae XI, XII, XIII, or XIV; a sub-formulae of any of the foregoing; or an embodiment of any of the foregoing; or a compound described in a figure, table, aspect, embodiment, or claim herein.

In some embodiments, the compound has the formula:

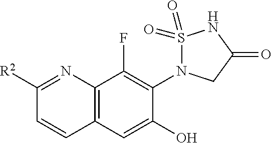

and R² is as described herein. In some embodiments, the compound has the formula:

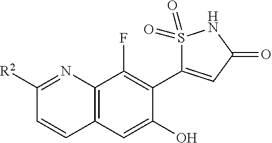

and R² is as described herein. In some embodiments, the compound has the formula:

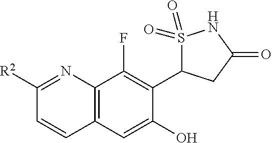

and R² is as described herein. In some embodiments, the compound has the formula:

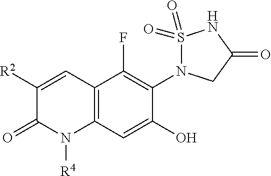

and R² and R⁴ are as described herein. In some embodiments, the compound has the formula:

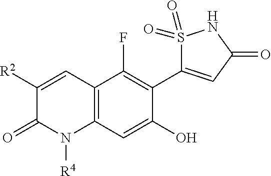

and R² and R⁴ are as described herein. In some embodiments, the compound has the formula:

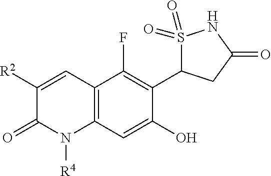

and R² and R⁴ are as described herein. In some embodiments, the compound has the formula:

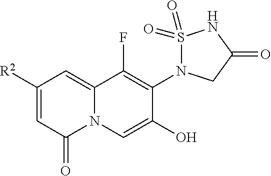

and R² is as described herein. In some embodiments, the compound has the formula:

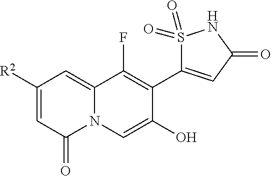

and R² is as described herein. In some embodiments, the compound has the formula:

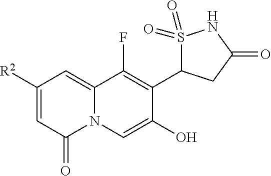

and R² is as described herein. In some embodiments, the compound has the formula:

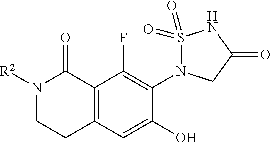

and R² is as described herein. In some embodiments, the compound has the formula:

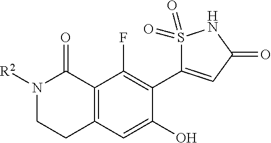

and R² is as described herein. In some embodiments, the compound has the formula:

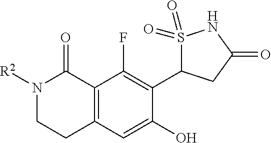

and R² is as described herein. In some embodiments, the compound has the formula:

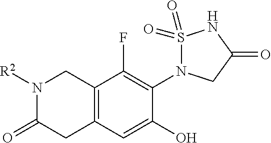

and R² is as described herein. In some embodiments, the compound has the formula:

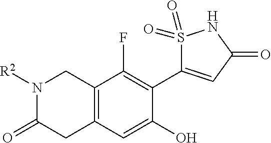

and R² is as described herein. In some embodiments, the compound has the formula:

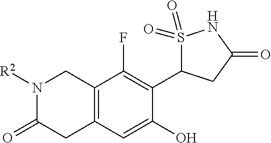

and R² is as described herein. In some embodiments, the compound has the formula:

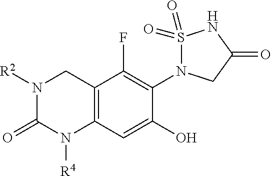

and R² is as described herein. In some embodiments, the compound has the formula:

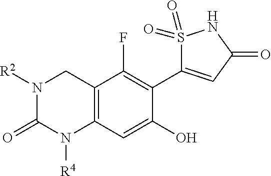

and R² is as described herein. In some embodiments, the compound has the formula:

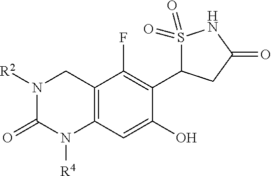

and R² is as described herein. In some embodiments, the compound has the formula:

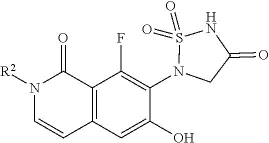

and R² is as described herein. In some embodiments, the compound has the formula:

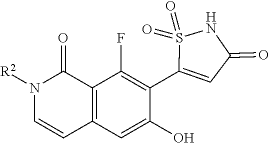

and R² is as described herein. In some embodiments, the compound has the formula:

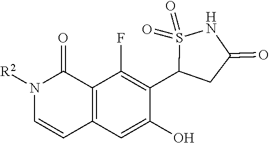

and R² is as described herein. In some embodiments, the compound has the formula:

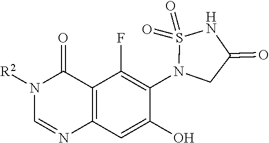

and R² is as described herein. In some embodiments, the compound has the formula:

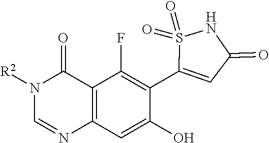

and R² is as described herein. In some embodiments, the compound has the formula:

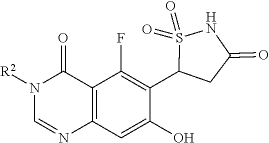

and R² is as described herein.

In embodiments, each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20a} is independently oxo. In some embodiments R^{20a} is independently —CN. In some embodiments R^{20a} is independently halogen. In some embodiments R^{20a} is independently —CN. In some embodiments R^{20a} is independently C₁₋₆ alkyl. In some embodiments R^{20a} is independently C₂₋₆alkenyl. In some embodiments R^{20a} is independently C₂₋₆alkynyl. In some embodiments R^{20a} is independently C₃₋₆cycloalkyl. In some embodiments R^{20a} is independently —CH₂—C₃₋₆cycloalkyl. In some embodiments R^{20a} is independently C₁₋₆heteroalkyl. In some embodiments R^{20a} is independently C₂₋₉heterocycloalkyl. In some embodiments R^{20a} is independently —CH₂—C₂₋₉heterocycloalkyl. In some embodiments R^{20a} is independently C₆₋₁₀aryl. In some embodiments R^{20a} is independently —CH₂—C₆₋₁₀aryl. In some embodiments R^{20a} is independently C₁₋₉heteroaryl. In some embodiments R^{20a} is independently —OR²¹. In some embodiments R^{20a} is independently —SR²¹. In some embodiments R^{20a} is independently —N(R²²)(R²³). In some embodiments R^{20a} is independently —C(O)OR²². In some embodiments R^{20a} is independently —C(O)N(R²²)(R²³). In some embodiments R^{20a} is independently —C(O)C(O)N(R²²)(R²³). In some embodiments R^{20a} is independently —OC(O)N(R²²)(R²³). In some embodiments R^{20a} is independently —N(R²⁴)C(O)N(R²²)(R²³). In some embodiments R^{20a} is independently —N(R²⁴)C(O)OR²². In some embodiments R^{20a} is independently —N(R²⁴)C(O)R²⁵. In some embodiments R^{20a} is independently —N(R²⁴)S(O)₂R²⁵. In some embodiments R^{20a} is independently —C(O)R²⁵. In some embodiments R^{20a} is independently —S(O)₂R²⁵. In some embodiments R^{20a} is independently —S(O)₂N(R²²)(R²³). In some embodiments R^{20a} is independently —OCH₂C(O)OR²². In some embodiments R^{20a} is independently —OC(O)R²⁵. In some embodiments R^{20a} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20a} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20a} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20a} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20a} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20a} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20a} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20a} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20a} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20a} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20a} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20a} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R²¹ is independently oxo. In some embodiments R^{20b} is independently —CN. In some embodiments R^{20b} is independently halogen. In some embodiments R²¹ is independently —CN. In some embodiments R²¹ is independently C₁₋₆ alkyl. In some embodiments R^{20b} is independently C₂₋₆alkenyl. In some embodiments R^{20b} is independently C₂₋₆alkynyl. In some embodiments R^{20b} is independently C₃₋₆cycloalkyl. In some embodiments R^{20b} is independently —CH₂—C₃₋₆cycloalkyl. In some embodiments R^{20b} is independently C₁₋₆heteroalkyl. In some embodiments R^{20b} is independently C₂₋₉heterocycloalkyl. In some embodiments R^{20b} is independently —CH₂—C₂₋₉heterocycloalkyl. In some embodiments R^{20b} is independently C₆₋₁₀aryl. In some embodiments R^{20b} is independently —CH₂—C₆₋₁₀aryl. In some embodiments R^{20b} is independently C₁₋₉heteroaryl. In some embodiments R^{20b} is independently —OR²¹. In some embodiments R^{20b} is independently —SR²¹. In some embodiments R^{20b} is independently —N(R²²)(R²³). In some embodiments R^{20b} is independently —C(O)OR²². In some embodiments R^{20b} is independently —C(O)N(R²²)(R²³). In some embodiments R²¹ is independently —C(O)C(O)N(R²²)(R²³). In some embodiments R²¹ is independently —OC(O)N(R²²)(R²³). In some embodiments R²¹ is independently —N(R¹)C(O)N(R²²)(R²³). In some embodiments R^{20b} is independently —N(R²⁴)C(O)OR²². In some embodiments R²¹ is independently —N(R¹)C(O)R²⁵. In some embodiments R^{20b} is independently —N(R²⁴)S(O)₂R²⁵. In some embodiments R^{20b} is independently —C(O)R²⁵. In some embodiments R^{20b} is independently —S(O)₂R²⁵. In some embodiments R^{20b} is independently —S(O)₂N(R²²)(R²³). In some embodiments R^{20b} is independently —OCH₂C(O)OR²². In some embodiments R^{20b} is independently —OC(O)R²⁵. In some embodiments R^{20b} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20b} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20b} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20b} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20b} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20b} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20b} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20b} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²¹ is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20b} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²¹ is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R²¹ is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20c} is independently oxo. In some embodiments R^{20c} is independently —CN. In some embodiments R^{20c} is independently halogen. In some embodiments R^{20c} is independently —CN. In some embodiments R^{20c} is independently C₁₋₆ alkyl. In some embodiments R^{20c} is independently C₂₋₆alkenyl. In some embodiments R^{20c} is independently C₂₋₆alkynyl. In some embodiments R^{2c} is independently C₃₋₆cycloalkyl. In some embodiments R^{2c} is independently —CH₂—C₃₋₆cycloalkyl. In some embodiments R^{2c} is independently C₁₋₆heteroalkyl. In some embodiments R^{2c} is independently C₂₋₉heterocycloalkyl. In some embodiments R^{2c} is independently —CH₂—C₂₋₉heterocycloalkyl. In some embodiments R² is independently C₆₋₁₀aryl. In some embodiments R^{20c} is independently —CH₂—C₆₋₁₀aryl. In some embodiments R^{20c} is independently C₁₋₉heteroaryl. In some embodiments R^{20c} is independently —OR²¹. In some embodiments R^{20c} is independently —SR²¹. In some embodiments R^{20c} is independently —N(R²²)(R²³). In some embodiments R^{20c} is independently —C(O)OR²². In some embodiments R^{20c} is independently —C(O)N(R²²)(R²³). In some embodiments R^{20c} is independently —C(O)C(O)N(R²²)(R²³). In some embodiments R^{20c} is independently —OC(O)N(R²²)(R²³). In some embodiments R² is independently —N(R²⁴)C(O)N(R²²)(R²³). In some embodiments R² is independently —N(R²⁴)C(O)OR²². In some embodiments R² is independently —N(R²⁴)C(O)R²⁵. In some embodiments R² is independently —N(R²⁴)S(O)₂R²⁵. In some embodiments R^{2c} is independently —C(O)R²⁵. In some embodiments R^{2c} is independently —S(O)₂R²⁵. In some embodiments R^{20c} is independently —S(O)₂N(R²²)(R²³). In some embodiments R^{2c} is independently —OCH₂C(O)OR²². In some embodiments R² is independently —OC(O)R²⁵. In some embodiments R^{20c} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20c} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²¹ is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20c} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²⁰⁰ is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20c} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20c} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20c} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20c} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20c} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20c} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R²¹ is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R² is independently oxo. In some embodiments R^{20d} is independently —CN. In some embodiments R^{20d} is independently halogen. In some embodiments R^{20d} is independently —CN. In some embodiments R^{20d} is independently C₁₋₆ alkyl. In some embodiments R^{20d} is independently C₂₋₆alkenyl. In some embodiments R^{20d} is independently C₂₋₆alkynyl. In some embodiments R^{20d} is independently C₃₋₆cycloalkyl. In some embodiments R^{20d} is independently —CH₂—C₃₋₆cycloalkyl. In some embodiments R^{20d} is independently C₁₋₆heteroalkyl. In some embodiments R^{20d} is independently C₂₋₉heterocycloalkyl. In some embodiments R^{20d} is independently —CH₂—C₂₋₉heterocycloalkyl. In some embodiments R^{20d} is independently C₆₋₁₀aryl. In some embodiments R^{20d} is independently —CH₂—C₆₋₁₀aryl. In some embodiments R²¹ is independently C₁₋₉heteroaryl. In some embodiments R^{20d} is independently —OR²¹. In some embodiments R²¹ is independently —SR²¹. In some embodiments R^{20d} is independently —N(R²²)(R²³). In some embodiments R^{20d} is independently —C(O)OR²². In some embodiments R^{20d} is independently —C(O)N(R²²)(R²³). In some embodiments R²¹ is independently —C(O)C(O)N(R²²)(R²³). In some embodiments R²¹ is independently —OC(O)N(R²²)(R²³). In some embodiments R²¹ is independently —N(R²⁴)C(O)N(R²²)(R²³). In some embodiments R^{20d} is independently —N(R²⁴)C(O)OR²². In some embodiments R^{20d} is independently —N(Ru)C(O)R²⁵. In some embodiments R^{20d} is independently —N(R²⁴)S(O)₂R²⁵. In some embodiments R^{20d} is independently —C(O)R²⁵. In some embodiments R^{20d} is independently —S(O)₂R²⁵. In some embodiments R^{20d} is independently —S(O)₂N(R²²)(R²³). In some embodiments R^{20d} is independently —OCH₂C(O)OR²². In some embodiments R^{20d} is independently —OC(O)R²⁵. In some embodiments R^{20d} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R² is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20d} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20d} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20d} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20d} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20d} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20d} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20d} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20d} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R² is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20d} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵.

In some embodiments R^{20e} is independently oxo. In some embodiments R^{20e} is independently —CN. In some embodiments R^{20e} is independently halogen. In some embodiments R^{20e} is independently —CN. In some embodiments R^{20e} is independently C₁₋₆ alkyl. In some embodiments R^{20e} is independently C₂₋₆alkenyl. In some embodiments R^{20e} is independently C₂₋₆alkynyl. In some embodiments R^{20e} is independently C₃₋₆cycloalkyl. In some embodiments R^{20e} is independently —CH₂—C₃₋₆cycloalkyl. In some embodiments R^{20e} is independently C₁₋₆heteroalkyl. In some embodiments R^{20e} is independently C₂₋₉heterocycloalkyl. In some embodiments R^{20e} is independently —CH₂—C₂₋₉heterocycloalkyl. In some embodiments R^{20e} is independently C₆₋₁₀aryl. In some embodiments R^{20e} is independently —CH₂—C₆₋₁₀aryl. In some embodiments R^{20e} is independently C₁₋₉heteroaryl. In some embodiments R^{20e} is independently —OR²¹. In some embodiments R^{20e} is independently —SR²¹. In some embodiments R^{20e} is independently —N(R²²)(R²³). In some embodiments R^{20e} is independently —C(O)OR²². In some embodiments R^{20e} is independently —C(O)N(R²²)(R²³). In some embodiments R^{20e} is independently —C(O)C(O)N(R²²)(R²³). In some embodiments R^{20e} is independently —OC(O)N(R²²)(R²³). In some embodiments R^{20e} is independently —N(R²⁴)C(O)N(R²²)(R²³). In some embodiments R^{20e} is independently —N(R²⁴)C(O)OR²². In some embodiments R^{20e} is independently —N(R²⁴)C(O)R²⁵. In some embodiments R^{20e} is independently —N(R²⁴)S(O)₂R²⁵. In some embodiments R^{20e} is independently —C(O)R²⁵. In some embodiments R^{20e} is independently —S(O)₂R²⁵. In some embodiments R^{20e} is independently —S(O)₂N(R²²)(R²³). In some embodiments R^{20e} is independently —OCH₂C(O)OR²². In some embodiments R^{20e} is independently —OC(O)R²⁵. In some embodiments R^{20e} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20e} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20e} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20e} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20e} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20e} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20e} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20e} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20e} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20e} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20e} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20e} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20f} is independently oxo. In some embodiments R^{20f} is independently —CN. In some embodiments R^{20f} is independently halogen. In some embodiments R^{20f} is independently —CN. In some embodiments R^{20f} is independently C₁₋₆ alkyl. In some embodiments R^{20f} is independently C₂₋₆alkenyl. In some embodiments R^{20f} is independently C₂₋₆alkynyl. In some embodiments R^{20f} is independently C₃₋₆cycloalkyl. In some embodiments R^{20f} is independently —CH₂—C₃₋₆cycloalkyl. In some embodiments R^{20f} is independently C₁₋₆heteroalkyl. In some embodiments R^{20f} is independently C₂₋₉heterocycloalkyl. In some embodiments R^{20f} is independently —CH₂—C₂₋₉heterocycloalkyl. In some embodiments R^{20f} is independently C₆₋₁₀aryl. In some embodiments R^{20f} is independently —CH₂—C₆₋₁₀aryl. In some embodiments R^{20f} is independently C₁₋₉heteroaryl. In some embodiments R^{20f} is independently —OR²¹. In some embodiments R²⁰ is independently —SR²¹. In some embodiments R²⁰ is independently —N(R²²)(R²³). In some embodiments R²⁰ is independently —C(O)OR²². In some embodiments R²⁰ is independently —C(O)N(R²²)(R²³). In some embodiments R²⁰ is independently —C(O)C(O)N(R²²)(R²³). In some embodiments R²⁰ is independently —OC(O)N(R²²)(R²³). In some embodiments R^{20f} is independently —N(R²⁴)C(O)N(R²²)(R²³). In some embodiments R^{20f} is independently —N(R²⁴)C(O)OR²². In some embodiments R^{20f} is independently —N(R²⁴)C(O)R²⁵. In some embodiments R^{20f} is independently —N(R²⁴)S(O)₂R²⁵. In some embodiments R^{20f} is independently —C(O)R²⁵. In some embodiments R^{20f} is independently —S(O)₂R²⁵. In some embodiments R^{20f} is independently —S(O)₂N(R²²)(R²³). In some embodiments R^{20f} is independently —OCH₂C(O)OR²². In some embodiments R^{20f} is independently —OC(O)R²⁵. In some embodiments R^{20f} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20f} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20f} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20f} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20f} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20f} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20f} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20f} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R^{20f} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20g} is independently oxo. In some embodiments R^{20g} is independently —CN. In some embodiments R^{20g} is independently halogen. In some embodiments R^{20g} is independently —CN. In some embodiments R^{20g} is independently C₁₋₆ alkyl. In some embodiments R^{20g} is independently C₂₋₆alkenyl. In some embodiments R^{20g} is independently C₂₋₆alkynyl. In some embodiments R^{20g} is independently C₃₋₆cycloalkyl. In some embodiments R^{20g} is independently —CH₂—C₃₋₆cycloalkyl. In some embodiments R^{20g} is independently C₁₋₆heteroalkyl. In some embodiments R^{20g} is independently C₂₋₉heterocycloalkyl. In some embodiments R^{20g} is independently —CH₂—C₂₋₉heterocycloalkyl. In some embodiments R^{20g} is independently C₆₋₁₀aryl. In some embodiments R^{20g} is independently —CH₂—C₆₋₁₀aryl. In some embodiments R^{20g} is independently C₁₋₉heteroaryl. In some embodiments R^{20g} is independently —OR²¹. In some embodiments R^{20g} is independently —SR²¹. In some embodiments R^{20g} is independently —N(R²²)(R²³). In some embodiments R^{20g} is independently —C(O)OR²². In some embodiments R^{20g} is independently —C(O)N(R²²)(R²³). In some embodiments R^{20g} is independently —C(O)C(O)N(R²²)(R²³). In some embodiments R^{20g} is independently —OC(O)N(R²²)(R²³). In some embodiments R^{20g} is independently —N(R⁴)C(O)N(R²²)(R²³). In some embodiments R^{20g} is independently —N(R²⁴)C(O)OR²². In some embodiments R^{20g} is independently —N(R²⁴)C(O)R²⁵. In some embodiments R^{20g} is independently —N(R²⁴)S(O)₂R²⁵. In some embodiments R^{20g} is independently —C(O)R²⁵. In some embodiments R^{20g} is independently —S(O)₂R²⁵. In some embodiments R^{20g} is independently —S(O)₂N(R²²)(R²³). In some embodiments R^{20g} is independently —OCH₂C(O)OR²². In some embodiments R^{20g} is independently —OC(O)R²⁵. In some embodiments R^{20g} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20g} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20g} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20g} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20g} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20g} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20g} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20h} is independently oxo. In some embodiments R^{20h} is independently —CN. In some embodiments R^{20h} is independently halogen. In some embodiments R^{20h} is independently —CN. In some embodiments R^{20h} is independently C₁₋₆ alkyl. In some embodiments R^{20h} is independently C₂₋₆alkenyl. In some embodiments R^{20h} is independently C₂₋₆alkynyl. In some embodiments R^{20h} is independently C₃₋₆cycloalkyl. In some embodiments R^{20h} is independently —CH₂—C₃₋₆cycloalkyl. In some embodiments R^{20h} is independently C₁₋₆heteroalkyl. In some embodiments R^{20h} is independently C₂₋₉heterocycloalkyl. In some embodiments R^{20h} is independently —CH₂—C₂₋₉heterocycloalkyl. In some embodiments R^{20h} is independently C₆₋₁₀aryl. In some embodiments R^{20h} is independently —CH₂—C₆₋₁₀aryl. In some embodiments R^{20h} is independently C₁₋₉heteroaryl. In some embodiments R^{20h} is independently —OR²¹. In some embodiments R^{20h} is independently —SR²¹. In some embodiments R^{20h} is independently —N(R²²)(R²³). In some embodiments R^{20h} is independently —C(O)OR²². In some embodiments R^{20h} is independently —C(O)N(R²²)(R²³). In some embodiments R^{20h} is independently —C(O)C(O)N(R²²)(R²³). In some embodiments R^{20h} is independently —OC(O)N(R²²)(R²³). In some embodiments R^{20h} is independently —N(R¹)C(O)N(R²²)(R²³). In some embodiments R^{20h} is independently —N(R²⁴)C(O)OR²². In some embodiments R^{20h} is independently —N(R²⁴)C(O)R²⁵. In some embodiments R^{20h} is independently —N(R²⁴)S(O)₂R²⁵. In some embodiments R^{20h} is independently —C(O)R²⁵. In some embodiments R^{20h} is independently —S(O)₂R²⁵. In some embodiments R^{20h} is independently —S(O)₂N(R²²)(R²³). In some embodiments R^{20h} is independently —OCH₂C(O)OR²². In some embodiments R^{20h} is independently —OC(O)R²⁵. In some embodiments R^{20h} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20h} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20h} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20h} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20h} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20h} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20h} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20h} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20h} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20h} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20h} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20h} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵.

In some embodiments R²⁰ⁱ is independently oxo. In some embodiments R²⁰ⁱ is independently —CN. In some embodiments R²⁰ⁱ is independently halogen. In some embodiments R²⁰ⁱ is independently —CN. In some embodiments R²⁰ⁱ is independently C₁₋₆ alkyl. In some embodiments R²¹ is independently C₂₋₆alkenyl. In some embodiments R²⁰ⁱ is independently C₂₋₆alkynyl. In some embodiments R²⁰ⁱ is independently C₃₋₆cycloalkyl. In some embodiments R²⁰ⁱ is independently —CH₂—C₃₋₆cycloalkyl. In some embodiments R²⁰ⁱ is independently C₁₋₆heteroalkyl. In some embodiments R²⁰ⁱ is independently C₂₋₉heterocycloalkyl. In some embodiments R²⁰ⁱ is independently —CH₂—C₂₋₉heterocycloalkyl. In some embodiments R²⁰ⁱ is independently C₆₋₁₀aryl. In some embodiments R²⁰ⁱ is independently —CH₂—C₆₋₁₀aryl. In some embodiments R²⁰ is independently C₁₋₉heteroaryl. In some embodiments R²⁰ⁱ is independently —OR²¹. In some embodiments R²¹ is independently —SR²¹. In some embodiments R²⁰ⁱ is independently —N(R²²)(R²³). In some embodiments R²⁰ⁱ is independently —C(O)OR²². In some embodiments R²⁰ⁱ is independently —C(O)N(R²²)(R²³). In some embodiments R²¹ is independently —C(O)C(O)N(R²²)(R²³). In some embodiments R²⁰ⁱ is independently —OC(O)N(R²²)(R²³). In some embodiments R²¹ is independently —N(R²⁴)C(O)N(R²²)(R²³). In some embodiments R²⁰ⁱ is independently —N(R²⁴)C(O)OR²². In some embodiments R²⁰ is independently —N(R²⁴)C(O)R²⁵. In some embodiments R²⁰ⁱ is independently —N(R²⁴)S(O)₂R²⁵. In some embodiments R²⁰ⁱ is independently —C(O)R²⁵. In some embodiments R²⁰ⁱ is independently —S(O)₂R²⁵. In some embodiments R²⁰ⁱ is independently —S(O)₂N(R²²)(R²³). In some embodiments R²⁰ⁱ is independently —OCH₂C(O)OR²². In some embodiments R²⁰ⁱ is independently —OC(O)R²⁵. In some embodiments R²⁰ⁱ is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R²⁰ⁱ is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R² is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R² is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R² is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R² is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R²⁰ⁱ is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20j} is independently oxo. In some embodiments R^{20j} is independently —CN. In some embodiments R^{20j} is independently halogen. In some embodiments R^{20j} is independently —CN. In some embodiments R^{20j} is independently C₁₋₆ alkyl. In some embodiments R²⁰ⁱ is independently C₂₋₆alkenyl. In some embodiments R²⁰ⁱ is independently C₂₋₆alkynyl. In some embodiments R^{20j} is independently C₃₋₆cycloalkyl. In some embodiments R²⁰ⁱ is independently —CH₂—C₃₋₆cycloalkyl. In some embodiments R^{20j} is independently C₁₋₆heteroalkyl. In some embodiments R^{20j} is independently C₂₋₉heterocycloalkyl. In some embodiments R^{20j} is independently —CH₂—C₂₋₉heterocycloalkyl. In some embodiments R^{20j} is independently C₆₋₁₀aryl. In some embodiments R^{20j} is independently —CH₂—C₆₋₁₀aryl. In some embodiments R^{20j} is independently C₁₋₉heteroaryl. In some embodiments R^{20j} is independently —OR²¹. In some embodiments R^{20j} is independently —SR²¹. In some embodiments R^{20j} is independently —N(R²²)(R²³). In some embodiments R^{20j} is independently —C(O)OR²². In some embodiments R^{20j} is independently —C(O)N(R²²)(R²³). In some embodiments R^{20j} is independently —C(O)C(O)N(R²²)(R²³). In some embodiments R^{20j} is independently —OC(O)N(R²²)(R²³). In some embodiments R²⁰ⁱ is independently —N(R²⁴)C(O)N(R²²)(R²³). In some embodiments R^{20j} is independently —N(R²⁴)C(O)OR²². In some embodiments R²⁰ⁱ is independently —N(R²⁴)C(O)R²⁵. In some embodiments R^{20j} is independently —N(R²⁴)S(O)₂R²⁵. In some embodiments R^{20j} is independently —C(O)R²⁵. In some embodiments R^{20j} is independently —S(O)₂R²⁵. In some embodiments R^{20j} is independently —S(O)₂N(R²²)(R²³). In some embodiments R^{20j} is independently —OCH₂C(O)OR²². In some embodiments R^{20j} is independently —OC(O)R²⁵. In some embodiments R^{20j} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20j} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R²⁰ⁱ is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20g} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20j} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²⁰ⁱ is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20j} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R²⁰ⁱ is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20j} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵.

In some embodiments R^{20k} is independently oxo. In some embodiments R^{20k} is independently —CN. In some embodiments R^{20k} is independently halogen. In some embodiments R^{20k} is independently —CN. In some embodiments R^{20k} is independently C₁₋₆ alkyl. In some embodiments R^{20k} is independently C₂₋₆alkenyl. In some embodiments R^{20k} is independently C₂₋₆alkynyl. In some embodiments R^{20k} is independently C₃₋₆cycloalkyl. In some embodiments R^{20k} is independently —CH₂—C₃₋₆cycloalkyl. In some embodiments R^{20k} is independently C₁₋₆heteroalkyl. In some embodiments R^{20k} is independently C₂₋₉heterocycloalkyl. In some embodiments R^{20k} is independently —CH₂—C₂₋₉heterocycloalkyl. In some embodiments R^{20k} is independently C₆₋₁₀aryl. In some embodiments R^{20k} is independently —CH₂—C₆₋₁₀aryl. In some embodiments R^{20k} is independently C₁₋₉heteroaryl. In some embodiments R^{20k} is independently —OR²¹. In some embodiments R^{20k} is independently —SR²¹. In some embodiments R^{20k} is independently —N(R²²)(R²³). In some embodiments R^{20k} is independently —C(O)OR²². In some embodiments R^{20k} is independently —C(O)N(R²²)(R²³). In some embodiments R^{20k} is independently —C(O)C(O)N(R²²)(R²³). In some embodiments R^{20k} is independently —OC(O)N(R²²)(R²³). In some embodiments R^{20k} is independently —N(R²⁴)C(O)N(R²²)(R²³). In some embodiments R^{20k} is independently —N(R²⁴)C(O)OR²². In some embodiments R^{20k} is independently —N(R²⁴)C(O)R²⁵. In some embodiments R^{20k} is independently —N(R²⁴)S(O)₂R²⁵. In some embodiments R^{20k} is independently —C(O)R²⁵. In some embodiments R^{20k} is independently —S(O)₂R²⁵. In some embodiments R^{20k} is independently —S(O)₂N(R²²)(R²³). In some embodiments R^{20k} is independently —OCH₂C(O)OR²². In some embodiments R^{20k} is independently —OC(O)R²⁵. In some embodiments R^{20k} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20k} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20k} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20k} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20k} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20k} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20k} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20k} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20k} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20k} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20k} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵.

In some embodiments R^{20l} is independently oxo. In some embodiments R^{20l} is independently —CN. In some embodiments R^{20l} is independently halogen. In some embodiments R^{20l} is independently —CN. In some embodiments R^{20l} is independently C₁₋₆ alkyl. In some embodiments R^{20l} is independently C₂₋₆alkenyl. In some embodiments R^{20l} is independently C₂₋₆alkynyl. In some embodiments R^{20l} is independently C₃₋₆cycloalkyl. In some embodiments R^{20l} is independently —CH₂—C₃₋₆cycloalkyl. In some embodiments R^{20l} is independently C₁₋₆heteroalkyl. In some embodiments R^{20l} is independently C₂₋₉heterocycloalkyl. In some embodiments R^{20l} is independently —CH₂—C₂₋₉heterocycloalkyl. In some embodiments R^{20l} is independently C₆₋₁₀aryl. In some embodiments R^{20l} is independently —CH₂—C₆₋₁₀aryl. In some embodiments R^{20l} is independently C₁₋₉heteroaryl. In some embodiments R^{20l} is independently —OR²¹. In some embodiments R^{20l} is independently —SR²¹. In some embodiments R^{20l} is independently —N(R²²)(R²³). In some embodiments R^{20l} is independently —C(O)OR²². In some embodiments R^{20l} is independently —C(O)N(R²²)(R²³). In some embodiments R^{20l} is independently —C(O)C(O)N(R²²)(R²³). In some embodiments R^{20l} is independently —OC(O)N(R²²)(R²³). In some embodiments R^{20l} is independently —N(R¹)C(O)N(R²²)(R²³). In some embodiments R^{20l} is independently —N(R²⁴)C(O)OR²². In some embodiments R^{20l} is independently —N(R′)C(O)R²⁵. In some embodiments R^{20l} is independently —N(R²⁴)S(O)₂R²⁵. In some embodiments R^{20l} is independently —C(O)R²⁵. In some embodiments R^{20l} is independently —S(O)₂R²⁵. In some embodiments R^{20l} is independently —S(O)₂N(R²²)(R²³). In some embodiments R^{20l} is independently —OCH₂C(O)OR²². In some embodiments R^{20l} is independently —OC(O)R²⁵. In some embodiments R^{20l} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20l} is independently C₂₋₆alkenyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20l} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20l} is independently C₃₋₆cycloalkyl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20l} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20l} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20l} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20l} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20l} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20l} is independently —CH₂—C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments R^{20l} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R^{20l} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments R^{20m} is independently oxo. In some embodiments R² is independently —CN. In some embodiments R^{20m} is independently halogen. In some embodiments R^{20m} is independently —CN. In some embodiments R^{20m} is independently C₁₋₆ alkyl. In some embodiments R^{20m} is independently C₂₋₆alkenyl. In some embodiments R^{20m} is independently C₂₋₆alkynyl. In some embodiments R^{20m} is independently C₃₋₆cycloalkyl. In some embodiments R^{20m} is independently —CH₂—C₃₋₆cycloalkyl. In some embodiments R^{20m} is independently C₁₋₆heteroalkyl. In some embodiments R^{20m} is independently C₂₋₉heterocycloalkyl. In some embodiments R^{20m} is independently —CH₂—C₂₋₉heterocycloalkyl. In some embodiments R^{20m} is independently C₆₋₁₀aryl. In some embodiments R^{20m} is independently —CH₂—C₆₋₁₀aryl. In some embodiments R² is independently C₁₋₉heteroaryl. In some embodiments R^{20m} is independently —OR²¹. In some embodiments R² is independently —SR²¹. In some embodiments R² is independently —N(R²²)(R²³). In some embodiments R^{20m} is independently —C(O)OR²². In some embodiments R^{20m} is independently —C(O)N(R²²)(R²³). In some embodiments R^{20m} is independently —C(O)C(O)N(R²²)(R²³). In some embodiments R^{20m} is independently —OC(O)N(R²²)(R²³). In some embodiments R^{20m} is independently —N(R²⁴)C(O)N(R²²)(R²³). In some embodiments R^{20m} is independently —N(R²⁴)C(O)OR²². In some embodiments R^{20m} is independently —N(R²⁴)C(O)R²⁵. In some embodiments R^{20m} is independently —N(Ru)S(O)₂R²⁵. In some embodiments R^{20m} is independently —C(O)R²⁵. In some embodiments R^{20m} is independently —S(O)₂R²⁵. In some embodiments R^{20m} is independently —S(O)₂N(R²²)(R²³). In some embodiments R² is independently —OCH₂C(O)OR²². In some embodiments R² is independently —OC(O)R²⁵. In some embodiments R^{20m} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20m} is independently C₂₋₆alkenyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20m} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20m} is independently C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20m} is independently —CH₂—C₃₋₆cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20m} is independently C₁₋₆heteroalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20m} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{20m} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20m} is independently C₆₋₁₀aryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments R^{2m} is independently —CH₂—C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments R^{20m} is independently C₁₋₉heteroaryl substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, R^{20m} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R²¹ is independently H. In some embodiments, R²¹ is independently C₁₋₆alkyl. In some embodiments, R²¹ is independently C₁₋₆haloalkyl. In some embodiments, R²¹ is independently C₂₋₆alkenyl. In some embodiments, R²¹ is independently C₂₋₆alkynyl. In some embodiments, R²¹ is independently C₃₋₆cycloalkyl. In some embodiments, R²¹ is independently C₂₋₉heterocycloalkyl. In some embodiments, R²¹ is independently C₆₋₁₀aryl. In some embodiments, R²¹ is independently C₁₋₉ heteroaryl.

In some embodiments, R²² is independently H. In some embodiments, R²² is independently C₁₋₆alkyl. In some embodiments, R²² is independently C₁₋₆haloalkyl. In some embodiments, R²² is independently C₂₋₆alkenyl. In some embodiments, R²² is independently C₂₋₆alkynyl. In some embodiments, R²² is independently C₃₋₆cycloalkyl. In some embodiments, R²² is independently C₂₋₉heterocycloalkyl. In some embodiments, R²² is independently C₆₋₁₀aryl. In some embodiments, R²² is independently C₁₋₉ heteroaryl.

In some embodiments, R²³ is independently H. In some embodiments, R²³ is independently C₁₋₆alkyl.

In some embodiments, R²⁴ is independently H. In some embodiments, R²⁴ is independently C₁₋₆alkyl.

In some embodiments, R²⁵ is independently C₁₋₆alkyl. In some embodiments, R²⁵ is independently C₂₋₆alkenyl. In some embodiments, R²⁵ is independently C₂₋₆alkynyl. In some embodiments, R²⁵ is independently C₃₋₆cycloalkyl. In some embodiments, R²⁵ is independently C₁₋₆heteroalkyl. In some embodiments, R²⁵ is independently C₂₋₉heterocycloalkyl. In some embodiments, R²⁵ is independently C₆₋₁₀aryl. In some embodiments, R²⁵ is independently C₁₋₉heteroaryl. In some embodiments, R²⁵ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl.

In an aspect, provided is a compound, or a pharmaceutically acceptable salt or solvate thereof, selected from:

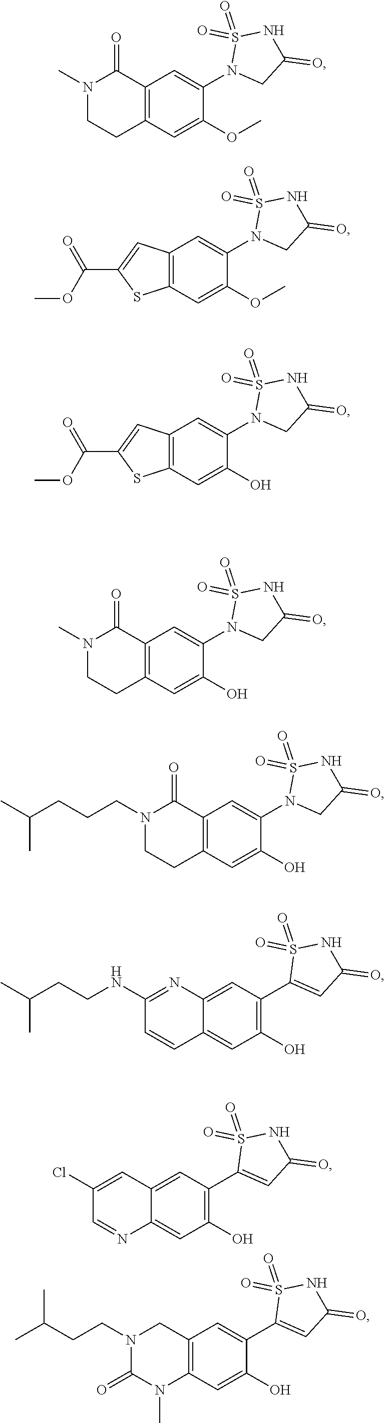

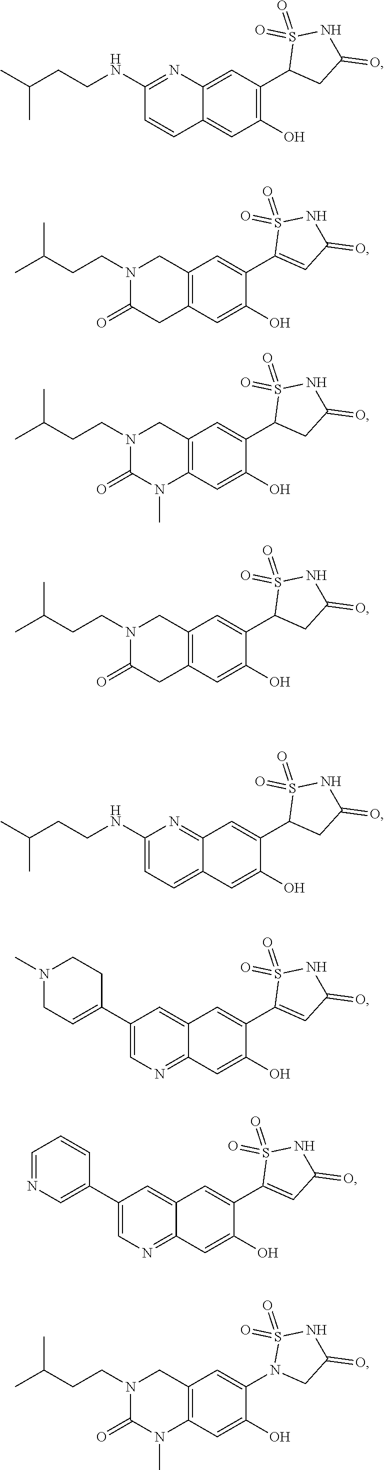

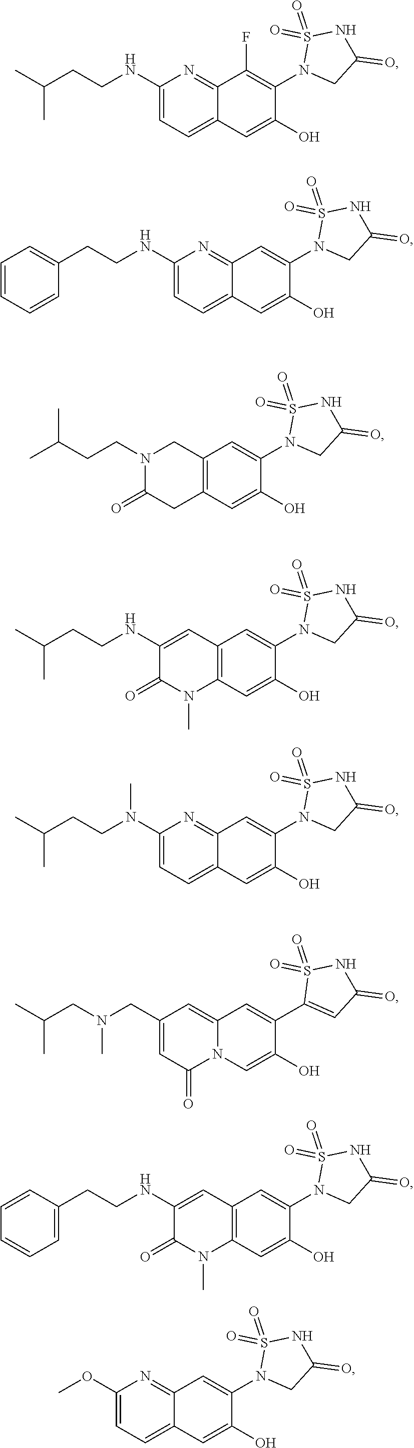

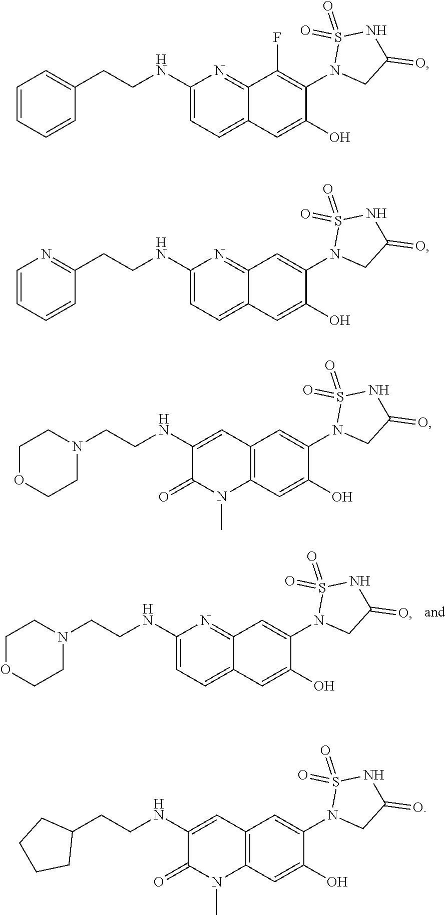

In some embodiments, the compound is a compound described herein, including a compound of any one of Formulae I, II, III, or IV; a sub-formulae of any of the foregoing; an embodiment of any of the foregoing; or a compound described in a figure, table, aspect, embodiment, or claim herein. In some embodiments of the methods described herein, the method may include (e.g., use, implementation, or administration) any compound described herein, including a compound of any one of Formulae I, II, III, or IV; a sub-formulae of any of the foregoing; or an embodiment of any of the foregoing; or a compound described in a figure, table, aspect, embodiment, or claim herein. In some embodiments of the methods described herein, the method may include (e.g., use, implementation, or administration) any compound described herein, including a compound of any one of Formulae XI, XII, XIII, or XIV; a sub-formulae of any of the foregoing; or an embodiment of any of the foregoing; or a compound described in a figure, table, aspect, embodiment, or claim herein.

In as aspect is provided a compound of Formula (XI-1), or a pharmaceutically acceptable salt or solvate thereof:

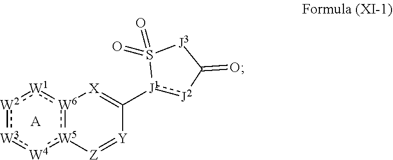

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N, C, or CH;
W⁶ is N, C, or CH;
wherein at least one of W¹, W², W³, W⁴, W⁵, and W⁶ is N, N(R¹), N(R²), N(R³), N(R⁴), or O; and
X is N or C(R⁵);
Y is N or C(R⁶);
Z is N or C(R⁷);
J¹ is N, C, or C(R⁷);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted;
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted;
R^{12a} is selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted;
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted;
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₂₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉ heteroaryl are optionally substituted;
[symbol] indicates a single or double bond such that all valences are satisfied; and provided that:
vii) at least two of Ring A [symbol] double bonds;
viii) when J³ is CH₂; R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; and
ix) the compound is not

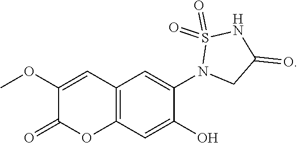 [SMILES: COc1cc2cc(N3CC(=O)NS3(=O)=O)c(O)cc2oc1=O]

In an aspect, provided is a compound of Formula (XIII-1), or a pharmaceutically acceptable salt or solvate thereof:

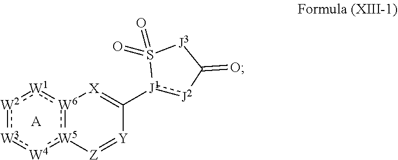

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N, C, or CH;
W⁶ is N, C, or CH; wherein
   v) at least one of W¹, W², W³, W⁴, W⁵, and W⁶ is N, N(R¹), N(R²), N(R³), N(R⁴), or O; and
   vi) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted; R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted;
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted;
each R¹⁶ is independently selected from —C₁₋₆alkylene-OP(O)(OR^{16a})(OR⁶b) and —P(O)(OR^{16a})(OR^{16b}); wherein the C₁₋₆alkylene is optionally substituted;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted;
[symbol] indicates a single or double bond such that all valences are satisfied; and
   provided the compound is not

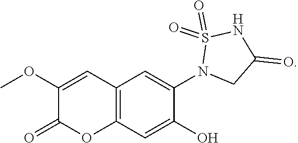 [SMILES: COc1cc2cc(N3CC(=O)NS3(=O)=O)c(O)cc2oc1=O]

In an aspect, provided is a compound of Formula (XII-1), or a pharmaceutically acceptable salt or solvate thereof:

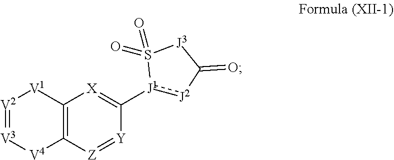

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R³ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted;
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted;
each R¹⁶ is independently selected from —C₁₋₆alkylene-OP(O)(OR^{16a})(OR⁶Ib) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted;

In an aspect, provided is a compound of Formula (XIV-1), or a pharmaceutically acceptable salt or solvate thereof:

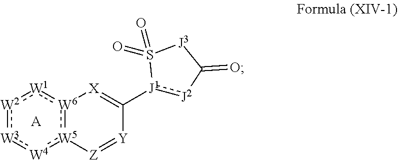

wherein:
W¹ is N, N(R¹), O, C(R¹), C(R¹)(R^{1a}), S(O), S(O)₂, S(O)(NR¹), S(O)(R¹), or P(O)(R¹);
W² is N, N(R²), O, C(R²), C(R²)(R^{2a}), S(O), S(O)₂, S(O)(NR²), S(O)(R²), or P(O)(R²);
W³ is N, N(R³), O, C(R³), C(R³)(R^{3a}), S(O), S(O)₂, S(O)(NR³), S(O)(R³), or P(O)(R³);
W⁴ is N, N(R⁴), O, C(R⁴), C(R⁴)(R^{4a}), S(O), S(O)₂, S(O)(NR⁴), S(O)(R⁴), or P(O)(R⁴);
W⁵ is N, C, or CH;
W⁶ is N, C, or CH; wherein at least one of W¹, W², W³, and W⁴ is S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴).
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R¹ and R^{1a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R² and R^{2a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R² and R^{2a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R³ and R^{3a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R³ and R^{3a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁴ and R^{4a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R⁴ and R^{4a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted;
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted; or R⁹ and R^{9a} are combined to form a C₃₋₁₀cycloalkyl or a C₂₋₉heterocycloalkyl, wherein the C₃₋₁₀cycloalkyl and C₂₋₉heterocycloalkyl are optionally substituted;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁_.heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted; or R¹⁰ and R^{10a} are combined to form a C₃₋₁₀cycloalkyl or a C₂₋₉heterocycloalkyl, wherein the C₃₋₁₀cycloalkyl and C₂₋₉heterocycloalkyl are optionally substituted;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted;
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted;
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted;
[symbol] indicates a single or double bond such that all valences are satisfied.

In some embodiments, the compound is a compound of Formula (Ia′-2):

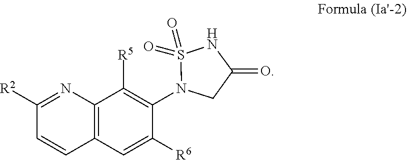

In some embodiments, the compound is a compound of Formula (Ia′-2), R⁵ is —F, R⁶ is —OH, and R² is C₁₋₆alkyl, optionally substituted with one, two, or three R^{20b}. In some embodiments, the compound is a compound of Formula (Ia′-2), R⁵ is —F, R⁶ is —OH, and R² is C₁₋₅alkyl, optionally substituted with one, two, or three R^{20b}. In some embodiments, the compound is a compound of Formula (Ia′-2), R⁵ is —F, R⁶ is —OH, and R² is C₁₋₄alkyl, optionally substituted with one, two, or three R^{20b}. In some embodiments, the compound is a compound of Formula (Ia′-2), R⁵ is —F, R⁶ is —OH, and R² is C₁₋₃alkyl, optionally substituted with one, two, or three R^{20b}. In some embodiments, the compound is a compound of Formula (Ia′-2), R⁵ is —F, R⁶ is —OH, and R² is C₁₋₂alkyl, optionally substituted with one, two, or three R^{20b}. In some embodiments, the compound is a compound of Formula (Ia′-2), R⁵ is —F, R⁶ is —OH, and R² is C₁alkyl, optionally substituted with one, two, or three R^{20b}. In some embodiments, the compound is a compound of Formula (Ia′-2), R⁵ is —F, R⁶ is —OH, and R² is C₂alkyl, optionally substituted with one, two, or three R^{20b}. In some embodiments, the compound is a compound of Formula (Ia′-2), R⁵ is —F, R⁶ is —OH, and R² is C₃alkyl, optionally substituted with one, two, or three R^{20b}In some embodiments, the compound is a compound of Formula (Ia′-2), R⁵ is —F, R⁶ is —OH, and R² is C₄alkyl, optionally substituted with one, two, or three R^{20b}. In some embodiments, the compound is a compound of Formula (Ia′-2), R⁵ is —F, R⁶ is —OH, and R² is C₅alkyl, optionally substituted with one, two, or three R^{20b}. In some embodiments, the compound is a compound of Formula (Ia′-2), R⁵ is —F, R⁶ is —OH, and R² is C₆alkyl, optionally substituted with one, two, or three R^{20b}. In some embodiments, the compound is a compound of Formula (Ia′-2), R⁵ is —F, R⁶ is —OH, and each R^{20b} is independently selected from —N(R²²)(R²³), R²² is C₁₋₆alkyl, and R²³ is C₁₋₆alkyl. In some embodiments, the compound is a compound of Formula (Ia′-2), R⁵ is —F, R⁶ is —OH, and R² is C₂₋₆alkynyl, optionally substituted with one, two, or three R^{20b}. In some embodiments, the compound is a compound of Formula (Ia′-2), R⁵ is —F, R⁶ is —OH, and R² is selected from any group listed in Table 2

In some embodiments, the compound is a compound of Formula (Ia′-2), R⁵ is —F, R⁶ is —OH, and R² is —OR¹². In some embodiments, the compound is a compound of Formula (Ia′-2), R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}. In some embodiments, the compound is a compound of Formula (Ia′-2), R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, the compound is a compound of Formula (Ia′-2), R⁵ is —F, R⁶ is —OH, and R² is —OR¹² and R¹² is independently selected from C₁₋₆alkyl, C₃₄cycloalkyl, C₃₋₅heterocycloalkyl, and —CH₂—C₄₋₅heterocycloalkyl, wherein C₁₋₆alkyl, C₃₄cycloalkyl, C₃₋₅heterocycloalkyl, and —CH₂—C₄₋₅ heterocycloalkyl, are optionally substituted with one, two, or three R^{20k}; and each R^{20k} is independently selected from oxo, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, the compound is a compound of Formula (Ia′-2), R⁵ is —F, R⁶ is —OH, and R² is a group selected from any one of the groups listed in Table 2

In some embodiments, the compound is a compound of Formula (Ia′-2), R⁵ is —F, R⁶ is —OH, and R² is —N(R¹²)(R¹³). In some embodiments, the compound is a compound of Formula (Ia′-2), R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments, the compound is a compound of Formula (Ia′-2), R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}; each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆ heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, and —S(O)₂R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen; R²¹ is C₁₋₆alkyl; and R²⁵ is C₁₋₆alkyl. In some embodiments, the compound is a compound of Formula (Ia′-2), R⁵ is —F, R⁶ is —OH, and R² is a group selected from any groups listed in Table 2.

In some embodiments, the compound is a compound of Formula (Ia′-2), R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉ heteroaryl are optionally substituted with one, two, or three R^{20b}. In some embodiments, the compound is a compound of Formula (Ia′-2), R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R²¹ is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂; each R²² is independently C₁₋₆alkyl; each R²³ is C₁₋₆alkyl; and each R²⁵ is selected from C₁₋₆alkyl, C₃₋₆cycloalkyl, and —CH₂—C₃₋₆cycloalkyl. In some embodiments, the compound is a compound of Formula (Ia′-2), R⁵ is —F, R⁶ is —OH, and R² is a group selected from Table 2.

In some embodiments, the compound is a compound of Formula (Ia′-2), R⁵ is —F, R⁶ is —OH, and R² is independently selected from —C(O)OR¹² and —C(O)N(R¹²)(R¹³). In some embodiments, the compound is a compound of Formula (Ia′-2), R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia′-2), R⁵ is —F, R⁶ is —OH, and R³ is independently selected from —C(O)OR¹² and —C(O)N(R¹²)(R¹³). In some embodiments, the compound is a compound of Formula (Ia′-2), R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia′-2), R⁵ is —F, R⁶ is —OH, and R² is a group selected from Table 2.

In some embodiments, the compound is a compound of Formula (Ia′-2), R⁵ is —F, R⁶ is —OH, and R² is selected from

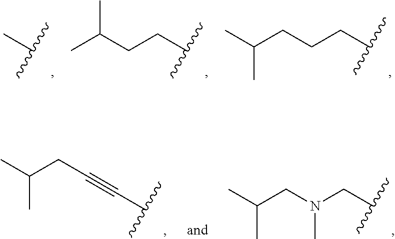

each of which is optionally substituted with one, two, or three R^{20b}. In some embodiments, the compound is a compound of Formula (Ia′-2), R⁵ is —F, R⁶ is —OH, and R² is

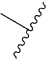

optionally substituted with one, two, or three R^{20b}. In some embodiments, the compound is a compound of Formula (Ia′-2), R⁵ is —F, R⁶ is —OH, and R² is

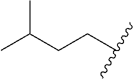 [SMILES: *CCC(C)C]

optionally substituted with one, two, or three R^{20b}. In some embodiments, the compound is a compound of Formula (Ia′-2), R⁵ is —F, R⁶ is —OH, and R² is

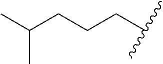 [SMILES: *CCCC(C)C]

optionally substituted with one, two, or three R^{20b}. In some embodiments, the compound is a compound of Formula (Ia′-2), R⁵ is —F, R⁶ is —OH, and R² is

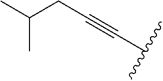 [SMILES: *C#CCC(C)C]

optionally substituted with one, two, or three R^{20b}. In some embodiments, the compound is a compound of Formula (Ia′-2), R⁵ is —F, R⁶ is —OH, and R² is

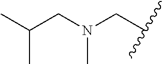 [SMILES: *CN(C)CC(C)C]

optionally substituted with one, two, or three R^{20b}.

In some embodiments, the compound is a compound of Formula (Ia′-2), R⁵ is —F, R⁶ is —OH, and R² is

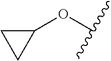 [SMILES: *OC1CC1]

optionally substituted with one, two, or three R^{20k}. In some embodiments, the compound is a compound of Formula (Ia′-2), R⁵ is —F, R⁶ is —OH, and R² is

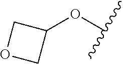 [SMILES: *OC1COC1]

optionally substituted with one, two, or three R^{20k}. In some embodiments, the compound is a compound of Formula (Ia′-2), R⁵ is —F, R⁶ is —OH, and R² is,

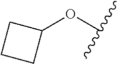 [SMILES: *OC1CCC1]

optionally substituted with one, two, or three R^{20k}. In some embodiments, the compound is a compound of Formula (Ia′-2), R⁵ is —F, R⁶ is —OH, and R² is

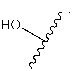 [SMILES: *O]

In some embodiments, the compound is a compound of Formula (Ia′-2), R⁵ is —F, R⁶ is —OH, and R² is

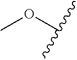 [SMILES: *OC]

optionally substituted with one, two, or three R^{20k}. In some embodiments, the compound is a compound of Formula (Ia′-2), R⁵ is —F, R⁶ is —OH, and R² is

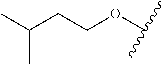 [SMILES: *OCCC(C)C]

optionally substituted with one, two, or three R^{20k}. In some embodiments, the compound is a compound of Formula (Ia′-2), R⁵ is —F, R⁶ is —OH, and R² is

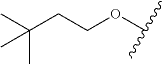 [SMILES: *OCCC(C)(C)C]

optionally substituted with one, two, or three R^{20k}. In some embodiments, the compound is a compound of Formula (Ia′-2), R⁵ is —F, R⁶ is —OH, and R² is

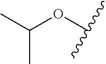 [SMILES: *OC(C)C]

optionally substituted with one, two, or three R^{20k}. In some embodiments, the compound is a compound of Formula (Ia′-2), R⁵ is —F, R⁶ is —OH, and R² is

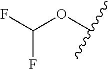 [SMILES: *OC(F)F]

optionally substituted with one R^{20k}. In some embodiments, the compound is a compound of Formula (Ia′-2), R⁵ is —F, R⁶ is —OH, and R² is

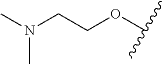 [SMILES: *OCCN(C)C]

optionally substituted with one, two, or three R^{20k}. In some embodiments, the compound is a compound of Formula (Ia′-2), R⁵ is —F, R⁶ is —OH, and R² is

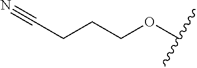 [SMILES: *OCCCC#N]

optionally substituted with one, two, or three R^{20k}. In some embodiments, the compound is a compound of Formula (Ia′-2), R⁵ is —F, R⁶ is —OH, and R² is

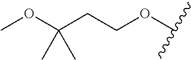 [SMILES: *OCCC(C)(C)OC]

optionally substituted with one, two, or three R^{20k}. In some embodiments, the compound is a compound of Formula (Ia′-2), R⁵ is —F, R⁶ is —OH, and R² is

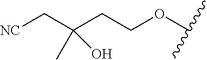 [SMILES: *OCCC(C)(O)CC#N]

optionally substituted with one, two, or three R^{20k}. In some embodiments, the compound is a compound of Formula (Ia′-2), R⁵ is —F, R⁶ is —OH, and R² is

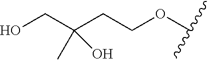 [SMILES: *OCCC(C)(O)CO]

optionally substituted with one, two, or three R^{20k}. In some embodiments, the compound is a compound of Formula (Ia′-2), R⁵ is —F, R⁶ is —OH, and R² is

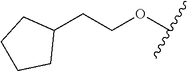 [SMILES: *OCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

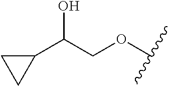 [SMILES: *OCC(O)C1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

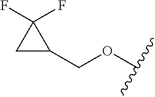 [SMILES: *OCC1CC1(F)F]

optionally substituted with one, two, or three R^{20k}. In some embodiments, the compound is a compound of Formula (Ia′-2), R⁵ is —F, R⁶ is —OH, and R² is

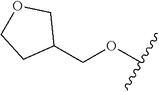 [SMILES: *OCC1CCOC1]

optionally substituted with one, two, or three R^{20k}. In some embodiments, the compound is a compound of Formula (Ia′-2), R⁵ is —F, R⁶ is —OH, and R² is

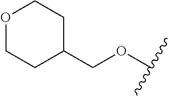 [SMILES: *OCC1CCOCC1]

optionally substituted with one, two, or three R^{20k}. In some embodiments, the compound is a compound of Formula (Ia′-2), R⁵ is —F, R⁶ is —OH, and R² is

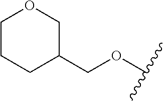 [SMILES: *OCC1CCCOC1]

optionally substituted with one, two, or three R^{20k}. In some embodiments, the compound is a compound of Formula (Ia′-2), R⁵ is —F, R⁶ is —OH, and R² is

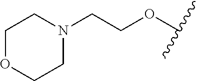 [SMILES: *OCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

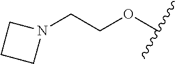 [SMILES: *OCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

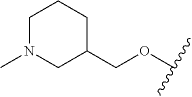 [SMILES: *OCC1CCCN(C)C1]

optionally substituted with one, two, or three R^{20k} In some embodiments, the compound is a compound of Formula (Ia′-2), R⁵ is —F, R⁶ is —OH, and R² is

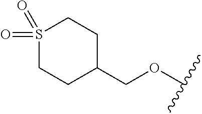 [SMILES: *OCC1CCS(=O)(=O)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

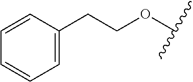 [SMILES: *OCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

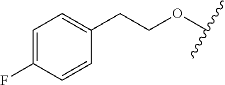 [SMILES: *OCCc1ccc(F)cc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

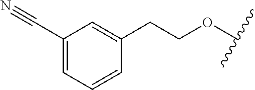 [SMILES: *OCCc1cccc(C#N)c1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia′-2), R⁵ is —F, R⁶ is —OH, and R² is

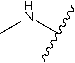 [SMILES: *NC]

optionally substituted with one, two, or three R^{20k}. In some embodiments, the compound is a compound of Formula (Ia′-2), R⁵ is —F, R⁶ is —OH, and R² is

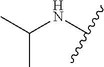 [SMILES: *NC(C)C]

optionally substituted with one, two, or three R^{20k}. In some embodiments, the compound is a compound of Formula (Ia′-2), R⁵ is —F, R⁶ is —OH, and R² is

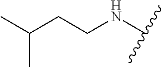 [SMILES: *NCCC(C)C]

optionally substituted with one, two, or three R^{20k}. In some embodiments, the compound is a compound of Formula (Ia′-2), R⁵ is —F, R⁶ is —OH, and R² is

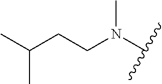 [SMILES: *N(C)CCC(C)C]

optionally substituted with one, two, or three R^{20k}. In some embodiments, the compound is a compound of Formula (Ia′-2), R⁵ is —F, R⁶ is —OH, and R² is

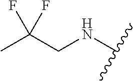 [SMILES: *NCC(C)(F)F]

optionally substituted with one, two, or three R^{20k}. In some embodiments, the compound is a compound of Formula (Ia′-2), R⁵ is —F, R⁶ is —OH, and R² is

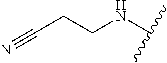 [SMILES: *NCCC#N]

optionally substituted with one, two, or three R^{20k}. In some embodiments, the compound is a compound of Formula (Ia′-2), R⁵ is —F, R⁶ is —OH, and R² is

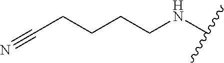 [SMILES: *NCCCCC#N]

optionally substituted with one, two or three R^{20k}. In some embodiments, the compound is a compound of Formula (Ia′-2), R⁵ is —F, R⁶ is —OH, and R² is

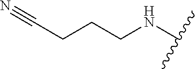 [SMILES: *NCCCC#N]

optionally substituted with one, two, or three R^{20k}. In some embodiments, the compound is a compound of Formula (Ia′-2), R⁵ is —F, R⁶ is —OH, and R² is

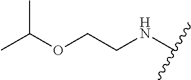 [SMILES: *NCCOC(C)C]

optionally substituted with one, two, or three R^{20k}. In some embodiments, the compound is a compound of Formula (Ia′-2), R⁵ is —F, R⁶ is —OH, and R² is

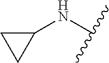 [SMILES: *NC1CC1]

optionally substituted with one, two, or three R^{20k}. In some embodiments, the compound is a compound of Formula (Ia′-2), R⁵ is —F, R⁶ is —OH, and R² is

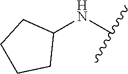 [SMILES: *NC1CCCC1]

optionally substituted with one, two, or three R^{20k}. In some embodiments, the compound is a compound of Formula (Ia′-2), R⁵ is —F, R⁶ is —OH, and R² is

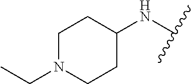 [SMILES: *NC1CCN(CC)CC1]

optionally substituted with one, two, or three R^{20k}. In some embodiments, the compound is a compound of Formula (Ia′-2), R⁵ is —F, R⁶ is —OH, and R² is

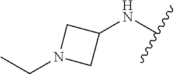 [SMILES: *NC1CN(CC)C1]

optionally substituted with one, two, or three R^{20k}. In some embodiments, the compound is a compound of Formula (Ia′-2), R⁵ is —F, R⁶ is —OH, and R² is

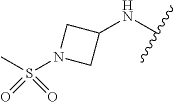 [SMILES: *NC1CN(S(C)(=O)=O)C1]

optionally substituted with one, two, or three R^{20k}. In some embodiments, the compound is a compound of Formula (Ia′-2), R⁵ is —F, R⁶ is —OH, and R² is

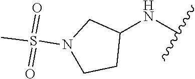 [SMILES: *NC1CCN(S(C)(=O)=O)C1]

optionally substituted with one, two, or three R^{20k}. In some embodiments, the compound is a compound of Formula (Ia′-2), R⁵ is —F, R⁶ is —OH, and R² is

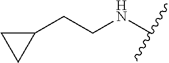 [SMILES: *NCCC1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

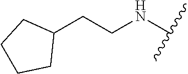 [SMILES: *NCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

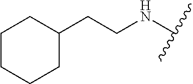 [SMILES: *NCCC1CCCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

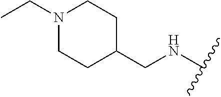 [SMILES: *NCC1CCN(CC)CC1]

optionally substituted with one, two, or three R^{20k}. In some embodiments, the compound is a compound of Formula (Ia′-2), R⁵ is —F, R⁶ is —OH, and R² is

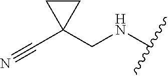 [SMILES: *NCC1(C#N)CC1]

optionally substituted with one, two, or three R^{20k}. In some embodiments, the compound is a compound of Formula (Ia′-2), R⁵ is —F, R⁶ is —OH, and R² is

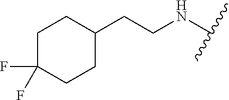 [SMILES: *NCCC1CCC(F)(F)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

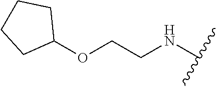 [SMILES: *NCCOC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

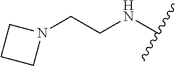 [SMILES: *NCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

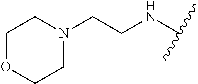 [SMILES: *NCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

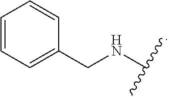 [SMILES: *NCc1ccccc1]

In some embodiments, the compound is a compound of Formula (Ia′-2), R⁵ is —F, R⁶ is —OH, and R² is

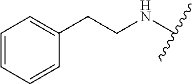 [SMILES: *NCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

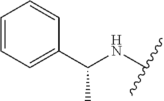 [SMILES: *N[C@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

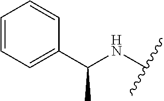 [SMILES: *N[C@@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

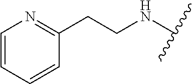 [SMILES: *NCCc1ccccn1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia′-2), R⁵ is —F, R⁶ is —OH, and R² is

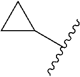 [SMILES: *C1CC1]

optionally substituted with one, two, or three R^{20b}. In some embodiments, the compound is a compound of Formula (Ia′-2), R⁵ is —F, R⁶ is —OH, and R² is

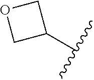 [SMILES: *C1COC1]

optionally substituted with one, two, or three R^{20b}. In some embodiments, the compound is a compound of Formula (Ia′-2), R⁵ is —F, R⁶ is —OH, and R² is

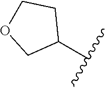 [SMILES: *C1CCOC1]

optionally substituted with one, two, or three R^{20b}. In some embodiments, the compound is a compound of Formula (Ia′-2), R⁵ is —F, R⁶ is —OH, and R² is

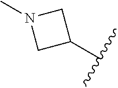 [SMILES: *C1CN(C)C1]

optionally substituted with one, two, or three R^{20b} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

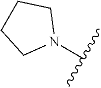 [SMILES: *N1CCCC1]

optionally substituted with one, two, or three R^{20b}. In some embodiments, the compound is a compound of Formula (Ia′-2), R⁵ is —F, R⁶ is —OH, and R² is

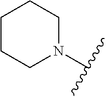 [SMILES: *N1CCCCC1]

optionally substituted with one, two, or three R^{20b}. In some embodiments, the compound is a compound of Formula (Ia′-2), R⁵ is —F, R⁶ is —OH, and R² is

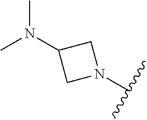 [SMILES: *N1CC(N(C)C)C1]

optionally substituted with one, two, or three R^{20b}. In some embodiments, the compound is a compound of Formula (Ia′-2), R⁵ is —F, R⁶ is —OH, and R² is

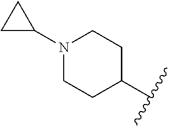 [SMILES: *C1CCN(C2CC2)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

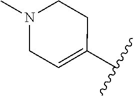 [SMILES: *C1=CCN(C)CC1]

optionally substituted with one, two, or three R²¹ and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R² is

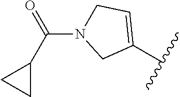 [SMILES: *C1=CCN(C(=O)C2CC2)C1]

optionally substituted with one, two, or three R^{20b}. In some embodiments, the compound is a compound of Formula (Ia′-2), R⁵ is —F, R⁶ is —OH, and R² is

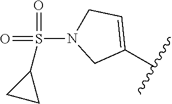 [SMILES: *C1=CCN(S(=O)(=O)C2CC2)C1]

optionally substituted with one, two, or three R^{20b}. In some embodiments, the compound is a compound of Formula (Ia′-2), R⁵ is —F, R⁶ is —OH, and R² is

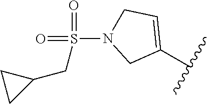 [SMILES: *C1=CCN(S(=O)(=O)CC2CC2)C1]

optionally substituted with one, two, or three R^{20b} In some embodiments, the compound is a compound of Formula (Ia′-2), R⁵ is —F, R⁶ is —OH, and R² is

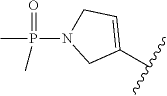 [SMILES: *C1=CCN(P(C)(C)=O)C1]

optionally substituted with one, two, or three R^{20b}. In some embodiments, the compound is a compound of Formula (Ia′-2), R⁵ is —F, R⁶ is —OH, and R² is

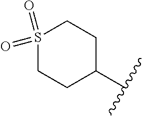 [SMILES: *C1CCS(=O)(=O)CC1]

optionally substituted with one, two, or three R^{20b}. In some embodiments, the compound is a compound of Formula (Ia′-2), R⁵ is —F, R⁶ is —OH, and R² is

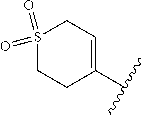 [SMILES: *C1=CCS(=O)(=O)CC1]

optionally substituted with one, two, or three R^{20b}. In some embodiments, the compound is a compound of Formula (Ia′-2), R⁵ is —F, R⁶ is —OH, and R² is

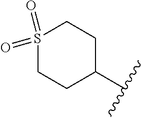

optionally substituted with one, two, or three R^{20b}. In some embodiments, the compound is a compound of Formula (Ia′-2), R⁵ is —F, R⁶ is —OH, and R² is

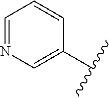 [SMILES: *c1cccnc1]

optionally substituted with one, two, or three R^{20b}. In some embodiments, the compound is a compound of Formula (Ia′-2), R⁵ is —F, R⁶ is —OH, and R² is

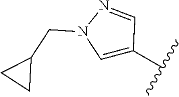 [SMILES: *c1cnn(CC2CC2)c1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia′-2), R⁵ is —F, R⁶ is —OH, and R² is independently hydrogen. In some embodiments, the compound is a compound of Formula (Ia′-2), R⁵ is —F, R⁶ is —OH, and R² is independently halogen. In some embodiments, the compound is a compound of Formula (Ia′-2), R⁵ is —F, R⁶ is —OH, and R² is independently —CN. In some embodiments, the compound is a compound of Formula (Ia′-2), R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments, the compound is a compound of Formula (Ia′-2), R⁵ is —F, R⁶ is —OH, and R² is independently —OR¹². In some embodiments, the compound is a compound of Formula (Ia′-2), R⁵ is —F, R⁶ is —OH, and R² is independently —SR¹². In some embodiments, the compound is a compound of Formula (Ia′-2), R⁵ is —F, R⁶ is —OH, and R² is independently —N(R¹²)(R¹³). In some embodiments, the compound is a compound of Formula (Ia′-2), R⁵ is —F, R⁶ is —OH, and R² is independently —C(O)OR¹². In some embodiments, the compound is a compound of Formula (Ia′-2), R⁵ is —F, R⁶ is —OH, and R² is independently —OC(O)N(R¹²)(R¹³). In some embodiments, the compound is a compound of Formula (Ia′-2), R⁵ is —F, R⁶ is —OH, and R² is independently —N(R¹⁴)C(O)N(R¹²)(R¹³). In some embodiments, the compound is a compound of Formula (Ia′-2), R⁵ is —F, R⁶ is —OH, and R² is independently —N(R¹⁴)C(O)OR¹². In some embodiments, the compound is a compound of Formula (Ia′-2), R⁵ is —F, R⁶ is —OH, and R² is independently —N(R¹⁴)S(O)₂R¹⁵. In some embodiments, the compound is a compound of Formula (Ia′-2), R⁵ is —F, R⁶ is —OH, and R² is independently —C(O)R¹⁵. In some embodiments, the compound is a compound of Formula (Ia′-2), R⁵ is —F, R⁶ is —OH, and R² is independently —S(O)R¹⁵. In some embodiments, the compound is a compound of Formula (Ia′-2), R⁵ is —F, R⁶ is —OH, and R² is independently —OC(O)R¹⁵. In some embodiments, the compound is a compound of Formula (Ia′-2), R⁵ is —F, R⁶ is —OH, and R² is independently —C(O)N(R¹²)(R¹³). In some embodiments, the compound is a compound of Formula (Ia′-2), R⁵ is —F, R⁶ is —OH, and R² is independently —C(O)C(O)N(R¹²)(R¹³). In some embodiments, the compound is a compound of Formula (Ia′-2), R⁵ is —F, R⁶ is —OH, and R² is independently —N(R¹⁴)C(O)R¹⁵. In some embodiments, the compound is a compound of Formula (Ia′-2), R⁵ is —F, R⁶ is —OH, and R² is independently —S(O)₂R¹⁵. In some embodiments, the compound is a compound of Formula (Ia′-2), R⁵ is —F, R⁶ is —OH, and R² is independently —S(O)₂N(R¹²)(R¹³). In some embodiments, the compound is a compound of Formula (Ia′-2), R⁵ is —F, R⁶ is —OH, and R² is independently —S(═O)(═NH)N(R¹²)(R¹³). In some embodiments, the compound is a compound of Formula (Ia′-2), R⁵ is —F, R⁶ is —OH, and R² is independently —CH₂C(O)N(R¹²)(R¹³). In some embodiments, the compound is a compound of Formula (Ia′-2), R⁵ is —F, R⁶ is —OH, and R² is independently —CH₂N(R¹⁴)C(O)R¹⁵. In some embodiments, the compound is a compound of Formula (Ia′-2), R⁵ is —F, R⁶ is —OH, and R² is independently —CH₂S(O)₂R¹⁵. In some embodiments, the compound is a compound of Formula (Ia′-2), R⁵ is —F, R⁶ is —OH, and R² is independently —CH₂S(O)₂N(R¹²)(R¹³). In some embodiments, the compound is a compound of Formula (Ia′-2), R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20b}. In some embodiments, the compound is a compound of Formula (Ia′-2), R⁵ is —F, R⁶ is —OH, and R² is independently C₁₋₆alkyl substituted with one, two, or three R^{20b}. In some embodiments, the compound is a compound of Formula (Ia′-2), R⁵ is —F, R⁶ is —OH, and R² is independently C₂₋₆alkenyl substituted with one, two, or three R^{20b}. In some embodiments, the compound is a compound of Formula (Ia′-2), R⁵ is —F, R⁶ is —OH, and R² is independently C₂₋₆alkynyl substituted with one, two, or three R^{20b}. In some embodiments, the compound is a compound of Formula (Ia′-2), R⁵ is —F, R⁶ is —OH, and R² is independently C₃₋₆cycloalkyl substituted with one, two, or three R^{20b}. In some embodiments, the compound is a compound of Formula (Ia′-2), R⁵ is —F, R⁶ is —OH, and R² is independently C₂₋₉heterocycloalkyl substituted with one, two, or three R^{20b}. In some embodiments, the compound is a compound of Formula (Ia′-2), R⁵ is —F, R⁶ is —OH, and R² is independently C₆₋₁₀aryl substituted with one, two, or three R^{20b}. In some embodiments, the compound is a compound of Formula (Ia′-2), R⁵ is —F, R⁶ is —OH, and R² is independently C₁₋₉heteroaryl substituted with one, two, or three R^{20b}. In some embodiments, the compound is a compound of Formula (Ia′-2), R⁵ is —F, R⁶ is —OH, and R² is independently C₁₋₆alkyl. In some embodiments, the compound is a compound of Formula (Ia′-2), R⁵ is —F, R⁶ is —OH, and R² is independently C₂₋₆alkenyl. In some embodiments, the compound is a compound of Formula (Ia′-2), R⁵ is —F, R⁶ is —OH, and R² is independently C₂₋₆alkynyl. In some embodiments, the compound is a compound of Formula (Ia′-2), R⁵ is —F, R⁶ is —OH, and R² is independently C₃₋₆cycloalkyl. In some embodiments, the compound is a compound of Formula (Ia′-2), R⁵ is —F, R⁶ is —OH, and R² is independently C₂₋₉heterocycloalkyl. In some embodiments, the compound is a compound of Formula (Ia′-2), R⁵ is —F, R⁶ is —OH, and R² is independently C₆₋₁₀aryl. In some embodiments, the compound is a compound of Formula (Ia′-2), R⁵ is —F, R⁶ is —OH, and R² is independently C₁₋₉heteroaryl. In some embodiments, the compound is a compound of Formula (Ia′-2), R⁵ is —F, R⁶ is —OH, and R² is independently C₁alkyl. In some embodiments, the compound is a compound of Formula (Ia′-2), R⁵ is —F, R⁶ is —OH, and R² is independently C₂alkyl. In some embodiments, the compound is a compound of Formula (Ia′-2), R⁵ is —F, R⁶ is —OH, and R² is independently C₃alkyl. In some embodiments, the compound is a compound of Formula (Ia′-2), R⁵ is —F, R⁶ is —OH, and R² is independently C₄alkyl. In some embodiments, the compound is a compound of Formula (Ia′-2), R⁵ is —F, R⁶ is —OH, and R² is independently C₅alkyl. In some embodiments, the compound is a compound of Formula (Ia′-2), R⁵ is —F, R⁶ is —OH, and R² is independently C₆alkyl.

In some embodiments, the compound is a compound of Formula (Ia′-2), R⁵ is —F, R⁶ is —OH, and R² is independently —OH. In some embodiments, the compound is a compound of Formula (Ia′-2), R⁵ is —F, R⁶ is —OH, and R² is independently —SH. In some embodiments, the compound is a compound of Formula (Ia′-2), R⁵ is —F, R⁶ is —OH, and R² is independently —NH₂. In some embodiments, the compound is a compound of Formula (Ia′-2), R⁵ is —F, R⁶ is —OH, and R² is independently —C(O)OH. In some embodiments, the compound is a compound of Formula (Ia′-2), R⁵ is —F, R⁶ is —OH, and R² is independently —OC(O)NH₂. In some embodiments, the compound is a compound of Formula (Ia′-2), R⁵ is —F, R⁶ is —OH, and R² is independently —NHC(O)NH₂. In some embodiments, the compound is a compound of Formula (Ia′-2), R⁵ is —F, R⁶ is —OH, and R² is independently —NHC(O)OH. In some embodiments, the compound is a compound of Formula (Ia′-2), R⁵ is —F, R⁶ is —OH, and R² is independently —NHS(O)₂R¹⁵. In some embodiments, the compound is a compound of Formula (Ia′-2), R⁵ is —F, R⁶ is —OH, and R² is independently —C(O)R¹⁵. In some embodiments, the compound is a compound of Formula (Ia′-2), R⁵ is —F, R⁶ is —OH, and R² is independently —S(O)R¹⁵. In some embodiments, the compound is a compound of Formula (Ia′-2), R⁵ is —F, R⁶ is —OH, and R² is independently —OC(O)R¹⁵. In some embodiments, the compound is a compound of Formula (Ia′-2), R⁵ is —F, R⁶ is —OH, and R² is independently —OC(O)CH₃. In some embodiments, the compound is a compound of Formula (Ia′-2), R⁵ is —F, R⁶ is —OH, and R² is independently —C(O)NH₂. In some embodiments, the compound is a compound of Formula (Ia′-2), R⁵ is —F, R⁶ is —OH, and R² is independently —C(O)C(O)NH₂. In some embodiments, the compound is a compound of Formula (Ia′-2), R⁵ is —F, R⁶ is —OH, and R² is independently —NHC(O)R¹⁵. In some embodiments, the compound is a compound of Formula (Ia′-2), R⁵ is —F, R⁶ is —OH, and R² is independently —S(O)₂R¹⁵. In some embodiments, the compound is a compound of Formula (Ia′-2), R⁵ is —F, R⁶ is —OH, and R² is independently —S(O)₂N(R¹²)(R¹³). In some embodiments, the compound is a compound of Formula (Ia′-2), R⁵ is —F, R⁶ is —OH, and R² is independently —S(═O)(═NH)NH₂. In some embodiments, the compound is a compound of Formula (Ia′-2), R⁵ is —F, R⁶ is —OH, and R² is independently —CH₂C(O)NH₂. In some embodiments, the compound is a compound of Formula (Ia′-2), R⁵ is —F, R⁶ is —OH, and R² is independently —CH₂NHC(O)R¹⁵. In some embodiments, the compound is a compound of Formula (Ia′-2), R⁵ is —F, R⁶ is —OH, and R² is independently —CH₂S(O)₂R¹⁵. In some embodiments, the compound is a compound of Formula (Ia′-2), R⁵ is —F, R⁶ is —OH, and R² is independently —CH₂S(O)₂NH₂.

In some embodiments, the compound is a compound of Formula (Ia′-2), R⁵ is —F, R⁶ is —OH, and R² is independently —NH(R¹²). In some embodiments, the compound is a compound of Formula (Ia′-2), R⁵ is —F, R⁶ is —OH, and R² is independently —OC(O)N(R¹²)(R¹³). In some embodiments, the compound is a compound of Formula (Ia′-2), R⁵ is —F, R⁶ is —OH, and R² is independently —NHC(O)N(R¹²). In some embodiments, the compound is a compound of Formula (Ia′-2), R⁵ is —F, R⁶ is —OH, and R² is independently —NHC(O)OR¹². In some embodiments, the compound is a compound of Formula (Ia′-2), R⁵ is —F, R⁶ is —OH, and R² is independently —C(O)N(R¹²)(R¹³). In some embodiments, the compound is a compound of Formula (Ia′-2), R⁵ is —F, R⁶ is —OH, and R² is independently —C(O)C(O)NH(R¹²). In some embodiments, the compound is a compound of Formula (Ia′-2), R⁵ is —F, R⁶ is —OH, and R² is independently —S(O)₂NH(R¹²). In some embodiments, the compound is a compound of Formula (Ia′-2), R⁵ is —F, R⁶ is —OH, and R² is independently —S(═O)(═NH)NH(R¹²). In some embodiments, the compound is a compound of Formula (Ia′-2), R⁵ is —F, R⁶ is —OH, and R² is independently —CH₂C(O)NH(R¹²). In some embodiments, the compound is a compound of Formula (Ia′-2), R⁵ is —F, R⁶ is —OH, and R² is independently —CH₂S(O)₂NH(R¹²).

In some embodiments, the compound is a compound of Formula (Ia′-2), R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In some embodiments, the compound is a compound of Formula (Ia′-2), R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}.

In some embodiments, the compound is a compound of Formula (Ia′-2), R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

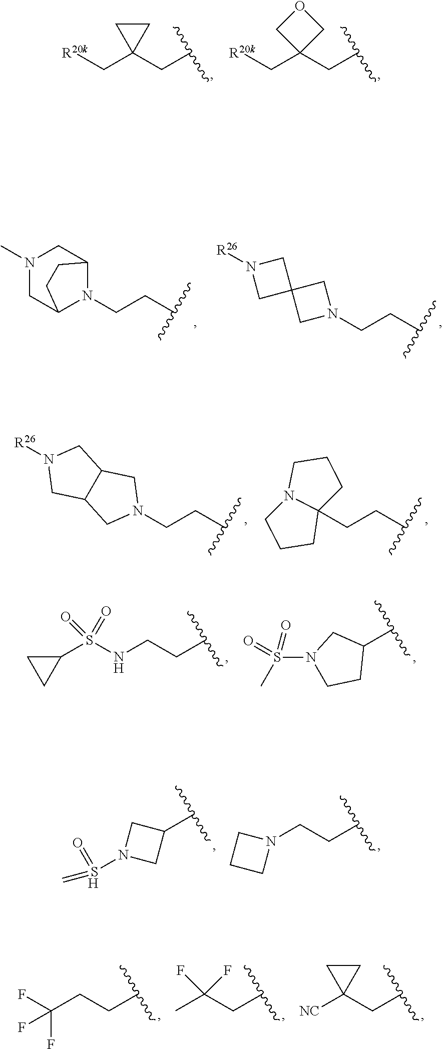

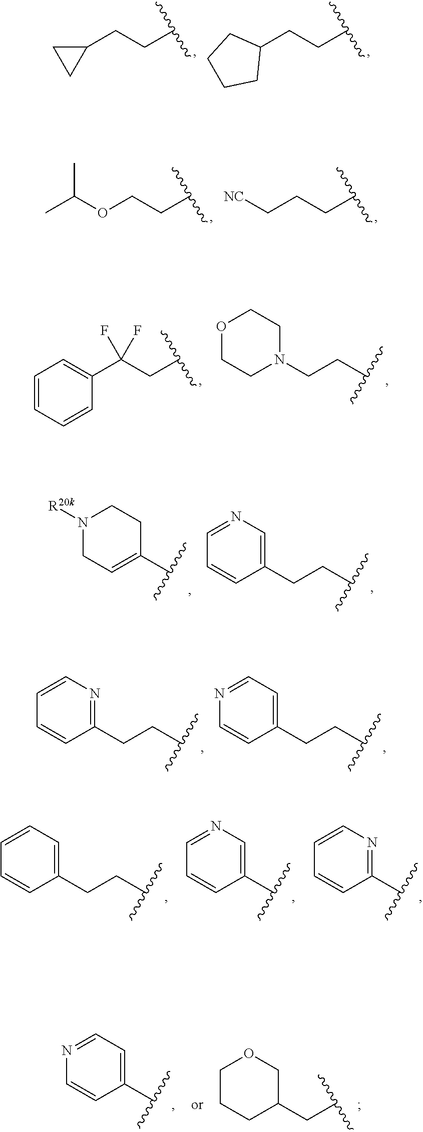

R¹⁵ is independently

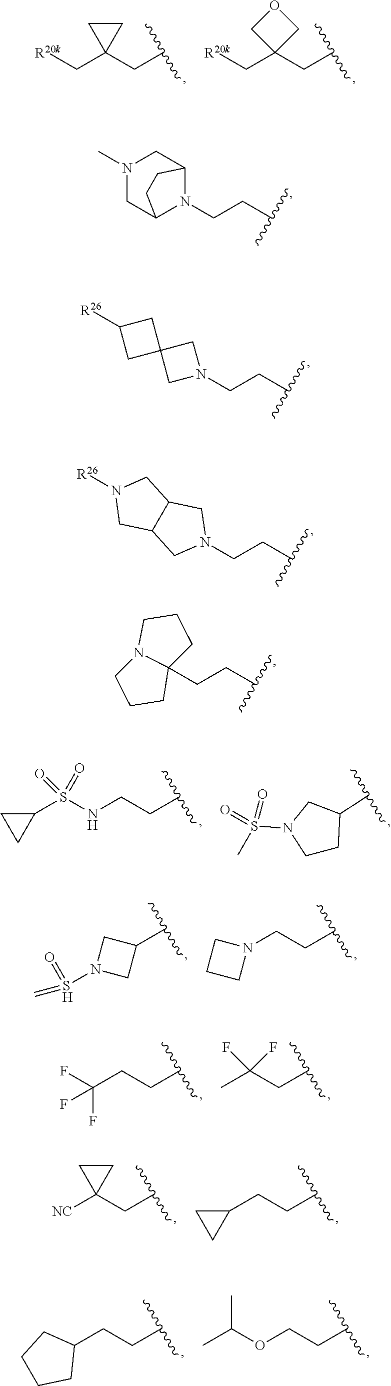

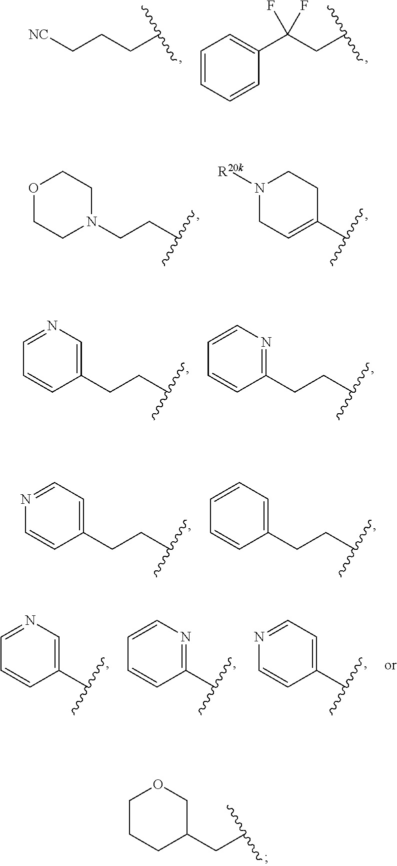

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia′-2), R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

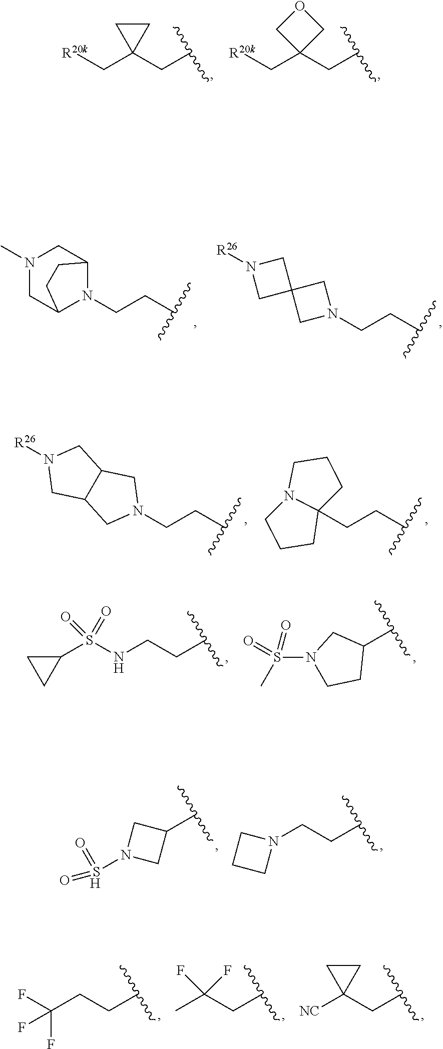

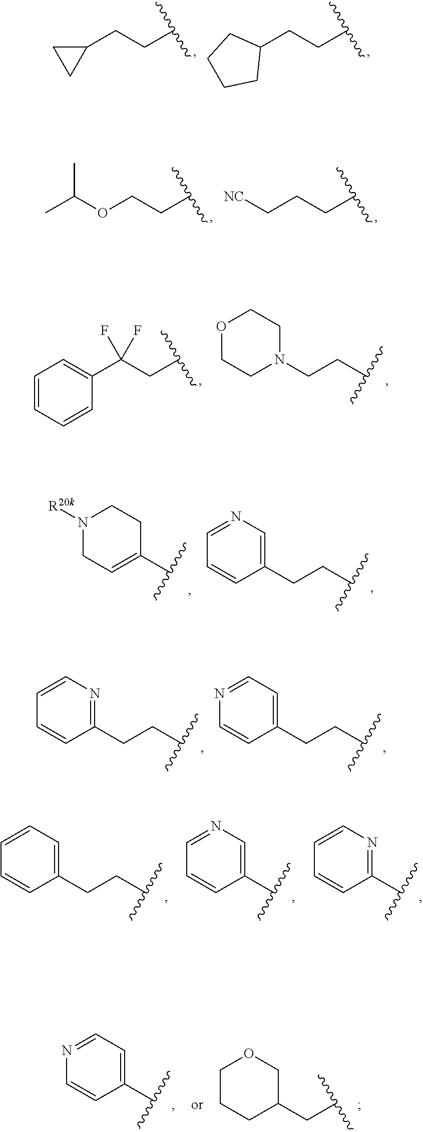

R¹⁵ is independently

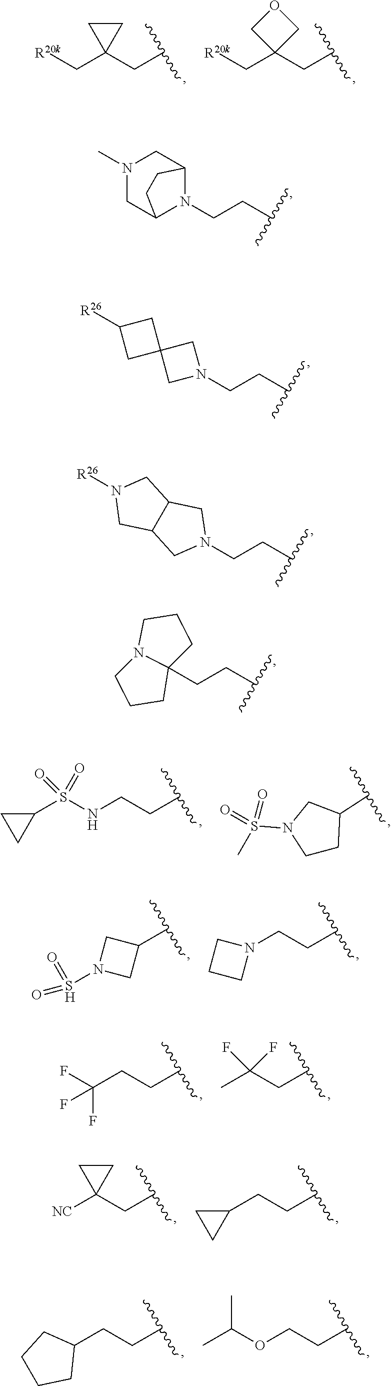

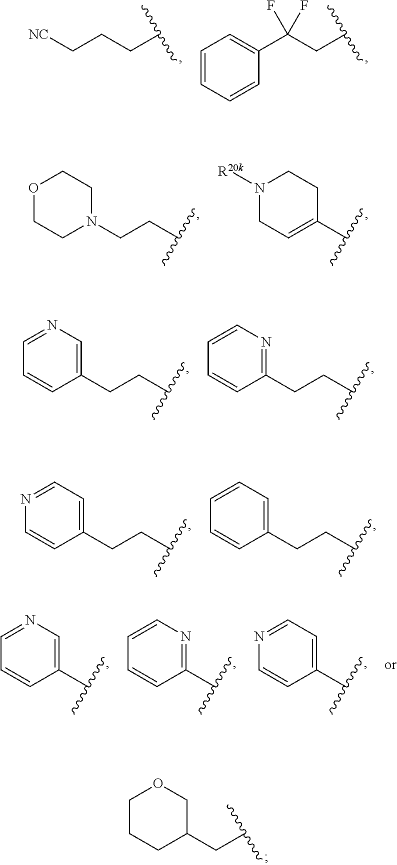

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia′-2), R⁵ is —F, R⁶ is —OH, and R² is selected from

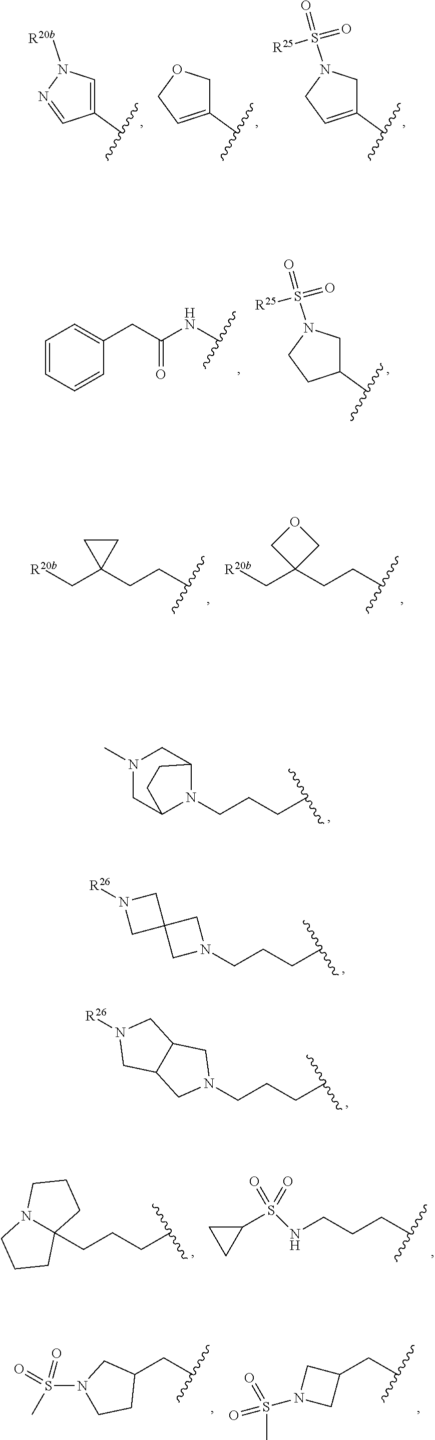

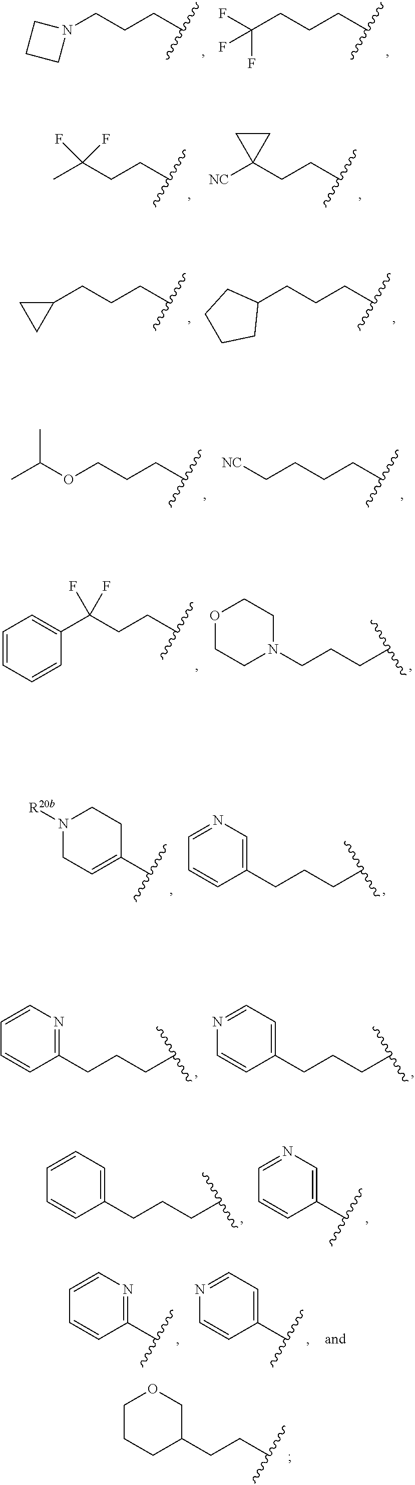

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia′-2), R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia′-2), R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² and R¹⁵ are independently

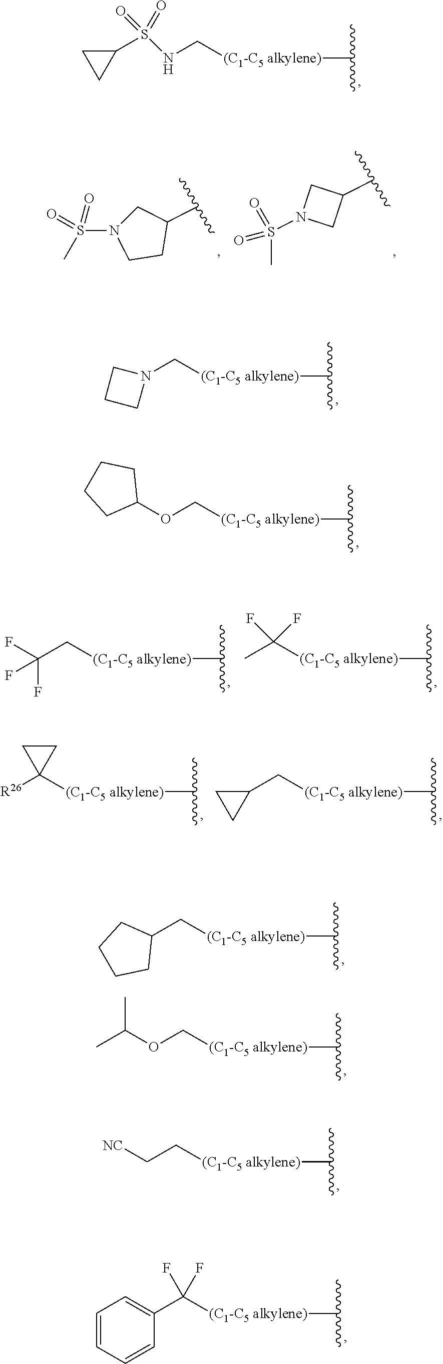

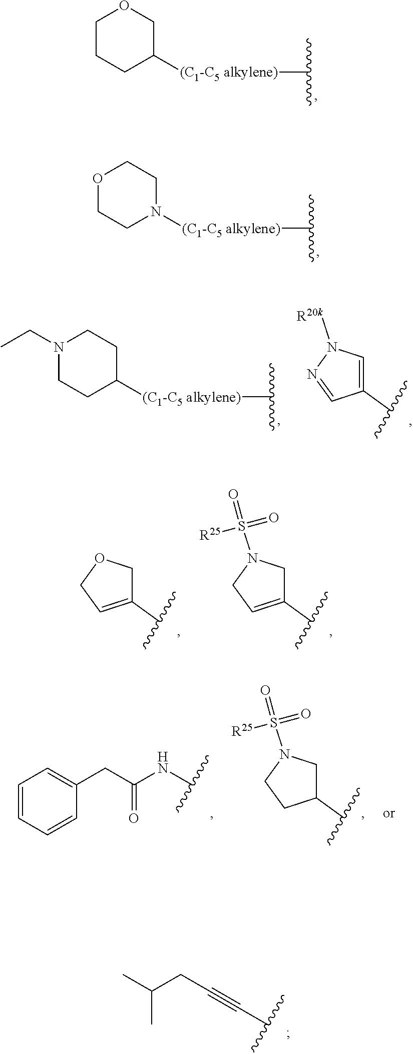

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia′-2), R⁵ is —F, R⁶ is —OH, and R² is independently

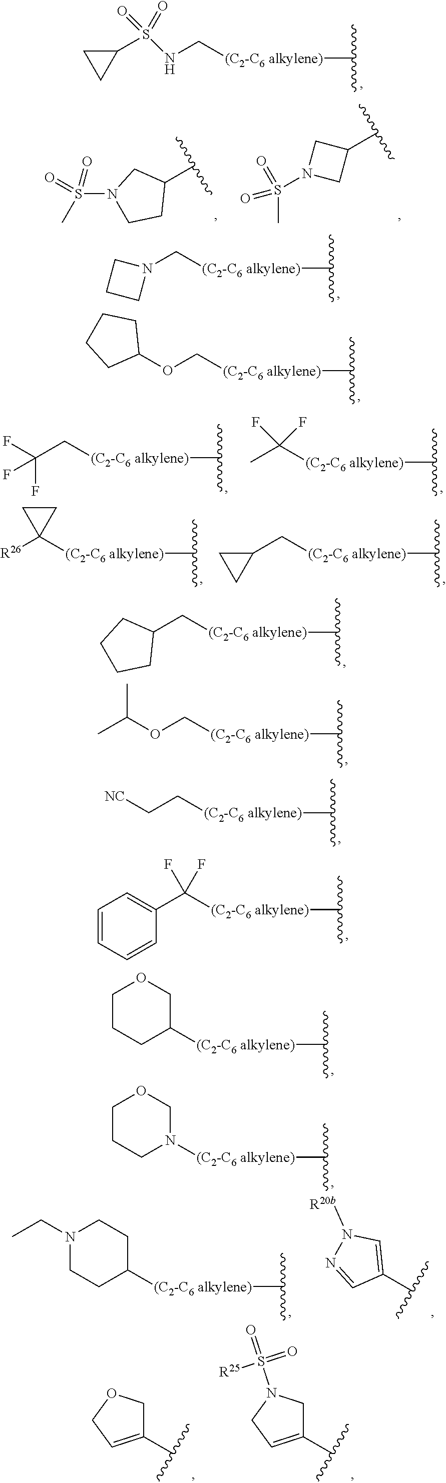

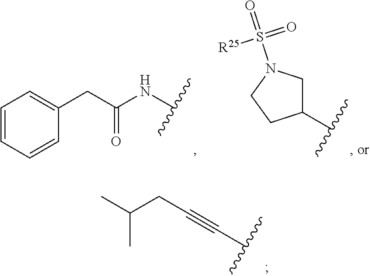

and R²⁶ is independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2):

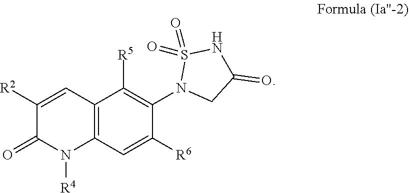

In some embodiments, the compound is a compound of Formula (Ia″-2), R⁴ is methyl, R⁵ is —F, R⁶ is —OH, and R² is C₁₋₆ alkyl, optionally substituted with one, two, or three R^{20b}. In some embodiments, the compound is a compound of Formula (Ia″-2), R⁴ is methyl, R⁵ is —F, R⁶ is —OH, and R² is C₁₋₅alkyl, optionally substituted with one, two, or three R^{20b}. In some embodiments, the compound is a compound of Formula (Ia″-2), R⁴ is methyl, R⁵ is —F, R⁶ is —OH, and R² is C₁₋₄alkyl, optionally substituted with one, two, or three R²¹. In some embodiments, the compound is a compound of Formula (Ia″-2), R⁴ is methyl, R⁵ is —F, R⁶ is —OH, and R² is C₁₋₃alkyl, optionally substituted with one, two, or three R^{20b}. In some embodiments, the compound is a compound of Formula (Ia″-2), R⁴ is methyl, R⁵ is —F, R⁶ is —OH, and R² is C₁₋₂alkyl, optionally substituted with one, two, or three R²¹. In some embodiments, the compound is a compound of Formula (Ia″-2), R⁴ is methyl, R⁵ is —F, R⁶ is —OH, and R² is C₁alkyl, optionally substituted with one, two, or three R^{20b}. In some embodiments, the compound is a compound of Formula (Ia″-2), R⁴ is methyl, R⁵ is —F, R⁶ is —OH, and R² is C₂alkyl, optionally substituted with one, two, or three R^{20b}. In some embodiments, the compound is a compound of Formula (Ia″-2), R⁴ is methyl, R⁵ is —F, R⁶ is —OH, and R² is C₃alkyl, optionally substituted with one, two, or three R^{20b}. In some embodiments, the compound is a compound of Formula (Ia″-2), R⁴ is methyl, R⁵ is —F, R⁶ is —OH, and R² is C₄alkyl, optionally substituted with one, two, or three R^{20b}. In some embodiments, the compound is a compound of Formula (Ia″-2), R⁴ is methyl, R⁵ is —F, R⁶ is —OH, and R² is C₅alkyl, optionally substituted with one, two, or three R^{20b}. In some embodiments, the compound is a compound of Formula (Ia″-2), R⁴ is methyl, R⁵ is —F, R⁶ is —OH, and R² is C₆alkyl, optionally substituted with one, two, or three R²¹. In some embodiments, the compound is a compound of Formula (Ia″-2), R⁴ is methyl, R⁵ is —F, R⁶ is —OH, and each R^{20b} is independently selected from —N(R²²)(R²³), R²² is C₁₋₆alkyl, and R²³ is C₁₋₆alkyl. In some embodiments, the compound is a compound of Formula (Ia″-2), R⁴ is methyl, R⁵ is —F, R⁶ is —OH, and R² is C₂₋₆alkynyl, optionally substituted with one, two, or three R^{20b}. In some embodiments, the compound is a compound of Formula (Ia″-2), R⁴ is methyl, R⁵ is —F, R⁶ is —OH, and R² is selected from any group listed in Table 2

In some embodiments, the compound is a compound of Formula (Ia″-2), R⁴ is methyl, R⁵ is —F, R⁶ is —OH, and R² is —OR¹². In some embodiments, the compound is a compound of Formula (Ia″-2), R⁴ is methyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}. In some embodiments, the compound is a compound of Formula (Ia″-2), R⁴ is methyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆ alkyl, and R²³ is C₁₋₆alkyl. In some embodiments, the compound is a compound of Formula (Ia″-2), R⁴ is methyl, R⁵ is —F, R⁶ is —OH, and R² is —OR¹² and R¹² is independently selected from C₁₋₆alkyl, C₃₄cycloalkyl, C₃₋₅heterocycloalkyl, and —CH₂—C₄₋₅heterocycloalkyl, wherein C₁₋₆alkyl, C₃₄cycloalkyl, C₃₋₅heterocycloalkyl, and —CH₂—C₄₋₅heterocycloalkyl, are optionally substituted with one, two, or three R^{20k}; and each R^{20k} is independently selected from oxo, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, the compound is a compound of Formula (Ia″-2), R⁴ is methyl, R⁵ is —F, R⁶ is —OH, and R² is a group selected from any one of the groups listed in Table 2

In some embodiments, the compound is a compound of Formula (Ia″-2), R⁴ is methyl, R⁵ is —F, R⁶ is —OH, and R² is —N(R¹²)(R¹³). In some embodiments, the compound is a compound of Formula (Ia″-2), R⁴ is methyl, R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments, the compound is a compound of Formula (Ia″-2), R⁴ is methyl, R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}; each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, and —S(O)₂R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen; R²¹ is C₁₋₆alkyl; and R²⁵ is C₁₋₆alkyl. In some embodiments, the compound is a compound of Formula (Ia″-2), R⁴ is methyl, R⁵ is —F, R⁶ is —OH, and R² is a group selected from any groups listed in Table 2.

In some embodiments, the compound is a compound of Formula (Ia″-2), R⁴ is methyl, R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉ heteroaryl are optionally substituted with one, two, or three R^{20b}. In some embodiments, the compound is a compound of Formula (Ia″-2), R⁴ is methyl, R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂; each R²² is independently C₁₋₆alkyl; each R²³ is C₁₋₆alkyl; and each R²⁵ is selected from C₁₋₆alkyl, C₃₋₆cycloalkyl, and —CH₂—C₃₋₆cycloalkyl. In some embodiments, the compound is a compound of Formula (Ia″-2), R⁴ is methyl, R⁵ is —F, R⁶ is —OH, and R² is a group selected from Table 2.\

In some embodiments, the compound is a compound of Formula (Ia″-2), R⁴ is methyl, R⁵ is —F, R⁶ is —OH, and R² is independently selected from —C(O)OR¹² and —C(O)N(R¹²)(R¹³). In some embodiments, the compound is a compound of Formula (Ia″-2), R⁴ is methyl, R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia″-2), R⁴ is methyl, R⁵ is —F, R⁶ is —OH, and R³ is independently selected from —C(O)OR¹² and —C(O)N(R¹²)(R¹³). In some embodiments, the compound is a compound of Formula (Ia″-2), R⁴ is methyl, R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia″-2), R⁴ is methyl, R⁵ is —F, R⁶ is —OH, and R² is a group selected from Table 2.

In some embodiments, the compound is a compound of Formula (Ia″-2), R⁴ is methyl, R⁵ is —F, R⁶ is —OH, and R² is selected from

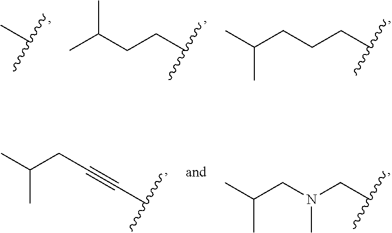

each of which is optionally substituted with one, two, or three R^{20b}. In some embodiments, the compound is a compound of Formula (Ia″-2), R⁴ is methyl, R⁵ is —F, R⁶ is —OH, and R² is

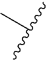

optionally substituted with one, two, or three R^{20b}. In some embodiments, the compound is a compound of Formula (Ia″-2), R⁴ is methyl, R⁵ is —F, R⁶ is —OH, and R² is

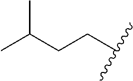 [SMILES: *CCC(C)C]

optionally substituted with one, two, or three R^{20b}. In some embodiments, the compound is a compound of Formula (Ia″-2), R⁴ is methyl, R⁵ is —F, R⁶ is —OH, and R² is

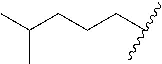 [SMILES: *CCCC(C)C]

optionally substituted with one, two, or three R^{20b}. In some embodiments, the compound is a compound of Formula (Ia″-2), R⁴ is methyl, R⁵ is —F, R⁶ is —OH, and R² is

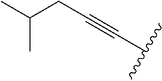 [SMILES: *C#CCC(C)C]

optionally substituted with one, two, or three R^{20b}. In some embodiments, the compound is a compound of Formula (Ia″-2), R⁴ is methyl, R⁵ is —F, R⁶ is —OH, and R² is

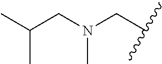 [SMILES: *CN(C)CC(C)C]

optionally substituted with one, two, or three R^{20b}.

In some embodiments, the compound is a compound of Formula (Ia″-2), R⁴ is methyl, R⁵ is —F, R⁶ is —OH, and R² is

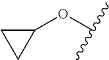 [SMILES: *OC1CC1]

optionally substituted with one, two, or three R^{20k}. In some embodiments, the compound is a compound of Formula (Ia″-2), R⁴ is methyl, R⁵ is —F, R⁶ is —OH, and R² is

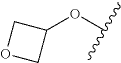 [SMILES: *OC1COC1]

optionally substituted with one, two, or three R^{20k}. In some embodiments, the compound is a compound of Formula (Ia″-2), R⁴ is methyl, R⁵ is —F, R⁶ is —OH, and R² is,

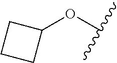 [SMILES: *OC1CCC1]

optionally substituted with one, two, or three R^{20k}. In some embodiments, the compound is a compound of Formula (Ia″-2), R⁴ is methyl, R⁵ is —F, R⁶ is —OH, and R² is

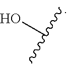 [SMILES: *O]

In some embodiments, the compound is a compound of Formula (Ia″-2), R⁴ is methyl, R⁵ is —F, R⁶ is —OH, and R² is

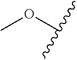 [SMILES: *OC]

optionally substituted with one, two, or three R^{20k}. In some embodiments, the compound is a compound of Formula (Ia″-2), R⁴ is methyl, R⁵ is —F, R⁶ is —OH, and R² is

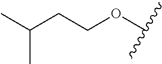 [SMILES: *OCCC(C)C]

optionally substituted with one, two, or three R^{20k}. In some embodiments, the compound is a compound of Formula (Ia″-2), R⁴ is methyl, R⁵ is —F, R⁶ is —OH, and R² is

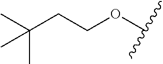 [SMILES: *OCCC(C)(C)C]

optionally substituted with one, two, or three R^{20k}. In some embodiments, the compound is a compound of Formula (Ia″-2), R⁴ is methyl, R⁵ is —F, R⁶ is —OH, and R² is

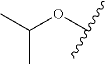 [SMILES: *OC(C)C]

optionally substituted with one, two, or three R^{20k}. In some embodiments, the compound is a compound of Formula (Ia″-2), R⁴ is methyl, R⁵ is —F, R⁶ is —OH, and R² is

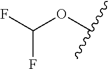 [SMILES: *OC(F)F]

optionally substituted with one R^{20k}. In some embodiments, the compound is a compound of Formula (Ia″-2), R⁴ is methyl, R⁵ is —F, R⁶ is —OH, and R² is

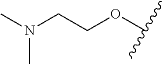 [SMILES: *OCCN(C)C]

optionally substituted with one, two, or three R^{20k}. In some embodiments, the compound is a compound of Formula (Ia″-2), R⁴ is methyl, R⁵ is —F, R⁶ is —OH, and R² is

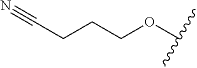 [SMILES: *OCCCC#N]

optionally substituted with one, two, or three R^{20k}. In some embodiments, the compound is a compound of Formula (Ia″-2), R⁴ is methyl, R⁵ is —F, R⁶ is —OH, and R² is

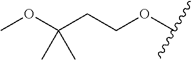 [SMILES: *OCCC(C)(C)OC]

optionally substituted with one, two, or three R^{20k}. In some embodiments, the compound is a compound of Formula (Ia″-2), R⁴ is methyl, R⁵ is —F, R⁶ is —OH, and R² is

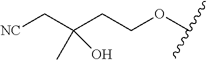 [SMILES: *OCCC(C)(O)CC#N]

optionally substituted with one, two, or three R^{20k}. In some embodiments, the compound is a compound of Formula (Ia″-2), R⁴ is methyl, R⁵ is —F, R⁶ is —OH, and R² is

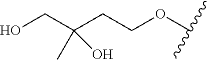 [SMILES: *OCCC(C)(O)CO]

optionally substituted with one, two, or three R^{20k}. In some embodiments, the compound is a compound of Formula (Ia″-2), R⁴ is methyl, R⁵ is —F, R⁶ is —OH, and R² is

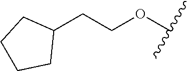 [SMILES: *OCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

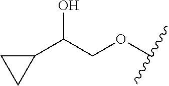 [SMILES: *OCC(O)C1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

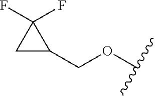 [SMILES: *OCC1CC1(F)F]

substituted with one, two, or three R^{20k}. In some embodiments, the compound is a compound of Formula (Ia″-2), R⁴ is methyl, R⁵ is —F, R⁶ is —OH, and R² is

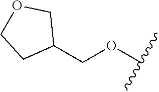 [SMILES: *OCC1CCOC1]

optionally substituted with one, two, or three R^{20k}. In some embodiments, the compound is a compound of Formula (Ia″-2), R⁴ is methyl, R⁵ is —F, R⁶ is —OH, and R² is

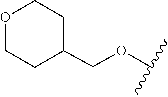 [SMILES: *OCC1CCOCC1]

optionally substituted with one, two, or three R^{20k}. In some embodiments, the compound is a compound of Formula (Ia″-2), R⁴ is methyl, R⁵ is —F, R⁶ is —OH, and R² is

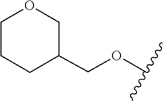 [SMILES: *OCC1CCCOC1]

optionally substituted with one, two, or three R^{20k} In some embodiments, the compound is a compound of Formula (Ia″-2), R⁴ is methyl, R⁵ is —F, R⁶ is —OH, and R² is

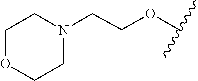 [SMILES: *OCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OCOR²⁵. In some embodiments, the compound is a compound of Formula (Ia″-2), R⁴ is methyl, R⁵ is —F, R⁶ is —OH, and R² is

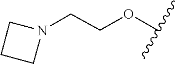 [SMILES: *OCCN1CCC1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

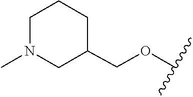 [SMILES: *OCC1CCCN(C)C1]

optionally substituted with one, two, or three R^{20k}. In some embodiments, the compound is a compound of Formula (Ia″-2), R⁴ is methyl, R⁵ is —F, R⁶ is —OH, and R² is

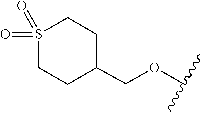 [SMILES: *OCC1CCS(=O)(=O)CC1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

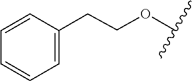 [SMILES: *OCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

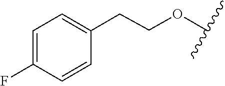 [SMILES: *OCCc1ccc(F)cc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

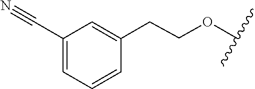 [SMILES: *OCCc1cccc(C#N)c1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is methyl, R⁵ is —F, R⁶ is —OH, and R² is

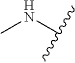 [SMILES: *NC]

optionally substituted with one, two, or three R^{20k}. In some embodiments, the compound is a compound of Formula (Ia″-2), R⁴ is methyl, R⁵ is —F, R⁶ is —OH, and R² is

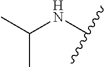 [SMILES: *NC(C)C]

optionally substituted with one, two, or three R^{20k}. In some embodiments, the compound is a compound of Formula (Ia″-2), R⁴ is methyl, R⁵ is —F, R⁶ is —OH, and R² is

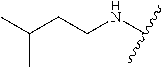 [SMILES: *NCCC(C)C]

optionally substituted with one, two, or three R^{20k}. In some embodiments, the compound is a compound of Formula (Ia″-2), R⁴ is methyl, R⁵ is —F, R⁶ is —OH, and R² is

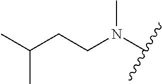 [SMILES: *N(C)CCC(C)C]

optionally substituted with one, two, or three R^{20k}. In some embodiments, the compound is a compound of Formula (Ia″-2), R⁴ is methyl, R⁵ is —F, R⁶ is —OH, and R² is

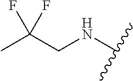 [SMILES: *NCC(C)(F)F]

optionally substituted with one, two, or three R^{20k}. In some embodiments, the compound is a compound of Formula (Ia″-2), R⁴ is methyl, R⁵ is —F, R⁶ is —OH, and R² is

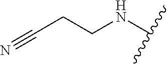 [SMILES: *NCCC#N]

optionally substituted with one, two, or three R^{20k}. In some embodiments, the compound is a compound of Formula (Ia″-2), R⁴ is methyl, R⁵ is —F, R⁶ is —OH, and R² is

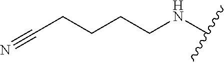 [SMILES: *NCCCCC#N]

optionally substituted with one, two, or three R^{20k}. In some embodiments, the compound is a compound of Formula (Ia″-2), R⁴ is methyl, R⁵ is —F, R⁶ is —OH, and R² is

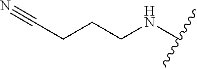 [SMILES: *NCCCC#N]

optionally substituted with one, two, or three R^{20k}. In some embodiments, the compound is a compound of Formula (Ia″-2), R⁴ is methyl, R⁵ is —F, R⁶ is —OH, and R² is

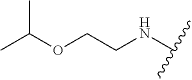 [SMILES: *NCCOC(C)C]

optionally substituted with one, two, or three R^{20k}. In some embodiments, the compound is a compound of Formula (Ia″-2), R⁴ is methyl, R⁵ is —F, R⁶ is —OH, and R² is

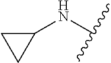 [SMILES: *NC1CC1]

optionally substituted with one, two, or three R^{20k}. In some embodiments, the compound is a compound of Formula (Ia″-2), R⁴ is methyl, R⁵ is —F, R⁶ is —OH, and R² is

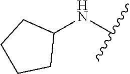 [SMILES: *NC1CCCC1]

optionally substituted with one, two, or three R^{20k}. In some embodiments, the compound is a compound of Formula (Ia″-2), R⁴ is methyl, R⁵ is —F, R⁶ is —OH, and R² is

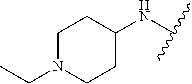 [SMILES: *NC1CCN(CC)CC1]

optionally substituted with one, two, or three R^{20k}. In some embodiments, the compound is a compound of Formula (Ia″-2), R⁴ is methyl, R⁵ is —F, R⁶ is —OH, and R² is

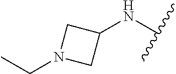 [SMILES: *NC1CN(CC)C1]

optionally substituted with one, two, or three R^{20k}. In some embodiments, the compound is a compound of Formula (Ia″-2), R⁴ is methyl, R⁵ is —F, R⁶ is —OH, and R² is

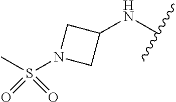 [SMILES: *NC1CN(S(C)(=O)=O)C1]

optionally substituted with one, two, or three R^{20k}. In some embodiments, the compound is a compound of Formula (Ia″-2), R⁴ is methyl, R⁵ is —F, R⁶ is —OH, and R² is

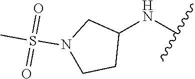 [SMILES: *NC1CCN(S(C)(=O)=O)C1]

optionally substituted with one, two, or three R^{20k}. In some embodiments, the compound is a compound of Formula (Ia″-2), R⁴ is methyl, R⁵ is —F, R⁶ is —OH, and R² is

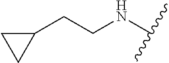 [SMILES: *NCCC1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

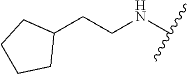 [SMILES: *NCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

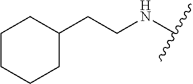 [SMILES: *NCCC1CCCCC1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

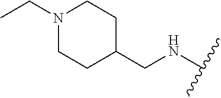 [SMILES: *NCC1CCN(CC)CC1]

optionally substituted with one, two, or three R^{20k}. In some embodiments, the compound is a compound of Formula (Ia″-2), R⁴ is methyl, R⁵ is —F, R⁶ is —OH, and R² is

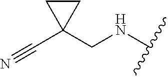 [SMILES: *NCC1(C#N)CC1]

optionally substituted with one, two, or three R^{20k}. In some embodiments, the compound is a compound of Formula (Ia″-2), R⁴ is methyl, R⁵ is —F, R⁶ is —OH, and R² is

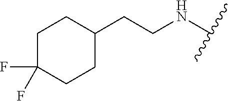 [SMILES: *NCCC1CCC(F)(F)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

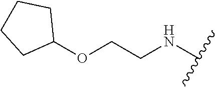 [SMILES: *NCCOC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

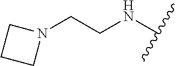 [SMILES: *NCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

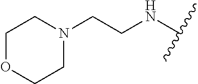 [SMILES: *NCCN1CCOCC1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

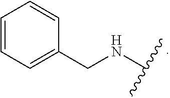 [SMILES: *NCc1ccccc1]

In some embodiments, the compound is a compound of Formula (Ia″-2), R⁴ is methyl, R⁵ is —F, R⁶ is —OH, and R² is

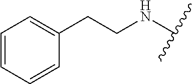 [SMILES: *NCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

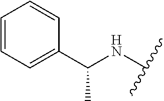 [SMILES: *N[C@H](C)c1ccccc1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

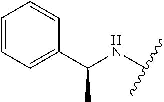 [SMILES: *N[C@@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

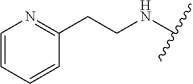 [SMILES: *NCCc1ccccn1]

optionally substituted with one, two, or three R^{20k} and optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is methyl, R⁵ is —F, R⁶ is —OH, and R² is

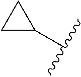 [SMILES: *C1CC1]

optionally substituted with one, two, or three R^{20b}. In some embodiments, the compound is a compound of Formula (Ia″-2), R⁴ is methyl, R⁵ is —F, R⁶ is —OH, and R² is

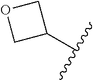 [SMILES: *C1COC1]

optionally substituted with one, two, or three R^{20b}. In some embodiments, the compound is a compound of Formula (Ia″-2), R⁴ is methyl, R⁵ is —F, R⁶ is —OH, and R² is

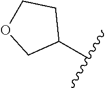 [SMILES: *C1CCOC1]

optionally substituted with one, two, or three R^{20b}. In some embodiments, the compound is a compound of Formula (Ia″-2), R⁴ is methyl, R⁵ is —F, R⁶ is —OH, and R² is

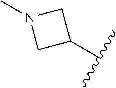 [SMILES: *C1CN(C)C1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

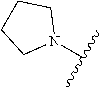 [SMILES: *N1CCCC1]

optionally substituted with one, two, or three R^{20b}. In some embodiments, the compound is a compound of Formula (Ia″-2), R⁴ is methyl, R⁵ is —F, R⁶ is —OH, and R² is

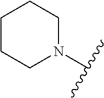 [SMILES: *N1CCCCC1]

optionally substituted with one, two, or three R^{20b}. In some embodiments, the compound is a compound of Formula (Ia″-2), R⁴ is methyl, R⁵ is —F, R⁶ is —OH, and R² is

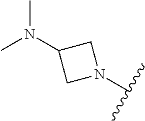 [SMILES: *N1CC(N(C)C)C1]

optionally substituted with one, two, or three R^{20b}. In some embodiments, the compound is a compound of Formula (Ia″-2), R⁴ is methyl, R⁵ is —F, R⁶ is —OH, and R² is

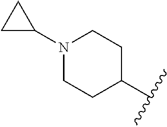 [SMILES: *C1CCN(C2CC2)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

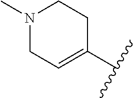 [SMILES: *C1=CCN(C)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R² is

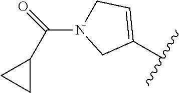 [SMILES: *C1=CCN(C(=O)C2CC2)C1]

optionally substituted with one, two, or three R^{20b}. In some embodiments, the compound is a compound of Formula (Ia″-2), R⁴ is methyl, R⁵ is —F, R⁶ is —OH, and R² is

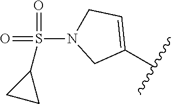 [SMILES: *C1=CCN(S(=O)(=O)C2CC2)C1]

optionally substituted with one, two, or three R^{20b}. In some embodiments, the compound is a compound of Formula (Ia″-2), R⁴ is methyl, R⁵ is —F, R⁶ is —OH, and R² is

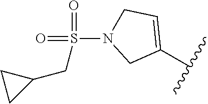 [SMILES: *C1=CCN(S(=O)(=O)CC2CC2)C1]

optionally substituted with one, two, or three R^{20b}. In some embodiments, the compound is a compound of Formula (Ia″-2), R⁴ is methyl, R⁵ is —F, R⁶ is —OH, and R² is

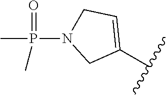 [SMILES: *C1=CCN(P(C)(C)=O)C1]

optionally substituted with one, two, or three R^{20b}. In some embodiments, the compound is a compound of Formula (Ia″-2), R⁴ is methyl, R⁵ is —F, R⁶ is —OH, and R² is

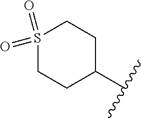 [SMILES: *C1CCS(=O)(=O)CC1]

optionally substituted with one, two, or three R^{20b}. In some embodiments, the compound is a compound of Formula (Ia″-2), R⁴ is methyl, R⁵ is —F, R⁶ is —OH, and R² is

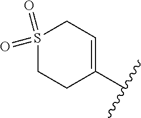 [SMILES: *C1=CCS(=O)(=O)CC1]

optionally substituted with one, two, or three R^{20b}. In some embodiments, the compound is a compound of Formula (Ia″-2), R⁴ is methyl, R⁵ is —F, R⁶ is —OH, and R² is

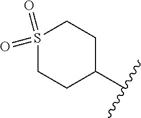 [SMILES: *C1CCS(=O)(=O)CC1]

optionally substituted with one, two, or three R^{20b}. In some embodiments, the compound is a compound of Formula (Ia″-2), R⁴ is methyl, R⁵ is —F, R⁶ is —OH, and R² is

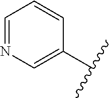 [SMILES: *c1cccnc1]

optionally substituted with one, two, or three R^{20b}. In some embodiments, the compound is a compound of Formula (Ia″-2), R⁴ is methyl, R⁵ is —F, R⁶ is —OH, and R² is

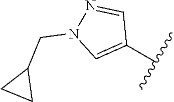 [SMILES: *c1cnn(CC2CC2)c1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is methyl, R⁵ is —F, R⁶ is —OH, and R² is independently hydrogen. In some embodiments, the compound is a compound of Formula (Ia″-2), R⁴ is methyl, R⁵ is —F, R⁶ is —OH, and R² is independently halogen. In some embodiments, the compound is a compound of Formula (Ia″-2), R⁴ is methyl, R⁵ is —F, R⁶ is —OH, and R² is independently —CN. In some embodiments, the compound is a compound of Formula (Ia″-2), R⁴ is methyl, R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments, the compound is a compound of Formula (Ia″-2), R⁴ is methyl, R⁵ is —F, R⁶ is —OH, and R² is independently —OR¹². In some embodiments, the compound is a compound of Formula (Ia″-2), R⁴ is methyl, R⁵ is —F, R⁶ is —OH, and R² is independently —SR¹². In some embodiments, the compound is a compound of Formula (Ia″-2), R⁴ is methyl, R⁵ is —F, R⁶ is —OH, and R² is independently —N(R¹²)(R¹³). In some embodiments, the compound is a compound of Formula (Ia″-2), R⁴ is methyl, R⁵ is —F, R⁶ is —OH, and R² is independently —C(O)OR¹². In some embodiments, the compound is a compound of Formula (Ia″-2), R⁴ is methyl, R⁵ is —F, R⁶ is —OH, and R² is independently —OC(O)N(R¹²)(R¹³). In some embodiments, the compound is a compound of Formula (Ia″-2), R⁴ is methyl, R⁵ is —F, R⁶ is —OH, and R² is independently —N(R¹⁴)C(O)N(R¹²)(R¹³). In some embodiments, the compound is a compound of Formula (Ia″-2), R⁴ is methyl, R⁵ is —F, R⁶ is —OH, and R² is independently —N(R¹⁴)C(O)OR¹². In some embodiments, the compound is a compound of Formula (Ia″-2), R⁴ is methyl, R⁵ is —F, R⁶ is —OH, and R² is independently —N(R¹⁴)S(O)₂R¹⁵. In some embodiments, the compound is a compound of Formula (Ia″-2), R⁴ is methyl, R⁵ is —F, R⁶ is —OH, and R² is independently —C(O)R¹⁵. In some embodiments, the compound is a compound of Formula (Ia″-2), R⁴ is methyl, R⁵ is —F, R⁶ is —OH, and R² is independently —S(O)R¹⁵. In some embodiments, the compound is a compound of Formula (Ia″-2), R⁴ is methyl, R⁵ is —F, R⁶ is —OH, and R² is independently —OC(O)R¹⁵. In some embodiments, the compound is a compound of Formula (Ia″-2), R⁴ is methyl, R⁵ is —F, R⁶ is —OH, and R² is independently —C(O)N(R¹²)(R¹³). In some embodiments, the compound is a compound of Formula (Ia″-2), R⁴ is methyl, R⁵ is —F, R⁶ is —OH, and R² is independently —C(O)C(O)N(R¹²)(R¹³). In some embodiments, the compound is a compound of Formula (Ia″-2), R⁴ is methyl, R⁵ is —F, R⁶ is —OH, and R² is independently —N(R¹⁴)C(O)R¹⁵. In some embodiments, the compound is a compound of Formula (Ia″-2), R⁴ is methyl, R⁵ is —F, R⁶ is —OH, and R² is independently —S(O)₂R¹⁵. In some embodiments, the compound is a compound of Formula (Ia″-2), R⁴ is methyl, R⁵ is —F, R⁶ is —OH, and R² is independently —S(O)₂N(R¹²)(R¹³). In some embodiments, the compound is a compound of Formula (Ia″-2), R⁴ is methyl, R⁵ is —F, R⁶ is —OH, and R² is independently —S(═O)(═NH)N(R¹²)(R¹³). In some embodiments, the compound is a compound of Formula (Ia″-2), R⁴ is methyl, R⁵ is —F, R⁶ is —OH, and R² is independently —CH₂C(O)N(R¹²)(R¹³). In some embodiments, the compound is a compound of Formula (Ia″-2), R⁴ is methyl, R⁵ is —F, R⁶ is —OH, and R² is independently —CH₂N(R¹⁴)C(O)R¹⁵. In some embodiments, the compound is a compound of Formula (Ia″-2), R⁴ is methyl, R⁵ is —F, R⁶ is —OH, and R² is independently —CH₂S(O)₂R¹⁵. In some embodiments, the compound is a compound of Formula (Ia″-2), R⁴ is methyl, R⁵ is —F, R⁶ is —OH, and R² is independently —CH₂S(O)₂N(R¹²)(R¹³). In some embodiments, the compound is a compound of Formula (Ia″-2), R⁴ is methyl, R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20b}. In some embodiments, the compound is a compound of Formula (Ia″-2), R⁴ is methyl, R⁵ is —F, R⁶ is —OH, and R² is independently C₁₋₆ alkyl substituted with one, two, or three R^{20b}. In some embodiments, the compound is a compound of Formula (Ia″-2), R⁴ is methyl, R⁵ is —F, R⁶ is —OH, and R² is independently C₂₋₆alkenyl substituted with one, two, or three R^{20b}. In some embodiments, the compound is a compound of Formula (Ia″-2), R⁴ is methyl, R⁵ is —F, R⁶ is —OH, and R² is independently C₂₋₆alkynyl substituted with one, two, or three R^{20b}. In some embodiments, the compound is a compound of Formula (Ia″-2), R⁴ is methyl, R⁵ is —F, R⁶ is —OH, and R² is independently C₃₋₆cycloalkyl substituted with one, two, or three R^{20b}. In some embodiments, the compound is a compound of Formula (Ia″-2), R⁴ is methyl, R⁵ is —F, R⁶ is —OH, and R² is independently C₂₋₉heterocycloalkyl substituted with one, two, or three R^{20b}. In some embodiments, the compound is a compound of Formula (Ia″-2), R⁴ is methyl, R⁵ is —F, R⁶ is —OH, and R² is independently C₆₋₁₀aryl substituted with one, two, or three R^{20b}. In some embodiments, the compound is a compound of Formula (Ia″-2), R⁴ is methyl, R⁵ is —F, R⁶ is —OH, and R² is independently C₁₋₉heteroaryl substituted with one, two, or three R^{20b}. In some embodiments, the compound is a compound of Formula (Ia″-2), R⁴ is methyl, R⁵ is —F, R⁶ is —OH, and R² is independently C₁₋₆alkyl. In some embodiments, the compound is a compound of Formula (Ia″-2), R⁴ is methyl, R⁵ is —F, R⁶ is —OH, and R² is independently C₂₋₆alkenyl. In some embodiments, the compound is a compound of Formula (Ia″-2), R⁴ is methyl, R⁵ is —F, R⁶ is —OH, and R² is independently C₂₋₆alkynyl. In some embodiments, the compound is a compound of Formula (Ia″-2), R⁴ is methyl, R⁵ is —F, R⁶ is —OH, and R is independently C₃₋₆cycloalkyl. In some embodiments, the compound is a compound of Formula (Ia″-2), R⁴ is methyl, R⁵ is —F, R⁶ is —OH, and R² is independently C₂₋₉heterocycloalkyl. In some embodiments, the compound is a compound of Formula (Ia″-2), R⁴ is methyl, R⁵ is —F, R⁶ is —OH, and R² is independently C₆₋₁₀aryl. In some embodiments, the compound is a compound of Formula (Ia″-2), R⁴ is methyl, R⁵ is —F, R⁶ is —OH, and R² is independently C₁₋₉heteroaryl. In some embodiments, the compound is a compound of Formula (Ia″-2), R⁴ is methyl, R⁵ is —F, R⁶ is —OH, and R² is independently C₁alkyl. In some embodiments, the compound is a compound of Formula (Ia″-2), R⁴ is methyl, R⁵ is —F, R⁶ is —OH, and R² is independently C₂alkyl. In some embodiments, the compound is a compound of Formula (Ia″-2), R⁴ is methyl, R⁵ is —F, R⁶ is —OH, and R² is independently C₃alkyl. In some embodiments, the compound is a compound of Formula (Ia″-2), R⁴ is methyl, R⁵ is —F, R⁶ is —OH, and R² is independently C₄alkyl. In some embodiments, the compound is a compound of Formula (Ia″-2), R⁴ is methyl, R⁵ is —F, R⁶ is —OH, and R² is independently C₅alkyl. In some embodiments, the compound is a compound of Formula (Ia″-2), R⁴ is methyl, R⁵ is —F, R⁶ is —OH, and R² is independently C₆alkyl.

In some embodiments, the compound is a compound of Formula (Ia″-2), R⁴ is methyl, R⁵ is —F, R⁶ is —OH, and R² is independently —OH. In some embodiments, the compound is a compound of Formula (Ia″-2), R⁴ is methyl, R⁵ is —F, R⁶ is —OH, and R² is independently —SH. In some embodiments, the compound is a compound of Formula (Ia″-2), R⁴ is methyl, R⁵ is —F, R⁶ is —OH, and R² is independently —NH₂. In some embodiments, the compound is a compound of Formula (Ia″-2), R⁴ is methyl, R⁵ is —F, R⁶ is —OH, and R² is independently —C(O)OH. In some embodiments, the compound is a compound of Formula (Ia″-2), R⁴ is methyl, R⁵ is —F, R⁶ is —OH, and R² is independently —OC(O)NH₂. In some embodiments, the compound is a compound of Formula (Ia″-2), R⁴ is methyl, R⁵ is —F, R⁶ is —OH, and R² is independently —NHC(O)NH₂. In some embodiments, the compound is a compound of Formula (Ia″-2), R⁴ is methyl, R⁵ is —F, R⁶ is —OH, and R² is independently —NHC(O)OH. In some embodiments, the compound is a compound of Formula (Ia″-2), R⁴ is methyl, R⁵ is —F, R⁶ is —OH, and R² is independently —NHS(O)₂R¹⁵. In some embodiments, the compound is a compound of Formula (Ia″-2), R⁴ is methyl, R⁵ is —F, R⁶ is —OH, and R² is independently —C(O)R¹⁵. In some embodiments, the compound is a compound of Formula (Ia″-2), R⁴ is methyl, R⁵ is —F, R⁶ is —OH, and R² is independently —S(O)R¹⁵. In some embodiments, the compound is a compound of Formula (Ia″-2), R⁴ is methyl, R⁵ is —F, R⁶ is —OH, and R² is independently —OC(O)R¹⁵. In some embodiments, the compound is a compound of Formula (Ia″-2), R⁴ is methyl, R⁵ is —F, R⁶ is —OH, and R² is independently —OC(O)CH₃. In some embodiments, the compound is a compound of Formula (Ia″-2), R⁴ is methyl, R⁵ is —F, R⁶ is —OH, and R² is independently —C(O)NH₂. In some embodiments, the compound is a compound of Formula (Ia″-2), R⁴ is methyl, R⁵ is —F, R⁶ is —OH, and R² is independently —C(O)C(O)NH₂. In some embodiments, the compound is a compound of Formula (Ia″-2), R⁴ is methyl, R⁵ is —F, R⁶ is —OH, and R² is independently —NHC(O)R¹⁵. In some embodiments, the compound is a compound of Formula (Ia″-2), R⁴ is methyl, R⁵ is —F, R⁶ is —OH, and R² is independently —S(O)₂R¹⁵. In some embodiments, the compound is a compound of Formula (Ia″-2), R⁴ is methyl, R⁵ is —F, R⁶ is —OH, and R² is independently —S(O)₂N(R¹²)(R¹³). In some embodiments, the compound is a compound of Formula (Ia″-2), R⁴ is methyl, R⁵ is —F, R⁶ is —OH, and R² is independently —S(═O)(═NH)NH₂. In some embodiments, the compound is a compound of Formula (Ia″-2), R⁴ is methyl, R⁵ is —F, R⁶ is —OH, and R² is independently —CH₂C(O)NH₂. In some embodiments, the compound is a compound of Formula (Ia″-2), R⁴ is methyl, R⁵ is —F, R⁶ is —OH, and R² is independently —CH₂NHC(O)R¹⁵. In some embodiments, the compound is a compound of Formula (Ia″-2), R⁴ is methyl, R⁵ is —F, R⁶ is —OH, and R² is independently —CH₂S(O)₂R¹⁵. In some embodiments, the compound is a compound of Formula (Ia″-2), R⁴ is methyl, R⁵ is —F, R⁶ is —OH, and R² is independently —CH₂S(O)₂NH₂.

In some embodiments, the compound is a compound of Formula (Ia″-2), R⁴ is methyl, R⁵ is —F, R⁶ is —OH, and R² is independently —NH(R¹²). In some embodiments, the compound is a compound of Formula (Ia″-2), R⁴ is methyl, R⁵ is —F, R⁶ is —OH, and R² is independently —OC(O)N(R¹²)(R¹³). In some embodiments, the compound is a compound of Formula (Ia″-2), R⁴ is methyl, R⁵ is —F, R⁶ is —OH, and R² is independently —NHC(O)N(R¹²). In some embodiments, the compound is a compound of Formula (Ia″-2), R⁴ is methyl, R⁵ is —F, R⁶ is —OH, and R² is independently —NHC(O)OR¹². In some embodiments, the compound is a compound of Formula (Ia″-2), R⁴ is methyl, R⁵ is —F, R⁶ is —OH, and R² is independently —C(O)N(R¹²)(R¹³). In some embodiments, the compound is a compound of Formula (Ia″-2), R⁴ is methyl, R⁵ is —F, R⁶ is —OH, and R² is independently —C(O)C(O)NH(R¹²). In some embodiments, the compound is a compound of Formula (Ia″-2), R⁴ is methyl, R⁵ is —F, R⁶ is —OH, and R² is independently —S(O)₂NH(R¹²). In some embodiments, the compound is a compound of Formula (Ia″-2), R⁴ is methyl, R⁵ is —F, R⁶ is —OH, and R² is independently —S(═O)(═NH)NH(R¹²). In some embodiments, the compound is a compound of Formula (Ia″-2), R⁴ is methyl, R⁵ is —F, R⁶ is —OH, and R² is independently —CH₂C(O)NH(R¹²). In some embodiments, the compound is a compound of Formula (Ia″-2), R⁴ is methyl, R⁵ is —F, R⁶ is —OH, and R² is independently —CH₂S(O)₂NH(R¹²).

In some embodiments, the compound is a compound of Formula (Ia″-2), R⁴ is methyl, R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In some embodiments, the compound is a compound of Formula (Ia″-2), R⁴ is methyl, R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}.

In some embodiments, the compound is a compound of Formula (Ia″-2), R⁴ is methyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

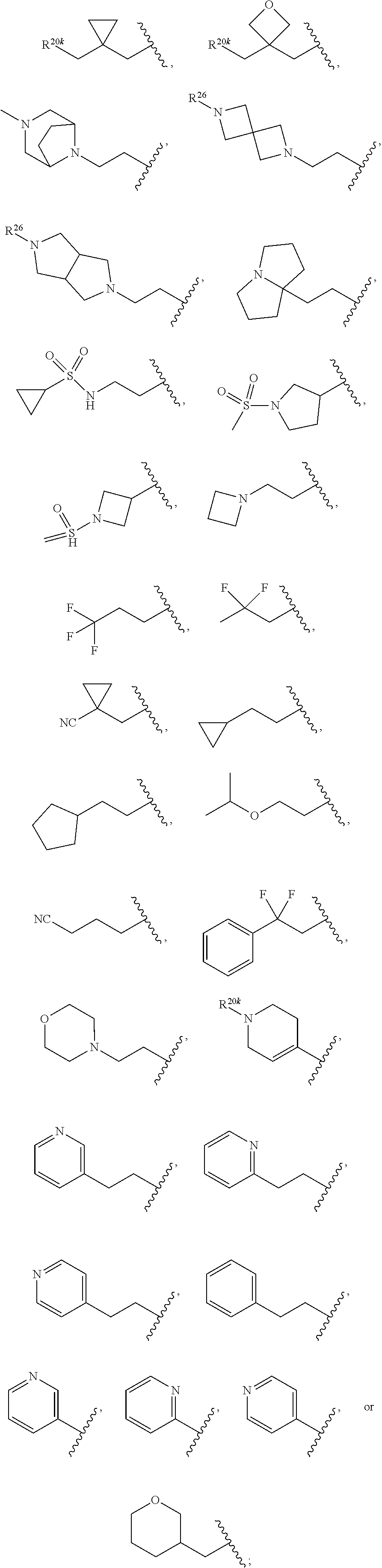

R¹⁵ is independently

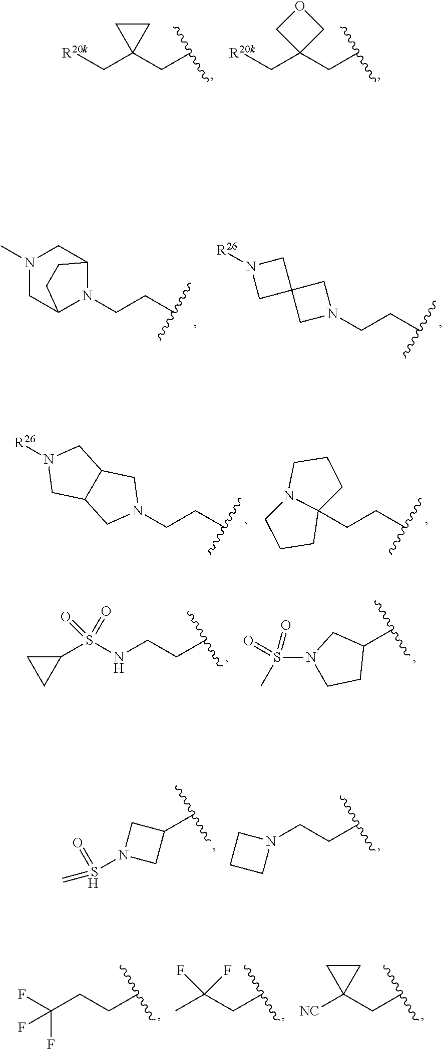

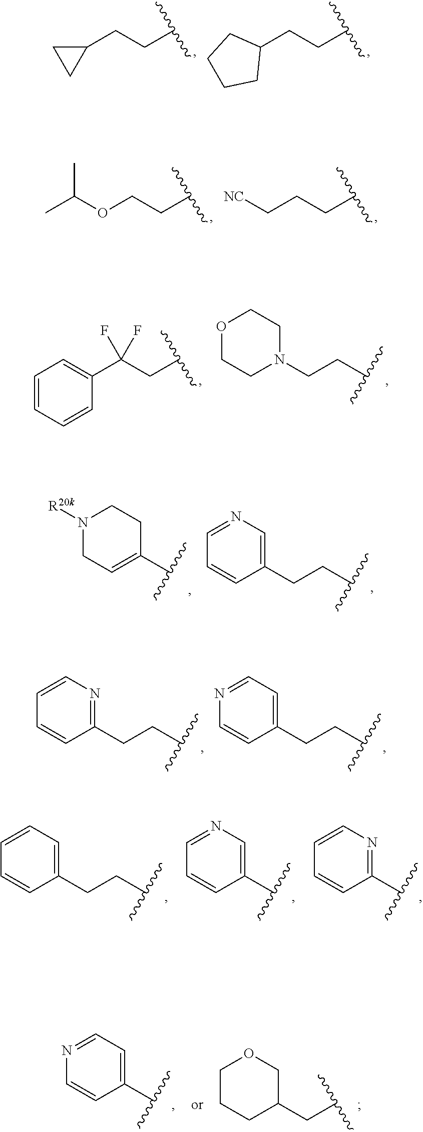

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is methyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

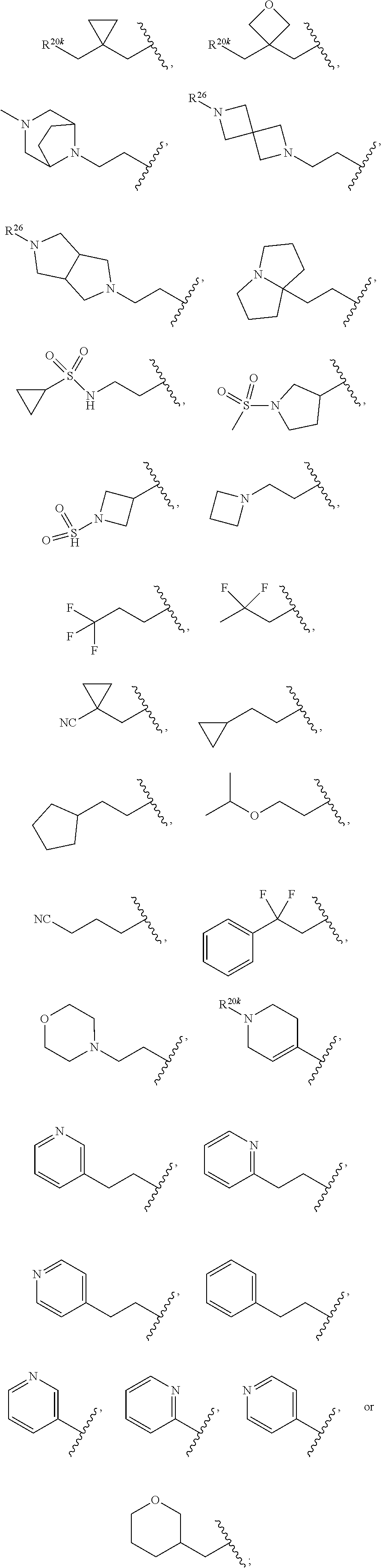

R¹⁵ is independently

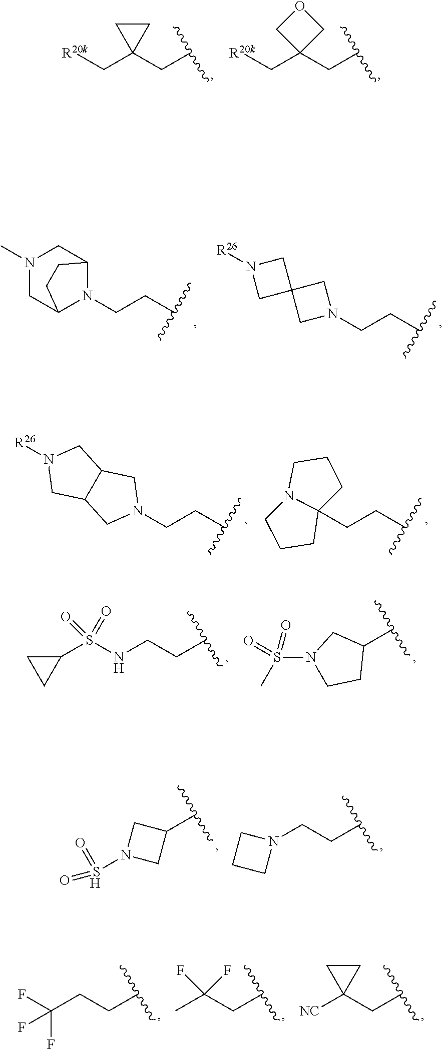

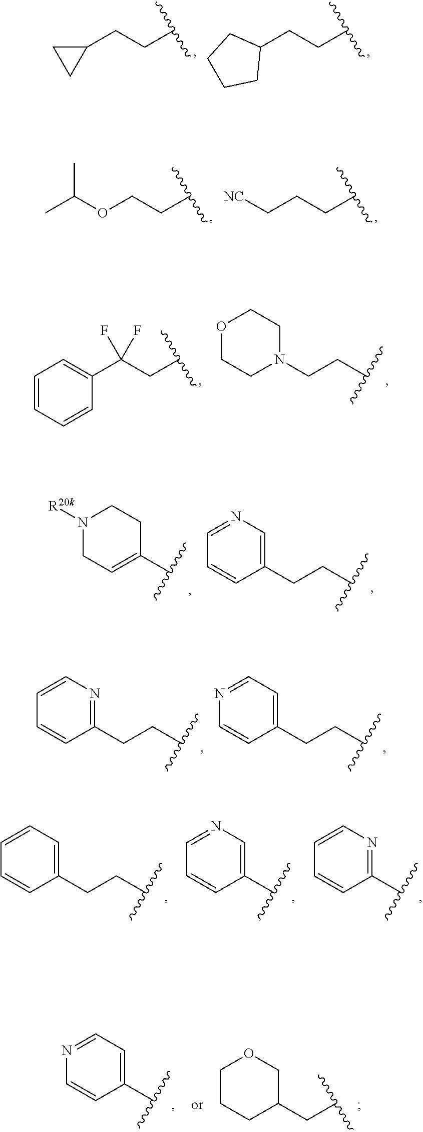

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is methyl, R⁵ is —F, R⁶ is —OH, and R² is selected from

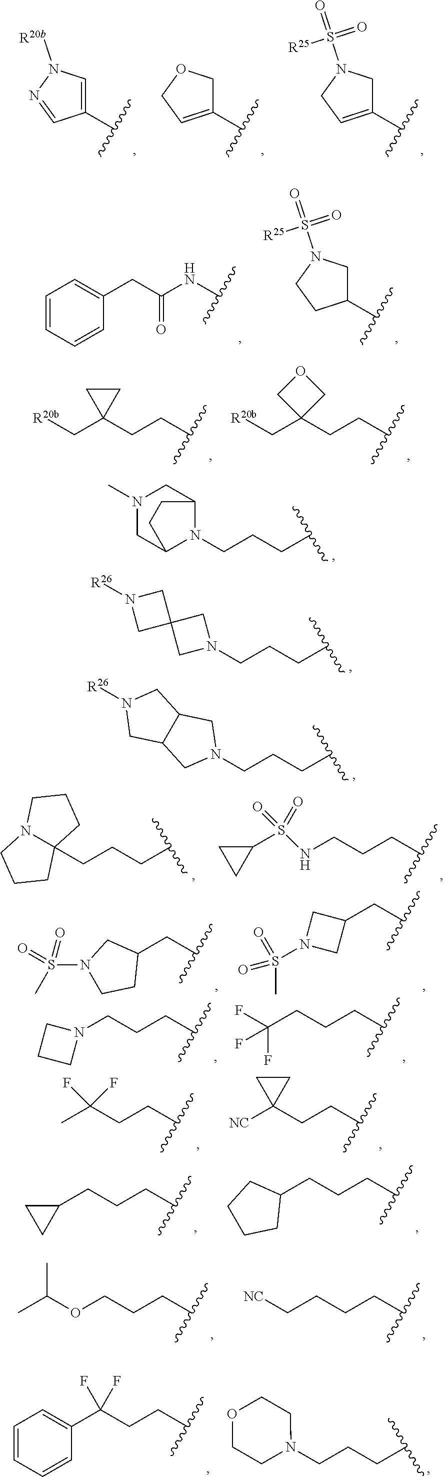

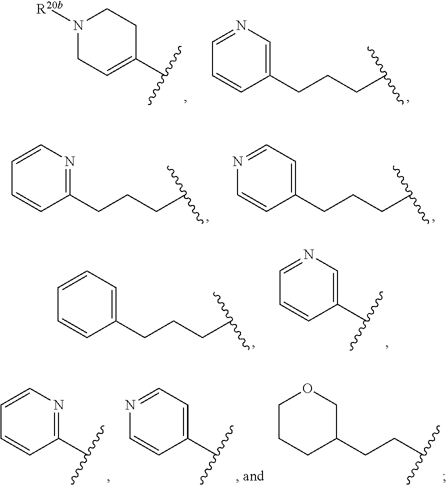

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is methyl, R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is methyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² and R¹⁵ are independently

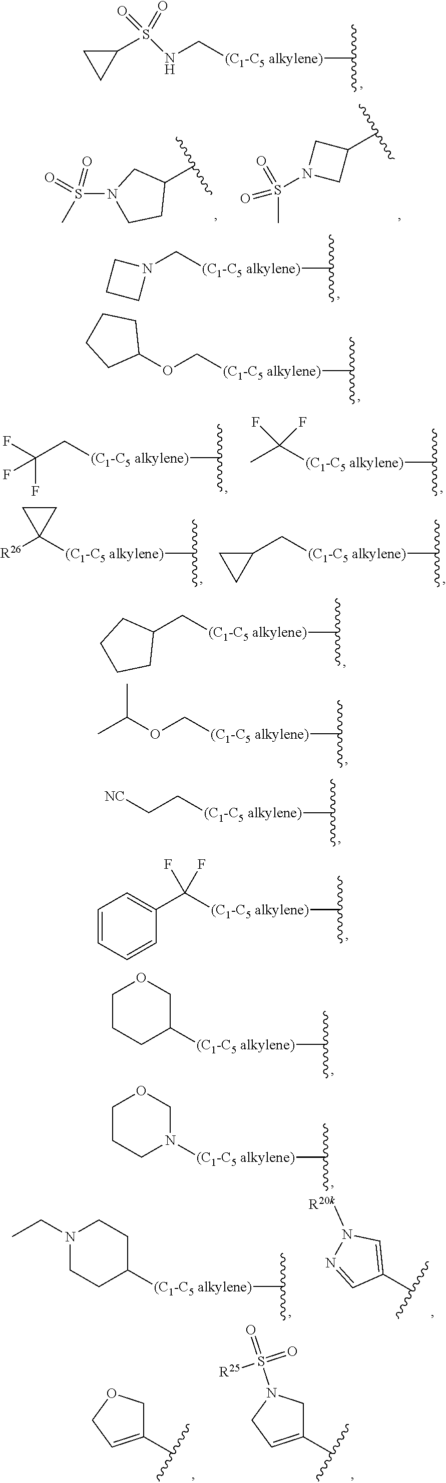

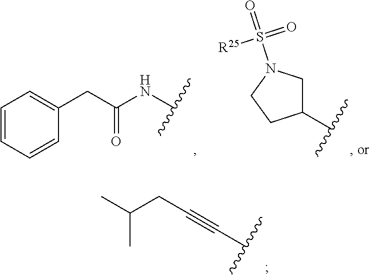

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is methyl, R⁵ is —F, R⁶ is —OH, and R² is independently

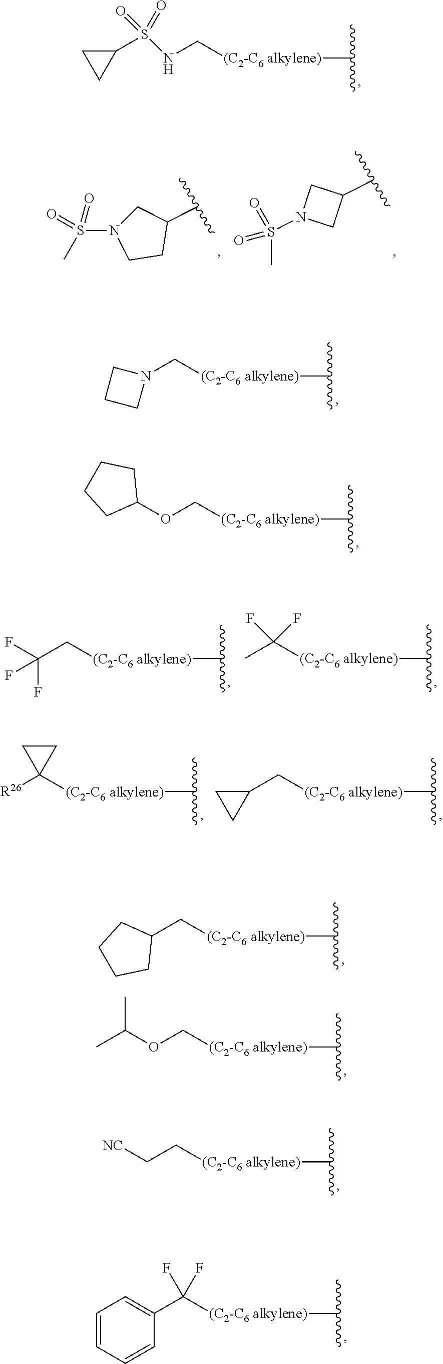

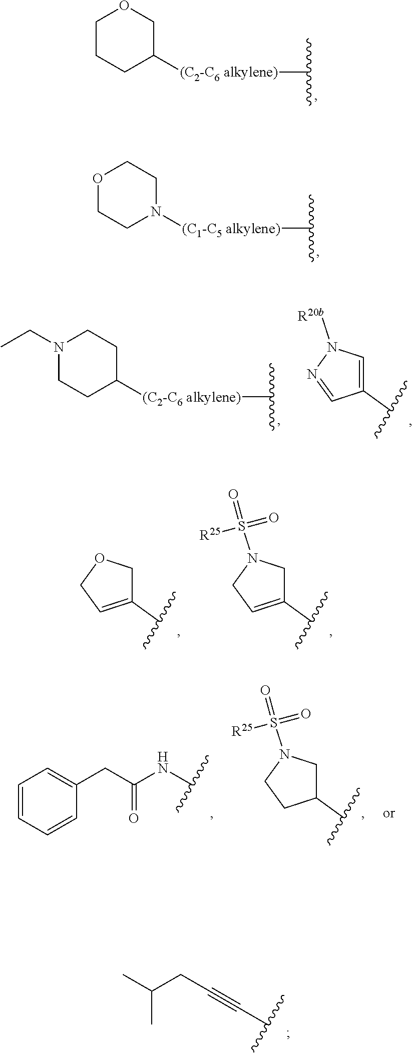

and R²⁶ is independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is cyclopropyl, R⁵ is —F, R⁶ is —OH, and R² is C₁₋₆ alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₁₋₅alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₁₋₄alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₁₋₃alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₁₋₂alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₁alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₂alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₃alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₄alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₅alkyl, optionally substituted with one, two, or three R^{20b}. In some embodiments, the compound is a compound of Formula (Ia″-2), R⁴ is cyclopropyl, R⁵ is —F, R⁶ is —OH, and R² is C₆alkyl, optionally substituted with one, two, or three R^{20b}. In some embodiments, the compound is a compound of Formula (Ia″-2), R⁴ is cyclopropyl, R⁵ is —F, R⁶ is —OH, and each R^{20b} is independently selected from —N(R²²)(R²³), R²² is C₁₋₆alkyl, and R²³ is C₁₋₆alkyl. In some embodiments, the compound is a compound of Formula (Ia″-2), R⁴ is cyclopropyl, R⁵ is —F, R⁶ is —OH, and R² is C₂₋₆alkynyl, optionally substituted with one, two, or three R^{20b}. In some embodiments, the compound is a compound of Formula (Ia″-2), R⁴ is cyclopropyl, R⁵ is —F, R⁶ is —OH, and R² is selected from any group listed in Table 2

In some embodiments, the compound is a compound of Formula (Ia″-2), R⁴ is cyclopropyl, R⁵ is —F, R⁶ is —OH, and R² is —OR¹². In some embodiments, the compound is a compound of Formula (Ia″-2), R⁴ is cyclopropyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}. In some embodiments, the compound is a compound of Formula (Ia″-2), R⁴ is cyclopropyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆ heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is —OR¹² and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}; and each R^{20k} is independently selected from oxo, —CN, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆ alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆alkyl. In some embodiments, the compound is a compound of Formula (Ia″-2), R⁴ is cyclopropyl, R⁵ is —F, R⁶ is —OH, and R² is —OR¹² and R¹² is independently selected from C₁₋₆alkyl, C₃₄cycloalkyl, C₃₋₅heterocycloalkyl, and —CH₂—C₄₋₅heterocycloalkyl, wherein C₁₋₆alkyl, C₃₋₄cycloalkyl, C₃₋₅heterocycloalkyl, and —CH₂—C₄₋₅heterocycloalkyl, are optionally substituted with one, two, or three R^{20k}; and each R^{20k} is independently selected from oxo, halogen, —CN, C₁₋₆alkyl, C₃₋₆cycloalkyl, C₂₋₉heterocycloalkyl, C₆₋₁₀aryl, C₁₋₉heteroaryl, —OR²¹, and —N(R²²)(R²³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups independently selected from halogen, oxo, —CN, C₁₋₆alkyl, and —OR²¹; R²¹ is H; R²² is C₁₋₆alkyl, and R²³ is C₁₋₆ alkyl. In some embodiments, the compound is a compound of Formula (Ia″-2), R⁴ is cyclopropyl, R⁵ is —F, R⁶ is —OH, and R² is a group selected from any one of the groups listed in Table 2

In some embodiments, the compound is a compound of Formula (Ia″-2), R⁴ is cyclopropyl, R⁵ is —F, R⁶ is —OH, and R² is —N(R¹²)(R¹³). In some embodiments, the compound is a compound of Formula (Ia″-2), R⁴ is cyclopropyl, R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}. In some embodiments, the compound is a compound of Formula (Ia″-2), R⁴ is cyclopropyl, R⁵ is —F, R⁶ is —OH, and R² is —N(R¹²)(R¹³), R¹³ is hydrogen; R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}; each R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, and —S(O)₂R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen; R²¹ is C₁₋₆alkyl; and R²⁵ is C₁₋₆alkyl. In some embodiments, the compound is a compound of Formula (Ia″-2), R⁴ is cyclopropyl, R⁵ is —F, R⁶ is —OH, and R² is a group selected from any groups listed in Table 2.

In some embodiments, the compound is a compound of Formula (Ia″-2), R⁴ is cyclopropyl, R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}. In some embodiments, the compound is a compound of Formula (Ia″-2), R⁴ is cyclopropyl, R⁵ is —F, R⁶ is —OH, and R² is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is selected from C₃₋₆cycloalkyl, C₂₋₉heterocycloalkyl, C₆₋₁₀aryl, and C₁₋₉ heteroaryl, wherein C₃₋₆cycloalkyl, C₂₋₉heterocycloalkyl, C₆₋₁₀aryl, and C₁₋₉heteroaryl are optionally substituted with one, two, or three R^{20b}; each R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂; each R²² is independently C₁₋₆alkyl; each R²³ is C₁₋₆alkyl; and each R²⁵ is selected from C₁₋₆alkyl, C₃₋₆cycloalkyl, and —CH₂—C₃₋₆cycloalkyl. In some embodiments, the compound is a compound of Formula (Ia″-2), R⁴ is cyclopropyl, R⁵ is —F, R⁶ is —OH, and R² is a group selected from Table 2.\

In some embodiments, the compound is a compound of Formula (Ia″-2), R⁴ is cyclopropyl, R⁵ is —F, R⁶ is —OH, and R² is independently selected from —C(O)OR¹² and —C(O)N(R¹²)(R¹³). In some embodiments, the compound is a compound of Formula (Ia″-2), R⁴ is cyclopropyl, R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia″-2), R⁴ is cyclopropyl, R⁵ is —F, R⁶ is —OH, and R³ is independently selected from —C(O)OR¹² and —C(O)N(R¹²)(R¹³). In some embodiments, the compound is a compound of Formula (Ia″-2), R⁴ is cyclopropyl, R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one, two, or three R^{20k}; R^{20k} is independently selected from oxo, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, wherein C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R³ is independently selected from —C(O)OR¹² and —C(O)N(R¹²)(R¹³); R¹³ is hydrogen; R¹² is C₁₋₆alkyl optionally substituted with one C₆₋₁₀aryl. In some embodiments, the compound is a compound of Formula (Ia″-2), R⁴ is cyclopropyl, R⁵ is —F, R⁶ is —OH, and R² is a group selected from Table 2.

In some embodiments, the compound is a compound of Formula (Ia″-2), R⁴ is cycloproyl, R⁵ is —F, R⁶ is —OH, and R² is selected from

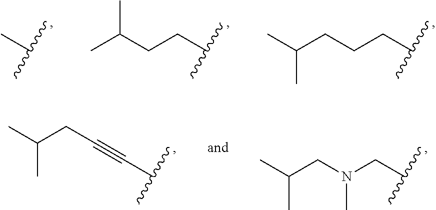

each of which is optionally substituted with one, two, or three R^{20b}. In some embodiments, the compound is a compound of Formula (Ia″-2), R⁴ is cyclopropyl, R⁵ is —F, R⁶ is —OH, and R² is

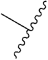

optionally substituted with one, two, or three R^{2b}. In some embodiments, the compound is a compound of Formula (Ia″-2), R⁴ is cyclopropyl, R⁵ is —F, R⁶ is —OH, and R² is

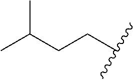 [SMILES: *CCC(C)C]

optionally substituted with one, two, or three R^{20b}. In some embodiments, the compound is a compound of Formula (Ia″-2), R⁴ is cyclopropyl, R⁵ is —F, R⁶ is —OH, and R² is

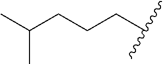 [SMILES: *CCCC(C)C]

optionally substituted with one, two, or three R^{20b}. In some embodiments, the compound is a compound of Formula (Ia″-2), R⁴ is cyclopropyl, R⁵ is —F, R⁶ is —OH, and R² is

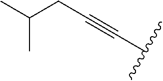 [SMILES: *C#CCC(C)C]

optionally substituted with one, two, or three R^{20b}. In some N embodiments, the compound is a compound of Formula (Ia″-2), R⁴ is cyclopropyl, R⁵ is —F, R⁶ is —OH, and R² is

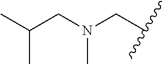 [SMILES: *CN(C)CC(C)C]

optionally substituted with one, two, or three R^{20b}.

In some embodiments, the compound is a compound of Formula (Ia″-2), R⁴ is cyclopropyl, R⁵ is —F, R⁶ is —OH, and R² is

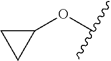 [SMILES: *OC1CC1]

optionally substituted with one, two, or three R^{20k}. In some embodiments, the compound is a compound of Formula (Ia″-2), R⁴ is cyclopropyl, R⁵ is —F, R⁶ is —OH, and R² is

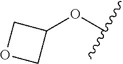 [SMILES: *OC1COC1]

optionally substituted with one, two, or three R^{20k}. In some embodiments, the compound is a compound of Formula (Ia″-2), R⁴ is cyclopropyl, R⁵ is —F, R⁶ is —OH, and R² is,

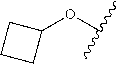 [SMILES: *OC1CCC1]

optionally substituted with one, two, or three R^{20k}. In some embodiments, the compound is a compound of Formula (Ia″-2), R⁴ is cyclopropyl, R⁵ is —F, R⁶ is —OH, and R² is

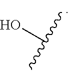 [SMILES: *O]

In some embodiments, the compound is a compound of Formula (Ia″-2), R⁴ is cyclopropyl, R⁵ is —F, R⁶ is —OH, and R² is

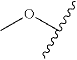 [SMILES: *OC]

optionally substituted with one, two, or three R^{20k}. In some embodiments, the compound is a compound of Formula (Ia″-2), R⁴ is cyclopropyl, R⁵ is —F, R⁶ is —OH, and R² is

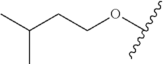 [SMILES: *OCCC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

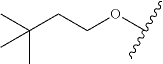 [SMILES: *OCCC(C)(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

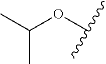 [SMILES: *OC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

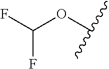 [SMILES: *OC(F)F]

optionally substituted with one R^{20k}. In some embodiments, the compound is a compound of Formula (Ia″-2), R⁴ is cyclopropyl, R⁵ is —F, R⁶ is —OH, and R² is

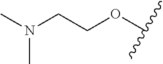 [SMILES: *OCCN(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

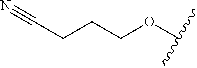 [SMILES: *OCCCC#N]

optionally substituted with one, two, or three R^{20k}. In some embodiments, the compound is a compound of Formula (Ia″-2), R⁴ is cyclopropyl, R⁵ is —F, R⁶ is —OH, and R² is

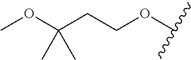 [SMILES: *OCCC(C)(C)OC]

optionally substituted with one, two, or three R^{20k}. In some embodiments, the compound is a compound of Formula (Ia″-2), R⁴ is cyclopropyl, R⁵ is —F, R⁶ is —OH, and R² is

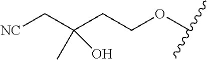 [SMILES: *OCCC(C)(O)CC#N]

optionally substituted with one, two, or three R^{20k}. In some embodiments, the compound is a compound of Formula (Ia″-2), R⁴ is cyclopropyl, R⁵ is —F, R⁶ is —OH, and R² is

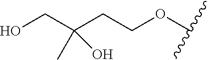 [SMILES: *OCCC(C)(O)CO]

optionally substituted with one, two, or three R^{20k}. In some embodiments, the compound is a compound of Formula (Ia″-2), R⁴ is cyclopropyl, R⁵ is —F, R⁶ is —OH, and R² is

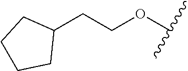 [SMILES: *OCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

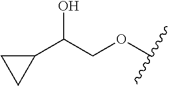 [SMILES: *OCC(O)C1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

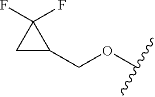 [SMILES: *OCC1CC1(F)F]

optionally substituted with one, two, or three R^{20k}. In some embodiments, the compound is a compound of Formula (Ia″-2), R⁴ is cyclopropyl, R⁵ is —F, R⁶ is —OH, and R² is

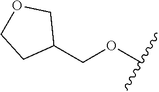 [SMILES: *OCC1CCOC1]

optionally substituted with one, two, or three R^{20k}. In some embodiments, the compound is a compound of Formula (Ia″-2), R⁴ is cyclopropyl, R⁵ is —F, R⁶ is —OH, and R² is

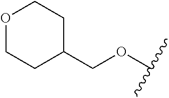 [SMILES: *OCC1CCOCC1]

optionally substituted with one, two, or three R^{20k}. In some embodiments, the compound is a compound of Formula (Ia″-2), R⁴ is cyclopropyl, R⁵ is —F, R⁶ is —OH, and R² is

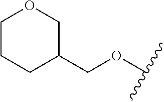 [SMILES: *OCC1CCCOC1]

optionally substituted with one, two, or three R^{20k}In some embodiments, the compound is a compound of Formula (Ia″-2), R⁴ is cyclopropyl, R⁵ is —F, R⁶ is —OH, and R² is

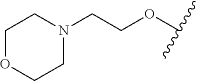 [SMILES: *OCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

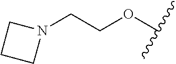 [SMILES: *OCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

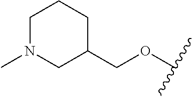 [SMILES: *OCC1CCCN(C)C1]

optionally substituted with one, two, or three R^{20k}. In some embodiments, the compound is a compound of Formula (Ia″-2), R⁴ is cyclopropyl, R⁵ is —F, R⁶ is —OH, and R² is

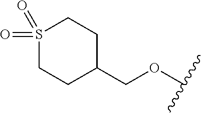 [SMILES: *OCC1CCS(=O)(=O)CC1]

optionally substituted with one, two, or three R^{20k} and optionally substituted with one, two, or three groups independently selected from halogen, oxo, —CN, C₁₋₆ alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

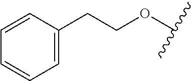 [SMILES: *OCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

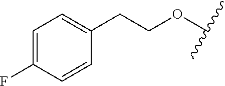 [SMILES: *OCCc1ccc(F)cc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

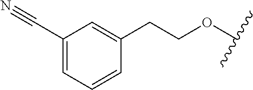 [SMILES: *OCCc1cccc(C#N)c1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is cyclopropyl, R⁵ is —F, R⁶ is —OH, and R² is

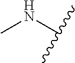 [SMILES: *NC]

optionally substituted with one, two, or three R^{20k}. In some embodiments, the compound is a compound of Formula (Ia″-2), R⁴ is cyclopropyl, R⁵ is —F, R⁶ is —OH, and R² is

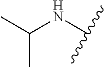 [SMILES: *NC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

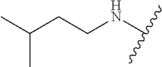 [SMILES: *NCCC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

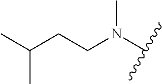 [SMILES: *N(C)CCC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

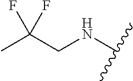 [SMILES: *NCC(C)(F)F]

optionally substituted with one, two, or three R^{20k}. In some embodiments, the compound is a compound of Formula (Ia″-2), R⁴ is cyclopropyl, R⁵ is —F, R⁶ is —OH, and R² is

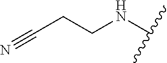 [SMILES: *NCCC#N]

optionally substituted with one, two, or three R^{20k}. In some embodiments, the compound is a compound of Formula (Ia″-2), R⁴ is cyclopropyl, R⁵ is —F, R⁶ is —OH, and R² is

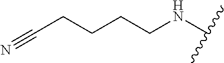 [SMILES: *NCCCCC#N]

optionally substituted with one, two, or three R^{20k}. In some embodiments, the compound is a compound of Formula (Ia″-2), R⁴ is cyclopropyl, R⁵ is —F, R⁶ is —OH, and R² is

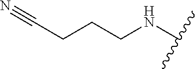 [SMILES: *NCCCC#N]

optionally substituted with one, two, or three R^{20k}. In some embodiments, the compound is a compound of Formula (Ia″-2), R⁴ is cyclopropyl, R⁵ is —F, R⁶ is —OH, and R² is

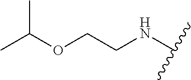 [SMILES: *NCCOC(C)C]

optionally substituted with one, two, or three R^{20k}. In some embodiments, the compound is a compound of Formula (Ia″-2), R⁴ is cyclopropyl, R⁵ is —F, R⁶ is —OH, and R² is

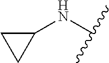 [SMILES: *NC1CC1]

optionally substituted with one, two, or three R^{20k}. In some embodiments, the compound is a compound of Formula (Ia″-2), R⁴ is cyclopropyl, R⁵ is —F, R⁶ is —OH, and R² is

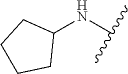 [SMILES: *NC1CCCC1]

optionally substituted with one, two, or three R^{20k}. In some embodiments, the compound is a compound of Formula (Ia″-2), R⁴ is cyclopropyl, R⁵ is —F, R⁶ is —OH, and R² is

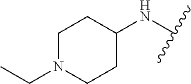 [SMILES: *NC1CCN(CC)CC1]

optionally substituted with one, two, or three R^{20k}. In some embodiments, the compound is a compound of Formula (Ia″-2), R⁴ is cyclopropyl, R⁵ is —F, R⁶ is —OH, and R² is

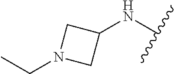 [SMILES: *NC1CN(CC)C1]

optionally substituted with one, two, or three R^{20k}. In some embodiments, the compound is a compound of Formula (Ia″-2), R⁴ is cyclopropyl, R⁵ is —F, R⁶ is —OH, and R² is

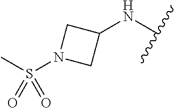 [SMILES: *NC1CN(S(C)(=O)=O)C1]

optionally substituted with one, two, or three R^{20k}. In some embodiments, the compound is a compound of Formula (Ia″-2), R⁴ is cyclopropyl, R⁵ is —F, R⁶ is —OH, and R² is

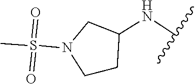 [SMILES: *NC1CCN(S(C)(=O)=O)C1]

optionally substituted with one, two, or three R^{20k}. In some embodiments, the compound is a compound of Formula (Ia″-2), R⁴ is cyclopropyl, R⁵ is —F, R⁶ is —OH, and R² is

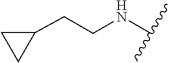 [SMILES: *NCCC1CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

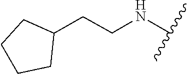 [SMILES: *NCCC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

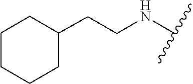 [SMILES: *NCCC1CCCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

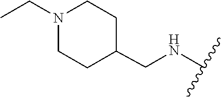 [SMILES: *NCC1CCN(CC)CC1]

optionally substituted with one, two, or three R^{20k}. In some embodiments, the compound is a compound of Formula (Ia″-2), R⁴ is cyclopropyl, R⁵ is —F, R⁶ is —OH, and R² is

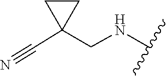 [SMILES: *NCC1(C#N)CC1]

optionally substituted with one, two, or three R^{20k}. In some embodiments, the compound is a compound of Formula (Ia″-2), R⁴ is cyclopropyl, R⁵ is —F, R⁶ is —OH, and R² is

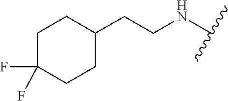 [SMILES: *NCCC1CCC(F)(F)CC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

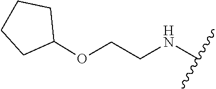 [SMILES: *NCCOC1CCCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

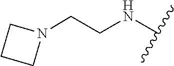 [SMILES: *NCCN1CCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

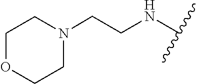 [SMILES: *NCCN1CCOCC1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

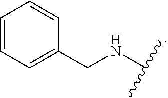 [SMILES: *NCc1ccccc1]

In some embodiments, the compound is a compound of Formula (Ia″-2), R⁴ is cyclopropyl, R⁵ is —F, R⁶ is —OH, and R² is

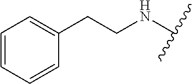 [SMILES: *NCCc1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

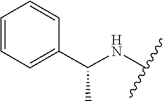 [SMILES: *N[C@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

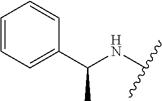 [SMILES: *N[C@@H](C)c1ccccc1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

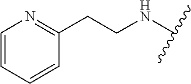 [SMILES: *NCCc1ccccn1]

optionally substituted with one, two, or three R^{20k} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is cyclopropyl, R⁵ is —F, R⁶ is —OH, and R² is

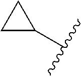 [SMILES: *C1CC1]

optionally substituted with one, two, or three R^{20b}. In some embodiments, the compound is a compound of Formula (Ia″-2), R⁴ is cyclopropyl, R⁵ is —F, R⁶ is —OH, and R² is

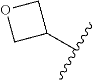 [SMILES: *C1COC1]

optionally substituted with one, two, or three R^{20b}. In some embodiments, the compound is a compound of Formula (Ia″-2), R⁴ is cyclopropyl, R⁵ is —F, R⁶ is —OH, and R² is

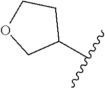 [SMILES: *C1CCOC1]

optionally substituted with one, two, or three R^{20b}. In some embodiments, the compound is a compound of Formula (Ia″-2), R⁴ is cyclopropyl, R⁵ is —F, R⁶ is —OH, and R² is

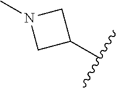 [SMILES: *C1CN(C)C1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

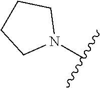 [SMILES: *N1CCCC1]

optionally substituted with one, two, or three R^{20b}. In some embodiments, the compound is a compound of Formula (Ia″-2), R⁴ is cyclopropyl, R⁵ is —F, R⁶ is —OH, and R² is

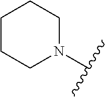 [SMILES: *N1CCCCC1]

optionally substituted with one, two, or three R^{20b}. In some embodiments, the compound is a compound of Formula (Ia″-2), R⁴ is cyclopropyl, R⁵ is —F, R⁶ is —OH, and R² is

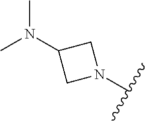 [SMILES: *N1CC(N(C)C)C1]

optionally substituted with one, two, or three R^{20b}. In some embodiments, the compound is a compound of Formula (Ia″-2), R⁴ is cyclopropyl, R⁵ is —F, R⁶ is —OH, and R² is

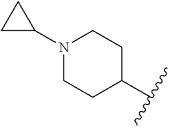 [SMILES: *C1CCN(C2CC2)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

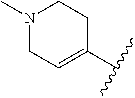 [SMILES: *C1=CCN(C)CC1]

optionally substituted with one, two, or three R^{20b} and optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R² is

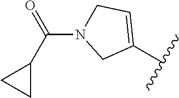 [SMILES: *C1=CCN(C(=O)C2CC2)C1]

optionally substituted with one, two, or three R^{20b}. In some embodiments, the compound is a compound of Formula (Ia″-2), R⁴ is cyclopropyl, R⁵ is —F, R⁶ is —OH, and R² is

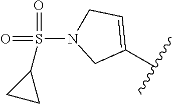 [SMILES: *C1=CCN(S(=O)(=O)C2CC2)C1]

optionally substituted with one, two, or three R^{20b}. In some embodiments, the compound is a compound of Formula (Ia″-2), R⁴ is cyclopropyl, R⁵ is —F, R⁶ is —OH, and R² is

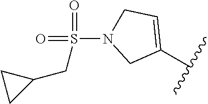 [SMILES: *C1=CCN(S(=O)(=O)CC2CC2)C1]

optionally substituted with one, two, or three R^{20b}. In some embodiments, the compound is a compound of Formula (Ia″-2), R⁴ is cyclopropyl, R⁵ is —F, R⁶ is —OH, and R² is

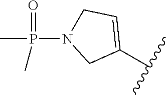 [SMILES: *C1=CCN(P(C)(C)=O)C1]

optionally substituted with one, two, or three R^{20b}In some embodiments, the compound is a compound of Formula (Ia″-2), R⁴ is cyclopropyl, R⁵ is —F, R⁶ is —OH, and R² is

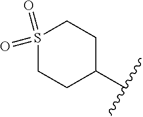 [SMILES: *C1CCS(=O)(=O)CC1]

optionally substituted with one, two, or three R^{20b}. In some embodiments, the compound is a compound of Formula (Ia″-2), R⁴ is cyclopropyl, R⁵ is —F, R⁶ is —OH, and R² is

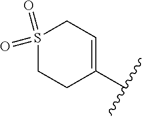 [SMILES: *C1=CCS(=O)(=O)CC1]

optionally substituted with one, two, or three R^{20b}. In some embodiments, the compound is a compound of Formula (Ia″-2), R⁴ is cyclopropyl, R⁵ is —F, R⁶ is —OH, and R² is

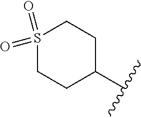 [SMILES: *C1CCS(=O)(=O)CC1]

optionally substituted with one, two, or three R^{20b}. In some embodiments, the compound is a compound of Formula (Ia″-2), R⁴ is cyclopropyl, R⁵ is —F, R⁶ is —OH, and R² is

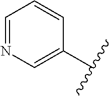 [SMILES: *c1cccnc1]

optionally substituted with one, two, or three R^{20b}. In some embodiments, the compound is a compound of Formula (Ia″-2), R⁴ is cyclopropyl, R⁵ is —F, R⁶ is —OH, and R² is

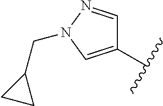 [SMILES: *c1cnn(CC2CC2)c1]

optionally substituted with one, two, or three R^{20b} and optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is cyclopropyl, R⁵ is —F, R⁶ is —OH, and R² is independently hydrogen. In some embodiments, the compound is a compound of Formula (Ia″-2), R⁴ is cyclopropyl, R⁵ is —F, R⁶ is —OH, and R² is independently halogen. In some embodiments, the compound is a compound of Formula (Ia″-2), R⁴ is cyclopropyl, R⁵ is —F, R⁶ is —OH, and R² is independently —CN. In some embodiments, the compound is a compound of Formula (Ia″-2), R⁴ is cyclopropyl, R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl. In some embodiments, the compound is a compound of Formula (Ia″-2), R⁴ is cyclopropyl, R⁵ is —F, R⁶ is —OH, and R² is independently —OR¹². In some embodiments, the compound is a compound of Formula (Ia″-2), R⁴ is cyclopropyl, R⁵ is —F, R⁶ is —OH, and R² is independently —SR¹². In some embodiments, the compound is a compound of Formula (Ia″-2), R⁴ is cyclopropyl, R⁵ is —F, R⁶ is —OH, and R² is independently —N(R¹²)(R¹³). In some embodiments, the compound is a compound of Formula (Ia″-2), R⁴ is cyclopropyl, R⁵ is —F, R⁶ is —OH, and R² is independently —C(O)OR¹². In some embodiments, the compound is a compound of Formula (Ia″-2), R⁴ is cyclopropyl, R⁵ is —F, R⁶ is —OH, and R² is independently —OC(O)N(R¹²)(R¹³). In some embodiments, the compound is a compound of Formula (Ia″-2), R⁴ is cyclopropyl, R⁵ is —F, R⁶ is —OH, and R² is independently —N(R¹⁴)C(O)N(R¹²)(R¹³). In some embodiments, the compound is a compound of Formula (Ia″-2), R⁴ is cyclopropyl, R⁵ is —F, R⁶ is —OH, and R² is independently —N(R¹⁴)C(O)OR¹². In some embodiments, the compound is a compound of Formula (Ia″-2), R⁴ is cyclopropyl, R⁵ is —F, R⁶ is —OH, and R² is independently —N(R¹⁴)S(O)₂R¹⁵. In some embodiments, the compound is a compound of Formula (Ia″-2), R⁴ is cyclopropyl, R⁵ is —F, R⁶ is —OH, and R² is independently —C(O)R¹⁵. In some embodiments, the compound is a compound of Formula (Ia″-2), R⁴ is cyclopropyl, R⁵ is —F, R⁶ is —OH, and R² is independently —S(O)R¹⁵. In some embodiments, the compound is a compound of Formula (Ia″-2), R⁴ is cyclopropyl, R⁵ is —F, R⁶ is —OH, and R² is independently —OC(O)R¹⁵. In some embodiments, the compound is a compound of Formula (Ia″-2), R⁴ is cyclopropyl, R⁵ is —F, R⁶ is —OH, and R² is independently —C(O)N(R¹²)(R¹³). In some embodiments, the compound is a compound of Formula (Ia″-2), R⁴ is cyclopropyl, R⁵ is —F, R⁶ is —OH, and R² is independently —C(O)C(O)N(R¹²)(R¹³). In some embodiments, the compound is a compound of Formula (Ia″-2), R⁴ is cyclopropyl, R⁵ is —F, R⁶ is —OH, and R² is independently —N(R¹⁴)C(O)R¹⁵. In some embodiments, the compound is a compound of Formula (Ia″-2), R⁴ is cyclopropyl, R⁵ is —F, R⁶ is —OH, and R² is independently —S(O)₂R¹⁵. In some embodiments, the compound is a compound of Formula (Ia″-2), R⁴ is cyclopropyl, R⁵ is —F, R⁶ is —OH, and R² is independently —S(O)₂N(R¹²)(R¹³). In some embodiments, the compound is a compound of Formula (Ia″-2), R⁴ is cyclopropyl, R⁵ is —F, R⁶ is —OH, and R² is independently —S(═O)(═NH)N(R¹²)(R¹³). In some embodiments, the compound is a compound of Formula (Ia″-2), R⁴ is cyclopropyl, R⁵ is —F, R⁶ is —OH, and R² is independently —CH₂C(O)N(R¹²)(R¹³). In some embodiments, the compound is a compound of Formula (Ia″-2), R⁴ is cyclopropyl, R⁵ is —F, R⁶ is —OH, and R² is independently —CH₂N(R¹⁴)C(O)R¹⁵. In some embodiments, the compound is a compound of Formula (Ia″-2), R⁴ is cyclopropyl, R⁵ is —F, R⁶ is —OH, and R² is independently —CH₂S(O)₂R¹⁵. In some embodiments, the compound is a compound of Formula (Ia″-2), R⁴ is cyclopropyl, R⁵ is —F, R⁶ is —OH, and R² is independently —CH₂S(O)₂N(R¹²)(R¹³). In some embodiments, the compound is a compound of Formula (Ia″-2), R⁴ is cyclopropyl, R⁵ is —F, R⁶ is —OH, and R² is independently C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl substituted with one, two, or three R^{20b}. In some embodiments, the compound is a compound of Formula (Ia″-2), R⁴ is cyclopropyl, R⁵ is —F, R⁶ is —OH, and R² is independently C₁₋₆alkyl substituted with one, two, or three R^{20b}. In some embodiments, the compound is a compound of Formula (Ia″-2), R⁴ is cyclopropyl, R⁵ is —F, R⁶ is —OH, and R² is independently C₂₋₆alkenyl substituted with one, two, or three R^{20b}In some embodiments, the compound is a compound of Formula (Ia″-2), R⁴ is cyclopropyl, R⁵ is —F, R⁶ is —OH, and R² is independently C₂₋₆alkynyl substituted with one, two, or three R^{20b}. In some embodiments, the compound is a compound of Formula (Ia″-2), R⁴ is cyclopropyl, R⁵ is —F, R⁶ is —OH, and R² is independently C₃₋₆cycloalkyl substituted with one, two, or three R^{20b}. In some embodiments, the compound is a compound of Formula (Ia″-2), R⁴ is cyclopropyl, R⁵ is —F, R⁶ is —OH, and R² is independently C₂₋₉heterocycloalkyl substituted with one, two, or three R^{20b}. In some embodiments, the compound is a compound of Formula (Ia″-2), R⁴ is cyclopropyl, R⁵ is —F, R⁶ is —OH, and R² is independently C₆₋₁₀aryl substituted with one, two, or three R^{20b}. In some embodiments, the compound is a compound of Formula (Ia″-2), R⁴ is cyclopropyl, R⁵ is —F, R⁶ is —OH, and R² is independently C₁₋₉ heteroaryl substituted with one, two, or three R^{20b}. In some embodiments, the compound is a compound of Formula (Ia″-2), R⁴ is cyclopropyl, R⁵ is —F, R⁶ is —OH, and R² is independently C₁₋₆alkyl. In some embodiments, the compound is a compound of Formula (Ia″-2), R⁴ is cyclopropyl, R⁵ is —F, R⁶ is —OH, and R² is independently C₂₋₆alkenyl. In some embodiments, the compound is a compound of Formula (Ia″-2), R⁴ is cyclopropyl, R⁵ is —F, R⁶ is —OH, and R² is independently C₂₋₆alkynyl. In some embodiments, the compound is a compound of Formula (Ia″-2), R⁴ is cyclopropyl, R⁵ is —F, R⁶ is —OH, and R² is independently C₃₋₆cycloalkyl. In some embodiments, the compound is a compound of Formula (Ia″-2), R⁴ is cyclopropyl, R⁵ is —F, R⁶ is —OH, and R² is independently C₂₋₉heterocycloalkyl. In some embodiments, the compound is a compound of Formula (Ia″-2), R⁴ is cyclopropyl, R⁵ is —F, R⁶ is —OH, and R² is independently C₆₋₁₀aryl. In some embodiments, the compound is a compound of Formula (Ia″-2), R⁴ is cyclopropyl, R⁵ is —F, R⁶ is —OH, and R² is independently C₁₋₉heteroaryl. In some embodiments, the compound is a compound of Formula (Ia″-2), R⁴ is cyclopropyl, R⁵ is —F, R⁶ is —OH, and R² is independently C₁alkyl. In some embodiments, the compound is a compound of Formula (Ia″-2), R⁴ is cyclopropyl, R⁵ is —F, R⁶ is —OH, and R² is independently C₂alkyl. In some embodiments, the compound is a compound of Formula (Ia″-2), R⁴ is cyclopropyl, R⁵ is —F, R⁶ is —OH, and R² is independently C₃alkyl. In some embodiments, the compound is a compound of Formula (Ia″-2), R⁴ is cyclopropyl, R⁵ is —F, R⁶ is —OH, and R² is independently C₄alkyl. In some embodiments, the compound is a compound of Formula (Ia″-2), R⁴ is cyclopropyl, R⁵ is —F, R⁶ is —OH, and R² is independently C₅alkyl. In some embodiments, the compound is a compound of Formula (Ia″-2), R⁴ is cyclopropyl, R⁵ is —F, R⁶ is —OH, and R² is independently C₆alkyl.

In some embodiments, the compound is a compound of Formula (Ia″-2), R⁴ is cyclopropyl, R⁵ is —F, R⁶ is —OH, and R² is independently —OH. In some embodiments, the compound is a compound of Formula (Ia″-2), R⁴ is cyclopropyl, R⁵ is —F, R⁶ is —OH, and R² is independently —SH. In some embodiments, the compound is a compound of Formula (Ia″-2), R⁴ is cyclopropyl, R⁵ is —F, R⁶ is —OH, and R² is independently —NH₂. In some embodiments, the compound is a compound of Formula (Ia″-2), R⁴ is cyclopropyl, R⁵ is —F, R⁶ is —OH, and R² is independently —C(O)OH. In some embodiments, the compound is a compound of Formula (Ia″-2), R⁴ is cyclopropyl, R⁵ is —F, R⁶ is —OH, and R² is independently —OC(O)NH₂. In some embodiments, the compound is a compound of Formula (Ia″-2), R⁴ is cyclopropyl, R⁵ is —F, R⁶ is —OH, and R² is independently —NHC(O)NH₂. In some embodiments, the compound is a compound of Formula (Ia″-2), R⁴ is cyclopropyl, R⁵ is —F, R⁶ is —OH, and R² is independently —NHC(O)OH. In some embodiments, the compound is a compound of Formula (Ia″-2), R⁴ is cyclopropyl, R⁵ is —F, R⁶ is —OH, and R² is independently —NHS(O)₂R¹⁵. In some embodiments, the compound is a compound of Formula (Ia″-2), R⁴ is cyclopropyl, R⁵ is —F, R⁶ is —OH, and R² is independently —C(O)R¹⁵. In some embodiments, the compound is a compound of Formula (Ia″-2), R⁴ is cyclopropyl, R⁵ is —F, R⁶ is —OH, and R² is independently —S(O)R¹⁵. In some embodiments, the compound is a compound of Formula (Ia″-2), R⁴ is cyclopropyl, R⁵ is —F, R⁶ is —OH, and R² is independently —OC(O)R¹⁵. In some embodiments, the compound is a compound of Formula (Ia″-2), R⁴ is cyclopropyl, R⁵ is —F, R⁶ is —OH, and R² is independently —OC(O)CH₃. In some embodiments, the compound is a compound of Formula (Ia″-2), R⁴ is cyclopropyl, R⁵ is —F, R⁶ is —OH, and R² is independently —C(O)NH₂. In some embodiments, the compound is a compound of Formula (Ia″-2), R⁴ is cyclopropyl, R⁵ is —F, R⁶ is —OH, and R² is independently —C(O)C(O)NH₂. In some embodiments, the compound is a compound of Formula (Ia″-2), R⁴ is cyclopropyl, R⁵ is —F, R⁶ is —OH, and R² is independently —NHC(O)R¹⁵. In some embodiments, the compound is a compound of Formula (Ia″-2), R⁴ is cyclopropyl, R⁵ is —F, R⁶ is —OH, and R² is independently —S(O)₂R¹⁵. In some embodiments, the compound is a compound of Formula (Ia″-2), R⁴ is cyclopropyl, R⁵ is —F, R⁶ is —OH, and R² is independently —S(O)₂N(R¹²)(R¹³). In some embodiments, the compound is a compound of Formula (Ia″-2), R⁴ is cyclopropyl, R⁵ is —F, R⁶ is —OH, and R² is independently —S(═O)(═NH)NH₂. In some embodiments, the compound is a compound of Formula (Ia″-2), R⁴ is cyclopropyl, R⁵ is —F, R⁶ is —OH, and R² is independently —CH₂C(O)NH₂. In some embodiments, the compound is a compound of Formula (Ia″-2), R⁴ is cyclopropyl, R⁵ is —F, R⁶ is —OH, and R² is independently —CH₂NHC(O)R¹⁵. In some embodiments, the compound is a compound of Formula (Ia″-2), R⁴ is cyclopropyl, R⁵ is —F, R⁶ is —OH, and R² is independently —CH₂S(O)₂R¹⁵. In some embodiments, the compound is a compound of Formula (Ia″-2), R⁴ is cyclopropyl, R⁵ is —F, R⁶ is —OH, and R² is independently —CH₂S(O)₂NH₂.

In some embodiments, the compound is a compound of Formula (Ia″-2), R⁴ is cyclopropyl, R⁵ is —F, R⁶ is —OH, and R² is independently —NH(R¹²). In some embodiments, the compound is a compound of Formula (Ia″-2), R⁴ is cyclopropyl, R⁵ is —F, R⁶ is —OH, and R² is independently —OC(O)N(R¹²)(R¹³). In some embodiments, the compound is a compound of Formula (Ia″-2), R⁴ is cyclopropyl, R⁵ is —F, R⁶ is —OH, and R² is independently —NHC(O)N(R¹²). In some embodiments, the compound is a compound of Formula (Ia″-2), R⁴ is cyclopropyl, R⁵ is —F, R⁶ is —OH, and R² is independently —NHC(O)OR¹². In some embodiments, the compound is a compound of Formula (Ia″-2), R⁴ is cyclopropyl, R⁵ is —F, R⁶ is —OH, and R² is independently —C(O)N(R¹²)(R¹³). In some embodiments, the compound is a compound of Formula (Ia″-2), R⁴ is cyclopropyl, R⁵ is —F, R⁶ is —OH, and R² is independently —C(O)C(O)NH(R¹²). In some embodiments, the compound is a compound of Formula (Ia″-2), R⁴ is cyclopropyl, R⁵ is —F, R⁶ is —OH, and R² is independently —S(O)₂NH(R¹²). In some embodiments, the compound is a compound of Formula (Ia″-2), R⁴ is cyclopropyl, R⁵ is —F, R⁶ is —OH, and R² is independently —S(═O)(═NH)NH(R¹²). In some embodiments, the compound is a compound of Formula (Ia″-2), R⁴ is cyclopropyl, R⁵ is —F, R⁶ is —OH, and R² is independently —CH₂C(O)NH(R¹²). In some embodiments, the compound is a compound of Formula (Ia″-2), R⁴ is cyclopropyl, R⁵ is —F, R⁶ is —OH, and R² is independently —CH₂S(O)₂NH(R¹²).

In some embodiments, the compound is a compound of Formula (Ia″-2), R⁴ is cyclopropyl, R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In some embodiments, the compound is a compound of Formula (Ia″-2), R⁴ is cyclopropyl, R⁵ is —F, R⁶ is —OH, and R² is hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}.

In some embodiments, the compound is a compound of Formula (Ia″-2), R⁴ is cyclopropyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

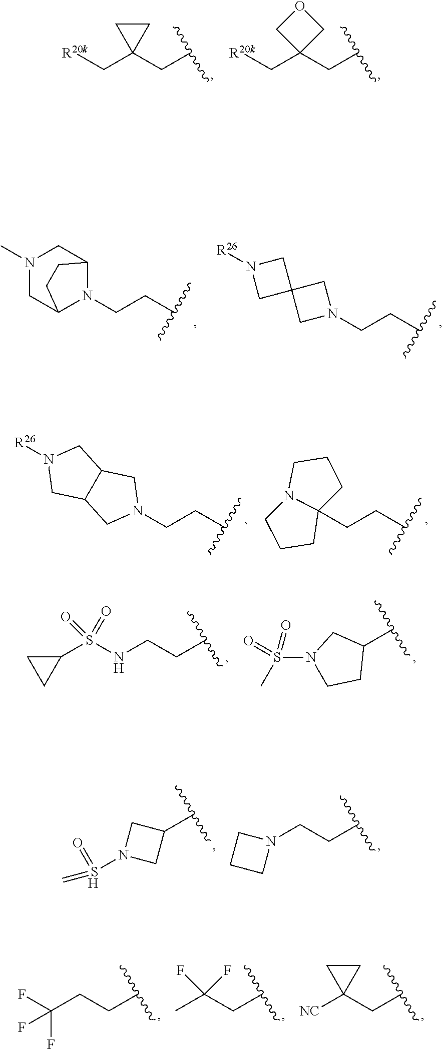

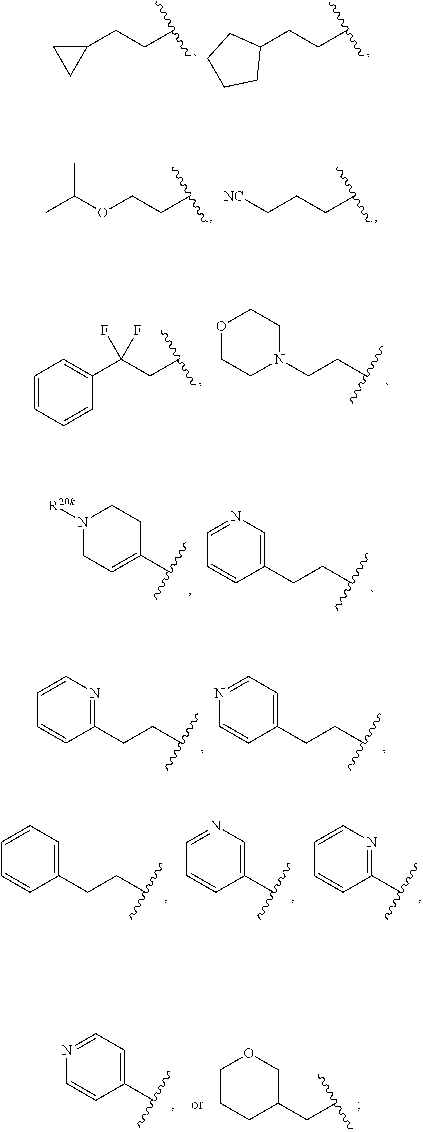

R¹⁵ is independently

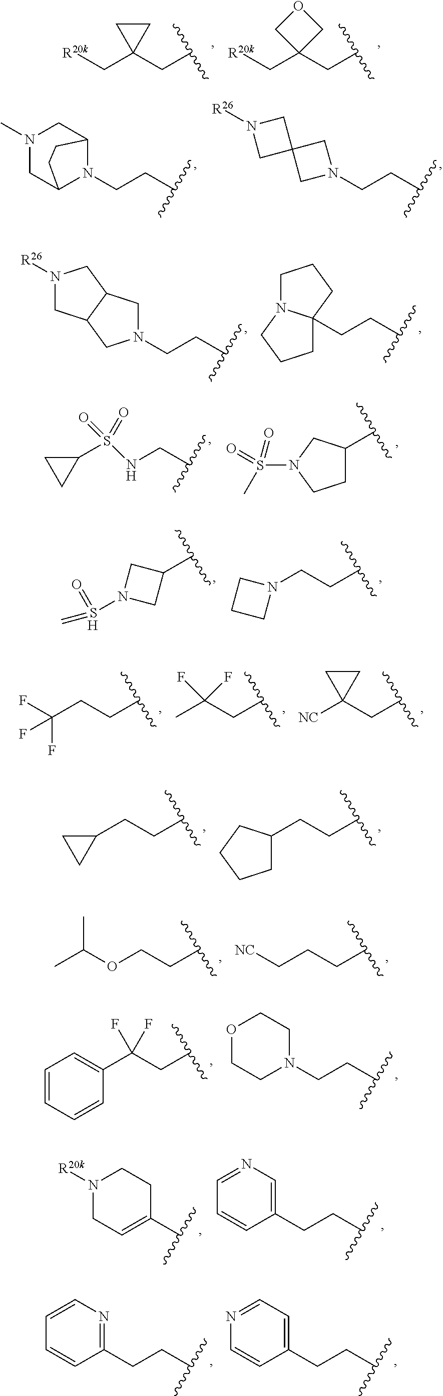

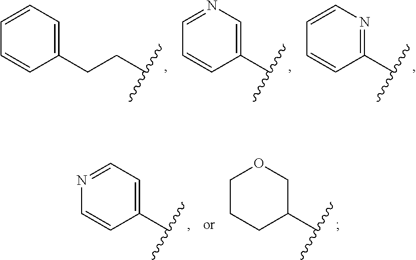

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R²⁶ is independently selected from —CN, C₁₋₆alkyl, C₁₋₆ haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is cyclopropyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² is independently

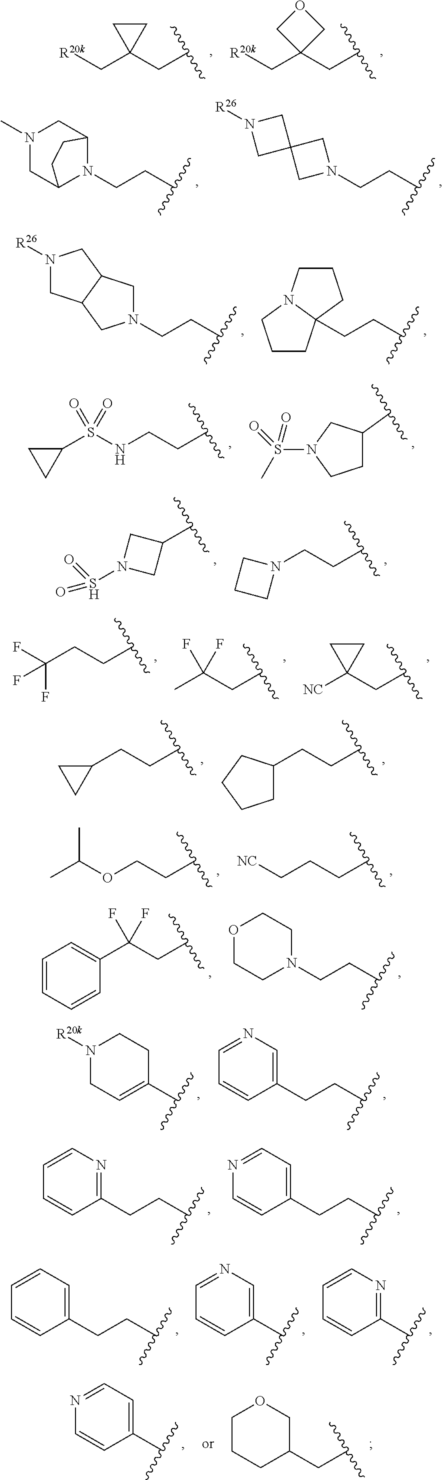

R¹⁵ is independently

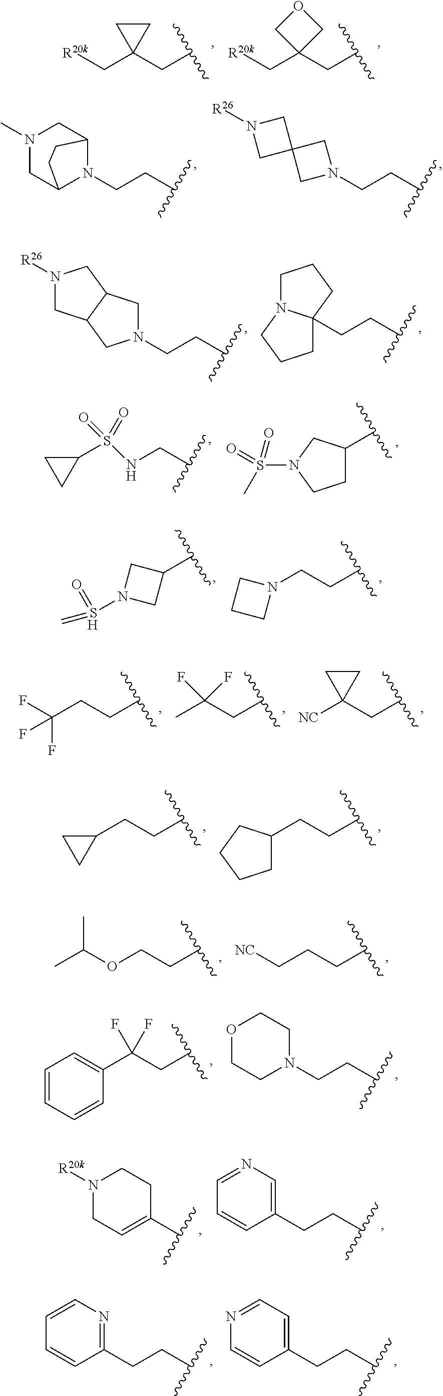

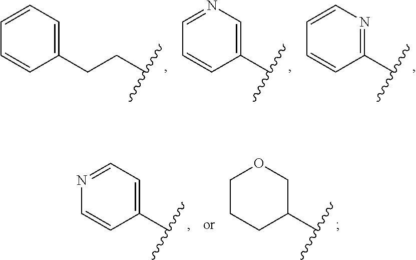

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R²⁶ is independently selected from —CN, C₁₋₆alkyl, C₁₋₆ haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is cyclopropyl, R⁵ is —F, R⁶ is —OH, and R² is selected from

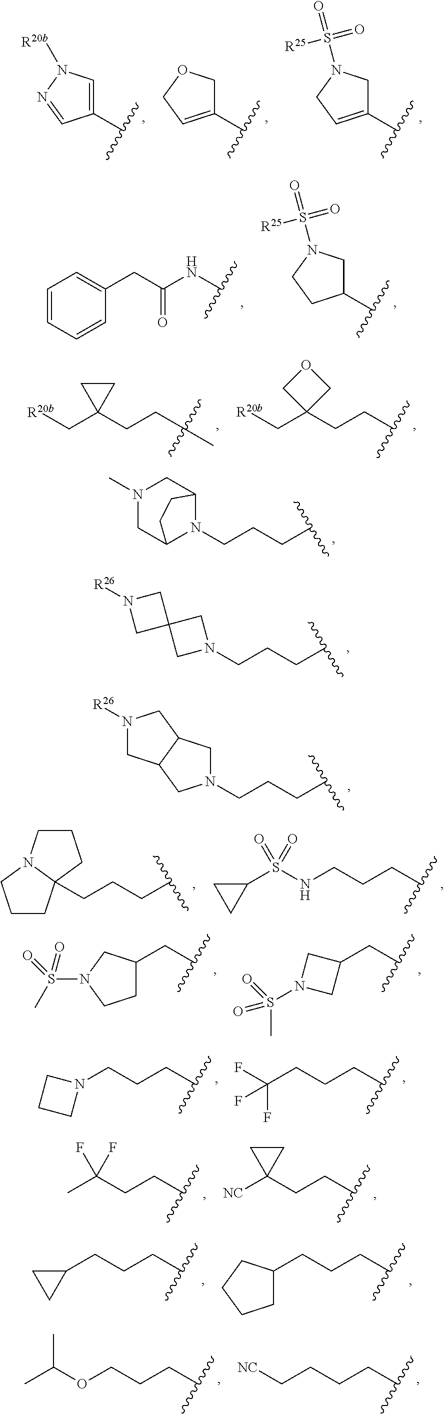

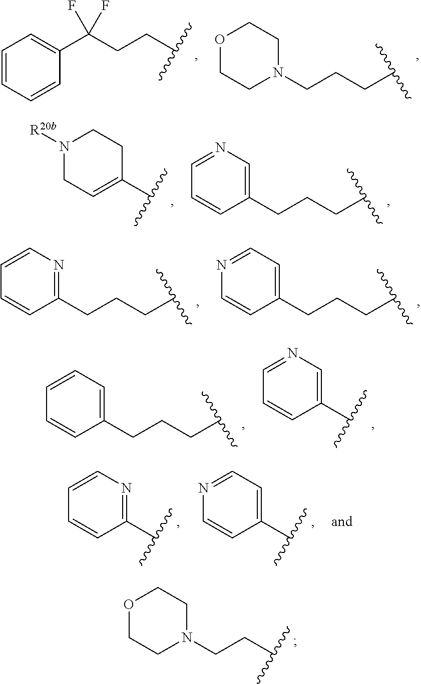

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments, the compound is a compound of Formula (Ia″-2), R⁴ is cyclopropyl, R⁵ is —F, R⁶ is —OH, and R²⁶ is independently selected from —CN, C₁₋₆alkyl, C₁₋₆haloalkyl, C₁₋₆alkoxy, C₁₋₆haloalkoxy, —C(O)OR²², —C(O)N(R²²)(R²³), —C(O)C(O)N(R²²)(R²³), and —C(O)R²⁵.

In some embodiments, the compound is a compound of Formula (Ia″-2), R⁴ is cyclopropyl, R⁵ is —F, R⁶ is —OH, and R² is selected from —OR¹², —N(R¹²)(R¹³), —C(O)R¹⁵, and —C(O)N(R¹²)(R¹³); R¹³ is hydrogen;

R¹² and R¹⁵ are independently

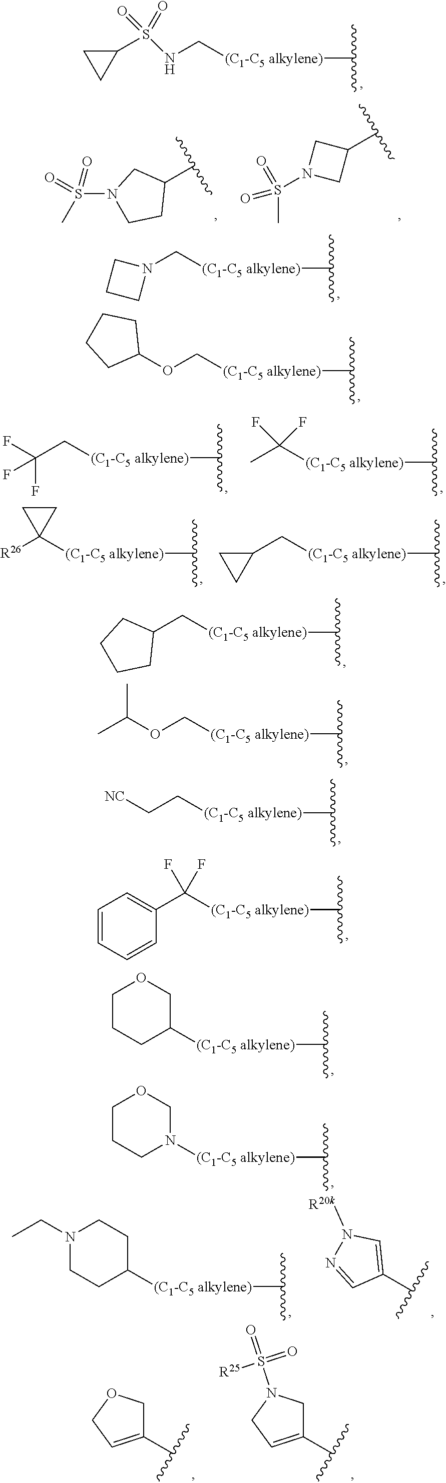

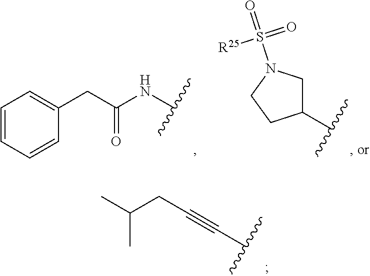

and R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, the compound is a compound of Formula (Ia″-2), R⁴ is cyclopropyl, R⁵ is —F, R⁶ is —OH, and R² is independently

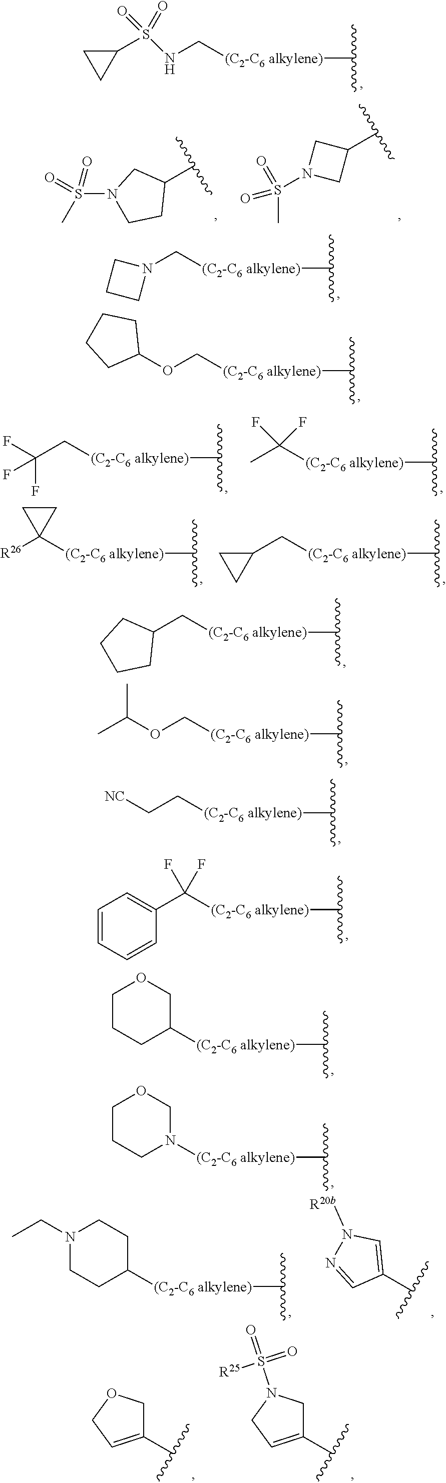

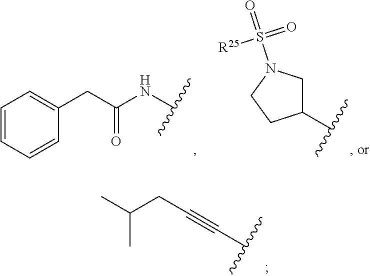

and R²⁶ is independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In an aspect is provided a compound of Formula (I-3), or a pharmaceutically acceptable salt or solvate thereof:

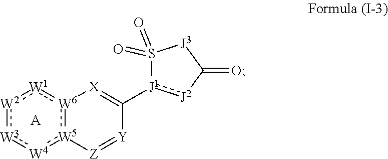

wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R^{2a}), C(O), S(O), S(O)₂, S(O)(NR²), S(O)(R²), or P(O)(R²);
W³ is N, N(R³), C(R³), C(R³)(R^{3a}), C(O), S(O), S(O)₂, S(O)(NR³), S(O)(R³), or P(O)(R³);
W⁴ is N, N(R⁴), C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein at least one of W¹, W², W³, W⁴, W⁵, and W⁶ is N, N(R¹), N(R²), N(R³), or N(R⁴); and
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰);
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR⁶b) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl; [symbol]
[symbol] indicates a single or double bond such that all valences are satisfied; and provided that:
at least two of Ring A [symbol] are double bonds.

In some embodiments, X is C(R⁵).

In some embodiments, Y is C(R⁶).

In some embodiments, Z is C(R⁷).

In some embodiments, J¹ is N and J² is C(R⁹)(R^{9a}).

In some embodiments, J¹ is C(R⁸) and J² is C(R⁹)(R^{9a}).

In some embodiments, J² is CH₂.

In some embodiments, J¹ is C and J² is C(R⁹).

The compound of claim 8, or a pharmaceutically acceptable salt or solvate thereof, wherein J² is C(H).

In some embodiments, J³ is N(H).

In some embodiments, the subject compound, or a pharmaceutically acceptable salt or solvate thereof, has the structure of Formula (Ia-3), (Ib-3), or (Ic-3):

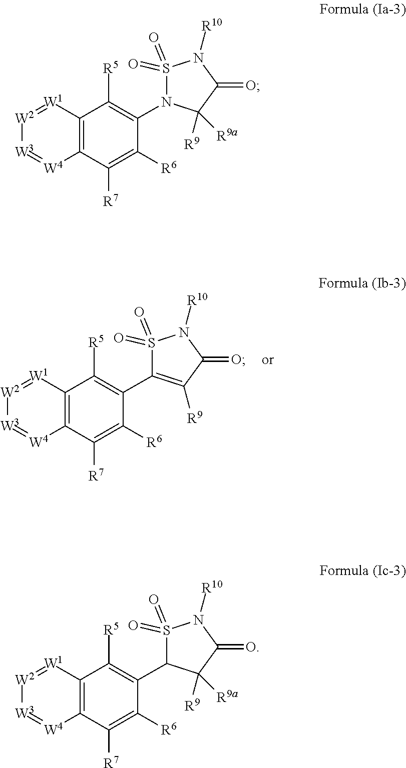

In some embodiments, the subject compound, or a pharmaceutically acceptable salt or solvate thereof, has the structure of Formula (Ia-3):

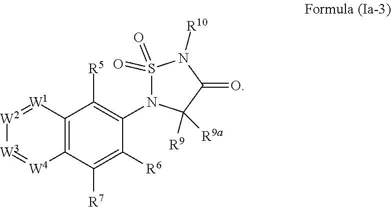

In some embodiments, W¹ is N, W² is C(R²), W³ is C(R³), and W⁴ is C(R⁴).

In some embodiments, R² is hydrogen, R³ is hydrogen, and R⁴ is hydrogen.

In some embodiments, the subject compound, or a pharmaceutically acceptable salt or solvate thereof, has the structure of Formula (Ig-3), (Ih-3), or (Ii-3):

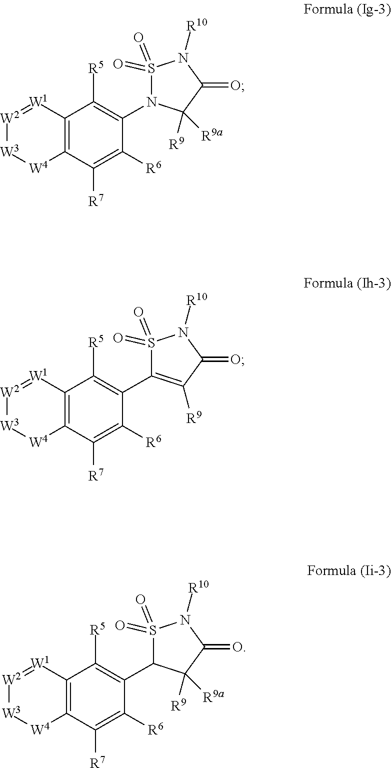

In some embodiments, the subject compound, or a pharmaceutically acceptable salt or solvate thereof, has the structure of Formula (Ig-3):

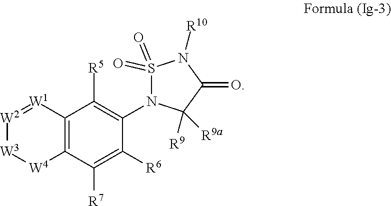

In some embodiments, W¹ is C(R¹) and W² is C(R²).

In some embodiments, R¹ is hydrogen and R² is hydrogen.

In some embodiments, W³ is C(O) and W⁴ is N(R⁴).

In some embodiments, R⁴ is methyl.

In an aspect is provided a acompound of Formula (III-3), or a pharmaceutically acceptable salt or solvate thereof:

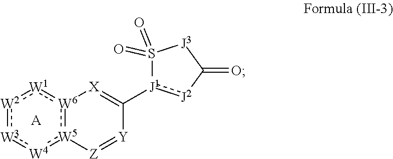

wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R²), C(O), S(O), S(O)₂, S(O)(NR²), S(O)(R²), or P(O)(R²);
W³ is N, N(R³), C(R³), C(R³)(R^{3a}), C(O), S(O), S(O)₂, S(O)(NR³), S(O)(R³), or P(O)(R³);
W⁴ is N, N(R⁴), C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein
at least one of W¹, W², W³, W⁴, W⁵, and W⁶ is N, N(R¹), N(R²), N(R³), N(R⁴), or O; and at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_9 S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁_.heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁_.heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied.

In an aspect is provided a compound of Formula (II-3), or a pharmaceutically acceptable salt or solvate thereof:

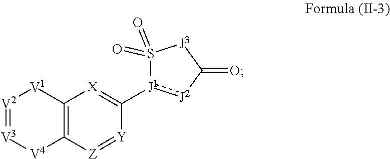

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_9 S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl; and
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl.

In some embodiments, R⁵ is selected from hydrogen and halogen.

In some embodiments, R⁶ is —OH.

In some embodiments, R⁷ is hydrogen.

In some embodiments, R⁹ is hydrogen, R^{9a} is hydrogen, and R¹⁰ is hydrogen.

In some embodiments, R² is —OR¹²; and R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}.

In some embodiments, R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

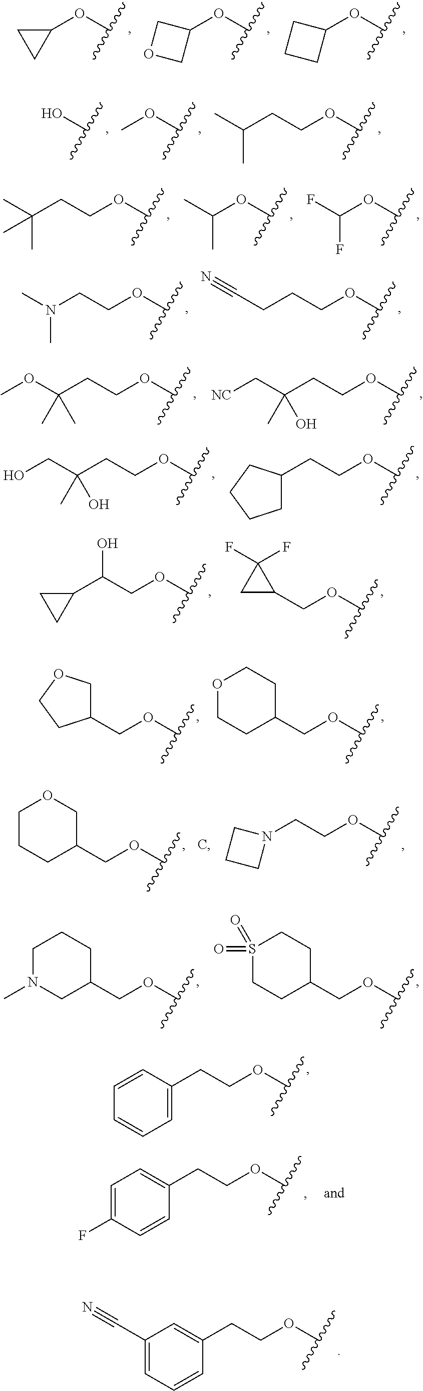

In some embodiments, R² is —N(R¹²)(R¹³); R¹³ is independently hydrogen; and R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three R^{20k}.

In some embodiments, R^{20k} is independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

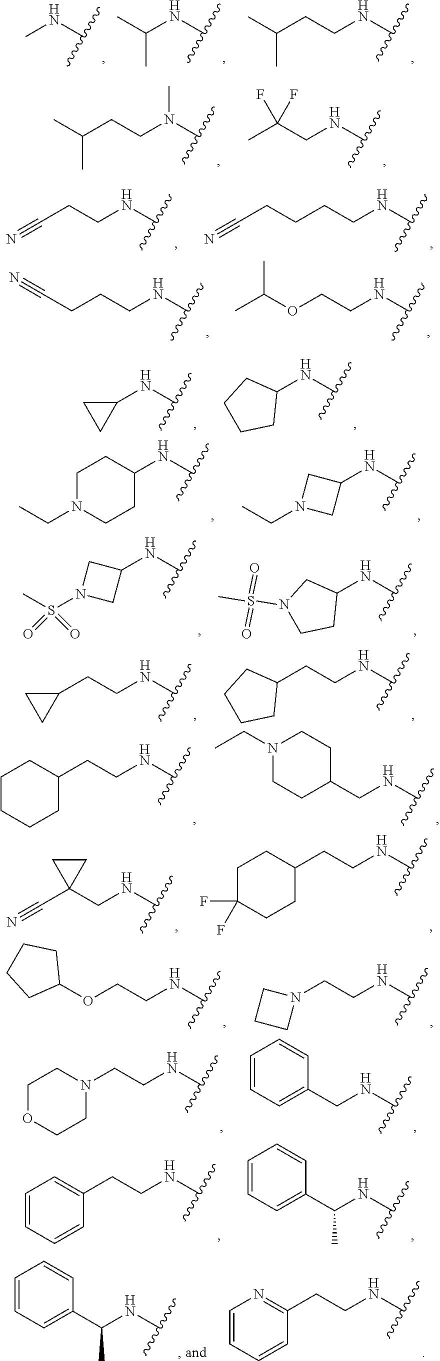

In some embodiments, R² is selected from C₁₋₆alkyl, C₂₋₆alkynyl, C₃₋₆cycloalkcyl, C₂₋₉heterocycloalkyl, C₆₋₁₀aryl, and C₁₋₉ heteroaryl, wherein C₁₋₆alkyl, C₂₋₆alkynyl, C₃₋₆cycloalkcyl, C₂₋₉heterocycloalkcyl, C₆₋₁₀aryl, and C₁₋₉heteroaryl are optionally substituted with one, two, or three R^{20b}.

In some embodiments, R^{20b} is independently selected from oxo, —CN, halogen, C₁₋₆alkyl, C₃₋₆cycloalkyl, —N(R²²)(R²³), —C(O)R²⁵, —S(O)₂R²⁵, and —P(O)(R²⁵)₂, wherein C₁₋₆alkyl and C₃₋₆cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³)q C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from is,

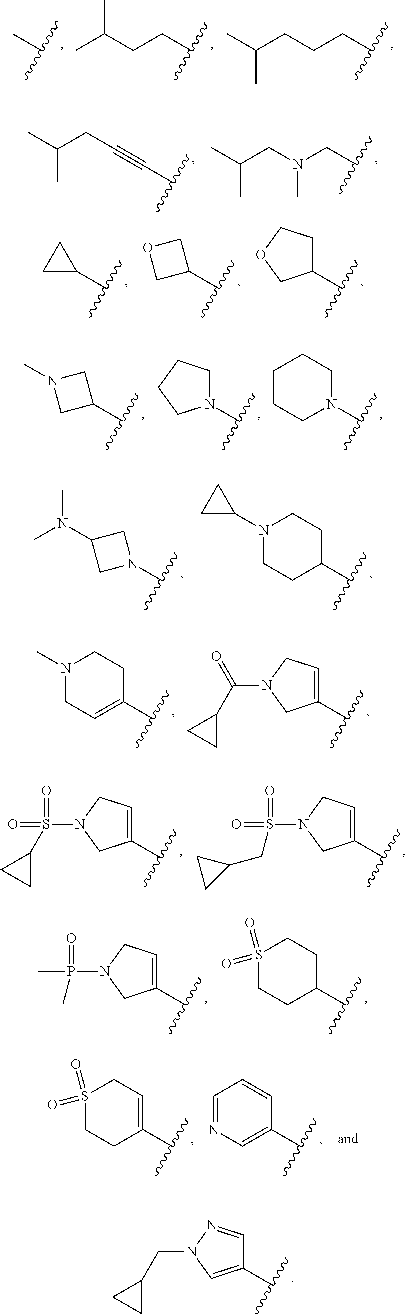

In some embodiments, R² is selected from C₁₋₆alkyl, C₂₋₉heterocycloalkyl, C₁₋₉heteroaryl, —OR¹², —N(R¹²)(R¹³), —C(O)R¹⁵, —C(O)N(R¹²)(R¹³), wherein C₁₋₆alkyl, C₂₋₉heterocycloalkyl, and C₁₋₉heteroaryl are optionally substituted with one, two, or three R^{20b}.

In some embodiments, R² is selected from —OR¹², —N(R¹²)(R¹³), —C(O)R¹⁵, —C(O)N(R¹²)(R¹³);
R¹³ is hydrogen;
R¹² and R¹⁵ are independently

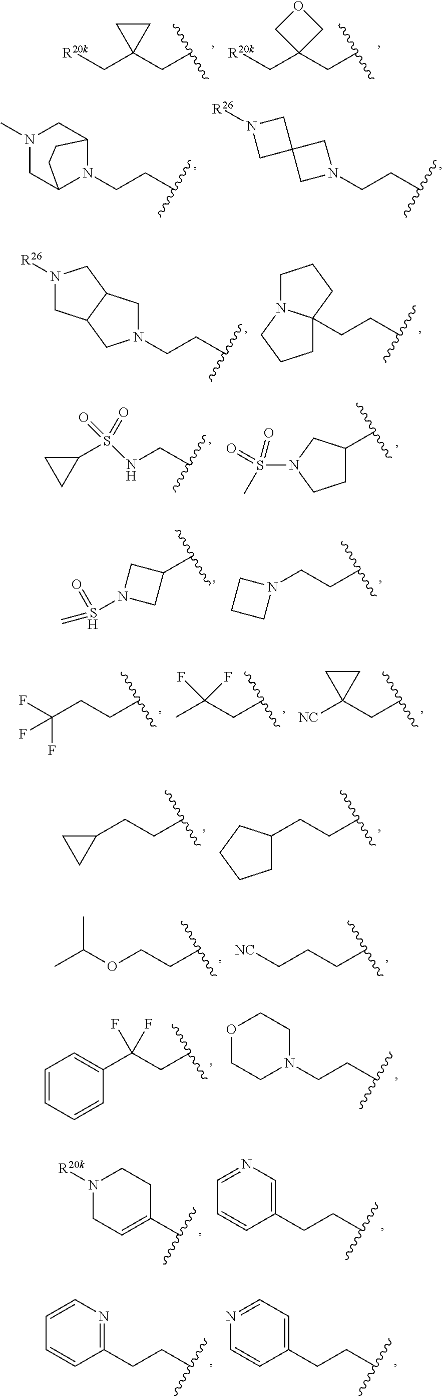

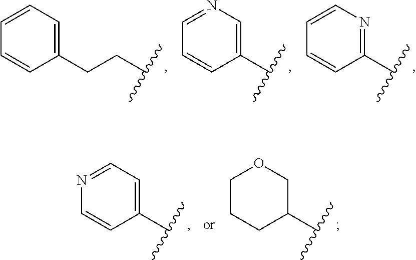

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from —OR¹², —N(R¹²)(R¹³), —C(O)R¹⁵, —C(O)N(R¹²)(R¹³);
R¹³ is hydrogen;
R¹² and R¹⁵ are independently

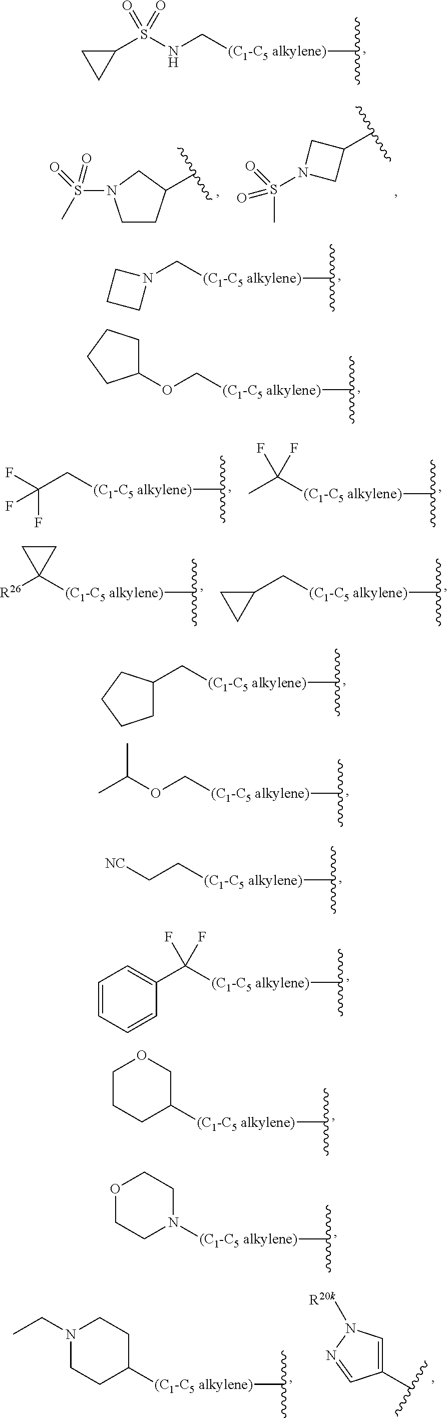

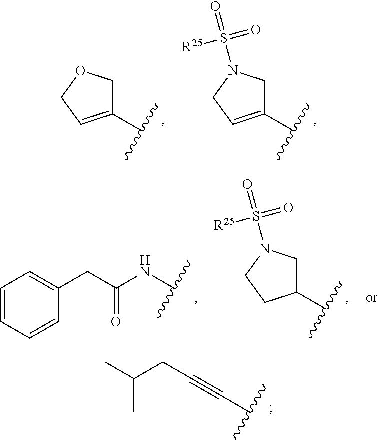

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R² is selected from

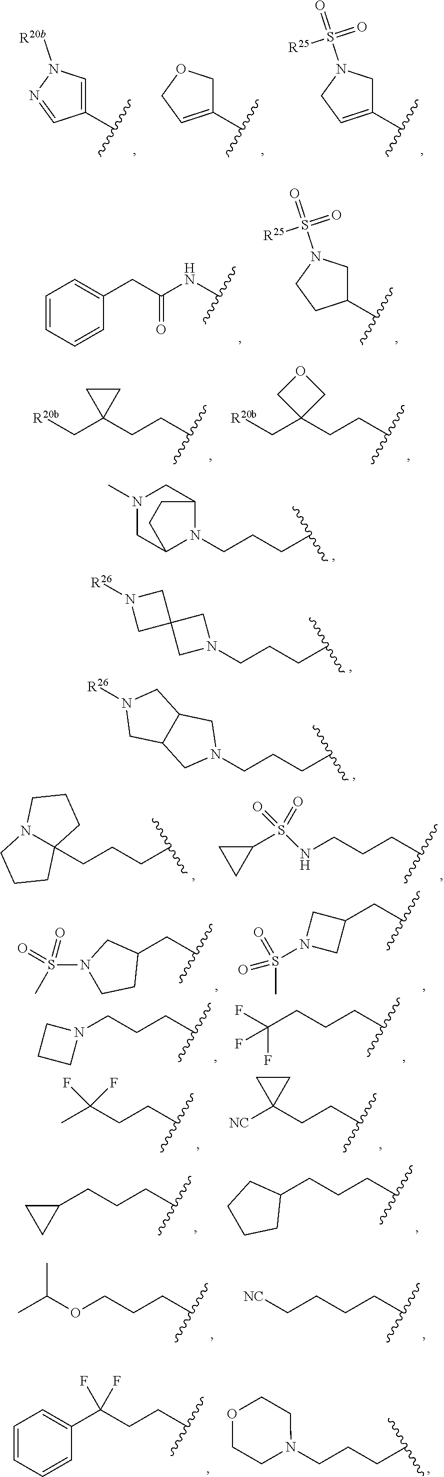

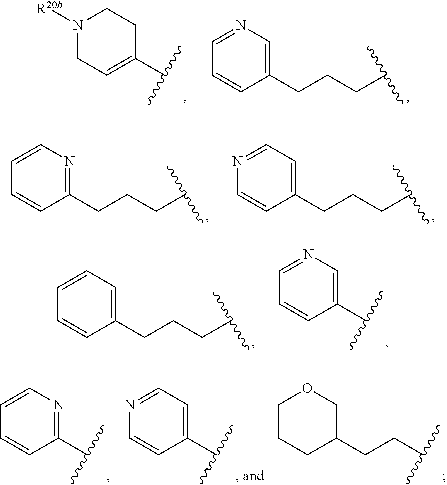

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. n

In some embodiments, R² is independently

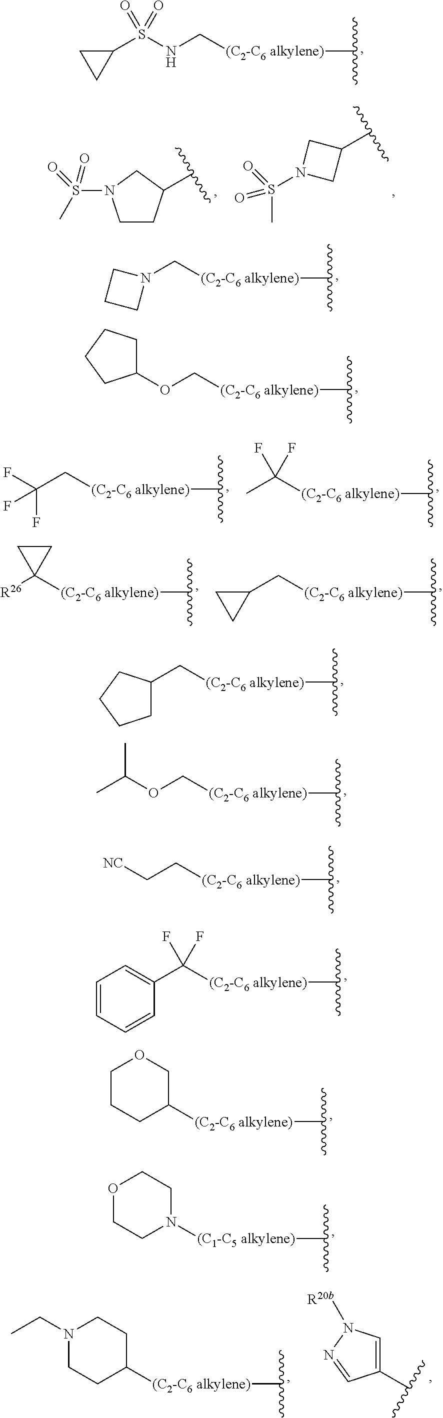

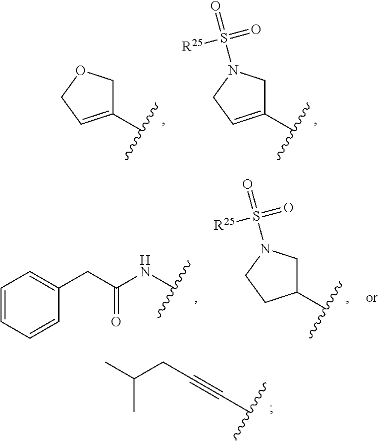

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}.

In some embodiments, R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

Besides the inhibitory effect and high potency in reducing PTPN2 activity, compounds disclosed herein exhibit advantageous bioavailability and exposure compared to another PTPN2 inhibitor. Fine-tuned pharmacological properties embodied in the subject compounds are of great significance for improving efficacy and safety of PTPN2 inhibitors for therapeutic clinical applications.

In some embodiments, compounds of Formula I as well as a sub-formula thereof exhibit at least one or more salient superior pharmacological properties as compared to compounds having a similar core scaffold lacking a core heteroatom. Exemplary superior DMPK properties associated with the subject compounds include but are not limited to increased oral exposure (hence increasing the compounds circulating in a subject's blood upon administration of the compounds), as evidenced by Cmax, AUC, and/or % F.

In some embodiments, a compound of Formula I as well as a sub-formula thereof exhibits an increased oral bioavailability as compared to a reference compound, described in WO2019/246513, having identical substituents on a similar core scaffold that differs from the compounds of Formula I by the absence of the unique and beneficial presence of an additional heteroatom in the core. In an embodiment, such subject compound of Formula I (e.g., Compound A corresponding to Compound No. 154 of Table 6) exhibits an increased oral bioavailability as compared to a compound of WO2019/246513 wherein the reference compound differs from the compound of Formula I solely by the presence of a heteroatom in the core of the compound of Formula I. See Table 4 below that summarizes the results generated utilizing the general procedures in Examples 14 and 15. In an embodiment, such subject compound of Formula I exhibits an increased oral biovailability as compared to a compound of WO2019/246513, wherein the reference compound differs from the compound of Formula I solely by the presence of a nitrogen atom in the core of the compound of Formula I. In some embodiments, a subject compound of Formula I, II, III, IV, XI, XII, XIII, or XIV, or sub-formulae thereof, reveals remarkable improvements in pharmacokinetic properties such as exposure and bioavailability (e.g., particularly oral bioavailability) by at least, 10%, 20%, 30%, 40%, 50%, 100%, 200%, 300%, 400%, or even higher, relative to a reference compound having the same substituents but a different core without an additional core heteroatom. Such superior property attributable to the core heteroatom is present in other subject compounds of the present application, including those of Formula I, II, III, IV, XI, XII, XIII, and XIV, and sub-formulae thereof.

When comparing identical oral dosing of a compound of Formula I (Compound A) against a compound of WO2019/246513 (Reference CMPD below) in a mouse pharmacokinetic model, Compound A displayed a Cmax of 495% that of the Reference CMPD. Furthermore, an AUC_{final} for Compound A was measured to be 356% that of the Reference CMPD measured in the same manner and at the same time in the same mouse model. Finally, the bioavailability (% F) of Compound A was found to be 195% that of the Reference CMPD in the mouse model. When comparing identical oral dosing of a compound of Formula I (Compound A) against a compound of WO2019/246513 (Reference CMPD) in a rat pharmacokinetic model, Compound A displayed a Cmax of 155% that of the Reference CMPD. Furthermore, an AUC_{final} for Compound A was measured to be 224% that of the Reference CMPD measured in the same manner and at the same time in the same rat model. Finally, the bioavailability (% F) of Compound A was found to be 208% that of the Reference CMPD in the rat model. Therefore, the compound of Formula I displayed improved pharmacokinetic characteristics in two different animal models when compared to the Reference CMPD of WO2019/246513 with identical substituents and core structures except for the obligate additional core heteroatom of the compounds of Formula I.

TABLE 4

| | | Compound A vs. Reference CMPD |
|---|---|---|
| | | ((Compound A/Reference CMPD) × 100%) |
| mouse PK PO | Cmax | 495% |
| | AUCₗₐₛₜ | 356% |
| | % F | 195% |
| Rat PK PO | Cmax | 155% |
| | AUCₗₐₛₜ | 224% |
| | % F | 208% |

The above direct comparison (compound pair differs only in the presence of a nitrogen in the core of Compound A of the present application where the reference compound of WO2019/246513 includes a carbon atom) clearly demonstrates that the subject compounds having a core heteroatom exhibit substantially different and superior property in terms of having remarked high exposure as shown by the greater Cmax and AUCₗₐₛₜ in two different animal models along with the approximately two-fold improvement in oral bioavailability as well. These substantially distinct structural and functional attributes of the compounds of the present disclosure are both unexpected and highly beneficially particularly for therapeutic applications.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. In some embodiments, the following synthetic methods may be utilized.

### General Synthetic Method 1

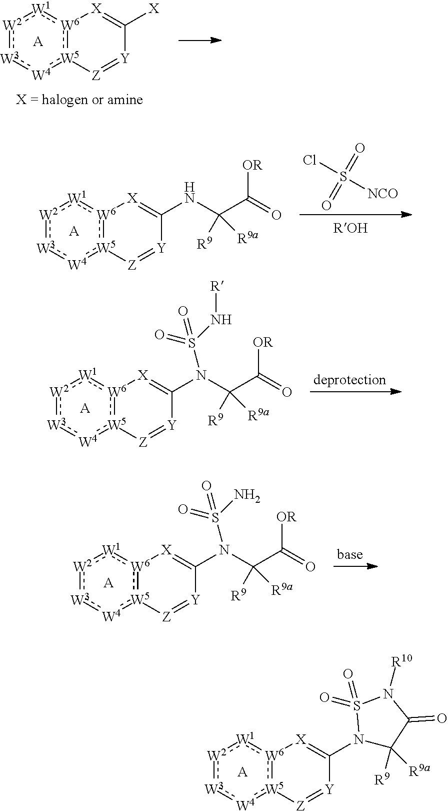

### General Synthetic Method 2

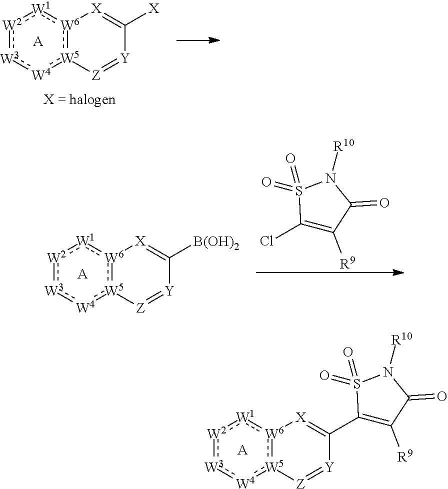

### General Synthetic Method 3

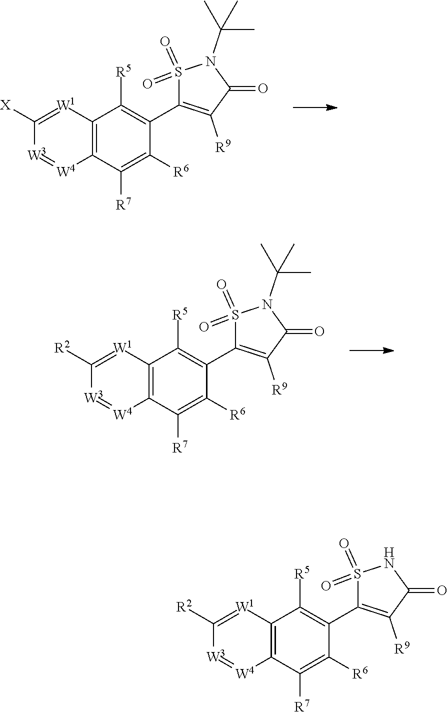

### General Synthetic Method 4

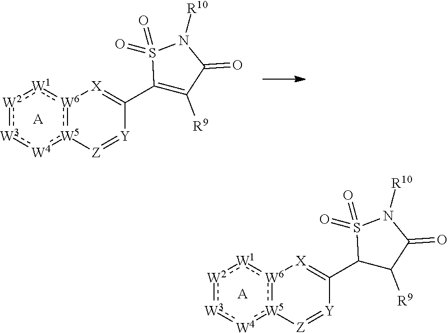

### General Synthetic Method 5

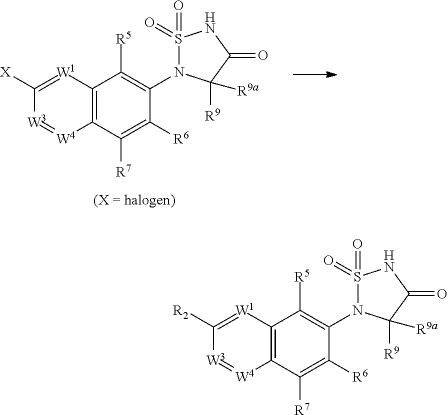

Pharmaceutical Compositions and Methods of Administration

The compounds of compound described herein (e.g., a compound of Formula I, II, III, IV; or a sub-formulae thereof), or a pharmaceutically acceptable salt or solvate thereof, described herein are administered to subjects in a biologically compatible form suitable for administration to treat or prevent diseases, disorders or conditions. Administration of the compounds described herein can be in any pharmacological form including a therapeutically effective amount of a compound described herein (e.g., a compound of Formula I, II, III, IV; or a sub-formulae thereof), or a pharmaceutically acceptable salt or solvate thereof, alone or in combination with a pharmaceutically acceptable carrier.

The compounds of compound described herein (e.g., a compound of Formula XI, XII, XIII, XIV; or a sub-formulae thereof), or a pharmaceutically acceptable salt or solvate thereof, described herein are administered to subjects in a biologically compatible form suitable for administration to treat or prevent diseases, disorders or conditions. Administration of the compounds described herein can be in any pharmacological form including a therapeutically effective amount of a compound described herein (e.g., a compound of Formula XI, XII, XIII, XIV; or a sub-formulae thereof), or a pharmaceutically acceptable salt or solvate thereof, alone or in combination with a pharmaceutically acceptable carrier.

In some embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments is a pharmaceutical composition comprising a pharmaceutically acceptable excipient and a compound described herein (e.g., a compound of Formula I, II, III, IV; or a sub-formulae thereof), or a pharmaceutically acceptable salt or solvate thereof. In some embodiments is a pharmaceutical composition comprising a pharmaceutically acceptable excipient and a compound described herein (e.g., a compound of Formula XI, XII, XIII, XIV; or a sub-formulae thereof), or a pharmaceutically acceptable salt or solvate thereof.

In some embodiments of the methods described herein, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments of the methods described herein, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

### Methods

In an aspect, provided is a method of treating cancer in a subject in need thereof, comprising administering to the subject a therapeutically effective amount of a compound described herein (e.g., a compound of Formula I, II, III, IV; or a sub-formulae thereof), or a pharmaceutically acceptable salt or solvate thereof. In some embodiments, the subject method is applied to treat a solid tumor. In some embodiments, the subject method is applied to treat a hematological cancer. In some embodiments is a method of treating cancer in a subject in need thereof, comprising administering to the subject a therapeutically effective amount of a compound described herein (e.g., a compound of Formula I, II, III, IV; or a sub-formulae thereof), or a pharmaceutically acceptable salt or solvate thereof, wherein the cancer is selected from prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein (e.g., a compound of Formula I, II, III, IV; or a sub-formulae thereof), or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein (e.g., a compound of Formula I, II, III, IV; or a sub-formulae thereof), or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein (e.g., a compound of Formula I, II, III, IV; or a sub-formulae thereof), or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

In an aspect, provided is a method of treating cancer in a subject in need thereof, comprising administering to the subject a therapeutically effective amount of a compound described herein (e.g., a compound of Formula XI, XII, XIII, XIV; or a sub-formulae thereof), or a pharmaceutically acceptable salt or solvate thereof. In some embodiments, this subject method is applied to treat a solid tumor. In some embodiment, this subject method is applied to treat a hematological cancer. In some embodiments, this subject method is provided to treat a cancer selected from prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, this subject method is applied to treat a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein (e.g., a compound of Formula XI, XII, XIII, XIV; or a sub-formulae thereof), or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

In an aspect is provided a method of treating cancer in a subject in need thereof, comprising administering to the subject a therapeutically effective amount of a compound described herein, or a pharmaceutically acceptable salt or solvate thereof.

In an aspect is provided a method of potentiating immunity of a cell, comprising: contacting the cell with a compound described herein, thereby potentiating immunity of the cell, wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.

In an aspect is provided a method of potentiating immunity of a cell, comprising: (a) contacting the cell with a compound described herein; and (b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell. In some embodiments, the cell is a lymphoid cell. In some embodiments, the method further comprises administering the cell to a subject in need thereof. In some embodiments, the method further comprises administering the compound described herein to the subject prior to, concurrent with, or subsequent to the administering the cell.

In some embodiments, prior to the administering the compound described herein, a cell of the subject exhibits expression or activity of PTPN2.

In an aspect is provided a method of potentiating immunity of a subject in need thereof, comprising: (a) selecting the subject that exhibits expression or activity of PTPN2; and (b) downregulating expression or activity of PTPN2 by introducing a compound described herein to a cell of the subject, thereby potentiating immunity of the subject.

In an aspect is provided a method of potentiating immunity of a subject in need thereof, comprising: administering a lymphoid cell to the subject; and administering a compound described herein to the subject, thereby potentiating immunity of the subject.

In an aspect is provided a method of potentiating anti-tumor or anti-cancer immunity of a subject in need thereof, comprising: (a) contacting a lymphoid cell of the subject with a compound described herein, thereby potentiating the anti-tumor or anti-cancer immunity of the subject.

In an aspect is provided a method of treating tumor or cancer of a subject in need thereof, comprising: (a) contacting a lymphoid cell of the subject with a compound described herein, thereby treating the tumor or cancer of the subject.

In an aspect is provided a method of treating tumor or cancer of a subject in need thereof comprising administering to said subject an effective amount of a compound described herein and an additional agent selected from the group consisting of a chemotherapeutic agent and an immune modulator.

In an aspect is provided a method of treating tumor or cancer of a subject in need thereof comprising administering to said subject an effective amount of a compound described herein in conjunction with a cell therapy.

Any of the treatment methods disclosed herein can be administered alone or in combination or in conjunction with another therapy or another agent. By “combination” it is meant to include (a) formulating a subject composition containing a subject compound together with another agent, and (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, chemotherapeutic agents, anti-allergic agents, anti-nausea agents (or anti-emetics), pain relievers, cytoprotective agents, immunostimulants, and combinations thereof.

In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneT), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylnelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In some embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody.

In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7 W, 410C9), anti-B7-113 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg.

In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In combination therapy, a compound provided herein and other anti-cancer agent(s) may be administered either simultaneously, concurrently or sequentially with no specific time limits, wherein such administration provides therapeutically effective levels of the two compounds in the body of the patient.

In some embodiments, a compound of the present disclosure and the other anti-cancer agent(s) are generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases, a body surface area may be utilized to calculate a dosage. A body surface area (BSA) may be calculated by: BSA (m2)=√Height (cm)*Weight (kg)/3600.

### Methods of Reducing Protein Tyrosine Phosphatase Activity

In some embodiments is a method of reducing protein tyrosine phosphatase signalling output, including PTPN2 signaling output. The method typically comprises contacting a PTPN2 protein with an effective amount of a compound described herein (e.g., a compound of Formula I, II, III, IV; or a sub-formulae thereof), or a pharmaceutically acceptable salt or solvate thereof, thereby reducing the PTPN2 signaling output. In some embodiments is a method of reducing PTPN2 signaling output, comprising contacting a PTPN2 protein with an effective amount of a compound described herein (e.g., a compound of Formula I, II, III, IV; or a sub-formulae thereof), or a pharmaceutically acceptable salt or solvate thereof, wherein said the compound inhibits the PTPN2 protein activity or disrupt interaction or binding between a PTPN2 protein and a PTPN2 substrate.

In some embodiments is a method of reducing protein tyrosine phosphatase signalling output, including PTPN2 signaling output. The method typically comprises contacting a PTPN2 protein with an effective amount of a compound described herein (e.g., a compound of Formula XI, XII, XIII, XIV; or a sub-formulae thereof), or a pharmaceutically acceptable salt or solvate thereof, thereby reducing the PTPN2 signaling output. In some embodiments is a method of reducing PTPN2 signaling output, comprising contacting a PTPN2 protein with an effective amount of a compound described herein (e.g., a compound of Formula XI, XII, XIII, XIV; or a sub-formulae thereof), or a pharmaceutically acceptable salt or solvate thereof, wherein said the compound inhibits the PTPN2 protein activity or disrupt interaction or binding between a PTPN2 protein and a PTPN2 substrate.

In some embodiments, provided is a method of reducing PTPN2 signaling output in a cell by contacting the cell with a compound of the present disclosure. In some embodiments, the reduction in PTPN2 activity or signaling output can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8- fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to control untreated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine (i.e. colon)), endometrium, stomach, hematopoietic tissue (e.g., lymphoid tissue), etc.

In some embodiments is a PTPN2 protein bound by a compound described herein (e.g., a compound of Formula I, II, III, IV; or a sub-formulae thereof), or a pharmaceutically acceptable salt or solvate thereof, wherein interaction of PTPN2 protein with a substrate is reduced as compared to a PTPN2 protein unbound to said compound. In some embodiments is a PTPN2 protein bound by a compound described herein (e.g., a compound of Formula XI, XII, XIII, XIV; or a sub-formulae thereof), or a pharmaceutically acceptable salt or solvate thereof, wherein interaction of PTPN2 protein with a substrate is reduced as compared to a PTPN2 protein unbound to said compound.

### Methods of Potentiating Immunity

In an aspect, the present disclosure provides a method of potentiating immunity of a subject in need thereof, comprising administering (e.g., systemically administering) a PTPN2 inhibitor (e.g., compound described herein) to the subject, thereby to potentiate immunity of the subject.

In another aspect, the present disclosure provides a method of potentiating immunity of a subject in need thereof, comprising (e.g., transiently) downregulating expression or activity of PTPN2 in vivo in a cell of the subject, thereby to potentiate immunity of the subject (e.g., comprising administering (e.g., systemically administering) a PTPN2 inhibitor (e.g., compound described herein) to the subject).

In another aspect, the present disclosure provides a method of potentiating immunity of a subject in need thereof, comprising (a) selecting the subject, wherein a cell of the subject exhibits expression or activity of PTPN2; and (b) downregulating the expression or activity of PTPN2 in a cell of the subject, thereby to potentiate immunity of the subject (e.g., comprising administering (e.g., systemically administering) a PTPN2 inhibitor (e.g., compound described herein) to the subject).

In another aspect, the present disclosure provides a method of potentiating immunity of a subject in need thereof, comprising (a) administering a lymphoid cell to the subject, wherein the lymphoid cell comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen; and (b) separately administering a PTPN2 inhibitor (e.g., compound described herein) to the subject, thereby to potentiate immunity of the subject.

In another aspect, the present disclosure provides a method of potentiating immunity of a cell, comprising (a) contacting the cell with a PTPN2 inhibitor (e.g., compound described herein); and (b) introducing to the cell (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen, thereby to potentiate immunity of the cell, wherein (a) is performed prior to or concurrent with (b), thereby to potentiate immunity of the cell.

In another aspect, the present disclosure provides a method of increasing efficacy or reducing side effect of a cell therapy for a subject in need thereof, comprising (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and (b) administering a PTNP2 inhibitor (e.g., compound described herein) to said subject prior to, concurrent with, or subsequent to (a).

In another aspect, the present disclosure provides a method of increasing efficacy or reducing side effect of a cell therapy for a subject in need thereof, comprising (a) administering to the subject a sub-therapeutic amount of a cell comprising a chimeric antigen receptor (CAR) sequence encoding a CAR, and (b) administering a PTNP2 inhibitor (e.g., compound described herein) to said subject prior to, concurrent with, or subsequent to (a).

In practicing any of the methods disclosed herein, a cell or a plurality of such cell may be administered (e.g., systemically administered) to the subject. In some cases, the cell may be a lymphoid cell that optionally comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the cell may be administered (e.g., systemically administered) to the subject sequentially (e.g., prior to or subsequent to) or concurrent with administering (e.g., systemically administering) a PTPN2 inhibitor (e.g., compound described herein) to the subject. The cell may have been contacted previously with a PTPN2 inhibitor (e.g., compound described herein). Alternatively, the cell may not or need not be contacted with a PTPN2 inhibitor (e.g., compound described herein) prior to the administration of the cell to the subject.

In some embodiments, (i) the chimeric T-cell receptor sequence and/or (ii) the CAR sequence may be introduced to the cell directly (e.g., via a solution comprising (i) the chimeric T-cell receptor sequence and/or (ii) the CAR sequence), by chemical means (e.g., via one or more carriers such as liposomes for delivery of one or more nucleic acid sequences comprising (i) the chimeric T-cell receptor sequence and/or (ii) the CAR sequence), and/or viral means (e.g., when delivering one or more nucleic acid sequences comprising (i) the chimeric T-cell receptor sequence and/or (ii) the CAR sequence). For the viral means, the one or more nucleic acid sequence may in introduced in a chromosome of the cell, such as a nuclear chromosome and/or a mitochondrial chromosome. In other embodiments, the one or more nucleic acid sequence may not or need not be introduced in the chromosome of the cell, and as such be introduced to the cell as an epichromosomal molecule (e.g., a linear or circular nucleic acid molecule). In some embodiments, the cell may be a lymphoid cell.

Subsequent to the introduction, (i) the chimeric T-cell receptor sequence and/or (ii) the CAR sequence may persist in the cell for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, or more, or any time in between. Subsequent to the introduction, (i) the chimeric T-cell receptor sequence and/or (ii) the CAR sequence may persist in the cell for at most 5 years, 4 years, 3 years, 24 months, 23 months, 22 months, 21 months, 20 months, 19 months, 18 months, 17 months, 16 months, 15 months, 14 months, 13 months, 12 months, 11 months, 10 months, 9 months, 8 months, 7 months, 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less, or any time in between.

In some embodiments, introducing to the cell (i) the chimeric T-cell receptor sequence and/or (ii) the CAR sequence may be performed sequentially (e.g., prior to or subsequent to) or concurrent with contacting the cell with a PTPN2 inhibitor (e.g., compound described herein). When introduced sequentially, introducing (i) the chimeric T-cell receptor sequence and/or (ii) the CAR sequence and contacting with the PTPN2 inhibitor (e.g., compound described herein) may be performed by the same route (e.g. injections to the same location; tablets taken orally at the same time), or by a different route (e.g. a tablet taken orally while receiving an intravenous infusion). When introduced concurrently, for example, a first composition comprising (i) the chimeric T-cell receptor sequence and/or (ii) the CAR sequence and a second composition comprising the PTPN2 inhibitor (e.g., compound described herein) may be part of the same composition (e.g., the same condition media or a therapeutic regimen).

Contacting the cell with the PTPN2 inhibitor (e.g., compound described herein) (e.g., systemically and/or transiently) as described in the present disclosure, may reduce PTPN2 signaling via reduction of PTPN2 activity or PTPN2 expression in the cell. For example, the cell can be cultured in a suitable medium, to which a PTPN2 inhibitor (e.g., compound described herein) is introduced for period of time sufficient to effect such reduction (or inhibition). Depending on the choice of the type of PTPN2 inhibitor (e.g., compound described herein), the contacting step may be effected by direct physical contact, pressure (e.g. by changing the shape of the cell via squeezing), or chemical means. The PTPN2 inhibitor may directly be introduced to a subject lymphoid cell ex vivo or in vitro. In some embodiments, the cell can be in a subject, and the PTPN2 inhibitor (e.g., compound described herein) may be administered (e.g., systemically administered) to the subject to contact the cell in vivo. Upon such administration, at least a portion of the PTPN2 inhibitor (e.g., compound described herein) may contact a cell (e.g., a lymphoid cell, a cancer, or tumor cell, etc.) of the subject in vivo. A composition (e.g., a therapeutic regimen) comprising the PTPN2 inhibitor (e.g., compound described herein) may be administered to a target site comprising the cell (e.g., the cell may be part of the vascular or lymphatic system of the subject, or a localized tissue of interest or tumor). Alternatively or in addition to, the composition comprising the PTPN2 inhibitor (e.g., compound described herein) may be administered to a different site than the target site. Upon such administration, the PTPN2 inhibitor (e.g., compound described herein) may be directed to the target site or the cell via diffusion or via a medium such as a bodily fluid (e.g., blood).

When contacting a cell (e.g., a lymphoid cell) with the PTPN2 inhibitor (e.g., compound described herein) ex vivo, the cell may be treated with a composition (e.g., a solution) comprising the PTPN2 inhibitor (e.g., compound described herein) for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, or more, or any time in between. The cell may be treated with the composition comprising the PTPN2 inhibitor (e.g., compound described herein) for at most 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or any time in between. During the contacting period, the cell may be subjected to additional PTPN2 inhibitor (e.g., compound described herein) (e.g., to compensate for a limited half-life of the PTPN2 inhibitor (e.g., compound described herein) in culture media).

Alternatively, during the contacting period, the cell may not be subjected to any additional PTPN2 inhibitor (e.g., compound described herein).

A process of contacting the cell with the PTPN2 inhibitor (e.g., compound described herein) (e.g., treating the cell with a composition comprising the PTPN2 inhibitor (e.g., compound described herein)) may be performed at least 1, 2, 3, 4, 5, or more times. In other embodiments, such process may be performed at most 5, 4, 3, 2, or 1 time.

In some embodiments, the cell as provided herein may retain expression or activity of PTPN2 prior to contacting (e.g., in vivo or ex vivo) the cell with the PTPN2 inhibitor (e.g., compound described herein). In some cases, any one of the methods disclosed herein may involve assessing the expression or activity of PTPN2 in the cell prior to contacting the cell with the PTPN2 inhibitor (e.g., compound described herein). In some examples, the cell may not exhibit any loss of the expression or activity of PTPN2, as compared to that present in a control sample, derived from e.g., another cell of the same origin of the cell or a progeny of the cell. In some examples, the cell aberrantly expresses PTPN2 at a level that is above or below a reference level (e.g., a reference level that is considered normal such as non-cancerous). In other examples, the cell may exhibit an expression or activity level of PTPN2 that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In yet some examples, the PTPN2 mRNA level, cDNA level, or PTPN2 polypeptide level expressed in the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, the cell may exhibit an activity level of PTPN2 (e.g., a degree of dephosphorylation of a target substrate) that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, an amount of PTPN2-associated cfDNA or cfRNA level within a source of the cell (e.g., from a plasma of a subject from whom/which the cell was obtained or derived from) may be indicative of an expression level of PTPN2 in the cell. As such, the amount of PTPN2-associated cfDNA or cfRNA level within a source of the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, e.g., another healthy subject who does not comprise or is not suspected of having a condition/disease of interest (e.g., a condition/disease described herein).

For any cell that is administered to a subject in need thereof, either with or without having been treated with a PTPN2 inhibitor (e.g., compound described herein), as provided in the present disclosure, the cell may be autologous or allogenic to the subject. The cell may have been obtained from the subject and treated ex vivo (e.g., contacting with the PTPN2 inhibitor (e.g., compound described herein), engineered to express (i) the TFG and/or (ii) the CAR, etc.) prior to the administration. Alternatively, the cell may be a progeny of a cell obtained from the subject, and the progeny may have been treated ex vivo (e.g., contacting with the PTPN2 inhibitor (e.g., compound described herein), engineered to express (i) the TFG and/or (ii) the CAR, etc.) prior to the administration. In a different alternative, the cell may be a progeny of a cell obtained from the subject, and the progeny may be administered to the subject without any engineering or modification thereof. In other embodiments, the cell may be heterologous to the subject. In some examples, the cell maybe an allogeneic cell, derived from, e.g., another human subject.

Any one of the subject methods disclosed herein may further comprise administering a PTPN2 inhibitor (e.g., compound described herein) to the subject sequentially (e.g., prior to or subsequent to) or concurrent with administering a cell (e.g., a lymphoid cell) to the subject. In some embodiments, the cell may have been at least contacted previously with a PTPN2 inhibitor (e.g., compound described herein) and, optionally, express the TFP and/or the CAR. In other embodiments, the cell may not have been contacted previously with a PTPN2 inhibitor (e.g., compound described herein) and, optionally, express the TFP and/or the CAR. When introduced sequentially, the PTPN2 inhibitor (e.g., compound described herein) and the cell may be administered by the same route (e.g. injections to the same location; tablets taken orally at the same time), or separately by a different route (e.g. a tablet taken orally while receiving an intravenous infusion). When introduced concurrently, the PTPN2 inhibitor (e.g., compound described herein) and the cell may be, e.g., part of the same composition (e.g., the same condition media or a therapeutic regimen).

In some embodiments, a PTPN2 inhibitor (e.g., compound described herein) is administered into a subject in need thereof (e.g., systemically and/or transiently (including intermittently)) to potentiate a subject's immunity. In some embodiments, a PTPN2 inhibitor (e.g., compound described herein) is administered as a single agent. In some embodiments, a PTPN2 inhibitor (e.g., compound described herein) is administered in combination with another agent as a single or unit dose, or as a separate dose. In some embodiments, the another agent can be a cell, including but not limited to a lymphoid cell (e.g., expressing a CAR and/or TCR).

In some embodiments, separate administrations of a cell (e.g., a lymphoid cell optionally configured to express a TFP and/or a CAR) and a PTPN2 inhibitor (e.g., compound described herein) to a subject may occur simultaneously, e.g., administering the cell via a first site of the subject's body and administering the PTPN2 inhibitor (e.g., compound described herein) via a second site of the subject's body at the same time. In other embodiments, separate administrations of the cell and the PTPN2 inhibitor (e.g., compound described herein) may occur sequentially to a same site or to different sites of the subject's body, e.g., administering the PTPN2 inhibitor (e.g., compound described herein) subsequent to the cell, or administering the PTPN2 inhibitor (e.g., compound described herein) prior to the cell. A sequential administration of the cell and the PTPN2 inhibitor (e.g., compound described herein) may be separated by at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, or more, or any time in between. A sequential administration of the cell and the PTPN2 inhibitor (e.g., compound described herein) may be separated by at most 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or any time in between.

In practicing any one of the methods disclosed herein, the subject being administered (e.g., systemically administered) with a PTPN2 inhibitor (e.g., compound described herein) can retain, prior to the administration of the PTPN2 inhibitor (e.g., compound described herein), expression or activity of PTPN2 in the subject's cells, such as lymphoid cells (e.g., T cells, NK cells, HKGY cells, and B cells), cancer cells, or tumor cells. For example, the subject retains a PTPN2 expression or activity level in his or her lymphoid cells, cancer cells, or tumor cells that is at least 10%, 20%, 30%, 40%, 50%, 60%, 70%, 80%, 90%, 95%, 96%, 97%, 98%, 99%, 100% or more of that present in a control sample prior to systemically administering a PTPN2 inhibitor (e.g., compound described herein). In some examples, the PTPN2 mRNA level, cDNA level, PTPN2 or PTPN2-associated cfDNA or cfRNA level, expressed in the subject's lymphoid cells, cancer cells, or tumor cells is at least 10%, 20%, 30%, 40%, 50%, 60%, 70%, 80%, 90%, 95%, 96%, 97%, 98%, 99%, 100% or more of that present in a control sample. In some examples, the PTPN2 mRNA level, cDNA level, PTPN2 or PTPN2-associated cfDNA or cfRNA level, expressed in the subject's lymphoid cells is at least 10%, 20%, 30%, 40%, 50%, 60%, 70%, 80%, 90%, 95%, 96%, 97%, 98%, 99%, 100% or more of that present in a control sample. In some examples, the subject's lymphoid cells, cancer cells, or tumor cells carry two copies or least one copy of PTPN2 genomic DNA. In some examples, the PTPN2 polypeptide level expressed in the subject's lymphoid cells is at least 10%, 20%, 30%, 40%, 50%, 60%, 70%, 80%, 90%, 95%, 96%, 97%, 98%, 99%, 100% or more of that present in a control sample. In some examples, the subject's lymphoid cells, cancer cells, or tumor cells exhibit a normal level of expression or activity of PTPN2 as compared to that of a control sample.

The control sample utilized in assessing the PTPN2 expression level can be a biological sample from a subject that does not exhibit a tumor or cancer, or from a subject that has not been diagnosed with a tumor or cancer and that has not been treated with a PTPN2 inhibitor (e.g., compound described herein). Such control sample can comprise PTPN2 polynucleotides or PTPN2 polypeptides from any of such subject's tissues or cells, including but not limited to such subject's lymphoid cells.

Subsequent to the administration (e.g., systemic administration) of the PTPN2 inhibitor (e.g., compound described herein) to the subject, the subject may exhibit a reduced expression or activity level of PTPN2 in a cell of the subject (e.g., a lymphoid cell, a tumor cell, a cancer cell, etc.) as compared to that present in a control sample from the subject prior to the administration of the PTPN2 inhibitor (e.g., compound described herein). In some cases, subsequent to a systemic administration of the PTPN2 inhibitor (e.g., compound described herein) to the subject, the subject may exhibit at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more reduction in the expression or activity level of PTPN2 in a cell of the subject (e.g., a lymphoid cell, a tumor cell, a cancer cell, etc.) as compared to that present in a control sample from the subject prior to the systemic administration of the PTPN2 inhibitor (e.g., compound described herein). In some cases, the reduced expression or activity level of PTPN2 may be transient, thus may increase over time to, e.g., a normal level comparable to the control sample. In other cases, the reduced expression or activity level of PTPN2 may be maintained or may even continue to decrease for a period of time.

In another aspect, the present disclosure provides a method of potentiating immunity of a subject in need thereof, comprising administering a lymphoid cell (e.g., a T cell, NK cell, HKGY cell, and B cell) to the subject, wherein expression or activity of PTPN2 in the lymphoid cell may be (e.g., transiently) downregulated, thereby potentiating immunity of the subject. In some embodiments, the expression or activity of PTPN2 in the lymphoid cell may be (e.g., transientl)y downregulated without any human intervention (e.g., via a naturally occurring cellular mechanism). In other embodiments, the method herein may further comprise (e.g., transiently) downregulating the expression or activity of PTPN2 in the lymphoid cell.

In practicing any one of the methods disclosed herein, downregulation (e.g., transient downregulation) of PTPN2 expression or activity may be performed in vivo in a cell, such as a lymphoid cell or a diseased cell (e.g., a cancer cell or a tumor cell). In some embodiments, a transient downregulation of expression or activity of a target molecule (e.g., PTPN2) in a cell may involve downregulating the expression or activity of the target molecule for at most about, 6 months, 5 months, 4 months, 3 months, 2 months, 1 month, 21 days, 14 days, 13 days, 12 days, 11 days, 10 days, 9 days, 8 days, 7 days, 6 days, 5 days, 4 days, 3 days, 48 hours, 44 hours, 40 hours, 36 hours, 32 hours, 28 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 55 minutes, 50 minutes, 45 minutes, 40 minutes, 35 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, or a shorter period of time. Subsequent to the transient downregulation, the resulting expression or activity level of the target molecule may be maintained. In other embodiments, subsequent to the transient downregulation, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of the downregulated expression or activity level of the target molecule may be regained.

In practicing any one of the methods disclosed herein, a process of downregulating (e.g., transiently downregulating) expression or activity of a target molecule (e.g., PTPN2) may comprise introducing an inhibitor (e.g., compound described herein) of the target molecule (e.g., a PTPN2 inhibitor (e.g., compound described herein)). In some embodiments, transiently downregulating expression or activity of PTPN2 in a cell (e.g., a lymphoid cell, a tumor cell, a cancer cell) may comprise introducing a PTPN2 inhibitor (e.g., compound described herein) to the cell (e.g., treating the cell with (e.g., a solution comprising) a PTPN2 inhibitor (e.g., compound described herein)) for at most 14 days, 13 days, 12 days, 11 days, 10 days, 9 days, 8 days, 7 days, 6 days, 5 days, 4 days, 3 days, 48 hours, 44 hours, 40 hours, 36 hours, 32 hours, 28 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 55 minutes, 50 minutes, 45 minutes, 40 minutes, 35 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, or a shorter period of time.

In practicing any one of the methods disclosed herein, a cell (e.g., lymphoid cell, a cancer cell, or a tumor cell) of the subject may exhibit expression or activity of PTPN2 (e.g., exhibiting such at a detectable level) before the expression or activity of PTPN2 is downregulated (e.g., transiently downregulated). For example, the cell may exhibit PTPN2 expression or activity level that is at least 10%, 20%, 30%, 40%, 50%, 60%, 70%, 80%, 90%, 95%, 96%, 97%, 98%, 99%, 100% or more of that present in a control sample. In some examples, the PTPN2 mRNA level or cDNA level expressed in the cell is at least 10%, 20%, 30%, 40%, 50%, 60%, 70%, 80%, 90%, 95%, 96%, 97%, 98%, 99%, 100% or more of that present in a control sample. In some examples, the PTPN2 or PTPN2-associated cfDNA or cfRNA level from the cell is at least 10%, 20%, 30%, 40%, 50%, 60%, 70%, 80%, 90%, 95%, 96%, 97%, 98%, 99%, 100% or more of that present in a control sample. In some examples, the cell of interest carries two copies or least one copy of PTPN2 genomic DNA. In some examples, the PTPN2 polypeptide level expressed in the cell is at least 10%, 20%, 30%, 40%, 50%, 60%, 70%, 80%, 90%, 95%, 96%, 97%, 98%, 99%, 100% or more of that present in a control sample. In some examples, the cell exhibits a normal level of expression or activity of PTPN2 as compared to that of a control sample.

The control sample utilized in assessing the PTPN2 expression level in the cell can be a biological sample from a subject that does not exhibit a tumor or cancer, or from a subject that has not been diagnosed with a tumor or cancer and that has not been treated with a PTPN2 inhibitor (e.g., compound described herein). Such control sample can comprise PTPN2 polynucleotides or PTPN2 polypeptides from any of such subject's tissues or cells, including but not limited to such subject's blood plasma.

While expression or activity of PTPN2 in the cell is downregulated (e.g., transiently downregulated), the cell may exhibit a reduced expression or activity level of PTPN2 as compared to that present in the cell prior to the downregulation. In some cases, while expression or activity of PTPN2 in the cell is downregulated (e.g., transiently downregulated), the cell may exhibit at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more reduction in the expression or activity level of PTPN2 as compared to that present in a control sample from the subject prior to the downregulation.

For any one of the subject methods disclosed herein, a process of (e.g., transiently) downregulating the expression or activity of PTPN2 may be performed once. In other embodiments, a process of (e.g., transiently) downregulating the expression or activity of PTPN2 may be performed two or more times. In some cases, the process of (e.g., transiently) downregulating the expression or activity of PTPN2 may be performed intermittently for at least 2, 3, 4, 5, 6, 7, 8, 9, 10, or more times. In some examples, a first (e.g., transiently) downregulation of expression or activity of PTPN2 and a second (e.g., transiently) downregulation of expression or activity of PTPN2 may be separated by period of at least 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 28 hours, 32 hours, 36 hours, 40 hours, 44 hours, 48 hours, 3 days, 4 days, 5 days, 6 days, 7 days, 8 days, 9 days, 10 days, 11 days, 12 days, 13 days, 14 days, or a longer period of time. In other examples, a first (e.g., transiently) downregulation of expression or activity of PTPN2 and a second (e.g., transiently) downregulation of expression or activity of PTPN2 may be separated by period of at most about, 6 months, 5 months, 4 months, 3 months, 2 months, 1 month, 21 days, 14 days, 13 days, 12 days, 11 days, 10 days, 9 days, 8 days, 7 days, 6 days, 5 days, 4 days, 3 days, 48 hours, 44 hours, 40 hours, 36 hours, 32 hours, 28 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 55 minutes, 50 minutes, 45 minutes, 40 minutes, 35 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, or a shorter period of time.

In another aspect, the present disclosure provides a method of potentiating immunity of a subject in need thereof, comprising selecting the subject that exhibits expression or activity of PTPN2. The method further comprises downregulating expression or activity of PTPN2 in a cell of the subject, thereby potentiating immunity of the subject. In some embodiments, the cell of the subject may be a lymphoid cell. Without wishing to be bound by theory, in some embodiments, the cell of the subject may be a tumor cell or a cancer cell.

In some embodiments, (e.g., transiently) downregulating expression or activity of PTPN2 may comprise introducing a PTPN2 inhibitor (e.g., compound described herein) to a cell (e.g., a lymphoid cell, a cancer cell, or a tumor cell) or a subject comprising the same intermittently for two or more times, as provided in the present disclosure. In some examples, a first intermittent dosing regimen of the PTPN2 inhibitor (e.g., compound described herein) and a second intermittent dosing regimen of the PTPN2 inhibitor (e.g., compound described herein) is the same. In yet other examples, a first intermittent dosing regimen of the PTPN2 inhibitor (e.g., compound described herein) and a second intermittent dosing regimen of the PTPN2 inhibitor (e.g., compound described herein) are different. The first intermittent dosing regimen of the PTPN2 inhibitor (e.g., compound described herein) may comprise a PTPN2 inhibitor (e.g., compound described herein) content that is at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, or more than that in the second intermittent dosing regimen. Alternatively, the second intermittent dosing regimen of the PTPN2 inhibitor (e.g., compound described herein) may comprise a PTPN2 inhibitor (e.g., compound described herein) content that is at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, or more than that in the first intermittent dosing regimen. In some examples, the first intermittent dosing regimen and the second intermittent dosing regimen may be administered by the same route (e.g. injections to the same location; tablets taken orally at the same time), or by a different route (e.g. a tablet taken orally while receiving an intravenous infusion).

In practicing any one of the methods disclosed herein, two or more intermittent dosing regimen of a PTPN2 inhibitor (e.g., compound described herein) may be effective to achieve a therapeutically effective plasma concentration of the PTPN2 inhibitor (e.g., compound described herein) in a subject for a duration of time that is substantially the same or longer than that achieved by administering an equivalent dose of the PTPN2 inhibitor (e.g., compound described herein) once (e.g., daily), thereby potentiating immunity of the subject or a cell of the subject (e.g., a lymphoid cell) without causing a side effect. In some cases, a therapeutically effective plasma concentration of a PTPN2 inhibitor (e.g., compound described herein) may be at least about 1 nanomolar (nM), 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 micromolar (μM), 2 μM, 3 μM, 4 μM, 5 μM, 6 μM, 7 μM, 8 μM, 9 μM, 10 μM, or more for a duration of time. In some cases, a therapeutically effective plasma concentration of a PTPN2 inhibitor (e.g., compound described herein) may be at most about 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 21 nM, or less for a duration of time. Such duration of time may be at least about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer.

Any one of the subject methods disclosed herein may further comprise administering a lymphoid cell to the subject sequentially (e.g., prior to or subsequent to) or concurrent with administering a PTPN2 inhibitor (e.g., compound described herein) to the subject. The lymphoid cell may optionally comprise (i) the chimeric T-cell receptor sequence and/or (ii) the CAR sequence. When introduced sequentially, the PTPN2 inhibitor (e.g., compound described herein) and the lymphoid cell may be administered by the same route (e.g. injections to the same location; tablets taken orally at the same time), or by a different route (e.g. a tablet taken orally while receiving an intravenous infusion). When introduced concurrently, the PTPN2 inhibitor (e.g., compound described herein) and the cell may be, e.g., part of the same composition (e.g., the same condition media or a therapeutic regimen). As described elsewhere in the present disclosure, the subject being administered with a PTPN2 inhibitor (e.g., compound described herein) can retain, prior to the administration of the PTPN2 inhibitor (e.g., compound described herein), expression or activity of PTPN2 in the subject's cells, such as lymphoid cells (e.g., T cells, NK cells, HKGY cells, and B cells), cancer cells, or tumor cells.

In practicing any one of the methods disclosed herein, selecting the subject may be based on one or more thresholds of an expression or activity level of PTPN2 in the subject's cells, such as lymphoid cells including, without limitation, effector cells such as T cells, NK cells, HKGY cells, and B cells, cancer cells, or tumor cells. For example, the subject's lymphoid cells, cancer cells, or tumor cells exhibit a PTPN2 expression or activity level in his or her lymphoid cells, cancer cells, or tumor cells that is at least 30%, 40%, 50%, 60%, 70%, 80%, 90%, 95%, or more of that present in a control sample. In some examples, the PTPN2 mRNA level or cDNA level expressed in the subject's lymphoid cells, cancer cells, or tumor cells is at least 30%, 40%, 50%, 60%, 70%, 80%, 90%, 95%, or more of that present in a control sample. In some examples, the PTPN2 or PTPN2-associated cfDNA or cfRNA level from the subject's lymphoid cells, cancer cells, or tumor cells is at least 30%, 40%, 50%, 60%, 70%, 80%, 90%, 95%, or more of that present in a control sample. In some examples, the subject's lymphoid cells, cancer cells, or tumor cells carry two copies or at least one copy of PTPN2 genomic DNA. In some examples, the PTPN2 polypeptide level expressed in the subject's lymphoid cells is at least 30%, 40%, 50%, 60%, 70%, 80%, 90%, 95%, or more of that present in a control sample. In some examples, the subject's lymphoid cells, cancer cells, or tumor cells exhibit a normal level of expression or activity of PTPN2 as compared to that of a control sample. In some cases, selecting the subject that exhibits expression or activity of PTPN2 results in a negative selection against subject that does not express or possess functional PTPN2 as PTPN2-null phenotype, such that the step of downregulating (e.g., transiently downregulating or permanently downregulating) expression or activity of PTPN2 will not be performed.

The control sample utilized in assessing the PTPN2 expression level can be a biological sample from a subject that does not exhibit a tumor or cancer, or from a subject that has not been diagnosed with a tumor or cancer and that has not been treated with a PTPN2 inhibitor (e.g., compound described herein). Such control sample can comprise PTPN2 polynucleotides or PTPN2 polypeptides from any of such subject's tissues or cells, including but not limited to such subject's lymphoid cells.

In some embodiments, downregulating (e.g., transiently downregulating or permanently downregulating) expression or activity of PTPN2 in the cell of the subject may be performed in vivo. In some cases, as described elsewhere in the present disclosure, the cell of the subject may be contacted by a PTPN2 inhibitor (e.g., compound described herein) in vivo by administering the PTPN2 inhibitor (e.g., compound described herein) to the subject comprising the cell. Administering a PTPN2 inhibitor (e.g., compound described herein) to a subject disclosed herein can stimulate or prolong anti-tumor or anti-cancer immunity. In other embodiments, downregulating expression or activity of PTPN2 in the cell of the subject may be performed in vivo. In some cases, as described elsewhere in the present disclosure, the cell of the subject may be isolated from the subject and may be contacted by a PTPN2 inhibitor (e.g., compound described herein) ex vivo, e.g., treated with a composition comprising the PTPN2 inhibitor (e.g., compound described herein).

In practicing any one of the methods disclosed herein, administering a cell (e.g., an autologous or allogeneic lymphoid cell that optionally expresses a TFP and/or a CAR) to the subject may be performed sequentially (e.g., prior to or subsequent to) or concurrent with downregulating (e.g., transiently downregulating or permanently downregulating) expression or activity of PTPN2 in the cell. In some embodiments, the downregulating may comprise introducing a PTPN2 inhibitor (e.g., compound described herein) to the cell, as provided in the present disclosure (e.g., contacting the cell with a PTPN2 inhibitor (e.g., compound described herein). When performed sequentially, a PTPN2 inhibitor (e.g., compound described herein) and the cell may be introduced to the subject by the same route (e.g. injections to the same location; tablets taken orally at the same time), or by a different route (e.g. a tablet taken orally while receiving an intravenous infusion). When performed concurrently, a PTPN2 inhibitor (e.g., compound described herein) and the cell may be, e.g., part of the same composition (e.g., the same condition media or a therapeutic regimen).

In some embodiments, a cell (e.g., a lymphoid cell, a cancer or tumor cell, etc.) of the subject may not exhibit a genetic alteration (e.g., mutation) of (i) a first gene encoding PTPN2 or (ii) a second gene operatively linked to PTPN2, wherein the genetic alteration reduces (or substantially inhibits) the expression and/or activity of PTPN2. In some examples, the second gene may be a promoter operatively linked to PTPN2 or an intron operatively linked to a gene product of PTPN2. Genetic alterations can include a mutation in a polynucleotide (e.g., DNA or RNA) encoding PTPN2 gene product. The mutation can affect any portion of the PTPN2 gene. The one or more PTPN2 mutations can include a mutation in the protein. The one or more PTPN2 mutations can be a point mutation, an insertion, a deletion, an amplification, a translocation, an inversion, or loss of heterozygosity. In some embodiments, the mutation is a loss of function. In some embodiments, the loss of function yields a dominant negative mutation. A mutation can be a frameshift mutation. A frameshift mutation can disrupt the reading frame, resulting in a completely different translated protein as compared to the original sequence. The mutation can be a nonsense mutation. The nonsense mutation can result in a premature stop codon, thus encoding a truncated, and possibly nonfunctional protein product. The PTPN2 mutation can be a nonsense mutation, wherein a single nucleotide alteration causes an amino acid substitution in the translated protein. The mutation can cause an alteration in one or more domain of the PTPN2 protein. The mutation can reduce binding efficacy of a PTPN2 protein with a PTPN2 substrate such as INSR, EGFR, CSF1R, PDGFR, JAK1, JAK2, JAK3, Src family kinases, STAT1, STAT3, STAT6, FYN, LCK, variations thereof, or combinations thereof. The mutation can reduce the ability of PTPN2 to dephosphorylate any one of the substrates disclosed herein, or reduce the ability of PTPN2 to interact with its upstream, or a downstream signaling molecules.

In another aspect, the present disclosure provides a method of potentiating immunity of a subject in need thereof, comprising administering a lymphoid cell to the subject. The method further comprises administering a PTPN2 inhibitor to the subject, thereby potentiating immunity of the subject. In some embodiments, the PTPN2 inhibitor and the lymphoid cell may be administered to the subject in a same composition. In other embodiments, the PTPN2 inhibitor and the lymphoid cell may be administered separately, e.g., in different compositions. In some examples, a PTPN2 inhibitor and a lymphoid cell maybe administered in different compositions via the same route (e.g., separate injections to the same location, different tablets taken orally at the same time), or by a different route (e.g., a tablet taken orally while receiving an intravenous infusion).

In some embodiments, the lymphoid cell may not have been in contact (e.g., treated with) a PTPN2 inhibitor (e.g., ex vivo) prior to being administered to the subject. In other embodiments, the lymphoid cell may have been subjected to a PTPN2 inhibitor (e.g., being contacted with a PTPN2 inhibitor or treated to express a PTPN2 inhibitor) prior to being administered to the subject. In some cases, the lymphoid cell may be subjected to a PTPN2 inhibitor, and the same PTPN2 inhibitor may be administered to the subject. In other cases, the lymphoid cell may be subjected to a PTPN2 inhibitor, and a different PTPN2 inhibitor may be administered to the subject.

As described elsewhere in the resent disclosure, the subject method may further comprise selecting the subject, such that one or more cells of the subject exhibits expression or activity of PTPN2 prior to the administering the PTPN2 inhibitor to the subject. The one or more cells may be lymphoid cells (e.g., T cells, NK cells, HKGY cells, and B cells), cancer cells, or tumor cells.

In another aspect, the present disclosure provides a method of potentiating anti-tumor or anti-cancer immunity of a subject in need thereof, comprising contacting a lymphoid cell of the subject with a PTPN2 inhibitor, thereby potentiating the anti-tumor or anti-cancer immunity of the subject. In a different aspect, the present disclosure provides a method of treating tumor or cancer of a subject in need thereof, comprising contacting a lymphoid cell of the subject with a PTPN2 inhibitor, thereby treating the tumor or cancer of the subject.

In some cases, the lymphoid cell may be contacted with the PTPN2 inhibitor for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, or more, or any time in between. In some cases, the lymphoid cell may be contacted with the PTPN2 inhibitor for at most 14 days, 13 days, 12 days, 11 days, 10 days, 9 days, 8 days, 7 days, 6 days, 5 days, 4 days, 3 days, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or any time in between. During the contacting period, the lymphoid cell may be subjected to additional PTPN2 inhibitor (e.g., to compensate for a limited half-life of the PTPN2 inhibitor in culture media). Alternatively, during the contacting period, the lymphoid cell may not be subjected to any additional PTPN2 inhibitor.

In another aspect, the present disclosure provides a method of potentiating anti-tumor or anti-cancer immunity of a subject in need thereof, comprising downregulating expression or activity of PTPN2 in a lymphoid cell of the subject, thereby potentiating the anti-tumor or anti-cancer immunity of the subject (e.g., comprising administering a compound described herein to the subject in need). In a different aspect, the present disclosure provides a method of treating tumor or cancer of a subject in need thereof, comprising downregulating expression or activity of PTPN2 in a lymphoid cell of the subject, thereby treating the tumor or cancer of the subject (e.g., comprising administering a compound described herein to the subject in need).

In practicing any one of the methods disclosed herein, downregulating the expression or activity of PTPN2 in the lymphoid cell may comprise introducing a PTPN2 inhibitor (e.g., compound described herein) to the lymphoid cell. Introducing the PTPN2 inhibitor may comprise contacting the lymphoid cell with the PTPN2 inhibitor (e.g., compound described herein).

In practicing any one of the methods disclosed herein, the method may further comprise administering the lymphoid cell to the subject sequentially (e.g., prior to or subsequent to) and/or concurrent with the downregulating expression or activity of PTPN2 in the lymphoid cell. In some embodiments, the lymphoid cell may be administered to the subject prior to downregulating expression or activity of PTPN2 in the lymphoid cell. In some examples, downregulating expression or activity of PTPN2 in the lymphoid cell may be performed in vivo, e.g., via introducing a PTPN2 inhibitor (e.g., compound described herein) to the lymphoid cell in vivo. The subject may already comprise the lymphoid cell prior to downregulating the expression or activity of PTPN2 in the lymphoid cell. The lymphoid cell may be an endogenous cell of the subject. Alternatively, the lymphoid cell may be a heterologous lymphoid cell (e.g., an allogeneic cell from a donor or a xenograft cell). In other embodiments, the lymphoid cell may be administered to the subject concurrently with downregulating expression or activity of PTPN2 in the lymphoid cell. In some cases, the lymphoid cell may be subjected to the downregulation of expression or activity of PTPN2 in a composition (e.g., a reaction mixture), and the composition may be administered to the subject. In some examples, the composition may comprise the lymphoid cell and a PTPN2 inhibitor (e.g., compound described herein). The lymphoid cell may be conditioned ex vivo in the composition, in the presence of the PTPN2 inhibitor (e.g., compound described herein), for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, or more, or any time in between. In different embodiments, the lymphoid cell may be administered to the subject subsequent to downregulating expression or activity of PTPN2 in the lymphoid cell. In some examples, the lymphoid cell may be treated ex vivo (e.g., with a PTPN2 inhibitor in an in vitro reaction mixture). Upon confirmation of the downregulation of expression or activity of PTPN2 (e.g., by Western blot or cell activity assay), the lymphoid cell may be administered to the subject. In some examples, to induce downregulation of the expression or activity of PTPN2, the lymphoid cell may be conditioned in vitro, in the presence of the PTPN2 inhibitor (e.g., compound described herein), for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, or more, or any time in between.

In some embodiments, the method may further comprise introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In another aspect, the present disclosure provides a method of increasing efficacy or reducing side effect of a cell therapy for a subject in need thereof, comprising (a) administering to the subject a cell comprising a chimeric antigen receptor (CAR) sequence encoding a CAR. The CAR may comprise an antigen-binding domain and an intracellular signaling domain, wherein the intracellular signaling domain may be minimally required for activation of the CAR upon binding to an antigen. The method further comprises (b) administering a PTNP2 inhibitor (e.g., compound described herein) to the subject prior to, concurrent with, or subsequent to (a). In some embodiments of the subject method, the cell may be a lymphoid cell.

In another aspect, the present disclosure provides a method of increasing efficacy or reducing side effect of a cell therapy for a subject in need thereof, comprising administering to the subject a sub-therapeutic amount of a cell comprising a chimeric antigen receptor (CAR) sequence encoding a CAR. The method further comprises administering a PTNP2 inhibitor (e.g., compound described herein) to the subject prior to, concurrent with, or subsequent to administering the sub-therapeutic amount of the cell to the subject. In some embodiments of the subject method, the cell may be a lymphoid cell. In some embodiments, the cell may retain expression or activity of PTPN2 prior to administering a PTNP2 inhibitor (e.g., compound described herein) to the subject. In some examples, a PTPN2 inhibitor (e.g., compound described herein) may be administered to the subject prior to the administration of the cell, and the cell may be administered and contacted by the PTPN2 inhibitor (e.g., compound described herein) in vivo to effect downregulation of expression or activity of PTPN2 in the cell in vivo. In other examples, a PTPN2 inhibitor (e.g., compound described herein) and the cell may be administered at the same time, e.g., in a same composition or in different compositions, and the cell may be contacted by the PTPN2 inhibitor (e.g., compound described herein) ex vivo and/or in vivo to effect downregulation of expression or activity of PTPN2 in the cell. In different examples, a PTPN2 inhibitor (e.g., compound described herein) may be administered to the subject subsequent to the administration of the cell to the subject, and the cell may be contacted by the PTPN2 inhibitor in vivo to effect downregulation of expression or activity of PTPN2 in the cell in vivo.

A method of potentiating immunity of a subject may comprise administering a lymphoid cell to the subject sequentially (e.g., prior to or subsequent to) and/or concurrent with the downregulation with the PTPN2 inhibitor (e.g., compound described herein). In some embodiments, contacting the lymphoid cell with a PTPN2 inhibitor (e.g., compound described herein) may be performed in vivo, e.g., via administration of the PTPN2 inhibitor (e.g., compound described herein) to the subject. In some cases, the subject may already comprise the lymphoid cell when the PTPN2 inhibitor (e.g., compound described herein) is administered to the subject. The lymphoid cell may be an endogenous cell of the subject. Alternatively, the lymphoid cell may be a heterologous lymphoid cell (e.g., an allogeneic cell from a donor or a xenograft cell). In other cases, the subject may not comprise the lymphoid cell when the PTPN2 inhibitor (e.g., compound described herein) is administered to the subject. Instead, the contact between the PTPN2 inhibitor (e.g., compound described herein) and the lymphoid cell may occur upon administration of the lymphoid cell to the subject subsequent to the administration of the PTPN2 inhibitor (e.g., compound described herein) to the subject. In some embodiments, contacting the lymphoid cell with a PTPN2 inhibitor (e.g., compound described herein) may be performed ex vivo, e.g., in an in vitro culture composition. The lymphoid cell of the subject may be subjected to ex vivo expansion (or cell proliferation) prior to, during, or subsequent to being contacted by the PTPN2 inhibitor (e.g., compound described herein). When the resulting lymphoid cell and/or a progeny thereof is administered to the subject, the lymphoid cell and/or the progeny thereof may be washed to be substantially free of the PTPN2 inhibitor (e.g., compound described herein). Alternatively, the lymphoid cell and/or the progeny may not or need not be washed to rid of any excess, used, or expressed PTPN2 inhibitor (e.g., compound described herein) prior to the administration to the subject.

In some embodiments, the method may further comprise introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the contacting of the lymphoid cell by the PTPN2 inhibitor (e.g., compound described herein) may be performed sequentially (e.g., prior to or subsequent to) or concurrent with the introducing to the lymphoid cell the chimeric T-cell receptor sequence and/or the CAR sequence. In some examples, the lymphoid cell may be contacted with a PTPN2 inhibitor (e.g., compound described herein) prior to being conditioned to express the TFP and/or the CAR. In other examples, the lymphoid cell may be contacted with a PTPN2 inhibitor (e.g., compound described herein) while being conditioned to express the TFP and/or the CAR. In different examples, the lymphoid cell may be configured to express the TFP and/or the CAR prior to being contacted with a PTPN2 inhibitor (e.g., compound described herein).

In some embodiments, the downregulation of the expression or activity of PTPN2 in the lymphoid cell of the subject may be permanent. In other embodiments, as disclosed herein, the downregulation of the expression or activity of PTPN2 in a cell (e.g., the lymphoid cell of the subject) may comprise transiently downregulating the expression or activity of PTPN2.

In some cases, downregulating the expression or activity of PTPN2 in the lymphoid cell performed sequentially (e.g., prior to or subsequent to) or concurrent with the introducing to the lymphoid cell the chimeric T-cell receptor sequence and/or the CAR sequence. In some examples, the expression or activity of PTPN2 in the lymphoid cell may be downregulated (e.g., with a PTPN2 inhibitor (e.g., compound described herein)) prior to being conditioned to express the TFP and/or the CAR. In other examples, the expression or activity of PTPN2 in the lymphoid cell may be downregulated (e.g., with a PTPN2 inhibitor (e.g., compound described herein)) while being conditioned to express the TFP and/or the CAR. In different examples, the lymphoid cell may be configured to express the TFP and/or the CAR prior to downregulating the expression or activity of PTPN2 in the lymphoid cell (e.g., with a PTPN2 inhibitor (e.g., compound described herein)).

In some embodiments, a CAR of the present disclosure contains a minimally required intracellular signaling domain capable of activating a signaling cascade (e.g., an immunoreceptor signaling cascade) of the cell (e.g., in a lymphoid cell) in comparison to a control cell that is (i) without the CAR and/or (ii) in absence of any CAR activation (e.g., in absence of any antigen of the antigen-binding domain of the CAR). A minimally required intracellular signaling domain of the CAR typically consists of a primary signaling domain and lacks a co-stimulatory signaling domain sequence or a functional co-stimulatory signaling domain, and hence exhibiting less potency in activating an immune signaling cascade as compared to one with the co-stimulatory signaling domain. In some examples, the CAR with a minimally required intracellular signaling domain is a first generation CAR. In some examples, the first generation CAR contains only a primary signaling domain selected from the group consisting of CD3zeta, CD28, 4-1BB, OX40, DAP10, ICOS, and a variant thereof. In some examples, the CAR with a minimally required intracellular signaling domain is a second generation CAR. In some examples, the second generation CAR contains only a primary signaling domain selected from the group consisting of CD3zeta, CD28, 4-1BB, OX40, DAP10, ICOS, and a variant thereof, and a co-stimulatory signaling domain that is a different member from the primary signaling domain. In some examples, a cell comprising the CAR with the minimally required intracellular signaling domain may induce a target activity of the cell of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, or more than that of a control cell. In some examples, a cell comprising the CAR with the minimally required intracellular signaling domain may induce a target activity of the cell of at most about 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% or less than that of a control sample comprising a CAR with a more potent intracellular signaling domain. The more potent intracellular signaling domain may comprise a different polypeptide sequence (e.g., a polypeptide fragment derived from a different intracellular protein than the minimally required intracellular signaling domain) or an additional polypeptide sequence (e.g., the minimally required intracellular signaling domain plus one or more additional intracellular signaling domains). The additional polypeptide sequence may comprise at least 1, 2, 3, 4, 5, or more different intracellular signaling domains. Without wishing to be bound by theory, use of a CAR with the minimally required intracellular signaling domain may help to lower toxicity of a cell (e.g., a lymphocyte) expressing the CAR and/or increase persistence of the cell in the body of the subject in need of such cell therapy. In some cases, the use of PTPN2 inhibitor (e.g., compound described herein) in conjunction with CAR-T therapy obviates the need to use other CAR-T cell proliferation inhibitors to control the toxicities inherent in CAR-T therapy. Non-limiting CAR-T cell proliferation inhibitors are specific protein kinase inhibitors such as INSR, EGFR, CSF1R, PDGFR, JAK1, JAK2, JAK3, Src family kinases, STAT1, STAT3, STAT6, FYN, LCK, variations thereof, or combinations thereof. In some embodiments, the methods disclosed herein obviate the need to utilize Nintedanib, Dasatinib, Saracatinib, Ponatinib, Nilotinib, Danusertib, AT9283, Degrasyn, Bafetinib, KW-2449, NVP-BHG712, DCC-2036, GZD824, GNF-2, PD173955, GNF-5, Bosutinib, Gefitinib, Erlotinib, and/or Sunitinib in conjunction of a CAR-T therapy. Another advantage of using PTPN2 inhibitor (e.g., compound described herein) in conjunction with CAR-T therapy is that the amount of CAR-T cells required to yield a comparable level of in vivo efficacy may be reduced. In some cases, a subtherapeutic amount of CAR-T cells is infused into a subject in need thereof. For example, one, two, or three orders of magnitude less of CAR-T cells are needed for treating a subject in need thereof. Where desired, In some embodiments, less than 5×10⁶, 1×10⁶, 5×10⁵, 1×10⁵, 5×10⁴, 1×10⁴ CAR-T cells are needed to yield a comparable level of therapeutic effect as compared to a CAR-T therapy without the use of a PTPN2 inhibitor (e.g., compound described herein).

In practicing any one of the methods disclosed herein, examples of the target activity of the cell may include, but are not limited to, cytokine secretion, gene expression, cell proliferation, cytotoxicity against a target cell, cell death, chemotaxis, cellular metabolism, and/or cell exhaustion.

In practicing any one of the methods disclosed herein, a cell to be administered (e.g., systemically administered) may retain expression or activity of PTPN2 prior to administering a PTPN2 inhibitor (e.g., compound described herein) to the subject. In some examples, a PTPN2 inhibitor (e.g., compound described herein) may be administered to the subject prior to the administration of the cell, and the cell may be administered and contacted by the PTPN2 inhibitor (e.g., compound described herein) in vivo to effect downregulation (e.g., transient downregulation) of expression or activity of PTPN2 in the cell in vivo. In other examples, a PTPN2 inhibitor (e.g., compound described herein) and the cell may be administered at the same time, e.g., in a same composition or in different compositions, and the cell may be contacted by the PTPN2 inhibitor (e.g., compound described herein) ex vivo and/or in vivo to effect downregulation of expression or activity of PTPN2 in the cell. In different examples, a PTPN2 inhibitor (e.g., compound described herein) may be administered to the subject subsequent to the administration of the cell to the subject, and the cell may be contacted by the PTPN2 inhibitor (e.g., compound described herein) in vivo to effect downregulation of expression or activity of PTPN2 in the cell in vivo.

In practicing any one of the methods disclosed herein, a therapeutic amount or an effective amount may be an amount of a composition or a pharmaceutical formulation (e.g., a cell, a PTPN2 inhibitor (e.g., compound described herein), etc.) that is sufficient to elicit a desired response in the subject upon a treatment or method of the present disclosure. In some embodiments, a sub-therapeutic amount of a composition or a pharmaceutical formulation may be an amount of the composition or pharmaceutical formulation that is a fragment of the therapeutic amount. In some examples, a sub-therapeutic amount of a cell (e.g., a cell expression the CAR) may comprise a cell number that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a cell number of a therapeutic amount. For example, one, two, or three orders of magnitude less of CAR-T cells that are normally required absent of the use of PTPN2 inhibitor (e.g., compound described herein) are contemplated for administering into a subject in need thereof. Where desired, In some embodiments, a sub-therapeutic amount of cells such as 5×10⁶, 1×10⁶, 5×10⁵, 1×10⁵, 5×10⁴, or 1×10⁴ CAR-T cells are needed to yield a comparable level of therapeutic effect as compared to a CAR-T therapy without the use of a PTPN2 inhibitor (e.g., compound described herein).

In some examples, a sub-therapeutic amount of a drug (e.g., a PTPN2 inhibitor (e.g., compound described herein)) may comprise a dose of the drug that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a dose of the drug of a therapeutic amount. Without wishing to be bound by theory, use of a sub-therapeutic amount (or dose) of a cell expressing the CAR may help to lower toxicity of such cell therapy and/or increase persistence of the cell in the body of the subject in need of such cell therapy.

In practicing any one of the methods disclosed herein, the immunity of a cell or a subject may be anti-tumor, anti-cancer activity, anti-viral infection activity, and/or anti-bacterial infection activity. In some embodiments, examples of a viral infection and bacterial infection may comprise human bacterial, human parasitic protozoan or human viral infections caused by microbial species including Plasmodium, Pneumocystis, herpes viruses (CMV, HSV 1, HSV 2, VZV, and the like), retroviruses, adenoviruses, and the like. In some examples, any one of the subject methods of the present disclosure may be used to treat or regulate HIV infections and related conditions such as tuberculosis, malaria, Pneumocystis pneumonia, CMV retinitis, AIDS, AIDS-related complex (ARC) and progressive generalized lymphadenopathy (PGL), and AIDS-related neurological conditions such as multiple sclerosis, and tropical spastic paraparesis. Other human retroviral infections that may be treated or regulated by any one of the subject methods of the present disclosure include Human T-cell Lymphotropic virus and HIV-2 infections.

In some embodiments, in practicing any one of the methods disclosed herein, the PTPN2 inhibitor (e.g., compound described herein) does not regulate site-specific recombination of a gene encoding PTPN2. In some examples, the gene encoding PTPN2 or a gene operatively linked to the gene encoding PTPN2 (e.g., a transcription factor, an intron sequence, etc.) may not be flanked by a recombinase site (e.g., Cre recombinase or Flp recombinase substrates). In some examples, the PTPN2 inhibitor (e.g., compound described herein) may not be an activator of recombination of a recombinase site. In an example, the PTPN2 inhibitor (e.g., compound described herein) may not be an estrogen antagonist.

In practicing any one of the methods disclosed herein, the PTPN2 expression or activity level can be determined by detecting the PTPN2 polynucleotides or PTPN2 polypeptides present in a cell or tissue. A wide variety of nucleic acid assays are available for detecting and/or quantifying PTPN2 polynucleotides, including PTPN2 DNAs and PTPN2 RNAs. Exemplary nucleic acid assays include but are not limited to genotyping assays and sequencing methods. Sequencing methods can include next-generation sequencing, targeted sequencing, exome sequencing, whole genome sequencing, massively parallel sequencing, and the like. Several platforms for next generation sequencing are commercially available.

Additional methods for assessing levels and/or concentration of PTPN2 polynucleotides in a tissue or a cell may include, but are not limited to, microarray hybridization assay, nucleic acid amplification assays including without limitation polymerase chain reaction (PCR), quantitative PCR (qPCR), real-time PCR (RT-PCR), digital PCR, and in situ sequencing (US20190024144, US20140349294, incorporated hereby by reference). Nucleic acid amplification can be linear or non-linear (e.g., exponential). Amplification may comprise directed changes in temperature, or may be isothermal. Conditions favorable to the amplification of target sequences by nucleic acid amplification assays are known in the art, can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, some or all of which can be altered.

In situ hybridization (ISH), RNase protection assay, and the like assays can also be employed for detecting PTPN2 polynucleotides and the expression level.

In some embodiments, the copy number PTPN2 gene is assessed by a method selected from the group consisting of in situ hybridization (ISH), Southern blot, immunohistochemistry (IHC), polymerase chain reaction (PCR), quantitative PCR (qPCR), quantitative real-time PCR (qRT-PCR), comparative genomic hybridization (CGH), microarray-based comparative genomic hybridization, and ligase chain reaction (LCR). In some embodiments, the in situ hybridization is selected from fluorescence in situ hybridization (FISH), chromogenic in situ hybridization (CISH) and silver in situ hybridization (SISH). In some embodiments, the copy number is assessed using a nucleic acid sample from the subject, such as genomic DNA, cDNA, ctDNA, cell-free DNA, RNA or mRNA.

PTPN2 expression and/or activity level can also be assessed by detecting and/or quantifying PTPN2 polypeptide level in a subject's tissue or cell. A variety of techniques are available in the art for protein analysis. They include but are not limited to immunohistochemistry (IHC), radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, confocal microscopy, enzymatic assays, surface plasmon resonance and PAGE-SDS. One or more of these protein assays utilizes antibodies or fragments thereof that exhibits specific binding to PTPN2 polypeptides. A large number of anti-PTPN2 antibodies are available, including those provided by Invitrogen, Santa Cruz Biotechnology, OriGene Technologies, MilliporeSigma, Bio-Rad, Abcam, and Cell Signaling Technology. Anti-PTPN2 monoclonal antibodies directed to a given epitope on PTPN2 polypeptide can also be generated by methods known in the art. Such antibodies can be conjugated with an enzyme or other labels for performance of ELISA or other immunoassays described herein. Without wishing to be bound by theory, an anti-PTPN2 antibody may be specific for a functionally active PTPN2, thereby useful for, e.g., selecting a cell or a subject comprising a cell that exhibits expression or activity of PTPN2 above a predetermined threshold level. In other examples, an anti-PTPN2 antibody may be specific for a functionally inactive PTPN2, thereby useful for, e.g., selecting a cell or a subject comprising the cell that expresses PTPN2 variants with genetic alternation(s) that render the protein inactive.

In practicing any one of the subject methods as provided herein, the PTPN2 expression or activity, e.g., in a tumor tissue, a cancer cell, or a lymphoid cell, can be determined using any biological sample comprising the target cells (e.g., plasma cells or cells from a tumor site under investigation) or constituents thereof (e.g., constituents such as cfDNA from the plasma or the tumor site). The biological sample may be a solid or liquid biological sample from the subject under investigation or treatment. The biological sample may be a biopsy sample that is fixed, paraffin-embedded, fresh, or frozen. The biological sample may be obtained by any suitable means, including but not limited to needle aspiration, fine needle aspiration, core needle biopsy, vacuum assisted biopsy, large core biopsy, incisional biopsy, excisional biopsy, punch biopsy, shave biopsy, skin biopsy, and venipuncture.

The biological sample can be obtained from, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues of the subject. In some embodiments, a selection of the biological sample may depend on the condition of the subject to be treated.

In some embodiments, a biological sample comprises cell-free DNA (cfDNA) derived from a whole blood or plasma of the subject. A sample may be analyzed directly for its contents, or may be processed to purify one or more of its contents for analysis. Methods of direct analysis of samples are known in the art and include, without limitation, mass spectrometry and histological staining procedures. In some embodiments, one or more components are purified from the sample for the detection of PTPN2 expression level or activity level. In some embodiments, the purified component of the biological sample is protein (e.g. total protein, cytoplasmic protein, or membrane protein). In some embodiments, the purified component of the sample is a nucleic acid, such as DNA (e.g. genomic DNA, cDNA, ctDNA, or cfDNA) or RNA (e.g. total RNA or mRNA).

Methods for the extraction, purification, and amplification of nucleic acids are known in the art. For example, nucleic acids can be purified by organic extraction with phenol, phenol/chloroform/isoamyl alcohol, or similar formulations, including TRIzol and TriReagent. Other non-limiting examples of extraction techniques include: organic extraction followed by ethanol precipitation, e.g., using a phenol/chloroform organic reagent (Ausubel et al, 1993), with or without the use of an automated nucleic acid extractor, e.g., the Model 341 DNA Extractor available from Applied Biosystems (Foster City, Calif); stationary phase adsorption methods (U.S. Pat. No. 5,234,809; Walsh et al, 1991); and salt-induced nucleic acid precipitation methods (Miller et al, (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristics. In addition to an initial nucleic acid isolation step, purification of nucleic acids can be performed after any step in the methods of the disclosure, such as to remove excess or unwanted reagents, reactants, or products.

In some embodiments, as abovementioned, the cell may be contacted by a PTPN2 inhibitor (e.g., compound described herein) in vivo by administering the PTPN2 inhibitor (e.g., compound described herein) to the subject comprising the cell. Administering a PTPN2 inhibitor (e.g., compound described herein) to a subject disclosed herein can stimulate or prolong anti-tumor or anti-cancer immunity. Not wishing to be bound by any particular theory, a PTPN2 inhibitor (e.g., compound described herein) reduces PTPN2 activity in a cell, leading to an augmented immunoreceptor signaling pathways, which in turn results in the activation of adaptive immunity against tumor or cancer cells.

Stimulation of anti-tumor or anti-cancer immunity can be established by any of the readout known in the art including without limitation: lymphoid cell proliferation (including proliferation of T cells such as CD4+ and/or CD8+ T cells, and clonal expansion other lymphoid cells), cytokine secretion, activation of effector function of lymphoid cells, reduction in T cell exhaustion, destabilization of regulatory T cells (Tregs) and/or their function, movement and/or trafficking of lymphoid cells, release of other intracellular signaling molecules, and phosphorylation of intracellular signaling molecules.

In some embodiments, anti-tumor immunity encompasses proliferation of the lymphoid cells including clonal expansion of the lymphoid cells that are capable of directly or indirectly mediating anti-tumor activity. Non-limiting examples of anti-tumor lymphoid cells are CD4+ and/or CD8+ T cells, NK cells, tumor infiltrating lymphocytes (TIL), especially those T cells capable of specific binding to one or more tumor antigens. Proliferation of the lymphoid cell can lead to a phenotypic change of the lymphoid cell. Treatment of a PTPN2 inhibitor (e.g., compound described herein) can stimulate or prolong lymphoid cell proliferation by about 1 fold, about 2 to about 5 fold, about 5 to about 10 fold, about 10 fold to about 50 fold, about 50 fold to about 100 fold or higher. Assessing lymphoid cell proliferation can be performed by a wide variety of assays known in the art, including without limitation, the use of cell staining, microscopy, flow cytometry, cell sorting, and combinations of these. A number of commercial kits for assessing various types of T cell or B cell proliferations are also suitable to assess the effect of PTPN2 inhibitor (e.g., compound described herein) on T cell or B cell proliferation (e.g., IncuCyte, CellTRrace Cell Proliferation Kits marketed by ThermoFisher). Proliferation can also be determined by phenotypic analysis of the lymphoid cells. For example, clumping of lymphoid cells in culture can signify proliferation of lymphoid cells as compared to comparable lymphoid cells without the treatment with a PTPN2 inhibitor (e.g., compound described herein).

In some embodiments, anti-tumor immunity stimulated or prolonged in response to a PTPN2 inhibitor (e.g., compound described herein) is evidenced by cytokine release from the lymphoid cells. Cytokine release by the lymphoid cell can comprise the release of IFNγ, TNFα, CSF, TGFβ, IL-1, IL-2, IL-4, IL-5, IL-6, IL-13, IL-17, IL-21, IL-22, granzyme, and the like. Lymphoid cells can generate about 1 fold, 2 fold, 3 fold, 4 fold, 5 fold, 6 fold, 7 fold, 8 fold, 9 fold, 10 fold, 50 fold, 100 fold or greater cytokine release in response to a PTPN2 inhibitor (e.g., compound described herein) treatment as compared to comparable lymphoid cells that are not being exposed to the PTPN2 inhibitor (e.g., compound described herein). Cytokine release may be determined and quantified using any immunoassays such as western blot, ELISA, flow cytometry, and the like.

In some embodiments, stimulated or prolonged anti-tumor immunity is evidenced by T cell activation. T cell activation can involve differential expression of antigen specific TCRs, certain cell surface markers and induction of cell proliferation signals. T cell activation may also involve stimulating its effector function including cytolytic activity against tumor or cancer cells, or helper activity including releasing cytokines. In some examples, T cells can be used to kill a tumor or cancer cell in vivo or in vitro in the presence of a PTPN2 inhibitor (e.g., compound described herein). Cell killing can be mediated by the release of one or more cytotoxic cytokines, for example IFNγ or granzyme, by the T cells. In some cases, a subject method can stimulate or prolong the (i) release of cytotoxins such as perforin, granzymes, and granulysin and/or (ii) induction of apoptosis via e.g., Fas-Fas ligand interaction between the T cells and a tumor or cancer cell, thereby triggering the destruction of the target cell. Cytotoxicity can be detected by staining, microscopy, flow cytometry, cell sorting, ELISPOT, chromium release cytotoxicity assay, and other cell death assays described in WO2011131472A1, which is incorporated herein by reference.

Cytotoxicity of a lymphoid cell can be greater in response to treating with a PTPN2 inhibitor (e.g., compound described herein) as compared to a comparable lymphoid cell lacking such treatment. A lymphoid cell treated with a PTPN2 inhibitor (e.g., compound described herein) can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to a comparable lymphoid cell lacking the treatment. In some embodiments, a change in cytotoxicity can comprise comparing such activity before and after treating the lymphoid cell with a PTPN2 inhibitor (e.g., compound described herein).

In some examples, a reduction in expression or activity of such markers including PD1, Foxp3, or Fox03a is indicative of Treg destabilization, and hence an enhanced anti-tumor immunity. In addition, Treg destabilization, as reflected by a decreased T cell exhaustion, can be demonstrated by an enhanced cytokine release, e.g., release of IL-2, IFNγ, TNF and other chemokines.

Anti-tumor immunity can also be evidenced by movement and/or trafficking of the lymphoid cells in response to a treatment with a PTPN2 inhibitor (e.g., compound described herein). In some embodiments, movement can be determined by quantifying localization of the lymphoid cell to a target site such as a tumor tissue. For example, lymphoid cells can be quantified at the target before or after administration of a PTPN2 inhibitor (e.g., compound described herein). Quantification can be performed by isolating a lesion and quantifying a number of lymphoid cells, for example tumor infiltrating lymphocytes. Movement and/or trafficking of lymphoid cells in a tumor tissue after administering a PTPN2 inhibitor (e.g., compound described herein) can be greater than that of a control lacking the administration of a PTPN2 inhibitor (e.g., compound described herein). In some embodiments, the number of lymphoid cells accumulated at the tumor tissue of interest can be about 1 fold, 5 fold, 10 fold, 15 fold, 50 fold, 100 fold or greater than that of a control not being treated with a PTPN2 inhibitor (e.g., compound described herein). Trafficking can also be determined in vitro utilizing a transwell migration assay. In some embodiments, the number of lymphoid cells administered with a PTPN2 inhibitor (e.g., compound described herein) exhibits about 1 fold, 5 fold, 10 fold, 15 fold, 50 fold, 100 fold or greater as compared to that of control lymphoid cells not being administered with a PTPN2 inhibitor (e.g., compound described herein).

Stimulating and/or prolonging anti-tumor immunity in a subject can also be assessed by one or more (in any combination) of the foregoing results, although alternative or additional results of the referenced tests and/or other tests can evidence such desired outcome. In some embodiments, anti-tumor immunity is considered stimulated if there exists at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000%, 10000% or more improvement, using an appropriate measure (e.g. tumor size reduction, duration of tumor size stability, duration of time free from metastatic events, duration of disease-free survival). Improved immunity may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or more, using an appropriate measure (e.g. tumor size reduction, duration of tumor size stability, duration of time free from metastatic events, duration of disease-free survival).

A number of secondary parameters can be employed to determine stimulated and/or prolonged anti-tumor immunity. Examples of secondary parameters include, but are not limited to, the lack of new tumors, a reduction of circulating tumor antigens or markers (e.g., CEA, PSA, CA-125, or cfDNA, ctDNA), the lack of detectable cancer cell or tumor marker by way of biopsy, surgical downstaging (i.e., conversion of the surgical stage of a tumor from unresectable to resectable), MRI, ultrasound, PET scans and any other detection means, all of which can point to the overall immunity to tumor or cancer in a subject. Examples of tumor markers and tumor-associated antigens that can be evaluated as indicators of improved immunity include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, gp100, Ki-67, STK15, Survivin, Cyclin B1, Stromelysin, Cathepsin L2, 3MYBL2, and any ctDNA known in the art. BMC Med. 16:166, 2018.

In some embodiments, prolonged immunity is evidenced by tumor being stabilized (e.g., one or more tumors do not increase more than 1%, 5%, 10%, 15%, or 20% in size, and/or do not metastasize) as a result of treatment with a PTPN2 inhibitor (e.g., compound described herein). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95%, or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

The methods disclosed herein can be applied to treat, stimulate and/or or prolong immunity against a wide variety of cancers, including both solid tumor hematological cancers. For example, the subject methods can be applied to: Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma—see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sézary Syndrome), Ductal Carcinoma In Situ (DCIS), Embryonal Tumors, Endometrial Cancer (Uterine Cancer), Ependymoma, Esophageal Cancer, Esthesioneuroblastoma(Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma(Soft Tissue Sarcoma), Kidney (Renal Cell) Cancer, Laryngeal Cancer (Head and Neck Cancer), Leukemia, Lip and Oral Cavity Cancer(Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma(Skin Cancer), Mesothelioma, Malignant, Metastatic Cancer, Metastatic Squamous Neck Cancer with Occult Primary (Head and Neck Cancer), Midline Tract Carcinoma, Mouth Cancer(Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer(Head and Neck Cancer), Nasopharyngeal Cancer(Head and Neck Cancer), Neuroblastoma, Non-Hodgkin Lymphoma, Non-Small Cell Lung Cancer, Oral Cancer, Lip and Oral Cavity Cancer and Oropharyngeal Cancer(Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma(Soft Tissue Sarcoma), Childhood Vascular Tumors (Soft Tissue Sarcoma), Ewing Sarcoma(Bone Cancer), Kaposi Sarcoma (Soft Tissue Sarcoma), Osteosarcoma(Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and/or any of the aforementioned cancers exhibiting expression and/or activity of PTPN2 in the cancer cells.

In some embodiments contemplate a human subject that has been diagnosed with a cancer, such as one in which PTPN2 expression or activity is detectable (e.g., aberrantly low, normal, or high) in the cancer cells or tumor tissue. Certain other embodiments contemplate a non-human subject, for example a non-human primate such as a macaque, chimpanzee, gorilla, vervet, orangutan, baboon or other non-human primate, including such non-human subjects that can be known to the art as preclinical models, the tumor tissue or cancer cells of which exhibit expression and/or activity of PTPN2. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In some embodiments of the present disclosure, a transgenic animal is utilized. A transgenic animal is a non-human animal in which one or more of the cells of the animal include a nucleic acid that is non-endogenous (i.e., heterologous) and is present as an extrachromosomal element in a portion of its cell or stably integrated into its germ line DNA (i.e., in the genomic sequence of most or all of its cells).

Where desired, the subject can be screened for the presence of expression or activity of PTPN2 in the subject's tumor or cancer cells. The subject can also be screened for the retention of PTPN2 expression and/or activity in one or more types of subject's lymphoid cells. Screening for the presence or the absence of expression or activity of PTPN2 can be carried out by analyzing the PTPN2 polynucleotide or PTPN2 polypeptide with any of the nucleic acid or protein assays disclosed herein. One or more of the screening steps can be performed concurrent with, subsequent to, or more likely, prior to administering a PTPN2 inhibitor (e.g., compound described herein) to the subject.

In some embodiments, one or more steps in the screening, assessment or reporting of the PTPN2 expression and/or activity level is performed with the aid of a processor, such as with a computer system executing instructions contained in computer-readable media. In one aspect, the disclosure provides a system for assessing the PTPN2 expression or activity level in a subject's tumor tissue, cancer cells, and/or the subject's lymphoid cells. In some embodiments, the system comprises (a) a memory unit configured to store information concerning PTPN2 expression and/or activity level present in a tumor tissue/cancer cell, and/or lymphoid cell from the subject being investigated; and (b) one or more processors alone or in combination programmed to (1) assess the PTPN2 expression or activity in the subject's tumor tissue/cancer cell, and/or the PTPN2 expression or activity level in at least one type of subject's lymphoid cells; and (2) assessing the likelihood of a therapeutic beneficial response to treatment with a PTPN2 inhibitor (e.g., compound described herein) based on the presence of the PTPN2 expression or activity in the tumor tissue/cancer cells, and/or the PTPN2 expression or activity in the subject's lymphoid cells.

In some embodiments, a processor or computational algorithm may aid in the assessment of a likelihood of a subject exhibiting a therapeutic benefit to treatment with a PTPN2 inhibitor (e.g., compound described herein). For example, one or more steps of methods or systems described herein may be implemented in hardware, software, firmware where desirable. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A computer system may be involved in one or more of sample collection, sample processing, data analysis, expression profile assessment, calculation of weighted probabilities, calculation of baseline probabilities, comparison of a weighted probability to a reference level and/or control sample, determination of a subject's absolute or increased probability, generating a report, and reporting results to a receiver.

In some embodiments, provided herein is a computer readable medium encoded with computer executable software that includes instructions for a computer to execute functions associated with the identified biomarkers such as PTPN2. Such computer system may include any combination of such codes or computer executable software, depending upon the types of evaluations desired to be completed. The system can have code for calculating a weighted probability of PTPN2 inhibitor (e.g., compound described herein) responsiveness based on the expression and/or activity level present in a subject's tumor tissue or cancer cells, as well as that present in the subject's lymphoid cells.

In a further embodiment, the present disclosure provides a method of treating a cancer, comprising administering an effective amount of a PTPN2 inhibitor (e.g., compound described herein). The PTPN2 inhibitor (e.g., compound described herein) may be effective in one or more of: stimulating and/or prolonging anti-tumor immunity (e.g., destabilizing Tregs, augmenting CD4+ and/or CD8+ T cell function), inhibiting proliferation of cancer cells, inhibiting invasion or metastasis of cancer cells, killing cancer cells, increasing the sensitivity of cancer cells to treatment with a second antitumor agent, and reducing severity or incidence of symptoms associated with the presence of cancer cells. In some embodiments, said method comprises administering to the cancer cells a therapeutically effective amount of a PTPN2 inhibitor (e.g., compound described herein) in vivo. In some embodiments, the administration first takes place ex vivo to a population of effector cells, followed by infusing the PTPN2-inhibitor (e.g., compound described herein) treated effector cells into the subject as further detailed herein. A PTPN2 inhibitor (e.g., compound described herein) compound may be cell-permeable. Alternatively, such compound may not or need not be cell-permeable. A PTPN2 inhibitor (e.g., compound described herein) compound may be reversible, e.g., upon binding and inhibiting activity of PTPN2, it may be released from the PTPN2 to re-activate the PTPN2. Alternatively, such compound may not or need not be reversible.

In some embodiments, the PTPN2 inhibitor (e.g., compound described herein) may not effect editing of (i) a gene encoding PTPN2 or (ii) an additional gene operatively linked to PTPN2 (e.g., transcription factor, intron sequence, start codon, etc.). As such, the gene and/or the additional gene may remain the same upon treatment of a cell with a PTPN2 inhibitor (e.g., compound described herein). In some embodiments, the PTPN2 inhibitor (e.g., compound described herein) may be configured to bind at least a portion of PTPN2. The PTPN2 inhibitor (e.g., compound described herein) may exhibit binding specificity to PTPN2 in comparison to one or more other protein tyrosine phosphatases selected from the group consisting of: PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV, PTPRZ, PTPN1, PTPN2, PTPN3, PTPN4, PTPN5, PTPN6, PTPN7, PTPN9, PTPN11, PTPN12, PTPN13, PTPN14, PTPN18, PTPN20, PTPN21, PTPN23, DUSP1, DUSP2, DUSP4, DUSP5, DUSP6, DUSP7, DUSP8, DUSP9, DUSP10, DUSP16, MK-STYX, DUSP3, DUSP11, DUSP12, DUSP13Aa, DUSP13Ba, DUSP14, DUSP15, DUSP18, DUSP19, DUSP21, DUSP22, DUSP23, DUSP24, DUSP25, DUSP26, DUSP27b, EPM2A, RNGTT, STYX, SSH1, SSH2, SSH3, PTP4A1, PTP4A2, PTP4A3, CDC14A, CDC14B, CDKN3, PTP9Q22, PTEN, TPIP, TPTE, TNS, TENC1, MTM1, MTMR1, MTMR2, MTMR3, MTMR4, MTMR5, MTMR6, MTMR7, MTMR8, MTMR9, MTMR10, MTMR11, MTMR12, MTMR13, MTMR14, MTMR15, ACP1, CDC25A, CDC25B, CDC25C, EYA1, EYA1, EYA1, and EYA1. In some cases, the PTPN2 inhibitor (e.g., compound described herein) may exhibit a half maximal inhibitory concentration (i.e., IC₅₀) of less than or equal to about 10 micromolar (μM), 5 μM, 1 μM, 950 nanomolar (nM), 900 nM, 850 nM, 800 nM, 750 nM, 700 nM, 650 nM, 600 nM, 550 nM, 500 nM, 450 nM, 400 nM, 350 nM, 300 nM, 250 nM, 200 nM, 150 nM, 100 nM, 50 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, or less for PTPN2. The PTPN2 inhibitor (e.g., compound described herein) may exhibit IC₅₀ for PTPN2 that is at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18- fold, 19-fold, 20-fold, 30-fold, 40-fold, 50-fold, 60-fold, 70-fold, 80-fold, 90-fold, 100-fold, or more than that of one or more other protein tyrosine phosphatases. In different embodiments, the PTPN2 inhibitor (e.g., compound described herein) may be configured to bind at least a portion of one or more substrates of PTPN2 selected from the group consisting of: INSR, EGFR, CSF1R, PDGFR, JAK1, JAK2, JAK3, Src family kinases, STAT1, STAT3, STAT6, FYN, LCK, variations thereof, and combinations thereof.

The PTPN2 inhibitor (e.g., compound described herein) may exhibit binding specificity to PTPN2 in comparison to PTPN1B. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 1 nanomolar (nM), 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 micromolar (μM), 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1000 μM, 2 mM, 3 mM, 4 mM, 5 mM, 6 mM, 7 mM, 8 mM, 9 mM, 10 mM, or more. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 1 nanomolar (nM), 2 nM, 3 nM, 4 nM, 5 nM, 6 nM, 7 nM, 8 nM, or 9 nM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 10 nM, 20 nM, 30 nM, 40 nM, 50 nM, 60 nM, 70 nM, 80 nM, or 90 nM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 100 nM, 200 nM, 300 nM, 400 nM, 500 nM, 600 nM, 700 nM, 800 nM, or 900 nM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 1 micromolar (μM), 2 μM, 3 μM, 4 μM, 5 μM, 6 μM, 7 μM, 8 μM, or 9 μM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 10 μM, 20 μM, 30 μM, 40 μM, 50 μM, 60 μM, 70 μM, 80 μM, 90 μM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 100 μM, 200 μM, 300 μM, 400 μM, 500 μM, 600 μM, 700 μM, 800 μM, or 900 μM. In some embodiments, the PTPN2 inhibitor (e.g., compound described herein) may exhibit a half maximal inhibitory concentration (i.e., IC₅₀) for PTPN2 that is lower than the IC50 of the compound for PTPN1B by at least 1 mM, 2 mM, 3 mM, 4 mM, 5 mM, 6 mM, 7 mM, 8 mM, 9 mM, or 10 mM.

The PTPN2 inhibitor (e.g., compound described herein) may exhibit IC₅₀ for PTPN2 that is less than the IC50 of the compound for PTPN1B by a factor of about 2-fold (i.e., if the IC50 for PTPN1B is 100 nM, the IC50 for PTPN2 is about 50 nM), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000-fold, or more. The PTPN2 inhibitor (e.g., compound described herein) may exhibit IC₅₀ for PTPN2 that is less than the IC50 of the compound for PTPN1B by a factor of at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 30-fold, 40-fold, 50-fold, 60-fold, 70-fold, 80-fold, 90- fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000-fold, or more. The PTPN2 inhibitor (e.g., compound described herein) may exhibit IC₅₀ for PTPN2 that is less than the IC50 of the compound for PTPN1B by a factor of at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000-fold, or more.

The PTPN2 inhibitor (e.g., compound described herein) may exhibit IC50 for PTPN2 that is less than the IC50 of the compound for PTPN1B by at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, or 1000%. The PTPN2 inhibitor (e.g., compound described herein) may exhibit IC50 for PTPN2 that is less than the IC50 of the compound for PTPN1B by about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, or 1000%.

The activity or binding of the compound (e.g., compound described herein) to PTPN2 and/or PTPN1B and the relative binding of the compound to PTPN2 compared to PTPN1B may be ascertained by any appropriate assay, for example an assay described herein.

In some embodiments, a PTPN2 inhibitor (e.g., compound described herein) may be conjugated to a degradation tag. A degradation tag may be configured to bind a degradation moiety having a capacity to degrade at least a portion of a target moiety that is bound by the degradation tag. For example, the target moiety is PTPN2 or a substrate of PTPN2. A degradation tag may be a biological or chemical compound, such as a simple or complex organic or inorganic molecule, peptide, peptido mimetic, protein (e.g., antibody), liposome, or a polynucleotide (e.g., small interfering RNA, short hairpin RNA, microRNA, antisense, aptamer, ribozyme, triple helix). Alternatively, a degradation tag may be synthetic. In some cases, any one of the methods described herein may utilize a small molecule degradation tag, and non-limiting examples of such small molecule degradation tag may include, but are not limited to, pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and/or analogs thereof. In some cases, the degradation tag can (i) bind to a degradation moiety such as a ubiquitin ligase (e.g., an E3 ligase such as a cereblon E3 ligase, a VHL E3 ligase, a MDM2 ligase, a TRIM21 ligase, a TRIM24 ligase, and/or a IAP ligase) and/or (ii) serve as a hydrophobic group that leads to protein misfolding of the target moiety, e.g., PTPN2. Misfolding of the target moiety may disrupt activity of the target moiety and/or increase the likelihood of degradation of the target moiety by, e.g., a degradation moiety. In some cases, PTPN2 inhibitor (e.g., compound described herein) may be conjugated to the degradation tag via a linker. Examples of such linker may include, but are not limited to, acyclic or cyclic saturated or unsaturated carbon, ethylene glycol, amide, amino, ether, urea, carbamate, aromatic, heteroaromatic, heterocyclic, and/or carbonyl containing groups with different lengths. Exemplary molecules comprising such degradation tag and method of use thereof are provided in U.S. Patent Publication No. 2019/0336503, which is incorporated herein by reference in its entirety.

In some embodiments, a method of the disclosure provides an effective amount of a PTPN2 inhibitor (e.g., compound described herein). An effective dose refers to an amount sufficient to effect the intended application, including treatment of cancer, stimulating or prolonging anti-tumor immunity. Also contemplated in the subject methods is the use of a sub-therapeutic amount of a PTPN2 inhibitor (e.g., compound described herein) for treating an intended disease or condition.

The amount of the PTPN2 inhibitor (e.g., compound described herein) administered may vary depending upon the intended application (in vitro, ex vivo, or in vivo), or the subject and/or cancer condition being treated, e.g., the weight and age of the subject, the severity of the cancer, the manner of administration and the like.

In some cases, a PTPN2 inhibitor (e.g., compound described herein) may be administered (e.g., systemically administered) to a subject at a dose of at least about 0.1 milligrams per kilogram (mg/kg), 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, or more. In some cases, a PTPN2 inhibitor (e.g., compound described herein) may be administered (e.g., systemically administered) to a subject at a dose of at most about 50 mg/kg, 45 mg/kg, 40 mg/kg, 35 mg/kg, 30 mg/kg, 25 mg/kg, 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, 0.1 mg/kg, or less.

In some cases, upon administration (e.g., systemic administration), a mean plasma concentration of the PTPN2 inhibitor (e.g., compound described herein) in the subject may be at least about 0.1 microgram per milliliter (μg/ml), 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 25 μg/ml, 30 μg/ml, 35 μg/ml, 40 μg/ml, 45 μg/ml, 50 μg/ml, or more. In some cases, upon administration (e.g., systemic administration), a mean plasma concentration of the PTPN2 inhibitor (e.g., compound described herein) in the subject may be at most about 50 μg/ml, 45 μg/ml, 40 μg/ml, 35 μg/ml, 30 μg/ml, 25 μg/ml, 20 μg/ml, 19 μg/ml, 18 μg/ml, 17 μg/ml, 16 μg/ml, 15 μg/ml, 14 μg/ml, 13 μg/ml, 12 μg/ml, 11 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, 0.1 μg/ml, or less.

In some embodiments, a PTPN2 inhibitor (e.g., compound described herein) may be used in combination with another known agent (a second agent) or therapy. Examples of such second agent may be selected from the group consisting of a chemotherapeutic agent, a radioactive agent, anti-cancer agent, a small molecule agent targeting a tumor marker, an antigen-binding agent specifically binding to a tumor marker, and an immune modulator. An immune modulator may be selected from the group consisting of immunostimulatory agents, checkpoint immune blockade agents, and combinations thereof. In some embodiments, the second agent may be a checkpoint inhibitor. In some examples, the second agent may be an inhibitor of PD1, PD-L1, LAG3, CTLA4, CD160, BTLA, LAIR1, TIM3, 2B4, CD93, OX40, Siglec-15, and TIGIT. A PTPN2 inhibitor (e.g., compound described herein) can be administered as part of a therapeutic regimen that comprises administering one or more second agents (e.g. 1, 2, 3, 4, 5, or more second agents), either simultaneously or sequentially with the PTPN2 inhibitor (e.g., compound described herein). When administered sequentially, the PTPN2 inhibitor (e.g., compound described herein) may be administered before, concurrent with, or after the one or more second agents. When administered simultaneously, the PTPN2 inhibitor (e.g., compound described herein) and the one or more second agents may be administered by the same route (e.g. injections to the same location; tablets taken orally at the same time), by a different route (e.g. a tablet taken orally while receiving an intravenous infusion), or as part of the same combination (e.g. a solution comprising the PTPN2 inhibitor (e.g., compound described herein) and one or more second agents). In some examples, a PTPN2 inhibitor (e.g., compound described herein) can be used in combination with a cell therapy, including a TFP- or CAR-expressing cell (e.g., a TFP- or CAR-expressing stem cell or lymphoid cell) described herein. In other examples, a PTPN2 inhibitor (e.g., compound described herein) can be used in combination with a non-cell based therapy, such as surgery, chemotherapy, anti-cancer agent, targeted therapy (e.g., using large or small drug molecules targeting a tumor antigen other than PTPN2), radiation, and the like.

In some embodiments, a PTPN2 inhibitor (e.g., compound described herein) described herein is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by any particular theory, it has been reported that a decrease in L-tryptophan (e.g., catalyzed by IDO) results in an immunosuppressive milieu by inducing T-cell anergy and apoptosis. It is thought that IDO inhibitor can enhance the efficacy of a CAR-expressing cell by decreasing the suppression or death of a CAR-expressing immune cell. While the clinical trial involving the combination of pembrolizumab (an anti-PD1 antibody) and epacadostat (an IDO inhibitor) did not reach the desired end point, a PTPN2 inhibitor (e.g., compound described herein) is expected to potentiate the therapeutic effect of IDO inhibitor. Without being bound by a particular theory, PTPN2 inhibitors (e.g., compound described herein) destabilize the function of the already activated regulatory T cells while the IDO inhibitors prevent the activation of new regulatory T cells. Exemplary inhibitors of IDO that can be used in combination include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255).

Additional agents that can be used in combination with a PTPN2 inhibitor (e.g., compound described herein) include the various categories and examples of agents listed in Table 5 below.

TABLE 5

| | |
|---|---|
| Alkylating agents | Examples include, but are not limited to, altretamine (Hexalen ® or hexamethylmelamine or |
| | HMM), bendamustine, busulfan (Busulfex ® or Myleran ®), carmustine (BiCNU ® or |
| | BCNU ®), chlorambucil, cyclophosphamide (Cytoxan® or Neosar®), dacarbazine (DTIC- |
| | Dome ®), fotemustine, ifosfamide (Ifex ®), improsulfan, lomustine (CCNUR or CeeNU ®), |
| | mechlorethamine or mustine (Mustardgen ®), melphalan (Alkeran ®), nimustine, piposulfan, |
| | ranimustine, semustine, streptozocin (Zanosar ®), temozolomide (Temodar ®), thiotepa |
| | (Thioplex ®), trofosfamide (Ixoten ®), and uramustine or uracil mustard |
| Anthracyclines | Examples include, but are not limited to, aclarubicin, amrubicin, daunorubicin |
| | (Daunomycin ®), daunorubicin (liposomal), doxorubicin (Adriamycin ®), doxorubicin |
| | (liposomal), epirubicin, esorubicin, idarubicin, mitoxantrone, pirarubicin, and valrubicin |
| Anti-angiogenic | Examples include, but are not limited to, aflibercept, axitinib (Inlyta ®), bevacizumab |
| | (Avastin ®), cabozantinib (Cometriq ®), everolimus (Afinitor ® or Zortress ®), lenalidomide |
| | (Revlimid ®), pazopanib (Votrient ®), ponatinib, ramucirumab (Cyramza ®), ranibizumab, |
| | regorafenib (Stivarga ®), sorafenib (Nexavar ®), sunitinib (Sutent ®), thalidomide (Synovir ® |
| | or Thalomid ®) lapatinib, and vandetanib (Caprelsa ®) |
| Antifolates | Examples include, but are not limited to, aminopterin, edatrexate, folic acid, GW1843, |
| | lometrexol, LY309887, methotrexate, nolatrexed, OSI-7904(L), pemetrexed (Alimta ®), |
| | pralatrexate, raltitrexed, trimetrexate, and ZD9331 |
| BCL-2 inhibitors | Examples include, but are not limited to, ABT-199, ABT-263, ABT-737, BH3 mimetics, |
| | gossypol, obatoclax (GX15-070), oblimersen (Genasense ®; G3139; Augmerosen ®), and |
| | venetoclax |
| Bcr-Abl inhibitors | Examples include, but are not limited to, bafetinib (INNO-406), bosutinib (SKI-606), |
| | dasatinib (BMS-354825), imatinib (STI-571), nilotinib (AMN-107), and ponatinib (AP-24534 |
| | or Iclusig ®) |
| Biologics | Examples include, but are not limited to, BCG (TheraCys ®), cytokines (such as INF-alfa, |
| | Aldesleukin, erythropoietin, GM-CSF, G-CSF), gene therapy (such as CT109 or Kymriah ®), |
| | MAbs (such as rituximab, alemtuzumab, ipilimumab, bevacizumab, obinutuzumab, |
| | brentuximab), oncolytic viruses (such as T-VEC or Imlygic ®, H101), and treatment vaccines |
| | (such as sipuleucel-T or Provenge ®) |
| CDK inhibitors | Examples include, but are not limited to, A-674563, abemaciclib (LY2835219), AT-7519, |
| | AZD5438, BMS-265246, BS-181, CYC202 (roscovitine; seliciclib), dinaciclib (SCH- |
| | 727965), flavopiridol (alvocidib), indirubin, JNJ-7706621, K03861, kenpaullone, |
| | LDC000067, LDC4297 (LDC044297), LY2857785, MK-8776 (SCH900776), ML167, |
| | MSC2530818, NU6027, olomoucine, ON123300, P276-00, palbociclib (PD-0332991), |
| | PHA-767491, PHA-793887, PHA-848125 (milciclib), purvalanol A, purvalanol B, R547, |
| | ribociclib (LEE011), RO-3306, senexin A, SNS-032 (BMS-387032), SU9516, TG003, |
| | THZ1, UCN-01 (7-hydroxystaurosporine; KRX-0601), wogonin, and XL413 |
| COX-2 inhibitors | Examples include, but are not limited to, celecoxib (Celebrex ®), etoricoxib, lumiracoxib |
| | (Prexige ®), parecoxib (Dynastat ®), rofecoxib, and valdecoxib (Bextra ®) |
| CTLA-4 inhibitors | Examples include, but are not limited to, ipilimumab (MDX-010) and tremelimumab (10D1) |
| DNA | Examples include, but are not limited to, 1-hydrazinophthalazine, 5,6-dihydro-5-azacytidine, |
| methyltransferase | 5-aza-2′-deoxycytidine, 5-aza-C (5-azacytidine; azacytidine), 5-fluoro-2′-deoxycytidine, |
| inhibitors | arabinosyl-5-azacytidine, decitabine, disulfiram, doxorubicin, EGCG, EGX30P, |
| | hydralazine, MG98, nanaomycin A, nanaomycin C, procainamide, procaine, psammaplin |
| | A, RG108, SGI-1027, sinefungin, thioguanine, and zebularine |
| ERK inhibitors | Examples include, but are not limited to, AG1478, AG99, andrographolide, apigenin, BAY |
| | 43-9006, CAY10561, DEL-22379, ERK inhibitor III, ERK5-IN-1, FR 180204, GDC0994, |
| | GDC-0994, GW5074, hypericin, ISIS 5132, KO947, MK-8353 (SCH900353), ML-9, |
| | PD169316, PD173074, pluripotin, purvalanol, pyrazolylpyrrole ERK inhibitor, SB203580, |
| | SC-1, SCH772984, SL327, SP600125, SU4984, ulixertinib (BVD-523, VRT752271), VX- |
| | 11e (ERK-11e; TCS ERK 11e), and XMD 8-92 |
| Farnesyltransferase | Examples include, but are not limited to, BMS-214662, CP-609754, DK8G557, FTI-277, |
| inhibitors | L744832, L778123, lonafarnib (Sarasar ®; SCH66336), manumycin A, and R115777 |
| | (Zarnestra ®; tipifarnib |
| FLT3 inhibitors | Examples include, but are not limited to, crenolanib besylate (CP-868596-26), gilteritinib |
| | (ASP2215), lestaurtinib (CEP-701), midostaurin (Rydapt ®, PKC412), pexidartinib (PLX3397), |
| | ponatinib (AP24534), quizartinib (AC220), SKLB1028, sorafenib (Nexavar ®), sunitinib |
| | (Sutent ®, SU11248), and XL999 |
| HDAC inhibitors | Examples include, but are not limited to, 4SC-202, Abexinostat (PCI-24781), apicidin, AR- |
| | 42, ATRA, Belinostat (PXD101), BG-45, BRD73954, CAY10603, CG200745, |
| | Chidamide, CHR-2845, CHR-3996, Citarinostat (ACY-241), CUDC-101, CUDC-907, |
| | curcumin, dacinostat (LAQ824), droxinostat, Entinostat (MS-275), FR901228, Givinostat |
| | (ITF2357), HBI-8000, HPOB, ITSA-1, Kevetrin, LAQ824, largazole, LMK-235, M344, |
| | MC-1568, m-carboxycinnamic acid bishydroxamide, ME-344, Mocetinostat (MGCD0103), |
| | nexturastat A, oxamflatin, Panobinostat (LBH589), pracinostat (SB939), pyroxamide, |
| | Quisinostat (JNJ-26481585), Resminostat (4SC-201), RG2833 (RGFP109), RGFP966, |
| | ricolinostat (ACY-1215), Romidepsin (FK228; Depsipeptide), Romidepsin (Istodax), |
| | santacruzamate A (CAY10683), SB939, SBHA, scriptaid, sulforaphane, tacedinaline |
| | (CI994) PCI-34051, tasquinimod, TMP-195, TMP-269, trapoxin A, trichostatin A, tubacin, |
| | tubastatin A, Valproic acid (as Mg valproate), and Vorinostat (SAHA) |
| Hedgehog | Examples include, but are not limited to, AY 9944, BMS-833923 (XL139), GANT58, |
| signaling inhibitors | GANT61, HPI-4, JK184, LEQ 506, PF-04449913, SANT-1, SANT-2, sonidegib (LDE- |
| | 225; Odomzo ®), TAK-441, and vismodegib (IPI-926; Erivedge ®) |
| HIF inhibitors | Examples include, but are not limited to, 2-methoxyestradiol, bortezomib, camptothecin, |
| | echinomycin, ENMD-1198, miltefosine, perifosine, romidepsin, and temsirolimus |
| Hormone therapies | Examples include, but are not limited to, 4(5)-imidazoles, 4-hydroxytamoxifen, |
| | aminoglutethimide, anastrozole (Arimidex ®), bicalutamide megestrol acetate, |
| | diethylstilbestrol, estrace, exemestane (Aromasin ®), fluoxymesterone, flutamide, |
| | fulvestrant (Faslodex ®), goserelin (Zoladex ®), keoxifene, letrozole (Femara ®), leuprorelin, |
| | LY 117018, medroxyprogesterone acetate, nilutamide, octreotide, onapristone, polyestradiol |
| | phosphate, raloxifene, tamoxifen, toremifene, toremifene (Fareston ®), and trioxifene |
| MEK inhibitors | Examples include, but are not limited to, AR-119/RDEA119 (BAY 869766), arctigenin, |
| | ARRY-438162, AS-701173, AS-701255, AS703026, AZD6244 (ARRY-142886), |
| | AZD8330 (ARRY-704), binimetinib (MEK162), CI-1040 (PD184352), E6201, GDC-0623, |
| | GDC-0973 (XL518; cobimetinib), hypothemycin, PD0325901, PD0325901, PD181461, |
| | PD98059, pimasertib, refametinib, RO09-2210, RO4927350, RO4987655, RO5068760, |
| | RO5126766, selumetinib, TAK-733, trametinib (GSK1120212), U0126, and WX-554 |
| Mitotic inhibitors | Examples include, but are not limited to, colchicine, glaziovianin A, griseofulvin, |
| | podophyllotoxin, taxanes, vinblastine, vincristine, vindesine, and vinorelbine |
| mTOR inhibitors - | Examples include, but are not limited to, ABT578, AZD2014, AZD8055, BEZ235, CC-223, |
| immune enhancing | everolimus (RAD001), GSK2126458, INK128 (MLN-0128), Ku-0063794 , LY294002, |
| amount which is | NVP-BEZ235, OSI-027, PI-103, PP242, rapamycin (Sirolimus ®), ridaforolimus |
| typically below the | (Deforolimus ®; AP-23573), temsirolimus (CCI-779), and XL765 |
| therapeutic dose | |
| Multi-kinase | Examples include, but are not limited to, AT9283, dasatinib, lestaurtinib, midostaurin |
| inhibitors | (Rydapt ®), motesanib, neratinib (HKI-272), nilotinib, pazopanib, regorafenib, sorafenib, |
| | sunitinib, vandetanib, and XL184 |
| Nitrogen mustards | Examples include, but are not limited to, bendamustine, chlorambucil, chlornaphazine, |
| | cyclophosphamide, estramustine, ifosfamide, mechlorethamine, mechlorethamine oxide |
| | hydrochloride, melphalan, mustine, novembichin, phenesterine, prednimustine, |
| | trofosfamide, uracil mustard, and uramustine |
| DNA synthesis | Examples include, but are not limited to, 3,4-dihydroxybenzylamine, 5-fluorodeoxyuridine, |
| inhibitors | cladribine, cytarabine, daunorubicin, etoposide, flutarabine, hydroxyurea, idarubicin, |
| | methotrexate, mitoxantrone, and pralatrexate |
| PD-1 inhibitors | Examples include, but are not limited to, AMP-224, AMP-514, atezolizumab (RG7446; |
| | MPDL3280A), BMS-936559, durvalumab, MAB005, MDX-1106, MEDI4736, |
| | MSB0010718C (A09-246-2), nivolumab (BMS936558), pembrolizumab (Lambrolizumab ®; |
| | MK-3475; KEYTRUDA ®), and pidilizumab (CT-011) |
| | Anti PD-1 antibodies: Examples include, but are not limited to, ABCAM ® (AB137132), |
| | AFFYMETRIX EBIOSCIENCE (J105; J116; M1H4), Amplimmune (AMP 514), avelumab |
| | (Bavencio ®), and BIOLEGEND ® (EH12.2H7; RMPI-14) |
| RAF inhibitors | Examples include, but are not limited to, AZ 628, CCT196969, CEP-32496, dabrafenib |
| | (GSK2118436), encorafenib (LGX818), GDC-0879, GW5074, LY3009120, MLN2480, |
| | NVP-BHG712, PLX-4720, PLX-7904, RAF265, RO5126766 (CH5126766), SB590885, |
| | sorafenib tosylate, TAK-632, vemurafenib (PLX4032; RG7204), and ZM 336372 |
| RAS inhibitors | Examples include, but are not limited to, deltarasin, lonafarnib, reolysin, salirasib, siG12D |
| | LODER, SML-8-73-1, tipifarnib, sotorasib (AMG510), adagrasib (MRTX849), MRTX1133, |
| | RMC-6236, RMC-6291, RMC-9805, and RMC-8839. |
| Others | Examples include, but are not limited to, 5-aminolevulinic acid, afatinib, alectinib, |
| | altretamine, anti-CD3 and/or anti-CD33 antibodies (e.g. visilizumab, gemtuzumab, AMG330), |
| | antiproliferative antibodies, aprepitant, arsenic trioxide, benzodopa, biological response |
| | modifiers, bisphosphonates (such as etridonic, clodronic, tiludronic, pamidronic, alendronic, |
| | ibandronic, risedronic, zoledronic acid), bleomycin, blinatumomab (AMG103; Blincyto ®), |
| | brigatinib (Alunbrig ®), buserelin, cabazitaxel, carboquone, ceritinib, crizotinib, |
| | dactinomycin, degarelix, denosumab, dexamethasone, difluoromethylornithine (DFMO), |
| | dronabinol, enasidenib (AG-221), epothilone A or B, eribulin, erlotinib, gefitinib, |
| | gonadorelin agonists, granisetron, heparanase inhibitors, histone deacetylase inhibitors, |
| | histrelin, ibrutinib, inotuzumab ozogamicin (Besponsa ®), ixabepilone, lenvatinib, |
| | leuprolide, levoleucovorin, matrix metalloproteinase inhibitors (such as marimastat (BB- |
| | 2516), prinomastat (AG3340), BMS-279251, BAY 12-9566, TAA211, MMI270B, AAJ996), |
| | methionine aminopeptidase inhibitors, meturedopa, mitomycin, mitotane, nabilone, |
| | necitumumab, niraparib (Zejula ®), olaparib (AZD-2281; Lynparza ®), osimertinib |
| | (Tagrisso ®), palonosetron, panitumumab, pertuzumab, porfimer sodium, revlimid, |
| | thalidomide, tipiracil, trabectedin, trastuzumab, triethylenemelamine, |
| | triethylenephosphoramide, triethylenethiophosphoramide, trimethylolomelamine, and uredopa |

The present disclosure also provides a cell (including a population of cells, such as a population of lymphoid cells) modified to express an exogenous sequence, and wherein expression and/or activity of PTPN2 in said cell has been inhibited (including reduction and elimination). In one aspect, provided in the disclosure is a lymphoid cell in which the expression and/or function of PTPN2 in said cell is inhibited. Such inhibition can be transient or permanent, occurring in vitro, ex vivo, or in vitro. In some cases, as used herein, inhibiting expression and/or function of a target molecule may be referred to downregulation of expression and/or function of the target molecule. A modified lymphoid cell of the present disclosure can be further characterized in that it comprises: (a) a chimeric T-cell receptor sequence encoding a T-cell receptor fusion protein (TFP), and/or (b) a chimeric antigen receptor (CAR) sequence encoding a CAR, wherein each of TFP and CAR exhibits specific binding to an antigen, including but not limited to a tumor or tumor-associated antigen.

Not wishing to be bound by any particular theory, inhibiting PTPN2 expression and/or activity of such lymphoid cell can lead to an augmented immunoreceptor signaling, which in turn results in the activation of an adaptive immunity against tumor or cancer cells. When its PTPN2 expression or activity is inhibited, the modified lymphoid cells can exhibit enhanced cell proliferation (including proliferation of T cells such as CD4+ and/or CD8+ T cells, and clonal expansion other lymphoid cells), enhanced cell activity (including e.g., cytokine secretion, activation of effector function, trafficking to tumor site or cancer cell), or enhanced disability (e.g., reduction in T cell exhaustion, destabilization of regulatory T cells (Tregs) in terms of cell number and cellular function).

In practicing any one of the methods disclosed herein, a subject cell (e.g., a modified cell such as a modified lymphoid cell) may comprise an enhancer moiety capable of enhancing one or more activities of the cell. In some embodiments, an enhancer moiety suitable for incorporating into a subject cell (e.g., a modified lymphoid cell) can be cytokines and growth factors capable of stimulating the growth, clonal expansion, and/or enhancing persistence of the immune cell in vivo. An enhancer may be intracellular, membrane-bound (e.g., a receptor or an adaptor protein of a receptor), or secreted by the cell. Encompassed are enhancer moieties selected from the group consisting of IL-2, IL-3, IL-4, IL-6, IL-7, IL-10, IL-11, IL-12, IL-15, IL-17, IL-18, IL-21, IL-23, PD-1, PD-L1, CD122, CSF1R, CTAL-4, TIM-3, TGFR beta, receptors for the same, functional fragments thereof, functional variants thereof, and combinations thereof. An enhancer moiety may be expressed from an endogenous gene of the cell. Alternatively or in addition to, an enhancer moiety may be expressed from a heterologous gene introduced to the cell. Such heterologous gene may be chromosomal (e.g., in the nuclear chromosome or mitochondrial chromosome) or epichromosomal. In some examples, a cell (e.g., a modified immune cell configured to express a TFP and/or a CAR) may be engineered such that one or more enhancer moieties are constitutively expressed and/or activated. In other examples, the one or more enhancer moieties may be transiently expressed for a limited time. In different examples, the one or more enhancer moieties may be conditionally expressed under, e.g., activation of a cellular signaling.

In practicing any one of the methods disclosed herein, a subject cell (e.g., a modified cell such as a modified lymphoid cell) may comprise an inducible cell death moiety, which inducible cell death moiety effects cell death (e.g., suicide) of the cell upon contact with a cell death activator. Where desired, an inducible cell death moiety is selected from the group consisting of: caspase-1 ICE, caspase-3 YAMA, inducible Caspase 9 (iCasp9), AP1903, HSV-TK, CD19, RQR8, tBID, CD20, truncated EGFR, Fas, FKBP12, CID-binding domain (CBD), and any combination thereof. Examples of further suicide systems include those described by Jones et al. (Jones B S, Lamb L S, Goldman F and Di Stasi A (2014) Improving the safety of cell therapy products by suicide gene transfer. Front. Pharmacol. 5:254. doi: 10.3389/fphar.2014.00254), which is incorporated herein by reference in its entirety. Where desired, a suitable inducible cell death moiety can be HSV-TK, and the cell death activator is GCV. Where further desired, a suitable inducible cell death moiety can be iCasp9, and the cell death activator is AP1903.

A TFP comprised in the subject lymphoid cell typically comprises a TCR subunit comprising (1) a TCR extracellular domain capable of specific binding to an antigen domain, and (2) an intracellular signaling domain. Upon expression of the TFP, it forms a T cell receptor (TCR) complex.

In some embodiments, the TCR extracellular domain comprises (1) an antigen binding domain capable of specific binding to the antigen, and (2) an extracellular domain or portion thereof of a protein including, e.g., the alpha, beta or zeta chain of the T-cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154. In general, the antigen binding domain and the extracellular domain are operatively linked together, e.g., in the same reading frame.

In some embodiments, a subject CAR comprises an antigen-binding domain and an intracellular signaling domain. In some examples, the antigen-binding domain and the intracellular signaling domain of the CAR are linked via a transmembrane domain.

Antigen Binding Domain of TFP or CAR

The antigen binding domain of a TFP or CAR disclosed herein typically comprises an antigen-specific binding element, the choice of which depends upon the type and number of antigen of interest. For example, the antigen binding domain may be chosen to recognize a cell surface marker on a target cell associated with a particular disease state. Non-limiting examples of cell surface markers include those associated tumor or cancer, with viral, bacterial and parasitic infections, autoimmune disease, inflammation diseases and metabolic disease. Cell surface markers can include, without limitation, carbohydrates, glycolipids, glycoproteins; CD (cluster of differentiation) antigens present on cells of a hematopoietic lineage (e.g., CD2, CD4, CD8, CD21, etc.), γ-glutamyltranspeptidase, an adhesion protein (e.g., ICAM-1, ICAM-2, ELAM-1, VCAM-1), hormone, growth factor, cytokine, and other ligand receptors, ion channels, and the membrane-bound form of an immunoglobulin μ. chain. Of particular interest are biological markers associated with a tumor or cancer or a stage or state of a cancer. A vast variety of disease-related biological markers have been identified, and the corresponding targeting moieties have been generated, including but not limited to CD 19, BCMA, CD123, CD38, CD-22, CD33, PSMA, FLT3, cancer antigen-50 (CA-50), cancer antigen-125 (CA-125) associated with ovarian cancer, ERBB2 (Her2/neu), ROR1. Additional disease targets include without limitation TAG72, FAP, CD44v6, CEA, EPCAM, PRSS21, IL-13Ra2, B7H3, KIT, CD30, GD3, CD171, PSCA, VEGFR2, CD24, NCAM, IGF-I receptor, LMP2, fucosyl GM1, GM3, o-acetyl-GD2, CLDN6, TSHR, ALK, polysialic acid, PLAC1, GloboH, NY-BR-1, WT-1, MAGE-A1, sperm protein 17, Tie 2, MAD-CT-2, Fos-related antigen 1, MelanA/MART1, TRP-2, CYP1B1, RAGE-1, intestinal carboxyl esterase, CD79a, CD72, and FCAR. Antibodies comprising CDRs that specifically binds to each of the aforementioned targets are known and/or commercially available. The antigen binding domain of the subject TCR can be generated based on the known CDRs directed to a target of interest.

In still yet some other examples, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

The antigen binding domain can be any domain that binds to the antigen including but not limited to a monoclonal antibody, a polyclonal antibody, a recombinant antibody, a human antibody, a humanized antibody, and a functional fragment thereof, including a Fab, a Fab′, a F(ab′)₂, an Fv, a single chain antibody (e.g., scFv), a minibody, a diabody, a single-domain antibody (“sdAb” or “nanobodies” or “camelids”), or an Fc binding domain.

In some instances, it may beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some instances, the antigen binding domain are “cross-species” in that it binds to the counterpart antigen in a non-human primate, such as Callithrix jacchus, Saguinus oedipus or Saimiri sciureus, in order to facilitate a testing of immunogenicity of the antigen binding domain in these animals.

### Cytoplastic Domain of TFP or CAR

The cytoplasmic domain of the TFP or CAR can include an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in some cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP or CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP or CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

The intracellular signaling sequences within the cytoplasmic portion of the TFP or CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB.

### Transmembrane Domain of TFP or CAR

The extracellular region of TFP or CAR comprising an antigen binding domain can be linked to the intracellular region, for example by a transmembrane domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In one aspect, the transmembrane domain is one that is associated with one of the other domains of the TFP or CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T-cell surface (or another CAR on the CAR-T cell surface). In a different aspect the amino acid sequence of the transmembrane domain may be modified or substituted so as to minimize interactions with the binding domains of the native binding partner present in the same TFP or CAR.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFPor CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154. Where desired, a hinge sequence or linker can be utilized to connect the extracellular domain to the transmembrane domain. Nonlimiting examples of hinge sequences are hinge sequences derived from a human immunoglobulin (Ig) hinge, e.g., an IgG4 hinge, or a CD8a hinge. A variety of linkers, oligo- or polypeptide linker, are available in the art for linking various domains together. They may vary in length from about 2 to 50 amino acids, and vary in amino acid composition. A commonly utilized linker is one enriched in glycine, e.g., amino acid sequence of GGGGSGGGGS (SEQ ID NO. 1), or variations thereof.

In some embodiments, the TFP- or the CAR-expressing cell described herein can further comprise multiple types of TFPs or CARs capable of binding to different antigens, or different epitopes on the same antigen. For instance, a TFP- or CAR-expressing cell of the present disclosure can comprise a second TFP or CAR that includes a different antigen binding domain, e.g., to the same target (CD19 or BCMA) or a different target (e.g., CD123). In one embodiment, when the TFP-expressing cell comprises two or more different TFPs or CARs, the antigen binding domains of the different TFPs or CARs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a VHH. Similarly, a cell expressing a first and second CAR can have an antigen binding domain of the first CAR, e.g., as a fragment, e.g., a scFv, that does not form an association with the antigen binding domain of the second CAR, e.g., the antigen binding domain of the second CAR is a VHH.

In some other embodiments, the TFP- or CAR-expressing cell described herein can further express another agent, e.g., an agent which enhances the activity of a TFP- or CAR-expressing cell. For example, in one embodiment, the agent can be an agent which inhibits an inhibitory molecule. Inhibitory molecules, e.g., PD1, can, in some embodiments, decrease the ability of a TFP- or CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4, CD93, OX40, Siglec-15, and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T-cells and myeloid cells (Agata et al. 1996 Int. Immunol 8:765-75). Two ligands for PD1, PD-L1 and PD-L2 have been shown to downregulate T-cell activation upon binding to PD1 (Freeman et al. 2000 J Exp Med 192:1027-34; Latchman et al. 2001 Nat Immunol 2:261-8; Carter et al. 2002 Eur J Immunol 32:634-43). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-CD19 TFP described herein, improves the persistence of the T-cell. In one embodiment, the TFP or CAR is comprising the extracellular domain of PD 1. Alternatively, provided are TFPs or CARs containing an antibody or antibody fragment such as a scFv that specifically binds to the Programmed Death-Ligand 1 (PD-L1) or Programmed Death-Ligand 2 (PD-L2).

In some embodiments, the present invention provides a population or a mixture of population of TFP- or CAR-expressing cells, in which PTPN2 expression or activity is downregulated (e.g., inhibited). In some examples, the population of TFP-expressing T-cells comprises a mixture of cells expressing different TFPs. The population of TFP-T-cells can include a first cell expressing a TFP having an anti-CD19 or anti-BCMA binding domain described herein, and a second cell expressing a TFP having a different anti-CD19 or anti-BCMA binding domain, e.g., an anti-CD19 or anti-BCMA binding domain described herein that differs from the anti-CD19 binding domain in the TFP expressed by the first cell. As another example, the population of TFP-expressing cells can include a first cell expressing a TFP that includes an anti-CD19 or anti-BCMA binding domain, e.g., as described herein, and a second cell expressing a TFP that includes an antigen binding domain to a target other than CD19 or BCMA (e.g., another tumor-associated antigen). The same approach may apply to a mixture of CAR-expressing cells, individual cells may target the same or different antigens.

Encompassed herein are also additional TFP or CAR configurations known in the art, including Split CARs, RCARs, as well as other TFP and CAR combinations described in WO2016187349, U.S. Pat. No. 9,856,497, WO2017123556, all of which are incorporated herein by reference in their entirety.

Further contemplated are allogeneic CAR-expressing cells in which its expression or activity of PTPN2 is inhibited. For example, the cell can be an allogeneic T cell, e.g., an allogeneic T cell lacking expression of a functional T cell receptor (TCR) and/or human leukocyte antigen (HLA), e.g., HLA class I and/or HLA class II. In particular, a T cell lacking a functional TCR can be, e.g., engineered such that it does not express any functional TCR on its surface, or engineered such that it does not express one or more subunits that comprise a functional TCR, or engineered such that it produces very little functional TCR on its surface. Alternatively, the T cell can express a substantially impaired TCR, e.g., by expression of mutated or truncated forms of one or more of the subunits of the TCR The term “substantially impaired TCR” means that this TCR will not substantially elicit an adverse immune reaction in a host.

Allogeneic T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, a CRISPR system, transcription activator like effector nuclease (TALEN), or zinc finger endonuclease (ZFN).

In some embodiments, an allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a TFP- or CAR-expressing cell to mount an immune effector response. Examples of inhibitory molecules include PD1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4 and TGF beta.

The nucleic acid sequences coding for a desired TFP or CAR can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. Where desired, the TFP- and CAR-expressing cells of the present inventions are generated using lentiviral viral vectors.

Conventional viral and non-viral based gene transfer methods can be used to introduce TFP- or CAR-encoding sequences to a cell of interest, e.g., a lymphoid cell as disclosed herein. Such methods can be used to introduce the TFP- or CAR-encoding sequences to cells in culture, which in turn is administered into a subject. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted sequence. High transduction efficiencies can be observed in many different cell types and target tissues.

A subject lymphoid cell in which its PTPN2 expression and/or activity is downregulated (e.g., inhibited) finds an array of utility in treating a range of diseases associated with the antigen to which the TFP or CAR binds. For instance, PTPN2 downregulation (e.g., inhibition) enhances lymphoid cell expansion, effector function, and survival of human TFP- or CAR-expressing T cells in vitro, and human T cell persistence and antitumor activity in vivo.

In one aspect, the present disclosure provides a method of augmenting activity of an effector cell (e.g., T cells, NK cells, KHYG cells). The method typically comprising: contacting said effector cell with an effective amount of a PTPN2 inhibitor (e.g., compound described herein) such that PTPN2 expression and activity is downregulated (e.g., inhibited) in said effector cell. Augmentation of effector activity can be evidenced by the cytolytic activity against a target cell such as a tumor or cancer cell, or helper activity including the release of cytokines. Assessing augmented effector function can be carried out using any methods known in the art or disclosed here. In some instances, cytotoxicity of an effector cell expressing TFP or CAR as disclosed herein can be greater in response to a PTPN2-inhibitor (e.g., compound described herein) treatment as compared to a control lymphoid cell lacking such treatment. A TFP- or CAR-expressing effector cell treated with a PTPN2 inhibitor (e.g., compound described herein) can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to an effector cell lacking the treatment. In some embodiments, a change in cytotoxicity can comprise comparing such activity before and after treating the effector cell with a PTPN2 inhibitor (e.g., compound described herein). In some other instances, release of cytotoxic cytokines of an effector cell expressing TFP or CAR as disclosed herein can be greater in response to treating with a PTPN2 inhibitor (e.g., compound described herein) as compared to a control lymphoid cell lacking such treatment. Exemplary cytokines include IFNγ, TNFα, CSF, TGFβ, IL-1, IL-2, IL-4, IL-5, IL-6, IL-13, IL-17, IL-21, IL-22, granzyme, and the like. A TFP- or CAR-expressing effector cell can generate about 1 fold, 2 fold, 3 fold, 4 fold, 5 fold, 6 fold, 7 fold, 8 fold, 9 fold, 10 fold, 50 fold, 100 fold or greater release of cytotoxic cytokines in response to a PTPN2 inhibitor (e.g., compound described herein) treatment as compared to a control lymphoid cell that is not being exposed to the PTPN2 inhibitor (e.g., compound described herein).

In another aspect, the present disclosure provides a method of treating cancer in a subject in need thereof, comprising: administering to the subject an effective amount of lymphoid cells, wherein an individual lymphoid cell comprises (a) a chimeric T-cell receptor sequence encoding a T-cell receptor fusion protein (TFP), and/or (b) a chimeric antigen receptor (CAR) sequence encoding a CAR, wherein each of TFP and CAR, when present, exhibits specific binding to an antigen, and wherein expression and/or function of PTPN2 in said cell is downregulated (e.g., inhibited). In some embodiments of the disclosure, downregulation of PTPN2 expression and/or activity can be effected by one or more types of PTPN2 inhibitor (e.g., compound described herein) disclosed herein. Where desired, downregulation of expression or activity of PTPN2 takes place transiently by contacting the cells with a PTPN2 inhibitor (e.g., compound described herein) that asserts such downregulation transiently. Alternatively, PTPN2 downregulation can occur permanently by contacting the cell with a PTPN2 inhibitor (e.g., compound described herein).

In some examples, the practice of the subject method involves downregulating PTPN2 expression and/or activity in the lymphoid cells, ex vivo, prior to administering an effective amount of PTPN2-treated lymphoid cells (e.g., effector cells) to the subject. The ex vivo inhibition can be carried out prior to, concurrent with, or after the introduction of the nucleic acid encoding the TFP or CAR into the lymphoid cell. Such ex vivo treatment may facilitate the expansion and proliferation of the effector cells to yield to a cell count reaching a desired effective amount to be administered to a subject. Such ex vivo treatment may also prolong the survival effector cell persistence and antitumor activity in vivo. For instances, an effector cell of the present invention when infused into a subject is capable of killing tumor or cancer cells in the subject. Unlike antibody therapies, TFP-modified or CAR-modified immune effector cells (e.g., T cells, NK cells, KHYG cells) are able to replicate in vivo resulting in long-term persistence that can lead to sustained tumor control. In various aspects, the immune effector cells (e.g., T cells, NK cells, KHYG cells) administered to the subject, or their progeny, persist in the subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell or KHYG cells to the subject.

Accordingly, the present disclosure also provides a method of increasing the therapeutic efficacy of a TFP- or CAR-expressing cell directed to a tumor or tumor associated antigen. In some embodiments, administering a PTPN2 inhibitor (e.g., compound described herein) occurs ex vivo. In other embodiments, administering a PTPN2 inhibitor (e.g., compound described herein) occurs in vivo prior to, concurrent with, or after the cells have been administered to a subject, where the cell may have or may not have previously been exposed to the PTPN2 inhibitor (e.g., compound described herein) ex vivo.

In one aspect, a fully-human TFP- or CAR-modified immune effector cells (e.g., T cells, NK cells, KHGY cells) of the invention may be a type of vaccine for ex vivo immunization and/or in vivo therapy in a mammal including a human.

The subject methods utilizing a TFP- or CAR-expressing lymphoid cells (including e.g., effector cells) that target one or more tumor antigens can be applied to treat solid tumor and hematological cancers disclosed herein.

The present disclosure also provides a pharmaceutical compositions comprising a TFP- or CAR-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity of administration will be determined by such factors as the condition of the subject, and the type and severity of the subject's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenesfaecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The precise effective amount of the compositions of the present invention to be administered can be determined by a physician with consideration of individual differences in age, weight, tumor size, extent of infection or metastasis, and condition of the subject. It can generally be stated that a pharmaceutical composition comprising the T-cells described herein may be administered at a dosage of 10⁴ to 10⁹ cells/kg body weight, in some instances 10⁵ to 10⁶ cells/kg body weight, including all integer values within those ranges. T-cell compositions may also be administered multiple times at these dosages. The cells can be administered by using infusion techniques that are commonly known in immunotherapy (see, e.g., Rosenberg et al., New Eng. J. of Med. 319:1676, 1988).

In some examples, it may be desired to administer activated T-cells to a subject and then subsequently redraw blood (or have an apheresis performed), activate T-cells therefrom according to the present invention, and reinfuse the patient with these activated and expanded T-cells. This process can be carried out multiple times every few weeks. In certain aspects, T-cells can be activated from blood draws of from 10 cc to 400 cc. In certain aspects, T-cells are activated from blood draws of 20 cc, 30 cc, 40 cc, 50 cc, 60 cc, 70 cc, 80 cc, 90 cc, or 100 cc. In some embodiments, the T-cell compositions of the present invention are administered by i.v. injection. The compositions of T-cells may be injected directly into a tumor, lymph node, or site of infection.

In some examples, a subject may undergo leukapheresis, wherein leukocytes are collected, enriched, or depleted ex vivo to select and/or isolate the cells of interest, e.g., T-cells. These T-cell isolates may be expanded by methods known in the art and treated such that one or more TFP constructs of the invention may be introduced, thereby creating a TFP-expressing or CAR-expressing T-cell of the invention.

### ADDITIONAL EMBODIMENTS

EMBODIMENT 1. A compound of Formula (I), or a pharmaceutically acceptable salt or solvate thereof:

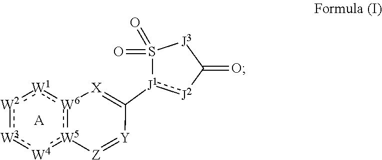

wherein:
W¹ is N, N(R¹), O, C(R¹), C(R¹)(R^{1a}), C(O), S(O), S(O)₂, S(O)(NR¹), S(O)(R¹), or P(O)(R¹);
W² is N, N(R²), O, C(R²), C(R²)(R^{2a}), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein at least one of W¹, W², W³, W⁴, W⁵, and W⁶ is N, N(R¹), N(R²), N(R³), N(R⁴), or O; and
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁_.heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
R^{12a} is selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k}.
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁_.heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁_.heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and
provided that:
at least two of Ring A [symbol] are double bonds;
when J³ is CH₂; R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁_.heteroaryl, —OR¹⁶, —N(R¹²)(R¹⁶), —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f}; and
the compound is not

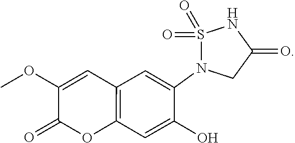 [SMILES: COc1cc2cc(N3CC(=O)NS3(=O)=O)c(O)cc2oc1=O]

EMBODIMENT 2. The compound of Embodiment 1, or a pharmaceutically acceptable salt or solvate thereof, wherein X is C(R⁵).
EMBODIMENT 3. The compound of Embodiment 1 or Embodiment 2, or a pharmaceutically acceptable salt or solvate thereof, wherein Y is C(R⁶).
EMBODIMENT 4. The compound of any one of Embodiments 1-3, or a pharmaceutically acceptable salt or solvate thereof, wherein Z is C(R⁷).
EMBODIMENT 5. The compound of any one of Embodiments 1-4, or a pharmaceutically acceptable salt or solvate thereof, wherein J¹ is N and J² is C(R⁹)(R^{9a})
EMBODIMENT 6. The compound of any one of Embodiments 1-4, or a pharmaceutically acceptable salt or solvate thereof, wherein J¹ is C(R⁸) and J² is C(R⁹)(R^{9a})
EMBODIMENT 7. The compound of any one of Embodiments 1-6, or a pharmaceutically acceptable salt or solvate thereof, wherein J² is CH₂.
EMBODIMENT 8. The compound of any one of Embodiments 1-4, or a pharmaceutically acceptable salt or solvate thereof, wherein J¹ is C and J² is C(R⁹).
EMBODIMENT 9. The compound of Embodiment 8, or a pharmaceutically acceptable salt or solvate thereof, wherein J² is C(H).
EMBODIMENT 10. The compound of any one of Embodiments 1-9, or a pharmaceutically acceptable salt or solvate thereof, wherein J³ is N(R¹⁰).
EMBODIMENT 11. The compound of any one of Embodiments 1-10, or a pharmaceutically acceptable salt or solvate thereof, wherein J³ is N(H).
EMBODIMENT 12. The compound of any one of Embodiments 1-4, or a pharmaceutically acceptable salt or solvate thereof, having the structure of Formula (Ia):

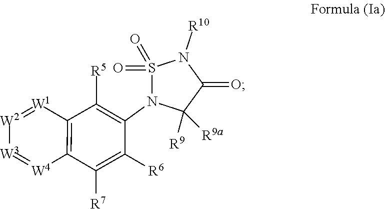

wherein at least one of W¹, W², W³, and W⁴ is N.
EMBODIMENT 13. The compound of any one of Embodiments 1-4, or a pharmaceutically acceptable salt or solvate thereof, having the structure of Formula (Ib):

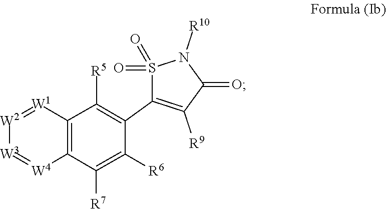

wherein at least one of W¹, W², W³, and W⁴ is N.
EMBODIMENT 14. The compound of any one of Embodiments 1-4, or a pharmaceutically acceptable salt or solvate thereof, having the structure of Formula (Ic):

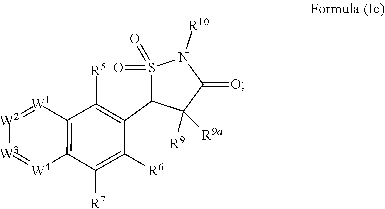

wherein at least one of W¹, W², W³, and W⁴ is N.
EMBODIMENT 15. The compound of any one of Embodiments 1-14, or a pharmaceutically acceptable salt or solvate thereof, wherein W¹ is N, W² is C(R²), W³ is C(R³), and W⁴ is C(R⁴).
EMBODIMENT 16. The compound of any one of Embodiments 1-14, or a pharmaceutically acceptable salt or solvate thereof, wherein W¹ is C(R¹), W² is N, W³ is C(R³), and W⁴ is C(R⁴).
EMBODIMENT 17. The compound of any one of Embodiments 1-14, or a pharmaceutically acceptable salt or solvate thereof, wherein W¹ is C(R¹), W² is C(R²), W³ is N, and W⁴ is C(R⁴).
EMBODIMENT 18. The compound of any one of Embodiments 1-14, or a pharmaceutically acceptable salt or solvate thereof, wherein W¹ is C(R¹), W² is C(R²), W³ is C(R³), and W⁴ is N.
EMBODIMENT 19. The compound of any one of Embodiments 1-4, or a pharmaceutically acceptable salt or solvate thereof, having the structure of Formula (Ig):

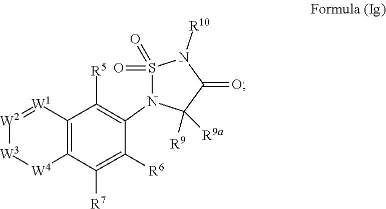

wherein at least one of W¹, W², W³, and W⁴ is N, N(R³), N(R⁴), or O.
EMBODIMENT 20. The compound of any one of Embodiments 1-4, or a pharmaceutically acceptable salt or solvate thereof, having the structure of Formula (Ih):

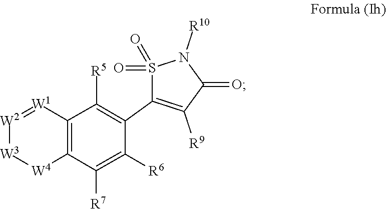

wherein at least one of W¹, W², W³, and W⁴ is N, N(R³), N(R⁴), or O.
EMBODIMENT 21. The compound of any one of Embodiments 1-4, or a pharmaceutically acceptable salt or solvate thereof, having the structure of Formula (Ii):

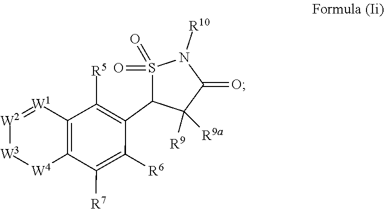

wherein at least one of W¹, W², W³, and W⁴ is N, N(R³), N(R⁴), or O.
EMBODIMENT 22. The compound of any one of Embodiments 19-21, or a pharmaceutically acceptable salt or solvate thereof, wherein W¹ is C(R¹) and W² is C(R²).
EMBODIMENT 23. The compound of any one of Embodiments 19-22, or a pharmaceutically acceptable salt or solvate thereof, wherein W³ is N(R³) and W⁴ is C(O).
EMBODIMENT 24 The compound of any one of Embodiments 19-22, or a pharmaceutically acceptable salt or solvate thereof, wherein W³ is C(O) and W⁴ is N(R⁴).
EMBODIMENT 25 The compound of any one of Embodiments 19-22, or a pharmaceutically acceptable salt or solvate thereof, wherein W³ is O and W⁴ is C(R⁴)(R^{4a}).
EMBODIMENT 26 The compound of any one of Embodiments 1-4, or a pharmaceutically acceptable salt or solvate thereof, having the structure of Formula (Ij):

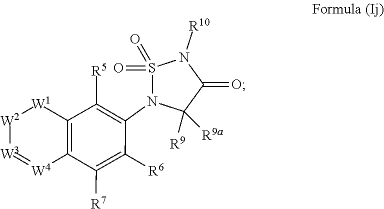

wherein at least one of W¹, W², W³, and W⁴ is N(R¹), N(R²), N, or O.
EMBODIMENT 27 The compound of any one of Embodiments 1-4, or a pharmaceutically acceptable salt or solvate thereof, having the structure of Formula (Ik):

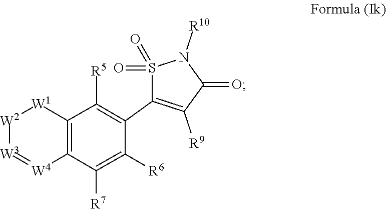

wherein at least one of W¹, W², W³, and W⁴ is N(R¹), N(R²), N, or O.
EMBODIMENT 28 The compound of any one of Embodiments 1-4, or a pharmaceutically acceptable salt or solvate thereof, having the structure of Formula (Im):

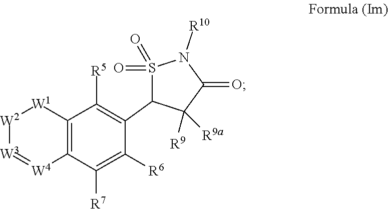

wherein at least one of W¹, W², W³, and W⁴ is N, N(R³), N(R⁴), or 0.
EMBODIMENT 29 The compound of any one of Embodiments 26-28, or a pharmaceutically acceptable salt or solvate thereof, wherein W¹ is C(R¹)(R^{1a}), W² is O, W³ is C(R³), and W⁴ is C(R⁴).
EMBODIMENT 30 The compound of any one of Embodiments 26-28, or a pharmaceutically acceptable salt or solvate thereof, wherein W¹ is CO, N(R1) or C(R¹)(R^{1a}); W² is CO, N(R²), or C(R²)(R^{2a}); W³ is N, or C(R³); and W⁴ is N, or C(R⁴).
EMBODIMENT 31 The compound of any one of Embodiments 1-4, or a pharmaceutically acceptable salt or solvate thereof, having the structure of Formula (Iv):

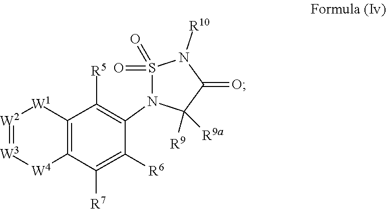

wherein at least one of W¹, W², W³, and W⁴ is N(R¹), N(R⁴), N, or O.
EMBODIMENT 3 The compound of any one of Embodiments 1-4, or a pharmaceutically acceptable salt or solvate thereof, having the structure of Formula (Iw):

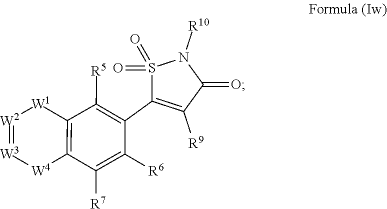

wherein at least one of W¹, W², W³, and W⁴ is N(R¹), N(R⁴), N, or O.
EMBODIMENT 33 The compound of any one of Embodiments 1-4, or a pharmaceutically acceptable salt or solvate thereof, having the structure of Formula (Ix):

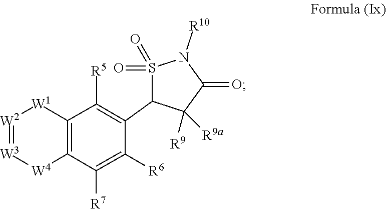

wherein at least one of W¹, W², W³, and W⁴ is N(R¹), N(R⁴), N, or O.
EMBODIMENT 34 The compound of any one of Embodiments 1-4, or a pharmaceutically acceptable salt or solvate thereof, having the structure of Formula (Io):

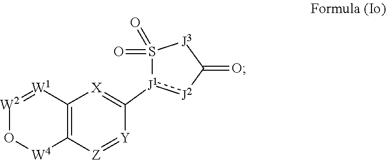

wherein W¹ is C(R¹); W² is C(R²); and W⁴ is C(R⁴)(R^{4a}), C(O), S(O), S(O)₂, S(O)(NR⁴), S(O)(R⁴), or P(O)(R⁴).
EMBODIMENT 35 The compound of Embodiment 34, or a pharmaceutically acceptable salt or solvate thereof, having the structure of Formula (Ioa):

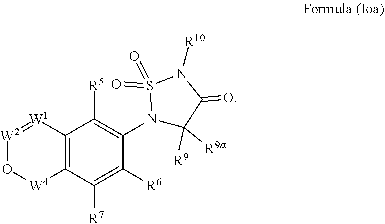

EMBODIMENT 36 The compound of Embodiment 34, or a pharmaceutically acceptable salt or solvate thereof, having the structure of Formula (Iob):

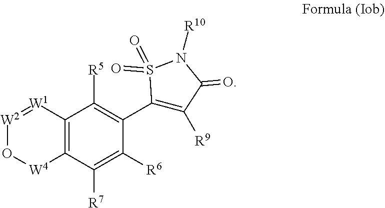

EMBODIMENT 37 The compound of Embodiment 34, or a pharmaceutically acceptable salt or solvate thereof, having the structure of Formula (Ioc):

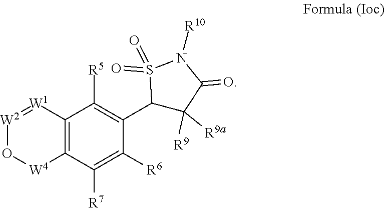

EMBODIMENT 38 The compound of any one of Embodiments 1-4, or a pharmaceutically acceptable salt or solvate thereof, having the structure of Formula (Ip):

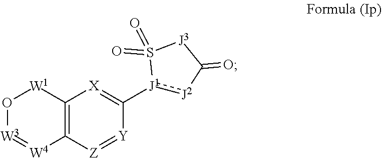

wherein: W¹ is C(R¹)(R^{1a}), C(O), S(O), S(O)₂, S(O)(NR¹), S(O)(R¹), or P(O)(R¹); W³ is C(R³); and W⁴ is C(R⁴).
EMBODIMENT 39 The compound of Embodiment 38, or a pharmaceutically acceptable salt or solvate thereof, having the structure of Formula (Ipa):

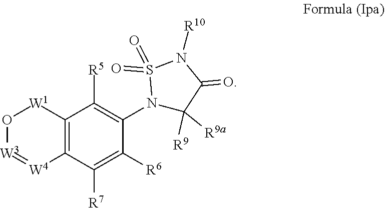

EMBODIMENT 40 The compound of Embodiment 38, or a pharmaceutically acceptable salt or solvate thereof, having the structure of Formula (Ipb):

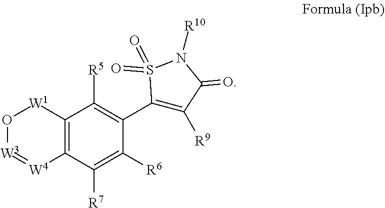

EMBODIMENT 41 The compound of Embodiment 38, or a pharmaceutically acceptable salt or solvate thereof, having the structure of Formula (Ipc):

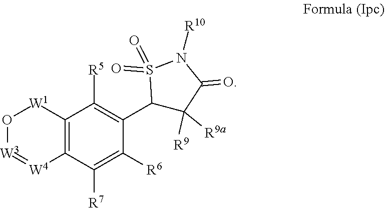

EMBODIMENT 42 The compound of any one of Embodiments 1-4, or a pharmaceutically acceptable salt or solvate thereof, having the structure of Formula (Ipd):

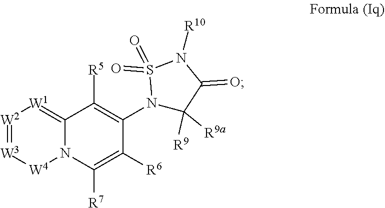

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R⁴), or P(O)(R⁴).
EMBODIMENT 43 The compound of any one of Embodiments 1-4, or a pharmaceutically acceptable salt or solvate thereof, having the structure of Formula (Iq):

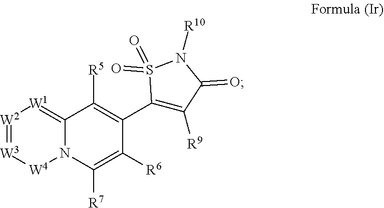

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R⁴), or P(O)(R⁴).
EMBODIMENT 44 The compound of any one of Embodiments 1-4, or a pharmaceutically acceptable salt or solvate thereof, having the structure of Formula (Ir):

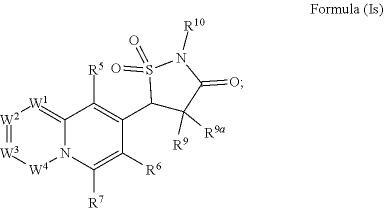

wherein W¹ is N or C(R¹); W² is N or C(R²); W³ is N or C(R³); and W⁴ is N(R⁴), C(R⁴)(R^{4a}), C(O), S(O), S(O)₂, S(O)(NR⁴), S(O)(R⁴), or P(O)(R⁴).
EMBODIMENT 45 The compound of any one of Embodiments 42-44, or a pharmaceutically acceptable salt or solvate thereof, wherein W⁴ is C(O).
EMBODIMENT 46 A compound of Formula (III), or a pharmaceutically acceptable salt or solvate thereof:

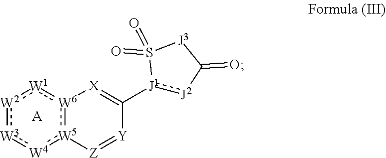

wherein:
W¹ is N, N(R¹), O, C(R), C(R¹)(R^{1a}), C(O), S(O), S(O)₂, S(O)(NR¹), S(O)(R¹), or P(O)(R¹);
W² is N, N(R²), O, C(R²), C(R²)(R²), C(O), S(O), S(O)₂, S(O)(NR²), S(O)(R²), or P(O)(R²);
W³ is N, N(R³), O, C(R³), C(R³)(R^{3a}), C(O), S(O), S(O)₂, S(O)(NR³), S(O)(R³), or P(O)(R³);
W⁴ is N, N(R⁴), O, C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein
at least one of W¹, W², W³, W⁴, W⁵, and W⁶ is N, N(R¹), N(R²), N(R³), N(R⁴), or O; and
at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁_.heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR⁶Ib) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl;
[symbol] indicates a single or double bond such that all valences are satisfied; and
provided the compound is not

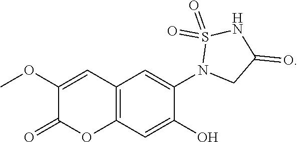 [SMILES: COc1cc2cc(N3CC(=O)NS3(=O)=O)c(O)cc2oc1=O]

EMBODIMENT 47 The compound of Embodiment 46, or a pharmaceutically acceptable salt or solvate thereof, wherein X is C(R⁵).
EMBODIMENT 48 The compound of one of Embodiments 46-47, or a pharmaceutically acceptable salt or solvate thereof, wherein Y is C(R⁶).
EMBODIMENT 49 The compound of any one of Embodiments 46-48, or a pharmaceutically acceptable salt or solvate thereof, wherein Z is C(R⁷).
EMBODIMENT 50 The compound of any one of Embodiments 46-49, or a pharmaceutically acceptable salt or solvate thereof, wherein J¹ is N and J² is C(R⁹)(R^{9a})
EMBODIMENT 51 The compound of any one of Embodiments 46-49, or a pharmaceutically acceptable salt or solvate thereof, wherein J¹ is C(R⁸) and J² is C(R⁹)(R^{9a})
EMBODIMENT 52 The compound of any one of Embodiments 46-49, or a pharmaceutically acceptable salt or solvate thereof, wherein J² is CH₂.
EMBODIMENT 53 The compound of any one of Embodiments 46-49, or a pharmaceutically acceptable salt or solvate thereof, wherein J¹ is C and J² is C(R).
EMBODIMENT 54 The compound of Embodiment 53, or a pharmaceutically acceptable salt or solvate thereof, wherein J² is C(H).
EMBODIMENT 55 The compound of any one of Embodiments 46-54, or a pharmaceutically acceptable salt or solvate thereof, wherein J³ is N(R¹⁰).
EMBODIMENT 56 The compound of any one of Embodiments 46-54, or a pharmaceutically acceptable salt or solvate thereof, wherein J³ is N(H).
EMBODIMENT 57 The compound of Embodiment 46, or a pharmaceutically acceptable salt or solvate thereof, having the structure of Formula (IIIa):

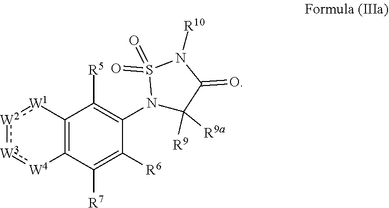

EMBODIMENT 58 The compound of Embodiment 46, or a pharmaceutically acceptable salt or solvate thereof, having the structure of Formula (IIIb):

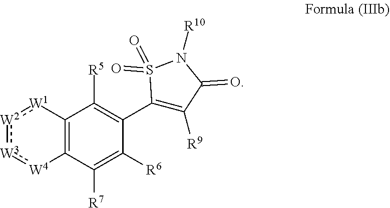

EMBODIMENT 59 The compound of Embodiment 46, or a pharmaceutically acceptable salt or solvate thereof, having the structure of Formula (IIIc):

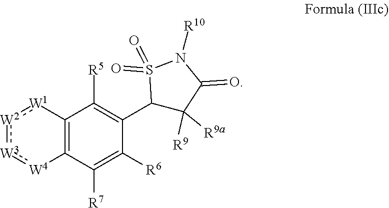

EMBODIMENT 60 The compound of any one of Embodiments 46-59, or a pharmaceutically acceptable salt or solvate thereof, wherein W¹ is C(O).
EMBODIMENT 61 The compound of Embodiment 60, or a pharmaceutically acceptable salt or solvate thereof, wherein W² is N(R²).
EMBODIMENT 62 The compound of Embodiment 61, or a pharmaceutically acceptable salt or solvate thereof, wherein W³ and W⁴ are connected by a double bond.
EMBODIMENT 63 The compound of any one of Embodiments 46-59, or a pharmaceutically acceptable salt or solvate thereof, wherein W² is C(O).
EMBODIMENT 64 The compound of Embodiment 53, or a pharmaceutically acceptable salt or solvate thereof, wherein W¹ is is N(R¹) and W³ and W⁴ are connected by a double bond.
EMBODIMENT 65 The compound of any one of Embodiments 46-59, or a pharmaceutically acceptable salt or solvate thereof, wherein W³ is C(O).
EMBODIMENT 66 The compound of Embodiment 65, or a pharmaceutically acceptable salt or solvate thereof, wherein W² is N(R²) and W¹ is C(R¹)(R^{1a})
EMBODIMENT 67 The compound of one of Embodiments 65-66, or a pharmaceutically acceptable salt or solvate thereof, wherein W⁴ is N(R⁴).
EMBODIMENT 68 The compound of one of Embodiments 65-66, or a pharmaceutically acceptable salt or solvate thereof, wherein W⁴ is C(R⁴)(R^{4a})
EMBODIMENT 69 The compound of Embodiment 65, or a pharmaceutically acceptable salt or solvate thereof, wherein W⁴ is N(R⁴) and W¹ and W² are connected by a double bond.
EMBODIMENT 70 The compound of any one of Embodiments 46-59, or a pharmaceutically acceptable salt or solvate thereof, wherein W⁴ is C(O).
EMBODIMENT 71 The compound of Embodiment 70, or a pharmaceutically acceptable salt or solvate thereof, wherein W¹ is O.
EMBODIMENT 72 The compound of one of Embodiments 70-71, or a pharmaceutically acceptable salt or solvate thereof, wherein W² is C(R²)(R^{2a}) and W³ is C(R³)(R^{3a})
EMBODIMENT 73 The compound of one of Embodiments 70-71, or a pharmaceutically acceptable salt or solvate thereof, wherein W² is C(R²) and W³ is C(R³).
EMBODIMENT 74 The compound of Embodiment 46, or a pharmaceutically acceptable salt or solvate thereof, having the structure of Formula (IIId):

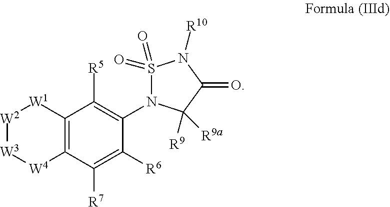

EMBODIMENT 75 The compound of Embodiment 46, or a pharmaceutically acceptable salt or solvate thereof, having the structure of Formula (IIIe):

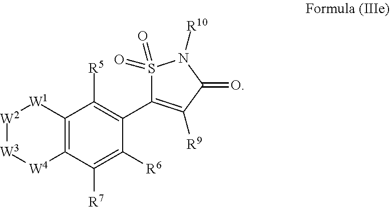

EMBODIMENT 76 The compound of Embodiment 46, or a pharmaceutically acceptable salt or solvate thereof, having the structure of Formula (IIIf):

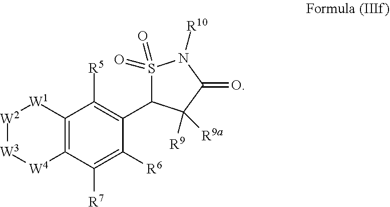

EMBODIMENT 77 The compound of Embodiment 46, or a pharmaceutically acceptable salt or solvate thereof, having the structure of Formula (IIIg):

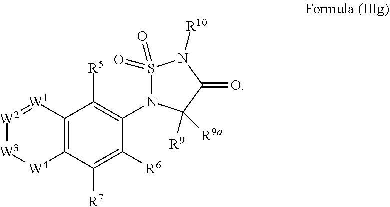

EMBODIMENT 78 The compound of Embodiment 46, or a pharmaceutically acceptable salt or solvate thereof, having the structure of Formula (IIIh):

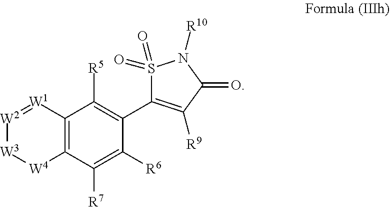

EMBODIMENT 79 The compound of Embodiment 46, or a pharmaceutically acceptable salt or solvate thereof, having the structure of Formula (IIIi):

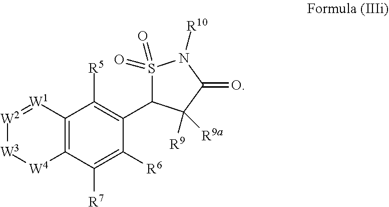

EMBODIMENT 80 The compound of Embodiment 46, or a pharmaceutically acceptable salt or solvate thereof, having the structure of Formula (IIIj):

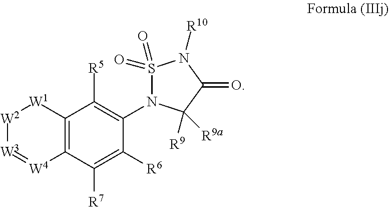

EMBODIMENT 81 The compound of Embodiment 46, or a pharmaceutically acceptable salt or solvate thereof, having the structure of Formula (IIIk):

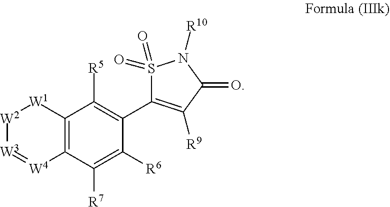

EMBODIMENT 82 The compound of Embodiment 46, or a pharmaceutically acceptable salt or solvate thereof, having the structure of Formula (IIII):

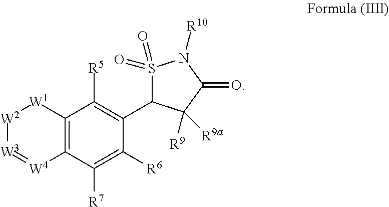

EMBODIMENT 83 The compound of Embodiment 46, or a pharmaceutically acceptable salt or solvate thereof, having the structure of Formula (IIIm):

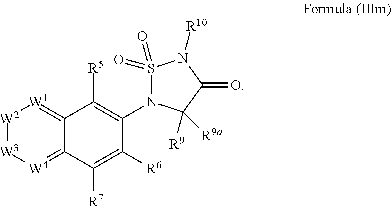

EMBODIMENT 84 The compound of Embodiment 46, or a pharmaceutically acceptable salt or solvate thereof, having the structure of Formula (IIIn):

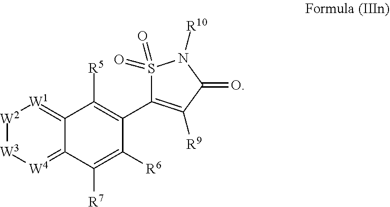

EMBODIMENT 85 The compound of Embodiment 46, or a pharmaceutically acceptable salt or solvate thereof, having the structure of Formula (IIIo):

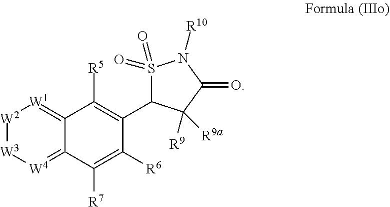

EMBODIMENT 86 The compound of Embodiment 46, or a pharmaceutically acceptable salt or solvate thereof, having the structure of Formula (IIIp):

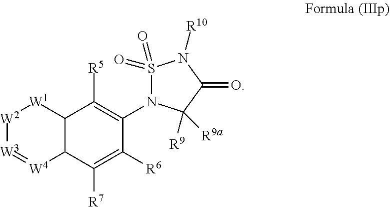

EMBODIMENT 87 The compound of Embodiment 46, or a pharmaceutically acceptable salt or solvate thereof, having the structure of Formula (IIIq)

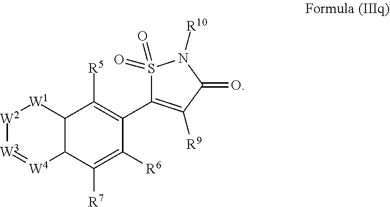

EMBODIMENT 88 The compound of Embodiment 46, or a pharmaceutically acceptable salt or solvate thereof, having the structure of Formula (IIIr):

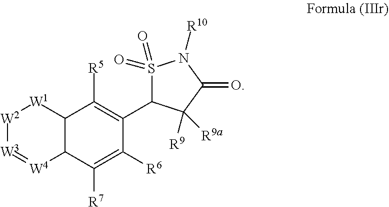

EMBODIMENT 89 The compound of Embodiment 46, or a pharmaceutically acceptable salt or solvate thereof, having the structure of Formula (IIIs):

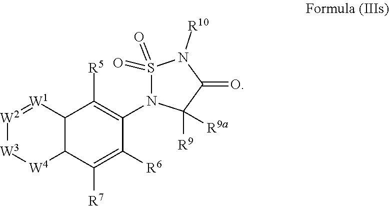

EMBODIMENT 90 The compound of Embodiment 46, or a pharmaceutically acceptable salt or solvate thereof, having the structure of Formula (IIIt):

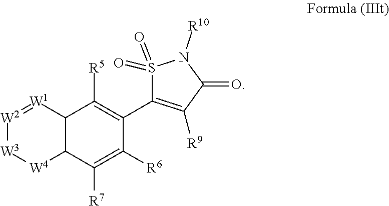

EMBODIMENT 91 The compound of Embodiment 46, or a pharmaceutically acceptable salt or solvate thereof, having the structure of Formula (IIIu):

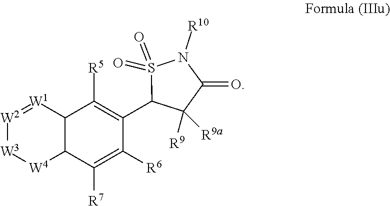

EMBODIMENT 92 A compound of Formula (II), or a pharmaceutically acceptable salt or solvate thereof:

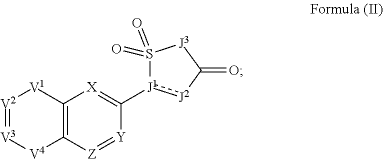

wherein:
V¹ is a bond or S;
V⁴ is a bond or S, wherein when V¹ is a bond then V⁴ is S and when V¹ is S, then V⁴ is a bond;
V² is N or C(R²);
V³ is N or C(R³);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_9 S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR⁶b) and —P(O)(OR^{16a})(OR^{16b}); wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl; and
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl.
EMBODIMENT 93 The compound of Embodiment 92, or a pharmaceutically acceptable salt or solvate thereof, wherein X is C(R⁵).
EMBODIMENT 94 The compound of one of Embodiments 92-93, or a pharmaceutically acceptable salt or solvate thereof, wherein Y is C(R⁶).
EMBODIMENT 95 The compound of any one of Embodiments 92-94, or a pharmaceutically acceptable salt or solvate thereof, wherein Z is C(R⁷).
EMBODIMENT 96 The compound of any one of Embodiments 92-95, or a pharmaceutically acceptable salt or solvate thereof, wherein J¹ is N and J² is C(R⁹)(R^{9a})
EMBODIMENT 97 The compound of any one of Embodiments 92-95, or a pharmaceutically acceptable salt or solvate thereof, wherein J¹ is C(R⁸) and J² is C(R⁹)(R^{9a})
EMBODIMENT 98 The compound of any one of Embodiments 92-97, or a pharmaceutically acceptable salt or solvate thereof, wherein J² is CH₂.
EMBODIMENT 99 The compound of any one of Embodiments 92-95, or a pharmaceutically acceptable salt or solvate thereof, wherein J¹ is C and J² is C(R⁹).
EMBODIMENT 100 The compound of Embodiment 99, or a pharmaceutically acceptable salt or solvate thereof, wherein J² is C(H).
EMBODIMENT 101 The compound of any one of Embodiments 92-100, or a pharmaceutically acceptable salt or solvate thereof, wherein J³ is N(R¹⁰).
EMBODIMENT 102 The compound of any one of Embodiments 92-100, or a pharmaceutically acceptable salt or solvate thereof, wherein J³ is N(H).
EMBODIMENT 103 The compound of any one of Embodiments 92-102, or a pharmaceutically acceptable salt or solvate thereof, wherein V¹ is a bond and V⁴ is S.
EMBODIMENT 104 The compound of any one of Embodiments 92-102, or a pharmaceutically acceptable salt or solvate thereof, wherein V¹ is S and V⁴ is a bond.
EMBODIMENT 105 The compound of Embodiment 92, or a pharmaceutically acceptable salt or solvate thereof, having the structure of Formula (IIa):

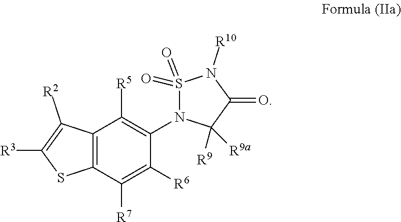

EMBODIMENT 106 The compound of Embodiment 92, or a pharmaceutically acceptable salt or solvate thereof, having the structure of Formula (IIb):

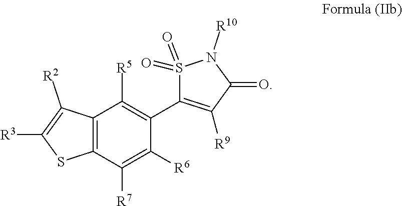

EMBODIMENT 107 The compound of Embodiment 92, or a pharmaceutically acceptable salt or solvate thereof, having the structure of Formula (IIc):

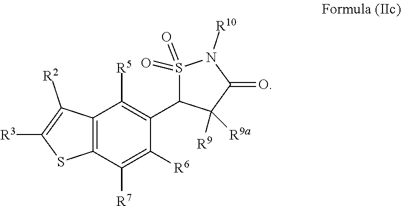

EMBODIMENT 108 The compound of Embodiment 92, or a pharmaceutically acceptable salt or solvate thereof, having the structure of Formula (IId):

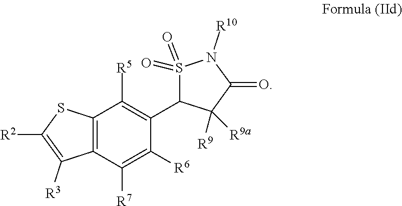

EMBODIMENT 109 The compound of Embodiment 92, or a pharmaceutically acceptable salt or solvate thereof, having the structure of Formula (IIe):

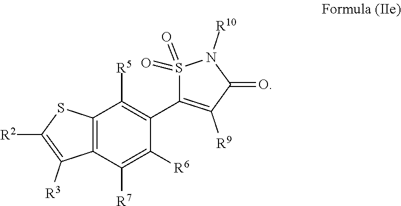

EMBODIMENT 110 The compound of Embodiment 92, or a pharmaceutically acceptable salt or solvate thereof, having the structure of Formula (IIf):

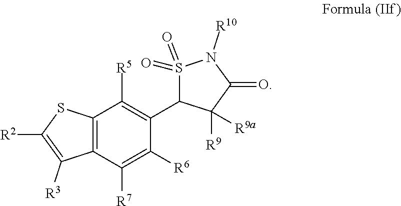

EMBODIMENT 111 The compound of any one of Embodiments 1-110, or a pharmaceutically acceptable salt or solvate thereof, wherein R⁵ is selected from hydrogen, halogen, and C₁₋₆alkyl optionally substituted with one, two, or three R^{20e}
EMBODIMENT 112 The compound of any one of Embodiments 1-110, or a pharmaceutically acceptable salt or solvate thereof, wherein R⁵ is selected from hydrogen and halogen.
EMBODIMENT 113 The compound of any one of Embodiments 1-112, or a pharmaceutically acceptable salt or solvate thereof, wherein R⁶ is selected from hydrogen, halogen, —OR¹², and C₁₋₆alkyl optionally substituted with one, two, or three R^{20f}.
EMBODIMENT 114 The compound of any one of Embodiments 1-112, or a pharmaceutically acceptable salt or solvate thereof, wherein R⁶ is —OH.
EMBODIMENT 115 The compound of any one of Embodiments 1-114, or a pharmaceutically acceptable salt or solvate thereof, wherein R⁷ is selected from hydrogen, halogen, and C₁₋₆alkyl optionally substituted with one, two, or three R^{20g}
EMBODIMENT 116 The compound of any one of Embodiments 1-114, or a pharmaceutically acceptable salt or solvate thereof, wherein R⁷ is hydrogen.
EMBODIMENT 117 The compound of any one of Embodiments 1-116, or a pharmaceutically acceptable salt or solvate thereof, wherein R⁹ is hydrogen.
EMBODIMENT 118 The compound of any one of Embodiments 1-117, or a pharmaceutically acceptable salt or solvate thereof, wherein R^{9a} is hydrogen.
EMBODIMENT 119 The compound of any one of Embodiments 1-118, or a pharmaceutically acceptable salt or solvate thereof, wherein R¹⁰ is hydrogen.
EMBODIMENT 120 The compound of any one of Embodiments 1-119, or a pharmaceutically acceptable salt or solvate thereof, wherein R² is selected from hydrogen, halogen, C₁₋₆alkyl, C₂₋₉heterocycloalkyl, C₁₋₉heteroaryl, —OR¹², —SR¹², —N(R¹²)(R¹³), —C(O)R¹⁵, —C(O)N(R¹²)(R¹³), —S(O)₂R¹⁵, and —S(O)₂N(R¹²)(R¹³)—, wherein C₁₋₆alkyl, C₂₋₉heterocycloalkyl, and C₁₋₉ heteroaryl are optionally substituted with one, two, or three R^{20b}
EMBODIMENT 121 The compound of any one of Embodiments 1-119, or a pharmaceutically acceptable salt or solvate thereof, wherein R² is selected from C₁₋₆alkyl, C₂₋₉heterocycloalkyl, C₁₋₉heteroaryl, —OR¹², —N(R¹²)(R¹³), —C(O)R¹⁵, —C(O)N(R¹²)(R¹³), wherein C₁₋₆alkyl, C₂₋₉heterocycloalkyl, and C₁₋₉heteroaryl are optionally substituted with one, two, or three R^{20b}
EMBODIMENT 122 The compound of any one of Embodiments 1-119, or a pharmaceutically acceptable salt or solvate thereof, wherein R² is selected from —OR¹², —N(R¹²)(R¹³), —C(O)R¹⁵, —C(O)N(R¹²)(R¹³);
R¹³ is hydrogen;
R¹² and R¹⁵ are independently

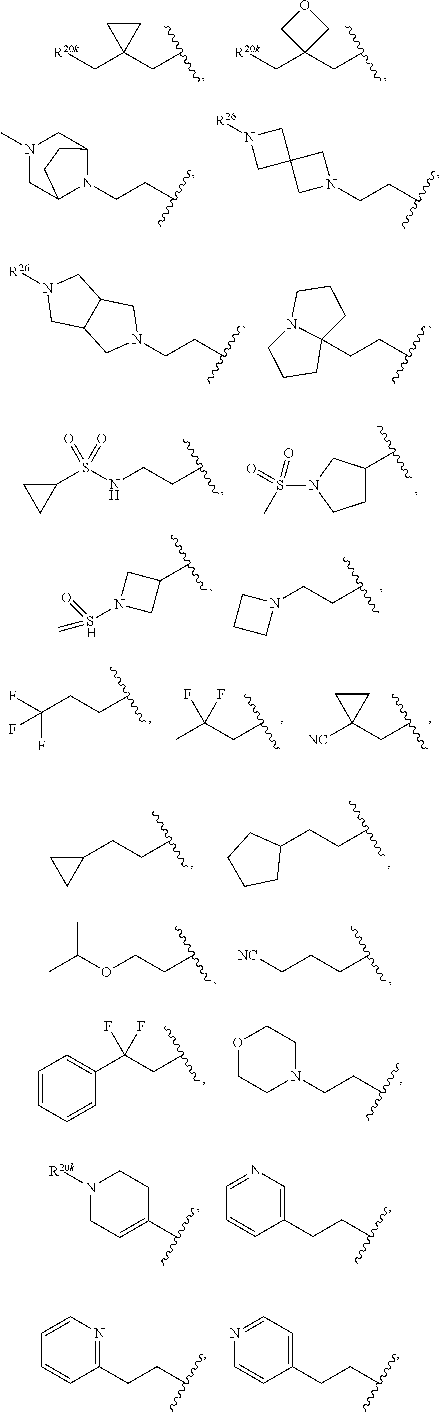

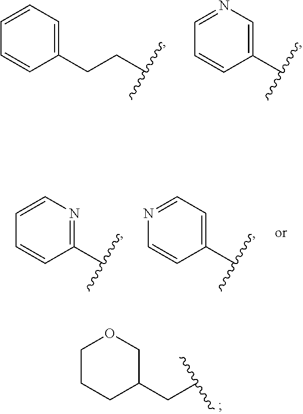 [SMILES: *CCc1ccccc1 ; *c1cccnc1 ; *c1ccccn1 ; *c1ccncc1 ; *CC1CCCOC1]

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.
EMBODIMENT 123 The compound of any one of Embodiments 1-119, or a pharmaceutically acceptable salt or solvate thereof, wherein R² is selected from —OR¹², —N(R¹²)(R¹³), —C(O)R¹⁵, —C(O)N(R¹²)(R¹³);
R¹³ is hydrogen;
R¹² and R¹⁵ are independently

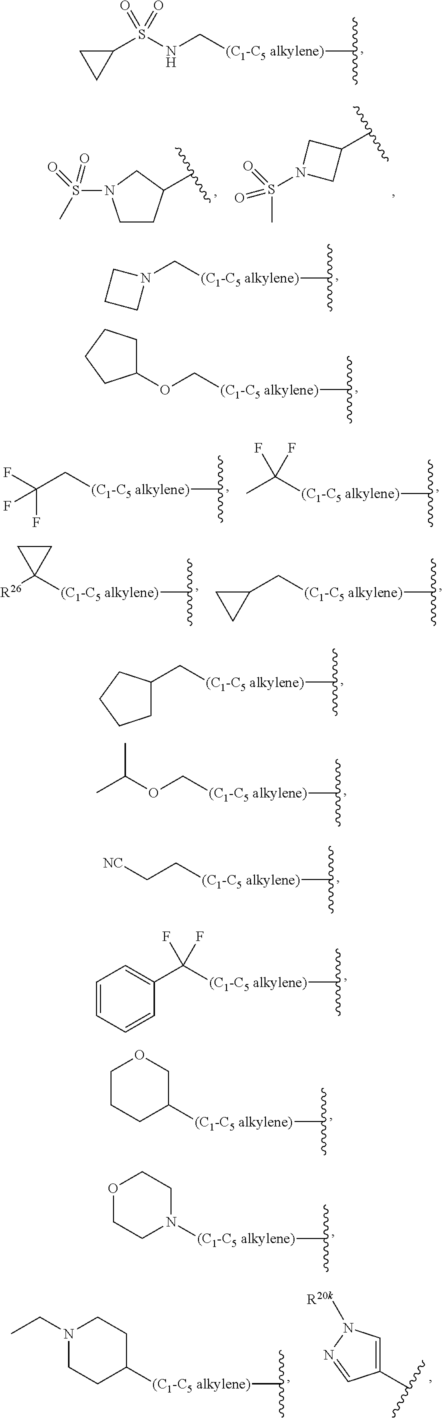

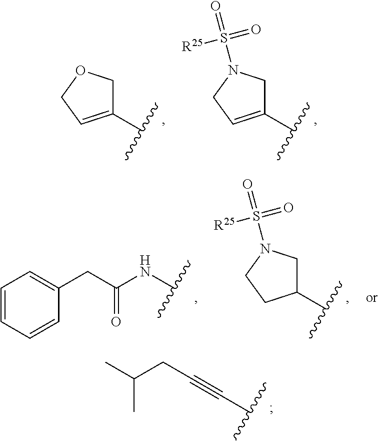

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.
EMBODIMENT 124 The compound of any one of Embodiments 1-119, or a pharmaceutically acceptable salt or solvate thereof, wherein
R² is selected from

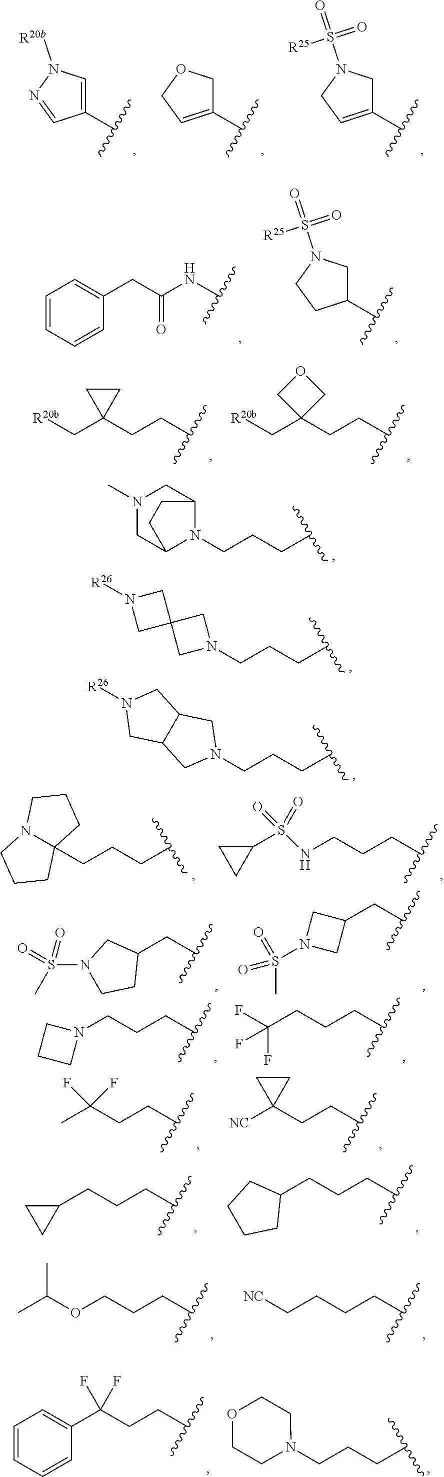

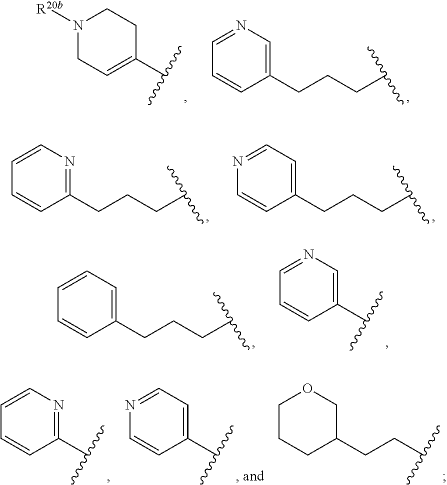

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. n
EMBODIMENT 125 The compound of any one of Embodiments 1-119, or a pharmaceutically acceptable salt or solvate thereof, wherein
R² is independently

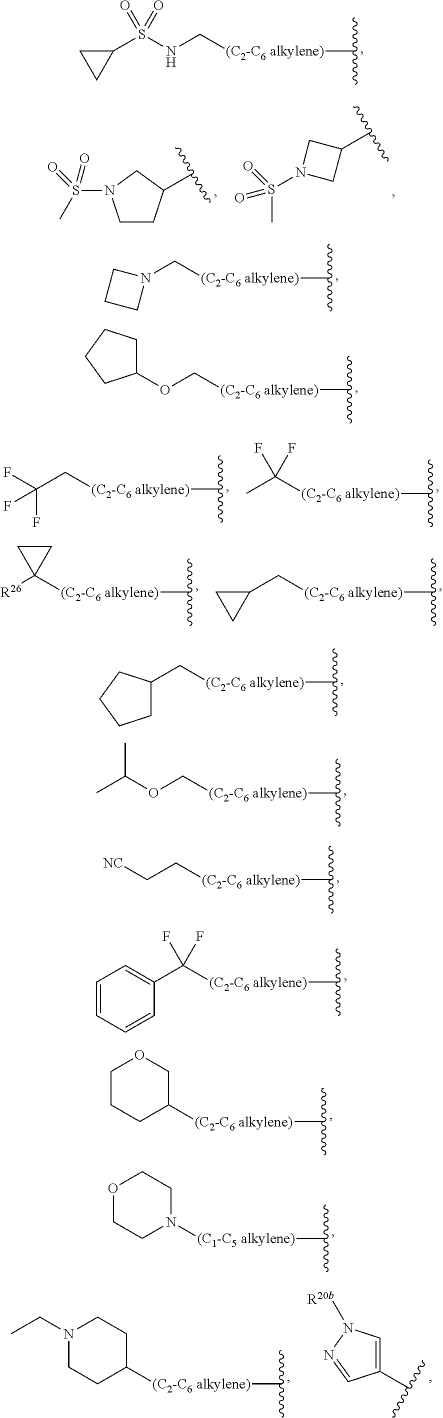

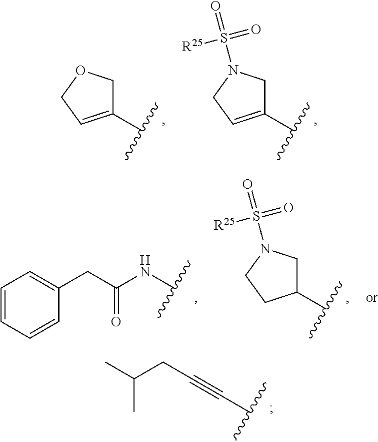

and
R²⁶ is independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.
EMBODIMENT 126 The compound of any one of Embodiments 1-125, or a pharmaceutically acceptable salt or solvate thereof, wherein R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R²¹.
EMBODIMENT 127 The compound of any one of Embodiments 1-125, or a pharmaceutically acceptable salt or solvate thereof, wherein R³ is selected from hydrogen, halogen, —CN, C₁₋₆alkyl, C₃₋₆cycloalkyl, and —OR¹², wherein C₁₋₆alkyl and C₃₋₆cycloalkyl are optionally substituted with one, two, or three R^{20c}
EMBODIMENT 128 A compound, or a pharmaceutically acceptable salt or solvate thereof, selected from:

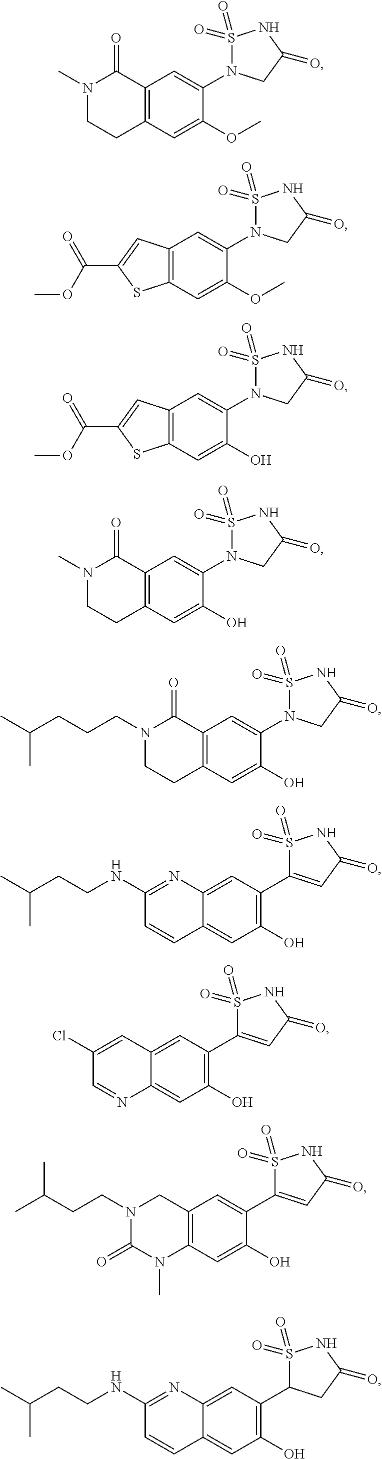

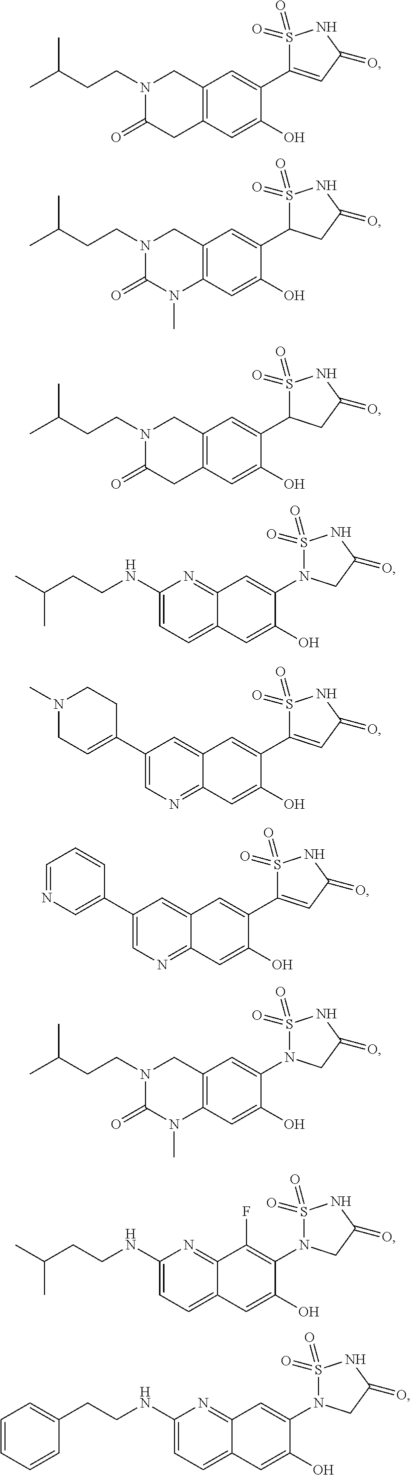

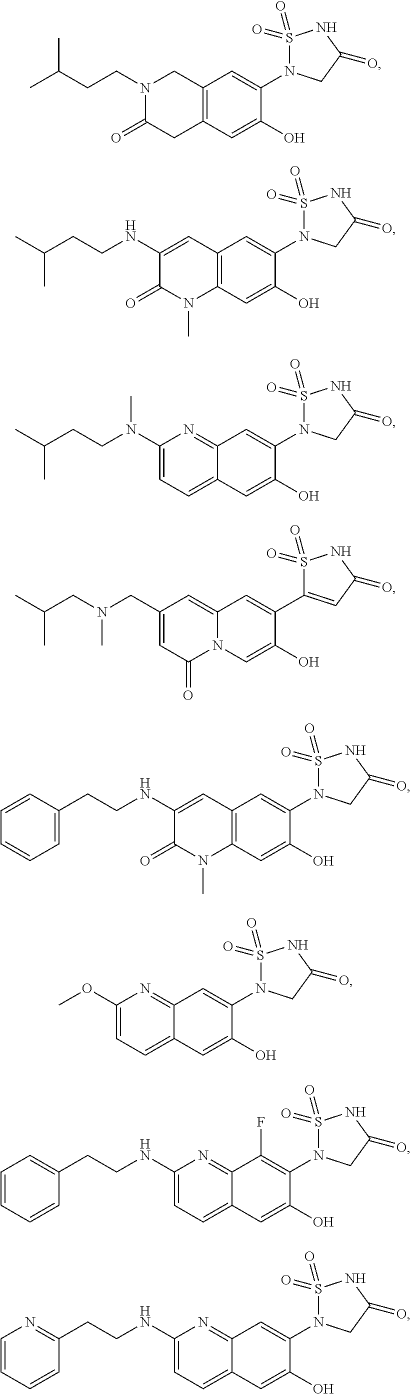

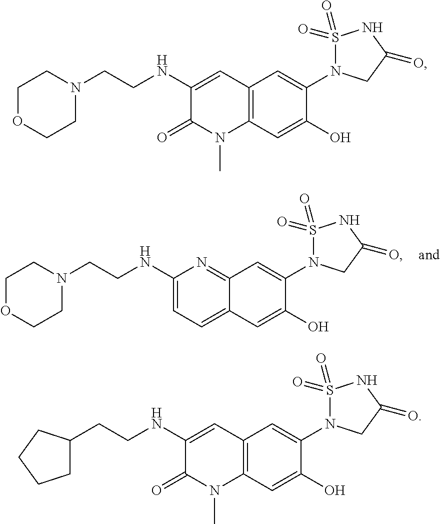

EMBODIMENT 129 A pharmaceutical composition comprising a compound of any one of Embodiments 1-128, or a pharmaceutically acceptable salt or solvate thereof, and a pharmaceutically acceptable excipient.
EMBODIMENT 130 A method of treating cancer in a subject in need thereof, comprising administering to the subject a therapeutically effective amount of a compound of any one of Embodiments 1-128, or a pharmaceutically acceptable salt or solvate thereof.
EMBODIMENT 131 A method of potentiating immunity of a cell, comprising: contacting the cell with a compound of any one of Embodiments 1-128, thereby potentiating immunity of the cell,
wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.
EMBODIMENT 132 A method of potentiating immunity of a cell, comprising:
(a) contacting the cell with a compound of any one of Embodiments 1-128; and
(b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell.
EMBODIMENT 133 The method of Embodiment 132, wherein (a) is performed prior to, concurrent with, or subsequent to (b).
EMBODIMENT 134 The method of any one of Embodiments 131-133, wherein the cell retains expression or activity of PTPN2 prior to (a).
EMBODIMENT 135 The method of any one Embodiments 131-134, wherein the cell is a lymphoid cell.
EMBODIMENT 136 The method of any one of Embodiments 131-135, further comprising administering the cell to a subject in need thereof.
EMBODIMENT 137 The method of Embodiment 136, further comprising administering the compound of any one of Embodiments 1-128 to the subject prior to, concurrent with, or subsequent to the administering the cell.
EMBODIMENT 138 The method of Embodiment 137, wherein, prior to the administering the compound of any one of Embodiments 1-128, a cell of the subject exhibits expression or activity of PTPN2.
EMBODIMENT 139 A method of potentiating immunity of a subject in need thereof, comprising:
administering a lymphoid cell to the subject, thereby potentiating immunity of the subject,
wherein expression or activity of PTPN2 in the lymphoid cell is transiently downregulated.
EMBODIMENT 140 The method of Embodiment 139, further comprising transiently downregulating the expression or activity of PTPN2 in the lymphoid cell.
EMBODIMENT 141 The method of Embodiment 140, wherein, prior to the transiently downregulating, the lymphoid cell exhibits expression or activity of PTPN2.
EMBODIMENT 142 The method of Embodiment 140, wherein the transiently downregulating is performed once.
EMBODIMENT 143 The method of Embodiment 140, wherein the transiently downregulating is performed intermittently for two or more times.
EMBODIMENT 144 The method of any one of Embodiments 140-143, wherein the transiently downregulating comprises introducing a compound of any one of Embodiments 1-128 to the cell.
EMBODIMENT 145 The method of Embodiment 144, wherein a first intermittent dosing regimen of the compound and a second intermittent dosing regimen of the compound is the same.
EMBODIMENT 146 The method of Embodiment 144, wherein a first intermittent dosing regimen of the compound and a second intermittent dosing regimen of the compound are different.
EMBODIMENT 147 The method of any one of Embodiments 139-146, wherein the lymphoid cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.
EMBODIMENT 148 The method of any one of Embodiments 139-147, further comprising administering a compound of any one of Embodiments 1-128 to the subject prior to, concurrent with, or subsequent to the administering the lymphoid cell.
EMBODIMENT 149 The method of Embodiment 148, wherein, prior to the administering the compound, a cell of the subject exhibits expression or activity of PTPN2.
EMBODIMENT 150 A method of potentiating immunity of a subject in need thereof, comprising:
(a) selecting the subject that exhibits expression or activity of PTPN2; and
(b) downregulating expression or activity of PTPN2 in a cell of the subject, thereby potentiating immunity of the subject.
EMBODIMENT 151 The method of Embodiment 150, wherein the step (b) is performed in vivo.
EMBODIMENT 152 The method of Embodiment 150, wherein the step (b) is performed ex vivo.
EMBODIMENT 153 The method of Embodiment 150, further comprising administering the cell to the subject prior to, concurrent with, or subsequent to the downregulating.
EMBODIMENT 154 The method of any one of Embodiments 150-153, wherein the downregulating comprises introducing a compound of any one of Embodiments 1-128 to the cell.
EMBODIMENT 155 The method of any one of Embodiments 150-154, wherein the downregulating comprises transiently downregulating the expression or activity of PTPN2.
EMBODIMENT 156 The method of Embodiment 155, wherein the transiently downregulating is performed once.
EMBODIMENT 157 The method of Embodiment 155, wherein the transiently downregulating is performed intermittently for two or more times.
EMBODIMENT 158 The method of Embodiment 157, wherein a first intermittent dosing regimen of the compound and a second intermittent dosing regimen of the compound is the same.
EMBODIMENT 159 The method of Embodiment 157, wherein a first intermittent dosing regimen of the compound and a second intermittent dosing regimen of the compound are different.
EMBODIMENT 160 The method of any one of Embodiments 150-159, wherein the cell of the subject is a lymphoid cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.
EMBODIMENT 161 The method of any one of Embodiments 150-160, wherein the cell of the subject does not exhibit a mutation of (i) a first gene encoding PTPN2 or (ii) a second gene operatively linked to PTPN2, wherein the mutation inhibits the expression and/or activity of PTPN2.
EMBODIMENT 162 The method of Embodiment 161, wherein the selecting comprises performing a nucleic acid assay using at least a portion of a genome or transcriptome of the cell of the subject to detect the mutation.
EMBODIMENT 163 The method of Embodiment 161, wherein the selecting comprises performing a protein assay to detect a functionally active PTPN2 or a functionally inactive PTPN2.
EMBODIMENT 164 A method of potentiating immunity of a subject in need thereof, comprising:
administering a lymphoid cell to the subject; and
administering a compound of any one of Embodiments 1-128 to the subject, thereby potentiating immunity of the subject.
EMBODIMENT 165 The method of Embodiment 164, wherein the administering the compound is performed prior to, concurrent with, or subsequent to the administering the lymphoid cell.
EMBODIMENT 166 The method of Embodiment 165, wherein the administering the compound is performed separately from the administering the lymphoid cell.
EMBODIMENT 167 The method of any one of Embodiments 164-166, wherein, prior to the administering the compound, a cell of the subject exhibits expression or activity of PTPN2.
EMBODIMENT 168 A method of potentiating anti-tumor or anti-cancer immunity of a subject in need thereof, comprising:
(a) contacting a lymphoid cell of the subject with a compound of any one of Embodiments 1-128, thereby potentiating the anti-tumor or anti-cancer immunity of the subject.
EMBODIMENT 169 A method of treating tumor or cancer of a subject in need thereof, comprising:
(a) contacting a lymphoid cell of the subject with a compound of any one of Embodiments 1-128, thereby treating the tumor or cancer of the subject.
EMBODIMENT 170 The method of any one of Embodiments 168-169, wherein the contacting is performed in vivo.
EMBODIMENT 171 The method of any one of Embodiments 168-169, wherein the contacting is performed ex vivo, and subsequently followed by introducing the lymphoid cell to the subject.
EMBODIMENT 172 The method of any one of Embodiments 168-169, further comprising administering the lymphoid cell to the subject prior to, concurrent with, or subsequent to the contacting.
EMBODIMENT 173 The method of any one of Embodiments 168-169, further comprising (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.
EMBODIMENT 174 The method of Embodiment 173, wherein (a) is performed prior to, concurrent with, or subsequent to (b).
EMBODIMENT 175 A method of potentiating anti-tumor or anti-cancer immunity of a subject in need thereof, comprising:
(a) downregulating expression or activity of PTPN2 in a lymphoid cell of the subject, thereby potentiating the anti-tumor or anti-cancer immunity of the subject.
EMBODIMENT 176 A method of treating tumor or cancer of a subject in need thereof, comprising:
(a) downregulating expression or activity of PTPN2 in a lymphoid cell of the subject, thereby treating the tumor or cancer of the subject.
EMBODIMENT 177 The method of any one of Embodiments 175-176, wherein the downregulating is performed is performed in vivo.
EMBODIMENT 178 The method of any one of Embodiments 175-176, wherein the downregulating is performed is performed ex vivo, and subsequently followed by introducing the lymphoid cell to the subject.
EMBODIMENT 179 The method of any one of Embodiments 175-176, further comprising administering the lymphoid cell to the subject prior to, concurrent with, or subsequent to the downregulating.
EMBODIMENT 180 The method of any one of Embodiments 175-179, wherein the downregulating comprises introducing a compound of any one of Embodiments 1-128 to the lymphoid cell.
EMBODIMENT 181 The method of any one of Embodiments 175-180, wherein the downregulating comprises transiently downregulating the expression or activity of PTPN2.
EMBODIMENT 182 The method of Embodiment 181, wherein the transiently downregulating is performed once.
EMBODIMENT 183 The method of Embodiment 181, wherein the transiently downregulating is performed intermittently for two or more times.
EMBODIMENT 184 The method of Embodiment 183, wherein a first intermittent dosing regimen of the compound and a second intermittent dosing regimen of the compound is the same.
EMBODIMENT 185 The method of Embodiment 183, wherein a first intermittent dosing regimen of the compound and a second intermittent dosing regimen of the compound are different.
EMBODIMENT 186 The method of any one of Embodiments 175-185, further comprising (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.
EMBODIMENT 187 The method of Embodiment 186, wherein (a) is performed prior to, concurrent with, or subsequent to (b).
EMBODIMENT 188 A method of increasing efficacy or reducing side effect of a cell therapy for a subject in need thereof, comprising:
   (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and
(b) administering a compound of any one of Embodiments 1-128 to the subject prior to, concurrent with, or subsequent to (a).
EMBODIMENT 189 The method of Embodiment 188, wherein the cell retains expression or activity of PTPN2 prior to (b).
EMBODIMENT 190 The method of Embodiment 188, wherein the cell is a lymphoid cell.
EMBODIMENT 191 The method of Embodiment 188, wherein, prior to the administering the compound, a cell of the subject exhibits expression or activity of PTPN2.
EMBODIMENT 192 A method of increasing efficacy or reducing side effect of a cell therapy for a subject in need thereof, comprising:
(a) administering to the subject a sub-therapeutic amount of a cell comprising a chimeric antigen receptor (CAR) sequence encoding a CAR,
(b) administering a compound of any one of Embodiments 1-128 to the subject prior to, concurrent with, or subsequent to (a).
EMBODIMENT 193 The method of Embodiment 192, wherein the cell retains expression or activity of PTPN2 prior to (b).
EMBODIMENT 194 The method of Embodiment 192, wherein the cell is a lymphoid cell.
EMBODIMENT 195 The method of Embodiment 192, wherein, prior to the administering the compound, a cell of the subject exhibits expression or activity of PTPN2.
EMBODIMENT 196 The method of any one of Embodiments 131-195, wherein the immunity comprises anti-tumor, anti-cancer activity, anti-viral infection activity, and/or anti-bacterial infection activity.
EMBODIMENT 197 The method of any one of Embodiments 131-196, wherein the compound of any one of Embodiments 1-128 reduces PTPN2 signaling in a cell of the subject.
EMBODIMENT 198 The method of any one of Embodiments 131-197, wherein the compound of any one of Embodiments 1-128 does not regulate site-specific recombination of a gene encoding PTPN2.
EMBODIMENT 199 The method of Embodiment 198, wherein the compound does not affect editing of (i) the gene encoding PTPN2 or (ii) an additional gene operatively linked to PTPN2.
EMBODIMENT 200 The method of Embodiment 198, wherein the compound is configured to bind PTPN2.
EMBODIMENT 201 The method of Embodiment 200, wherein the compound exhibits binding specificity to PTPN2 in comparison to other tyrosine phosphatases.
EMBODIMENT 202 The method of Embodiment 200, wherein the compound exhibits IC₅₀ of less than or equal to 5 μM for PTPN2.
EMBODIMENT 203 The method of any one Embodiments 131-202, further comprising monitoring, concurrent with or subsequent to the administration of the compound of any one of Embodiments 1-128 and/or the lymphoid cell, one or more health parameters of the subject selected from the group consisting of: temperature, wheezing, sweating, fatigue, weight, insomnia, diarrhea, infections, and mental disorders.
EMBODIMENT 204 The method of any one Embodiments 131-203, further comprising detecting, concurrent with or subsequent to the administration of the compound of any one of Embodiments 1-128 of the lymphoid cell, one or more inflammatory biomarkers selected from the group consisting of: antibodies, cytokines, radicals, and coagulation factors.
EMBODIMENT 205 The method of Embodiment 204, wherein the cytokines comprise IL-1, IL-6, TNF-α, IL-10, or IL-1RA.
EMBODIMENT 206 The method of any one Embodiments 131-205, wherein the cell of the subject comprises a diseased cell.
EMBODIMENT 207 The method of Embodiment 206, wherein the diseased cell is a tumor cell or a cancer cell.
EMBODIMENT 208 The method of any one Embodiments 131-207, wherein the cell of the subject comprises a lymphoid cell.
EMBODIMENT 209 The method of any one Embodiments 131-208, wherein the lymphoid cell is selected from the group consisting of: T cell, B cell, NK cell, KHYG cell, T helper cell, regulatory T cell, memory T cell, tumor infiltration T cell (TIL), antigen presenting cell, and dendritic cell.
EMBODIMENT 210 The method of any one Embodiments 131-209, wherein the lymphoid cell is selected from the group consisting of: a CD4+ T cell, a CD8+ T cell, and a CD4+ and CD8+ T cell.
EMBODIMENT 211 The method of any one of Embodiments 131-210, wherein the subject suffers from a cancer selected from cancer of bladder, bone, brain, breast, cervical, colon, lung, esophagus, head and neck, ovary, prostate, uterus, stomach, skin, and renal tissue.
EMBODIMENT 212 The method of any one of Embodiments 131-212, wherein (1) the contacting the cell with a compound of any one of Embodiments 1-128, (2) the administering the lymphoid cell to the subject, (3) the downregulating the expression or activity of PTPN2 in the cell of the subject, (4) the administering the compound of any one of Embodiments 1-128 to the subject, (5) the contacting the lymphoid cell of the subject with the compound of any one of Embodiments 1-128, and/or (6) the downregulating the expression or activity of PTPN2 in the lymphoid cell of the subject is performed prior to, concurrent with, or subsequent to an administration of another agent (second agent) or therapy to the subject.
EMBODIMENT 213 The method of Embodiment 212, wherein the second agent is selected from the group consisting of a chemotherapeutic agent, a radioactive agent, a small molecule agent targeting a tumor marker, an antigen-binding agent specifically binding to a tumor marker, and an immune modulator.
EMBODIMENT 214 The method of Embodiment 212, wherein the second agent is a checkpoint inhibitor.
EMBODIMENT 215 The method of Embodiment 212, wherein the second agent is an inhibitor of PD1, PD-L1, LAG3, CTLA4, CD160, BTLA, LAIR1, TIM3, 2B4, CD93, OX40, Siglec-15, and TIGIT.
EMBODIMENT 216 The method of Embodiment 212, wherein the second agent is an inhibitor of IDO or mTOR
EMBODIMENT 217 The method of Embodiment 212, wherein the therapy is a cell therapy comprising stem cells or lymphoid cells.
EMBODIMENT 218 The method of any one of Embodiments 131-217, wherein the TFP comprises a TCR subunit that comprises (1) a TCR extracellular domain capable of specific binding to an antigen, and (2) an intracellular signaling domain, wherein the TFP forms a TCR complex.
EMBODIMENT 219 The method of Embodiment 218, wherein the TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together.
EMBODIMENT 220 The method of Embodiment 218, wherein the TCR intracellular domain comprising a stimulatory domain from an intracellular signaling domain of epsilon chain, delta chain, and/or a gamma chain of cluster of differentiation 3 (CD3).
EMBODIMENT 221 The method of Embodiment 218, wherein the TCR intracellular domain comprising a stimulatory domain from an intracellular signaling domain of TCR alpha, or from an intracellular signaling domain of TCR beta.
EMBODIMENT 222 The method of any one of Embodiments 131-221, wherein the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154.
EMBODIMENT 223 The method of any one of Embodiments 131-222, wherein the TFP comprises a costimulatory domain.
EMBODIMENT 224 The method of Embodiment 223, wherein the costimulatory domain of the TFP is selected from the group consisting of: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
EMBODIMENT 225 The method of any one of Embodiments 131-224, wherein the CAR comprises an antigen-binding domain and an intracellular signaling domain.
EMBODIMENT 226 The method of Embodiment 225, wherein the intracellular signaling domain of the CAR comprises a primary signaling domain and/or a costimulatory signaling domain, wherein the primary signaling domain comprises a functional signaling domain of a protein chosen from CD3 zeta, CD3 gamma, CD3 delta, CD3 epsilon, common FcR gamma (FCERIG), FcR beta (Fc Epsilon Rib), CD79a, CD79b, Fcgamma Rlla, DAP10, or DAP12.
EMBODIMENT 227 The method of Embodiment 225, wherein the intracellular signaling domain of the CAR comprises a costimulatory signaling domain that comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
EMBODIMENT 228 The method of Embodiment 225, wherein the intracellular signaling domain of the CAR comprises a primary signaling domain and/or a costimulatory signaling domain, wherein the primary signaling domain and/or the costimulatory signaling domain is minimally required for activation of the CAR upon binding to an antigen.
EMBODIMENT 229 The method of Embodiment 228, wherein the CAR is a first generation CAR in which the primary signaling domain is a member selected from the group consisting of CD3zeta, CD28, 4-1BB, OX40, DAP10, ICOS, and a variant thereof.
EMBODIMENT 230 The method of Embodiment 228, wherein the CAR is a second generation CAR in which (i) the primary signaling domain is a member selected from the group consisting of CD3zeta, CD28, 4-1BB, OX40, DAP10, ICOS, and a variant thereof, and (ii) the co-stimulatory signaling domain is a different member selected from the group consisting of CD3zeta, CD28, 4-1BB, OX40, DAP10, ICOS, and a variant thereof.
EMBODIMENT 231 The method of any one of Embodiments 131-230, wherein the antigen is a tumor antigen or cancer antigen a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-llRa, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GMl, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B¹, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
EMBODIMENT 232 The method of any one of Embodiments 131-231, wherein the antigen comprises a neoantigen encoded by a tumor-specific mutated gene.
EMBODIMENT 233 The method of any one of Embodiments 131-232, wherein the side effect comprises cytokine release syndrome (CRS), inflammatory disorder, or autoimmune disorder.
EMBODIMENT 234 A modified cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, wherein expression or activity of PTPN2 in the cell is downregulated, to potentiate immunity of the modified cell.
EMBODIMENT 235 The modified cell of Embodiment 234, wherein the modified cell exhibits a mutation of (i) a first gene encoding PTPN2 or (ii) a second gene operatively linked to PTPN2, wherein the mutation inhibits the expression and/or activity of PTPN2.
EMBODIMENT 236 The modified cell of Embodiment 234, wherein the expression or activity of PTPN2 is transiently downregulated.
EMBODIMENT 237 The modified cell of Embodiment 236, wherein the expression or activity of PTPN2 is downregulated by a compound of any one of Embodiments 1-128.
EMBODIMENT 238 The modified cell of Embodiment 237, wherein the compound does not regulate site-specific recombination of a gene encoding PTPN2.
EMBODIMENT 239 The modified cell of Embodiment 237, wherein the compound does not affect editing of (i) the gene encoding PTPN2 or (ii) an additional gene operatively linked to PTPN2.
EMBODIMENT 240 The modified cell of Embodiment 237, wherein the compound is configured to bind PTPN2.
EMBODIMENT 241 The modified cell of Embodiment 240, wherein the compound exhibits binding specificity to PTPN2 in comparison to other tyrosine phosphatases.
EMBODIMENT 242 The modified cell of Embodiment 240, wherein the small molecule exhibits IC₅₀ of less than or equal to 5 μM for PTPN2.
EMBODIMENT 243 The modified cell of any one of Embodiments 234-242, wherein the modified cell comprises a compound of any one of Embodiments 1-128.
EMBODIMENT 244 The modified cell of any one of Embodiments 234-243, wherein the TFP comprises a TCR subunit that comprises (1) a TCR extracellular domain capable of specific binding to the antigen, and (2) an intracellular signaling domain, wherein the TFP forms a TCR complex.
EMBODIMENT 245 The modified cell of Embodiment 244, wherein the TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together.
EMBODIMENT 246 The modified cell of Embodiment 244, wherein the TCR intracellular domain comprising a stimulatory domain from an intracellular signaling domain of epsilon chain, delta chain, and/or a gamma chain of cluster of differentiation 3 (CD3).
EMBODIMENT 247 The modified cell of Embodiment 244, wherein the TCR intracellular domain comprising a stimulatory domain from an intracellular signaling domain of TCR alpha, or from an intracellular signaling domain of TCR beta.
EMBODIMENT 248 The modified cell of any one of Embodiments 234-247, wherein the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154.
EMBODIMENT 249 The modified cell of any one of Embodiments 234-248, wherein the TFP comprises a costimulatory domain.
EMBODIMENT 250 The modified cell of Embodiment 249, wherein the costimulatory domain of the TFP is selected from the group consisting of: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
EMBODIMENT 251 The modified cell of any one of Embodiments 234-250, wherein the modified cell is a modified lymphoid cell.
EMBODIMENT 252 The modified cell of Embodiment 251, wherein the modified lymphoid cell is a variant of a member selected from the group consisting of: a T cell, B cell, NK cell, KHYG cell, T helper cell, regulatory T cell, memory T cell, tumor infiltration T cell (TIL), antigen presenting cell, and dendritic cell.
EMBODIMENT 253 The modified cell of Embodiment 251, wherein the modified lymphoid cell is a variant of a member selected from the group consisting of: a CD4+ T cell, a CD8+ T cell, and a CD4+ and CD8+ T cell.

The following examples are given for the purpose of illustrating various embodiments of the disclosure and are not meant to limit the present disclosure in any fashion. The present examples, along with the methods and compositions described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### EXAMPLES

The following examples are provided for illustrative purposes only and not to limit the scope of the claims provided herein.

As used herein, the following abbreviations, unless otherwise indicated, shall be understood to have the following meanings:

| | |
|---|---|
| ACN or MeCN | acetonitrile |
| AcOH | acetic acid |
| Ac | acetyl |
| BINAP | 2,2′-bis(diphenylphosphino)-1,1′-binaphthalene |
| Bn | benzyl |
| BOC or Boc | tert-butyl carbamate |
| i-Bu | iso-butyl |
| t-Bu | tert-butyl |
| DCM | dichloromethane (CH₂Cl₂) |
| DIBAL-H | diisobutylaluminum hydride |
| DIPEA or DIEA | diisopropylethylamine |
| DMAP | 4-(N,N-dimethylamino)pyridine |
| DME | 1,2-dimethoxyethane |
| DMF | N,N-dimethylformamide |
| DMA | N,N-dimethylacetamide |
| DMSO | dimethylsulfoxide |
| Dppf or dppf | 1,1′-bis(diphenylphosphino)ferrocene |
| EDC or EDCI | N-(3-dimethylaminopropyl)-N′-ethylcarbodiimide |
| | hydrochloride |
| eq | equivalent(s) |
| Et | ethyl |
| Et₂O | diethyl ether |
| EtOH | ethanol |
| EtOAc | ethyl acetate |
| HPLC | high performance liquid chromatography |
| KHMDS | potassium bis(trimethylsilyl)amide |
| NaHMDS | sodium bis(trimethylsilyl)amide |
| LIHMDS | lithium bis(trimethylsilyl)amide |
| LAH | lithium aluminum anhydride |
| LCMS | liquid chromatography mass spectrometry |
| Me | methyl |
| MeOH | methanol |
| MS | mass spectroscopy |
| Ms | mesyl |
| NMR | nuclear magnetic resonance |
| Ph | phenyl |
| iPr/i-Pr | iso-propyl |
| RP-HPLC | reverse-phase high-pressure liquid chromatography |
| rt | room temperature |
| TBS | tert-butyldimethylsilyl |
| TEA | triethylamine |
| TFA | trifluoroacetic acid |
| THF | tetrahydrofuran |
| TLC | thin layer chromatography |
| TMS | trimethylsilyl |
| TsOH/p-TsOH | p-toluenesulfonic acid |

### Example 1.1: Synthesis of 5-(6-hydroxy-2-(isopentylamino)quinolin-7-yl)-1,2,5-thiadiazolidin-3-one 1,1-dioxide (Compound 113)

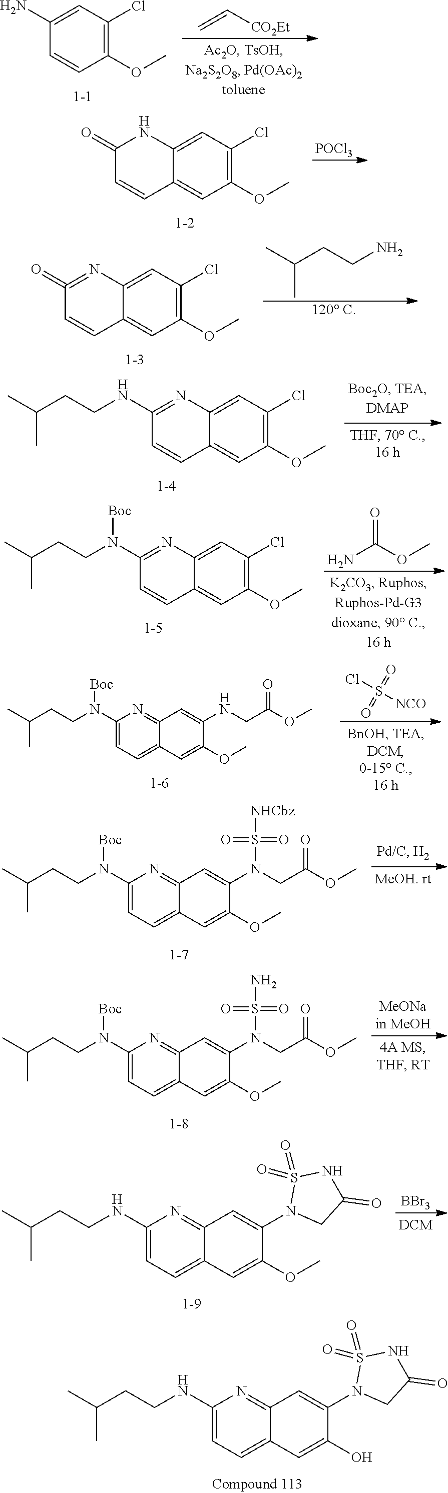

To a mixture of compound 1-1 (10 g, 63.7 mmol, 1.0 eq.) and ethyl acrylate (31.8 g, 318.5 mmol, 5.0 eq.) in toluene (500 mL) was added Ac₂O (13 g, 127.4 mmol, 2.0 eq.) and TsOH·H₂O (12 g, 63.7 mmol, 1.0 eq.), followed by the addition of Na₂S₂O₈ (45.4 g, 191 mmol, 3.0 eq.) and Pd(OAc)₂(1.41 g, 6.37 mmol, 0.1 eq.). The reaction mixture was stirred at 100° C. under N₂ for 48 h. The reaction mixture was filtered and concentrated under reduced pressure. The residue was purified by column chromatography on silica gel eluting with petroleum ether/ethyl acetate (1/0-0/1) to obtain compound 1-2 (2.5 g). ESI-MS m/z: 210.0 [M+H]⁺.

To a mixture of compound 1-2 (2 g, 9.2 mmol, 1.0 eq.) in POCl₃ (20 mL) was stirred at 100° C. for 16 h under N₂. The reaction mixture was concentrated under reduced pressure. The residue was purified by column chromatography on silica gel eluting with eluting with petroleum ether/ethyl acetate (1/0-3/1) to obtain compound 1-3 (1.2 g). ESI-MS m/z: 228.0 [M+H]⁺.

To a mixture of compound 1-3 (0.8 g, 3.52 mmol, 1eq.) in 3-methylbutan-1-amine (10 mL) was added TEA (1.78 g, 17.62 mmol, 5 eq.). The reaction mixture was stirred at 120° C. under N₂ for 12 h on microwave. The reaction mixture was concentrated under reduced pressure. The residue was purified by column chromatography on silica gel eluting with petroleum ether/ethyl acetate (1/0-4/1) to give compound 1-4 (0.7 g). ESI-MS m/z: 279.1 [M+H]⁺.

To a mixture of compound 1-4 (0.3 g, 1.08 mmol, 1.0 eq.), Boc₂O (353 g, 1.62 mmol, 1.5 eq.) and TEA (327 g, 3.24 mmol, 3.0 eq.) in THF (10 mL) was added DMAP (132 mg, 1.08 mmol, 1.0 eq.) under N₂. The reaction mixture was stirred at 70° C. under N₂ for 16 h. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-10/1) to obtain compound 1-5 (330 mg).

To a mixture of compound 1-5 (330 mg, 0.87 mmol, 1.0 eq.), methyl glycinate (160 mg, 1.31 mmol, 1.5 eq.), K₂CO₃ (482 mg, 3.49 mmol, 4.0 eq.) and Ruphos (202 mg, 0.44 mmol, 0.5 eq.) in dioxane (15 mL) was added Ruphos Pd G₃ (73 mg, 0.087 mmol, 0.1 eq.) under N₂. The reaction mixture was stirred at 100° C. under N₂ for 16 h. The reaction mixture was diluted with DCM (30 mL) and washed with H₂O (2×30 mL), dried over anhydrous Na₂SO₄, filtered and concentrated under reduced pressure. The residue was purified by column chromatography on silica gel eluting with petroleum ether/ethyl acetate (1/0-2/1) to obtain compound 1-6 (250 mg). ESI-MS m/z: 432.2 [M+H]⁺.

To a mixture of compound 1-6 (250 mg, 0.58 mmol, 1.0 eq.) and BnOH (124 mg, 1.16 mmol, 2.0 eq.) in DCM (10 mL) was added sulfurisocyanatidic chloride (164 mg, 1.16 mmol, 2.0 eq.). The reaction mixture was stirred at room temperature under N₂ for 1 h. Then TEA (293 mg, 2.9 mmol, 5.0 eq.) was added to the mixture. The reaction mixture was stirred at room temperature for 16 h under N₂. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-1/1) to obtain compound 1-7 (200 mg). ESI-MS m/z: 645.4[M+H]⁺.

To a mixture of compound 1-7 (180 mg, 0.3 mmol, 1.0 eq.) in MeOH (5 mL) was added Pd/C (18 mg, 10%, 0.1 eq.). The reaction mixture was stirred at room temperature under H₂ for 2 h. The reaction mixture was filtered and concentrated under reduced pressure to obtain compound 1-8 (150 mg), which was used in the next step without further purification. ESI-MS m/z: 511.1 [M+H]⁺.

To a mixture of compound 1-8 (120 mg, 0.235 mmol, 1.0 eq.) in THF (5 mL) was added NaOMe (25 mg, 0.47 mmol, 2.0 eq.) under N₂. After addition, the mixture was stirred at room temperature for 2 h under N₂. The mixture was concentrated under reduced pressure. The residue was purified by column chromatography on silica gel eluting with eluting with DCM/MeOH (1/0-5:1, v/v) to obtain compound 1-9 (80 mg). ESI-MS m/z: 479.1 [M+H]⁺.

To a mixture of compound 1-9 (80 mg, 0.17 mmol, 1.0 eq.) in DCM (2 mL) was added BBr₃ (0.5 mL) at −78° C. After addition, the mixture was stirred at 35° C. for 4 h under N₂. The mixture was quenched by NH₃·H₂O dropwise at −78° C. to pH-8. The reaction mixture was filtered and concentrated to give crude product, which was purified by prep-HPLC (NH₃·H₂O) to afford Compound 113 (5.44 mg). ESI-MS m/z: 365.0 [M+H]⁺; ¹H NMR (400 MHz, CD₃OD) S 8.24 (s, 1H), 8.09 (s, 1H), 7.16 (s, 1H), 6.86(s, 1H), 4.33(s, 2H), 3.44 (s, 2H), 1.74-1.70 (m, 1H), 1.54 (s, 2H), 0.95 (d, J=8.0 Hz, 611).

### Example 1.2: Synthesis of 5-(6-hydroxy-2-(isopentylamino)quinolin-7-yl)isothiazol-3(2H)-one 1,1-dioxide (Compound 106)

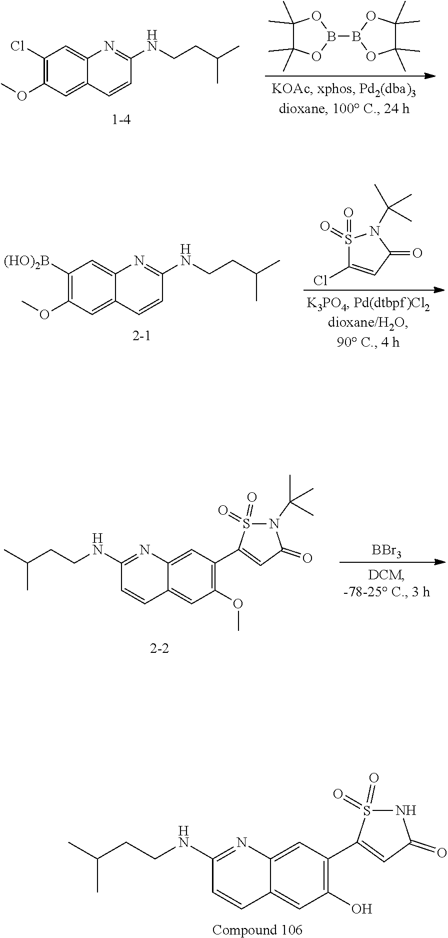

To a mixture of compound 1-4 (200 mg, 0.72 mmol, 1.0 eq.), 4,4,4′,4′,5,5,5′,5′-octamethyl-2,2′-bi(1,3,2-dioxaborolane) (365 mg, 1.44 mmol, 2.0 eq.) and KOAc (212 mg, 2.16 mmol, 3.0 eq.) in dioxane (10 mL) was added xphos (153 mg, 0.36 mmol, 0.5 eq.) and Pd₂(dba)₃ under N₂. The reaction mixture was stirred at 100° C. under N₂ for 16 h. The reaction mixture was filtered and concentrated to afford compound 2-1, which was used in the next step without further purification. ESI-MS m/z: 288.8 [M+H]⁺.

To a mixture of compound 2-1 (80 mg, 0.28 mmol, 1.0 eq.), compound 6 (124 mg, 0.56 mmol, 2.0 eq.) and K₃PO₄ (178 mg, 0.84 mmol, 3.0 eq.) in Dioxane (5 mL) and H₂O (1 mL) was added Pd(dtbpf)Cl₂ (20 mg, 0.028 mmol, 0.1 eq.) under N₂. The reaction mixture was stirred at 90° C. under N₂ for 3 h. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-2/1) to obtain compound 2-2 (60 mg). ESI-MS m/z: 432.1 [M+H]⁺.

To a mixture of compound 2-2 (60 mg, 0.14 mmol, 1.0 eq.) in DCM (2 mL) was added BBr₃ (0.5 mL) at −78° C. After addition, the mixture was stirred at 35° C. for 4 h under N₂. The mixture was quenched by NH₃·H₂O dropwise at −78° C. to pH-8. The reaction mixture was filtered and concentrated to give crude product, which was purified by prep-HPLC (HCOOH) to afford Compound 106 (4.21 mg). ESI-MS m/z: 362.0 [M+H]⁺; 1H NMR (400 MHz, CD₃OD) δ 8.53 (s, 1H), 8.10 (d, J=12.0 Hz, 1H), 7.43 (s, 1H), 7.36 (s, 1H), 7.08 (d, J=8.4 Hz, 1H), 3.56-3.47 (m, 2H), 1.79-1.73 (m, 1H), 1.70-1.64 (m, 2H), 1.02 (d, J=8.0 Hz, 6H).

### Example 1.3: Synthesis of 5-(7-hydroxy-3-isopentyl-1-methyl-2-oxo-1,2,3,4-tetrahydroquinazolin-6-yl)isothiazol-3(2H)-one 1,1-dioxide (Compound 108)

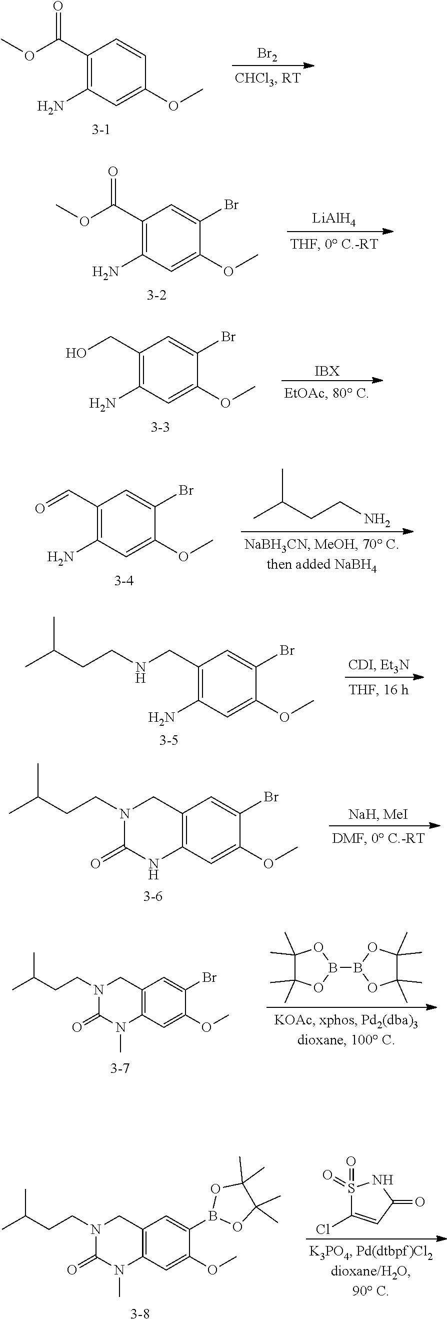

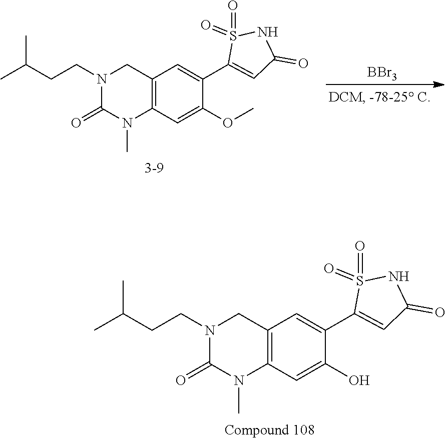

To a mixture of compound 3-1 (10 g, 55.2 mmol, 1 eq.) in CHCl₃ (250 mL) was added a solution of Br₂ (2.83 mL, 55.2 mmol, 1.0 eq.) in CHCl₃ (10 mL) at 0° C. under N₂. After addition, the reaction mixture was stirred at room temperature under N₂ for 2 h. The reaction mixture quenched with Na₂S₂O₃ solution (200 mL) and extracted with DCM (2×200 mL). The combined organic layers were dried over anhydrous Na₂SO₄, filtered and concentrated under reduced pressure. The residue was purified by column chromatography on silica gel eluting with eluting with EtOAc/petroleum ether (0/1-1/5, v/v) to obtain compound 2 (10.4 g). 1H NMR (400 MHz, DMSO-d6): δ 7.79 (s, 1H), 6.85 (s, 2H), 6.45 (s, 1H), 3.80 (s, 3H), 3.75 (s, 3H).

To a mixture of compound 3-2 (10.5 g, 40.4 mmol, 1.0 eq.) in THF (100 mL) was added LiAlH₄ (80 mL, 80.8 mmol, 2 eq., 1 M in THF) at 0° C. under N₂. The mixture was stirred at 0-10° C. for 2 h under N₂. The reaction mixture was quenched with sat. Na₂SO₄ solution (15 mL), then diluted with DCM (100 mL). The mixture was dried over anhydrous Na₂SO₄, filtered and concentrated under reduced pressure to obtain crude compound 3-3 (9.2 g), which was used for the next step without further purification. ¹H NMR (400 MHz, DMSO-d6): δ 7.15 (s, 1H), 6.38 (s, 1H), 5.11 (s, 2H), 4.96 (t, J=5.5 Hz, 1H), 4.29 (d, J=5.5 Hz, 2H), 3.72 (s, 3H).

To a solution of compound 3-3 (9.2 g, 39.8 mmol, 1 eq.) in EtOAc (90 mL) was added IBX (22.3 g, 79.6 mmol, 2 eq.) slowly at 0° C. The reaction mixture was stirred 80° C. for 2 h. The mixture was filtered and the filter cake was washed with EtOAc (100 mL). The filtrate was concentrated under reduced pressure to obtain compound 3-4 (11.3 g), which was used for the next step without further purification.

To a mixture of compound 3-4 (5 g, 21.83 mmol, 1 eq.) in MeOH (50 mL) was added 3-methylbutan-1-amine (3.8 g, 43.67 mmol, 2.0 eq.) and AcOH (3 drops), then added NaBH₃CN (6.9 g, 109. 15 mmol). The mixture was stirred at 70° C. for 2 h under N₂. Then NaBH₄ (1.66 g, 43.67 mmol, 2.0 eq.) was added to the mixture. The mixture was stirred at RT for 1 h under N₂. The mixture was quenched with aq. NH₄Cl solution (50 mL) and extracted with DCM (3×50 mL). The combined organic layers were dried over anhydrous Na₂SO₄, filtered and concentrated under reduced pressure. The residue was purified by column chromatography on silica gel eluting with eluting with DCM/MeOH (1/0-10/1, v/v) to obtain compound 3-5 (3.7 g). ESI-MS m/z: 302.9 [M+3]⁺.

To a mixture of compound 3-5 (3.2 g, 10.7 mmol, 1 eq.) in THF (50 mL) was added Et₃N (4.32 g, 42.8 mmol, 4 eq.) and CDI (5.2 g, 32.1 mmol, 3 eq.). The mixture was stirred at RT for 16 h under N₂. The mixture was quenched with water (80 mL) and extracted with DCM (3×80 mL). The combined organic layers were dried over anhydrous Na₂SO₄, filtered and concentrated under reduced pressure. The residue was purified by column chromatography on silica gel eluting with eluting with PE/EtOAc (1/0-1/2, v/v) to obtain compound 3-6 (2.5 g). ESI-MS m/z: 328.9 [M+3]⁺.

To a mixture of compound 3-6 (1.5 g, 4.6 mmol, 1 eq.) in DMF (10 mL) was added NaH (368 mg, 9.2 mmol, 2.0 eq., 60% in mineral oil) at 0° C. under N₂. The mixture was stirred at 0° C. for 0.5 h under N₂. Mel (0.98 g, 6.9 mmol, 1.5 eq.) was added to the reaction mixture at 0° C. After addition, the mixture was stirred at 0° C.-RT for 1 h under N₂. The mixture was quenched with aq. NH₄Cl solution (25 mL) and extracted with EtOAc (3×25 mL). The combined organic layers were washed with brine (3×100 mL), dried over anhydrous Na₂SO₄, filtered and concentrated under reduced pressure. The residue was purified by column chromatography on silica gel eluting with eluting with petroleum ether/EtOAc (1/0-1/1, v/v) to obtain compound 3-7 (1 g). ESI-MS m/z: 340.9 [M+H]⁺.

To a mixture of compound 3-7 (50 mg, 0.147 mmol, 1.0 eq.), 4,4,4′,4′,5,5,5′,5′-octamethyl-2,2′-bi(1,3,2-dioxaborolane) (112 mg, 0.441 mmol, 3.0 eq.) and KOAc (43 mg, 0.441 mmol, 3.0 eq.) in dioxane (3 mL) was added xphos (14 mg, 0.0294 mmol, 0.2 eq.) and Pd₂(dba)₃ (26.9 mg, 0.0294 mmol, 0.2 eq.) under N₂. The reaction mixture was stirred at 100° C. under N₂ for 16 h. The reaction mixture was filtered and the filter cake was washed with THF (50 mL). The filtrate was concentrated under reduced pressure to give crude product 3-8 (57 mg), which was used for the next step without further purification. ESI-MS m/z: 389.1 [M+H]⁺.

To a mixture of compound 3-8 (57 mg, 0.147 mmol, 1.0 eq.), 5-chloroisothiazol-3(2H)-one 1,1-dioxide (33 mg, 0.147 mmol, 1.0 eq.) and K₃PO₄ (94 mg, 0.441 mmol, 3.0 eq.) in Dioxane (2.5 mL) and H₂O (0.25 mL) was added Pd(dtbpf)Cl₂ (10 mg, 0.0147 mmol, 0.1 eq.) under N₂. The reaction mixture was stirred at 90° C. under N₂ for 16 h. The reaction mixture was diluted with DCM (20 mL) and washed with H₂O (2×20 mL), dried over anhydrous Na₂SO₄, filtered and concentrated under reduced pressure. The residue was purified by preparative TLC separation with petroleum ether/ethyl acetate=1/1 to obtain compound 3-9 (40 mg). ESI-MS m/z: 450.2 [M+H]⁺.

To a mixture of compound 3-9 (40 mg, 0.089 mmol, 1 eq.) in DCM (3 mL) was added BBr₃ (1 mL) at −78° C. under N₂. The mixture was stirred at room temperature for 1 h under N₂. The mixture was cooled to −78° C. and quenched with MeOH (10 mL), then adjusted to pH=8-9 with NH₃·H₂O. The mixture was concentrated under reduced pressure. The residue was resolved in DMF (4 mL) and filtered. The filtrate was purified by basic prep-HPLC separation to obtain Compound 108 (3.5 mg). ESI-MS m/z: 380.0 [M+H]⁺; ¹H NMR (400 MHz, CD₃OD): δ 7.70 (s, 1H), 6.95 (s, 1H), 6.53 (s, 1H), 4.38 (s, 2H), 3.48-3.42 (m, 2H), 3.26 (s, 311), 1.64-1.46 (m, 311), 0.96 (d, J=6.5 Hz, 6H).

### Example 1.4: Synthesis of 5-(7-hydroxy-3-isopentyl-1-methyl-2-oxo-1,2,3,4-tetrahydroquinazolin-6-yl)isothiazolidin-3-one 1,1-dioxide (Compound 111)

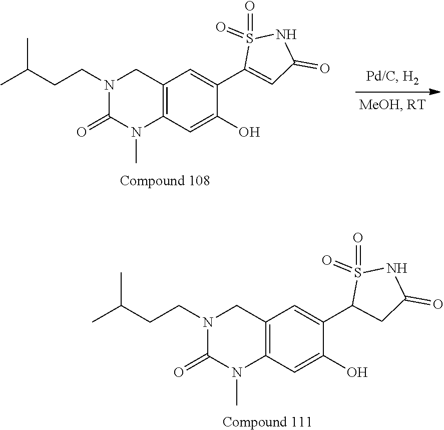

To a mixture of Compound 108 (70 mg, 0.18 mmol, 1.0 eq.) in MeOH (2 mL) was added Pd/C (20 mg, 10%). The reaction mixture was stirred at room temperature under H₂ (30 psi) for 16 h. LCMS showed that the desired product was formed. The reaction mixture was filtered and the filter cake was washed with DCM/MeOH (20 mL, 1/1). The filtrate was concentrated under reduced pressure to give crude product, which was purified by basic prep-HPLC separation to afford Compound 111 (3.11 mg). ESI-MS m/z: 382.2 [M+H]⁺; ¹H NMR (400 MHz, CD₃OD): δ 7.00 (s, 1H), 6.43 (s, 1H), 5.26-5.03 (m, 1H), 4.31 (s, 2H), 3.47-3.39 (m, 211), 3.23-3.20 (m, 411), 3.06-2.97 (m, 1H), 1.62-1.44 (m, 311), 0.95 (d, J=6.5 Hz, 6H).

### Example 1.5: Synthesis of 5-(7-hydroxy-1-methyl-2-oxo-3-(phenethylamino)-1,2-dihydroquinolin-6-yl)-1,2,5-thiadiazolidin-3-one 1,1-dioxide (Compound 123)

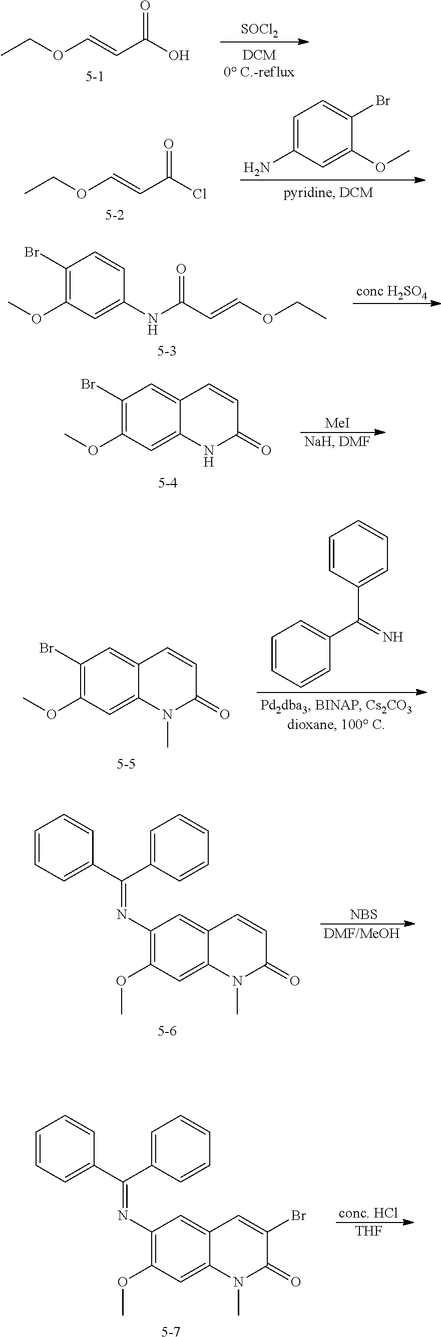

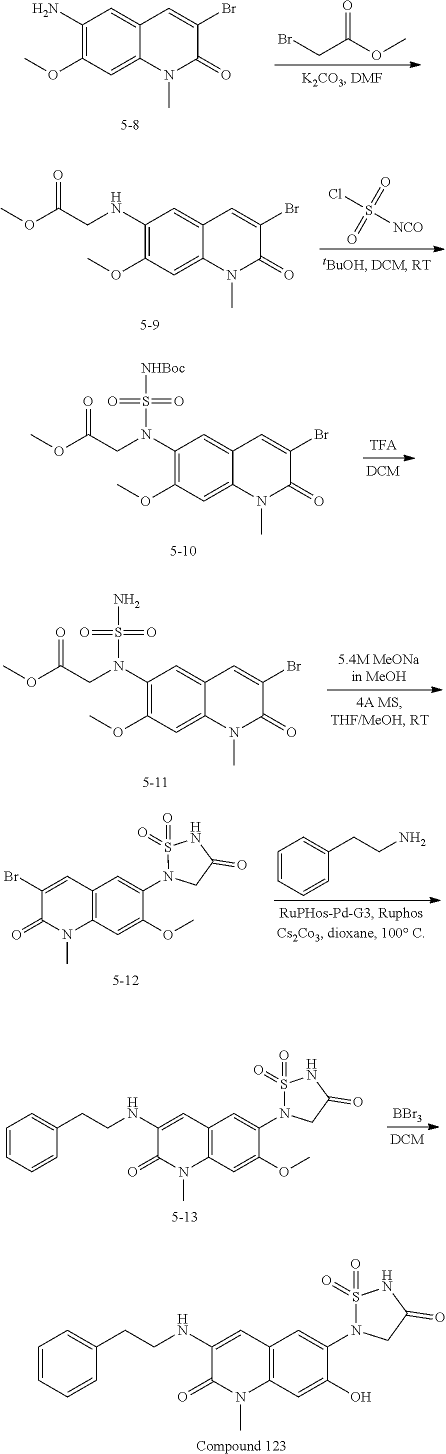

To a mixture of compound 5-1 (5 g, 43.05 mmol, 1 eq.) in DCM (170 mL) was added SOCl₂ (4.7 mL, 64.59 mmol, 1.5 eq.) at 0° C. under N₂. The reaction mixture was stirred at reflux under N₂ for 3 h. The reaction mixture was concentrated under reduced pressure to obtain crude compound 5-2 (5.77 g), which was used for the next step without further purification.

To a mixture of compound 5-2 (5.77 g, 43.05 mmol, 1.3 eq.) and 4-bromo-3-methoxyaniline (6.65 g, 33.11 mmol, 1 eq.) in DCM (50 mL) was added pyridine (5.33 mL, 66.22 mmol, 2 eq.) at 0° C. under N₂. The mixture was stirred at room temperature for 3 h under N₂. The reaction mixture was diluted with DCM (100 mL) and washed with sat. NH₄Cl solution (3×150 mL). The organic layer was dried over anhydrous Na₂SO₄, filtered and concentrated under reduced pressure. The residue was purified by column chromatography on silica gel eluting with eluting with DCM/petroleum ether (0/1-1/0, v/v) to obtain compound 5-3 (9.9 g).

To a solution of H₂SO₄ (70 mL) was added compound 5-3 (8.9 g, 29.76 mmol) slowly at 0° C. The reaction mixture was stirred 0° C. for 20 min. The reaction mixture was added to ice water and lots of solids were precipitated out. The mixture was filtered and the filter cake was washed with H₂O (200 mL). The solids was collected and dried under reduced pressure to obtain compound 5-4 (6.7 g), which was used for the next step without further purification. ESI-MS m/z: 253.9 [M+H]⁺.

To a mixture of compound 5-4 (2.5 g, 9.88 mmol, 1 eq.) in DMF (50 mL) was added NaH (790 mg, 19.76 mmol, 2.0 eq., 60% in mineral oil) at 0° C. under N₂. The mixture was stirred at 0° C. for 0.5 h under N₂. Mel (0.92 mL, 14.82 mmol, 1.5 eq.) was added to the reaction mixture at 0° C. After addition, the mixture was stirred at 0° C.-RT for 2 h under N₂. The mixture was diluted with DCM (100 mL) and washed with brine (3×100 mL). The organic layer was dried over anhydrous Na₂SO₄, filtered and concentrated under reduced pressure. The residue was purified by column chromatography on silica gel eluting with eluting with petroleum ether/EtOAc (1/0-0/1, v/v) to obtain compound 5-5 (1.9 g). ESI-MS m/z: 267.9 [M+H]⁺; ¹H NMR (400 MHz, DMSO-d6): δ 7.98 (s, 1H), 7.79 (d, J=8.0 Hz, 1H), 7.01 (s, 1H), 6.48 (d, J=8.0 Hz, 1H), 4.01 (s, 3H), 3.63 (s, 3H).

To a mixture of compound 5-5 (1.9 g, 7.11 mmol, 1 eq.) and diphenylmethanimine (3.86 g, 21.35 mmol, 3 mmol) in dioxane (30 mL) was added BINAP (443 mg, 0.71 mmol, 0.1 eq.), Cs₂CO₃ (6.96 g, 21.35 mmol, 3 eq.) and Pd₂dba₃ (652 mg, 0.71 mmol, 0.1 eq.) under N₂. The mixture was stirred at 100° C. for 16 h under N₂. The mixture was concentrated under reduced pressure. The residue was purified by column chromatography on silica gel eluting with eluting with petroleum ether/EtOAc (1/0-0/1, v/v) to obtain compound 5-6 (2.9 g). ESI-MS m/z: 369.0 [M+H]⁺.

To a mixture of compound 5-6 (2.4 g, 6.52 mmol, 1 eq.) in DMF (10 mL) and MeOH (5 mL) was added NBS (1.39 g, 7.83 mmol, 1.2 eq.). The mixture was stirred at room temperature for 1 h under N₂. The mixture was diluted with EtOAc (100 mL) and washed with brine (3×100 mL). The organic layer was dried over anhydrous Na₂SO₄, filtered and concentrated under reduced pressure to obtain crude compound 5-7 (2.91 g), which was used for the next step without further purification.

To a mixture of compound 5-7 (2.91 g, 6.52 mmol, 1 eq.) in THF (20 mL) was added conc. HCl solution (2 mL). The mixture was stirred at room temperature for 0.5 h. The mixture was adjusted with pH=8-9 with aq. NaHCO₃ solution and extracted with DCM (3×100 mL). The combined organic layers were dried over anhydrous Na₂SO₄, filtered and concentrated under reduced pressure. The residue was purified by column chromatography on silica gel eluting with eluting with petroleum ether/EtOAc (1/0-0/1, v/v) to DCM/EtOAc (1/1, v/v) to obtain compound 5-8 (2.1 g).

To a mixture of compound 5-8 (2.1 g, 7.44 mmol, 1 eq.) in DMF (20 mL) was added K₂CO₃ (3.083 g, 22.34 mmol, 3 eq.) and methyl 2-bromoacetate (2.28 g, 14.89 mmol, 2 eq.). The mixture was stirred at 60° C. for 2 h under N₂. The mixture was diluted with DCM (100 mL) and washed with brine (3×100 mL). The organic layer was dried over anhydrous Na₂SO₄, filtered and concentrated under reduced pressure. The residue was purified by column chromatography on silica gel eluting with eluting with petroleum ether/EtOAc (1/0-0/1, v/v) to DCM/EtOAc (1/1, v/v) to obtain compound 5-9 (1.8 g). ¹H NMR (400 MHz, DMSO-d6): δ 8.24 (s, 1H), 6.94 (s, 1H), 6.68 (s, 1H), 5.52-5.45 (m, 1H), 4.00 (s, 3H), 3.98 (d, J=8.0 Hz, 2H), 3.71 (s, 3H), 3.63 (s, 3H).

To a mixture of sulfurisocyanatidic chloride (3.2 g, 22.6 mmol, 10.0 eq.) in DCM (15 mL) was added tBuOH (1.68 g, 22.6 mmol, 10 eq.) at 0° C. under N₂. The reaction mixture was stirred at room temperature under N₂ for 0.5 h. Then a solution of compound 5-9 (800 mg, 2.26 mmol, 1 eq.) and TEA (6.25 mL, 45.2 mmol, 20 eq.) in DCM (15 mL) was added to the mixture at 0° C. under N₂. The reaction mixture was stirred at room temperature for 2 h under N₂. The reaction mixture was quenched with H₂O (80 mL) and extracted with DCM (3×80 mL). The combined organic layers were dried over anhydrous Na₂SO₄, filtered and concentrated under reduced pressure to obtain crude compound 5-10 (1.20 g), which was used for the next step without further purification.

To a mixture of compound 5-10 (1.20 g, 2.26 mmol, 1 eq.) in DCM (10 mL) was added TFA (10 mL). The mixture was stirred at room temperature for 4 h. The mixture was adjusted with pH=8-9 with aq. NaHCO₃ solution and extracted with DCM (3×100 mL). The combined organic layers were dried over anhydrous Na₂SO₄, filtered and concentrated under reduced pressure. The residue was triturated with petroleum ether/EtOAc/MeOH (20 mL, 1/3/0.25), then the mixture was filtered and the filter cake was washed with petroleum ether/EtOAc/MeOH (3 mL, 1/3/0.25). The solid was collected and dried under reduced pressure to obtain compound 5-11 (380 mg). ESI-MS m/z: 435.9 [M+3]⁺.

To a mixture of compound 5-11 (380 mg, 0.877 mmol, 1.0 eq.) and 4A MS (800 mg) in THF (10 mL) and MeOH (10 mL) was added NaOMe solution (0.66 mL, 3.51 mmol, 4.0 eq., 5.4 M in MeOH) under N₂. After addition, the mixture was stirred at room temperature for 2 h under N₂. The mixture was concentrated under reduced pressure. The residue was purified by column chromatography on silica gel eluting with eluting with EtOAc/MeOH (1/0-3/1, v/v) to obtain compound 5-12 (370 mg). ESI-MS m/z: 403.8 [M+H]⁺.

To a mixture of compound 5-12 (100 mg, 0.24 mmol, 1.0 eq.), 2-phenylethan-1-amine (91 mg, 0.74 mmol, 3 eq.), Cs₂CO₃ (244 mg, 0.74 mmol, 3 eq.) and Ruphos (35 mg, 0.074 mmol, 0.3 eq.) in dioxane (5 mL) was added Ruphos Pd G₃ (63 mg, 0.074 mmol, 0.3 eq.) under N₂. The reaction mixture was stirred at 100° C. under N₂ for 48 h. The reaction mixture was concentrated under reduced pressure. The residue was purified by column chromatography on silica gel eluting with EtOAc/MeOH (1/0-3/1, v/v) to obtain compound 5-13 (200 mg). ESI-MS m/z: 441.0 [M−H]⁻.

To a mixture of compound 5-13 (200 mg, 0.45 mmol, 1 eq.) in DCM (2 mL) was added BBr₃ (2 mL) at −78° C. under N₂. The mixture was stirred at 35° C. for 1 h under N₂. The mixture was cooled to −78° C. and quenched with MeOH (5 mL), then adjusted to pH=8-9 with NH₃·H₂O. The mixture was concentrated under reduced pressure. The residue was resolved in DMF (4 mL) and filtered. The filtrate was purified by basic prep-HPLC separation to afford Compound 123 (7 mg). ESI-MS m/z: 427.0 [M−H]⁻; ¹H NMR (400 MHz, DMSO-d6): δ 7.56 (s, 1H), 7.32-7.21 (m, 5H), 6.84 (s, 1H), 6.59 (s, 1H), 5.36 (t, J=6.0 Hz, 1H), 4.06 (s, 2H), 3.59 (s, 3H), 3.38-3.33 (m, 2H), 2.90 (t, J=7.2 Hz, 2H).

### Example 1.6: Synthesis of 5-(6-hydroxy-2-isopentyl-3-oxo-1,2,3,4-tetrahydroisoquinolin-7-yl)-1,2,5-thiadiazolidin-3-one 1,1-dioxide (Compound 119)

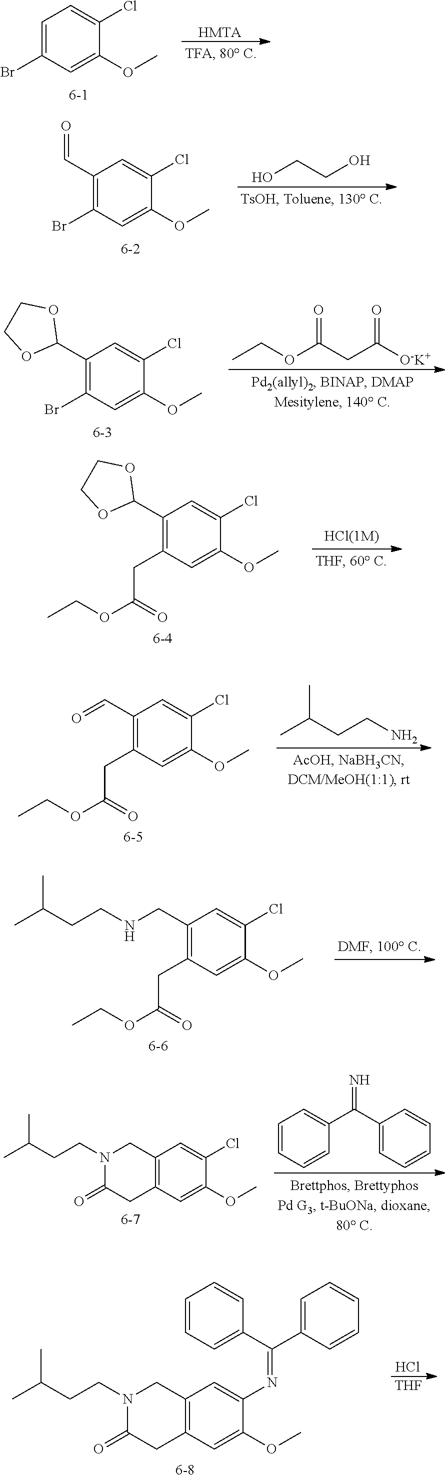

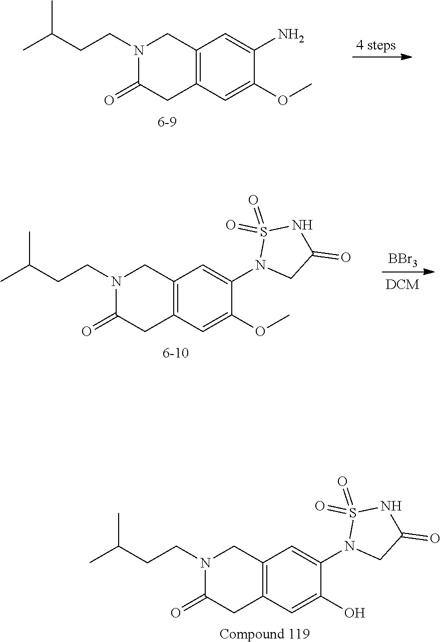

To a solution of compound 6-1 (50.0 g, 226.2 mmol, 1.0 eq.) in TFA (400 mL) was added HMTA (47.5 g, 339.3 mmol, 1.5 eq.) at 0° C. The reaction mixture was stirred at 80° C. under N₂ for 16 h. The reaction mixture was filtered and the filtrate was concentrated. Then diluted with H₂O (300 mL) and added with aqueous NaHCO₃ to pH=7, extracted with EA (400 mL) three times. The organic layer was dried over anhydrous Na₂SO₄, filtered and concentrated under reduced pressure to give compound 6-2 (39.0 g). ¹H NMR (400 MHz, DMSO-d6): δ 10.04 (s, 1H), 7.84 (s, 1H), 7.55 (s, 1H), 4.01 (s, 3H).

To a solution of compound 6-2 (14.0 g, 56.2 mmol, 1.0 eq.) in Toluene (250 mL) was added Ethylene glycol (17.4 g, 281.0 mmol, 5 eq.) and TsOH·H₂O (5.34 g, 28.1 mmol, 0.5 eq.). The reaction mixture was stirred at 130° C. under N₂ for 16 h. The reaction mixture was concentrated, then diluted with H₂O (200 mL) and extracted with EA (200 mL) three times. The organic layer was dried over anhydrous Na₂SO₄, filtered and concentrated under reduced pressure. The residue was purified by column chromatography on silica gel eluting with petroleum ether/ethyl acetate (1/0-5/1) to obtain compound 6-3 (13.0 g).

To a solution of compound 6-3 (2.0 g, 6.85 mmol, 1.0 eq.), Ethyl potassium malonate (1.4 g, 8.22 mmol, 1.2 eq.), BINAP (256 mg, 0.41 mmol, 0.06 eq.), DMAP (84 mg, 0.68 mmol, 0.1 eq.) in Mesitylene (80 mL) was added Pd₂(allyl)₂ (50 mg, 0.137 mmol, 0.02 eq.). The reaction mixture was stirred at 140° C. under N₂ for 2 days. The reaction mixture was concentrated, then diluted with H₂O (50 mL) and extracted with EA (50 mL) three times. The organic layer was dried over anhydrous Na₂SO₄, filtered and concentrated under reduced pressure. The residue was purified by column chromatography on silica gel eluting with petroleum ether/ethyl acetate (1/0-5/1) to obtain compound 6-4 (700 mg). ESI-MS m/z: 301.0 [M+H]⁺.

To a solution of compound 6-4 (5.0 g, 16.7 mmol, 1.0 eq.) in THF (50 mL) was added HCl(1 M, 16.7 mL, 16.7 mmol, 1.0 eq.). The reaction mixture was stirred at 60° C. for 3 h. The reaction mixture was diluted with H₂O (100 mL) and extracted with EA (100 mL) three times. The organic layer was dried over anhydrous Na₂SO₄, filtered and concentrated under reduced pressure. The residue was purified by column chromatography on silica gel eluting with petroleum ether/ethyl acetate (1/0-3/1) to obtain compound 6-5 (3.1 g). ESI-MS m/z: 257.0 [M+H]⁺.

To a solution of compound 6-5 (1.0 g, 3.91 mmol, 1.0 eq.) in DCM/MeOH (10 mL/10 mL) was added isoamylamine (374 mg, 4.30 mmol, 1.1 eq.) and AcOH (3 drops). The reaction mixture was stirred at rt for 1 h. Then NaBH₃CN (493 mg, 7.82 mmol, 2.0 eq.) was added to the solution and stirred at rt for 2 h. The reaction mixture was concentrated under reduced pressure. The residue was purified by column chromatography on silica gel eluting with petroleum ether/ethyl acetate (1/0-3/1) to obtain compound 6-6 (780 mg). ESI-MS m/z: 328.0 [M+H]⁺.

A solution of compound 6-6 (780 mg, 2.77 mmol, 1.0 eq.) in DMF (10 mL) was stirred at 100° C. for 6 h. The reaction mixture was diluted with brine (50 mL) and extracted with EA (30 mL) three times. The organic layer was dried over anhydrous Na₂SO₄, filtered and concentrated under reduced pressure. The residue was purified by column chromatography on silica gel eluting with petroleum ether/ethyl acetate (1/0-0/1) to obtain compound 6-7 (600 mg). ESI-MS m/z: 282.0 [M+H]⁺.

A mixture of compound 6-7 (600 mg, 2.135 mmol, 1.0 eq.) and compound 2 (387 mg, 2.135 mmol, 1.0 eq.), Brettphos (228 mg, 0.427 mmol, 0.2 eq.), Brettphos Pd G₃ (192 mg, 0.213 mmol, 0.1 eq.), t-BuONa (615 mg, 6.405 mmol, 3.0 eq.) in dioxane (7 mL) was stirred at 80° C. under N₂ for 16 h in a sealed tube. The reaction mixture was diluted with EA (20 mL) and washed with H₂O (40 mL), dried over anhydrous Na₂SO₄, filtered and concentrated under reduced pressure. The residue was purified by column chromatography on silica gel eluting with petroleum ether/ethyl acetate (1/0-1/1) to obtain compound 6-8 (420 mg). ESI-MS m/z: 427.1 [M+H]⁺.

To a mixture of compound 6-8 (420 mg, 0.986 mmol, 1.0 eq.) in THF (5 mL) was added HCl (172 mg, 4.93 mmol, 5.0 eq.). The mixture was stirred at room temperature for 3 h. The reaction mixture was added with aqueous NaHCO₃ (10 mL) to pH=7, then extracted with EtOAc (20 mL) and washed with H₂O (3×20 mL). The organic layer was dried over anhydrous Na₂SO₄, filtered and concentrated under reduced pressure. The residue was purified by column chromatography on silica gel eluting with petroleum ether/ethyl acetate (1/0-1/1) to obtain compound 6-9 (150 mg). ESI-MS m/z: 263.1 [M+H]⁺.

Compound 6-10 was prepared from 6-9 in 4 steps following the procedure described for synthesis of compound 5-12 from compound 5-8 in Example 5. ESI-MS m/z: 382.1 [M+H]⁺.

To a solution of compound 6-10 (36 mg, 0.095 mmol, 1.0 eq.) in DCM (1 mL) was added BBr₃ (1 mL) dropwise at −78° C. and the reaction mixture was stirred at rt under N₂ for overnight. The reaction mixture was quenched with MeOH (2 mL) and neutralized with NH₃/H₂O to pH 8-9 at −78° C. The mixture was concentrated under reduced pressure. The residue was dissolved in DMF (3 mL), filtered, the filtrate was purified by Prep-HPLC to afford Compound 119 (4.25 mg). ESI-MS m/z: 368.0 [M+H]⁺; 1H NMR (400 MHz, CD₃OD) δ: 7.35 (s, 1H), 6.73 (s, 1H), 4.44 (s, 2H), 4.32 (s, 2H), 3.56-3.48 (m, 411), 1.62-1.44 (m, 311), 0.95 (d, J=6.5 Hz, 6H).

### Example 1.7: Synthesis of 5-(7-hydroxy-3-(pyridin-3-yl)quinolin-6-yl)isothiazol-3(2H)-one 1,1-dioxide (Compound 115)

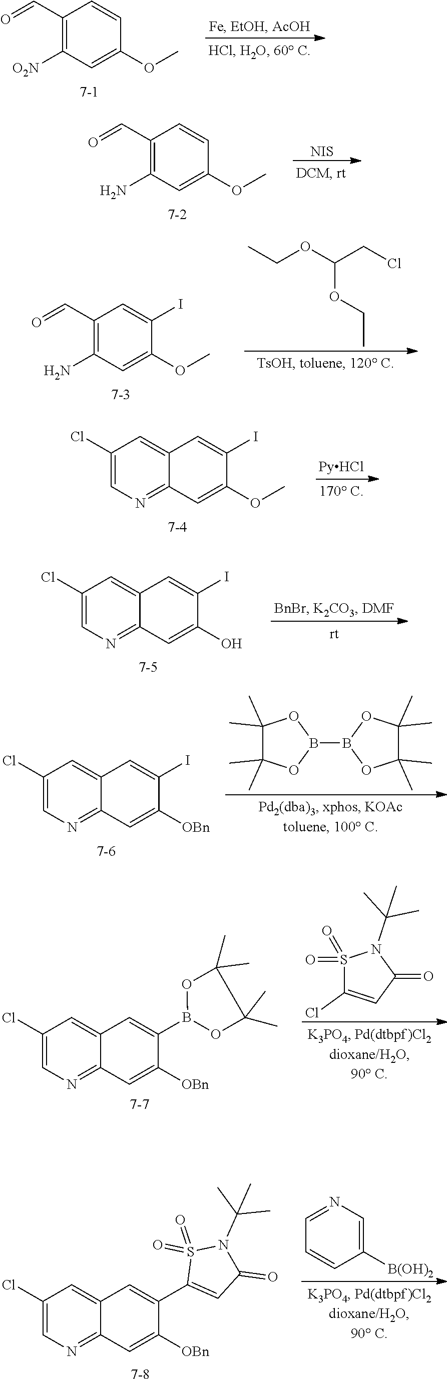

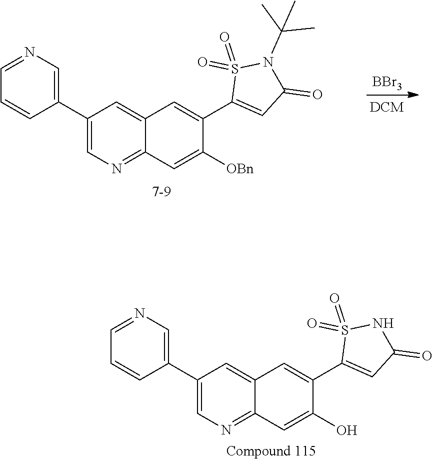

To a mixture of compound 7-1 (15 g, 86 mmol, 1.0 eq.) and Fe powder (26.8 g, 497 mmol, 6.0 eq.), EtOH (150 mL), AcOH (150 mL), H₂O (75 mL) and HCl (13 mL, 1.8 eq) were added. The reaction mixture was stirred at 60° C. under N₂ for 1 h. The reaction mixture was filtered and concentrated under reduced pressure. The pH value of the residue was adjusted to 7-8 with Na₂CO₃ at 0° C. The solution mixture was extracted with 3×500 mL of EA. The organic layers were combined, washed with brine, dried over Na₂SO₄ and concentrated under vacuum. The residue was purified by column chromatography on silica gel eluting with petroleum ether/ethyl acetate (2:1) to obtain compound 7-2 (11 g). ESI-MS m/z: 152 [M+H]⁺.

To a mixture of compound 7-2 (11 g, 72.36 mmol, 1.0 eq.) in DCM (150 mL) and NIS (17.9 g, 79.6 mmol, 1.1 eq.) was added. The mixture was stirred at rt for 3 h under N₂. The reaction mixture was diluted with PE (100 mL) and filtered to obtain compound 7-3 (20 g). ESI-MS m/z: 277.9 [M+H]⁺.

To a mixture of compound 7-3 (20 g, 72.2 mmol, 1.0 eq.) in toluene (300 mL) and 2-chloro-1,1-diethoxyethane (13.22 g, 86.62 mmol, 1.2 eq.), TsOH (2.74 g, 14.44 mmol, 0.2eq.) were added. The reaction mixture was stirred at 120° C. under N₂ for 4 h. The reaction mixture was diluted with DCM (500 mL) and washed with H₂O (2×100 mL), dried over anhydrous Na₂SO₄, filtered and concentrated under reduced pressure. The residue was purified by column chromatography on silica gel eluting with petroleum ether/ethyl acetate (1:1) to obtain compound 7-4 (10 g). ESI-MS m/z: 319.9 [M+H]⁺.

To mixture of compound 7-4 (10 g, 0.47 mmol) and pyridine hydrochloride (25 g) was stirred at 170° C. for 4 hr, cooled to r.t., quenched with water (500 mL) and extracted with DCM (1 L). The organic layer was separated, dried over anhydrous Na₂SO₄, and concentrated under reduced pressure. The residue was purified by flash column chromatography with (PE: EA=1:1) to afford compound 7-5 (3.8 g). ESI-MS m/z: 305.9 [M+H]⁺.

To mixture of compound 7-5 (3.8 g, 12.46 mmol) in DMF (50 mL) and K₂CO₃ (5.16 g, 37.37 mmol, 3 eq.), BnBr (3.19 g, 18.69 mmol, 1.5eq.) were added. The reaction was stirred at rt for 6 hr, quenched with water (500 mL) and extracted with EA (500 mL). The organic layer was separated, dried over anhydrous Na₂SO₄, and concentrated under reduced pressure. The residue was purified by flash column chromatography (PE:EA=3:1) to afford compound 7-6 (4 g). ESI-MS m/z: 395.8 [M+H]⁺.

To mixture of compound 7-6 (4 g, 10.12 mmol) in toluene (500 mL) and KOAc (2.48 g, 25.31 mmol, 2.5 eq.), X-Phos (482.7 mg, 1.01 mmol, 0.1 eq.), 4,4,4′,4′,5,5,5′,5′-octamethyl-2,2′-bi(1,3,2-dioxaborolane (5.14 g, 20.25 mmol, 2.0 eq), Pd₂dba₃ (927.3 mg, 1.01 mmol, 0.1 eq.) were added. The reaction was stirred 100° C. for overnight, quenched with water (200 mL) and extracted with EA (200 mL). The organic layer was separated, dried over anhydrous Na₂SO₄, and concentrated under reduced pressure. The residue was purified by flash column chromatography (PE:EA=3:1) to afford compound 7-7 (600 mg). ESI-MS m/z: 396 [M+H]⁺.

To mixture of compound 7-7 (600 mg, 1.52 mmol) in dioxane (2 mL) and K₃PO₄ (967.3 mg, 4.56 mmol, 3eq.), H₂O (0.5 ml), Pd(dtbpf)Cl₂ (99 mg, 0.152 mmol, 0.1 eq.) were added. The reaction was stirred at 90° C. for 3 hr, quenched with water (500 mL) and extracted with EA (500 mL). The organic layer was separated, dried over anhydrous Na₂SO₄, and concentrated under reduced pressure. The residue was purified by flash column chromatography (DCM:MeOH=10:1) to afford compound 7-8 (300 mg). ESI-MS m/z: 456.9 [M+H]⁺;

To mixture of compound 7-8 (300 mg, 0.65 mmol) in dioxane (2 mL) and K₃PO₄ (418.9 mg, 1.97 mmol, 3eq.), pyridin-3-ylboronic acid (161.5 mg, 1.31 mmol, 2eq.), H₂O (0.5 mL) and Pd(dtbpf)Cl₂ (64.31 mg, 0.098 mmol, 0.1 eq.) were added. The reaction was stirred at 90° C. for 3 hr, quenched with water (50 mL) and extracted with EA (50 mL). The organic layer was separated, dried over anhydrous Na₂SO₄, and concentrated under reduced pressure. The residue was purified by flash column chromatography (DCM:MeOH=10:1) to afford compound 7-9 (30 mg). ESI-MS m/z: 500 [M+H]⁺.

To mixture of compound 7-9 (30 mg, 0.65 mmol) in DCM (2 mL) and BBr₃ (2 mL, 1 mol/L) was added at −78° C. The reaction was stirred at −78° C. for 3 hr, quenched with MeOH (5 mL) and extracted with EA (30 mL). The organic layer was separated, dried over anhydrous Na₂SO₄, and concentrated under reduced pressure. The residue was purified by prep-HPLC to afford Compound 115 (1.8 mg). ESI-MS m/z: 354 [M+H]⁺; ¹H NMR (400 MHz, DMSO-d6): δ 11.41 (s, 1H), 9.25 (d, J=2.3 Hz, 1H), 9.15 (d, J=1.8 Hz, 1H), 8.83 (s, 1H), 8.69-8.58 (m, 2H), 8.40 (d, J=8.3 Hz, 1H), 7.60 (dd, J=7.9, 4.8 Hz, 1H), 7.48-7.41 (m, 1H), 7.20 (s, 1H), 6.95 (s, 1H).

### Example 1.8: Synthesis of methyl 5-(1,1-dioxido-4-oxo-1,2,5-thiadiazolidin-2-yl)-6-methoxybenzo[b]thiophene-2-carboxylate (Compound 102)

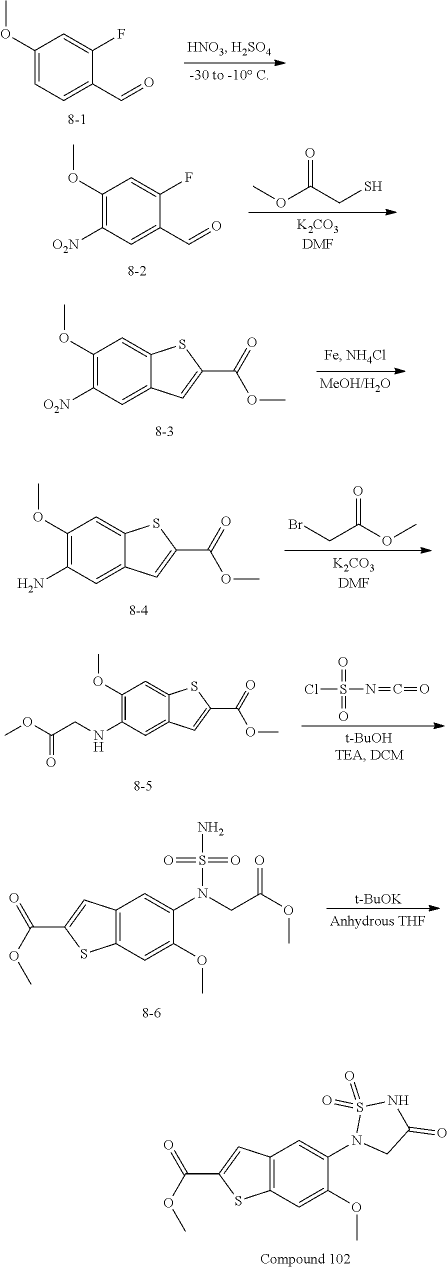

To a solution of 2-fluoro-4-methoxybenzaldehyde (8-1) (5 g, 32.46 mmol) in conc. H₂SO₄(100 mL) at −20° C., was added conc. HNO₃ (2.2 mL, 32.46 mmol in 5 mL of conc. H₂SO₄ slowly. The resulting mixture was stirred at −10° C. for 2 h. The reaction mixture was poured into ice-water and filtered. The solid collected was purified by flash chromatography on silica gel (20% EA/PE) to afford compound 8-2 (5.3 g).

To a solution of compound 8-2 (5.3 g, 26.6 mmol) in DMF (100 mL) at 0° C., was added K₂CO₃(7.3 g, 53.2 mmol) followed by methyl 2-mercaptoacetate (4.2 g, 39.9 mmol). The resulting mixture was heated to 80° C. and stirred for 16 h. The reaction mixture was extracted with EA, washed with water and brine, dried over Na₂SO₄, and concentrated in vacuo. The residue was purified by flash chromatography on silica gel (50% EA/PE) to afford compound 8-3 (2.2 g).

To a solution of compound 8-3 (2.2 g, 8.2 mmol) in EtOH/H₂O (60 mL/20 mL) was added NH₄Cl (1.7 g, 32.8 mmol) followed by Iron powder (918 mg, 16.4 mmol) slowly, and the resulting mixture was heated to 80° C. and stirred for 3 h. The reaction mixture was extracted with EA, washed with water and brine, dried over Na₂SO₄, and concentrated in vacuo. The residue was purified by flash chromatography on silica gel (50% PE/EA) to afford compound 8-4 (1.7 g). ESI-MS m/z: 238 [M+H]⁺.

To a solution of compound 8-4 (200 mg, 0.84 mmol) in DMF (10 mL) at 0° C., was added K₂CO₃ (347 mg, 2.52 mmol) followed by methyl 2-bromoacetate (253 mg, 1.68 mmol), and the resulting mixture was heated to 80° C. and stirred for 2 h. The reaction mixture was extracted with EA, washed with water and brine, dried over Na₂SO₄, and concentrated in vacuo. The residue was purified by flash chromatography on silica gel (40% EA/PE) to afford compound 8-5 (155 mg).

To a solution of sulfurisocyanatidic chloride (34 mg, 0.24 mmol) in DCM (20 mL) at 0° C., was added t-BuOH (18 mg, 0.24 mmol). After stirring at 0° C. for 30 min, a preformed solution of compound 8-5 (50 mg, 0.16 mmol) and TEA (32 mg, 0.32 mmol) in DCM was added slowly at 0° C. The resulting mixture was stirred at 0° C. for 30 min and then was allowed to warm to room temperature and stirred for 1 h. The reaction mixture was quenched with water, extracted with DCM, washed with brine, dried over Na₂SO₄, and concentrated in vacuo. The residue was purified by flash chromatography on silica gel (50% EA/PE) to afford compound 8-6 (28 mg).

To a solution of compound 8-6 (28 mg, 0.07 mmol) in anhydrous tetrahydrofuran (10 mL), was added t-BuOK (12 mg, 0.11 mmol) under argon. The resulting mixture was stirred at rt for 5 h. The reaction mixture was acidified (pH=1) with 3 M HCl, extracted with EA, washed with water and brine, dried over Na₂SO₄, and concentrated in vacuo. The residue was purified by flash chromatography on silica gel (10% MeOH/DCM) to afford Compound 102 (10.1 mg). ESI-MS m/z: 355 [M−H]⁻. ¹H NMR (400 MHz, DMSO-d6): δ 8.14 (s, 1H), 8.10 (s, 1H), 7.70 (s, 1H), 4.06 (s, 2H), 3.88 (s, 3H), 3.86 (s, 3H).

### Example 1.9: Synthesis of 5-(7-hydroxy-3-(isopentylamino)quinolin-6-yl)-1,2,5-thiadiazolidin-3-one 1,1-dioxide (142)

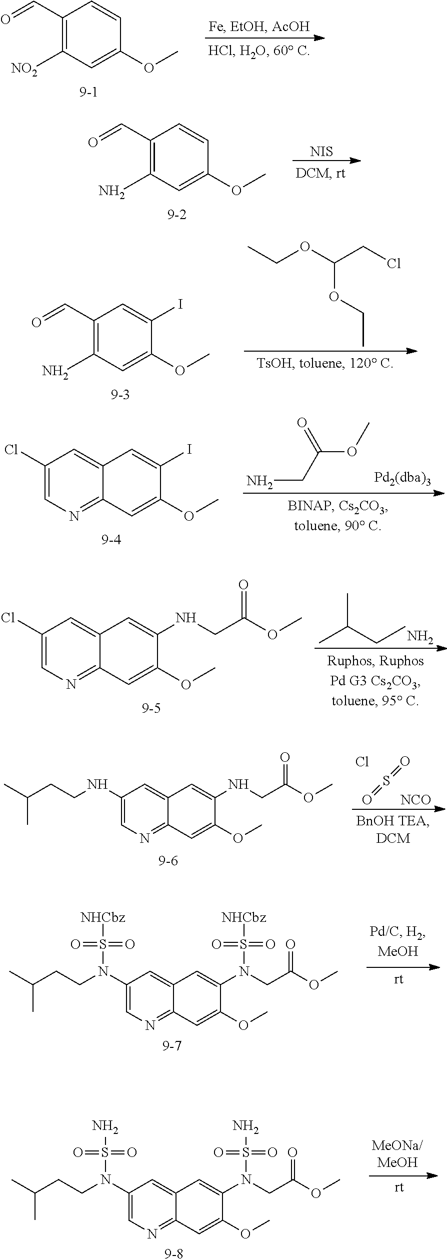

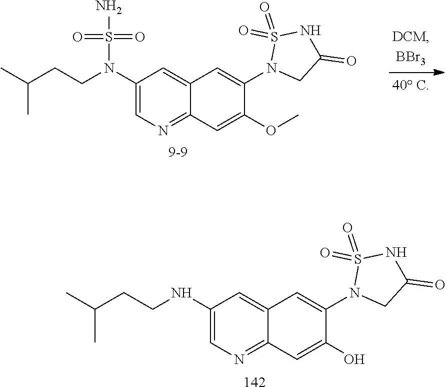

To a mixture of compound 9-1 (15 g, 86 mmol, 1.0 eq.) in EtOH (150 mL), AcOH (150 mL) and H₂O (75 mL) was added Fe powder (26.8 g, 497 mmol, 6.0 eq.) and HCl(13 mL, 1.8 eq). The reaction mixture was stirred at 60° C. under N₂ for 1 h. The reaction mixture was filtered and the filtrate was concentrated under reduced pressure. The pH value of the residue was adjusted to 7-8 with sat. Na₂CO₃ solution at 0° C. The solution mixture was extracted with EA (3×200 mL). The combined organic layers were washed with brine (3×500 mL), dried over Na₂SO₄ and concentrated under vacuum. The residue was purified by column chromatography on silica gel eluting with petroleum ether/ethyl acetate (2:1) to obtain compound 9-2 (11 g). ESI-MS m/z: 152 [M+H]⁺.

To a mixture of compound 9-2 (11 g, 72.36 mmol, 1.0 eq.) in DCM (150 mL) was added NIS (17.9 g, 79.6 mmol, 1.1 eq.). The mixture was stirred at rt for 3 h under N₂. The reaction mixture was diluted with PE (100 mL) and filtered. The solid was collected and dried under reduced pressure to obtain compound 9-3 (20 g). ESI-MS m/z: 277.9 [M+H]⁺.

To a mixture of compound 9-3 (20 g, 72.2 mmol, 1.0 eq.) in toluene (300 mL) was added 2-chloro-1,1-diethoxyethane (13.22 g, 86.62 mmol, 1.2 eq.) and TsOH (2.74 g, 14.44 mmol, 0.2eq.). The reaction mixture was stirred at 120° C. under N₂ for 4 h. The reaction mixture was diluted with DCM (500 mL) and washed with H₂O (2×200 mL), dried over anhydrous Na₂SO₄, filtered and concentrated under reduced pressure. The residue was purified by column chromatography on silica gel eluting with petroleum ether/ethyl acetate (1:1) to obtain compound 9-4 (10 g). ESI-MS m/z: 319.9 [M+H]⁺.

To mixture of compound 9-4 (4.6 g, 14.42 mmol, 1.0 eq.) in toluene (100 mL) was added BINAP (1.8 g, 2.884 mmol, 0.2 eq.), Glycine methyl ester hydrochloride (2.17 g, 17.3 mmol, 1.2 eq.), Cs₂CO₃(14.1 g, 3 eq, 43.26 mmmol) and Pd₂(dba)₃ (1.21 g, 1.442 mmol, 0.1 eq). The reaction was stirred at 90° C. for overnight under N₂. The reaction mixture filtered and the filtrate was concentrated under reduced pressure. The residue was purified by column chromatography on silica gel eluting with petroleum ether/ethyl acetate (1:1) to obtain compound 9-5 (2.2 g). ESI-MS m/z: 281 [M+H]⁺.

To mixture of compound 9-5 (970 mg, 3.46 mmol) in toluene (15 mL) was added Isoamylamine(447.4 mg, 5.2 mmol, 1.5 eq), Cs₂CO₃ (5.65 g, 17.32 mmol, 5eq.), Ruphos 162 mg, 0.346 mmol, 0.1eq.) and Ruphos Pd G3 (290 mg, 0.346 mmol, 0.1eq.). The reaction was stirred at 95° C. for overnight under N₂. The reaction mixture filtered and the filtrate was concentrated under reduced pressure. The residue was purified by column chromatography on silica gel eluting with petroleum ether/ethyl acetate (1:1) to obtain compound 9-6 (510 mg). ESI-MS m/z: 332.2 [M+H]⁺.

To a mixture of sulfurisocyanatidic chloride (864 mg, 6.1 mmol, 10 eq) in DCM (5 mL) was added BuOH (660 mg, 6.1 mmol) at 0° C. The reaction was stirred at rt for Ih. Then a solution of compound 9-6 (250 mg, 0.61 mmol, 1 eq) and TEA(1.23 g, 12.2 mmol, 20 eq) in DCM (5 mL) was added to the mixture. The reaction was stirred at 0° C. for overnight. The mixture was quenched with water (30 mL) and extracted with DCM (30 mL). The organic layer was separated, dried over anhydrous Na₂SO₄, and concentrated under reduced pressure. The residue was purified by flash column chromatography (MeOH:DCM=3:1) to afford compound 9-7 (400 mg). ESI-MS m/z: 758 [M+H]⁺.

To mixture of compound 9-7 (400 mg, 0.528 mmol, 1 eq) in MeOH (5 ml) was Pd/C (40 mg, 10% wt). The reaction was stirred at rt for 6 hr under H₂ (30 psi). The reaction mixture was filtered and the filtrate was concentrated under reduced pressure to afford compound 9-8 (100 mg). ESI-MS m/z: 490.2 [M+H]⁺.

To a mixture of compound 9-8 (100 mg, 0.205 mmol) and 4A MS (100 mg) in THF (2 mL) and MeOH (0.5 mL) was added MeONa (0.3 mL, 1.62 mmol, 5.4 M) under N₂. The reaction was stirred at rt for 2 hr. The reaction mixture concentrated under reduced pressure. The residue was purified by flash column chromatography (EA: MeOH=10:1) to afford compound 9-9 (30 mg). ESI-MS m/z: 456.1[M−H]⁻.

To mixture of compound 9-9 (30 mg, 0.658 mmol) in DCM (2 mL) was added BBr₃ (2 ml, 1 mol/L) at −78° C. The reaction was stirred at 40° C. for 16 h under N₂. The mixture was quenched with MeOH (5 mL) and adjusted to pH=8-9 with NH₃·H₂O at −78° C. The mixture was concentrated under reduced pressure. The residue was purified by prep-HPLC separation to afford 142 (1.0 mg). ESI-MS m/z: 363.1 [M−H]⁻; ¹H NMR (400 MHz, CD₃OD): δ 8.29 (d, J=2.4 Hz, 1H), 7.84 (s, 1H), 7.24 (s, 1H), 7.09 (d, J=2.4 Hz, 1H), 4.49 (s, 2H), 3.18-3.15 (m, 2H), 1.81-1.76 (m, 1H), 1.61-1.55 (m, 2H), 0.99 (d, J=6.6 Hz, 6H).

### Example 1.10: Synthesis of 5-(8-fluoro-6-hydroxy-2-(isopentyloxy)quinolin-7-yl)-1,2,5-thiadiazolidin-3-one 1,1-dioxide (135)

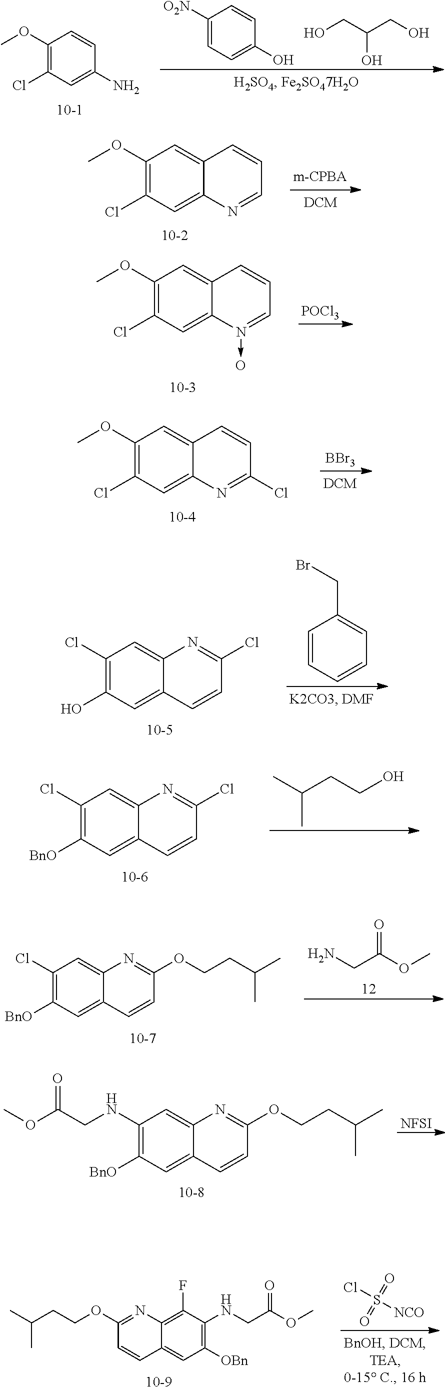

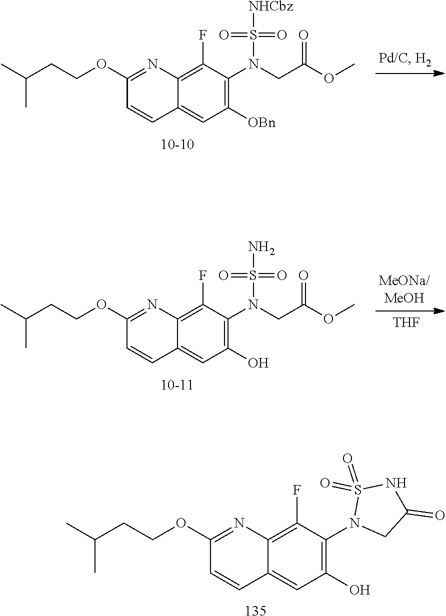

A mixture of compound 10-1 (20 g, 126.9 mmol, 1 eq.), 4-nitrophenol (19.4 g, 139.6 mmol, 1.1 eq.), Fe₂SO₄7H₂O (1.8 g, 6.35 mmol, 0.05 eq.) and propane-1,2,3-triol (56 ml) in H₂SO₄ (25 ml) was stirred at 140° C. under N₂ for 16 h. The reaction mixture was poured into ice water and diluted with DCM (2 L). After partition, the organic layer was washed with H₂O (4 L), dried over anhydrous Na₂SO₄, filtered and concentrated under reduced pressure. The residue was purified by column chromatography on silica gel eluting with dichloromethane/ethyl acetate (1/0-1/10) to obtain compound 10-2 (20 g). ESI-MS m/z: 193.9 [M+H]⁺.

To a mixture of compound 10-2 (20 g, 103.3 mmol, 1.0 eq.) in DCM (100 ml) was added m-CPBA (24.96 g, 144.6 mmol, 1.4 eq.). The mixture was stirred at room temperature for 16 h under N₂. The reaction mixture was adjusted pH to 9-10 by 1 M NaOH solution and then diluted with DCM (500 ml). After partition, the organic layer was washed with H₂O (3×200 mL), dried over anhydrous Na₂SO₄, filtered and concentrated under reduced pressure. The residue was purified by column chromatography on silica gel eluting with eluting with dichloromethane/menthol (1/0-10:1, v/v) to obtain compound 10-3 (20 g). ESI-MS m/z: 210.1 [M+H]⁻.

A mixture of compound 10-3 (20 g, 95.4 mmol, 1.0 eq.) in POCl₃ (60 ml) was stirred at 100° C. under N₂ for 16 h. The reaction mixture was concentrated under reduced pressure. The residue was purified by column chromatography on silica gel eluting with petroleum ether/ethyl acetate (1/0-12/1) to obtain compound 10-4 (10 g). ESI-MS m/z: 228.0 [M+H]⁻.

To a mixture of 10-4 (2.7 g, 11.9 mmol, 1.0 eq.) was added dropwise BBr3 (6 g, 23.8 mmol, 2.0 eq.) at −78° C. under Ar. After addition, the mixture was stirred at room temperature for 16 h. The reaction mixture was quenched by NH3·H2O and diluted with DCM (500 ml). After partition, the organic layer was dried over anhydrous Na₂SO₄, filtered and concentrated under reduced pressure. The residue was purified by column chromatography on silica gel eluting with petroleum ether/ethyl acetate (1/0-4/1) to obtain compound 10-5 (1.5 g). ESI-MS m/z: 214.1 [M+H]⁺.

A mixture of compound 10-5 (1.5 g, 7.01 mmol, 1.0 eq.), compound K2CO3 (2.9 g, 21.03 mmol, 3.0 eq.) and (bromomethyl)benzene (1.32 g, 7.71 mmol, 1.1 eq.) in DMF (20 ml) was stirred at room temperature for 16 h under N₂. The reaction mixture was diluted with DCM (100 ml) and washed with H₂O (3×40 mL). The organic layer was dried over anhydrous Na₂SO₄, filtered and concentrated under reduced pressure. The residue was purified by column chromatography on silica gel eluting with eluting with dichloromethane/menthol (1/0-10:1, v/v) to obtain compound 10-6 (1.5 g). ESI-MS m/z: 304.0 [M+H]⁺.

To a mixture of 3-methylbutan-1-ol (730 mg, 8.3 mmol, 2.0 eq.) in DMF (5 mL) was added NaH (300 mg, 12.45 mmol, 3.0 eq., 40% in mineral oil) at 0° C. under Ar. The mixture was stirred at 0° C. for 0.5 h. Then a solution of compound 10-6 (1 g, 4.15 mmol, 1.0 eq.) in DMF (1 mL) was added dropwise to the mixture at 0° C. under Ar. After addition, the mixture was stirred at room temperature for 16 h. The reaction mixture was quenched by NH₄Cl and diluted with DCM (50 mL). After partition, the organic layer was ried over anhydrous Na₂SO₄, filtered and concentrated under reduced pressure. The residue was purified by column chromatography on silica gel eluting with petroleum ether/ethyl acetate (1/0-10/1) to obtain compound 10-7 (1.06 g). ESI-MS m/z: 356.1 [M+H]⁺.

A mixture of compound 10-7 (1.06 g, 3.09 mmol, 1.0 eq.), methyl glycinate (585 mg, 4.64 mmol, 1.5 eq.), RuphosPdG3 (388 mg, 0.464 mmol, 0.15 eq.), Ruphos (216 mg, 0.464 mmol, 0.15 eq.) and Cs2CO3 (4.04 g, 12.4 mmol, 4.0 eq.) in dioxane (30 mL) was stirred at 90° C. under Ar for 16 h. The reaction mixture was filtered and concentrated under reduced pressure. The residue was purified by column chromatography on silica gel eluting with dichloromethane/ethyl acetate (1/0-1/10) to obtain compound 10-8 (800 mg). ESI-MS m/z: 409.2 [M+H]⁺.

To a mixture of compound 10-8 (500 mg, 1.226 mmol, 1.0 eq.) in DMF (10 ml) was added NFSI (576 mg, 1.83 mmol, 1.5 eq.) at 0° C. under Ar. After addition, the mixture was stirred at room temperature for 16 h. The reaction mixture was quenched by NH₄Cl and diluted with DCM (50 ml). After partition, the organic layer was ried over anhydrous Na₂SO₄, filtered and concentrated under reduced pressure. The residue was purified by C-18 Flash with water/acetonitrile (1/0-1/1) to obtain compound 10-9 (130 mg). ESI-MS m/z: 427.0 [M+H]⁺.

To a mixture of sulfurisocyanatidic chloride (433.1 mg, 3.05 mmol, 10.0 eq.) in DCM (10 mL) was added BnOH (329.4 mg, 3.05 mmol, 10.0 eq.) at 0° C. under N₂. The reaction mixture was stirred at 0° C. under N₂ for 0.5 h. Then a solution of compound 10-9 (130 mg, 0.305 mmol, 1.0 eq.) and TEA (616.1 mg, 6.1 mmol, 20.0 eq.) in DCM (6 mL) was added to the mixture at 0° C. under N₂. The reaction mixture was stirred at room temperature for 16 h under N₂. The reaction mixture was quenched with H₂O (30 mL) and extracted with with DCM (3×30 mL). The combined organic layers were dried over anhydrous Na₂SO₄, filtered and concentrated under reduced pressure to obtain crude compound 10-10 (180 mg), which was used for the next step without further purification. ESI-MS m/z: 640.0 [M+H]⁺.

To a mixture of compound 10-10 (160 mg, 0.25 mmol, 1.0 eq.) in MeOH (10 ml) was added Pd/C (160 mg, 10% wt). The mixture was stirred at room temperature under H₂ (30 psi) for 2 h. The reaction mixture was filtered. The filtrate was concentrated under reduced pressure to obtain compound 10-11 (60 mg), which was used for the next step without further purification. ESI-MS m/z: 416.0 [M+H]⁺.

To a mixture of compound 10-11 (60 mg, 0.144 mmol, 1.0 eq.) and 4A MS (100 mg) in THF (5 mL) and MeOH (1 mL) was added NaOMe solution (1.44 ml, 1.44 mmol, 10 eq., 5.4 M in MeOH) under N₂. After addition, the mixture was stirred at room temperature for 1 h under N₂. The reaction mixture was concentrated under reduced pressure. The residue was purified by HPLC with (0.1% NH₃H₂O) to obtain compound 135 (8.11 mg). ESI-MS m/z: 382.1 [M−H]⁻; ¹H NMR (400 MHz, CD₃OD): δ 7.94 (d, J=8.8 Hz, 1H), 7.00 (s, 1H), 6.91 (d, J=8.8 Hz, 1H), 4.50-4.44 (m, 2H), 4.42 (s, 2H), 1.88-1.78 (m, 1H), 1.76-1.66 (m, 2H), 0.99 (d, J=8.0 Hz, 6H).

### Example 1.11: Synthesis of 5-(8-fluoro-2-(4-fluorophenethoxy)-6-hydroxyquinolin-7-yl)-1,2,5-thiadiazolidin-3-one 1,1-dioxide (204)

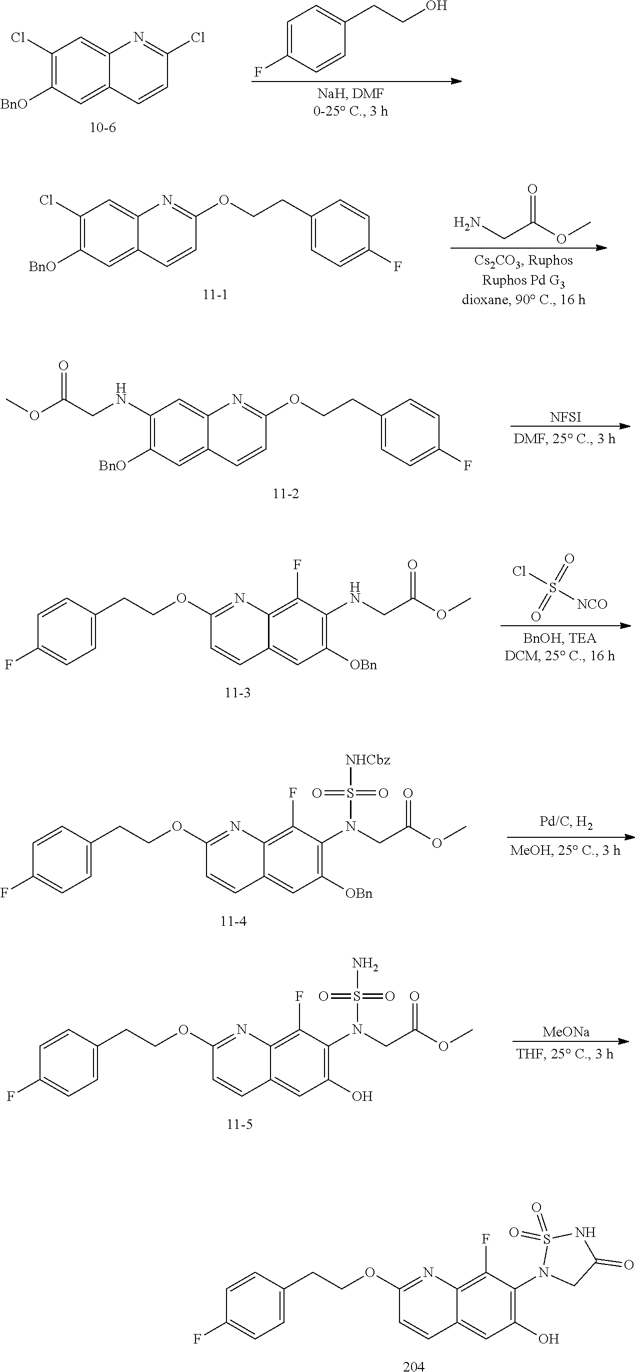

To a mixture of 2-(4-fluorophenyl)ethan-1-ol (1.39 g, 9.9 mmol, 1.5 eq.) in DMF (20 mL) was added NaH (396 mg, 9.9 mmol, 1.5 eq.) at 0° C. The mixture was stirred at 0° C. under N₂ for 0.5 h. Then compound 10-6 (2.0 g, 6.6 mmol, 1 eq.) in DMF (5 mL) was added to the mixture. The reaction mixture was stirred at 25° C. under N₂ for 3 h. The reaction mixture was diluted with DCM (50 mL) and washed with H₂O (2×50 mL), dried over anhydrous Na₂SO₄, filtered and concentrated under reduced pressure. The residue was purified by column chromatography on silica gel eluting with petroleum ether/ethyl acetate (1/0-5/1) to give compound 11-1 (1.2 g).

To a mixture of compound 11-1 (1.2 g, 2.95 mmol, 1.0 eq.), methyl glycinate (555 mg, 4.42 mmol, 1.5 eq.), Cs₂CO₃ (3.8 g, 11.8 mmol, 4.0 eq.) and Ruphos (685 mg, 1.47 mmol, 0.5 eq.) in dioxane (30 mL) was added Ruphos Pd G₃ (494 mg, 0.59 mmol, 0.2 eq.) under N₂. The reaction mixture was stirred at 90° C. under N₂ for 16 h. The reaction mixture was diluted with DCM (30 mL) and washed with H₂O (2×30 mL), dried over anhydrous Na₂SO₄, filtered and concentrated under reduced pressure. The residue was purified by column chromatography on silica gel eluting with petroleum ether/ethyl acetate (1/0-3/1) to obtain compound 11-2 (700 mg, 51.6%) as a yellow oil. ESI-MS m/z: 461.1 [M+H]⁺.

To a mixture of compound 11-2 (600 mg, 1.3 mmol, 1.0 eq.) in DMF (20 mL) was added NFSI (534 mg, 1.7 mmol, 1.3 eq.) at 0° C. The reaction mixture was stirred at 25° C. under N₂ for 3 h. LCMS and TLC (petroleum ether/ethyl acetate=3/1) showed the desired product was formed. The reaction mixture was diluted with DCM (20 mL) and washed with H₂O (2×20 mL), dried over anhydrous Na₂SO₄, filtered and concentrated under reduced pressure. The residue was purified by prep-HPLC (FA) to obtained compound 11-3 (200 mg). ESI-MS m/z: 479.2 [M+H]⁺.

To a mixture of sulfurisocyanatidic chloride (603 mg, 4.18 mmol, 10.0 eq.) in DCM (10 mL) was added BnOH (444 mg, 4.18 mmol, 10.0 eq.). The reaction mixture was stirred at room temperature under N₂ for 1 h. Then compound 11-3 (200 mg, 0.418 mmol, 1.0 eq.) and TEA (844 mg, 8.36 mmol, 20 eq.) in DCM (3 mL) was added to the mixture. The reaction mixture was stirred at room temperature for 16 h under N₂. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-1/1) to obtain compound 11-4 (110 mg). ESI-MS m/z: 692.0 [M+H]⁺.

To a mixture of compound 11-4 (110 mg, 0.16 mmol, 1.0 eq.) in MeOH (5 mL) was added Pd/C (30 mg, 10%, 0.2 eq.). The reaction mixture was stirred at room temperature under H₂ (30 psi) for 2 h. LCMS showed compound 5 was consumed and the desired product was formed. The reaction mixture was filtered and concentrated under reduced pressure to obtain compound 11-5 (50 mg), which was used in the next step without further purification. ESI-MS m/z: 467.9 [M+H]⁺.

To a mixture of compound 11-5 (50 mg, 0.107 mmol, 1.0 eq.) in THF (5 mL) was added NaOMe (0.9 mL, 0.535 mmol, 5.0 eq.) under N₂. After addition, the mixture was stirred at room temperature for 2 h under N₂. The reaction mixture was filtered and concentrated to give crude product, which was purified by prep-HPLC (NH₃·H₂O) to obtained compound 204 (1.68 mg). ESI-MS m/z: 434.1 [M−H]⁻; ¹H NMR (400 MHz, CD₃OD) δ 7.94 (dd, J=9.2, 1.2 Hz, 1H), 7.33 (dd, J=8.8, 5.6 Hz, 2H), 7.03-6.98 (m, 311), 6.88 (d, J=8.8 Hz, 1H), 4.62 (t, J=8.0 Hz, 2H), 4.40 (s, 2H), 3.10 (t, J=6.8 Hz, 2H).

### Example 1.12: Synthesis of 5-(1-fluoro-3-hydroxy-7-((tetrahydro-2H-pyran-4-yl)methoxy)naphthalen-2-yl)-1,2,5-thiadiazolidin-3-one 1,1-dioxide (200)

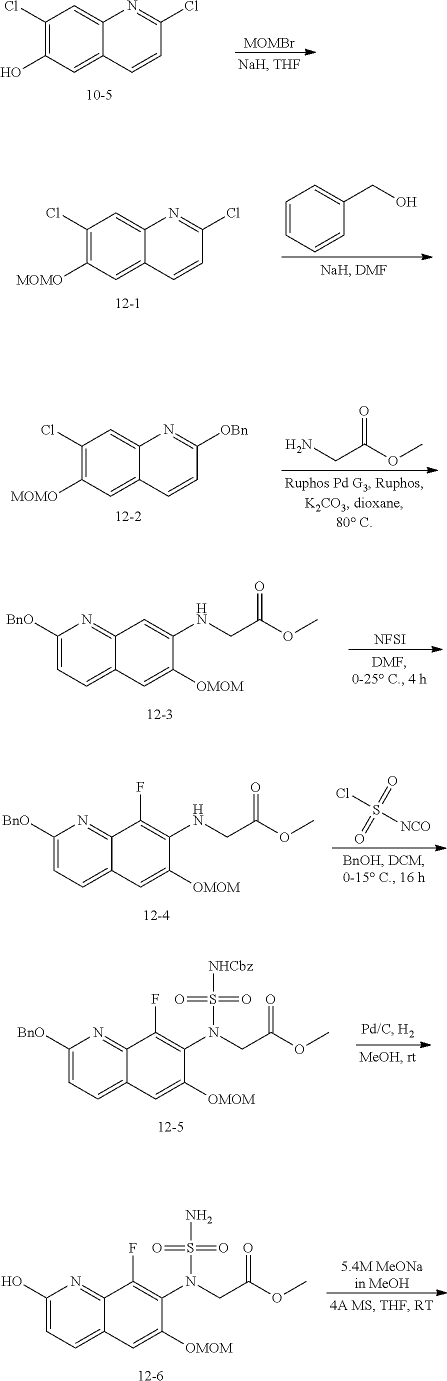

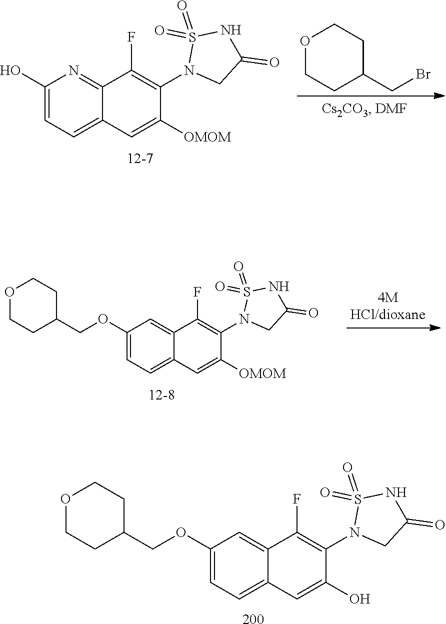

To a solution of compound 10-5 (1.2 g, 5.63 mmol, 1.0 eq.) and NaH (270 mg, 6.75 mmol, 1.2 eq.) in THF (20 ml) was added MOMBr (775 mg, 6.20 mmol, 1.1 eq.) dropwise at 0° C. The resulting mixture was stirred at rt for overnight under N₂. The reaction mixture was diluted with EA (50 mL) and washed with H₂O (2×50 mL), dried over anhydrous Na₂SO₄, filtered and concentrated under reduced pressure. The residue was purified by column chromatography on silica gel eluting with petroleum ether/ethyl acetate (1/0-5/1) to obtain compound 12-1 (1.4 g). ESI-MS m/z: 258 [M+H]⁺.

To a solution of BnOH (328 mg, 3.03 mmol, 1.2 eq.) and NaH (121 mg, 3.03 mmol, 1.2 eq.) in DMF (6 mL) was added compound 12-1 (650 mg, 2.53 mmol, 1.0 eq.). The reaction mixture was stirred at room temperature under 16 hours under N₂. The reaction mixture was diluted with EA (50 mL) and washed with H₂O (2×50 mL), dried over anhydrous Na₂SO₄, filtered and concentrated under reduced pressure. The residue was purified by column chromatography on silica gel eluting with PE/EA (1/0-5/1) to obtain compound 12-2 (750 mg). ESI-MS m/z: 330.0 [M+H]⁺.

The reaction mixture of compound 12-2 (3.2 g, 9.73 mmol, 1.0 eq.), Glycinemethylester hydrochloride (1.83 g, 14.59 mmol, 1.5 eq.), Ruphos Pd G₃ (2.44 g, 2.92 mmol, 0.3 eq.), Ruphos (2.26 g, 4.86 mmol, 0.5 eq.) and Cs₂CO₃ (12.65 g, 38.92 mmol, 4 eq.) in Dioxane (100 mL) was stirred at 80° C. under N₂ for 16 hours. The reaction mixture was concentrated under reduced pressure. The residue was purified by column chromatography on silica gel eluting with PE/EA (1/0-2/1) to give compound 12-3 (2.4 g). ESI-MS m/z: 383.1 [M+H]⁺.

To a mixture of compound 12-3 (2.4 g, 6.3 mmol, 1.0 eq.) in DMF (50 mL) was added NFSI (2.58 mg, 8.2 mmol, 1.3 eq.) at 0° C. The reaction mixture was stirred at 25° C. under N₂ for 3 h. The reaction mixture was diluted with DCM (30 mL) and washed with H₂O (2×30 mL), dried over anhydrous Na₂SO₄, filtered and concentrated under reduced pressure. The residue was purified by prep-HPLC (FA) to obtain compound 12-4 (800 mg). ESI-MS m/z: 401.0 [M+H]⁺.

To a solution of sulfurisocyanatidic chloride (2.89 g, 20.1 mmol, 10.0 eq.) in DCM (50 ml) was added BnOH (2.13 g, 20.1 mmol, 10.0 eq.) at 0° C. The resulting mixture was stirred at rt for 30 mins under N₂. Compound 12-4 (800 mg, 2.0 mmol, 1 eq.) and Et₃N (4.06 g, 40.2 mmol, 20 eq.) were dissolved in DCM (10 ml) and added dropwise to the mixture at 0° C. The reaction was warmed to rt for overnight. The reaction mixture was diluted with DCM (50 mL) and washed with H₂O (2×50 mL), dried over anhydrous Na₂SO₄, filtered and concentrated under reduced pressure. This to give compound 12-5 (1.1 g), which was used in the next step without further purification. ESI-MS m/z: 614.0 [M+H]⁺.

To a solution of compound 12-5 (1.1 g, 1.79 mmol, 1.0 eq.) in MeOH (20 mL) was added P/C (200 mg) under H₂ (30 psi). The reaction mixture was stirred at room temperature for 2 hours with an insert atmosphere of H₂. The reaction mixture was filtered, the filtrate was concentrated under reduced pressure to give compound 12-6 (600 mg), which was used in the next step without further purification. ESI-MS m/z: 389.9 [M+H]⁺.

To a mixture of compound 12-6 (600 mg, 1.54 mmol, 1.0 eq.) and 4A MS (500 mg) in THF (20 mL) was added Sodium methoxide (1.43 mL, 7.71 mmol, 5.0 eq.) dropwise. The reaction mixture was stirred at room temperature under N₂ for 16 h. The reaction mixture was concentrated under reduced pressure. The residue was purified by column chromatography on silica gel eluting with ethyl acetate/MeOH (1/0-4/1) to obtain compound 12-7 (450 mg). ESI-MS m/z: 356.0 [M+H]⁺.

To a mixture of compound 12-7 (50 mg, 0.14 mmol, 1.0 eq.) and Cs₂CO₃ (91 mg, 0.28 mmol, 2.0 eq.) in DMF (3 mL) was added 4-(bromomethyl)tetrahydro-2H-pyran (75 mg, 0.42 mmol, 3.0 eq.). The reaction mixture was stirred at 80° C. for 16 h. The reaction mixture was filtered, the filtrate was concentrated under reduced pressure to give crude compound 12-8 (40 mg). ESI-MS m/z: 454.3 [M−H]⁻.

A solution of compound 12-8 (40 mg, 0.09 mmol, 1.0 eq.) in HCl/dioxane (4M 2 mL) was stirred at rt for 3 hours. The mixture was concentrated under reduced pressure. The residue was dissolved in DMF (2 ml) and purified by Pre-HPLC (NH₃·H₂O) to obtain compound 200 (4.01 mg). ESI-MS m/z: 410.3 [M−H]⁻; ¹H NMR (400 MHz, CD₃OD): δ 7.95 (d, J=8.8 Hz, 1H), 7.01 (d, J=1.6 Hz, 11H), 6.92 (d, J=8.8 Hz, 11H), 4.41 (s, 2H), 4.31 (d, J=6.4 Hz, 2H), 3.98 (dd, J=11.2, 3.2 Hz, 2H), 3.47 (td, J=12.0, 1.6 Hz, 2H), 2.19-2.07 (m, 11H), 1.81-1.72 (m, 2H), 1.52-1.40 (m, 2H).

### Example 1.13: Synthesis of 5-(6-hydroxy-2-(isopentylamino)quinolin-7-yl)-4-methyl-1,2,5-thiadiazolidin-3-one 1,1-dioxide (137)

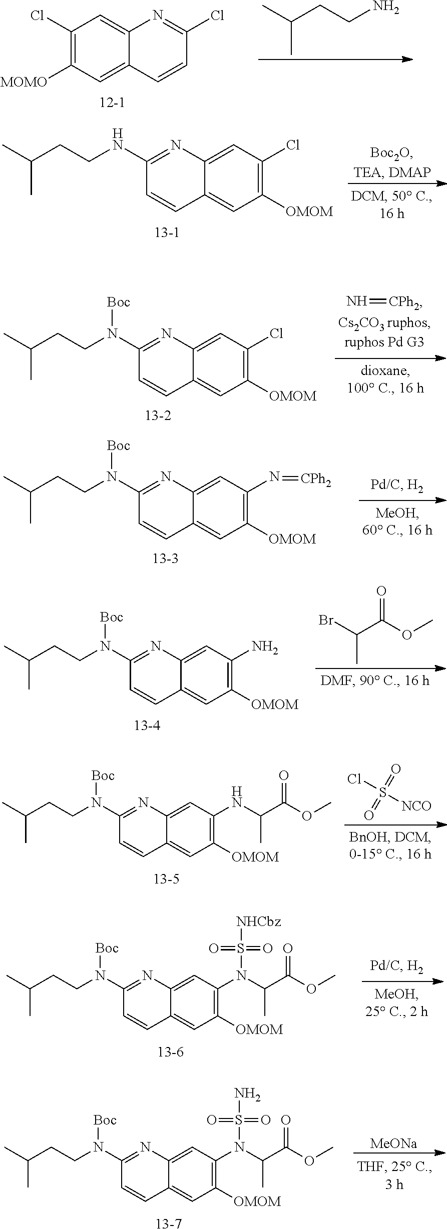

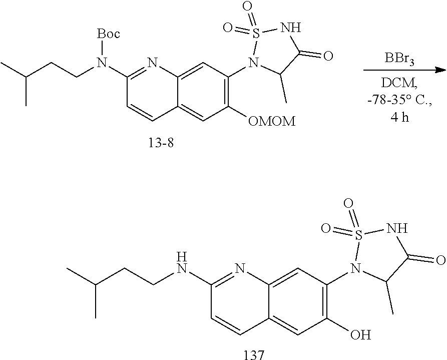

To a mixture of compound 12-1 (1.5 g, 5.84 mmol, 1 eq.) in 2-phenylethan-1-amine (15 mL) was added TEA (2.95 g, 29.18 mmol, 5 eq.). The reaction mixture was stirred at 120° C. under N₂ for 16 h in a sealed tube. The reaction mixture was concentrated under reduced pressure. The residue was purified by column chromatography on silica gel eluting with petroleum ether/ethyl acetate (1/0-4/1) to give compound 13-1 (1.2 g). ESI-MS m/z: 309.0 [M+H]⁺.

To a mixture of compound 13-1 (1.2 g, 3.89 mmol, 1.0 eq.), Boc₂O (1.27 g, 5.84 mmol, 1.5 eq.) and TEA (1.18 g, 11.67 mmol, 3.0 eq.) in THF (30 mL) was added DMAP (475 mg, 3.89 mmol, 1.0 eq.) under N₂. The reaction mixture was stirred at 70° C. under N₂ for 16 h. The reaction mixture was diluted with DCM (50 mL) and washed with H₂O (2×50 mL), dried over anhydrous Na₂SO₄, filtered and concentrated under reduced pressure. The residue was purified by column chromatography on silica gel eluting with petroleum ether/ethyl acetate (1/0-10/1) to obtain compound 13-2 (900 mg).

To a mixture of compound 13-2 (900 mg, 2.21 mmol, 1.0 eq.), diphenylmethanimine (599 mg, 3.31 mmol, 1.5 eq.), Cs₂CO₃ (2.87 g, 8.82 mmol, 4.0 eq.) and Ruphos (517 mg, 1.11 mmol, 0.5 eq.) in dioxane (15 mL) was added Ruphos Pd G₃ (552 mg, 0.66 mmol, 0.3 eq.) under N₂. The reaction mixture was stirred at 90° C. under N₂ for 16 h. The reaction mixture was diluted with DCM (30 mL) and washed with H₂O (2×30 mL), dried over anhydrous Na₂SO₄, filtered and concentrated under reduced pressure. The residue was purified by column chromatography on silica gel eluting with petroleum ether/ethyl acetate (1/0-2/1) to obtain compound 13-3 (700 mg). ESI-MS m/z: 554.1 [M+H]⁺.

To a mixture of compound 13-3 (700 mg, 1.27 mmol, 1.0 eq.) in MeOH (15 mL) was added Pd/C (150 mg, 10%, 0.1 eq.). The reaction mixture was stirred at room temperature under H₂ for 2 h. The reaction mixture was filtered and concentrated under reduced pressure to obtain compound 13-4 (350 mg), which was used in the next step without further purification. ESI-MS m/z: 390.1 [M+H]⁺.

To a mixture of compound 13-4 (350 mg, 0.9 mmol, 1.0 eq.) and K₂CO₃ (621 mg, 4.5 mmol, 5.0 eq.) in DMF (10 mL) was added methyl 2-bromopropanoate (297 mg, 1.8 mmol, 2 eq.) at 90° C. The reaction mixture was stirred at 90° C. under N₂ for 16 h. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-3/1) to obtain compound 13-5 (200 mg). ESI-MS m/z: 476.2 [M+H]⁺.

To a mixture of sulfurisocyanatidic chloride (606 mg, 4.2 mmol, 10.0 eq.) in DCM (15 mL) was added BnOH (446 mg, 4.2 mmol, 10.0 eq.). The reaction mixture was stirred at room temperature under N₂ for 1 h. Then compound 13-5 (200 mg, 0.42 mmol, 1.0 eq.) and TEA (850 mg, 8.4 mmol, 20.0 eq.) in DCM (3 mL) was added to the mixture. The reaction mixture was stirred at room temperature for 16 h under N₂. The reaction mixture was diluted with DCM (20 mL) and washed with H₂O (2×20 mL), dried over anhydrous Na₂SO₄, filtered and concentrated under reduced pressure. The residue was purified by column chromatography on silica gel eluting with petroleum ether/ethyl acetate (1/0-1/1) to obtain compound 13-6 (130 mg). ESI-MS m/z: 689.2[M+H]⁺.

To a mixture of compound 13-6 (130 mg, 0.19 mmol, 1.0 eq.) in MeOH (5 mL) was added Pd/C (30 mg, 10%, 0.1 eq.). The reaction mixture was stirred at room temperature under H₂ for 2 h. The reaction mixture was filtered and concentrated under reduced pressure to obtain compound 13-7 (70 mg), which was used in the next step without further purification.

To a mixture of compound 13-7 (70 mg, 0.13 mmol, 1.0 eq.) in THF (5 mL) was added 5.4 N NaOMe (0.1 mL, 0.63 mmol, 5.0 eq.) under N₂. After addition, the mixture was stirred at room temperature for 2 h under N₂. The mixture was concentrated under reduced pressure. The residue was purified by column chromatography on silica gel eluting with eluting with DCM/MeOH (1/0-5:1, v/v) to obtain compound 13-8 (30 mg). ESI-MS m/z: 521.2 [M−H]⁻.

To a mixture of compound 13-8 (30 mg, 0.057 mmol, 1.0 eq.) in DCM (2 mL) was added BBr₃ (0.5 mL) at −78° C. After addition, the mixture was stirred at 35° C. for 4 h under N₂. The mixture was quenched by NH₃·H₂O dropwise at −78° C. to pH-8. The reaction mixture was filtered and concentrated to give crude product, which was purified by prep-HPLC (NH₃·H₂O) to obtain compound 137 (3.41 mg). ESI-MS m/z: 377.2 [M+H]⁺; ¹H NMR (400 MHz, CD₃OD): δ 8.08 (s, 1H), 7.97 (d, J=9.2 Hz, 1H), 7.19 (s, 1H), 6.88 (d, J=9.2 Hz, 1H), 5.06-5.01 (m, 1H), 3.51-3.46 (m, 2H), 1.82-1.72 (m, 1H), 1.66-1.59 (m, 2H), 1.31 (d, J=6.8 Hz, 3H), 1.00 (d, J=6.4 Hz, 6H).

### Example 1.14: Synthesis of 5-(5-fluoro-7-hydroxy-3-(isopentyloxy)-1-methyl-2-oxo-1,2-dihydroquinolin-6-yl)-1,2,5-thiadiazolidin-3-one 1,1-dioxide (136)

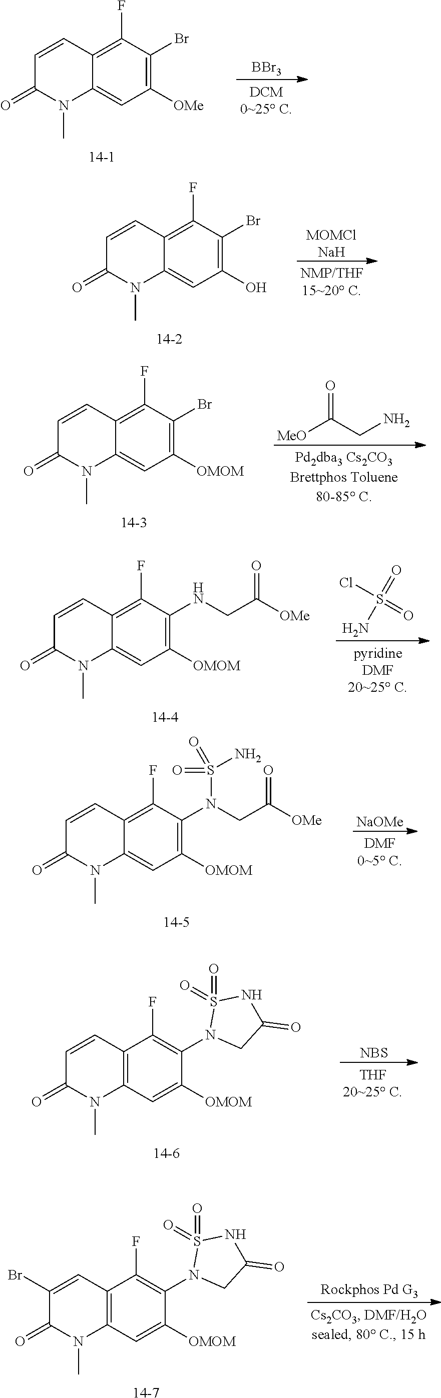

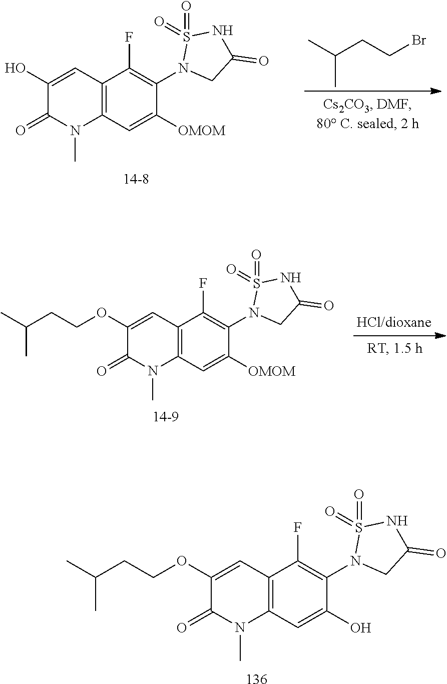

Compound 14-1 was prepared in analogous fashion to the synthesis of compound 5-5 described in Example 5. ¹H NMR (400 MHz, DMSO-d6): δ 7.85 (d, J=9.6 Hz, 1H), 6.85 (s, 1H), 6.51 (d, J=9.6 Hz, 1H), 4.03 (s, 3H), 3.57 (s, 3H).

To a solution of compound 14-1 (20.0 g, 69.9 mmol, 1.00 eq) in DCM (140 mL) was added BBr₃ (105 g, 419 mmol, 40.4 mL, 6.00 eq) at 0˜5° C. under N₂. The resulting mixture was stirred at 20-25° C. for 4 hrs. The reaction mixture was quenched by ice water (500 mL) at 0˜5° C., the mixture solution was filtered and the filtrate cake was concentrated to afford Compound 14-2 (18.0 g) which was used in the next step without further purification. ESI-MS m/z: 271.9 [M+H]⁺.

To a solution of NaH (7.94 g, 199 mmol, 60% purity, 3.00 eq) in THF (54.0 mL) was added a solution of compound 14-2 (18.0 g, 66.2 mmol, 1.00 eq) in NMP (180 mL) at 15-20° C. under N₂. The resulting mixture was stirred at 15-20° C. for 1 hr. MOMCl (7.99 g, 99.2 mmol, 7.54 mL, 1.50 eq) in THF (54.0 mL) was added to the above solution at 15-20° C. The resulting mixture was stirred at 15-20° C. for 3 hrs. The reaction mixture was quenched by ice water (600 mL) at 0-5° C., and extracted with EtOAc (200 mL, 150 mL). The combined organic layers were washed with brine (500 mL), dried over Na₂SO₄, filtered and concentrated under reduced pressure to give a residue. The crude product was triturated with MTBE (36.0 mL) at 20° C. for 30 mins, filtered and the filter cake was washed by MTBE (10.0 mL) to afford compound 14-3 (12.0 g). ESI-MS m/z: 317.9 [M+H]⁺.

To a solution of compound 14-3 (4.00 g, 12.7 mmol, 1.00 eq) in toluene (200 mL) was added methyl glycinate (2.25 g, 25.3 mmol, 2.00 eq) and Cs₂CO₃ (12.4 g, 38.0 mmol, 3.00 eq) at 15-20° C. under N₂. The solution was degassed and purged with N₂ for 3 times. Pd₂(dba)₃ (2.32 g, 2.53 mmol, 0.20 eq) and Brettphos (1.36 g, 2.53 mmol, 0.20 eq) were added to the above solution at 15˜20° C. under N₂. The solution was degassed and purged with N₂ for 3 times. The resulting mixture was stirred at 80˜85° C. for 12 hrs.

Three Reactions were Combined for Workup.

The reaction mixture was quenched by ice water (500 mL) at 0˜5° C., and extracted with EtOAc (300 mL, 200 mL). The combined organic layers were washed with brine (500 mL), dried over Na₂SO₄, filtered and concentrated under reduced pressure to give a residue. The residue was purified by column chromatography (SiO₂, Petroleum ether: Ethyl acetate=100: 1 to 0: 1) to afford compound 14-4 (5.00 g).

To a solution of compound 14-4 (5.00 g, 15.4 mmol, 1.00 eq) in DMF (50.0 mL) was added pyridine (6.10 g, 77.1 mmol, 6.22 mL, 5.00 eq) and sulfamoyl chloride (8.91 g, 77.1 mmol, 5.00 eq) at 0-5° C. The mixture was stirred at 20-25° C. for 2 hrs. The reaction mixture containing compound 14-5 was used in next step without further work up.

To a solution of compound 14-5 (5.00 g, 12.4 mmol, 1.00 eq) in DMF (50.0 mL) was added MeONa (13.4 g, 74.4 mmol, 30% purity, 6.00 eq) at 0˜5° C. The mixture was srirred at 0-5° C. for 2 hrs. The residue was poured to ice water (200 mL). The aqueous phase was extracted with EtOAc (100 mL, 80.0 mL). The combined organic phase was washed with brine (200 mL), dried with Na₂SO₄, filtered and concentrated under reduced pressure to give a residue. The crude product was purified by triturated with EtOAc (10.0 mL) at 20° C. for 30 mins, filtered and the filter cake was washed by EtOAc (4.00 mL) to afford compound 14-6 (2.20 g). ¹H NMR (400 MHz, DMSO-d6): δ 7.95 (s, 1H), 6.96 (s, 1H), 6.55 (d, J=9.6 Hz, 1H), 5.43 (s, 2H), 4.39 (s, 2H), 3.59 (s, 3H), 3.47 (s, 3H).

To a solution of compound 14-6 (2.20 g, 5.92 mmol, 1.00 eq) in THF (22.0 mL) was added NBS (1.58 g, 8.89 mmol, 1.50 eq) at 0-5° C. The mixture was stirred at 0-5° C. for 2 hrs. The reaction mixture was quenched by ice water (50.0 mL) and adjusted pH to 2-3 with 2M HCl at 5-10° C., then extracted with EtOAc (30.0 mL, 20.0 mL). The combined organic layers were washed with brine (50.0 mL), dried over Na₂SO₄, filtered and concentrated under reduced pressure to give a residue. The residue was purified by column chromatography (SiO₂, Petroleum ether: Ethyl acetate=100: 1 to 0: 1) to give a residue. The crude product was purified by triturated with DCM (4.00 mL) at 20° C. for 30 mins, filtered and the filter cake was washed by DCM (2.00 mL) to afford compound 14-7 (1.20 g). ¹H NMR (400 MHz, DMSO-d6): δ 8.44 (s, 1H), 6.98 (s, 1H), 5.46 (s, 2H), 4.48 (s, 2H), 3.68 (s, 3H), 3.45 (s, 3H).

To a mixture of compound 14-7 (50 mg, 0.111 mmol, 1.0 eq.) and Cs₂CO₃ (108 mg, 0.333 mmol, 3.0 eq.) in DMF (2.5 mL) and water (0.05 mL) was added Rockphos Pd G₃ (28 mg, 33.3 μmol, 0.3 eq.) at room temperature. The reaction mixture was stirred at 80° C. under Ar in a sealed tube for 15 h. Two reactions were carried out in parallels. The mixture was concentrated under reduced pressure. The residue was purified by column chromatography on silica gel with MeOH/EtOAc (0-50%) to afford compound 14-8 (71 mg). ESI-MS m/z: 386.1 [M−H]⁻.

To a mixture of compound 14-8 (30 mg, 0.078 mmol, 1.0 eq.) and Cs₂CO₃ (38 mg, 0.117 mmol, 1.5 eq.) in anhydrous DMF (1.5 mL) was added 1-bromo-3-methylbutane (14 mg, 0.093 mmol, 1.2 eq.) at room temperature. The reaction mixture was stirred at 80° C. under Ar in a sealed tube for 2 h. The reaction mixture was concentrated under reduced pressure. The residue was purified by column chromatography on silica gel with MeOH/EtOAc (0-50%) to obtain compound 14-9 (22 mg). ESI-MS m/z: 456.1 [M−H]⁻.

To a solution of HCl/dioxane (4 M, 0.6 mL, 2.40 mmol, 41.7 eq.) was added compound 14-9 (22 mg, 0.048 mmol, 1.0 eq.). After addition, the mixture was stirred at room temperature for 1.5 h. The reaction mixture was diluted with DCM (10 mL) and adjusted to pH=8-9 with NH₃·H₂O at 0° C. The resultant mixture was concentrated in vacuo to give crude product, which was purified by prep-HPLC (NH₄HCO₃) to obtain 136 (5.66 mg). ESI-MS m/z: 412.1 [M−H]⁻; ¹H NMR (400 MHz, CD₃OD): δ 7.18 (s, 1H), 6.80 (s, 1H), 4.34 (s, 2H), 4.08 (t, J=6.6 Hz, 2H), 3.69 (s, 3H), 1.93-1.85 (m, 1H), 1.76 (q, J=6.8 Hz, 2H), 0.99 (d, J=6.4 Hz, 6H).

### Example 1.15: Synthesis of 5-(1,1-dioxido-4-oxo-1,2,5-thiadiazolidin-2-yl)-4-fluoro-6-hydroxy-N-isopentylbenzo[b]thiophene-2-carboxamide (130)

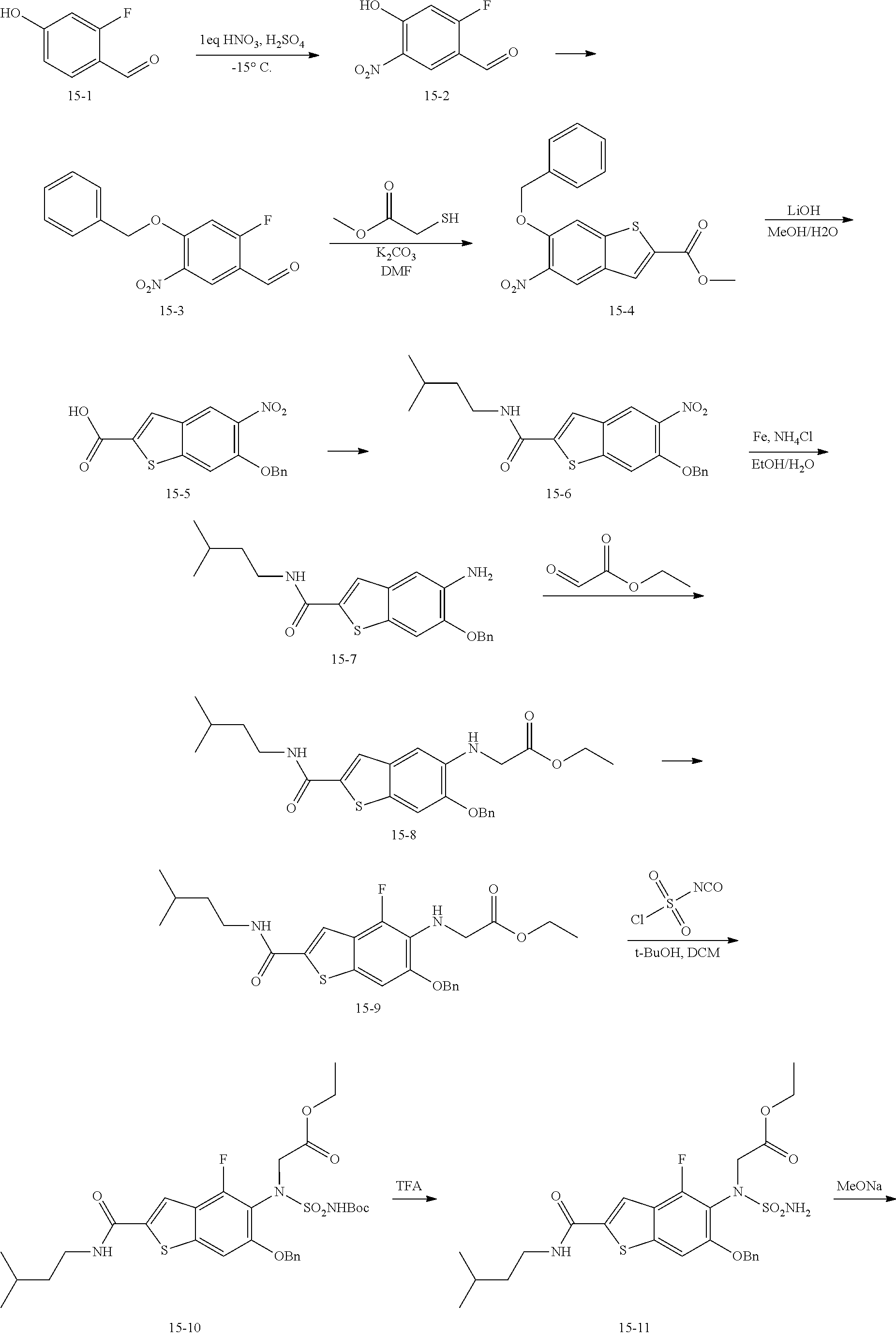

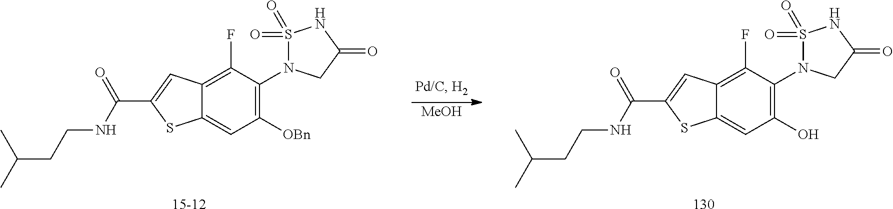 [SMILES: "MeOH" ; CC(C)CCNC(=O)c1cc2c(F)c(N3CC(=O)NS3(=O)=O)c(OCc3ccccc3)cc2s1 ; CC(C)CCNC(=O)c1cc2c(F)c(N3CC(=O)NS3(=O)=O)c(O)cc2s1 ; "15-12" ; "130"]

To a solution of 2-fluoro-4-hydroxybenzaldehyde (15-1) (5.8 g) in conc. H₂SO₄(60 mL) was added conc. HNO₃ (3.6 g, 1.0 eq) slowly at −15° C. The resulting mixture was stirred for 2 h at −15° C. The reaction mixture was poured into ice-water and extracted with EA, washed with brine and dried over Na₂SO₄. The solvent was concentrated to afford compound 15-2 (7.3 g). ESI-MS m/z: 184.02 [M−H]⁻.

To a solution of compound 15-2 (6.5 g, 35.1 mmol) in DMF (100 mL) was added K₂CO₃ (7.3 g, 52.2 mmol), followed by BnBr (7.2 g, 42.9 mmol) at 0° C. The resulting mixture was heated to 45° C. and stirred for 8 h. The reaction mixture was extracted with EA, washed with water and brine, dried over Na₂SO₄, concentrated and purified by flash chromatography on silica gel to afford the compound 15-3 (5.8 g).

To a solution of compound 15-3 (2 g, 7.25 mmol) in DMF (100 mL) was added K₂CO₃ (2.5 g, 18.2 mmol), followed by methyl 2-mercaptoacetate (1.5 g, 14.5 mmol) at 35° C. The resulting mixture was heated to 80° C. and stirred for 16 h. The reaction mixture was extracted with EA, washed with brine, dried over Na₂SO₄, concentrated and purified by flash chromatography on silica gel to afford compound 15-4 (1.02 g).

To a solution of compound 15-4 (500 mg, 1.46 mmol) in MeOH/H₂O (10:1) was added LiOH (184 mg, 4.37 mmol) at 30° C. The resulting mixture was heated to 65° C. and stirred for 2 h. The reaction mixture was acidified by 2N HCl, extracted with EA and dried over Na₂SO₄, and concentrated to afford compound 15-5 (300 mg). ESI-MS m/z: 328.05[M−H]⁻.

To a solution of compound 15-5 (800 mg, 2.42 mmol) in DMF (30 mL) was added HBTU (1.1 g, 2.91 mmol), followed by DIEA (627 mg, 4.85 mmol) and 3-methylbutan-1-amine (422 mg, 4.85 mmol). The resulting mixture was stirred at 35° C. for 8 h. The reaction mixture was extracted with EA, washed with water and brine, dried over Na₂SO₄, concentrated and purified by flash chromatography on silica gel to afford compound 15-6 (1.2 g).

To a solution of compound 15-6 (1.4 g, 3.5 mmol) in EtOH/H₂O (50 mL/12 mL) was added NH₄Cl (1.2 g, 28.1 mmol), followed by Iron powder (786 mg, 14 mmol) slowly, then the resulting mixture was heated to 80° C. and stirred for 3 h. The reaction mixture was concentrated and purified by flash chromatography on silica gel (50% PE/EA) to afford compound 15-7 (1.0 g). ESI-MS m/z: 369.18 [M+H].

To a solution of compound 15-7 (1.0 g, 2.7 mmol) and ethyl 2-oxoacetate (1.66 g, 8.15 mmol) in MeOH (40 mL), was added AcOH (245 mg, 4.08 mmol), followed by NaBH₃CN (685 mg, 11 mmol) slowly, then the resulting mixture was heated to 30° C. and stirred for 8 h. The reaction mixture was concentrated and purified by flash chromatography on silica gel to afford compound 15-8 (1.0 g). ESI-MS m/z: 455.21 [M+H]⁺.

To a solution of compound 15-8 (500 mg, 1.11 mmol) in THF (40 mL), was added NFSI (385 mg, 1.22 mmol) in one portion. The resulting mixture was stirred for 8 h. The reaction mixture was diluted and quenched by Na₂S2O₃ (aq), extracted with EA, washed with brine, dried over Na₂SO₄, concentrated and purified by flash chromatography on silica gel to afford compound 15-9 (260 mg). ESI-MS m/z: 473.19 [M+H]⁺.

To a mixture of sulfurisocyanatidic chloride (450 mg, 3.18 mmol, 10.0 eq.) in DCM (5 mL) was added ^{t}BuOH (236 mg, 3.18 mmol, 10.0 eq.) at 0° C. under N₂. The reaction mixture was stirred at 0° C. under N₂ for 0.5 h. Then a solution of compound 15-9 (150 mg, 0.318 mmol, 1 eq.) and TEA (0.9 mL, 6.36 mmol, 20.0 eq.) in DCM (5 mL) was added to the mixture at 0° C. under N₂. The reaction mixture was stirred at room temperature for 3 h under N₂. The reaction mixture was quenched with H₂O (15 mL) and extracted with DCM (3×15 mL). The combined organic layers were dried over anhydrous Na₂SO₄, filtered and concentrated under reduced pressure to obtain crude compound 15-10 (207 mg), which was used for the next step without further purification. ESI-MS m/z: 552.0 [M−99]⁺.

To a mixture of compound 15-10 (207 mg, 0.318 mmol, 1 eq.) in DCM (6 mL) was added TFA (2 mL). The mixture was stirred at room temperature for 0.5 h. The mixture was adjusted with pH=8-9 with aq. NaHCO₃ solution and extracted with DCM (3×15 mL). The combined organic layers were dried over anhydrous Na₂SO₄, filtered and concentrated under reduced pressure to give compound 15-11 (175 mg), which was used for the next step without further purification. ESI-MS m/z: 552.0 [M+H]⁻.

To a mixture of compound 15-11 (175 mg, 0.318 mmol, 1.0 eq.) and 4A MS (200 mg) in THF (5 mL) and MeOH (1 mL) was added NaOMe soltuon (0.3 mL, 1.59 mmol, 5.0 eq., 5.4 M in MeOH) under N₂. After addition, the mixture was stirred at room temperature for 1 h under N₂. The mixture was concentrated under reduced pressure. The residue was purified by column chromatography on silica gel eluting with eluting with EtOAc/MeOH (1/0-3/1, v/v) to obtain compound 15-12 (70 mg). ESI-MS m/z: 504.0 [M−H]⁻.

To a mixture of compound 15-12 (60 mg, 0.12 mmol, 1.0 eq.) in MeOH (3 mL) was added Pd/C (60 mg, 10% wt, wet). After addition, the mixture was stirred at room temperature for 1 h under H₂ (30 psi). The mixture was filtered and the filter cake was washed with MeOH (10 mL). The filtrate was concentrated under reduced pressure. The residue was purified by column chromatography on silica gel eluting with eluting with EtOAc/MeOH (1/0-10/1, v/v) and prep-HPLC separation to afford compound 130 (3.43 mg). ESI-MS m/z: 414.0 [M−H]⁻; ¹H NMR (400 MHz, CD₃OD): δ 7.91 (s, 1H), 7.18 (s, 1H), 4.34 (s, 2H), 3.46-3.37 (m, 211), 1.78-1.61 (m, 1H), 1.58-1.48 (m, 2H), 0.97 (d, J=6.6 Hz, 611).

### Example 2: A Phosphatase Activity Assay

Assessing Selectivity and Potency of a Small Molecule PTPN2 Inhibitor

The selectivity and potency of a small molecule PTPN2 inhibitor as provided herein (e.g., a derivative of Formula I, II, III, IV; or sub-formulae thereof) against one or more protein tyrosine phosphatase (PTP) enzymes, and specifically against PTPN2, can be assessed in various ways. The one or more PTP enzymes comprise Mycobacterium protein tyrosine phosphatase A (mPTPA), Mycobacterium protein tyrosine phosphatase B (mPTPB), PTPN1 (i.e., PTP1B), PTPN2 (i.e., TC-PTP), SHP-1, SHP-2, FAP-1, Meg2, HePTP, Laforin, VHX, VHR, LMWPTP, Cdcl4A, LAR, CD45, PTPRG, a fragment thereof, a variant thereof, and a combination thereof. Selectivity and potency of a small molecule PTPN2 inhibitor is evaluated using a PTP activity inhibition assay. The assay is performed using a buffer comprising 50 millimolar (mM) Bis-Tris (pH=6.3), 2 mM EDTA, and 5 mM N,N′-dimethyl-N,N′-bis(mercaptoacetyl)hydrazine (DMH). The assay is performed using a phosphated substrate, e.g., 10 mM fluorescein diphosphate (FDP) that is stored at −20° C. Alternatively or in addition to, the assay is performed using 10 mM DiFMUP as a phosphated substrate. Each PTP enzyme is diluted in an enzyme dilution buffer comprising 50 mM Bis-Tris (pH=6.3), 2 mM EDTA, 5 mM DMH, 20% (vol./vol.) glycerol, and 0.01% (vol./vol.) Triton X-100.

The assay is carried out at room temperature in 96 well plates. A reaction mixture of 170 microliter (μL) of the assay buffer comprising 10 μM FDP or DiFMIUP is added to each well. A 10 μL volume of (i) a compound disclosed herein (e.g., PTPN2 inhibitor) dissolved in DMSO at one of 10 concentrations from a serial dilution or (ii) DMSO alone for control is added to each well. The reaction is initiated by adding 20 μL of a PTP enzyme (e.g., PTPN2) that is diluted in the enzyme dilution buffer. The phosphatase activity of the PTP enzyme is assessed by monitoring appearance of a fluorescent product (e.g., fluorescein monophosphate (FMP) from FDP, or 6,8-difluoro-7-hydroxyl-4-coumarin (DiFMU) from DiFMUP) continuously for about 15 to 30 minutes, using the SpectraMax® Gemini™ XPS Microplate Spectrofluorometer (Molecular Devices®) with excitation of 440 nm and emission at 530 nm (cutoff filter at 525 nm) for FMP and excitation at 360 nm and emission at 450 nm (cutoff filter at 435 nm) for DiFMU. The rate (e.g., the initial rate) of FMP or DiFMU formation is plotted against the concentration of the small molecule PTPN2 inhibitor, and the data is fitted (e.g., using a 4-parameter equation) to determine the inflection point of the fit as the IC₅₀ of the small molecule PTPN2 inhibitor for a specific enzyme. Table 6 summarizes the IC50 values of compounds disclosed herein against PTPN2 and PTPN1.

TABLE 6

| | Bio-Biochem- | Bio-Biochem- | | |
|---|---|---|---|---|
| | DiFMUP- | DiFMUP- | Mass | |
| Compound | (PTPN2 IC50) | (PTP1B IC50) | Characterization | |
| number | [nM] | [nM] | [M + H]+ | Compound |
| 101 | + | + | 326.15 | 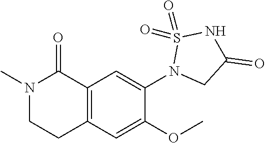 [SMILES: COc1cc2c(cc1N1CC(=O)NS1(=O)=O)C(=O)N(C)CC2] |
| 102 | + | + | 355.15 [M − H]⁻ | 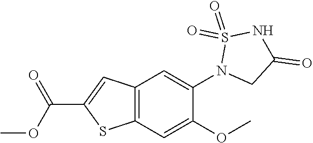 [SMILES: COC(=O)c1cc2cc(N3CC(=O)NS3(=O)=O)c(OC)cc2s1] |
| 103 | ++ | ++ | 342.0 [M − H]⁻ | 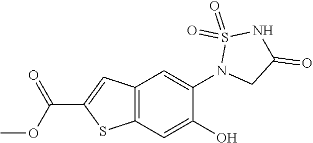 [SMILES: COC(=O)c1cc2cc(N3CC(=O)NS3(=O)=O)c(O)cc2s1] |
| 104 | + | + | 310.25 [M − H]⁻ | 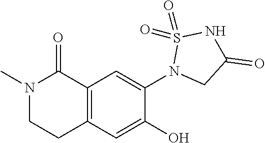 [SMILES: CN1CCc2cc(O)c(N3CC(=O)NS3(=O)=O)cc2C1=O] |
| 105 | ++ | + | 382.4 | 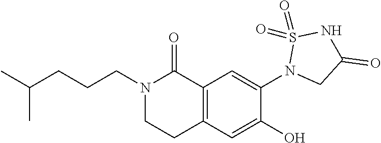 [SMILES: CC(C)CCCN1CCc2cc(O)c(N3CC(=O)NS3(=O)=O)cc2C1=O] |
| 106 | ++ | +++ | 362.1 | 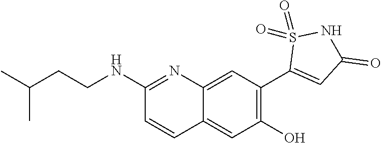 [SMILES: CC(C)CCNc1ccc2cc(O)c(C3=CC(=O)NS3(=O)=O)cc2n1] |
| 107 | + | ++ | 310.9 | 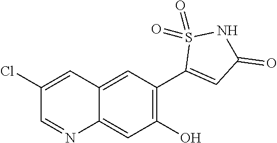 [SMILES: O=C1C=C(c2cc3cc(Cl)cnc3cc2O)S(=O)(=O)N1] |
| 108 | + | + | 380.2 | 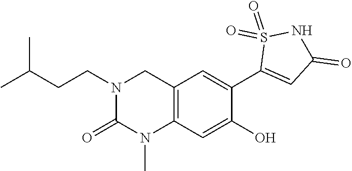 [SMILES: CC(C)CCN1Cc2cc(C3=CC(=O)NS3(=O)=O)c(O)cc2N(C)C1=O] |
| 109 | + | ++ | 364.1 | 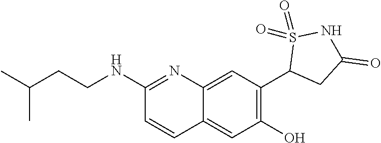 [SMILES: CC(C)CCNc1ccc2cc(O)c(C3CC(=O)NS3(=O)=O)cc2n1] |
| 110 | ++ | ++ | 365.2 | 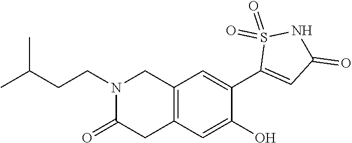 [SMILES: CC(C)CCN1Cc2cc(C3=CC(=O)NS3(=O)=O)c(O)cc2CC1=O] |
| 111 | + | + | 382.2 | 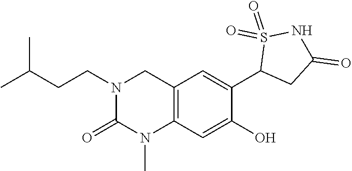 [SMILES: CC(C)CCN1Cc2cc(C3CC(=O)NS3(=O)=O)c(O)cc2N(C)C1=O] |
| 112 | + | + | 367.2 | 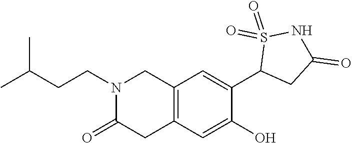 [SMILES: CC(C)CCN1Cc2cc(C3CC(=O)NS3(=O)=O)c(O)cc2CC1=O] |
| 113 | +++ | +++ | 365.0 | 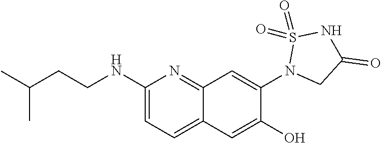 [SMILES: CC(C)CCNc1ccc2cc(O)c(N3CC(=O)NS3(=O)=O)cc2n1] |
| 114 | + | + | 372.0 | 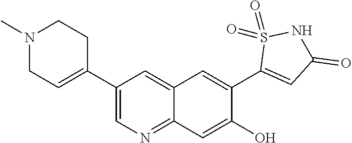 [SMILES: CN1CC=C(c2cnc3cc(O)c(C4=CC(=O)NS4(=O)=O)cc3c2)CC1] |
| 115 | + | ++ | 354.0 | 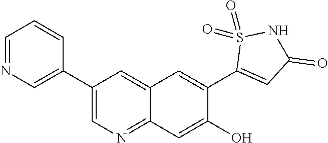 [SMILES: O=C1C=C(c2cc3cc(-c4cccnc4)cnc3cc2O)S(=O)(=O)N1] |
| 116 | ++ | +++ | 383.2 | 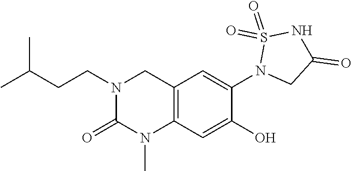 [SMILES: CC(C)CCN1Cc2cc(N3CC(=O)NS3(=O)=O)c(O)cc2N(C)C1=O] |
| 117 | +++ | +++ | 381.0 [M − H]⁻ | 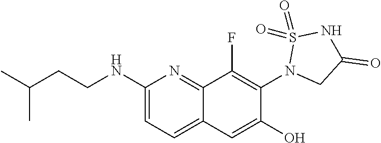 [SMILES: CC(C)CCNc1ccc2cc(O)c(N3CC(=O)NS3(=O)=O)c(F)c2n1] |
| 118 | +++ | +++ | 399.0 | 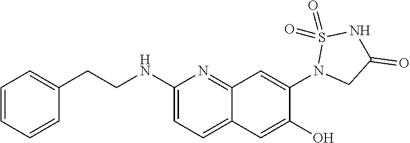 [SMILES: O=C1CN(c2cc3nc(NCCc4ccccc4)ccc3cc2O)S(=O)(=O)N1] |
| 119 | +++ | +++ | 368.0 | 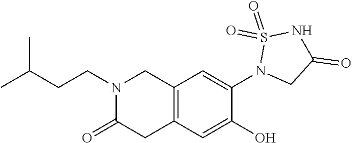 [SMILES: CC(C)CCN1Cc2cc(N3CC(=O)NS3(=O)=O)c(O)cc2CC1=O] |
| 120 | +++ | +++ | 393.0 [M − H]⁻ | 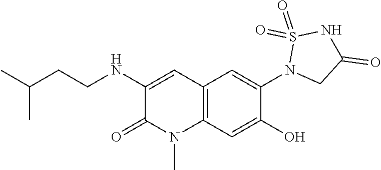 [SMILES: CC(C)CCNc1cc2cc(N3CC(=O)NS3(=O)=O)c(O)cc2n(C)c1=O] |
| 121 | ++ | ++ | 379.1 | 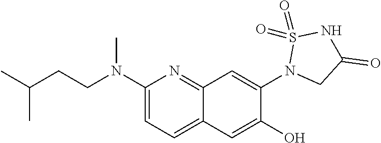 [SMILES: CC(C)CCN(C)c1ccc2cc(O)c(N3CC(=O)NS3(=O)=O)cc2n1] |
| 122 | + | + | 392.0 | 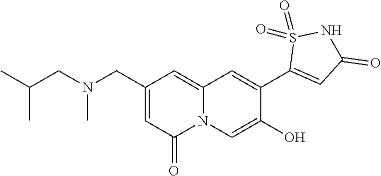 [SMILES: CC(C)CN(C)Cc1cc(=O)n2cc(O)c(C3=CC(=O)NS3(=O)=O)cc2c1] |
| 123 | +++ | +++ | 427.0 [M − H]⁻ | 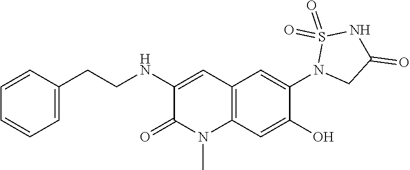 [SMILES: Cn1c(=O)c(NCCc2ccccc2)cc2cc(N3CC(=O)NS3(=O)=O)c(O)cc21] |
| 124 | +++ | +++ | 307.9 [M − H]⁻ | 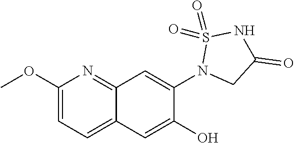 [SMILES: COc1ccc2cc(O)c(N3CC(=O)NS3(=O)=O)cc2n1] |
| 125 | +++ | +++ | 417.1 | 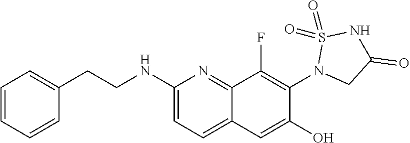 [SMILES: O=C1CN(c2c(O)cc3ccc(NCCc4ccccc4)nc3c2F)S(=O)(=O)N1] |
| 126 | +++ | +++ | 400.3 | 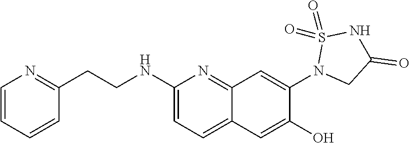 [SMILES: O=C1CN(c2cc3nc(NCCc4ccccn4)ccc3cc2O)S(=O)(=O)N1] |
| 127 | +++ | +++ | 436.1 [M − H]⁻ | 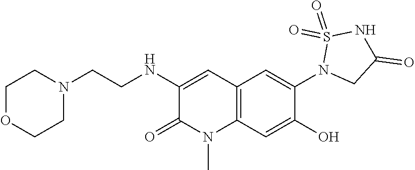 [SMILES: Cn1c(=O)c(NCCN2CCOCC2)cc2cc(N3CC(=O)NS3(=O)=O)c(O)cc21] |
| 128 | ND | ND | 408.1 | 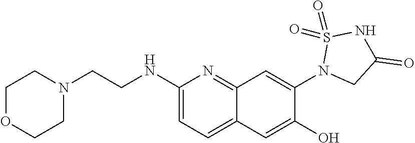 [SMILES: O=C1CN(c2cc3nc(NCCN4CCOCC4)ccc3cc2O)S(=O)(=O)N1] |
| 129 | ND | ND | 419.2 [M − H]⁻ | 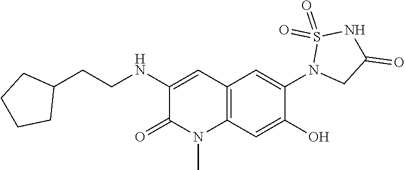 [SMILES: Cn1c(=O)c(NCCC2CCCC2)cc2cc(N3CC(=O)NS3(=O)=O)c(O)cc21] |
| | Bio-Biochem- | Bio-Biochem- | | |
| | DiFMUP- | DiFMUP- | Mass | |
| Compound | (PTPN2 IC50) | (PTP1B IC50) | Characterization | |
| number | [nM] | [nM] | [M − H]⁻ | Compound |
| 130 | +++ | +++ | 414 | 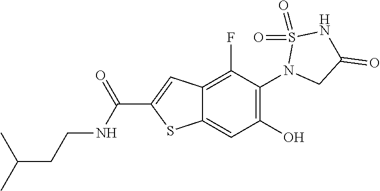 [SMILES: CC(C)CCNC(=O)c1cc2c(F)c(N3CC(=O)NS3(=O)=O)c(O)cc2s1] |
| 131 | + | + | 407.1 | 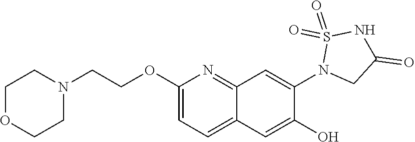 [SMILES: O=C1CN(c2cc3nc(OCCN4CCOCC4)ccc3cc2O)S(=O)(=O)N1] |
| 132 | +++ | +++ | 396.3 | 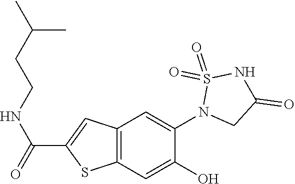 [SMILES: CC(C)CCNC(=O)c1cc2cc(N3CC(=O)NS3(=O)=O)c(O)cc2s1] |
| 133 | +++ | +++ | 409.1 | 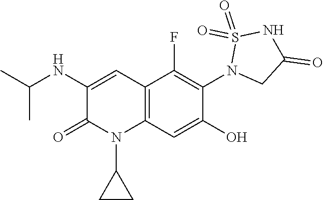 [SMILES: CC(C)Nc1cc2c(F)c(N3CC(=O)NS3(=O)=O)c(O)cc2n(C2CC2)c1=O] |
| 134 | ++ | +++ | 377 | 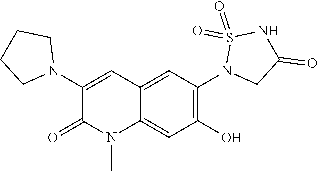 [SMILES: Cn1c(=O)c(N2CCCC2)cc2cc(N3CC(=O)NS3(=O)=O)c(O)cc21] |
| 135 | +++ | +++ | 382.2 | 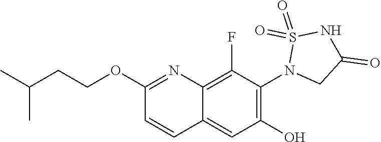 [SMILES: CC(C)CCOc1ccc2cc(O)c(N3CC(=O)NS3(=O)=O)c(F)c2n1] |
| 136 | +++ | +++ | 412.1 | 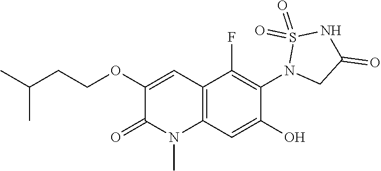 [SMILES: CC(C)CCOc1cc2c(F)c(N3CC(=O)NS3(=O)=O)c(O)cc2n(C)c1=O] |
| 137 | +++ | +++ | 377.2 | 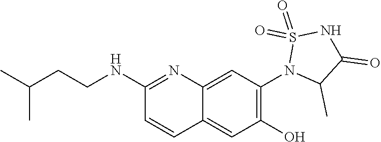 [SMILES: CC(C)CCNc1ccc2cc(O)c(N3C(C)C(=O)NS3(=O)=O)cc2n1] |
| 138 | +++ | +++ | 379.1 | 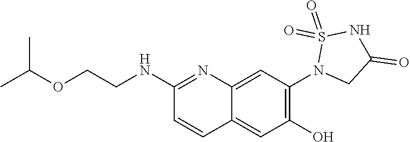 [SMILES: CC(C)OCCNc1ccc2cc(O)c(N3CC(=O)NS3(=O)=O)cc2n1] |
| 139 | +++ | +++ | 396.3 | 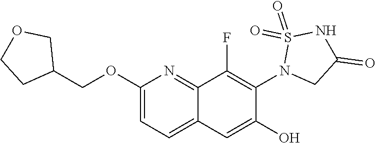 [SMILES: O=C1CN(c2c(O)cc3ccc(OCC4CCOC4)nc3c2F)S(=O)(=O)N1] |
| 140 | ++ | +++ | 435.1 | 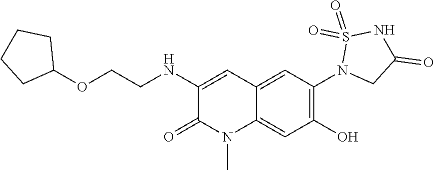 [SMILES: Cn1c(=O)c(NCCOC2CCCC2)cc2cc(N3CC(=O)NS3(=O)=O)c(O)cc21] |
| 141 | +++ | +++ | 433.1 | 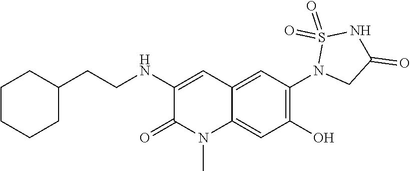 [SMILES: Cn1c(=O)c(NCCC2CCCCC2)cc2cc(N3CC(=O)NS3(=O)=O)c(O)cc21] |
| 142 | +++ | +++ | 363.1 | 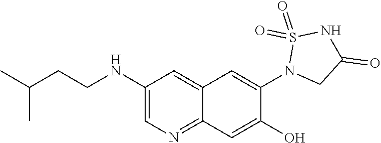 [SMILES: CC(C)CCNc1cnc2cc(O)c(N3CC(=O)NS3(=O)=O)cc2c1] |
| 143 | +++ | +++ | 458 | 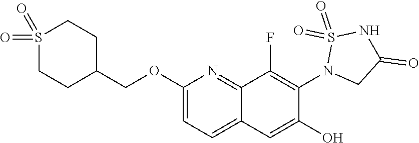 [SMILES: O=C1CN(c2c(O)cc3ccc(OCC4CCS(=O)(=O)CC4)nc3c2F)S(=O)(=O)N1] |
| 144 | +++ | +++ | 410.3 | 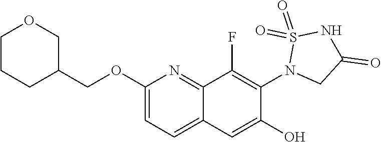 [SMILES: O=C1CN(c2c(O)cc3ccc(OCC4CCCOC4)nc3c2F)S(=O)(=O)N1] |
| 145 | +++ | +++ | 419.1 | 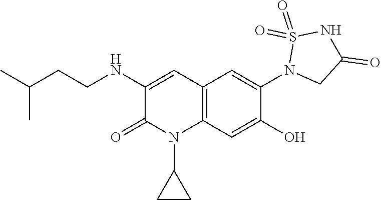 [SMILES: CC(C)CCNc1cc2cc(N3CC(=O)NS3(=O)=O)c(O)cc2n(C2CC2)c1=O] |
| 146 | +++ | +++ | 356.1 | 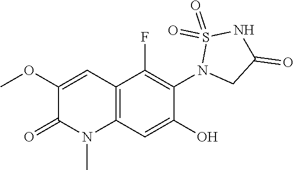 [SMILES: COc1cc2c(F)c(N3CC(=O)NS3(=O)=O)c(O)cc2n(C)c1=O] |
| 147 | ++ | +++ | 348.1 | 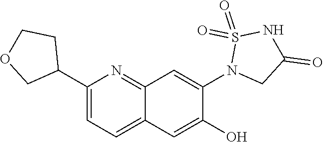 [SMILES: O=C1CN(c2cc3nc(C4CCOC4)ccc3cc2O)S(=O)(=O)N1] |
| 148 | +++ | +++ | 390.1 | 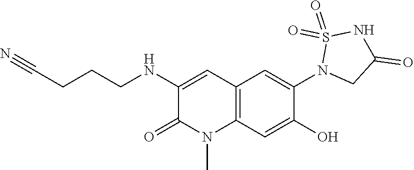 [SMILES: Cn1c(=O)c(NCCCC#N)cc2cc(N3CC(=O)NS3(=O)=O)c(O)cc21] |
| 149 | +++ | +++ | 412.3 | 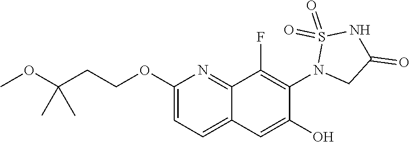 [SMILES: COC(C)(C)CCOc1ccc2cc(O)c(N3CC(=O)NS3(=O)=O)c(F)c2n1] |
| 150 | +++ | +++ | 396.2 | 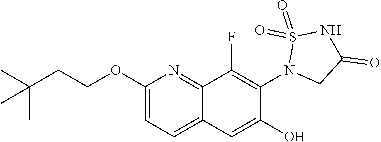 [SMILES: CC(C)(C)CCOc1ccc2cc(O)c(N3CC(=O)NS3(=O)=O)c(F)c2n1] |
| 151 | +++ | +++ | 416.2 | 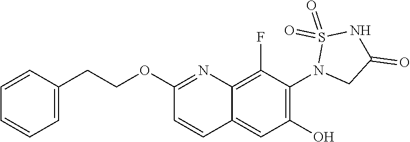 [SMILES: O=C1CN(c2c(O)cc3ccc(OCCc4ccccc4)nc3c2F)S(=O)(=O)N1] |
| 152 | +++ | +++ | 392.2 | 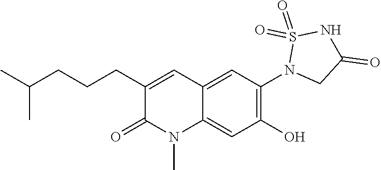 [SMILES: CC(C)CCCc1cc2cc(N3CC(=O)NS3(=O)=O)c(O)cc2n(C)c1=O] |
| 153 | +++ | +++ | 325.2 | 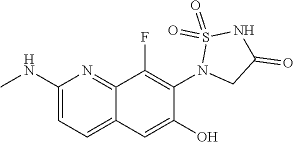 [SMILES: CNc1ccc2cc(O)c(N3CC(=O)NS3(=O)=O)c(F)c2n1] |
| 154 | +++ | +++ | 328.0 [M + H]⁺ | 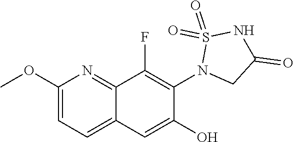 [SMILES: COc1ccc2cc(O)c(N3CC(=O)NS3(=O)=O)c(F)c2n1] |
| 155 | +++ | +++ | 379.3 | 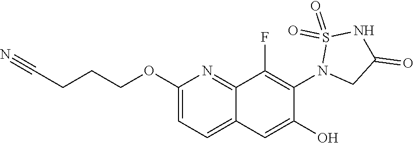 [SMILES: N#CCCCOc1ccc2cc(O)c(N3CC(=O)NS3(=O)=O)c(F)c2n1] |
| 156 | +++ | +++ | 384.1 | 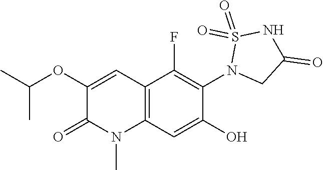 [SMILES: CC(C)Oc1cc2c(F)c(N3CC(=O)NS3(=O)=O)c(O)cc2n(C)c1=O] |
| 157 | ++ | ++ | 382 | 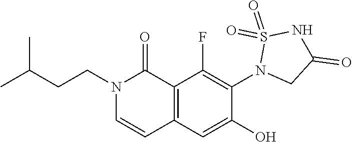 [SMILES: CC(C)CCn1ccc2cc(O)c(N3CC(=O)NS3(=O)=O)c(F)c2c1=O] |
| 158 | +++ | +++ | 408.3 | 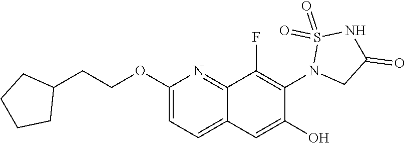 [SMILES: O=C1CN(c2c(O)cc3ccc(OCCC4CCCC4)nc3c2F)S(=O)(=O)N1] |
| 159 | +++ | +++ | 423 | 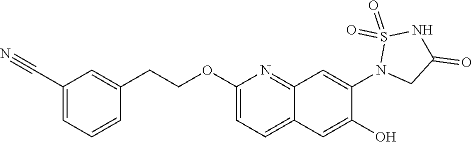 [SMILES: N#Cc1cccc(CCOc2ccc3cc(O)c(N4CC(=O)NS4(=O)=O)cc3n2)c1] |
| 160 | +++ | +++ | 394.2 | 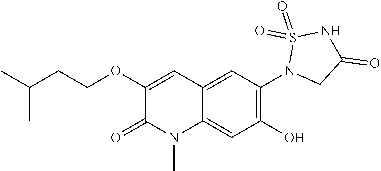 [SMILES: CC(C)CCOc1cc2cc(N3CC(=O)NS3(=O)=O)c(O)cc2n(C)c1=O] |
| 161 | + | + | 333.1 | 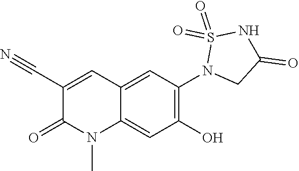 [SMILES: Cn1c(=O)c(C#N)cc2cc(N3CC(=O)NS3(=O)=O)c(O)cc21] |
| 162 | +++ | +++ | 427.1 | 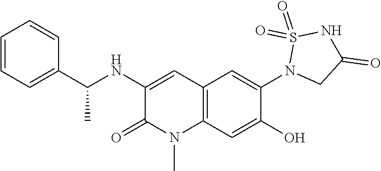 [SMILES: C[C@@H](Nc1cc2cc(N3CC(=O)NS3(=O)=O)c(O)cc2n(C)c1=O)c1ccccc1] |
| 163 | + | + | 377.1 | 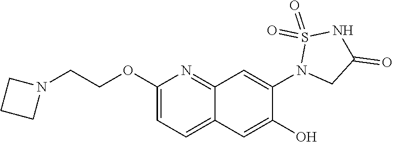 [SMILES: O=C1CN(c2cc3nc(OCCN4CCC4)ccc3cc2O)S(=O)(=O)N1] |
| 164 | +++ | +++ | 376.1 | 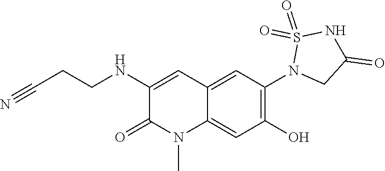 [SMILES: Cn1c(=O)c(NCCC#N)cc2cc(N3CC(=O)NS3(=O)=O)c(O)cc21] |
| 165 | +++ | +++ | 383.1 | 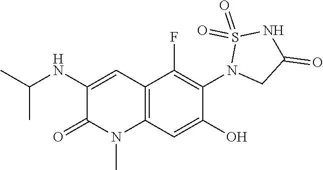 [SMILES: CC(C)Nc1cc2c(F)c(N3CC(=O)NS3(=O)=O)c(O)cc2n(C)c1=O] |
| 166 | ++ | ++ | 436.2 [M + H]⁺ | 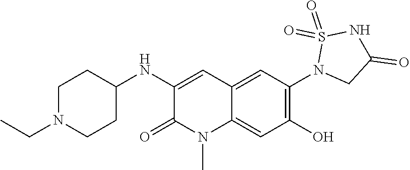 [SMILES: CCN1CCC(Nc2cc3cc(N4CC(=O)NS4(=O)=O)c(O)cc3n(C)c2=O)CC1] |
| 167 | +++ | +++ | 398.1 | 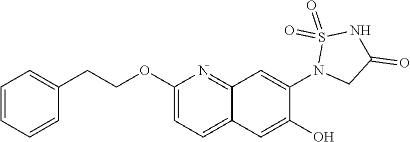 [SMILES: O=C1CN(c2cc3nc(OCCc4ccccc4)ccc3cc2O)S(=O)(=O)N1] |
| 168 | ++ | +++ | 391.1 | 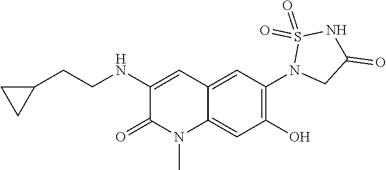 [SMILES: Cn1c(=O)c(NCCC2CC2)cc2cc(N3CC(=O)NS3(=O)=O)c(O)cc21] |
| 169 | ++ | ++ | 391.2 | 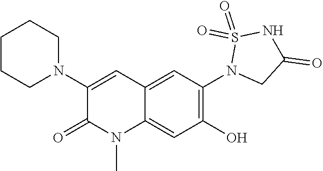 [SMILES: Cn1c(=O)c(N2CCCCC2)cc2cc(N3CC(=O)NS3(=O)=O)c(O)cc21] |
| 170 | +++ | +++ | 430.2 | 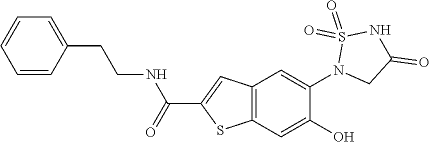 [SMILES: O=C1CN(c2cc3cc(C(=O)NCCc4ccccc4)sc3cc2O)S(=O)(=O)N1] |
| 171 | +++ | +++ | 450.2 [M + H]⁺ | 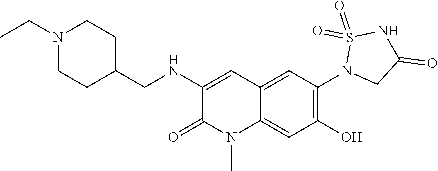 [SMILES: CCN1CCC(CNc2cc3cc(N4CC(=O)NS4(=O)=O)c(O)cc3n(C)c2=O)CC1] |
| 172 | ++ | ++ | 406.1 | 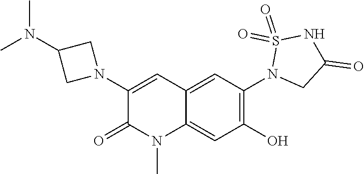 [SMILES: CN(C)C1CN(c2cc3cc(N4CC(=O)NS4(=O)=O)c(O)cc3n(C)c2=O)C1] |
| 173 | +++ | +++ | 401 | 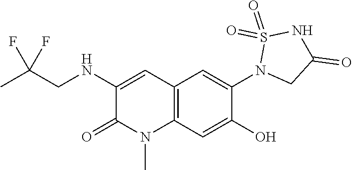 [SMILES: Cn1c(=O)c(NCC(C)(F)F)cc2cc(N3CC(=O)NS3(=O)=O)c(O)cc21] |
| 174 | ++ | +++ | 406.2 | 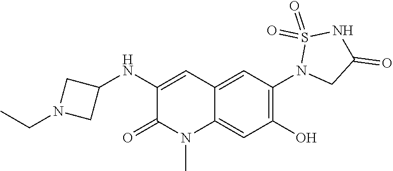 [SMILES: CCN1CC(Nc2cc3cc(N4CC(=O)NS4(=O)=O)c(O)cc3n(C)c2=O)C1] |
| 175 | +++ | +++ | 361 | 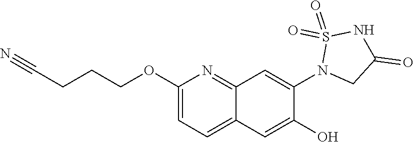 [SMILES: N#CCCCOc1ccc2cc(O)c(N3CC(=O)NS3(=O)=O)cc2n1] |
| 176 | +++ | +++ | 423 | 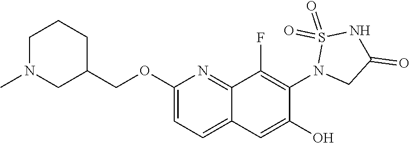 [SMILES: CN1CCCC(COc2ccc3cc(O)c(N4CC(=O)NS4(=O)=O)c(F)c3n2)C1] |
| 177 | +++ | +++ | 413.1 | 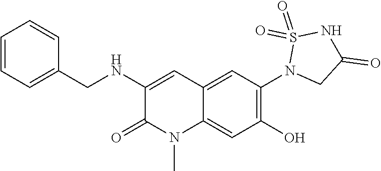 [SMILES: Cn1c(=O)c(NCc2ccccc2)cc2cc(N3CC(=O)NS3(=O)=O)c(O)cc21] |
| 178 | +++ | +++ | 383 | 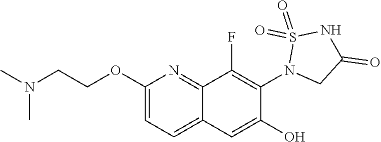 [SMILES: CN(C)CCOc1ccc2cc(O)c(N3CC(=O)NS3(=O)=O)c(F)c2n1] |
| 179 | +++ | +++ | 437.3 | 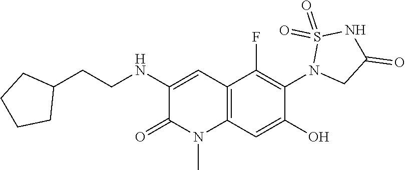 [SMILES: Cn1c(=O)c(NCCC2CCCC2)cc2c(F)c(N3CC(=O)NS3(=O)=O)c(O)cc21] |
| 180 | +++ | +++ | 431.2 | 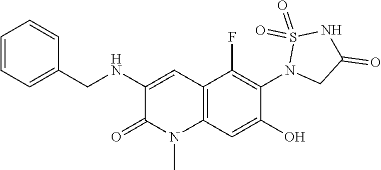 [SMILES: Cn1c(=O)c(NCc2ccccc2)cc2c(F)c(N3CC(=O)NS3(=O)=O)c(O)cc21] |
| 181 | +++ | +++ | 388.1 | 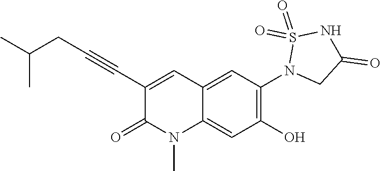 [SMILES: CC(C)CC#Cc1cc2cc(N3CC(=O)NS3(=O)=O)c(O)cc2n(C)c1=O] |
| 182 | +++ | +++ | 364.3 | 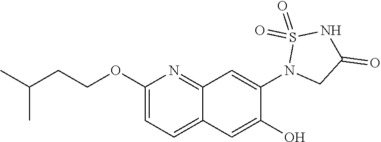 [SMILES: CC(C)CCOc1ccc2cc(O)c(N3CC(=O)NS3(=O)=O)cc2n1] |
| 183 | +++ | +++ | 378.1 | 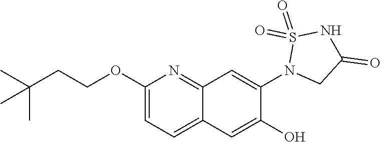 [SMILES: CC(C)(C)CCOc1ccc2cc(O)c(N3CC(=O)NS3(=O)=O)cc2n1] |
| 184 | ++ | +++ | 342.2 | 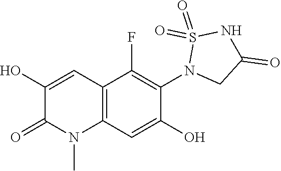 [SMILES: Cn1c(=O)c(O)cc2c(F)c(N3CC(=O)NS3(=O)=O)c(O)cc21] |
| 185 | +++ | +++ | 435.1 | 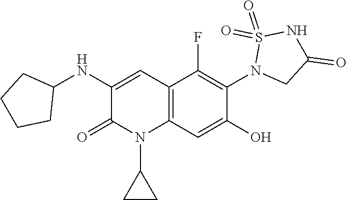 [SMILES: O=C1CN(c2c(O)cc3c(cc(NC4CCCC4)c(=O)n3C3CC3)c2F)S(=O)(=O)N1] |
| 186 | +++ | +++ | 411.1 | 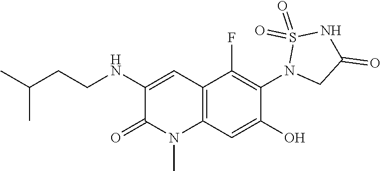 [SMILES: CC(C)CCNc1cc2c(F)c(N3CC(=O)NS3(=O)=O)c(O)cc2n(C)c1=O] |
| 187 | + | + | 349.1 [M + H]+ | 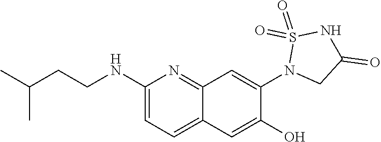 [SMILES: CC(C)CCNc1ccc2cc(O)c(N3CC(=O)NS3(=O)=O)cc2n1] |
| 188 | +++ | +++ | 398.1 | 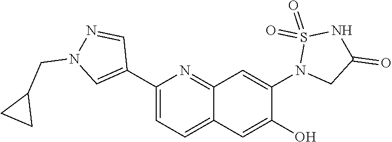 [SMILES: O=C1CN(c2cc3nc(-c4cnn(CC5CC5)c4)ccc3cc2O)S(=O)(=O)N1] |
| 189 | +++ | +++ | 416.2 | 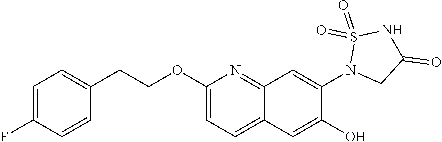 [SMILES: O=C1CN(c2cc3nc(OCCc4ccc(F)cc4)ccc3cc2O)S(=O)(=O)N1] |
| 190 | +++ | +++ | 410.2 | 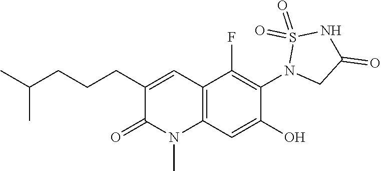 [SMILES: CC(C)CCCc1cc2c(F)c(N3CC(=O)NS3(=O)=O)c(O)cc2n(C)c1=O] |
| 191 | +++ | +++ | 470.1 | 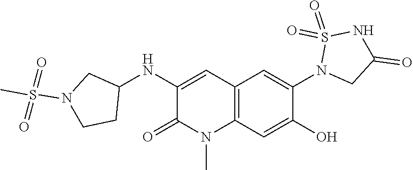 [SMILES: Cn1c(=O)c(NC2CCN(S(C)(=O)=O)C2)cc2cc(N3CC(=O)NS3(=O)=O)c(O)cc21] |
| 192 | +++ | +++ | 354.1 | 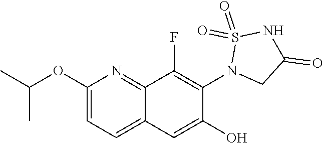 [SMILES: CC(C)Oc1ccc2cc(O)c(N3CC(=O)NS3(=O)=O)c(F)c2n1] |
| 193 | +++ | +++ | 404.2 | 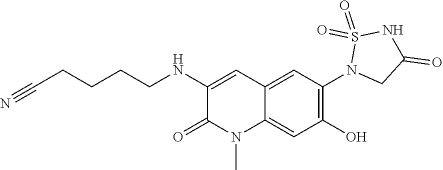 [SMILES: Cn1c(=O)c(NCCCCC#N)cc2cc(N3CC(=O)NS3(=O)=O)c(O)cc21] |
| 194 | ++ | +++ | 427.1 | 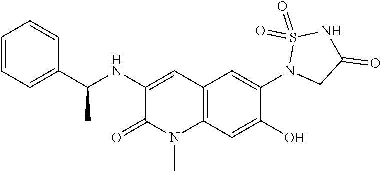 [SMILES: C[C@H](Nc1cc2cc(N3CC(=O)NS3(=O)=O)c(O)cc2n(C)c1=O)c1ccccc1] |
| 195 | +++ | +++ | 409.2 | 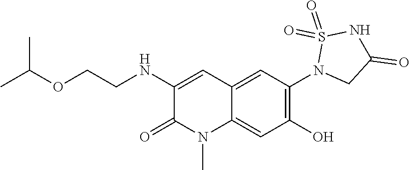 [SMILES: CC(C)OCCNc1cc2cc(N3CC(=O)NS3(=O)=O)c(O)cc2n(C)c1=O] |
| 196 | + | + | 365.2 | 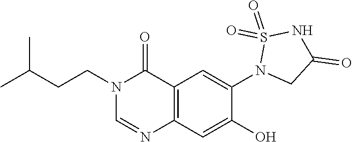 [SMILES: CC(C)CCn1cnc2cc(O)c(N3CC(=O)NS3(=O)=O)cc2c1=O] |
| 197 | +++ | +++ | 362 | 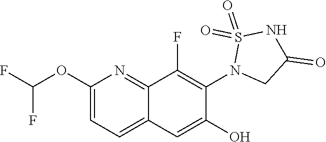 [SMILES: O=C1CN(c2c(O)cc3ccc(OC(F)F)nc3c2F)S(=O)(=O)N1] |
| 198 | +++ | +++ | 448 | 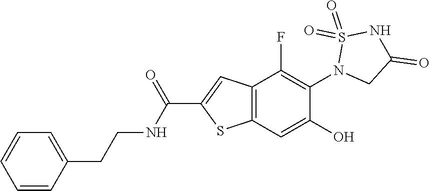 [SMILES: O=C1CN(c2c(O)cc3sc(C(=O)NCCc4ccccc4)cc3c2F)S(=O)(=O)N1] |
| 199 | + | + | 364.2 | 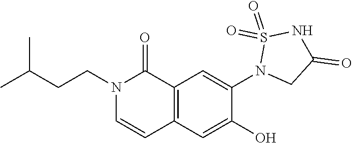 [SMILES: CC(C)CCn1ccc2cc(O)c(N3CC(=O)NS3(=O)=O)cc2c1=O] |
| 200 | +++ | +++ | 410.3 | 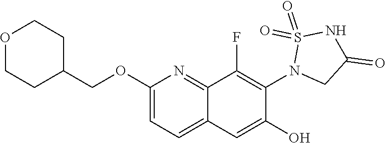 [SMILES: O=C1CN(c2c(O)cc3ccc(OCC4CCOCC4)nc3c2F)S(=O)(=O)N1] |
| 201 | +++ | +++ | 469.2 | 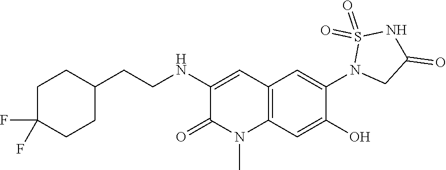 [SMILES: Cn1c(=O)c(NCCC2CCC(F)(F)CC2)cc2cc(N3CC(=O)NS3(=O)=O)c(O)cc21] |
| 202 | ++ | +++ | 402.2 | 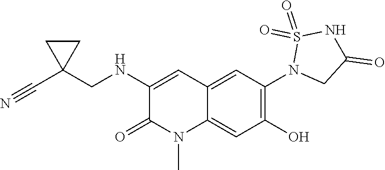 [SMILES: Cn1c(=O)c(NCC2(C#N)CC2)cc2cc(N3CC(=O)NS3(=O)=O)c(O)cc21] |
| 203 | ++ | ++ | 407.1 | 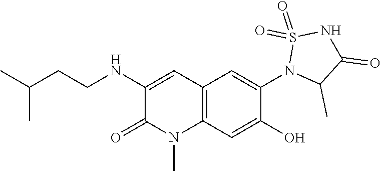 [SMILES: CC(C)CCNc1cc2cc(N3C(C)C(=O)NS3(=O)=O)c(O)cc2n(C)c1=O] |
| 204 | +++ | +++ | 434.1 | 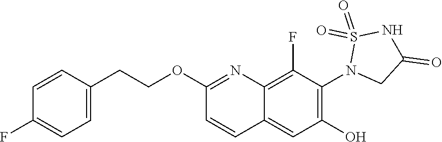 [SMILES: O=C1CN(c2c(O)cc3ccc(OCCc4ccc(F)cc4)nc3c2F)S(=O)(=O)N1] |
| 205 | +++ | +++ | 366.1 | 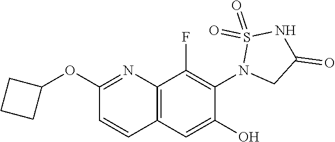 [SMILES: O=C1CN(c2c(O)cc3ccc(OC4CCC4)nc3c2F)S(=O)(=O)N1] |

“+++” ≤ 1 μM; 1 μM < “+” < 10 μM; “+” > 10 μM;
“ND” not determined

### Example 3: Measurement of Increased MHC Expression Associated with Enhanced IFNγ Signaling by PTPN2 Inhibition

B16F10 mouse melanoma cells (ATCC Cat #CRL-6475, Manassas, VA) are seeded at approximately a density of 500 cells per well in a 384-well clear bottom plate (Corning Cat #3765, Corning, 5 NY) in 25 μL total volume of DMEM+10% FBS (Sigma Cat #D6429 and Sigma Cat #F4135, St. Louis, MO). Cells are allowed to adhere overnight at 37° C.+5% CO₂. On the following day, 12.5 μL of mouse IFNγ (RD systems Cat #485-MI/CF, Minneapolis, MN) is added to half of the plate (columns 13-24) at a concentration of 2 ng/mL for a final assay concentration of 0.5 ng/mL of IFNγ. Media only (12.5 μL of DMEM+10% FBS) is added to the remainder of the 10 plate (columns 1-12). Next, compounds resuspended in DMSO (Sigma Cat #D2650) at 100 mM are diluted in semi-log dilutions in DMSO ranging from 100 mM to 0.001 mM and DMSO only controls are included. The compound/DMSO dilutions are further diluted 1:250 in DMEM+10% FBS, and 12.5 μL of these dilutions are added in triplicates to cells of both treatment arms (with and without IFNγ). Compounds are only dosed in the inner 240 wells, avoiding the outer 2-well perimeter of the plate to minimize edge effects. Finally, the plate is loaded into an IncuCyte® S3 Live Cell Analysis System (Essen Bioscience-Sartorius, Ann Arbor, MI) maintained in a 37° C.+5% CO₂ incubator, allowed to equilibrate for 2 hours, and treated for 48 hours. MHC levels are measured by flow cytometry using a FITC anti-mouse H-2kb antibody (clone AF6-88.5), and levels are normalized based on DMSO/with IFNγ control. Table 7 below summarizes the percentage in MHC expression in cells treated with or without compounds disclosed herein at a specified concentration. The results demonstrate that compounds disclosed herein (e.g., PTPN2 inhibitors) are capable of potentiating MHC expression associated with IFN γ signaling.

TABLE 7

| | % MHC in cells | | % MHC in cells |
|---|---|---|---|
| | treated with | | treated with |
| | Compound at 3 uM | | Compound at 3 uM |
| Compound | relative to negative | Compound | relative to negative |
| No. | control (DMSO) | No. | control (DMSO) |
| 117 | +++ | 149 | +++ |
| 151 | ++ | 176 | +++ |
| 135 | +++ | 130 | +++ |
| 183 | < | 143 | +++ |
| 204 | ++ | 178 | +++ |
| 144 | +++ | 186 | +++ |
| 139 | +++ | 190 | ++ |
| 200 | +++ | 154 | +++ |
| 158 | +++ | 153 | +++ |
| 155 | +++ | 197 | +++ |

++ indicates an increase over 1% but less than 10%;
+++ indicates an increase over 10%;
< indicates less than 1%

### Example 4: Enhanced pSTAT1 Signaling by PTPN2 Inhibitors

IFNα signaling in mouse C57BL/6 T cells promotes activation of multiple signaling pathways associated with antitumor responses. A key target of this signaling is phosphorylation of the transcription factor STAT1. The results summarized in Table 8 demonstrates that compounds disclosed herein (e.g., PTPN2 inhibitors) increase pSTAT1 signaling relative to DMSO control. T cells are stimulated for 5 hours by IFNα in the presence or absence of exemplified compounds disclosed herein (e.g., PTPN2 inhibitors) and stained intracellularly with antibodies for phosphorylated STAT1 and then analyzed by flow cytometry. Enhancement of phosphorylation of STAT1 is read out as a percentage over the DMSO control. The results in Table 8 below demonstrate that the exemplified compounds (e.g., PTPN2 inhibitors) are capable of enhancing pSTAT1 signaling as evidenced by an increase in phosphorylated STAT1 when treated with a compound (10 uM) disclosed herein. Many of the exemplified compounds shown below exhibit over 100%, 150%, or 200% pSTAT1 level in cells treated with such exemplary compounds relative to the pSTAT1 level detected in cells treated with negative control (DMSO).

TABLE 8

| | % pSTAT1 in cells treated with | | % pSTATI in cells treated with subject |
|---|---|---|---|
| Compound | subject compounds relative to | Compound | compounds relative to negative control |
| No. | negative control (DMSO) | No. | (DMSO) |
| 117 | 210 | 165 | 139 |
| 135 | 255 | 136 | 131 |
| 176 | 229 | 192 | 183 |
| 143 | 161 | 133 | 170 |
| 178 | 172 | 185 | 181 |
| 154 | 303 | 197 | 196 |
| 153 | 249 | | |

IL2 signaling in T cells promotes T cell homeostasis and proliferation. STAT5 is a signaling molecule in the L2 pathway and a direct target of PTPN2 which serve as negative regulators of L2 signaling. The same procedure outline above in Example 4 can be applied to test for the ability of a compound disclosed herein (e.g., a PTPN2 inhibitor) to potentiate IL2 signaling in T cells.

### Example 5: T Cell Function Assays

T-Cell Activation as Evidenced by Augmented Expression of T-Cell Activation Markers

The increase of T cell activation and most importantly T cell function is a strategy for novel immune oncology approaches to promote tumor immunity. In vitro assays using wildtype C57B3L/6 mouse primary T cells are commonly used to assess the impact of compound on T cell activation and function. To assess the ability of compounds disclosed here (e.g., PTPN2 inhibitors) to stimulate T cell activation, primary T cells stimulated with antibodies to CD3 and CD28 are treated with or without exemplary compounds (at 10 uM) disclosed herein are stained with antibodies directed to T-cell activation markers, such as CD25. Additional markers including without limitation CD69, SLAMF6, TIM3, and PD1 can also be employed as a proxy for T cell activation. The results summarized in Table 9 below demonstrate that exemplary compounds of the disclosure are capable of augmenting CD25 expression on primary T cells. Many of the exemplified compounds shown below exhibit more than 100%, 150%, 200%, 300%, or 400%, CD25 expression in cells treated with such exemplary compounds relative to that of cells treated with negative control (DMSO).

TABLE 9

| | % CD25 expression in cells treated with |
|---|---|
| Compound | subject compounds relative to negative control |
| No. | (DMSO) |
| 117 | 363 |
| 135 | 431 |
| 176 | 206 |
| 143 | 323 |
| 192 | 375 |
| 205 | 423 |
| 197 | 436 |

T-Cell Activation as Evidenced by Enhanced Intracellular Production of IFNγ and TNFα

1*10⁶ OT-1 cells are seeded in a 24well in a volume of 1 mL of RPMI/10% FCS, and activated with 10 nM OVA protein for 48 h. Cells are then split 1:4 into 96well plates after two washes in warm media. To induce exhaustion, cells are then expanded for 3 days in the presence of vehicle control or a compound described herein (e.g., PTPN2 inhibitor) various concentrations, as well as 5 ng/ml IL-7 and 5 ng/ml IL-15, with the addition of fresh OVA on each day. After this point, cells are treated with Brefeldin A and restimulated with 1 μM of OVA for 4-6h, and cells are stained to assess production of IFNγ and TNFα intracellularly by flow cytometry. The results are shown as increase in percentage production of IFNγ and TNFα in cells treated with an exemplified compound at 10 uM, relative to a negative control (DMSO) of cells without being treated with the compound but merely DMSO (a solvent in which exemplified compounds are dissolved). See Table 10 below summarizes percentage increase in IFNγ and TNFα intracellular production in cells treated with an exemplified compound (at 10 uM) or without it, relative to a negative control (DMSO). Many of the exemplified compounds shown below exhibit more than 100%, 150%, 200%, 300%, 400%, 500% production of IFNγ and TNFα in cells treated with such exemplary compounds relative to that of cells treated with negative control (DMSO).

TABLE 10

| | % TNFa + /IFNg + production in compound- | | % TNFa + /IFNg + production in compound- |
|---|---|---|---|
| Compound | treated cells (uM compound) relative to negative | Compound | treated cells (uM compound) relative to negative |
| No. | control (DMSO) | No. | control (DMSO) |
| 113 | 210 | 139 | 226 |
| 117 | 236 | 200 | 227 |
| 120 | 194 | 158 | 152 |
| 187 | 168 | 149 | 207 |
| 151 | 227 | 176 | 193 |
| 135 | 504 | 143 | 174 |
| 183 | 162 | 178 | 168 |
| 204 | 430 | 154 | 152 |
| 150 | 260 | 153 | 132 |
| 144 | 251 | | |

### Example 6: In Vivo Efficacy of PTPN2 Inhibitors in Mouse Tumor Model and Impact on Pharmacodynamic Markers

Tumor Cell Inoculation and Treatments.

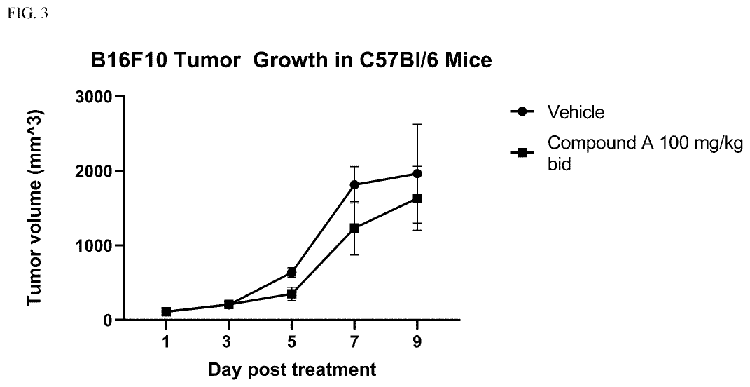

Cells are grown to passage 3 in vitro. A total of 1 to 5×10⁵ viable B16F10 cells are inoculated subcutaneously into the right flank of female C57BL/6 mice (7-12 weeks old) on Day 0. The injection volume is 0.1 mL and is composed of a 1: 1 mixture of S-MEM and Matrigel® (Corning, NY, USA). The mean tumor volume (TV) at randomization is approximately 100-200 mm³. Following randomization, treatments are initiated on the same day. Dosing of mice is conducted orally, twice a day (BID). Mice are dosed (100 mg/kg/dose or 300 mg/kg/dose) with a compound described herein or vehicle controls. Tumor volume is calculated three times weekly. Measurements of the length (L) and width (W) of the tumor are taken via electronic caliper and the volume is calculated according to the following equation: V=L×W²/2. Tumor growth inhibition (TGI) is calculated as TGI=1−(Mean TV _{Timepoint(Treatment)}/Mean_{TV Timepoint (Vehicle)}) for each timepoint that tumor volumes are measured and depicted in FIG. 3. FIG. 3 shows that apparent tumor stasis and shrinkage is observed in animals dosed with compound A.

pSTAT5 Flow Cytometry Assay in Mouse Whole Blood.

The same mouse model is utilized to assess pSTAT5 expression enhancement when treated with compound A relative to vehicle as the negative control. Whole blood is drawn into EDTA powder coated tubes by cardiac puncture from mice on day 3 of dosing with a compound described herein (2 hours after the 6^{th} dose). 90 μL of whole blood is stimulated with 10 μL of murine IL-2 to achieve a final concentration of 100 ng IL-2/mL (R&D Systems, Minneapolis, MN, cat #402-ML) for 20 minutes at 37° C., 5% CO₂. After stimulation, 1.8 mL of prewarmed BD Phosflow Lyse/Fix Buffer (BD Biosciences, San Jose, CA) is added for 20 minutes at 37° C. Cells are washed twice in FACS buffer (Dulbecco's PBS with 0.2% BSA) and incubated for 30 minutes on ice in cold Perm Buffer III (BD Biosciences, San Jose, CA). Cells are washed with FACS buffer and resuspended in 50 μL of FACS buffer with antibodies and stained for 3 hours at room temperature with gentle shaking. The antibodies added are a combination of the following: anti-CD3-AF647, clone 145-2C11 (Biolegend, Cat #564279); anti-CD4-FITC, clone GK1.5 (Biolegend, San Diego, CA, Cat #100406); anti-pSTAT5 (pY694)-PE, clone 47 (BD Biosciences, San Jose, CA, Cat #562077); anti-CD45-BUV395, clone 30-Fl1 (BD Biosciences, San Jose, CA, cat #564279). After staining, cells are washed twice with FACS buffer, and the samples are acquired on a Ze5 flow cytometer (Biorad, Hercules, CA) and analyzed with FLowJo V10 software (FlowJo, Ashland, OR). The mean fluorescence intensity (MFI) of pSTAT5 as a measure of the amount of phosphorylated STAT5 in the CD3+ T cell population from vehicle or compound treated animals is reported with and without IL-2 stimulation. One or more compounds disclosed herein exhibit the ability to augment pSTAT5 level in response to IL-2 stimulation.

Tumor Infiltrating Lymphocyte Exhaustion Assay

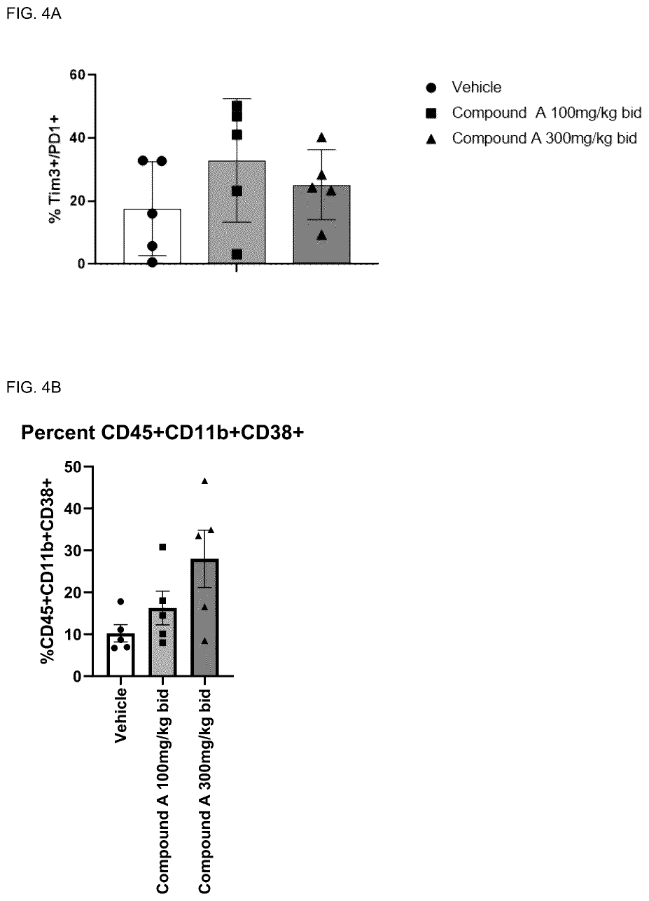

The same mouse model is utilized to assess the amount of tumor infiltrating lymphocytes when treated with or without an exemplary compound A of the present disclosure. C57BL/6 mice were implanted with 5*10⁵ B16F10 cells formulated in Matrigel via the subcutaneous route. After tumors reached a size of 50-150 mm³, mice were randomized. 7 days post randomization, tumor cells were processed on a GentleMACS octo dissociator and stained for exhaustion markers using a panel of antibodies: Live/Dead, CD45.1, CD8, TIM-3, PD-1, SLAMF6, CD11b, CD38. In parallel, splenocytes were stained with the same cocktail of antibodies and analyzed by flow cytometry. These results indicated that while peripheral splenic T cells were mostly naïve and unactivated, tumor infiltrating T cells bore an exhausted phenotype. PTPN2 inhibitors are tested in the described TIL exhaustion assay to measure modulation of exhaustion in the tumor microenvironment. FIG. 4 shows measurement of control cells. Day 7 post dosing shows an increase in both TIM3+/PD1+ T cells with compound A, as well as an increase in CD38+/CD11b+ “M1” Macrophages.

### Example 7: Cytokine Release Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor for 12-24 hours (optionally longer). The activated effector cells are induced to release cytokine by co-culturing with target cells at a desired Effector:Target cell ratio, e.g., 10:1, 5:1, or 1:1. Co-culture supernatant is harvested after approximately 20 hrs. These supernatants are then used to measure the released cytokines such as IL-2 and IFN-γ, using Meso Scale Discovery, Proinflammatory Panel 1 catalog #N05049A-1 system according to the manufacturer's protocol. The target cells can be irradiated (e.g. at 10,000) prior to co-culturing with the effector cells. The ability of a PTPN2 inhibitor to induce an increase in cytokine release (e.g., IL-2 or IFN-γ) by T cells in response to the antigen to which the TFP or CAR binds can be demonstrated by this example.

### Example 8: Cell Proliferation Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor disclosed herein for 12-24 hours (optionally longer). The activated effector cells are induced to proliferate by co-culturing with target cells that comprise the target tumor antigen. Typically the target cells are irradiated, washed and counted. Co-culturing is performed at a desired Effector: Target cell ratio, e.g., 10:1, 5:1, or 1:1. Proliferation of the effector cells are evaluated, typically after bead expansion for about 10 days. The number of cells per mL and the viability of cells are measured by Cellometer. This example can be performed to test or demonstrate the ability of a PTPN2 inhibitor (e.g., compound described herein) to potentiate effector cell number and viability relative to effector cells not treated with a PTPN2 inhibitor (e.g., compound described herein).

### Example 9: Cytotoxicity Assay

Effector cells (e.g., non-modified T cells, or TFP- or CAR-expressing T cells) are activated with CD3/CD28 beads (CD3/CD28 CTS Dynabeads) in the presence of a PTPN2 inhibitor (e.g., compound described herein) for 12-24 hours (optionally longer). Target cells (e.g., cancer or tumor cells) that comprise the target tumor antigen to which the TFP or CAR binds are incubated with Calcein-AM in the dark, washed, and counted. The activated effector cells are co-cultured with the target cells. Co-culturing is performed at a desired Effector: Target cell ratio, e.g., 10:1, 5:1, or 1:1. At the end of the co-culture period (e.g., 5 hours), the number of target cells and the viability of the target cells are assessed by measuring Calcein fluorescence from the collected cells. This example is to demonstrate that PTPN2 inhibition by a PTPN2 inhibitor (e.g., compound described herein) yields an increase in cytotoxicity of effector cells against target cells relative to effector cells not treated with a PTPN2 inhibitor (e.g., compound described herein). This example can be performed to test or demonstrate the ability of a PTPN2 inhibitor (e.g., compound described herein) to potentiate effector cell number and viability relative to effector cells not treated with a PTPN2 inhibitor (e.g., compound described herein).

### Example 10: CAR-T Killing Assay

The ability of PTPN2 inhibitors to potentiate tumor cell killing using CAR-Ts that express a tumor antigen is demonstrated as follows. Specific CAR-T cells (CAR-Ts) are generated by transducing primary human CD3+ T cells (Discovery Life Sciences) with a lentivirus expressing a chimeric antigen receptor specific to antigen, as well as GFP (Creative Bio). Prior to transduction, T cells are stimulated overnight with anti-CD3 and anti-CD28 antibodies coated onto magnetic beads (Invitrogen) at a 1:1 bead-to-cell ratio. Four days after transduction, the beads are removed and the following day CAR-Ts are sorted based on GFP expression and expanded in hIL7 (10 ng/mL) (Peprotech) and hIL15 (5 ng/mL) (Peprotech) for an additional 8 days. Thereupon, CAR-Ts are co-cultured with a Nuclight Red (Essen Biosciences)-labeled antigen positive tumor line or an antigen negative line for 18h at a 1:1 effector:target cell ratio.

CAR-Ts are pretreated with 0.1% DMSO (vehicle control) or a PTPN2 inhibitor (e.g., compound described herein) for 1-2.5 hours prior to co-culture with cell lines at indicated concentrations. PTPN2 inhibitor (e.g., compound described herein) is washed from CAR-Ts prior to inclusion in the tumor killing assay. Tumor killing are assessed by comparing DMSO-treated CAR-Ts to PTPN2 inhibitor-treated CAR-Ts using either flow cytometry or Incucyte imaged-based assessment of tumor cell viability, at various effector:target ratios. Percent killing is assessed by calculating the amount of viable cells at a given time point as compared to untreated tumor cells, tumor cells treated with PTPN2 inhibitor (e.g., compound described herein), and tumor cells cultured with DMSO treated CAR-T cells as a control. This example can be performed to test or demonstrate the ability of a PTPN2 inhibitor (e.g., compound described herein) to potentiate CAR-T cells' tumor killing activity.

### Example 11: CAR-T Adoptive Cell Transfer Xenograft Tumor Assay

For in vivo studies with CAR-Ts, nude mice are implanted with OVCAR xenografts. After reaching a suitable size of 50-100 mm³, approximately 10⁶ CAR-Ts transiently treated (e.g., for 1 hr and then washing away) with or without PTPN2 inhibitor (e.g., compound described herein) are transferred i.v. into tumor bearing mice. Tumor volume and CAR-T cell count are measured at multiple time points and compared to control groups treated with DMSO (e.g., for 1 hr and then washing away). This example can be performed to test or demonstrate the ability of a PTPN2 inhibitor (e.g., compound described herein) to lower tumor volume, higher frequency of CAR-Ts in the blood, and/or greater infiltration and/or activation of CAR-Ts into tumors, smaller tumor volume and/or higher CAR-T cell counts.

### Example 12: Mouse Adoptive Cell Transfer Syngeneic Tumor Assay

Thy1.1 congenic C57BL/6 mice are implanted subcutaneously with approximately 5×10⁵ of OVA expressing syngeneic tumor cells (B16-OVA, EL4 OVA, or YUMM1.1) formulated with 50% Matrigel (50% PBS). Prior to the implantation, the B16-OVA, EL4 OVA, or YUMM1.1 tumor cell lines are transduced with a lentivirus encoding an OVA-GFP fusion protein. After sorting for GFP expression, the B16-OVA, EL4 OVA, or YUMM1.1 cells are shown to grow in untreated C57BL/6 mice. After growing to a volume of ˜50-100 mm³, tumor bearing mice would receive i.v. transfer of approximately 1×10⁶ OT-1 transgenic T cells that will undergo the following treatments. First, primary OT-1 splenocytes are treated with 10 nm of SIINFEKL peptide or anti-CD3/anti-CD28 coated beads. After 2 days, the cells are washed and transferred into culture medium with IL-2, IL-7 and IL-15 (all at 5 ng/ml) for another 3 days. In other experiments, naïve OT-1 CD8 T cells are isolated for transfer. Prior to the transfer, the OT-1 cells are treated with DMSO (vehicle control) or a PTPN2 inhibitor (e.g., compound described herein) for 1 hour and washed in PBS two times prior to injection.

### Example 13: Mobility Shift Assay to Assess Direct Binding to PTPN2 by Compounds Disclosed Herein and/or Associated Potency

Protein Expression and Purification:

PTPN2 Iso 1-314 plasmid was synthesized by ATUM, in to pD441-SR: T5-sRBS-ORF, Ecoli-Elec D vector.

The DNA sequence is shown below (SEQ ID NO. 2):

ATGCACCATCATCACCATCACGAAAATCTGTATTTCCAAGGTATGCCTA
CTACTATTGAACGTGAATTTGAAGAACTGG
ATACTCAACGTCGTTGGCAGCCGCTGTATTTAGAGATCCGTAATGAAAG
CCATGACTATCCGCACAGAGTGGCGAAATT
CCCGGAGAATCGTAACCGCAATCGTTATCGCGACGTTTCTCCGTACGAC
CACAGCCGCGTCAAGCTCCAGAACGCCGA
GAACGACTACATCAACGCGTCCCTGGTCGACATCGAAGAGGCACAGCGC
TCTTACATCCTGACCCAGGGTCCGCTGCC
GAATACCTGTTGTCATTTCTGGTTGATGGTCTGGCAGCAGAAAACGAAA
GCTGTTGTGATGCTGAATCGTATCGTGGAG
AAAGAATCCGTAAAGTGTGCCCAATACTGGCCGACCGATGATCAAGAAA
TGCTGTTCAAAGAAACCGGTTTTAGCGTT
AAGCTGCTGAGCGAAGATGTGAAAAGCTACTATACGGTGCATCTGCTGC
AACTGGAGAACATTAACAGCGGCGAAACC
CGTACCATCAGCCACTTTCACTACACCACGTGGCCAGACTTTGGTGTTC
CGGAGTCGCCGGCAAGCTTCCTTAACTTTCT
GTTCAAGGTTCGCGAGTCCGGTAGCCTGAACCCGGATCATGGTCCGGCA
GTTATCCACTGCAGCGCTGGCATTGGCCGT
AGCGGTACCTTTAGCTTGGTCGACACGTGCCTGGTGCTGATGGAAAAGG
GTGACGACATTAACATTAAACAAGTTCTGC
TGAATATGCGCAAATATCGCATGGGTCTGATCCAGACGCCGGATCAGCT
GCGTTTCTCTTACATGGCGATTATTGAGGG
CGCCAAATGCATTAAGGGCGATAGCAGCATCCAGAAGCGTTGGAAAGAA
CTGAGCAAGGAAGATTTGAGCCCTGCGTT
CGATCACTCCCCAAATAAGATCATGACCGAGAAGTATAACTAA

Protein amino acid sequence is shown below (SEQ ID NO. 3):

MHHHHHHENLYFQGMPTTIEREFEELDTQRRWQPLYLEIRNESHDYPHR
VAKFPENRNRNRYRDVSPYDHSRVKLQNAENDYINASLVDIEEAQRSYI
LTQGPLPNTCCHFWLMVWQQKTKAVVMLNRIVEKESVKCAQYWPTDDQE
MLFKETGFSVKLLSEDVKSYYTVHLLQLENINSGETRTISHFHYTTWPD
FGVPESPASFLNFLFKVRESGSLNPDHGPAVIHCSAGIGRSGTFSLVDT
CLVLMEKGDDINIKQVLLNMRKYRMGLIQTPDQLRFSYMAIIEGAKCIK
GDSSIQKRWKELSKEDLSPAFDHSPNKIMTEKYN

BL21(DE3) Star (ThermoFisher) competent cells were transformed with the plasmid following exactly the manufacturer's protocols. Single colonies from LB Agar Kanamycin plates were picked and inoculated in 50 mL LB media supplemented with 0.05 g/mL Kanamycin. The culture was incubated at 37° C. with shaking for 20 hours before transferring to a larger flask containing 1 L of terrific broth media. 1 L culture was incubated at 37° C. with shaking until optical density of the media reached 0.3. The temperature was lowered to 18° C. and cells were grown at the temperature until optical density reached 0.6. Isopropyl β-D-1-thiogalactopyranoside(IPTG) was added to the media to a final concentration of 0.2 μM. The media was then induced for 20 hours at 18° C. with shaking.

Cells were harvested with 4000 rpm centrifugation, resuspended in buffer containing 20 mM Tris HCl pH 8.0, 10% glycerol, 0.1% β-mercaptoethanol, 40 mg of hen egg white lysozyme, and 1 dissolved tablet of protease inhibitor cocktail. Resuspended cells were lysed with sonication using 10 second on and 20 second off time at 40% power level with constant stirring on ice for 20 minutes. The lysate was centrifuged for 10 minutes at 14500 rpm to remove the insoluble fractions. The soluble fraction of the lysate was then incubated with 2 mL of cobalt resin pre-equilibrated with 20 mM Tris HCl, pH 8.0 10% glycerol and 0.1% I-mercaptoethanol buffer for 1 hour in a 4° C. cold room. The resin was then collected in a filtered column and washed with 10 mL of buffer containing 20 mM Tris HCl pH 8.0, 10% glycerol, 0.1% β-mercaptoethanol, and 10 mM imidazole. Then the bound PTPN2 protein was eluted with buffer containing 20 mM Tris HCl pH 8.0, 10% glycerol. 0.1% β-mercaptoethanol, and 500 mM imidazole.

The eluted protein was then injected onto an Q Sepharose® anion exchange column with equilibration buffer containing 20 mM Tris HCl pH 8.0, 5% glycerol, 2 mM DTT and elution buffer containing 20 mM Tris HCl pH 8.0, 500 mM NaCl, 0.5% glycerol, 2 mM DTT as the elution buffer. The protein was eluted with a linear gradient of the two buffers. The collected protein was checked with SDS-PAGE and fractions containing PTPN2 were pooled and further purified with a Superdex® 200 size exclusion column in buffer containing 10 mM Tris HCl pH8.5, 150 mM NaCl, and 2 mM DTT.

Thermal Shift Assay

Protein in 10 mM Tris HCl pH 8.5, 150 mM NaCl, and 2 mM DTT was adjusted to a concentration of 1 mg/mL(26 uM) in the same buffer. Diluted protein was incubated overnight with 200 uM compound at 4° C. Sample volume in each well is 20 μL consisting of 18 μL of the protein solution and 2 μL of the SyproOrange dye (50×) diluted in buffer containing 10 mM Tris HCl pH 8.5, 150 mM NaCl, and 2 mM DTT in a 384 PCR plate. The plate was sealed with optically clear plate seal. Thermal shift experiment was run on a Q-PCR machine. Thermal cycle used was a linear gradient form 25° C. to 100° C. and a 2.4° C./minute linear gradient.

Thermal Shift Assay Results

Thermal shift assay results directly demonstrated the binding and stabilizing effects of selected PTPN2 inhibitors. The binding of PTPN2 inhibitors shifts the melting curves of the protein and the level of stabilization was measured by the difference of the melting temperature. The compounds disclosed herein (e.g., compound nos. 175, 143, 178, 186, 180, 154, 153, 136, 133, 185) confer different levels of thermal stabilization to the PTPN2 protein reflected in the melting temperature shifts ranging from at least 2.0° C., 3.0° C., 4.0° C., 5.0° C., 6.0° C., 7.0° C., 8.0° C., 9.0° C., 10.0° C. or even higher.

### Example 14: Mouse Oral Exposure (% F)

Pharmacokinetic Profile for Test Compound is Measured by Oral Dosing of Female CD-1 Mice.

Animal weights are typically between 20-50 grams, and animals are allowed to acclimate to their new environment for at least 3 days prior to the initiation of any studies. One set of animals is dosed oral (po) with test compound, 10 mg/kg in 20% HP-beta-CD or 20% Captisol, pH adjusted to ˜4 by citric acid. Oral dosing solution concentration is 1 mg/ml test compound. Time of blood sampling is 15 minutes, 30 minutes, 60 minutes, 120 minutes, 240 minutes, 480 minutes, and 24 hours following oral (po) dosing. Another set of animals is dosed intravenous (iv) using a 0.2 mg/ml test compound dosing solution concentration. Blood samples (˜0.2 mL/sample) is collected via cardiac puncture or submandibular bleed and placed in tubes containing EDTA-K2 and stored on ice until centrifuged. The blood samples are centrifuged at approximately 6800 g for 6 minutes at 2-8° C. and the resulting plasma will be separated and stored frozen at approximately −80° C.

All the plasma samples are analyzed using an Agilent Technologies 6430 Triple Quad LC/MS system, following the manufacture instructions. All the analytes are detected with positive-mode electrospray ionization (ES+). A standard curve for each test compound is generated and used to measure test compound concentrations in the mouse plasma samples. Based on the time course sampling, an area under the curve is calculated for the oral dose group and the intravenous dose group. Percentage mouse bioavailability is calculated based on the equation:

$$\% F ( mouse ) = \frac{{AUC}_{po} * {Dose}_{IV}}{{AUC}_{IV} * {Dose}_{po}} ,$$

where F is bioavailability, AUCₚₒ is area under curve of oral drug, AUC_{IV} is area under curve of intravenous drug, Dose_{IV} is the intravenous dose and Doseₚₒ is the oral dose.

### Example 15: Rat Oral Exposure (% F)

Pharmacokinetic profile for test compound is measured by oral dosing of male Sprague-Dawley rats.

Animal weights are typically over 200 grams, and animals are allowed to acclimate to their new environment for at least 3 days prior to the initiation of any studies. One set of animals is dosed oral (po) with test compound, 10 mg/kg in 20% HP-beta-CD or 20% Captisol, pH adjusted to ˜4 by citric acid. Oral dosing solution concentration is 1 mg/ml test compound. Time of blood sampling is 15 minutes, 30 minutes, 90 minutes, 180 minutes, 360 minutes and 24 hours following oral (po) dosing. Another set of animals is dosed intravenous (iv) using a 0.4 mg/ml test compound dosing solution concentration. Blood samples (˜0.2 mL/sample) is collected via jugular vein and placed in tubes containing EDTA-K2 and stored on ice until centrifuged. The blood samples are centrifuged at approximately 6800 g for 6 minutes at 2-8° C. and the resulting plasma will be separated and stored frozen at approximately −80° C.

All the plasma samples are analyzed using an Agilent Technologies 6430 Triple Quad LC/MS system, following the manufacture instructions. All the analytes are detected with positive-mode electrospray ionization (ES+). A standard curve for each test compound is generated and used to measure test compound concentrations in the rat plasma samples. Based on the time course sampling, an area under the curve is calculated for the oral dose group and the intravenous dose group. Percentage rat bioavailability is calculated based on the following equation:

$$\% F ( rat ) = \frac{{AUC}_{po} * {Dose}_{IV}}{{AUC}_{IV} * {Dose}_{po}} ,$$

where F is bioavailability, AUCₚₒ is area under curve of oral drug, AUC_{IV} is area under curve of intravenous drug, Dose_{IV} is the intravenous dose and Doseₚₒ is the oral dose

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention. It is intended that the following claims define the scope of the invention and that methods and structures within the scope of these claims and their equivalents be covered thereby.

1. A compound of Formula (I-3), or a pharmaceutically acceptable salt or solvate thereof: 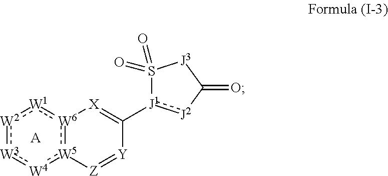
wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R^{2a}), C(O), S(O), S(O)₂, S(O)(NR²), S(O)(R²), or P(O)(R²);
W³ is N, N(R³), C(R³), C(R³)(R^{3a}), C(O), S(O), S(O)₂, S(O)(NR³), S(O)(R³), or P(O)(R³);
W⁴ is N, N(R⁴), C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein at least one of W, W², W³, W⁴, W¹, and W⁶ is N, N(R¹), N(R²), N(R³), or N(R⁴); and
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰);
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵¹, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵¹, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵¹, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₆cycloalkyl, C₁₋₆ heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}), wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆ cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉ heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₁₋₆aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆ heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl; [symbol]
indicates a single or double bond such that all valences are satisfied; and
provided that:
at least two of Ring A [symbol] double bonds.

2. The compound of claim 1, or a pharmaceutically acceptable salt or solvate thereof, wherein X is C(R⁵), Y is C(R⁶), and Z is C(R⁷).

3-11. (canceled)

12. The compound of claim 1, or a pharmaceutically acceptable salt or solvate thereof, having the structure of Formula (Ia-3):

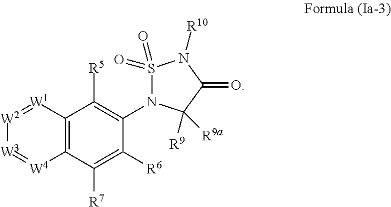

13. The compound of claim 12, or a pharmaceutically acceptable salt or solvate thereof, wherein W¹ is N, W² is C(R²), W³ is C(R³), and W⁴ is C(R⁴).

14. The compound of claim 12, or a pharmaceutically acceptable salt or solvate thereof, wherein R³ is hydrogen, and R⁴ is hydrogen.

15-20. (canceled)

21. A compound of Formula (III-3), or a pharmaceutically acceptable salt or solvate thereof: 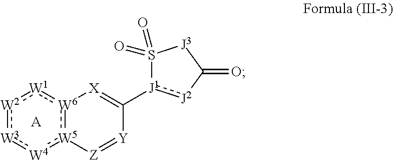
wherein:
W¹ is N, N(R¹), C(R¹), C(R¹)(R^{1a}), C(O), S(O), S(O)₂, S(O)(NR¹), S(O)(R¹), or P(O)(R¹);
W² is N, N(R²), C(R²), C(R²)(R^{2a}), C(O), S(O), S(O)₂, S(O)(NR²), S(O)(R²), or P(O)(R²);
W³ is N, N(R³), C(R³), C(R³)(R^{3a}), C(O), S(O), S(O)₂, S(O)(NR³), S(O)(R³), or P(O)(R³);
W⁴ is N, N(R⁴), C(R⁴), C(R⁴)(R^{4a}), C(O), S(O), S(O)₂, S(O)(NR⁴), S(O)(R⁴), or P(O)(R⁴);
W⁵ is N or C;
W⁶ is N or C; wherein
i) at least one of W¹, W², W³, W⁴, W⁵, and W⁶ is N, N(R¹), N(R²), N(R³), N(R⁴), or O; and
ii) at least one of W¹, W², W³, and W⁴ is C(O), S(O), S(O)₂, S(O)(NR¹), S(O)(R¹), P(O)(R¹), S(O)(NR²), S(O)(R²), P(O)(R²), S(O)(NR³), S(O)(R³), P(O)(R³), S(O)(NR⁴), S(O)(R⁴), or P(O)(R⁴);
X is N or C(R⁵);
Y is N or C(R⁶);
Z is N or C(R⁷);
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ and R^{1a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or R¹ and R^{1a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20a};
R² and R^{2a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R² and R^{2a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20b};
R³ and R^{3a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; or R³ and R^{3a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20c};
R⁴ and R^{4a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d}; or R⁴ and R^{4a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
R⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)RD, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20k};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₆cycloalkyl, C₁₋₆ heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20k};
each R¹⁶ is independently selected from —C₁₋₆alkylene-OP(O)(OR^{16a})(OR^{16b}) and —P(O)(OR^{16a})(OR^{16b}), wherein the C₁₋₆alkylene is optionally substituted with one, two, or three R^{20l};
each R^{16a} and R^{16b} is independently selected from hydrogen, C₁₋₆alkyl, C₂₋₆alkenyl, C₂₋₆alkynyl, C₃₋₆ cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉ heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20m};
each R^{20a}, R^{20b}, R^{20c}, R^{20d}, R^{20e}, R^{20f}, R^{20g}, R^{20h}, R²⁰ⁱ, R^{20j}, R^{20k}, R^{20l}, and R^{20m} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl; [symbol]
indicates a single or double bond such that all valences are satisfied.

22. (canceled)

23. The compound of claim 1, or a pharmaceutically acceptable salt or solvate thereof, wherein R⁵ is selected from hydrogen and halogen.

24. The compound of claim 1, or a pharmaceutically acceptable salt or solvate thereof, wherein R⁶ is —OH.

25. The compound of claim 1, or a pharmaceutically acceptable salt or solvate thereof, wherein R⁷ is hydrogen.

26. The compound of claim 1, or a pharmaceutically acceptable salt or solvate thereof, wherein R⁹ is hydrogen, R^{9a} is hydrogen, and R¹⁰ is hydrogen.

27. The compound of claim 1, or a pharmaceutically acceptable salt or solvate thereof, wherein:
R² is —OR¹²; and
R¹² is independently selected from C₁₋₆alkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20k}.

28. (canceled)

29. The compound of claim 1, or a pharmaceutically acceptable salt or solvate thereof, wherein R² is selected from

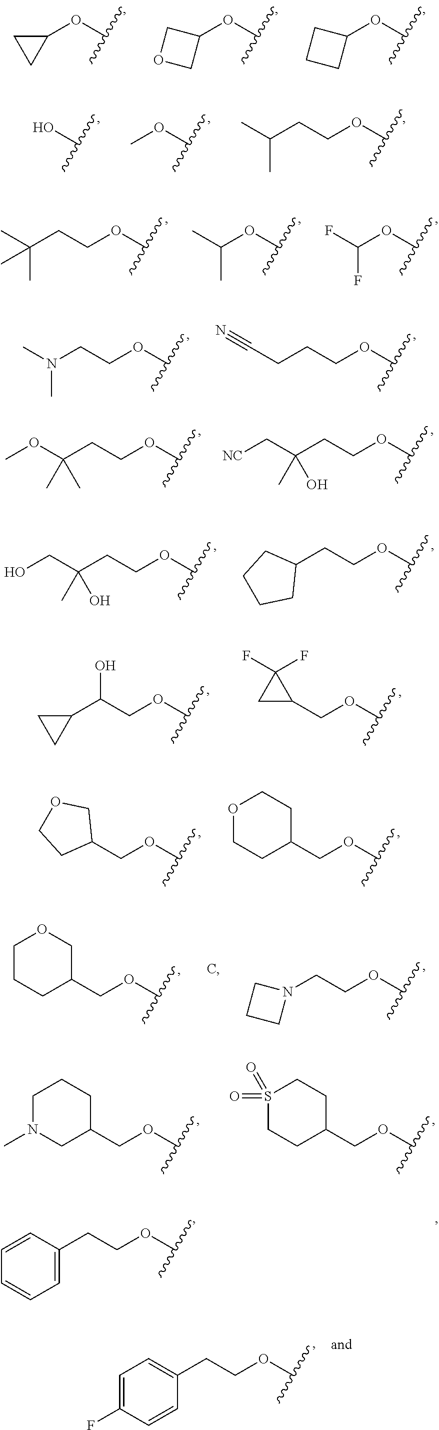

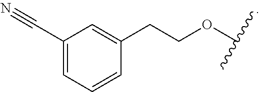 [SMILES: *OCCc1cccc(C#N)c1]

30. The compound of claim 1, or a pharmaceutically acceptable salt or solvate thereof, wherein:
R² is —N(R¹²)(R¹³);
R¹³ is independently hydrogen; and
R¹² is independently selected from C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, wherein C₁₋₆alkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, are optionally substituted with one, two, or three R^{20k}.

31. (canceled)

32. The compound of claim 1, or a pharmaceutically acceptable salt or solvate thereof, wherein R² is selected from

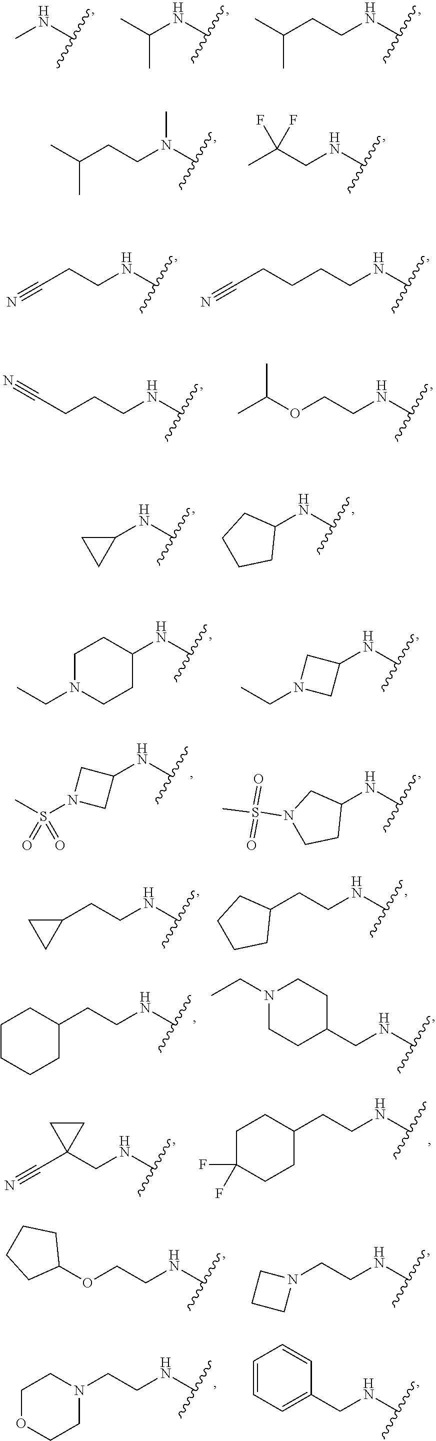

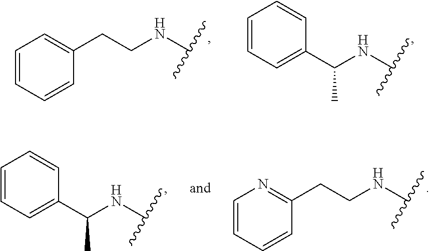

33-35. (canceled)

36. The compound of claim 1, or a pharmaceutically acceptable salt or solvate thereof, wherein R² is selected from C₁₋₆alkyl, C₂₋₉heterocycloalkyl, C₁₋₉ heteroaryl, —OR¹², —N(R¹²)(R¹³), —C(O)R¹⁵, and —C(O)N(R¹²)(R¹³), wherein C₁₋₆alkyl, C₂₋₉heterocycloalkyl, and C₁₋₉heteroaryl are optionally substituted with one, two, or three R^{20b}.

37-40. (canceled)

41. The compound of claim 1, or a pharmaceutically acceptable salt or solvate thereof, wherein R³ is selected from hydrogen, halogen, —CN, C₁₋₆alkyl, C₃₋₆cycloalkyl, C₂₋₉heterocycloalkyl, —OR¹², —SR¹², and —N(R¹²)(R¹³), wherein C₁₋₆alkyl, C₃₋₆cycloalkyl, and C₂₋₉heterocycloalkyl are optionally substituted with one, two, or three R^{20c}.

42. (canceled)

43. A compound, or a pharmaceutically acceptable salt or solvate thereof, selected from:

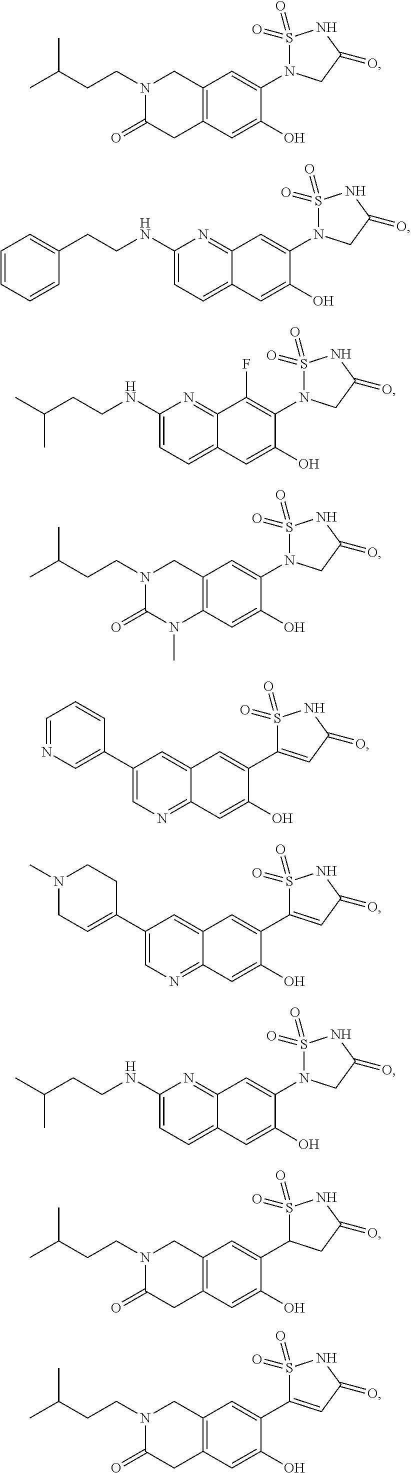

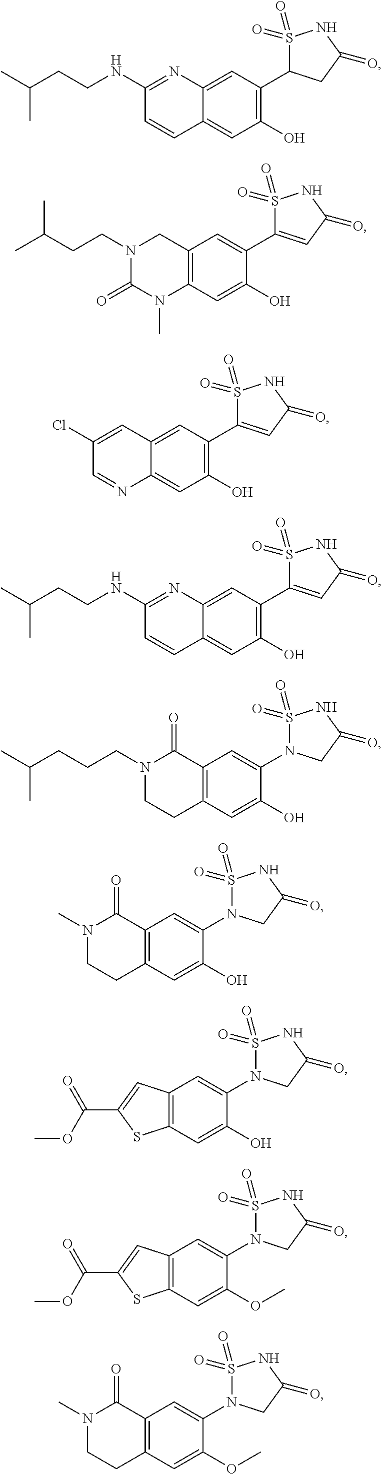

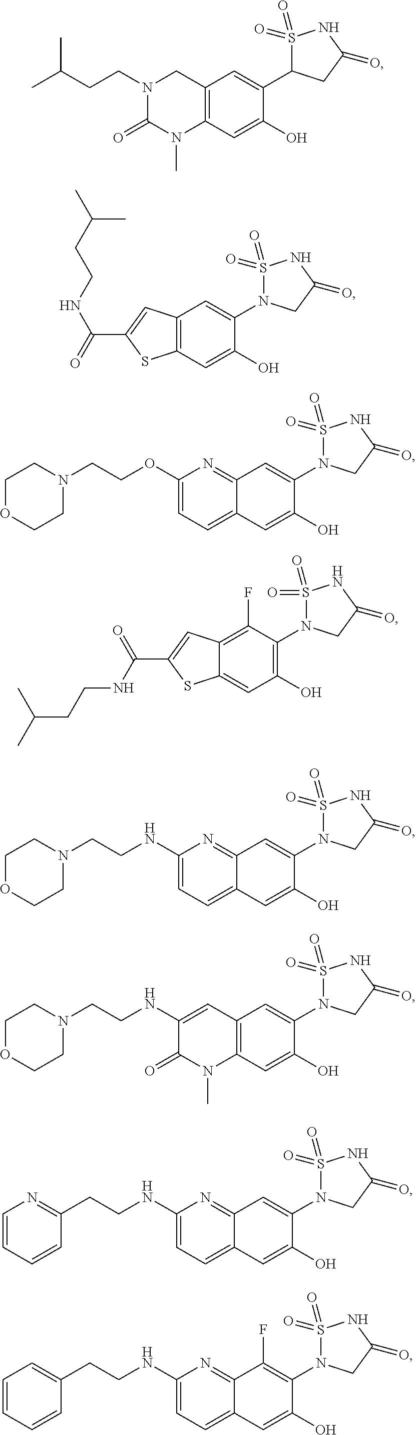

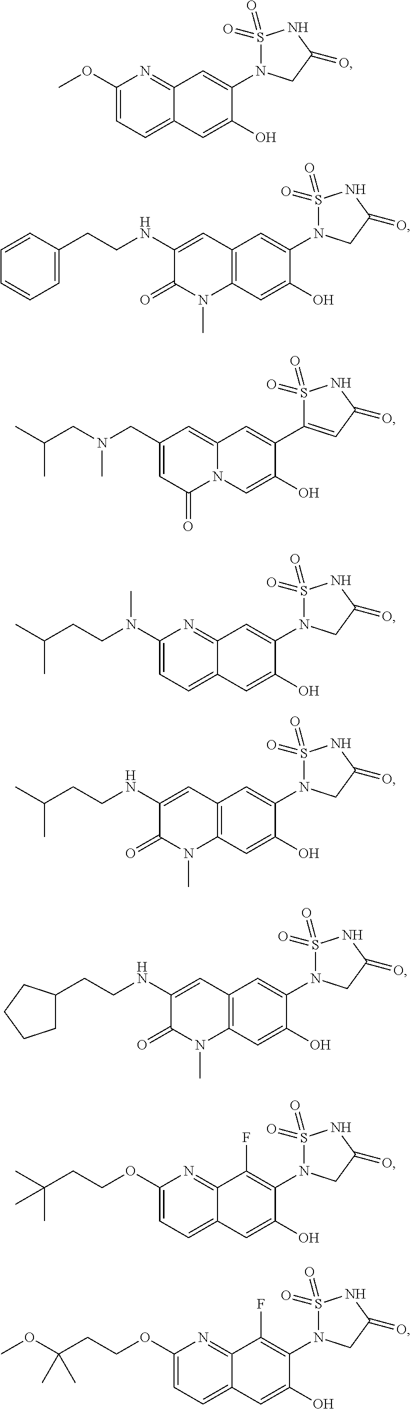

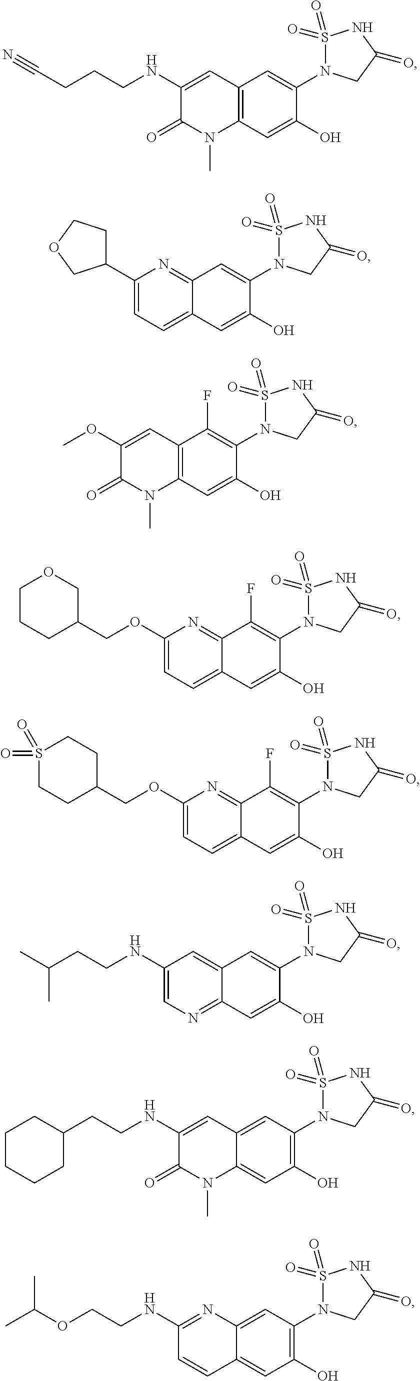

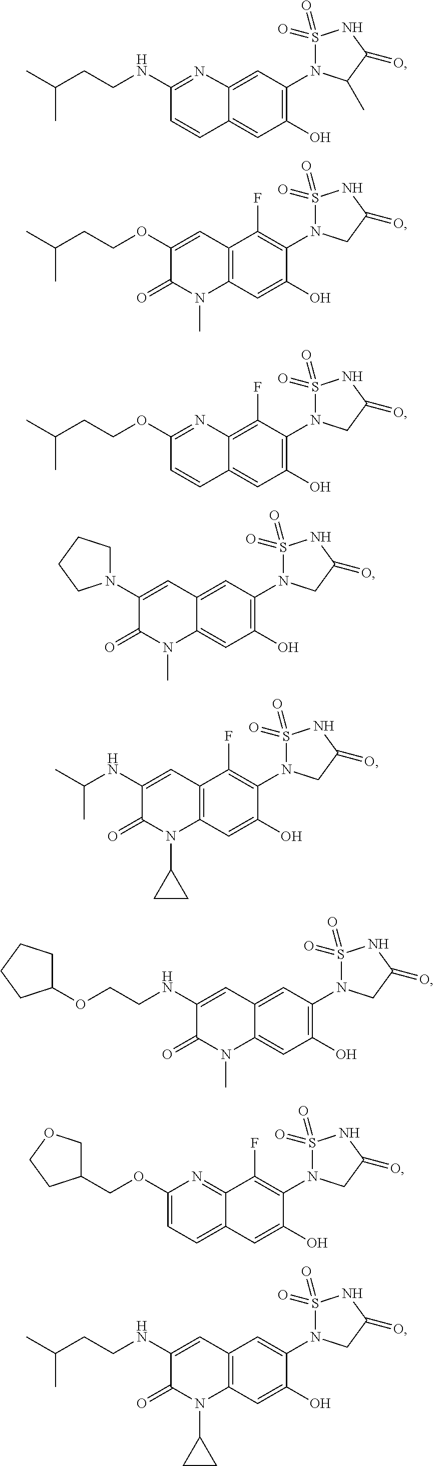

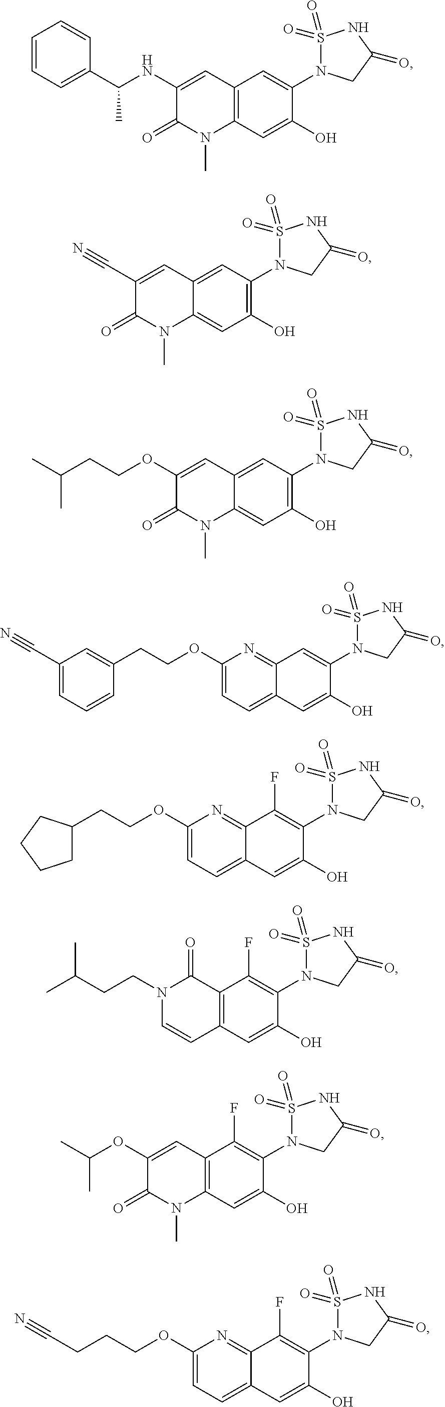

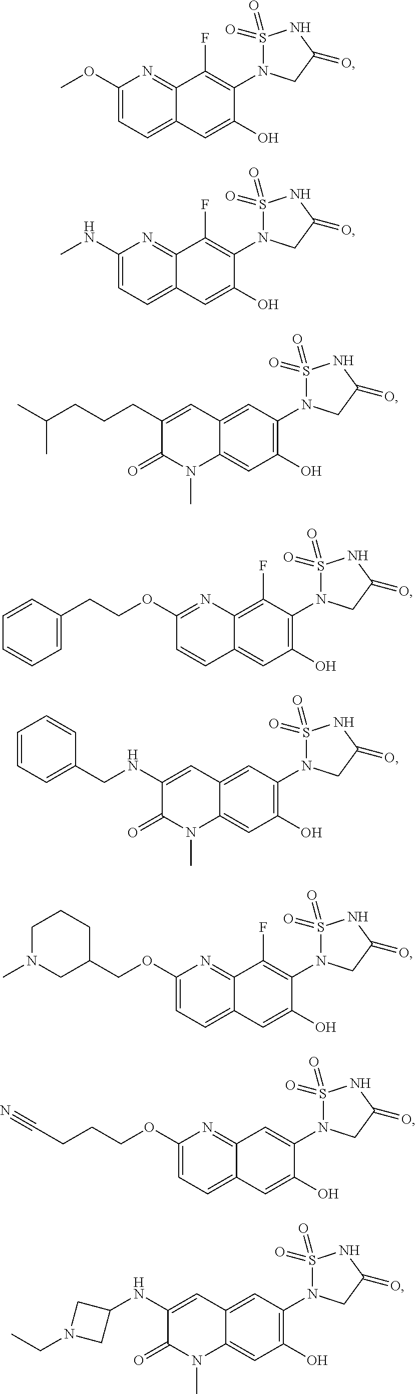

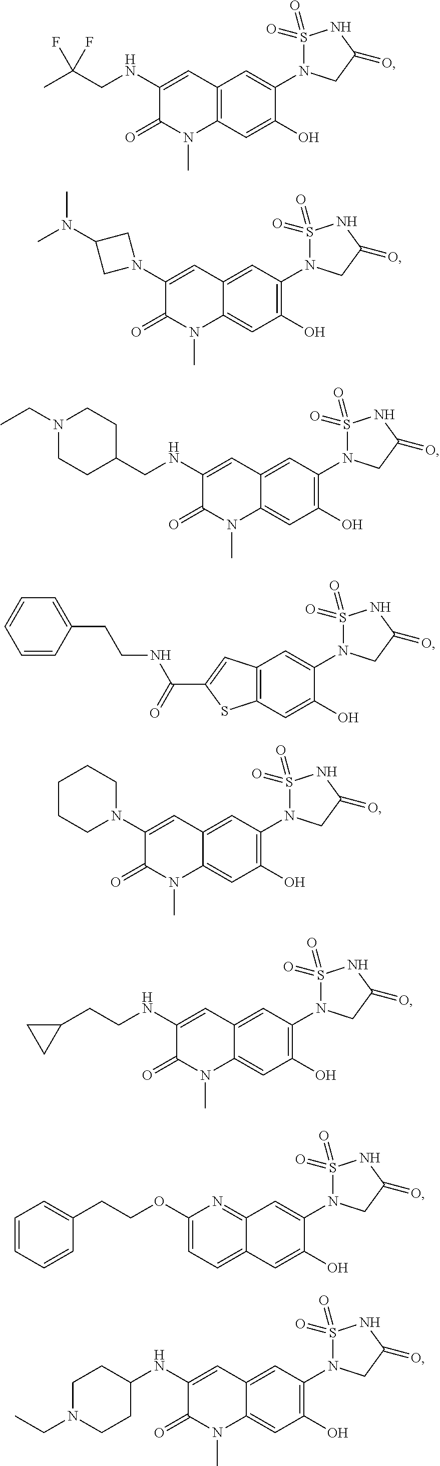

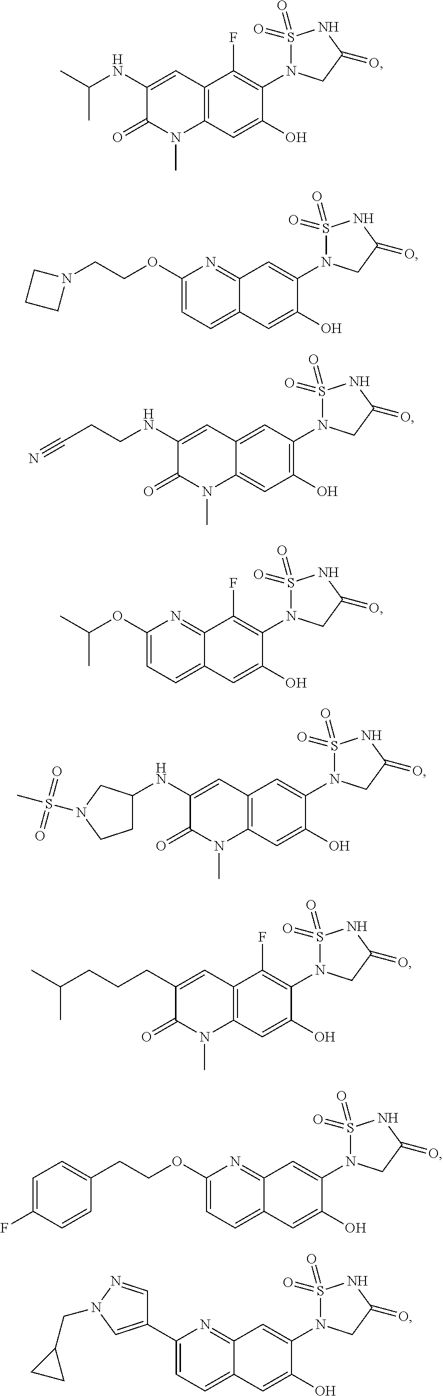

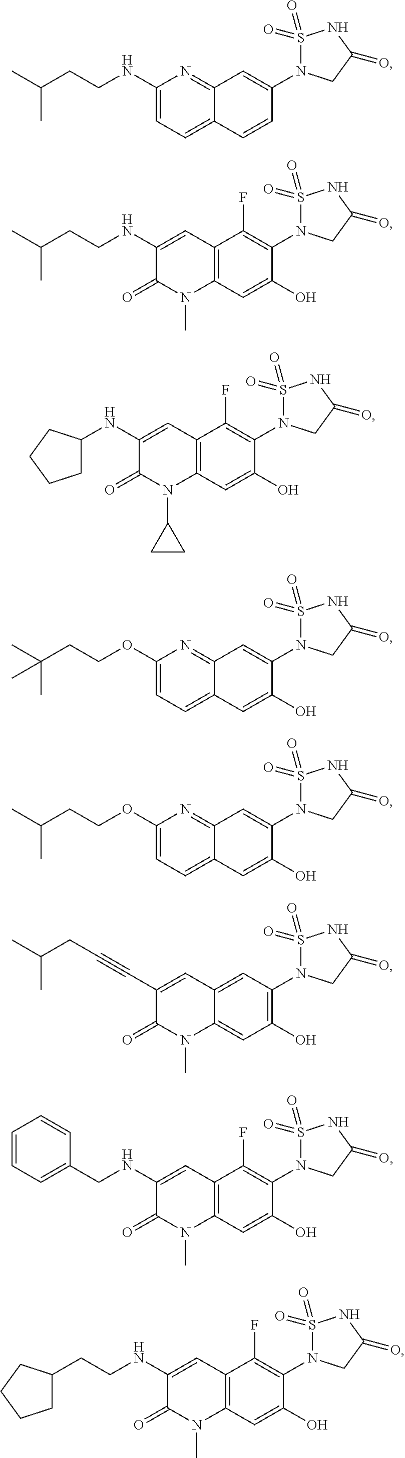

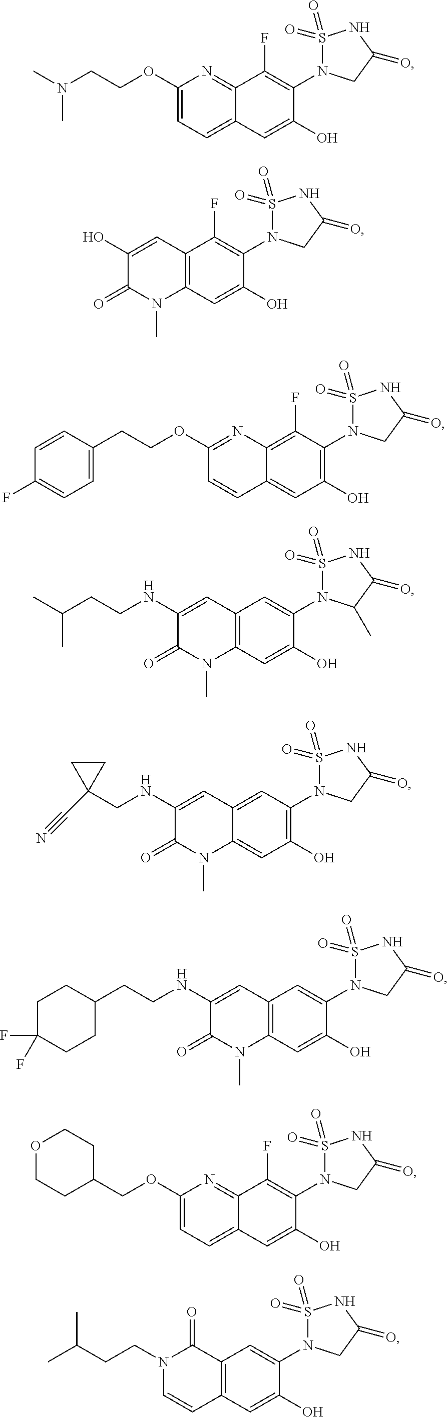

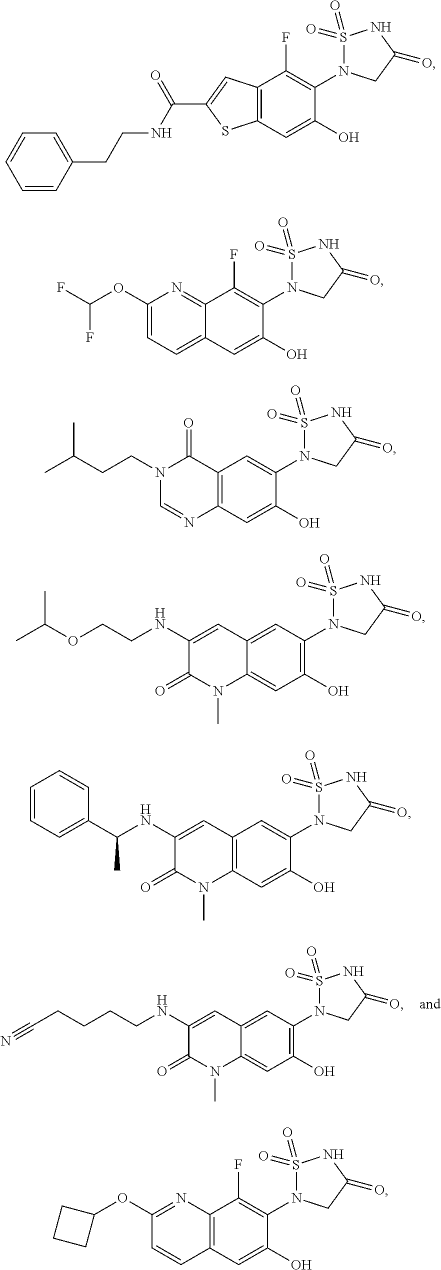

44. A pharmaceutical composition comprising a compound of claim 1, or a pharmaceutically acceptable salt or solvate thereof, and a pharmaceutically acceptable excipient.

45-51. (canceled)

52. A method of potentiating immunity of a subject in need thereof, comprising:
(a) selecting the subject that exhibits expression or activity of PTPN2; and
(b) downregulating expression or activity of PTPN2 by introducing a compound of claim 1 to a cell of the subject, thereby potentiating immunity of the subject.

53-56. (canceled)

57. A method of treating tumor or cancer of a subject in need thereof comprising administering to said subject an effective amount of a compound of claim 1 in conjunction with a cell therapy.


SEQUENCE LISTING (3 sequences)

SEQ ID NO: 1 - protein, 10 aa, synthetic construct
GGGGSGGGGS

SEQ ID NO: 2 - DNA, 987 bp, synthetic construct
  source 1..987: mol_type = other DNA
atgcaccatcatcaccatcacgaaaatctgtatttccaaggtatgcctactactattgaacgtgaatttgaagaactggatactcaacgtcgttggcagccgctgtatttagagatccgtaatgaaagccatgactatccgcacagagtggcgaaattcccggagaatcgtaaccgcaatcgttatcgcgacgtttctccgtacgaccacagccgcgtcaagctccagaacgccgagaacgactacatcaacgcgtccctggtcgacatcgaagaggcacagcgctcttacatcctgacccagggtccgctgccgaatacctgttgtcatttctggttgatggtctggcagcagaaaacgaaagctgttgtgatgctgaatcgtatcgtggagaaagaatccgtaaagtgtgcccaatactggccgaccgatgatcaagaaatgctgttcaaagaaaccggttttagcgttaagctgctgagcgaagatgtgaaaagctactatacggtgcatctgctgcaactggagaacattaacagcggcgaaacccgtaccatcagccactttcactacaccacgtggccagactttggtgttccggagtcgccggcaagcttccttaactttctgttcaaggttcgcgagtccggtagcctgaacccggatcatggtccggcagttatccactgcagcgctggcattggccgtagcggtacctttagcttggtcgacacgtgcctggtgctgatggaaaagggtgacgacattaacattaaacaagttctgctgaatatgcgcaaatatcgcatgggtctgatccagacgccggatcagctgcgtttctcttacatggcgattattgagggcgccaaatgcattaagggcgatagcagcatccagaagcgttggaaagaactgagcaaggaagatttgagccctgcgttcgatcactccccaaataagatcatgaccgagaagtataactaa

SEQ ID NO: 3 - protein, 328 aa, synthetic construct
MHHHHHHENLYFQGMPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYN